(19) United States
(12) Patent Application Publication
(10) Pub. No.: US 2023/0159449 A1
(43) Pub. Date: May 25, 2023

(54) LIPID FORMULATIONS CONTAINING NUCLEIC ACIDS AND METHODS OF TREATMENT FOR CYSTIC FIBROSIS

(71) Applicant: Arcturus Therapeutics, Inc., San Diego, CA (US)

(72) Inventors: Carlos G. PEREZ-GARCIA, San Diego, CA (US); Kiyoshi TACHIKAWA, San Diego, CA (US); Daiki MATSUDA, San Diego, CA (US); Padmanabh CHIVUKULA, San Diego, CA (US); Priya Prakash KARMALI, San Diego, CA (US); Yanjie BAO, San Diego, CA (US); Jerel Boyd Lee VEGA, San Diego, CA (US); Rajesh MUKTHAVARAM, San Diego, CA (US); Amit SAGI, San Diego, CA (US); Yihua PEI, San Diego, CA (US)

(21) Appl. No.: 18/052,505

(22) Filed: Nov. 3, 2022

Related U.S. Application Data
(60) Provisional application No. 63/275,402, filed on Nov. 3, 2021.

(51) Int. Cl. C07C 333/04; A61K 9/14; A61K 9/127; C07D 519/00; A61K 47/24

(52) U.S. Cl. CPC C07C 333/04; A61K 9/14; A61K 9/1272; C07D 519/00; A61K 47/24

(57) ABSTRACT

Lipid formulations that encapsulate messenger RNA (mRNA) are provided herein. The mRNA can be used to express CFTR protein in vitro or in vivo. The lipid formulations can be administered via inhalation to treat cystic fibrosis.

### CROSS REFERENCE TO RELATED APPLICATION

This application claims priority to U.S. Provisional Application No. 63/275,402, filed Nov. 3, 2021, which is incorporated herein by reference in its entirety.

### TECHNICAL FIELD

The present disclosure relates to lipid formulations for mRNA delivery, mRNA sequences, compositions, and methods for the treatment of cystic fibrosis. More specifically, disclosed herein are lipid formulations for the delivery of mRNA sequences for expressing a Cystic Fibrosis Transmembrane Conductance Regulator (CFTR) protein or a fragment thereof in a lung of a subject.

### REFERENCE TO SEQUENCE LISTING

The instant application contains a Sequence Listing, which has been submitted electronically in ASCII format and is hereby incorporated by reference in its entirety. Said ASCII copy, created on Nov. 3, 2022 is named “049386-547001US_ST.26_SL” and is 657,433 bytes in size.

### BACKGROUND

Cystic fibrosis (CF) is an autosomal inherited disorder resulting from mutation of the CFTR gene, which encodes a chloride ion channel believed to be involved in regulation of several other ion channels and transport systems in epithelial cells. The CFTR protein helps to maintain the balance of salt and water on many surfaces in the body, such as the surface of the lung. When the protein is not expressed properly or not working correctly, chloride becomes trapped in cells. Without the proper movement of chloride, water cannot hydrate the cellular surface. The mucus covering the cells then becomes thick and sticky, causing many of the symptoms associated with cystic fibrosis. When the CFTR gene has detrimental mutations, the corresponding loss of function of the CFTR gene results in chronic lung disease, aberrant mucus production, and dramatically reduced life expectancy.

Currently, there is no cure for CF, but there are several therapies aimed at alleviating the adverse effects of CF, and the management of CF has improved significantly over the years. Seventy years ago, infants born with CF were unlikely to live beyond their first year, but today they can live well into adulthood. The current standard of care for CF patients includes proactive treatment of airway infection and inflammation with the aim of maximizing organ function and improving quality of life for patients. However, the best possible outcome with currently available treatments is a delay in the decline of organ function.

Several gene therapies have been proposed as a means to treat CF, however each of these is associated with undesirable effects or significant challenges. For example, despite the successful cloning of the CFTR gene in 1989, there have been numerous difficulties encountered in attempting to induce expression of CFTR in the lung. Some of these previous attempts have included viral vectors comprising CFTR DNA, which induced an immune response, and CF symptoms persisted after administration of the viral vector.

One potential therapy involves the delivery of mRNA encoding a CFTR protein to the lung epithelium of a CF patient. However, mRNA-based therapies face several obstacles including achieving an adequate in vivo half-life of the mRNA, achieving an adequate translation efficiency of the mRNA such that an effective amount of enzyme is produced, minimizing adverse reactions to the mRNA (e.g., immunogenicity), and effectively delivering the mRNA to a target cell type. Another difficulty in inducing CFTR expression in the lung of a subject pertains to the lung environment. Lung-specific difficulties have been reported for mRNA delivery using certain lipoplex formulations. For example, a comparison of in vitro and in vivo performance of lipoplexes carrying mRNA or DNA revealed that even though the mRNA composition gave higher expression in cultured cells, measurable expression was detected only with the DNA composition when administered intranasally to a mouse lung (Andries et al., Mol. Pharmaceut. 9, 2136-45, 2012).

Moreover, CFTR is a large gene when compared to model or reporter genes such as firefly luciferase (FFL), which are commonly used for proof of concept studies in mRNA-based therapies. In studies on the effect of coding sequence length that compared wild-type CFTR and FFL, it was determined that the difference in length can impact stability and whether and how much protein expression any given dose of mRNA will produce. Furthermore, the production of large mRNAs for therapy can be challenging. Generally, in vitro synthesis of mRNA is preferred to cellular synthesis due to the absence of normal cellular mRNA and other cellular components that constitute undesirable contaminants. However, in vitro synthesis of mRNA with a long coding sequence, such as CFTR mRNA, is substantially more difficult to achieve than in vitro synthesis of mRNA with a relatively short coding sequence as longer sequences provide more opportunities for transcription errors and the formation of undesirable by-products.

Another challenge associated with mRNA-based therapies is associated with the effective, specific, and non-toxic delivery of the mRNA to a target cell. One method for delivering nucleic acids to target cells that has been successfully employed is the encapsulation of the nucleic acid in a lipid formulation such as a liposome or a lipid nanoparticle. While the use of lipid formulations has had some success, it has been found that several of the lipids used in these formulations show low in vivo degradability, low potency, and the potential to cause adverse reactions.

In the light of challenges highlighted above, there remains a need for improved drug product, formulations, production methods, and delivery methods of CFTR mRNA for induction of CFTR expression in the treatment of CF.

U.S. patent application Ser. No. 17/246,558 filed Apr. 30, 2021, the entire content of which is hereby incorporated by reference in its entirety, discloses mRNA sequences, compositions, and methods for the treatment of cystic fibrosis.

### SUMMARY

Additional features and advantages of the subject technology will be set forth in the description below, and in part will be apparent from the description, or may be learned by practice of the subject technology. It is to be understood that both the foregoing general description and the following detailed description are exemplary and explanatory and are intended to provide further explanation of the subject technology.

In a general aspect, disclosed herein is a composition comprising:

a. a lipid formulation comprising
i. about 20 mol % to about 30 mol % of an ionizable cationic lipid having the structure of ATX-012 (LIPID 3):

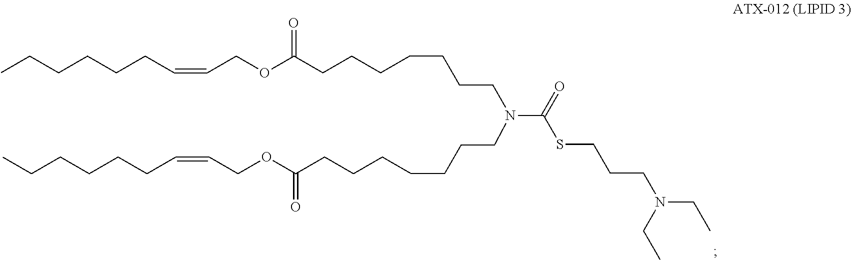 [SMILES: CCCCCC/C=C\COC(=O)CCCCCCCN(CCCCCCCC(=O)OC/C=C\CCCCCC)C(=O)SCCCN(CC)CC]

ii. about 20 mol % to about 30 mol % 1,2-Dioleoyl-3-Trimethylammonium-Propane (DOTAP);
iii. about 7 mol % to about 13 mol % of a helper lipid;
iv. about 33 mol % to about 44 mol % cholesterol; and
v. about 0.5 mol % to about 3.0 mol % of a PEG-lipid conjugate; and

b. a messenger RNA (mRNA) encoding a peptide having cystic fibrosis transmembrane conductance regulator (CFTR) activity;

wherein the lipid formulation encapsulates the mRNA.

In a further aspect, the lipid formulation of the composition can be selected from the group consisting of a lipoplex, a liposome, a lipid nanoparticle, a polymer-based carrier, an exosome, a lamellar body, a micelle and an emulsion. In yet a further aspect, the lipid formulation of the composition can be a liposome selected from the group consisting of a cationic liposome, a nanoliposome, a proteoliposome, a unilamellar liposome, a multilamellar liposome, a ceramide-containing nanoliposome and a multivesicular liposome. In yet a further aspect, the lipid formulation of the composition can be a lipid nanoparticle. In a more particular aspect, the lipid nanoparticle can have a size (diameter) of less than about 200 nm. In a further aspect, the lipid nanoparticle can have a size (diameter) of less than about 150 nm. In yet a further aspect, the lipid nanoparticle can have a size (diameter) of less than about 100 nm. In yet a further aspect still, the lipid nanoparticle can have a size (diameter) of about 55 nm to about 90 nm.

In a further aspect, the helper lipid of the composition can be a phospholipid. In yet a further aspect, the helper lipid of the composition can be selected from the group consisting of dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC) and phosphatidylcholine (PC). In a more particular aspect, the helper lipid of the composition can be distearoylphosphatidylcholine (DSPC).

In a further aspect, the lipid formulation of the composition can comprise about 8 mol % to about 12 mol % of the helper lipid. In yet a further aspect, the lipid formulation of the composition can comprise about 9 mol % to about 11 mol % of the helper lipid.

In a further aspect, the PEG-lipid conjugate of the composition can be PEG-DMG. In yet a further aspect, the PEG-DMG can be PEG2000-DMG.

In a further aspect, the lipid formulation of the composition can comprise about 0.75 mol % to about 2.5 mol % of the PEG-lipid conjugate. In yet a further aspect, the lipid formulation of the composition can comprise about 1.0 mol % to about 2.0 mol % of the PEG-lipid conjugate. In a more particular aspect, the lipid formulation of the composition can comprise about 1.25 mol % to about 1.75 mol % of the PEG-lipid conjugate.

In a further aspect, the composition can have a total lipid:mRNA weight ratio of about 5:1 to about 25:1. In yet a further aspect, the composition can have a total lipid:mRNA weight ratio of about 10:1 to about 20:1. In a further aspect still, the composition can have a total lipid:mRNA weight ratio of about 12:1 to about 18:1. In a more particular aspect, the composition can have a total lipid:mRNA weight ratio of about 14:1 to about 17:1.

In a further aspect, the lipid formulation of the composition can comprise about 22 mol % to about 28 mol % of the ionizable cationic lipid. In yet a further aspect, the lipid formulation of the composition can comprise about 23 mol % to about 27 mol % of the ionizable cationic lipid. In a further aspect still, the lipid formulation of the composition can comprise about 24 mol % to about 26 mol % of the ionizable cationic lipid.

In a further aspect, the lipid formulation of the composition can comprise about 22 mol % to about 28 mol % DOTAP. In yet a further aspect, the lipid formulation of the composition can comprise about 23 mol % to about 27 mol % DOTAP. In a more particular aspect, the lipid formulation can comprise about 24 mol % to about 26 mol % DOTAP.

In a further aspect, the lipid formulation of the composition can comprise about 35 mol % to about 41 mol % cholesterol. In yet a further aspect, the lipid formulation of the composition can comprise about 36 mol % to about 40 mol % cholesterol.

In a further aspect, the peptide of the composition having CFTR activity can have a sequence at least about 85% identical to a sequence of SEQ ID NO: 99. In yet a further aspect, the peptide having CFTR activity can have a sequence at least about 90% identical to a sequence of SEQ ID NO: 99. In yet a further aspect, the peptide having CFTR activity can have a sequence at least about 95% identical to a sequence of SEQ ID NO: 99. In a further aspect still, the peptide having CFTR activity can have a sequence at least about 98% identical to a sequence of SEQ ID NO: 99. In a more particular aspect, the peptide having CFTR activity can have a sequence at least about 99% identical to a sequence of SEQ ID NO: 99. In a more particular aspect still, the peptide having CFTR activity can have a sequence of SEQ ID NO: 99.

In a further aspect, the mRNA of the composition can have a sequence selected from the group consisting of SEQ ID NO: 49, 53, 66, 68, 69 and 72. In one further aspect, the mRNA can comprise SEQ ID NO: 49. In another further aspect, the mRNA can comprise SEQ ID NO: 53. In another further aspect, the mRNA can comprise SEQ ID NO: 66. In another further aspect, the mRNA can comprise SEQ ID NO: 68. In yet another further aspect, the mRNA can comprise SEQ ID NO: 69. In another further aspect still, the mRNA can comprise SEQ ID NO: 72.

In a further aspect, the mRNA of the composition can comprise a 3′ poly-A tail consisting of about 50 to about 120 adenosine monomers.

In a further aspect, the mRNA of the composition can comprise a 5′ cap. In yet a further aspect, the 5′ cap can be m⁷GpppAmpG having the structure of Formula (Cap V):

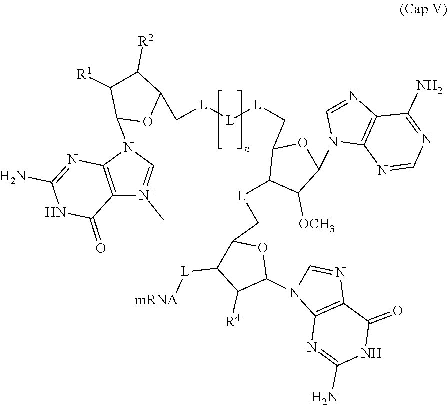

wherein R¹, R², and R⁴ are each OH, n is 1, each L is a phosphate linked by diester bonds, and mRNA is the mRNA of the composition.

In a further aspect, the mRNA of the composition can comprise one or more chemically-modified nucleotides each independently selected from the group consisting of 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine and 6-O-methylguanosine. In yet a further aspect, the one or more chemically-modified nucleotides can be N¹-methylpseudouridines.

In a further aspect, the composition can comprise a HEPES or TRIS buffer at a pH of about 7.0 to about 8.5. In yet a further aspect, the HEPES or TRIS buffer pH is about 7.4 to about 8.2. In another aspect, the HEPES or TRIS buffer can be at a concentration of about 20 mM to about 80 mM. In one particular aspect, the buffer can be HEPES at a concentration of about 35 mM to about 70 mM. In a more particular aspect, the buffer can be HEPES at a concentration of about 40 mM to about 60 mM. In yet a more particular aspect, the buffer can be HEPES at a concentration of about 45 mM to about 55 mM. In another particular aspect, the buffer can be TRIS at a concentration of about 20 mM to about 50 mM. In a more particular aspect, the buffer can be TRIS at a concentration of about 25 mM to about 40 mM. In yet a more particular aspect, the buffer can be TRIS at a concentration of about 25 mM to about 35 mM.

In a further aspect, the composition can further comprise about 10 mM to about 100 mM of NaCl. In yet a further aspect, the composition can comprise about 20 mM to about 90 mM of NaCl. In yet a further aspect, the composition can comprise about 30 mM to about 80 mM of NaCl. In an even further aspect, the composition can comprise about 35 mM to about 70 mM of NaCl. In a more particular aspect, the composition can comprise about 40 mM to about 60 mM of NaCl. In a more particular aspect still, the composition can comprise about 45 mM to about 55 mM of NaCl.

In a further aspect, the composition can further comprise one or more cryoprotectants. In a further aspect, the one or more cryoprotectants of the composition can be selected from the group consisting of sucrose, glycerol, and a combination of sucrose and glycerol. In one aspect, the cryoprotectant can be sucrose. In another aspect, the cryoprotectant can be glycerol. In yet another aspect, the cryoprotectant can be a combination of sucrose and glycerol. In a further aspect, the composition can comprise a combination of sucrose at a concentration of about 5% w/v to about 18% w/v and glycerol at a concentration of about 1% w/v to about 9% w/v. In yet a further aspect, the composition can comprise a combination of sucrose at a concentration of about 6% w/v to about 16% w/v and glycerol at a concentration of about 1.5% w/v to about 7% w/v. In yet a further aspect, the composition can comprise a combination of sucrose at a concentration of about 7% w/v to about 14% w/v and glycerol at a concentration of about 1.75% w/v to about 6% w/v. In a more particular aspect, the composition can comprise a combination of sucrose at a concentration of about 7% w/v to about 12% w/v and glycerol at a concentration of about 1% w/v to about 6% w/v. In a more particular aspect still, the composition can comprise a combination of sucrose at a concentration of about 8% w/v to about 11% w/v and glycerol at a concentration of about 3% w/v to about 6% w/v.

In a further aspect, the helper lipid of the composition can be distearoylphosphatidylcholine (DSPC); the PEG-lipid conjugate of the composition can be PEG2000-DMG; and the mRNA of the composition can comprise SEQ ID NO: 53. In yet a further aspect, the peptide of the composition having CTFR activity can have a sequence at least about 90% identical to a sequence of SEQ ID NO: 99. In a further aspect, the composition can have a total lipids:mRNA weight ratio of about 15:1. In a further aspect, the lipid formulation of the composition can be a lipid nanoparticle. In yet a further aspect, the lipid nanoparticle can have a size of less than about 100 nm. In a further aspect, lipid formulation of the composition comprises about 25 mol % ATX-012, about 25 mol % DOTAP, about 10 mol % DSPC, about 38.5 mol % cholesterol, and about 1.5 mol % PEG2000-DMG.

In another general aspect, disclosed herein is use of a composition of the present disclosure for manufacturing a medicament for ameliorating, preventing, delaying onset, or treating a disease or disorder associated with reduced activity of Cystic Fibrosis Transmembrane Conductance Regulator (CFTR) in a subject need thereof. In a further aspect, the disease can be Cystic Fibrosis having a Cystic Fibrosis mutation selected from the group consisting of Class 1A, Class 1B, Class 3, Class 4, Class 5 and Class 6. In one aspect, the Cystic Fibrosis mutation can be Class 1A. In another aspect, the Cystic Fibrosis mutation can be Class 1B. In another aspect, the Cystic Fibrosis mutation can be Class 3. In another aspect, the Cystic Fibrosis mutation can be Class 4. In another aspect, the Cystic Fibrosis mutation can be Class 5. In yet another aspect, the Cystic Fibrosis mutation is Class 6.

In another general aspect, disclosed herein is a method for ameliorating, preventing, delaying onset, or treating a disease or disorder associated with reduced activity of Cystic Fibrosis Transmembrane Conductance Regulator (CFTR) in a subject in need thereof, the method comprising administering to the subject a composition of the present disclosure. In a further aspect, the disease can be Cystic Fibrosis. In a further aspect, the administration can be intravenous, subcutaneous, pulmonary, intramuscular, intraperitoneal, dermal, oral, nasal, or inhalation. In another further aspect, the administration can be nasal or inhalation. In a more particular aspect, the administration can be inhalation. In another aspect, the administration can be once daily, weekly, biweekly, or monthly. In a further aspect, the administration can comprise administration of an effective dose of from about 0.01 to about 10 mg/kg of the mRNA in the composition. In another aspect, the administration can increase expression of CFTR in the lung epithelium.

In another general aspect, disclosed herein is a method of expressing a CFTR protein in a cell comprising contacting the cell with a composition of the present disclosure.

In another general aspect, disclosed herein is a kit for expressing a human CFTR in vivo, the kit comprising a composition of the present disclosure and a device for administering the dose. In a further aspect, the device can be an injection needle, an intravenous needle, or an inhalation device. In a more particular aspect, the device can be an inhalation device.

### BRIEF DESCRIPTION OF THE DRAWINGS

Various features of illustrative embodiments of the disclosures are described below with reference to the drawings. The illustrated embodiments are intended to illustrate, but not to limit, the disclosures.

FIG. 1 shows the correlation of hCFTR protein expression levels for various hCFTR constructs determined by In-Cell Western (ICW) and On-Cell Western (OCW) using a human CFTR antibody for codon-optimized sequences as described in Example 3.

FIG. 2 shows expression levels of UTR-optimized hCFTR mRNA sequences measured at 24 hours and 48 hours post transfection by ICW using a hCFTR specific antibody as described in Example 4.

FIG. 3 shows C-band (fully mature and glycosylated) CFTR protein levels expressed in vitro with different codon-optimized hCFTR mRNAs analyzed using Western Blot (WB) as described in Example 5.

FIG. 4 shows expression levels measured by quantifying the C-band using Western Blot as described in Example 5.

FIG. 5 shows hCFTR-specific band expression levels for cytosolic (Cyto) and membrane (Mb) fractions collected from cells transfected by hCFTR mRNA and analyzed by Western Blot (WB) using a primary antibody specific for hCFTR and for plasma membranes (sodium potassium ATPase) as described in Example 6.

FIG. 6 shows confocal immunofluorescence images of CFBE cells transfected with a codon-optimized hCFTR mRNA (SEQ ID NO: 53) and processed for immunofluorescence using an antibody specific for hCFTR protein as described in Example 7.

FIG. 7 shows the dose response for protein expression of different hCFTR mRNAs in transfected FRT cells as described in Example 8.

FIG. 8 shows transfection efficiency in FRT cells transfected with mCherry mRNA as described in Example 9. The panels on the top show the transfected (mCherry) cells, and the panels on the bottom show untransfected cells.

FIG. 9 shows ion channel conductivity measurements (Gt values) in FRT cells transfected with different mRNAs and negative controls as described in Example 10.

FIG. 10 shows ion channel conductivity measurements (Gt values) in FRT cells transfected with different mRNAs and negative controls as described in Example 10.

FIG. 11 shows ion channel conductivity measurements (Gt values) in FRT cells transfected with different mRNAs and negative controls as described in Example 10.

FIG. 12 shows ion channel conductivity measurements (Gt values) in FRT cells transfected with different mRNAs and negative controls as described in Example 10.

FIGS. 13A-13B show IFN-α immunostimulatory levels for selected lipid formulated mRNAs as described in Example 11 for (FIG. 13A) Donor 1, and (FIG. 13B) Donor 2.

FIGS. 14A-14B show IL-6 immunostimulatory levels for selected lipid formulated mRNAs as described in Example 11 for (FIG. 14A) Donor 1, and (FIG. 14B) Donor 2.

FIGS. 15A-15B show TNF-α immunostimulatory levels for selected lipid formulated mRNAs as described in Example 11 for (FIG. 15A) Donor 1, and (FIG. 15B) Donor 2.

FIG. 16 shows quantitative PCR (qPCR) measurements of mRNA levels in CF sputum for selected hCFTR mRNA-lipid formulations incubated for 24 hours as described in Example 12.

FIG. 17 shows luminescence images for lipid formulated luciferase mRNAs administered to wild-type rats intratracheally (top panel) and via nose-only nebulization (bottom panel) as described in Example 13.

FIG. 18 shows eGFP immunohistochemistry images for PBS controls as a comparison against lipid formulated eGFP mRNA treated animals as described in Example 14.

FIG. 19 shows eGFP immunohistochemistry images for lipid formulated eGFP mRNA treated animals as described in Example 14.

FIG. 20 shows TdTomato (TdT) fluorescence imaging for lung samples derived from transgenic floxed-TdTomato mice after administration of a CRE mRNA-lipid formulation as described in Example 15.

FIG. 21 shows fluorescence imaging for lung samples derived from floxed-TdTomato mice after administration of a CRE mRNA-lipid formulation and further processed with FoxJ1 and DAPI stains as described in Example 16. High magnifications of co-localization of TdT and FoxJ1 are shown in the bottom panel.

FIGS. 22A-22D show cellular profiling of the nasal epithelia by fluorescence imaging for samples derived from floxed-TdTomato mice after administration of a CRE mRNA-lipid formulation and further processed with FoxJ1 and DAPI stains as described in Example 17. (FIG. 22A) Panoramic view of the nasal septa. (FIG. 22B) High magnification images of the area indicated by the dashed rectangle in 22A. (FIG. 22C) High magnification images of the area indicated by the dashed rectangle in 22A. (FIG. 22D) Quantitative plot of cell counts for all cells expressing TdTomato (TdT+) as well as cells expressing both TdTomato and FoxJ1 (FoxJ1+/TdT+).

FIG. 23 shows fluorescence imaging for lung samples derived from floxed-TdTomato mice after administration of selected CRE mRNA-lipid formulations and further processed with FoxJ1 and DAPI stains as described in Example 18.

FIG. 24 shows the mRNA levels over time quantified by Quantigene® Assay for CFTR knockout (KO) mice treated intratracheally with different dose levels of lipid formulated-hCFTR mRNA as described in Example 19.

FIG. 25 shows hCFTR protein levels in membrane (Mb) and cytosolic (Cyt) fractions analyzed by WB using an antibody specific for hCFTR for CFTR knockout (KO) mice treated intratracheally with different dose levels of lipid formulated-hCFTR mRNA as described in Example 20.

FIG. 26 shows hCFTR mRNA levels quantified by Quantigene® Assay in samples derived from rats after 6 hours or 24 hours post-exposure for different exposure time lengths as described in Example 21.

FIG. 27 shows hCFTR mRNA levels quantified by Quantigene® Assay on nasal epithelium samples of CFTR KO mice treated with lipid formulated-hCFTR mRNA at 6 hours, 40 hours, and 60 hours post last-dose as described in Example 22.

FIG. 28 shows chloride channel current measured by Nasal Potential Difference (NPD) at 40 hours and 60 hours post last-dose in CFTR KO mice treated with a lipid formulated-hCFTR mRNA as described in Example 22.

FIG. 29 shows chloride channel current measured by Nasal Potential Difference at 40 hours and 60 hours post last-dose in CFTR KO mice treated with different hCFTR mRNA-lipid formulations as described in Example 23.

FIG. 30 shows average droplet size measurements for aerosolized lipid particles as described in Example 24.

FIG. 31 shows percentage mRNA encapsulation measured by RiboGreen assay for various lots of mRNA lipid formulation both before and after nebulization as described in Example 25.

FIG. 32 shows the percent recovery of mRNA measured by RiboGreen assay for lipid formulated mRNAs both pre- and post-nebulization as described in Example 25.

FIG. 33 shows eGFP fluorescence levels for a lipid formulated eGFP mRNA used to transfect CFBE cells pre- and post-nebulization as described in Example 26.

FIG. 34 shows eGFP fluorescence levels for a lipid formulated eGFP mRNA used to transfect CFBE cells at different doses pre- and post-nebulization using a vibrating mesh nebulizer as described in Example 27.

FIG. 35 shows eGFP protein quantification for three different dose levels of a eGFP mRNA-lipid formulation (LF-1) administered to lung tissue from a non-CF subject processed for WB and analyzed for eGFP expression at 24 hours post incubation as described in Example 28.

FIG. 36 shows eGFP protein quantification for three different dose levels of a eGFP mRNA-lipid formulation (LF-2) administered to lung tissue from a non-CF subject processed for WB and analyzed for eGFP expression at 24 hours post incubation as described in Example 28.

FIG. 37 shows eGFP protein quantification for three different dose levels of a eGFP mRNA-lipid formulation (LF-3) administered to lung tissue from a non-CF subject processed for WB and analyzed for eGFP expression at 24 hours post incubation as described in Example 28.

FIG. 38 shows eGFP protein quantification for three different dose levels of a eGFP mRNA-lipid formulation (LF-1) administered to lung tissue from a CF subject processed for WB and analyzed for eGFP expression at 24 hours post incubation as described in Example 29.

FIG. 39 shows eGFP protein quantification for three different dose levels of a eGFP mRNA-lipid formulation (LF-2) administered to lung tissue from a CF subject processed for WB and analyzed for eGFP expression at 24 hours post incubation as described in Example 29.

FIG. 40 shows eGFP protein quantification for three different dose levels of a eGFP mRNA-lipid formulation (LF-3) administered to lung tissue from a CF subject processed for WB and analyzed for eGFP expression at 24 hours post incubation as described in Example 29.

FIG. 41 shows hCFTR expression levels for selected mRNAs, reference mRNA, and a comparative mRNA transfected into CFBE cells at ascending dose levels as described in Example 30.

FIG. 42A-42D show delivery of lipid-formulated mRNA to ferret lung epithelial cells, as described in Example 31. (FIG. 42A) eGFP expression indicates clear delivery of CRE mRNA to epithelial cells in animals treated with CRE mRNA-lipid formulation (bright staining surrounding the airway). (FIG. 42B) eGFP expression indicates clear delivery of CRE mRNA to epithelial cells in animals treated with CRE mRNA-lipid formulation (bright staining surrounding the airway). (FIG. 42C) eGFP expression indicates clear delivery of CRE mRNA to epithelial cells in animals treated with CRE mRNA-lipid formulation (bright staining surrounding the airway). (FIG. 42D) Untreated controls showed only TdTomato expression due to a lack of CRE recombination.

FIGS. 43A-43D show delivery of lipid-formulated mRNA to non-human primate (NHP) lung epithelial cells, as described in Example 32. (FIG. 43A) NHPs treated with lipid formulated-TdTomato mRNA showed clear mRNA delivery to ciliated-like cells in epithelial airways, as seen by dark staining of cells lining the airway. (FIG. 43B) NHPs treated with lipid formulated-TdTomato mRNA showed clear mRNA delivery to ciliated-like cells in epithelial airways, as seen by dark staining of cells lining the airway. (FIG. 43C) NHPs treated with lipid formulated-TdTomato mRNA showed clear mRNA delivery to ciliated-like cells in epithelial airways, as seen by dark staining of cells lining the airway. (FIG. 43D) NHPs treated with PBS control showed no TdTomato expression.

FIG. 44 shows delivery of lipid-formulated mRNA to ciliated epithelial cells of ferret lungs, as described in Example 33.

FIG. 45 shows intranasal administration of LNP-hCFTR mRNA in a Class I CFTR knockout (KO) mouse model, as described in Example 34.

FIG. 46 shows the effect of administering single doses as compared to multiple doses of LNP-hCFTR mRNA, as described in Example 35.

FIG. 47 shows delivery of LNP-hCFTR to ferret bronchial epithelial (FBE) cells carrying a CFTR G551D mutation, as described in Example 36.

FIGS. 48A-48B show delivery of LNP-mRNA to human bronchial epithelial (HBE) cells, as described in Example 37. (FIG. 48A) immunocytology; (FIG. 48B) quantitation of immunocytology results.

FIGS. 49A-49C show the delivery of LNP-mRNA to in vitro and in vivo as described in Example 39. (FIG. 49A) Cell viability in CFBE cells; (FIG. 49B) Tdtomato expression in CFBE cells; (FIG. 49C) Mouse lung TdTomato immunohistochemistry images.

FIGS. 50A-50G show the delivery of LNP-mRNA to in vitro and in vivo as described in Example 41. (FIG. 50A) Cell viability in CFBE cells after transfection. (FIG. 50B) Tdtomato expression in CFBE cells after transfection. (FIG. 50C) Cell viability in CFBE cells after transfection. (FIG. 50D) Tdtomato expression in CFBE cells after transfection. (FIG. 50E) Cell viability in CFBE cells after transfection. (FIG. 50F) Tdtomato expression in CFBE cells after transfection. (FIG. 50G) Mouse lung tdTomato immunohistochemistry images.

FIGS. 51A-51F show the delivery of LNP-mRNA to in vitro and in vivo, as described in Example 42. (FIG. 51A) Cell viability in CFBE cells after transfection. (FIG. 51B) Tdtomato expression in CFBE cells after transfection. (FIG. 51C) Mouse lung tdTomato immunohistochemistry images. (FIG. 51D) Cell viability in CFBE cells after transfection. (FIG. 51E) Tdtomato expression in CFBE cells after transfection. (FIG. 51F) Mouse lung tdTomato immunohistochemistry images.

FIGS. 52A-52K show characteristics of lipid nanoparticle formulations prepared with various buffer components, as described in Example 44. (FIG. 52A) Cell viability in CFBE cells after transfection. (FIG. 52B) Tdtomato expression in CFBE cells after transfection. (FIG. 52C) Cell viability in CFBE cells after transfection. (FIG. 52D) Tdtomato expression in CFBE cells after transfection. (FIG. 52E) Particle size evaluation of different concentrations after storage under −70° C. or −20° C. long-term storage. (FIG. 52F) Particle size evaluation of different concentrations after storage under −70° C. or −20° C. long-term storage. (FIG. 52G) Particle size evaluation of formulations with different storage buffer indicated in the Table 32. (FIG. 52H) Particle size evaluation of formulations with different storage buffer indicated in the Table 32. (FIG. 52I) Particle size evaluation of formulations with different storage buffer indicated in the Table 32. (FIG. 52J) mRNA purity evaluation of the formulations indicated in the Table 32 at RT storage. (FIG. 52K) pH evaluation of the formulations indicated in the Table 32 at RT storage.

FIGS. 53A-53C show lipid nanoparticle formulation parameters after storage under a variety of conditions, as described in Example 45. (FIG. 53A) pH after storage at room temperature. (FIG. 53B) Particle size after storage at −20° C. (FIG. 53C) mRNA purity after storage at room temperature.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

In some embodiments, an mRNA encoding a Cystic Fibrosis Transmembrane Conductance Regulator (CFTR) protein is provided, wherein the mRNA comprises an open reading frame (ORF) having about 80% sequence identity with one of SEQ ID NOs: 100-105. In some embodiments, the ORF has about 85% sequence identity with one of SEQ ID NOs: 100-105. In some embodiments, the ORF has about 90% sequence identity with one of SEQ ID NOs: 100-105. In some embodiments, the ORF has about 95% sequence identity with one of SEQ ID NOs: 100-105. In some embodiments, the ORF has about 96% sequence identity with one of SEQ ID NOs: 100-105. In some embodiments, the ORF has about 97% sequence identity with one of SEQ ID NOs: 100-105. In some embodiments, the ORF has about 98% sequence identity with one of SEQ ID NOs: 100-105. In some embodiments, the ORF has about 99% sequence identity with one of SEQ ID NOs: 100-105. In some embodiments, the ORF has a sequence selected from the group consisting of SEQ ID NOs: 100-105. In some embodiments, the ORF has the sequence of SEQ ID NO: 100. In some embodiments, the ORF has the sequence of SEQ ID NO: 101. In some embodiments, the ORF has the sequence of SEQ ID NO: 102. In some embodiments, the ORF has the sequence of SEQ ID NO: 103. In some embodiments, the ORF has the sequence of SEQ ID NO: 104. In some embodiments, the ORF has the sequence of SEQ ID NO: 105.

In some embodiments, the mRNA further comprises a 5′ untranslated region (5′ UTR). In some embodiments, the 5′ UTR comprises a sequence selected from SEQ ID NOs: 106-125. In some embodiments, the 5′ UTR comprises SEQ ID NO: 106.

In some embodiments, the mRNA further comprises a 3′ untranslated region (3′ UTR). In some embodiments, the 3′ UTR comprises a sequence selected from the group consisting of SEQ ID NOs: 126-145. In some embodiments, the 3′ UTR comprises SEQ ID NO: 126.

In some embodiments, the mRNA further comprises a 3′ poly-adenosine (poly-A) tail. In some embodiments, the 3′ poly-A tail consists of about 50 to about 120 adenosine monomers.

In some embodiments, the mRNA further comprises a 5′ cap. In some embodiments, the 5′ cap is m⁷GpppGm having the structure of Formula Cap IV disclosed herein wherein R¹ and R² are each OH, R³ is OCH₃, each L is a phosphate linked by diester bonds, mRNA is a mRNA of the present disclosure linked at its 5′ end, and n is 1. In some embodiments, the 5′ cap is m⁷GpppAmpG having the structure of Formula Cap V disclosed herein wherein R¹, R², and R⁴ are each OH, n is 1, each L is a phosphate linked by diester bonds, and mRNA is a mRNA of the present disclosure linked at its 5′ end.

In some embodiments, the mRNA comprises one or more chemically-modified nucleotides. In some embodiments, the one or more chemically-modified nucleotides are each independently selected from 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine, and 6-O-methylguanosine. In some embodiments, the one or more chemically-modified nucleotides are N¹-methylpseudouridines. In some embodiments, the one or more chemically-modified nucleotides are 5-methoxyuridines. In some embodiments, the one or more chemically-modified nucleotides are a combination of 5-methylcytidines and N¹-methylpseudouridines. In some embodiments, the one or more chemically-modified nucleotides are a combination of 5-methoxyuridines and N¹-methylpseudouridines. In some embodiments, the one or more chemically-modified nucleotides are a combination of 5-methoxyuridines, 5-methylcytidines and N¹-methylpseudouridines. In some embodiments, the one or more chemically-modified nucleotides comprise 1-99% of the nucleotides. In some embodiments, the one or more chemically-modified nucleotides comprise 50-99% of the nucleotides.

In some embodiments, the ORF is translatable in a mammalian cell to express the human CFTR protein having CFTR activity. In some embodiments, the ORF is translatable in a subject in vivo to express the human CFTR protein having CFTR activity.

In some embodiments, the mRNA comprises a sequence selected from SEQ ID NOs: 49, 53, 66, 68, 69, and 72. In some embodiments, the mRNA comprises SEQ ID NO: 49. In some embodiments, the mRNA comprises SEQ ID NO: 53. In some embodiments, the mRNA comprises SEQ ID NO: 66. In some embodiments, the mRNA comprises SEQ ID NO: 68. In some embodiments, the mRNA comprises SEQ ID NO: 69. In some embodiments, the mRNA comprises SEQ ID NO: 72.

In some embodiments, a pharmaceutical composition comprising an mRNA of the present disclosure and a lipid of Formula I or a pharmaceutically acceptable salt or solvate thereof is provided, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O— or —OC(O)—; X⁶ is —C(O)O— or —OC(O)—; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, a pharmaceutical composition comprising an mRNA of the present disclosure and a lipid selected from an ionizable cationic lipid specifically disclosed herein or a pharmaceutically acceptable salt thereof is provided.

In some embodiments, a pharmaceutical composition comprising an mRNA of the present disclosure and an ionizable cationic lipid having the structure of ATX-012:

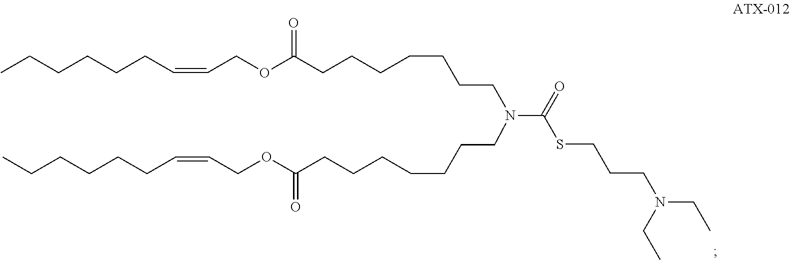 [SMILES: CCCCCC/C=C\COC(=O)CCCCCCCN(CCCCCCCC(=O)OC/C=C\CCCCCC)C(=O)SCCCN(CC)CC]

or a pharmaceutically acceptable salt or solvate thereof is provided.

In some embodiments, the pharmaceutical composition further comprises a pharmaceutically acceptable carrier. In some embodiments, the carrier comprises a transfection reagent, a nanoparticle, or a liposome.

In some embodiments, the pharmaceutical composition comprises a lipid formulation. In some embodiments, the lipid formulation is selected from the group consisting of a lipoplex, a liposome, a lipid nanoparticle, a polymer-based carrier, an exosome, a lamellar body, a micelle, and an emulsion. In some embodiments, the lipid formulation is a liposome. In some embodiments, the liposome is selected from the group consisting of a cationic liposome, a nanoliposome, a proteoliposome, a unilamellar liposome, a multilamellar liposome, a ceramide-containing nanoliposome, and a multivesicular liposome. In some embodiments, the lipid formulation encapsulates the mRNA. In some embodiments, the lipid formulation encapsulates at least about 50% of the mRNA.

In some embodiments, the pharmaceutical composition comprises lipid nanoparticles. In some embodiments, the lipid nanoparticles encapsulate the mRNA. In some embodiments, the lipid nanoparticles encapsulate at least about 50% of the mRNA. In some embodiments, the lipid nanoparticles comprise a cationic lipid, a helper lipid, a cholesterol, and a PEG-lipid conjugate.

In some embodiments, the lipid nanoparticles have a size less than about 200 nm. In some embodiments, the lipid nanoparticles have a size less than about 150 nm. In some embodiments, the lipid nanoparticles have a size less than about 100 nm. In some embodiments, the lipid nanoparticles have a size less than about 90 nm. In some embodiments, the lipid nanoparticles have a size less at least about 50 nM. In some embodiments, the lipid nanoparticles have a size within a range of about 50 to about 90 nm. In some embodiments, the lipid nanoparticles have a size within a range of about 55 to about 90 nm. In some embodiments, the lipid nanoparticles have an average particles size of between about 50 and about 85 nm. In some embodiments, the lipid nanoparticles have a size within a range of about 55 to about 85 nm.

In some embodiments, the pharmaceutical composition comprises a lipid formulation, wherein the lipid formulation comprises a cationic lipid, a helper lipid, a cholesterol, and a polyethylene glycol (PEG)-lipid conjugate.

In some embodiments, the lipid formulation comprises an ionizable cationic lipid. In some embodiments, lipid formulation comprises between about 20 mol % and about 30 mol % of the ionizable cationic lipid. In some embodiments, the lipid formulation comprises between about 22 mol % and about 28 mol % of the ionizable cationic lipid. In some embodiments, the lipid formulation comprises between about 23 mol % and about 27 mol % of the ionizable cationic lipid. In some embodiments, the lipid formulation comprises between about 24 mol % and about 26 mol % of the ionizable cationic lipid. In some embodiments, the lipid formulation comprises about 25 mol % of the ionizable cationic lipid. In some embodiments, the ionizable cationic lipid is ATX-012.

In some embodiments, the helper lipid is a phospholipid. In some embodiments, the helper lipid is selected from the group consisting of dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), 1,2-dioleoyl-3-trimethylammonium-propane (DOTAP), (N-[1-(2,3-dioleoyloxy)propyl]-N,N,N-trimethyl-ammonium methyl sulfate) (DOTMA), and phosphatidylcholine (PC), or combination of any of the foregoing. In some embodiments, the helper lipid is selected from the group consisting of DOPE, DMPC, DSPC, DMPG, DPPC and PC. In some embodiments, the helper lipid is distearoylphosphatidylcholine (DSPC). In some embodiments, the helper lipid is a combination of DOTAP and DSPC.

In some embodiments, the pharmaceutical composition comprises a lipid formulation, wherein the lipid formulation comprises between about 20 mol % and about 30 mol % DOTAP. Some embodiments, the lipid formulation comprises between about 22 mol % and about 28 mol % DOTAP. In some embodiments, the lipid formulation comprises between about 23 mol % and about 27 mol % DOTAP. In some embodiments, the lipid formulation comprises between about 24 mol % and about 26 mol % DOTAP. In some embodiments, the lipid formulation comprises about 25 mol % DOTAP.

In some further embodiments, the lipid formulation containing DOTAP further comprises between about 7 mol % and about 13 mol % of a second helper lipid. In some embodiments, the lipid formulation containing DOTAP further comprises between about 8 mol % and about 12 mol % of the second helper lipid. In some embodiments, the lipid formulation containing DOTAP further comprises between about 9 mol % and about 11 mol % of the second helper lipid. In some embodiments, the lipid formulation containing DOTAP further comprises about 10 mol % of the second helper lipid. In some embodiments, the second helper lipid is DSPC. Thus, in some embodiments, the lipid formulation comprises between about 20 mol % and about 30 mol % DOTAP, and between about 7 mol % and 13 mol % DSPC. In some embodiments, the lipid formulation encapsulates the mRNA. In some embodiments, the lipid formulation is a lipid nanoparticle formulation.

In some embodiments, the PEG-lipid conjugate is PEG-dimyristoyl glycerol (PEG-DMG). In some embodiments, the PEG-DMG is PEG2000-DMG.

In some embodiments, the pharmaceutical composition comprises a lipid formulation, wherein the lipid formulation comprises a PEG-lipid conjugate. In some embodiments, the lipid formulation comprises between about 0.5 mol % and about 3.0 mol % of a PEG-lipid conjugate. In some embodiments, the lipid formulation comprises between about 0.75 mol % and about 2.5 mol % of the PEG-lipid conjugate. In some embodiments, the lipid formulation comprises between about 1.0 mol % and about 2.0 mol % of the PEG-lipid conjugate. In some embodiments, the lipid formulation comprises between about 1.25 mol % and about 1.75 mol % of the PEG-lipid conjugate. In some embodiments, the lipid formulation comprises about 1.5 mol % of the PEG-lipid conjugate. In some embodiments, the PEG-lipid conjugate is PEG-DMG. In some embodiments, the PEG-DMG is PEG2000-DMG. In some embodiments, the lipid formulation encapsulates the mRNA. In some embodiments, the lipid formulation is a lipid nanoparticle formulation.

In some embodiments, the pharmaceutical composition comprises a lipid formulation, wherein the lipid formulation comprises cholesterol. In some embodiments, the lipid formulation comprises between bout 33 mol % and about 44 mol % cholesterol. In some embodiments, the lipid formulation comprises between about 35 mol % and about 41 mol % cholesterol. In some embodiments, the lipid formulation comprises between about 36 mol % and about 40 mol % cholesterol. In some embodiments, the lipid formulation comprises about 36 mol %, about 37 mol %, about 38 mol %, about 39 mol % or about 40 mol % cholesterol. In some embodiments, the lipid formulation comprises about 37 mol %, about 37.5 mol %, about 38 mol %, about 38.5 mol % or about 39 mol % cholesterol. In some embodiments, the lipid formulation comprises about 38.5 mol % cholesterol. In some embodiments, the lipid formulation encapsulates the mRNA. In some embodiments, the lipid formulation is a lipid nanoparticle formulation.

In some embodiments, the lipid nanoparticles comprise between about 20 mol % and 40 mol % of the cationic lipid; between about 25 mol % and 35 mol % of helper lipid; between about 25 mol % and 42 mol % cholesterol; and between about 0.5 mol % and 3 mol % PEG2000-DMG.

In some embodiments, the lipid nanoparticles comprise between about 20 mol % and 30 mol % of the cationic lipid; between about 30 mol % and 40 mol % of helper lipid; between about 34 mol % and 42 mol % cholesterol; and between about 1 mol % and 2 mol % PEG2000-DMG.

In some embodiments, the lipid nanoparticles comprise between about 22 mol % and 28 mol % of the cationic lipid; between about 31 mol % and 39 mol % of helper lipid; between about 35 mol % and 40 mol % cholesterol; and between about 1.25 mol % and 1.75 mol % PEG2000-DMG.

In some embodiments, the lipid formulation comprises between about 20 mol % and about 30 mol % of an ionizable cationic lipid; between about 20 mol % and about 30 mol % DOTAP; between about 7 mol % and about 13 mol % of a second helper lipid; between about 33 mol % and about 44 mol % cholesterol; and between about 0.5 mol % and about 3.0 mol % of a PEG-lipid conjugate. In some further embodiments, the ionizable cationic lipid is ATX-012, or a pharmaceutically acceptable salt thereof. In yet some further embodiments, the second helper lipid is DSPC. In yet some further embodiments still, the PEG-lipid conjugate is PEG-DMG. In some further embodiments still, the PEG-DMG is PEG2000-DMG. Thus, in some further embodiments, the lipid formulation, which can comprise lipid nanoparticles, comprises between about 20 mol % and about 30 mol % of ATX-012; between about 20 mol % and about 30 mol % DOTAP; between about 7 mol % and about 13 mol % of DSPC; between about 33 mol % and about 44 mol % cholesterol; and between about 0.5 mol % and about 3.0 mol % of PEG-DMG. In some embodiments, the lipid formulation is capable of encapsulating mRNA. In some embodiments, the lipid formulation is a lipid nanoparticle formulation.

In some embodiments, the pharmaceutical composition comprises a lipid formulation and an mRNA. In some embodiments, the mRNA encodes a peptide having CFTR activity. In some embodiments, the lipid formulation encapsulates the mRNA encoding the peptide having CTFR activity. In some embodiments, the lipid formulation is a lipid nanoparticle formulation. In some embodiments, the mRNA encodes an amino acid sequence that is at least 85% identical, at least 90% identical, at least 95% identical, at least 96% identical, at least 97% identical, at least 98% identical, at least 99% identical, or is 100% identical to SEQ ID NO: 99. In some embodiments, the mRNA comprises a sequence selected from the group consisting of SEQ ID NOs: 49, 53, 66, 68, 69 and 72. In some embodiments, the mRNA comprises SEQ ID NO: 49. In some embodiments, the mRNA comprises SEQ ID NO: 53. In some embodiments, the mRNA comprises SEQ ID NO: 66. In some embodiments, the mRNA comprises SEQ ID NO: 68. In some embodiments, the mRNA comprises SEQ ID NO: 69. In some embodiments, the mRNA comprises SEQ ID NO: 72.

In some embodiments, the pharmaceutical composition comprises a lipid formulation and an mRNA, wherein the mRNA comprises a 3′ poly-A tail. In some embodiments, the 3′ poly-A tail consists of about 50 to about 120 adenosine monomers.

In some embodiments, the pharmaceutical composition comprises a lipid formulation and an mRNA, wherein the mRNA comprises a 5′ cap. In some embodiments, the 5′ cap is m⁷GpppAmpG. In some embodiments, the m⁷GpppAmpG has the structure of Formula (CAP V):

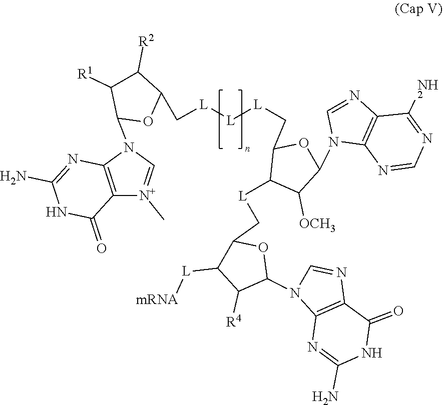

wherein R¹, R², and R⁴ are each OH, n is 1, each L is a phosphate linked by diester bonds, and mRNA is the mRNA of the composition.

In some embodiments, the mRNA of the pharmaceutical composition comprises one or more chemically-modified nucleotides each independently selected from the group consisting of 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine and 6-O-methylguanosine. In some embodiments, the one or more chemically-modified nucleotides are N¹-methylpseudouridines.

In some embodiments, the pharmaceutical composition has a total lipid:mRNA weight ratio of between about 5:1 and about 40:1. In some embodiments, the pharmaceutical composition has a total lipid:mRNA weight ratio of between about 8:1 and 40:1. In some embodiments, the pharmaceutical composition has a total lipid:mRNA weight ratio of between about 10:1 and 30:1. In some embodiments, the pharmaceutical composition has a total lipid:mRNA weight ratio of between about 15:1 and 30:1. In some embodiments, the pharmaceutical composition has a total lipid:mRNA weight ratio of between about 10:1 and 25:1. In some embodiments, the pharmaceutical composition has a total lipid:mRNA weight ratio of between about 5:1 and about 25:1. In some embodiments, the pharmaceutical composition has a total lipid:mRNA weight ratio of between about 10:1 and about 20:1. In some embodiments, the pharmaceutical composition has a total lipid:mRNA weight ratio of between about 12:1 and about 18:1. In some embodiments, the pharmaceutical composition has a total lipid:mRNA weight ratio of between about 14:1 and about 17:1. In some embodiments, the pharmaceutical composition has a total lipid:mRNA weight ratio of between about 15:1 and about 16:1.

In some embodiments, the pharmaceutical composition comprises between about 20 w/w % and 60 w/w % of the cationic lipid. In some embodiments, the pharmaceutical composition comprises between about 20 w/w % and 50 w/w % of the cationic lipid. In some embodiments, the pharmaceutical composition comprises between about 20 w/w % and 40 w/w % of the cationic lipid. In some embodiments, the pharmaceutical composition comprises between about 20 w/w % and 30 w/w % of the cationic lipid. In some embodiments, the pharmaceutical composition comprises about 25 w/w % of the cationic lipid.

In some embodiments, a pharmaceutical composition comprising a lipid formulation and an mRNA can further comprise a buffer. In some embodiments, the buffer has a pH of about 7.0 to about 8.5. In some embodiments, the buffer is a HEPES or TRIS buffer. In some embodiments, the HEPES or TRIS buffer pH is about 7.0 to about 8.5. In some embodiments, the HEPES or TRIS buffer pH is about 7.4 to about 8.2. In some embodiments, the HEPES or TRIS buffer is at a concentration of about 20 mM to about 80 mM. In some embodiments, the buffer is HEPES buffer. In some embodiments, the buffer is HEPES buffer at a concentration of about 35 mM to about 70 mM. In some embodiments, the buffer is HEPES buffer at a concentration of about 40 mM to about 60 mM. In some embodiments, the buffer is HEPES buffer at a concentration of about 45 mM to about 55 mM. In some embodiments, the buffer is TRIS buffer. In some embodiments, the buffer is TRIS buffer at a concentration of about 20 mM to about 50 mM. In some embodiments, the buffer is TRIS buffer at a concentration of about 25 mM to about 40 mM. In some embodiments, the buffer is TRIS buffer at a concentration of about 25 mM to about 35 mM.

In some embodiments, a pharmaceutical composition comprising a lipid formulation and an mRNA further comprises sodium chloride (NaCl). In some embodiments, the pharmaceutical composition comprises about 10 mM to about 100 mM of NaCl. In some embodiments, the pharmaceutical composition comprises about 20 mM to about 90 mM of NaCl. In some embodiments, the pharmaceutical composition comprises about 30 mM to about 80 mM of NaCl. In some embodiments, the pharmaceutical composition comprises about 35 mM to about 70 mM of NaCl. In some embodiments, the pharmaceutical composition comprises comprise about 40 mM to about 60 mM of NaCl. In some embodiments, the pharmaceutical composition comprises about 45 mM to about 55 mM of NaCl.

In some embodiments, a pharmaceutical composition comprising a lipid formulation and an mRNA further comprises one or more cryoprotectants. In some embodiments, the one or more cryoprotectants is selected from the group consisting of sucrose, glycerol, and a combination of sucrose and glycerol. In some embodiments, the cryoprotectant is sucrose. In some embodiments, the cryoprotectant is glycerol. In some embodiments, the cryoprotectant is a combination of sucrose and glycerol. In some embodiments, the pharmaceutical composition comprises a combination of sucrose at a concentration of about 5% w/v to about 18% w/v and glycerol at a concentration of about 1% w/v to about 9% w/v. In some embodiments, the pharmaceutical composition comprises a combination of sucrose at a concentration of about 6% w/v to about 16% w/v and glycerol at a concentration of about 1.5% w/v to about 7% w/v. In some embodiments, the pharmaceutical composition comprises a combination of sucrose at a concentration of about 7% w/v to about 14% w/v and glycerol at a concentration of about 1.75% w/v to about 6% w/v. In some embodiments aspect, the pharmaceutical composition comprises a combination of sucrose at a concentration of about 7% w/v to about 12% w/v and glycerol at a concentration of about 1% w/v to about 6% w/v. In some embodiments, the composition comprises a combination of sucrose at a concentration of about 8% w/v to about 11% w/v and glycerol at a concentration of about 3% w/v to about 6% w/v.

In some embodiments, the pharmaceutical composition is provided for use in medical therapy. In some embodiments, the pharmaceutical composition is provided for use in the treatment of the human or animal body.

In some embodiments, use of the pharmaceutical composition for manufacturing a medicament for ameliorating, preventing, delaying onset, or treating a disease or disorder associated with reduced activity of Cystic Fibrosis Transmembrane Conductance Regulator (CFTR) in a subject need thereof is provided. In some embodiments, the disease is Cystic Fibrosis having a Cystic Fibrosis mutation selected from Class 1A, Class 1, Class 3, Class 4, Class 5 and Class 6. In some embodiments, the Cystic Fibrosis mutation is Class 1A. In some embodiments, the Cystic Fibrosis mutation is Class 1B. In some embodiments, the Cystic Fibrosis mutation is Class 3. In some embodiments, the Cystic Fibrosis mutation is Class 4. In some embodiments, the Cystic Fibrosis mutation is Class 5. In some embodiments, the Cystic Fibrosis mutation is Class 6.

In some embodiments, a method for ameliorating, preventing, delaying onset, or treating a disease or disorder associated with reduced activity of Cystic Fibrosis Transmembrane Conductance Regulator (CFTR) in a subject in need thereof is provided comprising administering to the subject one or more mRNA sequences or a pharmaceutical composition described herein. In some embodiments, the disease is Cystic Fibrosis. In some embodiments, the administration is intravenous, subcutaneous, pulmonary, intramuscular, intraperitoneal, dermal, oral, nasal, or inhalation. In some embodiments, the administration is nasal or inhalation. In some embodiments, the administration is inhalation. In some embodiments, the administration is once daily, weekly, biweekly, or monthly. In some embodiments, the administration comprises an effective dose of from 0.01 to 10 mg/kg. In some embodiments, the administration increases expression of CFTR in the lung epithelium.

In some embodiments, a method of expressing a CFTR protein in a cell is provided comprising contacting the cell with one or more mRNA sequences or a pharmaceutical composition described herein.

In some embodiments, a kit for expressing a human CFTR in vivo is provided, the kit comprising a 0.1 to 500 mg dose of an mRNA or a pharmaceutical composition described herein; and a device for administering the dose. In some embodiments, the device is an injection needle, an intravenous needle, or an inhalation device. In some embodiments, the device is an inhalation device.

### Human CFTR

In some embodiments, a mRNA sequence is provided comprising an mRNA coding sequence encoding the human CFTR protein. The sequence of the naturally occurring human CFTR protein is provided in SEQ ID NO: 93.

In some embodiments, the mRNA encodes a protein substantially identical to human CFTR protein. In some embodiments, the mRNA encodes an amino acid sequence that is at least 80%, 85%, 90%, 91%, 92%, 93%, 94%, 95%, 96%, 97%, 98%, 99% or more identical to SEQ ID NO: 93. In embodiments, the mRNA encodes an amino acid sequence that is at least 80% or more identical to SEQ ID NO: 93. In embodiments, the mRNA encodes an amino acid sequence that is at least 85% or more identical to SEQ ID NO: 93. In embodiments, the mRNA encodes an amino acid sequence that is at least 90% or more identical to SEQ ID NO: 93. In embodiments, the mRNA encodes an amino acid sequence that is at least 91% or more identical to SEQ ID NO: 93. In embodiments, the mRNA encodes an amino acid sequence that is at least 92% or more identical to SEQ ID NO: 93. In embodiments, the mRNA encodes an amino acid sequence that is at least 93% or more identical to SEQ ID NO: 93. In embodiments, the mRNA encodes an amino acid sequence that is at least 94% or more identical to SEQ ID NO: 93. In embodiments, the mRNA encodes an amino acid sequence that is at least 95% or more identical to SEQ ID NO: 93. In embodiments, the mRNA encodes an amino acid sequence that is at least 96% or more identical to SEQ ID NO: 93. In embodiments, the mRNA encodes an amino acid sequence that is at least 97% or more identical to SEQ ID NO: 93. In embodiments, the mRNA encodes an amino acid sequence that is at least 98% or more identical to SEQ ID NO: 93. In embodiments, the mRNA encodes an amino acid sequence that is at least 99% or more identical to SEQ ID NO: 93. In some embodiments, the mRNA encodes a protein having hCFTR activity having the sequence of SEQ ID NO: 93. In some embodiments, an mRNA suitable for the present disclosure encodes a fragment or a portion of human CFTR protein.

In some embodiments, the disclosure provides an mRNA sequence that encodes a homolog or variant of human CFTR. As used herein, a homolog or a variant of human CFTR protein may be a modified human CFTR protein containing one or more amino acid substitutions, deletions, and/or insertions as compared to a wild-type or naturally-occurring human CFTR protein while retaining substantial CFTR protein activity. In some embodiments, the mRNA encodes a protein selected from SEQ ID NOs: 95, 96, 97, and 99, or a fragment thereof. In some embodiments, the mRNA encodes an amino acid sequence that is at least 80%, 85%, 90%, 91%, 92%, 93%, 94%, 95%, 96%, 97%, 98%, 99% or more identical to SEQ ID NOs: 95, 96, 97, and 99. In some embodiments, the mRNA encodes an amino acid sequence that is at least 80%, 85%, 90%, 91%, 92%, 93%, 94%, 95%, 96%, 97%, 98%, 99% or more identical to SEQ ID NO: 95. In some embodiments, the mRNA encodes an amino acid sequence that is at least 80%, 85%, 90%, 91%, 92%, 93%, 94%, 95%, 96%, 97%, 98%, 99% or more identical to SEQ ID NO: 96. In some embodiments, the mRNA encodes an amino acid sequence that is at least 80%, 85%, 90%, 91%, 92%, 93%, 94%, 95%, 96%, 97%, 98%, 99% or more identical to SEQ ID NO: 97. In some embodiments, the mRNA encodes an amino acid sequence that is at least 80%, 85%, 90%, 91%, 92%, 93%, 94%, 95%, 96%, 97%, 98%, 99% or more identical to SEQ ID NO: 99. In some embodiments, the mRNA encodes an amino acid sequence that is at least 80% identical to SEQ ID NO: 99. In some embodiments, the mRNA encodes an amino acid sequence that is at least 85% identical to SEQ ID NO: 99. In some embodiments, the mRNA encodes an amino acid sequence that is at least 90% identical to SEQ ID NO: 99. In some embodiments, the mRNA encodes an amino acid sequence that is at least 95% identical to SEQ ID NO: 99. In some embodiments, the mRNA encodes an amino acid sequence that is at least 98% identical to SEQ ID NO: 99. In some embodiments, the mRNA encodes an amino acid sequence that is at least 99% identical to SEQ ID NO: 99. In some embodiments, the mRNA encodes a protein having hCFTR activity having the sequence of SEQ ID NO: 99.

In some embodiments, an mRNA suitable for the present disclosure encodes a fragment or a portion of human CFTR protein, wherein the fragment or portion of the protein still maintains CFTR activity similar to or improved upon that of the wild-type protein.

In some embodiments, an mRNA suitable for the present disclosure comprises a sequence that is at least 80%, 85%, 90%, 91%, 92%, 93%, 94%, 95%, 96%, 97%, 98%, 99% or more identical to SEQ ID NOs: 49, 53, 66, 68, 69, or 72. In some embodiments, an mRNA provided herein comprises a sequence selected from SEQ ID NOs: 49, 53, 66, 68, 69, and 72. In some embodiments, an mRNA provided herein comprises SEQ ID NO: 49. In some embodiments, an mRNA provided herein comprises SEQ ID NO: 53. In some embodiments, an mRNA provided herein comprises SEQ ID NO: 66. In some embodiments, an mRNA provided herein comprises SEQ ID NO: 68. In some embodiments, an mRNA provided herein comprises SEQ ID NO: 69. In some embodiments, an mRNA provided herein comprises SEQ ID NO: 72.

In some embodiments, a mRNA of the present disclosure comprises a coding sequence that is at least 80%, 85%, 90%, 91%, 92%, 93%, 94%, 95%, 96%, 97%, 98%, 99% or more identical to SEQ ID NOs: 100, 101, 102, 103, 104, or 105. In some embodiments, an mRNA comprises a coding sequence that is at least 80%, 85%, 90%, 91%, 92%, 93%, 94%, 95%, 96%, 97%, 98%, 99% or more identical to SEQ ID NOs: 100, 101, 102, 103, 104, or 105, and further comprises one or more components selected from a 5′ cap, a 5′ UTR, a translation initiation sequence, a 3′ UTR, and a tail region. In some embodiments, an mRNA provided herein comprises a coding sequence selected from SEQ ID NOs: 100, 101, 102, 103, 104, and 105. In some embodiments, an mRNA provided herein comprises a coding sequence selected from SEQ ID NOs: 100, 101, 102, 103, 104, and 105, and further comprises one or more components selected from a 5′ cap, a 5′ UTR, a translation initiation sequence, a 3′ UTR, and a tail region.

In some embodiments, an mRNA of the disclosure provides a fusion protein comprising a full length, fragment or portion of a CFTR protein fused to another sequence (e.g., an N or C terminal fusion). In some embodiments, the N or C terminal sequence is a signal sequence or a cellular targeting sequence.

Translatable mRNA Sequences and Constructs

The compositions and methods of the present disclosure include a mRNA that encodes an active and functional CFTR protein. The mRNA can include several features that enhance its in vivo half-life and translation efficiency. In addition, the present disclosure provides for DNA scaffolds for producing an mRNA encoding an active and functional CFTR protein via transcription. The DNA scaffold can be any suitable form of DNA including a plasmid DNA. The polynucleotides contemplated by the present disclosure are further described in detail below.

An mRNA of this disclosure comprising a coding sequence encoding a functional CFTR moiety can be delivered to a patient in need (e.g., CF patient), and can elevate active CFTR levels of the patient. The mRNA sequence can be used for preventing, treating, ameliorating or reversing any symptoms of Cystic Fibrosis in the patient. As will be appreciated by the skilled artisan equipped with the present disclosure, the mRNA sequences and constructs of the present disclosure may be used to ameliorate, prevent, or treat any disease or disorder associated with reduced activity (e.g., resulting from reduced concentration, presence, and/or function) of Cystic Fibrosis Transmembrane Conductance Regulator (CFTR) and/or a disease associated with reduced presence or function of CFTR in a subject.

The mRNA sequences and constructs of this disclosure can have long half-life, particularly in the cytoplasm. They can be used for ameliorating, preventing, or treating a disease or disorder associated with reduced activity (e.g., resulting from reduced concentration, presence, and/or function) of Cystic Fibrosis Transmembrane Conductance Regulator (CFTR) in a subject.

The properties of the mRNA sequences and constructs of this disclosure arise according to their molecular structure, and the structure of the molecule in its entirety, as a whole, can provide significant benefits based on those properties. Embodiments of this disclosure can provide mRNA sequences and constructs having one or more properties that advantageously provide enhanced protein concentration or increased protein activity. The sequences and constructs can further be used in pharmaceutical compositions of this disclosure for ameliorating, preventing, or treating any disease or disorder associated with reduced activity (e.g., resulting from reduced concentration, presence, and/or function) of Cystic Fibrosis Transmembrane Conductance Regulator (CFTR) in a subject.

This disclosure herein provides a range of mRNA sequences that show a surprising degree of translatability to provide active polypeptide or protein, in vitro, ex vivo, and in vivo.

The mRNA sequences, constructs, and compositions can have increased translational activity or cytoplasmic half-life. In these embodiments, the mRNA sequences, constructs, and compositions can provide increased functional half-life in the cytoplasm of mammalian cells, as compared to a native mRNA (i.e., an mRNA transcribed in vivo from the cell's own genome).

In additional embodiments, an mRNA sequence can contain one or more UNA monomers in a 3′ untranslated region of monomers.

In further embodiments, an mRNA sequence can contain one or more UNA monomers in a tail region of monomers.

In further embodiments, an mRNA sequence can contain one or more UNA monomers in a poly-A tail.

In some embodiments, an mRNA sequence can contain one or more LNA monomers in a 3′ untranslated region of monomers or in a tail region of monomers, e.g., in a poly-A tail.

In another aspect, an mRNA sequence of this disclosure can exhibit at least 2-fold, 3-fold, 5-fold, or 10-fold increased translation efficiency in vivo as compared to a native mRNA that encodes the same translation product.

In a further aspect, an mRNA sequence can produce at least a 2-fold, 3-fold, 5-fold, or 10-fold increased polypeptide or protein level in vivo as compared to a native mRNA that encodes the same polypeptide or protein.

In certain embodiments, an mRNA sequence can provide increased levels of a polypeptide or protein in vivo as compared to a native mRNA that encodes the same polypeptide or protein. For example, the level of a polypeptide or protein can be increased by 10%, or 20%, or 30%, or 40%, or 50%, or more.

In additional embodiments, this disclosure provides methods for treating a disease or condition in a subject by administering to the subject a composition containing an mRNA sequence of the disclosure.

An mRNA sequence of this disclosure may be used for ameliorating, preventing or treating a disease or disorder, e.g., a disease or disorder associated with reduced activity (e.g., resulting from reduced concentration, presence, and/or function) of Cystic Fibrosis Transmembrane Conductance Regulator (CFTR) in a subject. In these embodiments, a composition comprising an mRNA sequence of this disclosure can be administered to regulate, modulate, or increase the concentration or effectiveness of CFTR in a subject. In one aspect, the protein can be an unmodified, natural protein for which the patient has an abnormal quantity (e.g., a patient with a mutated version of CFTR which partially or totally abolishes CFTR activity). In one aspect, the protein can be an unmodified, natural CFTR protein which can be used to treat a patient harboring a mutated version of CFTR. In embodiments, an mRNA sequence of this disclosure may be used for ameliorating, preventing or treating Cystic Fibrosis.

In some embodiments, an mRNA sequence may be delivered to cells or subjects and translated to increase CFTR levels in the cell or subject.

In an embodiment, a subject of the present disclosure is a subject with reduced activity (e.g., resulting from reduced concentration, presence, and/or function) of Cystic Fibrosis Transmembrane Conductance Regulator (CFTR). In a further embodiment, the subject is a human.

In some embodiments, administering a composition comprising an mRNA sequence of the disclosure can result in increased CFTR protein levels in a treated subject. In some embodiments, administering a composition comprising an mRNA sequence of the disclosure results in about a 5%, 10%, 20%, 30%, 40%, 50%, 60%, 70%, 80%, 90%, or 95% increase in CFTR protein levels relative to a baseline CFTR protein level in the subject prior to treatment. In an embodiment, administering a composition comprising an mRNA sequence of the disclosure results in an increase in CFTR levels relative to baseline CFTR levels in the subject prior to treatment. In some embodiments, the increase in CFTR levels can be at least about 5%, 10%, 20%, 30%, 40%, 50%, 100%, 200%, or more.

In embodiments, the CFTR protein is expressed in the lung of a treated subject.

In some embodiments, administering a composition comprising an mRNA sequence of the disclosure results in the expression of a natural, non-mutated human CFTR (i.e., normal or wild-type CFTR as opposed to abnormal or mutated CFTR) protein level at or above about 10 ng/mg, about 20 ng/mg, about 50 ng/mg, about 100 ng/mg, about 150 ng/mg, about 200 ng/mg, about 250 ng/mg, about 300 ng/mg, about 350 ng/mg, about 400 ng/mg, about 450 ng/mg, about 500 ng/mg, about 600 ng/mg, about 700 ng/mg, about 800 ng/mg, about 900 ng/mg, about 1000 ng/mg, about 1200 ng/mg or about 1500 ng/mg of the total protein in the lung epithelial cells of a treated subject.

In some embodiments, the expression of the natural, non-mutated human CFTR protein is detectable 6, 12, 18, 24, 30, 36, 48, 60, and/or 72 hours after administration of a composition comprising an mRNA sequence of the disclosure. In some embodiments, the expression of the natural, non-mutated human CFTR protein is detectable 1 day, 2 days, 3 days, 4 days, 5 days, 6 days, and/or 7 days after administration of a composition comprising an mRNA sequence of the disclosure. In some embodiments, the expression of the natural, non-mutated human CFTR protein is detectable 1 week, 2 weeks, 3 weeks, and/or 4 weeks after the administration. In some embodiments, the expression of the natural, non-mutated human CFTR protein is detectable after administration of a composition comprising an mRNA sequence of the disclosure. In some embodiments, expression of natural, non-mutated human CFTR protein is detectable after administration of a composition comprising an mRNA sequence of the disclosure.

Design and Synthesis of mRNA Sequences

The mRNA agents of the present disclosure may be obtained by any suitable means. Methods for the manufacture of mRNA are known in the art and would be readily apparent to a person of ordinary skill. An mRNA of the present disclosure may be prepared according to any available technique including, but not limited to chemical synthesis, in vitro transcription (IVT) or enzymatic or chemical cleavage of a longer precursor, etc.

In some embodiments, mRNA is produced from a primary complementary DNA (cDNA) construct. The cDNA constructs can be produced on an RNA template by the action of a reverse transcriptase (e.g., RNA-dependent DNA-polymerase). The process of design and synthesis of the primary cDNA constructs described herein generally includes the steps of gene construction, mRNA production (either with or without modifications) and purification. In the IVT method, a target polynucleotide sequence encoding a CFTR protein is first selected for incorporation into a vector, which will be amplified to produce a cDNA template. Optionally, the target polynucleotide sequence and/or any flanking sequences may be codon optimized. The cDNA template is then used to produce mRNA through in vitro transcription (IVT). After production, the mRNA may undergo purification and clean-up processes, the steps of which are provided in more detail below.

The step of gene construction may include, but is not limited to gene synthesis, vector amplification, plasmid purification, plasmid linearization and clean-up, and cDNA template synthesis and clean-up. Once a human CFTR protein (e.g. SEQ ID NOs: 93 or 99) is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding a polypeptide of interest. ORFs often begin with the start codon, ATG and end with a nonsense or termination codon or signal.

The cDNA templates may be transcribed to produce an mRNA sequence described herein using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed mRNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or capping at initiation of in vitro transcription, by for example, including a capping agent as part of the IVT reaction. (Nuc. Acids Symp. (2009) 53:129). A poly-A tailing reaction may be performed by methods known in the art, such as, but not limited to, 2′ O-methyltransferase and by methods as described herein. If the primary construct generated from cDNA does not include a poly-T, it may be beneficial to perform the poly-A-tailing reaction before the primary construct is cleaned.

Codon optimized cDNA constructs encoding a Cystic Fibrosis Transmembrane Conductance Regulator (CFTR) protein are particularly suitable for generating mRNA sequences described herein. For example, such cDNA constructs may be used as the basis to transcribe, in vitro, a polyribonucleotide encoding a Cystic Fibrosis Transmembrane Conductance Regulator (CFTR) protein.

Examples of DNA ORF sequences are provided in SEQ ID Nos: 1-46, which provide sequences which can be used in developing materials for transcription to an mRNA of the present disclosure. SEQ ID NO: 1 provides the DNA ORF of a reference hCFTR protein (construct 764) commonly used in the art as a reference sequence in which the sequence is slightly modified from the wild-type having a point mutation in the coding region to remove an internal cryptic promoter. Preferred DNA ORF sequences include the DNA sequence of SEQ ID NOs: 3, 5, 7, 20, 22, 23, or 26. In some embodiment, the DNA ORF comprises a sequence of SEQ ID NO: 7, which has an optimized coding sequence encoding a CFTR protein of SEQ ID NO: 93. It will be appreciated that T present in DNA is substituted with U in RNA, and vice versa.

The present disclosure also provides expression vectors comprising a nucleotide sequence encoding a CFTR protein that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides polynucleotides (e.g. DNA, RNA, cDNA, mRNA, etc.) encoding a human CFTR protein that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

The present disclosure also provides a host cell transfected with an mRNA or DNA described herein which encodes a CFTR polypeptide described herein. In some embodiments, the human CFTR polypeptide has the sequence of SEQ ID NO: 99. The host cell may be any prokaryotic or eukaryotic cell. For example, a CFTR polypeptide may be expressed in bacterial cells such as E. coli, insect cells (e.g., using a baculovirus expression system), yeast, or mammalian cells. Other suitable host cells are known to those skilled in the art.

The present disclosure also provides a host cell comprising a vector comprising a polynucleotide of SEQ ID NOs: 2-46.

The present disclosure also provides methods of producing a human wild type CFTR protein of SEQ ID NO: 93. For example, a host cell transfected with an expression vector encoding a CFTR protein can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed CFTR proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the CFTR polypeptide.

### Codon Optimization

A polynucleotide sequence encoding a protein can be altered relative to the wild type for the same sequence to select the best combination of codons that code for the amino acids of the protein. For an mRNA, all or a portion of the mRNA, for example, the coding region or open reading frame (ORF), can be optimized with respect to the codons in that region. Codon-optimized sequences can increase protein expression levels (Gustafsson et al., Codon bias and heterologous protein expression. 2004, Trends Biotechnol 22: 346-53) of the encoded proteins while providing other advantages. Optimization of the codons in a sequence will depend on several characteristics of an mRNA construct including high codon adaptation index (CAI), the Low-U method, mRNA secondary structures, cis-regulatory sequences, GC content and many other similar variables. These variables have been shown to correlate with protein expression levels (Villalobos et al., Gene Designer: a synthetic biology tool for constructing artificial DNA segments. 2006, BMC Bioinformatics 7:285). The high CAI (codon adaptation index) method picks a most frequently used synonymous codon for an entire protein coding sequence. The most frequently used codon for each amino acid is deduced from 74,218 protein-coding genes from a human genome. The Low-U method targets only U-containing codons that can be replaced with a synonymous codon with fewer U moieties. If there are a few choices for the replacement, the more frequently used codon will be selected. The remaining codons in the sequence are not changed by the Low-U method. This method may be used in conjunction with the disclosed mRNAs to design coding sequences that are to be synthesized with, for example, 5-methoxyuridine or N¹-methyl pseudouridine. Methods of codon optimization in combination with the use of a modified nucleotide monomer are described in U.S. 2018/0327471, the contents of which are herein incorporated by reference.

In addition, the nucleotide sequence of any region of the mRNA or DNA template may be codon optimized. Codon optimization methods are known in the art and may be useful in efforts to achieve one or more of several goals. These goals include to match codon frequencies in target and host organisms to ensure proper folding, to bias GC nucleotide pair content to increase mRNA stability or reduce secondary structures, to minimize tandem repeat codons or base runs that may impair gene construction or expression, to customize transcriptional and translational control regions, to insert or remove protein trafficking sequences, to remove/add post translation modification sites in encoded protein (e.g. glycosylation sites), to add, remove or shuffle protein domains, to insert or delete restriction sites, to modify ribosome binding sites and mRNA degradation sites, to adjust translational rates to allow the various domains of the protein to fold properly, or to reduce or eliminate problematic secondary structures within the mRNA. Suitable codon optimization tools, algorithms and services are known in the art.

In some embodiments, the nucleotide sequence of any region of the mRNA or DNA templates described herein may be codon-optimized. Preferably, the primary cDNA template may include reducing the occurrence or frequency of appearance of certain nucleotides in the template strand. For example, the occurrence of a nucleotide in a template may be reduced to a level below 25% of said nucleotides in the template. In further examples, the occurrence of a nucleotide in a template may be reduced to a level below 20% of said nucleotides in the template. In some examples, the occurrence of a nucleotide in a template may be reduced to a level below 16% of said nucleotides in the template. Preferably, the occurrence of a nucleotide in a template may be reduced to a level below 15%, and preferably may be reduced to a level below 12% of said nucleotides in the template.

In some embodiments, the nucleotide reduced is uridine. For example, the present disclosure provides nucleic acids with altered uracil content wherein at least one codon in the wild-type sequence has been replaced with an alternative codon to generate a uracil-altered sequence. Altered uracil sequences can have at least one of the following properties:

(i) an increase or decrease in global uracil content (i.e., the percentage of uracil of the total nucleotide content in the nucleic acid of a section of the nucleic acid, e.g., the open reading frame);

(ii) an increase or decrease in local uracil content (i.e., changes in uracil content are limited to specific subsequences);

(iii) a change in uracil distribution without a change in the global uracil content;

(iv) a change in uracil clustering (e.g., number of clusters, location of clusters, or distance between clusters); or

(v) combinations thereof.

In some embodiments, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the nucleic acid sequences of the disclosure. The percentage uracil content can be determined by dividing the number of uracil in a sequence by the total number of nucleotides and multiplying by 100.

In some embodiments, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure. These subsequences can also be part of the wild-type sequences of the heterologous 5′ and 3′ UTR sequences of the present disclosure.

In some embodiments, codons in the nucleic acid sequence of the disclosure reduce or modify, for example, the number, size, location, or distribution of uracil clusters that could have deleterious effects on protein translation. Although lower uracil content is desirable in certain aspects, the uracil content, and in particular the local uracil content, of some subsequences of the wild-type sequence can be greater than the wild-type sequence and still maintain beneficial features (e.g., increased expression).

In some embodiments, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial mRNA contains no nucleoside modifications, whereas mammalian mRNAs have modified nucleosides such as 5-methylcytidine (m⁵C), N⁶-methyladenosine (m⁶A), inosine and many 2′-O-methylated nucleosides in addition to N⁷-methylguanosine (m⁷G).

In some embodiments, the uracil content of polynucleotides disclosed herein and preferably polynucleotides encoding the CFTR protein of SEQ ID NO: 99 is less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the polynucleotide sequence. In some embodiments, the uracil content of polynucleotides disclosed herein and preferably polynucleotides encoding the CFTR protein of SEQ ID NO: 99, is between about 5% and about 25%. In some embodiments, the uracil content of polynucleotides disclosed herein and preferably polynucleotides encoding the CFTR protein of SEQ ID NO: 99 is between about 15% and about 25%.

### Natural, Modified and Chemically-Modified Nucleotides

Preferably an mRNA described herein comprises one or more chemically modified nucleotides. Examples of nucleic acid monomers include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of mRNA polynucleotides comprising chemically modified nucleotides have been shown to improve mRNA expression, expression rates, half-life and/or expressed protein concentrations. Also, mRNA polynucleotides comprising chemically modified nucleotides have been useful in optimizing protein localization, thereby avoiding deleterious bio-responses such as immune responses and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′-O-dimethyl-adenosine, N⁶,N⁶,2′-O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically-modified nucleotides include N¹-alkylguanosines, N²-alkylguanosines, thienoguanosines, 7-deazaguanosines, 8-oxoguanosines, 8-bromoguanosines, O6-alkylguanosines, xanthosines, inosines, and N¹-alkylinosines.

Examples of modified or chemically-modified nucleotides include N¹-methylguanosine, N²-methylguanosine, thienoguanosine, 7-deazaguanosine, 8-oxoguanosine, 8-bromoguanosine, O6-methylguanosine, xanthosine, inosine, and N¹-methylinosine.

Examples of modified or chemically-modified nucleotides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “N1MPU”), N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N⁶-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′-O-methyluridine, 3′-O-dimethyluridine, and 2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenosine, 2-methoxy-adenosine, alpha-thio-adenosine, 2′-O-methyl-adenosine, N⁶,2′-O-dimethyl-adenosine, N⁶,N⁶,2′-O-trimethyl-adenosine, 2′-O-dimethyl-adenosine, 2′-O-ribosyladenosine, 2-amino-N⁶-methyl-purine, 1-thio-adenosine, 2′-fluoro-ara-adenosine, 2′-fluoro-adenosine, 2′-OH-ara-adenosine, and N⁶-(19-amino-pentaoxanonadecyl)-adenosine.

Examples of modified or chemically-modified nucleotides include N¹-alkylguanosines, N²-alkylguanosines, thienoguanosines, 7-deazaguanosines, 8-oxoguanosines, 8-bromoguanosines, O⁶-alkylguanosines, xanthosines, inosines, and N¹-alkylinosines.

Examples of modified or chemically-modified nucleotides include N¹-methylguanosine, N²-methylguanosine, thienoguanosine, 7-deazaguanosine, 8-oxoguanosine, 8-bromoguanosine, O⁶-methylguanosine, xanthosine, inosine, and N¹-methylinosine.

Examples of modified or chemically-modified nucleotides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Other examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “N1MPU”), N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an embodiment, the modified monomer is a locked nucleic acid nucleotide (LNA).

Examples of modified and chemically-modified nucleotide monomers include 2′,4′-constrained 2′-O-methoxy ethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N⁶-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Some further examples of modified nucleotides are given in Saenger, Principles of Nucleic Acid Structure, Springer-Verlag, 1984.

Any of the example base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine.

### 5′ Capping Structure

A Cap structure on the 5′-end of mRNAs, which is present in all eukaryotic organisms (and some viruses) is important for stabilizing mRNAs in vivo. Naturally occurring Cap structures comprise a ribo-guanosine residue that is methylated at position N⁷ of the guanine base. This 7-methylguanosine (m⁷G) is linked via a 5′- to 5′-triphosphate chain at the 5′-end of the mRNA molecule. The presence of the m⁷Gppp fragment on the 5′-end is essential for mRNA maturation as it protects the mRNAs from degradation by exonucleases, facilitates transport of mRNAs from the nucleus to the cytoplasm and plays a key role in assembly of the translation initiation complex (Cell 9:645-653, (1976); Nature 266:235, (1977); Federation of Experimental Biologists Society Letter 96:1-11, (1978); Cell 40:223-24, (1985); Prog. Nuc. Acid Res. 35:173-207, (1988); Ann. Rev. Biochem. 68:913-963, (1999); and J Biol. Chem. 274:30337-3040, (1999)).

Only those mRNAs that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. In some embodiments, the 5′ cap is m⁷GpppAmpG, which is known in the art. In some embodiments, the 5′ cap is m⁷GpppG or m⁷GpppGm, which are known in the art. Structural formulas for embodiments of 5′ cap structures are provided below.

In some embodiments, an mRNA described herein comprises a 5′ cap having the structure of Formula (Cap I).

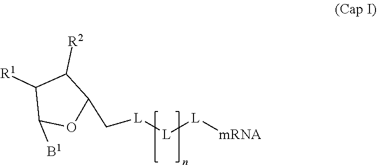

wherein B¹ is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1; and mRNA represents an mRNA of the present disclosure linked at its 5′ end. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, an mRNA described herein comprises a 5′ cap having the structure of Formula (Cap II).

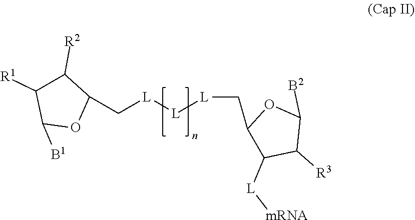

wherein B¹ and B² are each independently a natural or modified nucleobase; R¹, R², and R³ are each independently selected from a halogen, OH, and OCH₃; each L is independently selected from the group consisting of phosphate, phophorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, an mRNA described herein comprises a 5′ cap having the structure of Formula (Cap III).

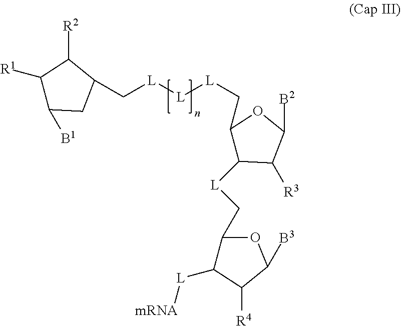

wherein B¹, B², and B³ are each independently a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, an mRNA described herein comprises a m⁷GpppG 5′ cap analog having the structure of Formula (Cap IV).

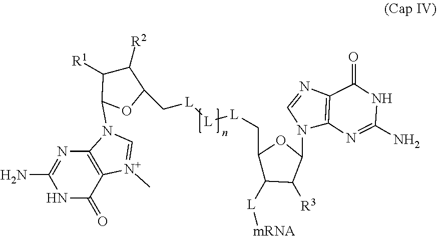

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m7GpppGm, wherein R¹ and R² are each OH, R³ is OCH₃, each L is a phosphate, mRNA is a CFTR mRNA of the present disclosure linked at its 5′ end, and n is 1.

In some embodiments, an mRNA described herein comprises a m⁷GpppAmpG 5′ cap analog having the structure of Formula (Cap V).

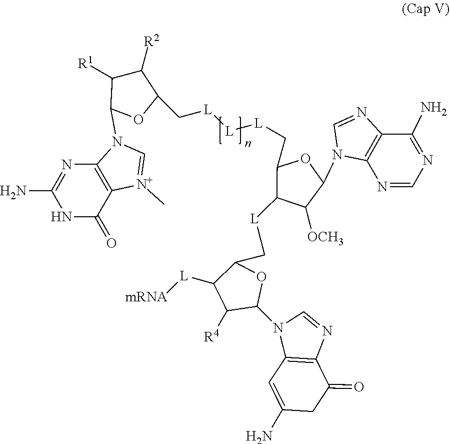

wherein, R¹, R², and R⁴ are each independently selected from a halogen, OH, and OCH₃; each L is independently selected from the group consisting of a phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, the compound of Formula Cap V is m⁷GpppAmpG, wherein R¹, R², and R⁴ are each OH, n is 1, and each L is a phosphate. In some embodiments, n is 1.

### 3′ Tail

Polyadenylation is the addition of a poly-A tail, a chain of adenine nucleotides usually about 100-120 monomers in length, to an mRNA. In eukaryotes, polyadenylation is part of the process that produces mature mRNA for translation and begins as the transcription of a gene terminates. The 3′-most segment of a newly made pre-mRNA is first cleaved off by a set of proteins; these proteins then synthesize the poly-A tail at the 3′ end. The poly-A tail is important for the nuclear export, translation, and stability of mRNA. The tail is shortened over time, and, when it is short enough, the mRNA is enzymatically degraded. However, in a few cell types, mRNAs with short poly-A tails are stored for later activation by re-polyadenylation in the cytosol.

Poly-A tails can be added using a variety of methods known in the art, e.g., using poly-A polymerase to add tails to synthetic or in vitro transcribed RNA. Other methods include the use of a transcription vector to encode poly-A tails or the use of a ligase (e.g., via splint ligation using a T4 RNA ligase and/or T4 DNA ligase), wherein poly-A may be ligated to the 3′ end of a RNA. In some embodiments, a combination of any of the above methods is utilized.

In some embodiments, the mRNA sequence encoding CFTR comprises a tail region, which can serve to protect the mRNA from exonuclease degradation. In some embodiments, the tail region can be a poly-A tail. The tail region may be a 3′ poly-A and/or 3′ poly-C region. Preferably, the tail region is a 3′ poly-A tail. As used herein a “3′ poly-A tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; 112-121 sequential adenine nucleotides; 114-121 sequential adenine nucleotides; or 115 to 121 sequential adenine nucleotides. Preferably, a 3′ poly-A tail as described herein comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides.

In some embodiments, an mRNA sequence comprises a 3′ poly-A tail structure. In some embodiments, the length of the poly-A tail can be at least about 5, 10, 15, 20, 25, 30, 35, 40, 45, 50, 100, 200, or 300 nucleotides. In some embodiments, a 3′ poly-A tail contains about 5 to 300 adenosine nucleotides (e.g., about 30 to 250 adenosine nucleotides, about 60 to 220 adenosine nucleotides, about 80 to 200 adenosine nucleotides, about 90 to about 150 adenosine nucleotides, or about 100 to about 120 adenosine nucleotides). In an embodiment, the 3′ poly-A tail is about 100 nucleotides in length. In another embodiment, the 3′ poly-A tail is about 115 nucleotides in length. In another embodiment, the 3′ poly-A tail is about 250 nucleotides in length.

In some embodiments, the 3′ poly-A tail comprises one or more UNA monomers. In some embodiments, the 3′ poly-A tail contains 2, 3, 4, 5, 10, 15, 20, or more UNA monomers. In an embodiment, the 3′ poly-A tail contains 2 UNA monomers. In a further embodiment, the 3′ poly-A tail contains 2 UNA monomers which are found consecutively, i.e., contiguous to each other in the 3′ poly-A tail. Synthetic methods and example constructs for UNA-containing poly-A tails are described in WO 2016/070166, the contents of which are incorporated herein by reference.

In an embodiment, the 3′ poly-A tail comprises a sequence of Poly-A100 or Poly-A120, which consist of 100 or 120 adenosine nucleotides,

In some embodiments, the mRNA sequence comprises a 3′ poly-C tail structure. In some embodiments, the length of the poly-C tail can be at least about 5, 10, 15, 20, 25, 30, 35, 40, 45, 50, 100, 200, or 300 nucleotides. In some embodiments, a 3′ poly-C tail contains about 5 to 300 cytosine nucleotides (e.g., about 30 to 250 cytosine nucleotides, about 60 to 220 cytosine nucleotides, about 80 to about 200 cytosine nucleotides, about 90 to 150 cytosine nucleotides, or about 100 to about 120 cytosine nucleotides). In an embodiment, the 3′ poly-C tail is about 100 nucleotides in length. In another embodiment, the 3′ poly-C tail is about 115 nucleotides in length. The poly-C tail may be added to the poly-A tail or may substitute the poly-A tail. The poly-C tail may be added to the 5′ end of the poly-A tail or the 3′ end of the poly-A tail.

In some embodiments, the length of the poly-A and/or poly-C tail is adjusted to control the stability of a modified mRNA of the disclosure and, thus, the transcription of protein. For example, since the length of the poly-A tail can influence the half-life of an mRNA sequence, the length of the poly-A tail can be adjusted to modify the level of resistance of the mRNA to nucleases and thereby control the time course of polynucleotide expression and/or polypeptide production in a target cell.

### 5′ and 3′ Untranslated Regions (UTRs)

In molecular genetics, an untranslated region (UTR) refers to either of two sections, one on each side of a coding sequence on a strand of mRNA. If it is found on the 5′ side, it is called the 5′ UTR (or leader sequence), or if it is found on the 3′ side, it is called the 3′ UTR (or trailer sequence). As a mRNA is translated into a protein in vivo, several regions of the mRNA are usually not translated, including the 5′ and 3′ UTRs. In some embodiments, an mRNA described herein further comprises a 5′ untranslated region (UTR) sequence. The 5′ UTR is upstream from the coding sequence. Within the 5′ UTR is a sequence that is recognized by the ribosome which allows the ribosome to bind and initiate translation. In contrast, the 3′ UTR is typically found immediately following the translation stop codon of the coding region. The 3′ UTR can play an important role in translation termination as well as post-transcriptional modification. Thus, as is understood in the art, the 5′ and/or 3′ UTR may affect an mRNA's stability or efficiency of translation. The 5′ UTR may be derived from an mRNA molecule known in the art as relatively stable (e.g., histone, tubulin, globin, glyceraldehyde 1-phosphate dehydrogenase (GAPDH), actin, or citric acid cycle enzymes) to increase the stability of the translatable oligomer. In other embodiments, a 5′ UTR sequence may include a partial sequence of a cytomegalovirus (CMV) immediate-early 1 (IE1) gene.

In some embodiments, the mRNA sequence may comprise a 5′ UTR that is at least about 25, 50, 75, 100, 125, 150, 175, 200, 300, 400, or 500 nucleotides. In some embodiments, a 5′ UTR contains about 50 to 300 nucleotides (e.g., about 75 to 250 nucleotides, about 100 to 200 nucleotides, about 120 to 150 nucleotides, or about 135 nucleotides). In an embodiment, the 5′ UTR is about 127 nucleotides in length.

Preferably, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). Preferably, an mRNA described herein comprises a 5′ UTR sequence that is derived from a gene expressed by Arabidopsis thaliana. Preferably, the 5′ UTR sequence of a gene expressed by Arabidopsis thaliana is AT1G58420. Examples of 5′ UTRs and 3′ UTRs are described in WO 2018/222890, the contents of which are herein incorporated by reference. Preferred 5′ UTR sequences comprise a sequence selected from SEQ ID NOs: 106-125.

In some embodiments, the 5′ UTR sequence comprises SEQ ID NO: 106 (TEV). In some embodiments, the 5′ UTR sequence comprises SEQ ID NO: 107 (AT1G58420).

In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. In some embodiments, the 3′ UTR is derived from Xenopus beta globin. Examples of 3′ UTR sequences include SEQ ID NOs: 126-145. In some embodiments, the 3′ UTR sequence comprises SEQ ID NO: 126 (XBG).

In certain embodiments, the mRNA sequence encoding CFTR comprises a 5′ UTR sequence of SEQ ID NOs: 106-125 and a 3′ UTR sequence selected from SEQ ID NOs: 126-145. In some embodiments, the 5′ UTR sequence comprises SEQ ID NO: 106 and the 3′ UTR sequence comprises SEQ ID NO: 126.

### Triple Stop Codon

In some embodiments, the translatable oligomer or polymer encoding CFTR may comprise a sequence immediately downstream of a coding region (i.e., ORF) that creates a triple stop codon. A triple stop codon is a sequence of three consecutive stop codons. The triple stop codon can ensure total insulation of an expression cassette and may be incorporated to enhance the efficiency of translation. In some embodiments, the mRNA may comprise the sequence UAG, UGA, or UAA immediately downstream of an ORF described herein. The triple combination can be three of the same codons, three different codons, or any other permutation of the three stop codons.

### Translation Enhancers and Kozak Sequences

For translation initiation, proper interactions between ribosomes and mRNAs must be established to determine the exact position of the translation initiation region. However, ribosomes also must dissociate from the translation initiation region to slide toward the downstream sequence during mRNA translation. Translation enhancers upstream from initiation sequences of mRNAs enhance the yields of protein biosynthesis. Several studies have investigated the effects of translation enhancers. In some embodiments, an mRNA described herein comprises a translation enhancer sequence. These translation enhancer sequences enhance the translation efficiency of a mRNA described herein and thereby provide increased production of the protein encoded by the mRNA. The translation enhancer region may be located in the 5′ or 3′ UTR of an mRNA sequence. Examples of translation enhancer regions include naturally occurring enhancer regions from the TEV 5′ UTR and the Xenopus beta-globin 3′ UTR. Example 5′ UTR enhancer sequences include but are not limited to those derived from mRNAs encoding human heat shock proteins (HSP) including HSP70-P2, HSP70-M1 HSP72-M2, HSP17.9 and HSP70-P1.

In some embodiments, the mRNA sequence encoding CFTR may comprise a Kozak sequence. As is understood in the art, a Kozak sequence is a short consensus sequence centered around the translational initiation site of eukaryotic mRNAs that allows for efficient initiation of translation of the mRNA. See, for example, Kozak, Marilyn (1988) Mol. and Cell Biol, 8:2737-2744; Kozak, Marilyn (1991) J. Biol. Chem, 266: 19867-19870; Kozak, Marilyn (1990) Proc Natl. Acad. Sci. USA, 87:8301-8305; and Kozak, Marilyn (1989) J. Cell Biol, 108:229-241; and the references cited therein. It ensures that a protein is correctly translated from the genetic message, mediating ribosome assembly and translation initiation. The ribosomal translation machinery recognizes the AUG initiation codon in the context of the Kozak sequence.

In some embodiments, the translation initiation site (e.g., a Kozak sequence) is inserted upstream of the coding sequence for CFTR. In some embodiments, the translation initiation site is inserted downstream of a 5′ UTR. In certain embodiments, the translation initiation site is inserted upstream of the coding sequence for CFTR and downstream of a 5′ UTR.

As is understood in the art, the length of the Kozak sequence may vary. Generally, increasing the length of the leader sequence enhances translation. In some embodiments, the Kozak sequence is immediately downstream of a 5′ UTR and immediately upstream of the coding sequence for CFTR. In this aspect, Table 1 lists mRNA constructs exemplified herein.

TABLE 1
(mRNA Constructs)

| mRNA | | | | | | | mRNA |
|---|---|---|---|---|---|---|---|
| Construct | | | | CFTR Protein | | 3' Poly A | Construct SEQ |
| No. | Cap | 5' UTR | Kozak | Encoded | 3' UTR | Tail | ID NO: |
| 139 | Capl | TEV | Yes | SEQ ID NO: 93 | XBG | Yes | 47 |
| 140 | Capl | TEV | Yes | SEQ ID NO: 93 | XBG | Yes | 48 |
| 141 | Capl | TEV | Yes | SEQ ID NO: 93 | XBG | Yes | 49 |
| 142 | Capl | TEV | Yes | SEQ ID NO: 93 | XBG | Yes | 50 |
| 143 | Capl | TEV | Yes | SEQ ID NO: 93 | XBG | Yes | 51 |
| 144 | Capl | TEV | Yes | SEQ ID NO: 93 | XBG | Yes | 52 |
| 145 | Capl | TEV | Yes | SEQ ID NO: 93 | XBG | Yes | 53 |
| 146 | Capl | TEV | Yes | SEQ ID NO: 93 | XBG | Yes | 54 |
| 147 | Capl | TEV | No | SEQ ID NO: 93 | XBG | Yes | 55 |
| 148 | Capl | TEV | No | SEQ ID NO: 93 | XBG | Yes | 56 |
| 149 | Capl | TEV | No | SEQ ID NO: 93 | XBG | Yes | 57 |
| 150 | Capl | TEV | Yes | SEQ ID NO: 93 | XBG | Yes | 58 |
| 151 | Capl | TEV | Yes | SEQ ID NO: 93 | XBG | Yes | 59 |
| 152 | Capl | TEV | Yes | SEQ ID NO: 93 | XBG | Yes | 60 |
| 153 | Capl | TEV | Yes | SEQ ID NO: 93 | XBG | Yes | 61 |
| 154 | Capl | TEV | Yes | SEQ ID NO: 93 | XBG | Yes | 62 |
| 155 | Capl | TEV | Yes | SEQ ID NO: 93 | XBG | Yes | 63 |
| 156 | Capl | TEV | Yes | SEQ ID NO: 93 | XBG | Yes | 64 |
| 157 | Capl | TEV | Yes | SEQ ID NO: 93 | XBG | Yes | 65 |
| 158 | Capl | TEV | Yes | SEQ ID NO: 93 | XBG | Yes | 66 |
| 159 | Capl | TEV | Yes | SEQ ID NO: 93 | XBG | Yes | 67 |
| 160 | Capl | TEV | Yes | SEQ ID NO: 93 | XBG | Yes | 68 |
| 161 | Capl | TEV | Yes | SEQ ID NO: 93 | XBG | Yes | 69 |
| 162 | Capl | TEV | Yes | SEQ ID NO: 93 | XBG | Yes | 70 |
| 163 | Capl | TEV | Yes | SEQ ID NO: 93 | XBG | Yes | 71 |
| 164 | Capl | TEV | Yes | SEQ ID NO: 93 | XBG | Yes | 72 |
| 165 | Capl | TEV | Yes | SEQ ID NO: 93 | XBG | Yes | 73 |
| 166 | Capl | TEV | Yes | SEQ ID NO: 93 | XBG | Yes | 74 |
| 167 | Capl | TEV | Yes | SEQ ID NO: 93 | XBG | Yes | 75 |
| 168 | Capl | TEV | Yes | SEQ ID NO: 93 | XBG | Yes | 76 |
| 169 | Capl | TEV | Yes | SEQ ID NO: 93 | XBG | Yes | 77 |
| 170 | Capl | TEV | Yes | SEQ ID NO: 93 | XBG | Yes | 78 |
| 171 | Capl | TEV | Yes | SEQ ID NO: 93 | XBG | Yes | 79 |
| 172 | Capl | TEV | Yes | SEQ ID NO: 93 | XBG | Yes | 80 |
| 173 | Capl | TEV | Yes | SEQ ID NO: 93 | XBG | Yes | 81 |
| 174 | Capl | TEV | Yes | SEQ ID NO: 93 | XBG | Yes | 82 |
| 175 | Capl | TEV | Yes | SEQ ID NO: 93 | XBG | Yes | 83 |
| 176 | Capl | TEV | Yes | SEQ ID NO: 93 | XBG | Yes | 84 |
| 177 | Capl | TEV | Yes | SEQ ID NO: 93 | XBG | Yes | 85 |
| 178 | Capl | TEV | Yes | SEQ ID NO: 93 | XBG | Yes | 86 |
| 179 | Capl | TEV | Yes | SEQ ID NO: 93 | XBG | Yes | 87 |
| 180 | Capl | TEV | Yes | SEQ ID NO: 93 | XBG | Yes | 88 |
| 181 | Capl | TEV | Yes | SEQ ID NO: 93 | XBG | Yes | 89 |
| 182 | Capl | TEV | Yes | SEQ ID NO: 93 | XBG | Yes | 90 |
| 183 | Capl | TEV | Yes | SEQ ID NO: 93 | XBG | Yes | 91 |
| 184 | Capl | TEV | Yes | SEQ ID NO: 93 | XBG | Yes | 92 |
| 766 | Capl | TEV | Yes | SEQ ID NO: 93 | XBG | Yes | 48 |
| | | | | with | | | |
| | | | | modifications | | | |
| 1831 | Capl | TEV | Yes | SEQ ID NO: 99 | XBG | Yes | 49 |
| 1833 | Capl | TEV | Yes | SEQ ID NO: 99 | XBG | Yes | 51 |
| 1835 | Capl | TEV | Yes | SEQ ID NO: 99 | XBG | Yes | 53 |
| 2093 | Capl | TEV | Yes | SEQ ID NO: 99 | XBG | Yes | 66 |
| 2095 | Capl | TEV | Yes | SEQ ID NO: 99 | XBG | Yes | 68 |
| 2096 | Capl | TEV | Yes | SEQ ID NO: 99 | XBG | Yes | 69 |
| 2099 | Capl | TEV | Yes | SEQ ID NO: 99 | XBG | Yes | 72 |

### Lipid-Based Formulations

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), and release at the cytoplasm (for RNA). Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still undergoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and are within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicles (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int. J. Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid is contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed hereinbelow.

### Livid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell can be formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine© reagent, have found considerable utility for in vitro transfection. However, these first-generation lipoplexes have not proven useful in vivo. The large particle size and positive charge (imparted by the cationic lipid) result in rapid plasma clearance, hemolytic and other toxicities, as well as immune system activation.

### Lipid-mRNA Formulations

An mRNA as disclosed herein or a pharmaceutically acceptable salt thereof can be incorporated into a lipid formulation (i.e., a lipid-based delivery vehicle).

In the context of the present disclosure, a lipid-based delivery vehicle typically serves to transport a desired mRNA to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing an mRNA of the present disclosure. In some embodiments, the lipid-based delivery vehicle comprises a nanoparticle or a bilayer of lipid molecules and an mRNA of the present disclosure. In some embodiments, the lipid bilayer preferably further comprises a neutral lipid or a polymer. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic mRNA molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the mRNA is fully encapsulated within the lipid portion of the lipid formulation such that the mRNA in the lipid formulation is resistant in aqueous solution to nuclease degradation. In other embodiments, the lipid formulations described herein are substantially non-toxic to mammals such as humans.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1. In some preferred embodiments, the total lipid:RNA ratio (mass/mass ratio) is from about 10:1 to about 25:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise an mRNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol.

In the nucleic acid-lipid formulations, the mRNA may be fully encapsulated within the lipid portion of the formulation, thereby protecting the nucleic acid from nuclease degradation. In preferred embodiments, a lipid formulation comprising an mRNA is fully encapsulated within the lipid portion of the lipid formulation, thereby protecting the nucleic acid from nuclease degradation. In certain instances, the mRNA in the lipid formulation is not substantially degraded after exposure of the particle to a nuclease at 37° C. for at least 20, 30, 45, or 60 minutes. In certain other instances, the mRNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the mRNA is complexed with the lipid portion of the formulation.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I₀−I)/I₀, where I and I₀ refer to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of mRNA-liposomes. In some embodiments, the nucleic acid-lipid composition comprises a plurality of mRNA-cationic liposomes. In some embodiments, the nucleic acid-lipid composition comprises a plurality of mRNA-lipid nanoparticles.

In some embodiments, the lipid formulations comprise mRNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the mRNA encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, the expressible polynucleotides and mRNA constructs described herein are lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) an mRNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In one some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a PEG-lipid, in a molar ratio of about 20% to about 40% ionizable cationic lipid: about 25% to about 45% helper lipid: about 25% to about 45% sterol; about 0.5-5% PEG-lipid. Example cationic lipids (including ionizable cationic lipids), helper lipids (e.g., neutral lipids), sterols, and ligand-containing lipids (e.g., PEG-lipids) are described hereinbelow.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I.

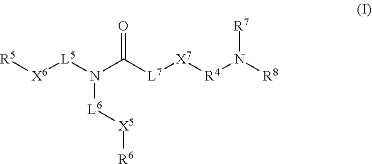

or a pharmaceutically acceptable salt or solvate thereof, wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, X⁷ is S.

In some embodiments, X⁵ is —C(O)O—, whereby —C(O)O—R⁶ is formed and X⁶ is —C(O)O— whereby —C(O)O—R⁵ is formed.

In some embodiments, R⁷ and R⁸ are each independently selected from the group consisting of methyl, ethyl and isopropyl.

In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl.

In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C₃ alkyl. In some embodiments, R⁵ consists of —CH((CH₂)ₚCH₃)((CH₂)ₚ₋₁CH₃), wherein p is 7 or 8.

In some embodiments, R⁴ is ethylene or propylene. In some embodiments, R⁴ is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X^{S} is —C(O)O—, whereby —C(O)O—R⁶ is formed, X⁶ is —C(O)O— whereby —C(O)O—R⁵ is formed, L⁵ and L⁶ are each independently a linear C₃-C₇ alkyl, L⁷ is absent, R⁵ is —CH((CH₂)ₚCH₃)₂, and R⁶ is C₇-C₁₂ alkenyl. In some further embodiments, p is 6 and R⁶ is C₉ alkenyl.

In some embodiments, the lipid formulation comprises an ionizable cationic lipid selected from the group consisting of

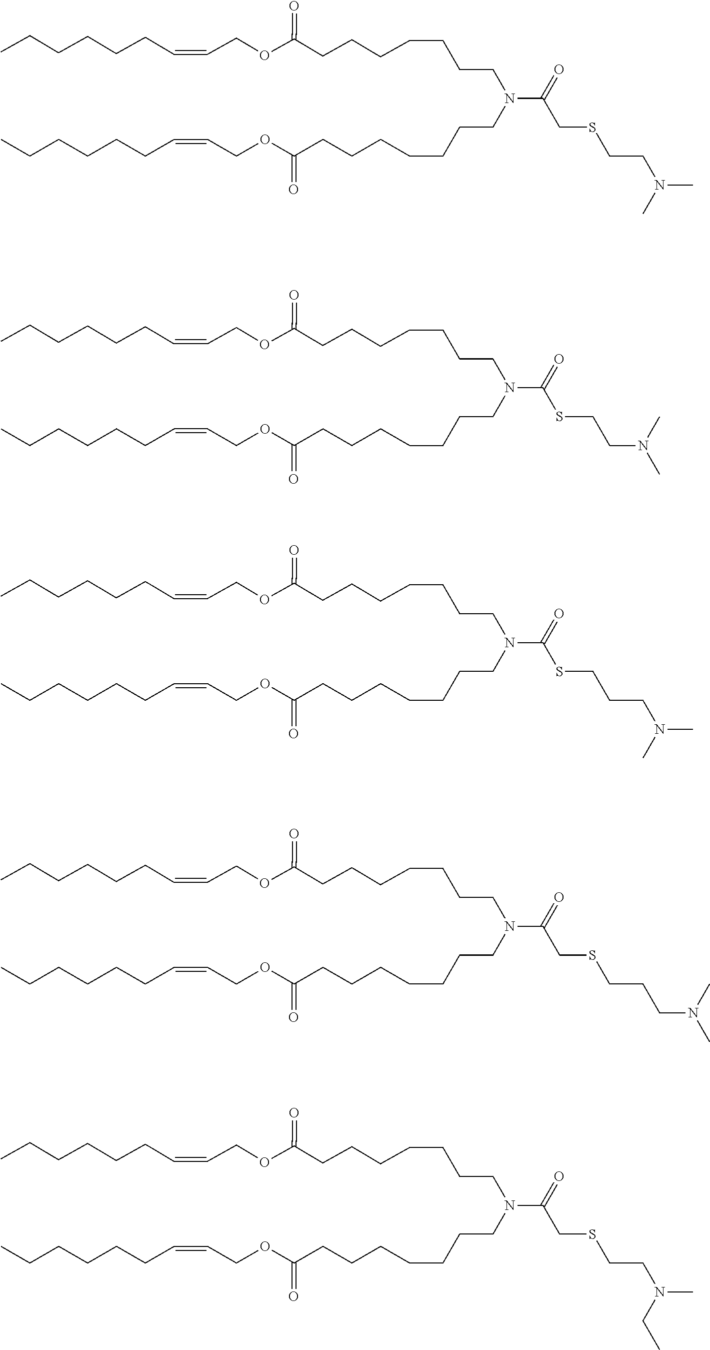

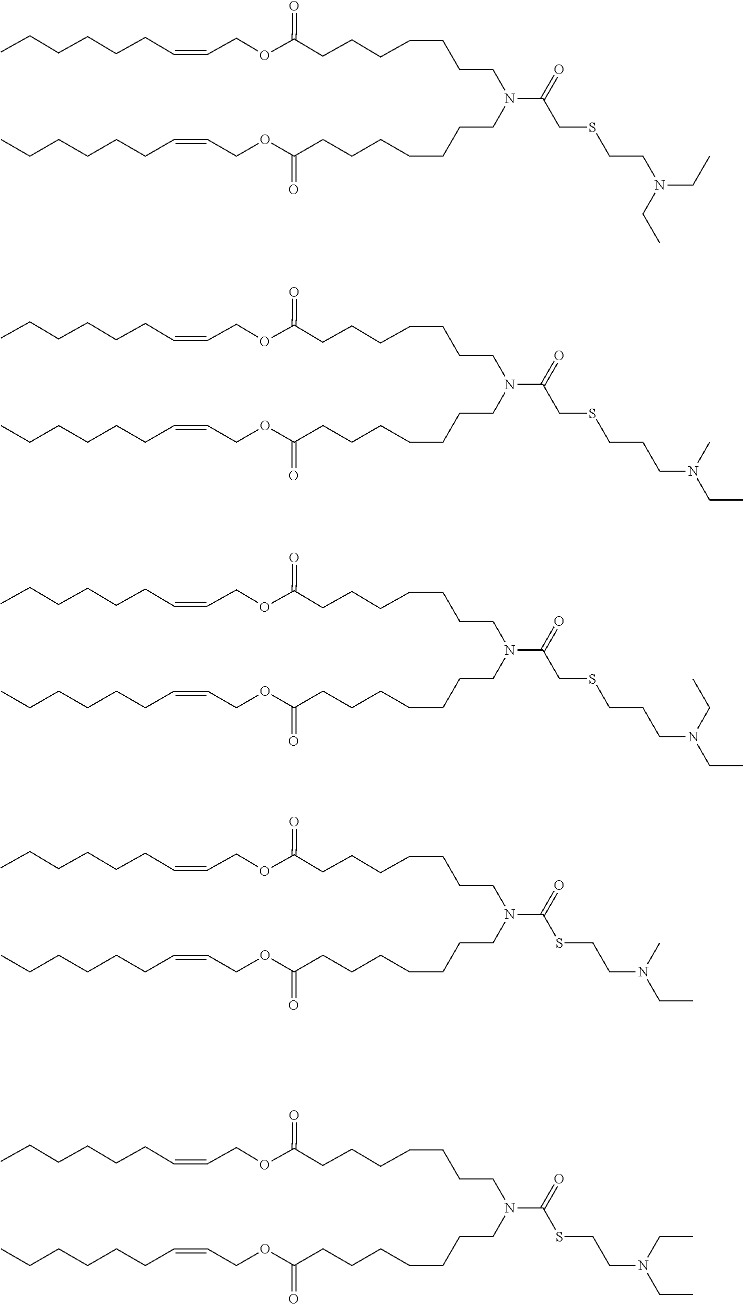

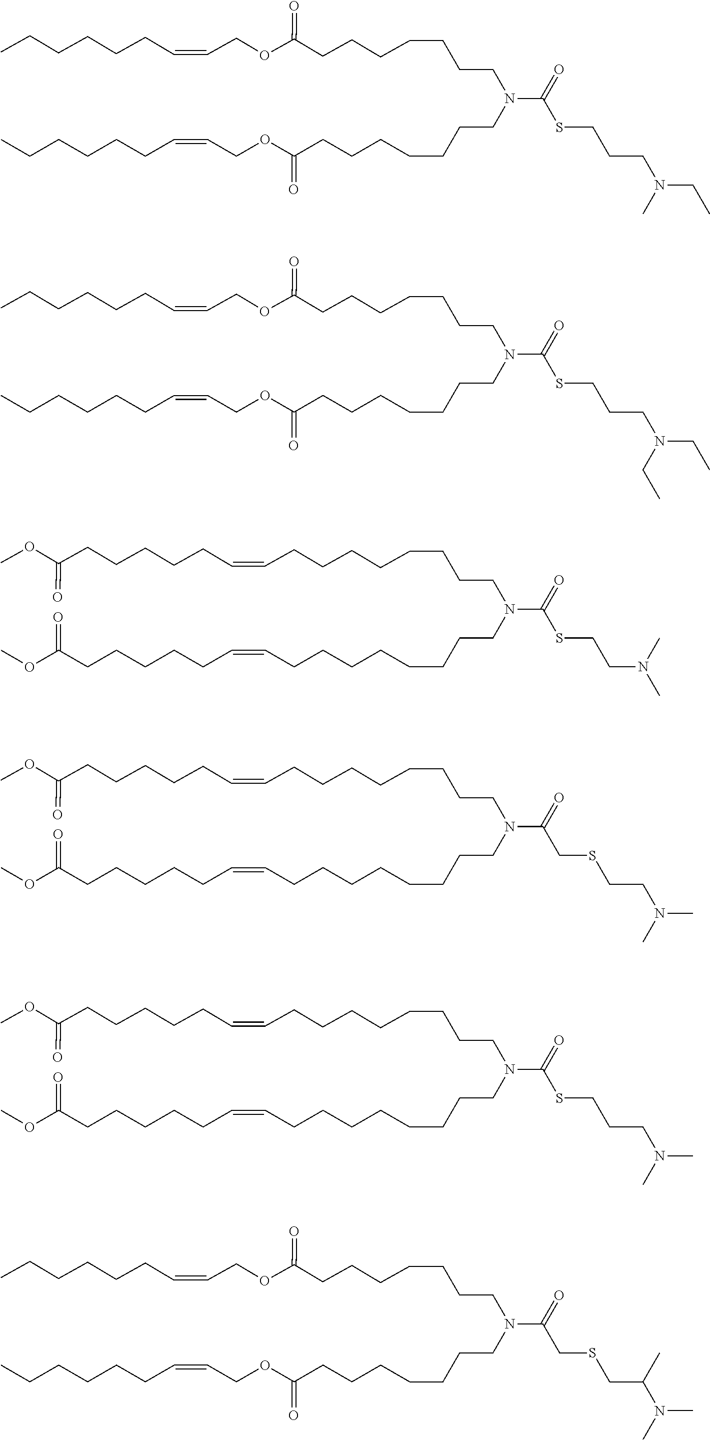

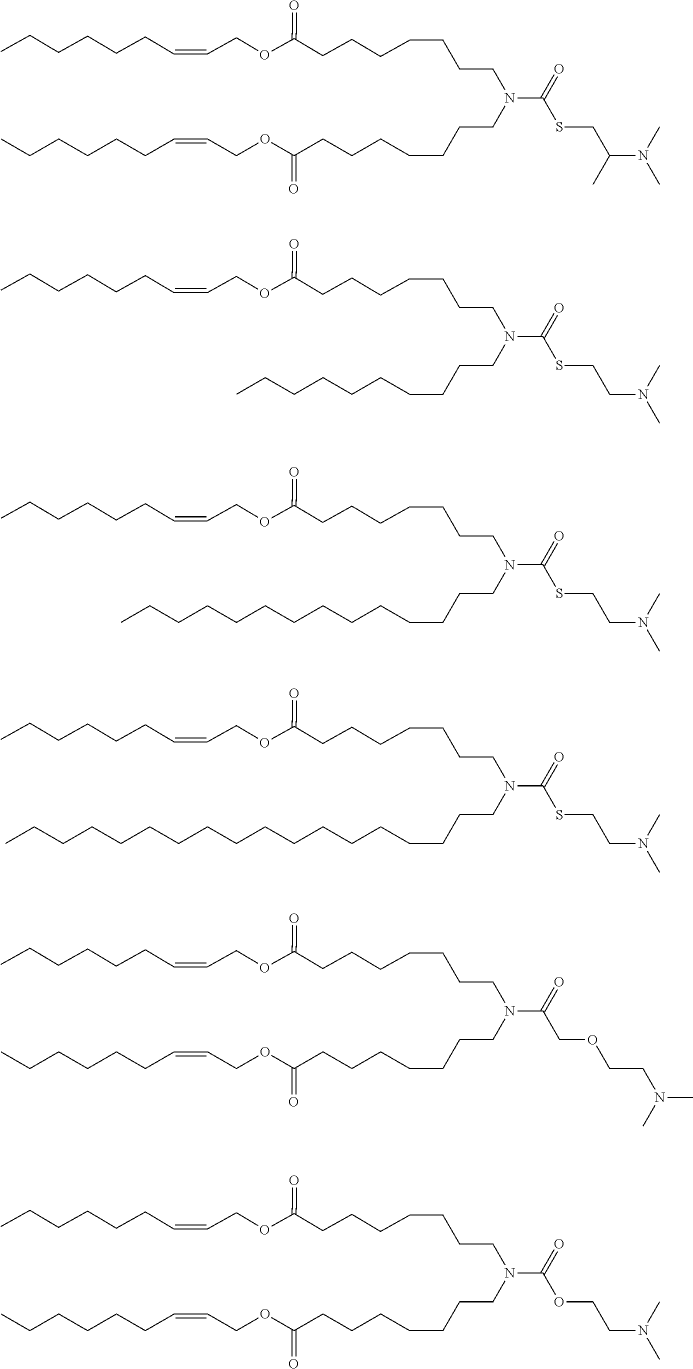

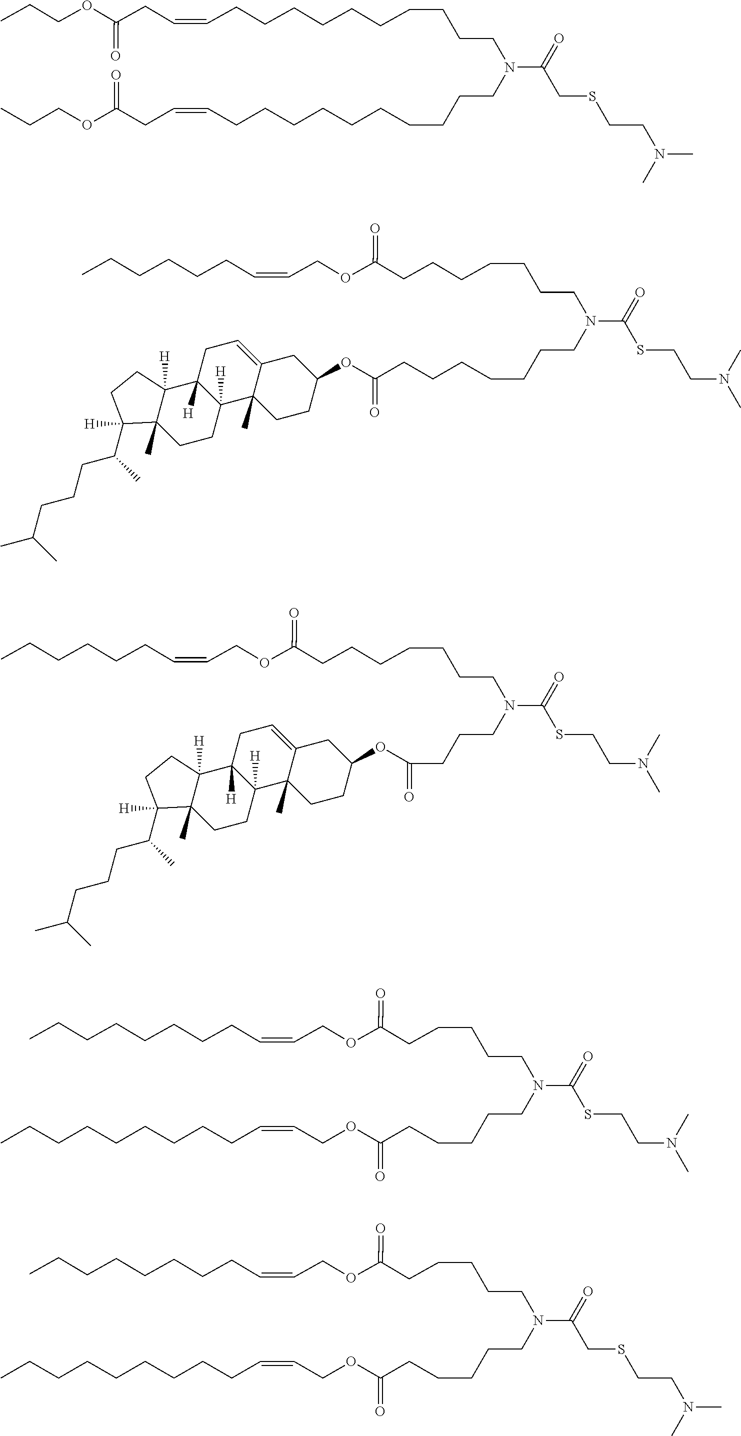

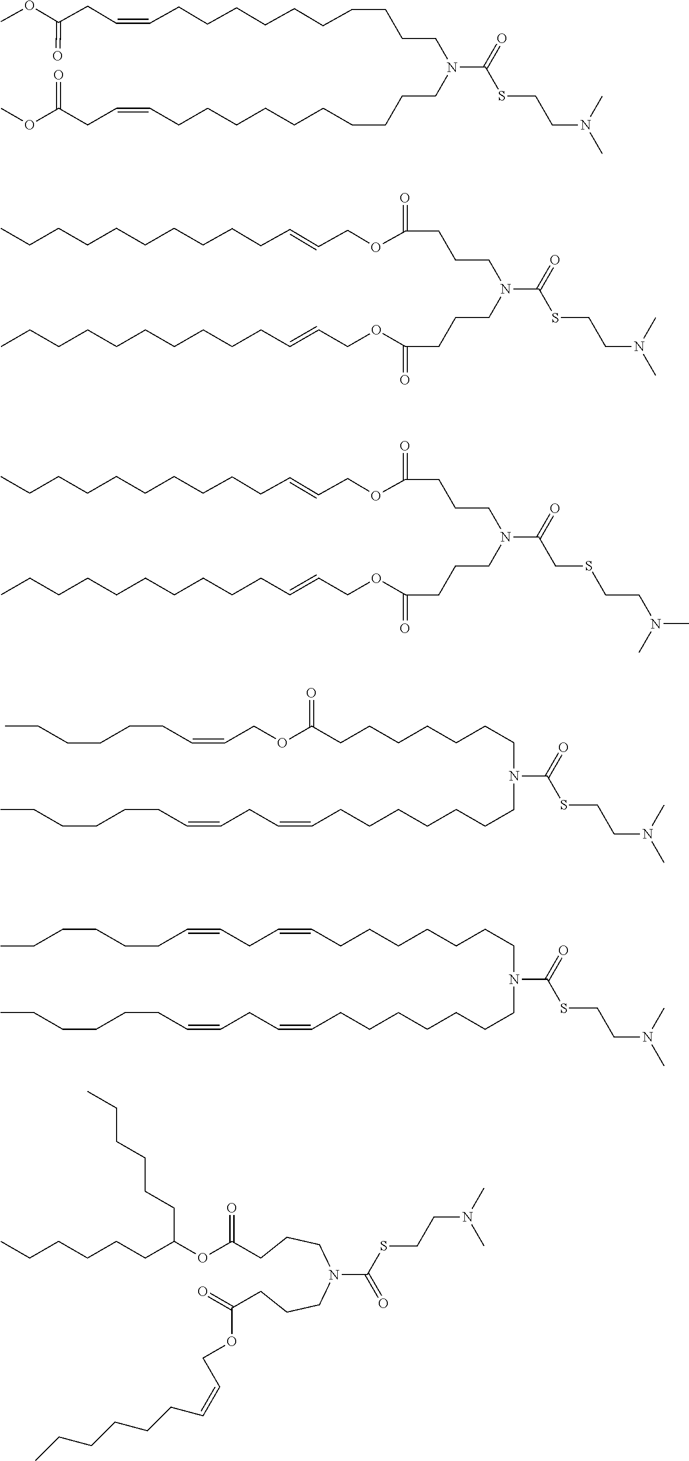

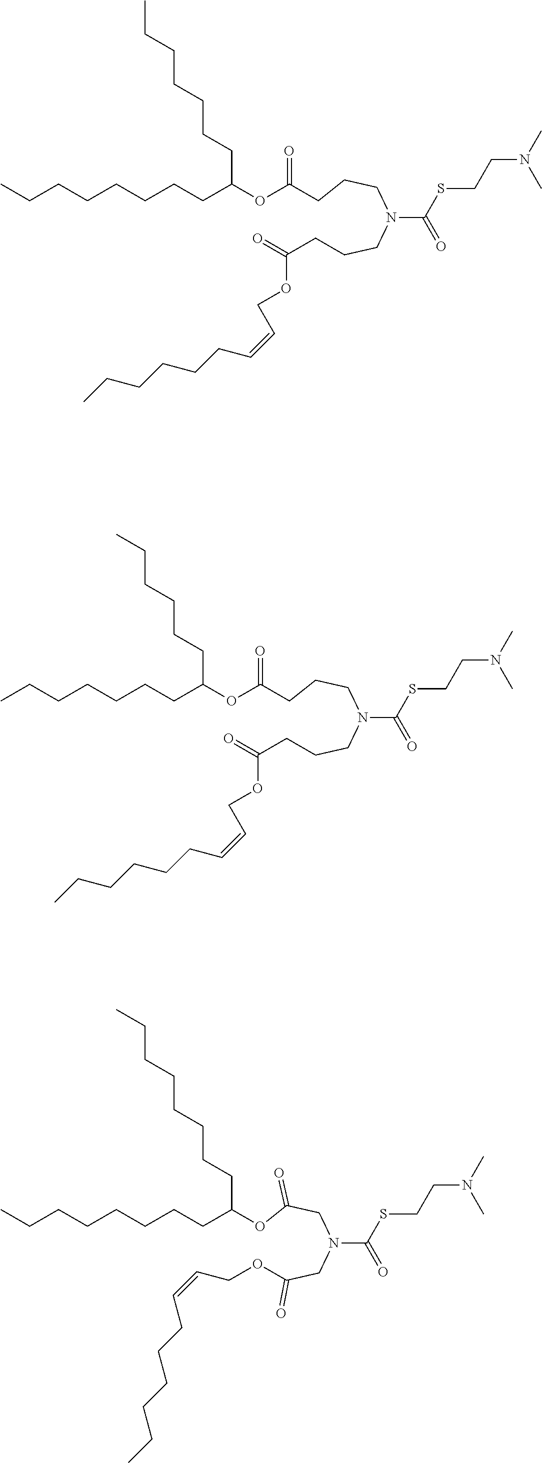

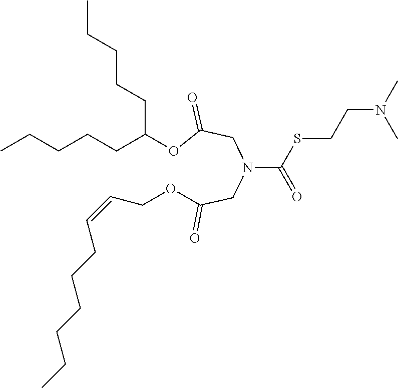 [SMILES: CCCCCC/C=C\COC(=O)CN(CC(=O)OC(CCCCC)CCCCC)C(=O)SCCN(C)C]

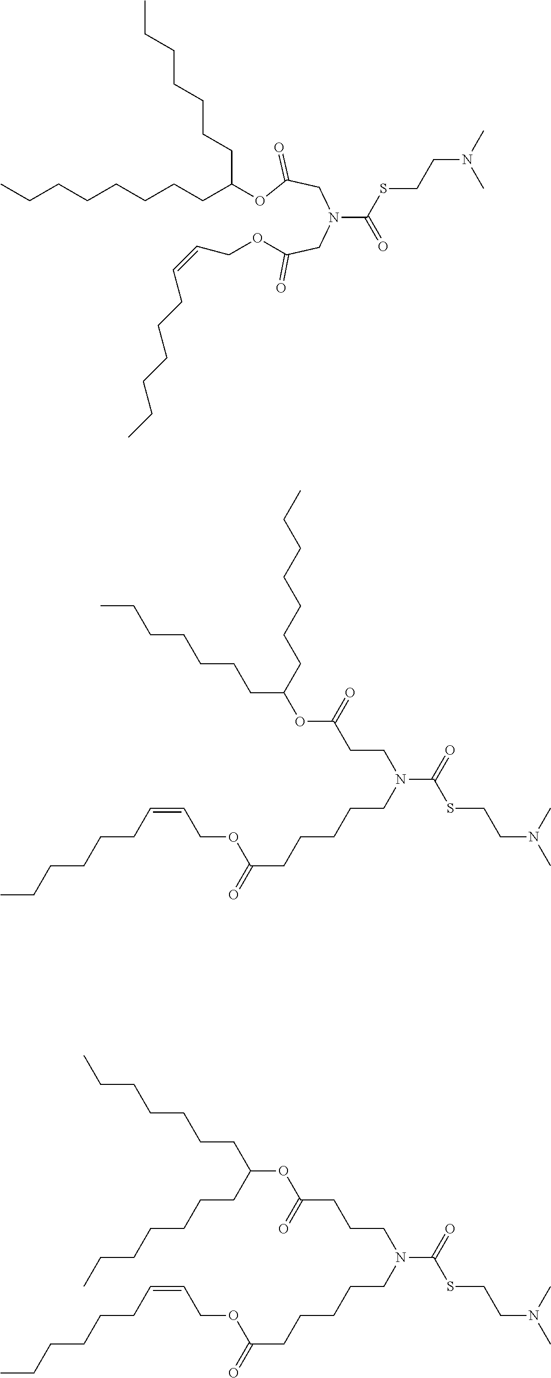

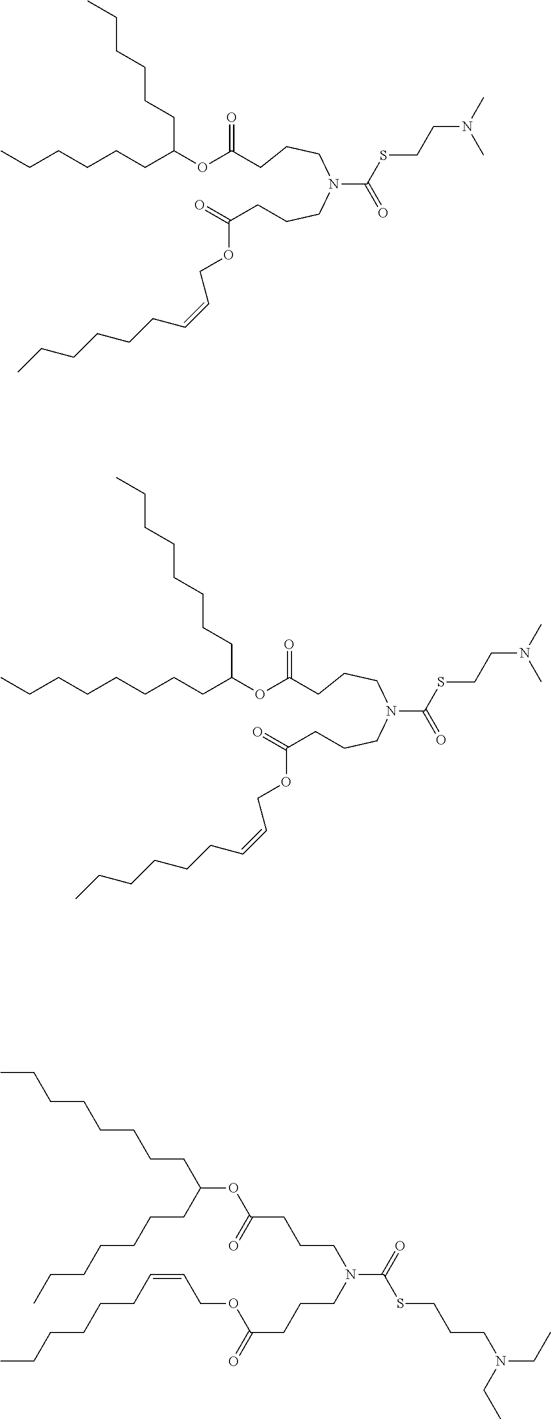

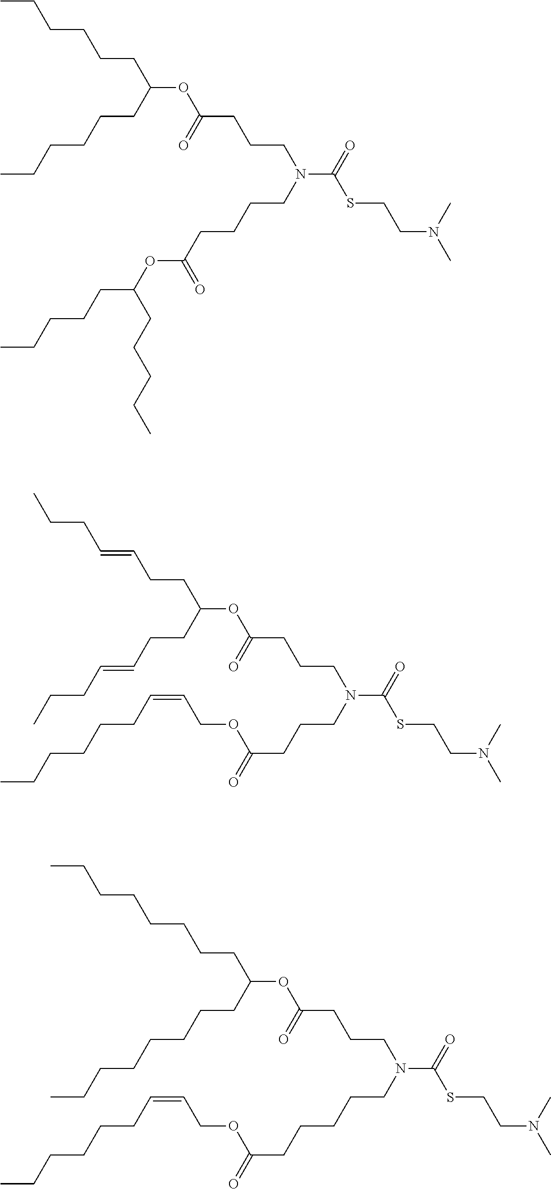

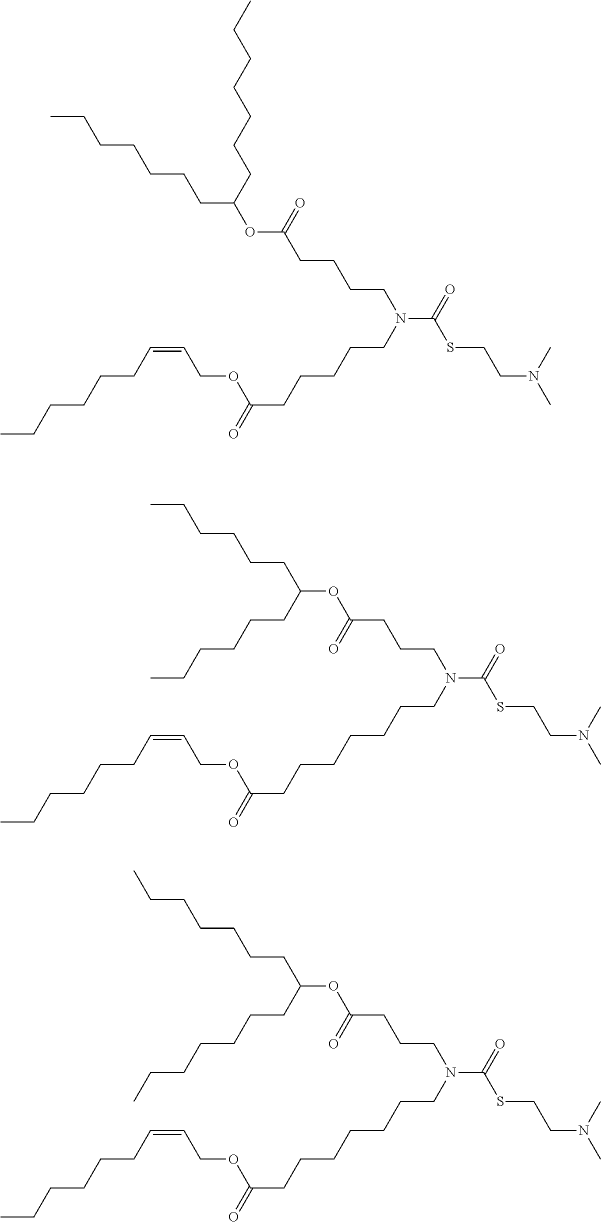

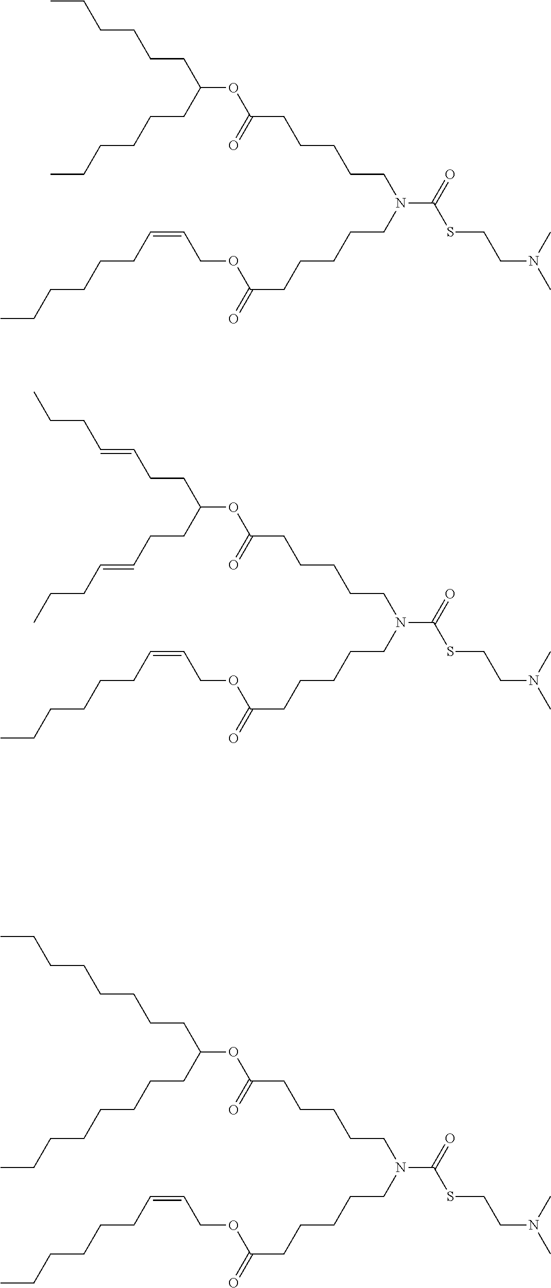

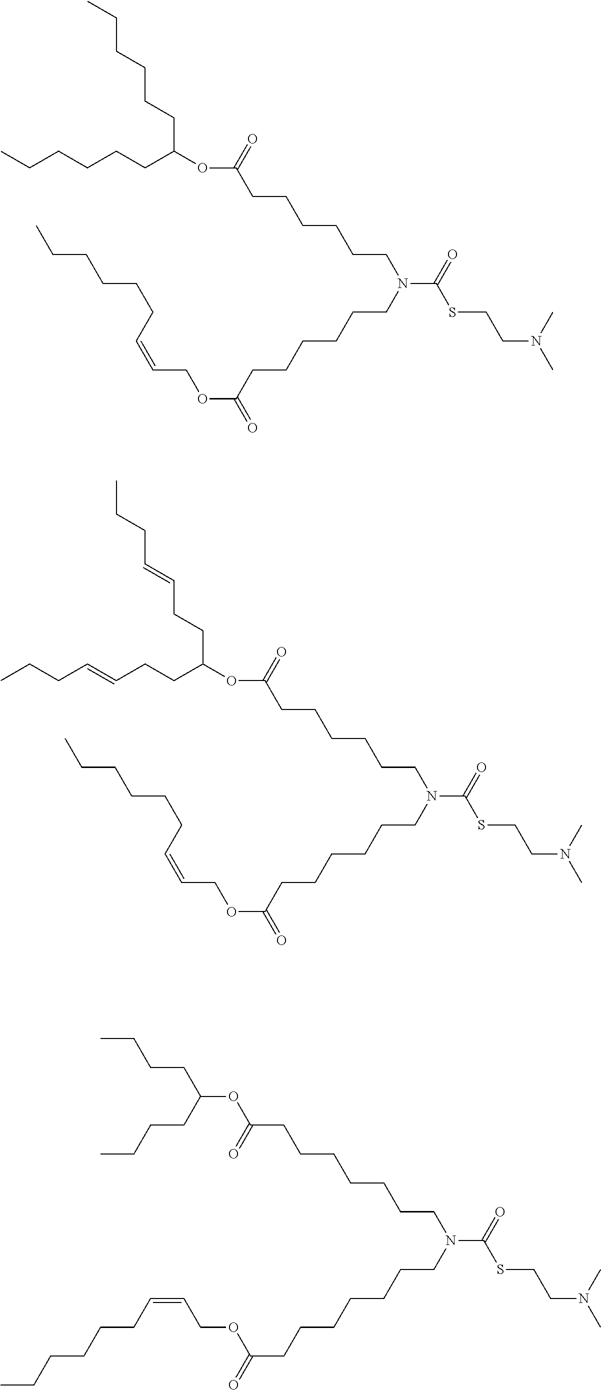

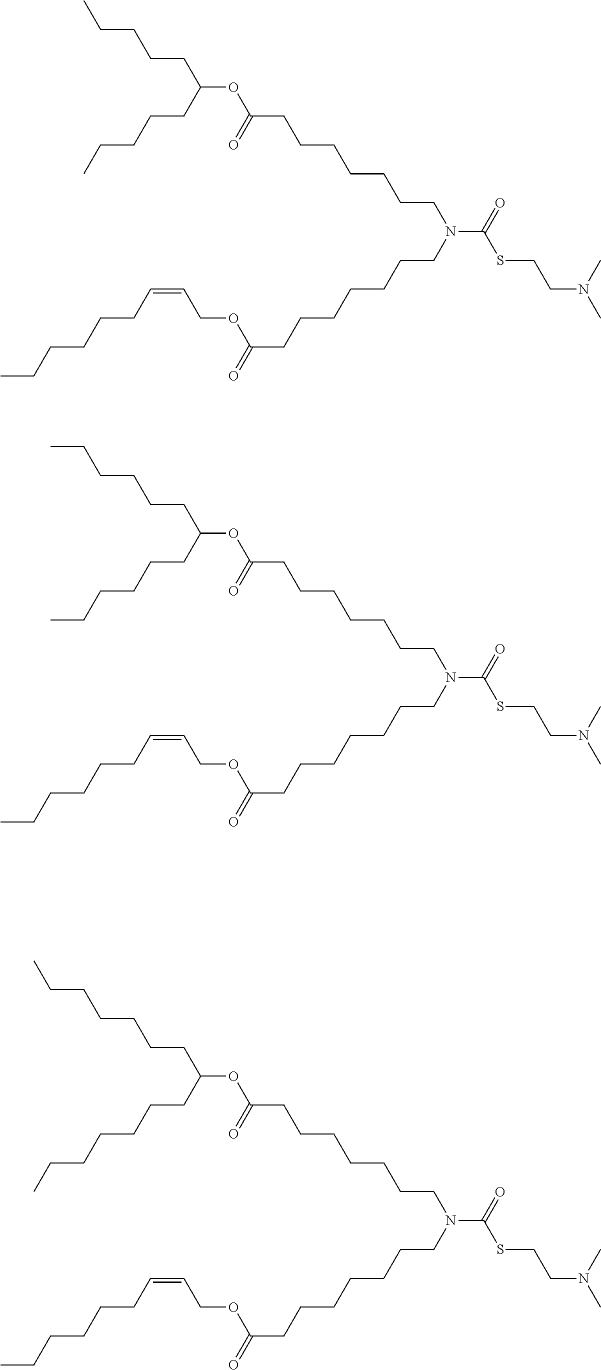

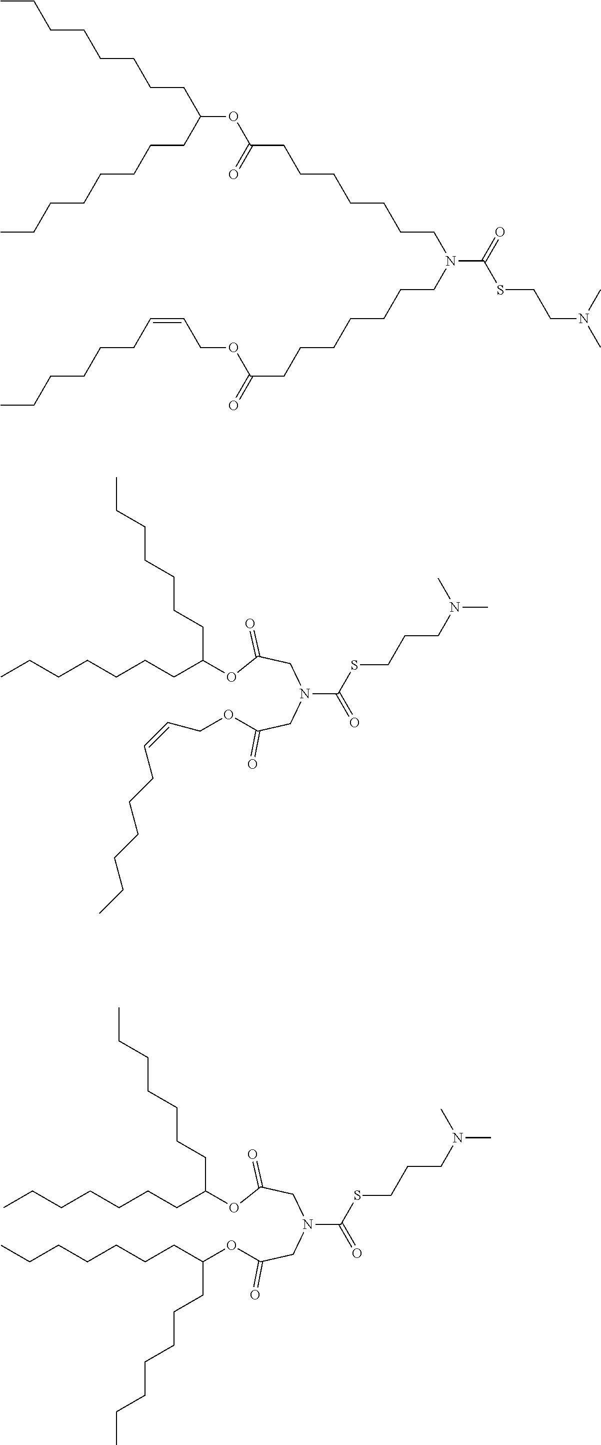

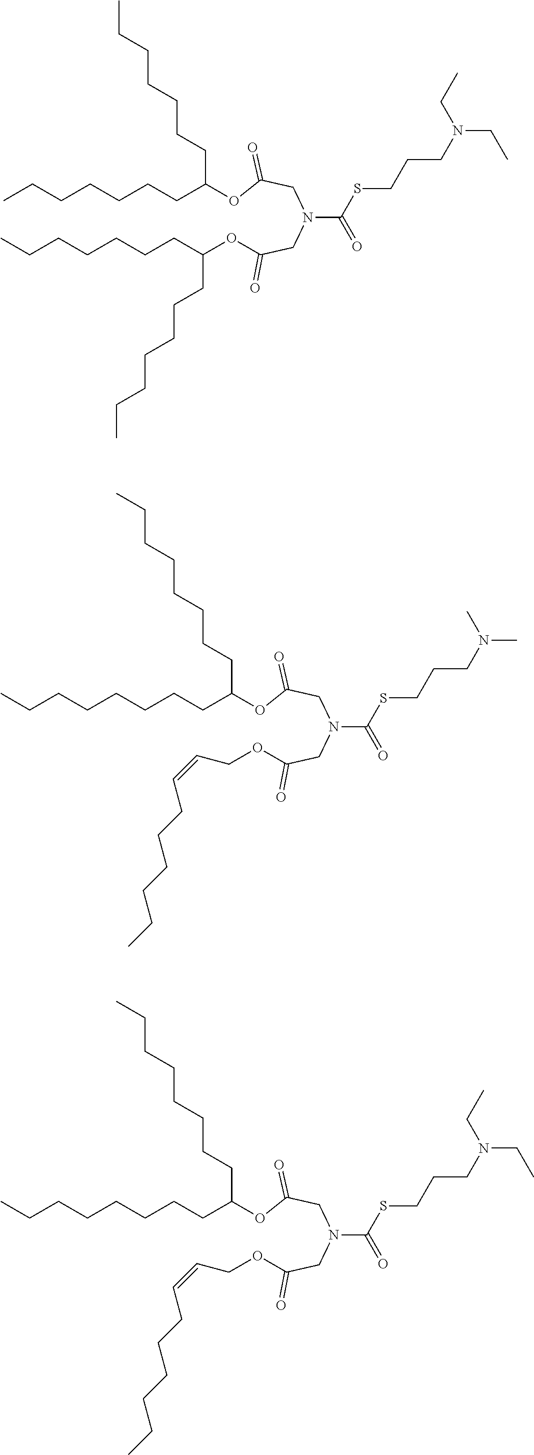

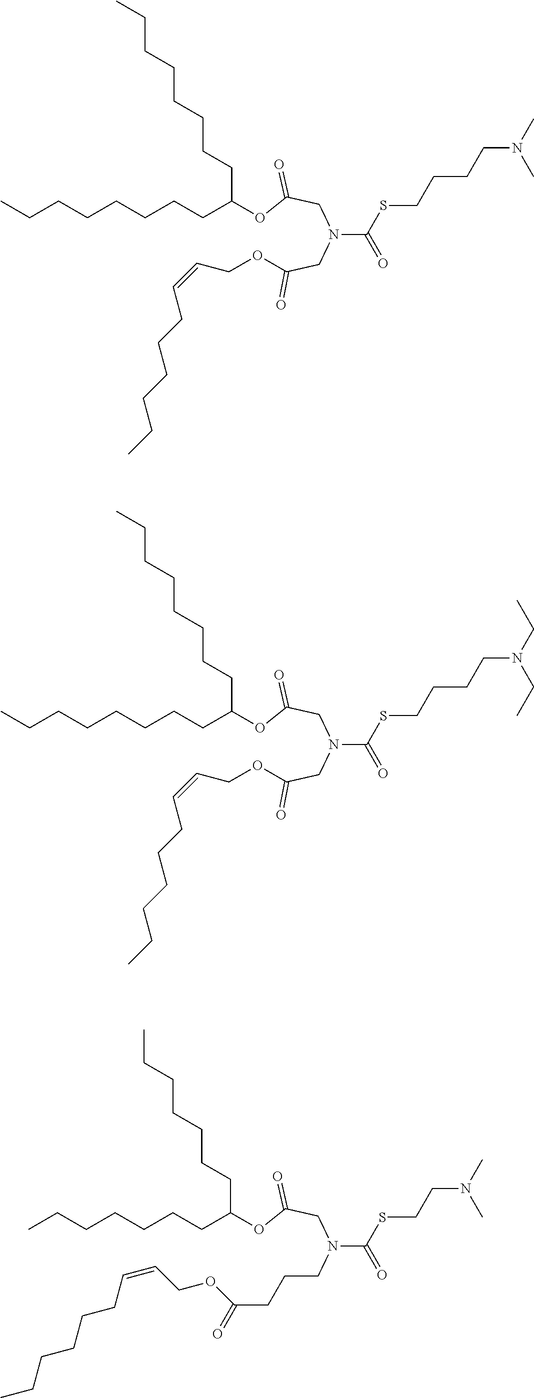

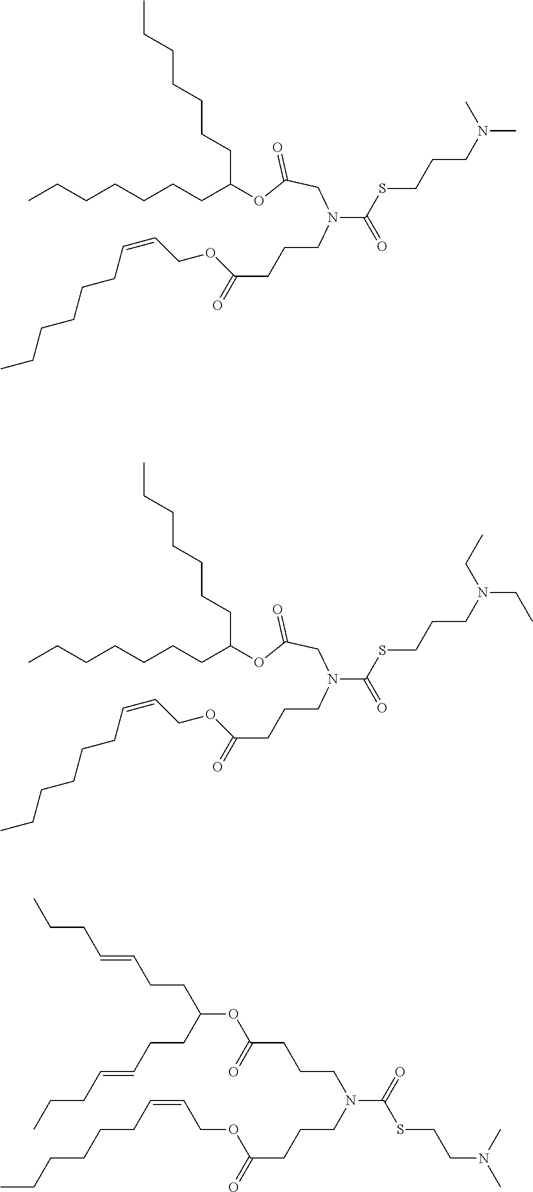

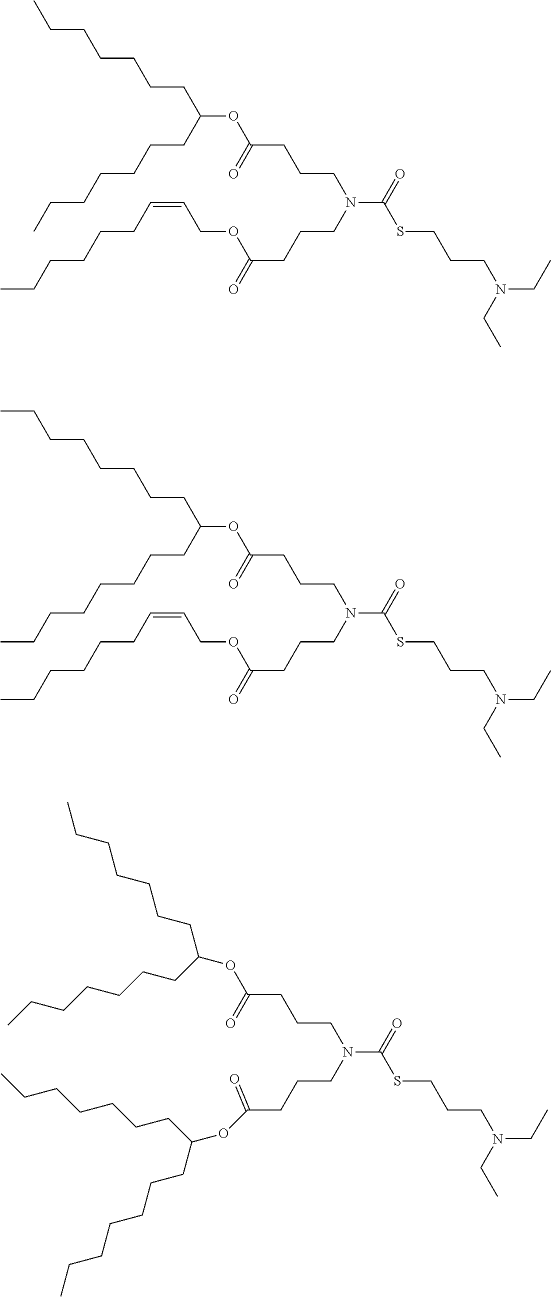

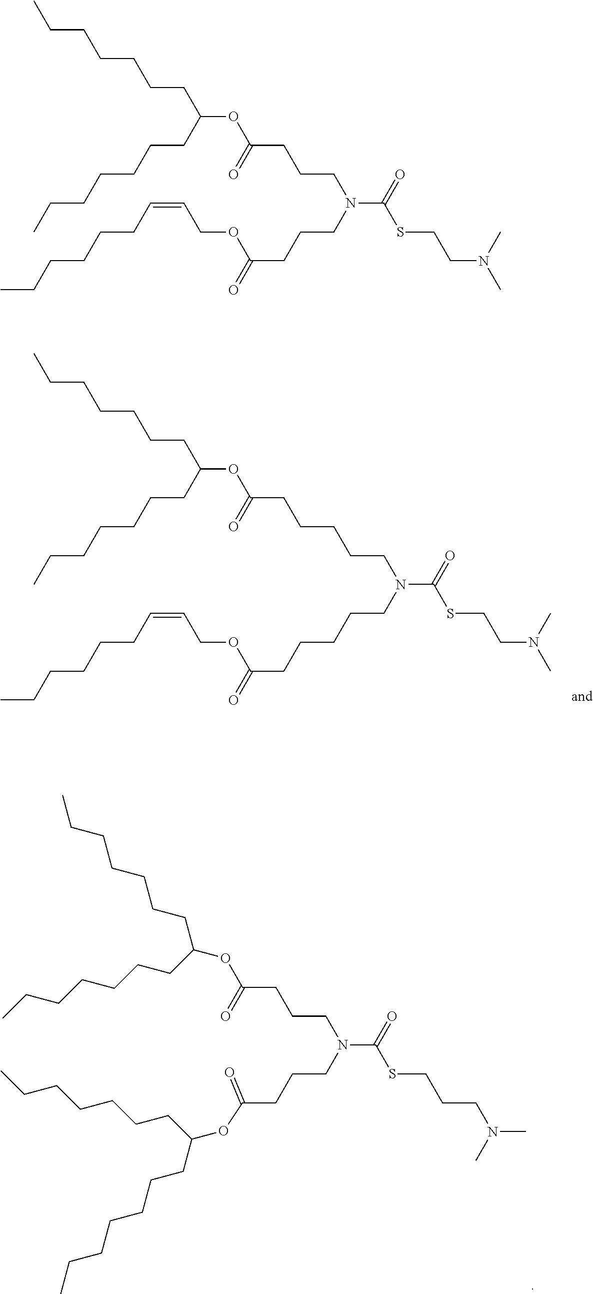

In some embodiments, the lipid formulation can compromise an ionizable cationic lipid selected from the group consisting of LIPID #1 to LIPID #5 as presented in Table 2:

TABLE 2

| LIPID # | STRUCTURE |
|---|---|
| 1 | 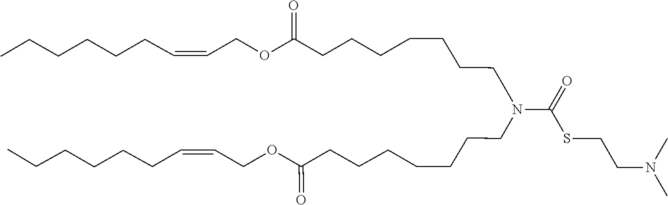 [SMILES: CCCCCC/C=C\COC(=O)CCCCCCCN(CCCCCCCC(=O)OC/C=C\CCCCCC)C(=O)SCCN(C)C] |
| 2 | 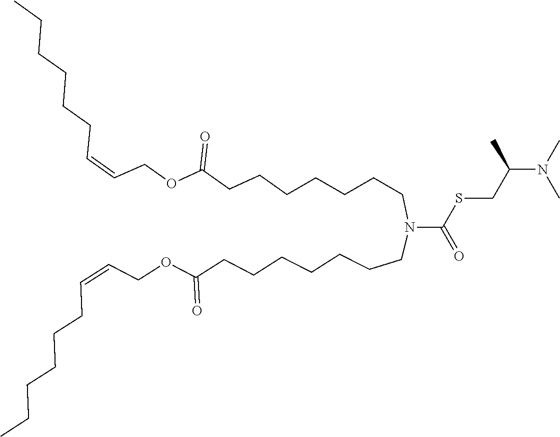 [SMILES: CCCCCC/C=C\COC(=O)CCCCCCCN(CCCCCCCC(=O)OC/C=C\CCCCCC)C(=O)SC[C@@H](C)N(C)C] |
| 3 | 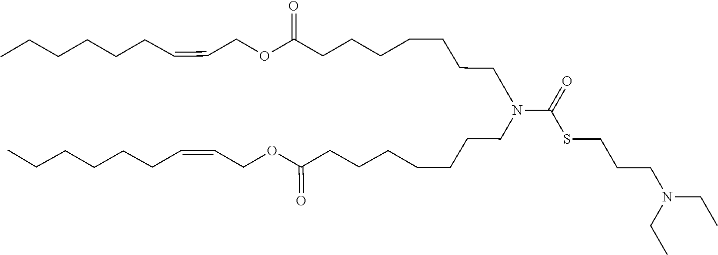 [SMILES: CCCCCC/C=C\COC(=O)CCCCCCCN(CCCCCCCC(=O)OC/C=C\CCCCCC)C(=O)SCCCN(CC)CC] |
| 4 | 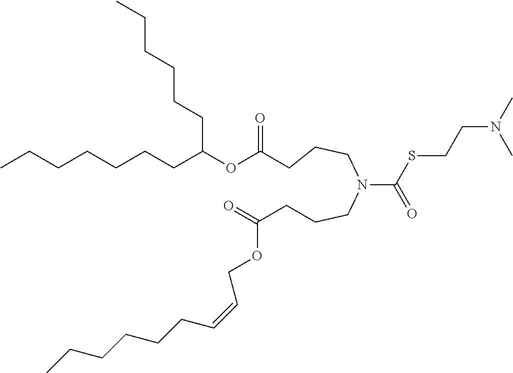 [SMILES: CCCCCC/C=C\COC(=O)CCCN(CCCC(=O)OC(CCCCCC)CCCCCCC)C(=O)SCCN(C)C] |
| 5 | 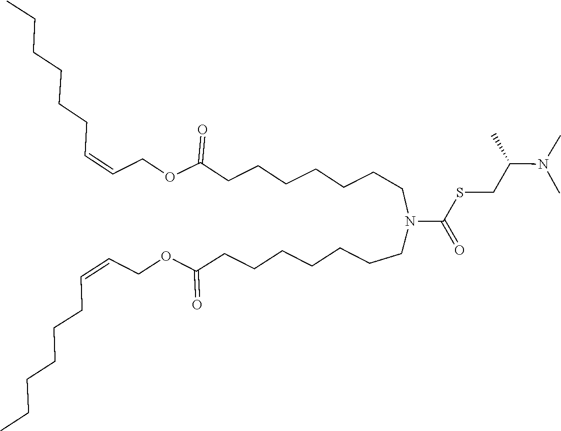 [SMILES: CCCCCC/C=C\COC(=O)CCCCCCCN(CCCCCCCC(=O)OC/C=C\CCCCCC)C(=O)SC[C@H](C)N(C)C] |

In some preferred embodiments, the lipid formulation comprises an ionizable cationic lipid having the structure

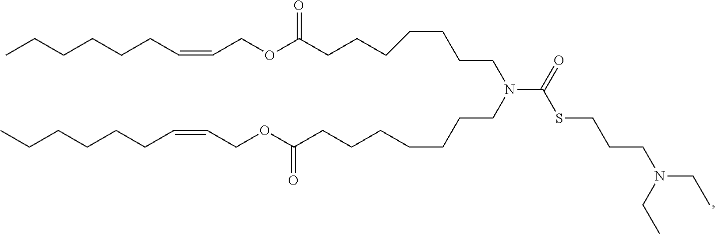 [SMILES: CCCCCC/C=C\COC(=O)CCCCCCCN(CCCCCCCC(=O)OC/C=C\CCCCCC)C(=O)SCCCN(CC)CC]

or a pharmaceutically acceptable salt thereof.

In some embodiments, any one or more lipids recited herein may be expressly excluded.

### Helper Lipids and Sterols

The mRNA-lipid formulations of the present disclosure can comprise a helper lipid, which can be referred to as a neutral lipid, a neutral helper lipid, non-cationic lipid, non-cationic helper lipid, anionic lipid, anionic helper lipid, or a zwitterionic lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoyl-sn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the helper lipid present in the lipid formulation comprises or consists of one or more phospholipids, e.g., a cholesterol-free lipid formulation. In yet other embodiments, the helper lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation.

In some embodiments, the total of helper lipid in the formulation comprises two or more helper lipids and the total amount of helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation. In some embodiments, the helper lipids are a combination of DSPC and DOTAP. In some embodiments, the helper lipids are a combination of DSPC and DOTMA.

The cholesterol or cholesterol derivative in the lipid formulation may comprise up to about 40 mol %, about 45 mol %, about 50 mol %, about 55 mol %, or about 60 mol % of the total lipid present in the lipid formulation. In some embodiments, the cholesterol or cholesterol derivative comprises about 15 mol % to about 45 mol %, about 20 mol % to about 40 mol %, about 30 mol % to about 40 mol %, or about 35 mol %, about 36 mol %, about 37 mol %, about 38 mol %, about 39 mol %, or about 40 mol % of the total lipid present in the lipid formulation.

The percentage of helper lipid present in the lipid formulation is a target amount, and the actual amount of helper lipid present in the formulation may vary, for example, by +5 mol %.

A lipid formulation containing a cationic lipid compound or ionizable cationic lipid compound may be on a molar basis about 20-40% cationic lipid compound, about 25-40% cholesterol, about 25-50% helper lipid, and about 0.5-5% of a polyethylene glycol (PEG) lipid, wherein the percent is of the total lipid present in the formulation. In some embodiments, the composition is about 22-30% cationic lipid compound, about 30-40% cholesterol, about 30-40% helper lipid, and about 0.5-3% of a PEG-lipid, wherein the percent is of the total lipid present in the formulation.

### Lipid Conjugates

The lipid formulations described herein may further comprise a lipid conjugate. The conjugated lipid is useful for preventing the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front. Pharmacol. 2015 Dec. 1; 6:286).

In a preferred embodiment, the lipid conjugate is a PEG-lipid. The inclusion of polyethylene glycol (PEG) in a lipid formulation as a coating or surface ligand, a technique referred to as PEGylation, helps protect nanoparticles from the immune system and their escape from RES uptake (Nanomedicine (Lond). 2011 June; 6(4):715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (J. Nanomaterials. 2011; 2011:12). It has been shown that increased PEGylation leads to a significant increase in the circulation half-life of lipid formulations (Annu. Rev. Biomed. Eng. 2011 Aug. 15; 13( ):507-30; J. Control Release. 2010 Aug. 3; 145(3):178-81).

Suitable examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH₂).

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In preferred embodiments, the PEG moiety has an average molecular weight of about 2,000 daltons or about 750 daltons. The average molecular weight may be any value or subvalue within the recited ranges, including endpoints.

In certain instances, the PEG monomers can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In a preferred embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, as well as combinations thereof (such as a linker containing both a carbamate linker moiety and an amido linker moiety). In a preferred embodiment, a carbamate linker is used to couple the PEG to the lipid.

In other embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some embodiments, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 to about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some embodiments, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

In some preferred embodiments, the PEG-lipid is PEG550-PE. In some preferred embodiments, the PEG-lipid is PEG750-PE. In some preferred embodiments, the PEG-lipid is PEG2000-DMG

The percentage of lipid conjugate (e.g., PEG-lipid) present in the lipid formulations of the disclosure is a target amount, and the actual amount of lipid conjugate present in the formulation may vary, for example, by +0.5 mol %. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of a CFTR mRNA-lipid formulation described herein, the mRNA-lipid formulation enters lung epithelial cells through receptor mediated endocytosis. Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For mRNA payloads, the cell's own internal translation processes will then translate the mRNA into the encoded protein. The encoded protein can further undergo post-translational processing, including transportation to a targeted organelle or location within the cell. In the case of a CFTR protein, the CFTR protein is translocated to the cellular membrane.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymmetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) a simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

CFTR mRNA Lipid Formulations

The present disclosure provides for lipid formulations comprising a mRNA encoding an enzyme having Cystic Fibrosis Transmembrane Conductance Regulator (CFTR) activity (CFTR mRNA). Following transfection of one or more target cells by the CFTR mRNA lipid formulations of the present disclosure, expression of the CFTR enzyme encoded by such mRNA will be stimulated and the capability of such target cells to express the CFTR enzyme is enhanced. The CFTR mRNA can be any suitable mRNA for expressing a CFTR enzyme in vivo.

In a first CFTR mRNA-lipid formulation, a CFTR mRNA-lipid formulation comprises a compound of Formula (I) and an mRNA encoding an enzyme having CFTR activity. In some embodiments the mRNA encodes a CFTR enzyme consisting of a sequence having 95% identity to SEQ ID NO: 93. In some embodiments, the mRNA encodes a CFTR enzyme consisting of SEQ ID NO: 93. In some embodiments the mRNA encodes a CFTR enzyme consisting of a sequence having 95% identity to SEQ ID NO: 99. In some embodiments, the mRNA encodes an CFTR enzyme consisting of SEQ ID NO: 99. The compound of Formula I can be selected based on desirable properties including its lipophilicity, potency, selectivity for a specific target cell, in vivo biodegradability, toxicity and immunogenicity profile, and the pKa of the ionizable/protonatable group on the compound of Formula I.

In some embodiments of the first CFTR mRNA-lipid formulation, X⁷ is S. In some embodiments, X⁵ is —C(O)O—, whereby —C(O)O—R⁶ is formed and X⁶ is —C(O)O— whereby —C(O)O—R⁵ is formed. In some embodiments, R⁷ and R⁸ are each independently selected from the group consisting of methyl, ethyl and isopropyl. In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl. In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkane. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C3 alkyl. In some embodiments, R⁵ consists of —CH((CH₂)ₚCH₃)((CH₂)ₚ₋₁CH₃), wherein p is 7 or 8. In some embodiments, R⁴ is ethylene or propylene. In some embodiments, R⁴ is n-propylene or isobutylene. In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments of the first CFTR mRNA-lipid formulation, X⁷ is S, X⁵ is —C(O)O—, whereby —C(O)O—R⁶ is formed and X⁶ is —C(O)O—, whereby —C(O)O—R⁵ is formed, L⁵ and L⁶ are each independently a linear C₃-C₇ alkyl L⁷ is absent, R⁵ is —CH((CH₂)ₚCH₃)₂, and R⁶ is C₇-C₁₂ alkenyl. In some further embodiments, p is 6 and R⁶ is C₉ alkenyl.

Any mRNA encoding an enzyme having CFTR activity is suitable for inclusion in the first CFTR mRNA-lipid formulation of the present disclosure. In some embodiments, a suitable mRNA is a wild-type human CFTR mRNA of sequence SEQ ID NO: 93. Preferably, the CFTR mRNA has low immunogenicity, high in vivo stability, and high translation efficiency. In some embodiments, the CFTR mRNA is expressible in human lung epithelial cells. In some embodiments, the CFTR mRNA has a coding region that is codon-optimized. In some embodiments, the CFTR mRNA comprises modified uridine nucleotides. In some embodiments, the modified uridine nucleotides are N¹-methylpseudouridine or 5-methoxyuridine. In some embodiments, the modified uridine nucleotides are 5-methoxyuridine. In some embodiments, the CFTR mRNA can be any of the CFTR mRNA constructs described herein.

In some embodiments of the first CFTR mRNA-lipid formulation, the mRNA comprises an open reading frame (ORF or coding region) selected from a sequence comprising SEQ ID NOs: 100-105. In some embodiments, the mRNA comprises an ORF having a sequence of SEQ ID NO: 100. In some embodiments, the mRNA comprises an ORF having a sequence of SEQ ID NO: 101. In some embodiments, the mRNA comprises an ORF having a sequence of SEQ ID NO: 102. In some embodiments, the mRNA comprises an ORF having a sequence of SEQ ID NO: 103. In some embodiments, the mRNA comprises an ORF having a sequence of SEQ ID NO: 104. In some embodiments, the mRNA comprises an ORF having a sequence of SEQ ID NO: 105. In some embodiments, the mRNA comprises a sequence having about 85% identity to a sequence selected from SEQ ID NOs: 49, 53, 66, 68, 69, and 72. In some embodiments, the mRNA comprises a sequence having about 90% identity to a sequence selected from SEQ ID NOs: 49, 53, 66, 68, 69, and 72. In some embodiments, the mRNA comprises a sequence having about 95% identity to a sequence selected from SEQ ID NOs: 49, 53, 66, 68, 69, and 72. In some embodiments, the mRNA comprises a sequence having about 96% identity to a sequence selected from SEQ ID NOs: 49, 53, 66, 68, 69, and 72. In some embodiments, the mRNA comprises a sequence having about 97% identity to a sequence selected from SEQ ID NOs: 49, 53, 66, 68, 69, and 72. In some embodiments, the mRNA comprises a sequence having about 98% identity to a sequence selected from SEQ ID NOs: 49, 53, 66, 68, 69, and 72. In some embodiments, the mRNA comprises a sequence having about 99% identity to a sequence selected from SEQ ID NOs: 49, 53, 66, 68, 69, and 72. In some embodiments, the mRNA comprises a sequence having about 99.5% identity to a sequence selected from SEQ ID NOs: 49, 53, 66, 68, 69, and 72. In some embodiments, the mRNA comprises a sequence selected from SEQ ID NOS: 49, 53, 66, 68, 69, and 72. In some embodiments, the mRNA comprises a sequence having SEQ ID NO: 49. In some embodiments, the mRNA comprises a sequence having SEQ ID NO: 53. In some embodiments, the mRNA comprises a sequence having SEQ ID NO: 66. In some embodiments, the mRNA comprises a sequence having SEQ ID NO: 68. In some embodiments, the mRNA comprises a sequence having SEQ ID NO: 69. In some embodiments, the mRNA comprises a sequence having SEQ ID NO: 72.

In any of the embodiments of the first CFTR mRNA-lipid formulation, the CFTR mRNA-lipid formulation comprises lipid nanoparticles. In some embodiments, the lipid nanoparticles completely encapsulate the CFTR mRNA.

In some embodiments, the lipid nanoparticles have an average particle size of less than about 100 nm. In some embodiments, the lipid nanoparticles have an average particles size of about 55 to about 85 nm. In some embodiments, the lipid nanoparticles encapsulate at least about 50% of the mRNA. In some embodiments, the lipid nanoparticles encapsulate at least about 85% of the mRNA. In some embodiments, the lipid nanoparticles have greater than about 90% encapsulation efficiency. In some embodiments, the lipid nanoparticles have greater than about 95% encapsulation efficiency.

In a second CFTR mRNA-lipid formulation, a CFTR mRNA-lipid formulation comprises the ionizable cationic lipid

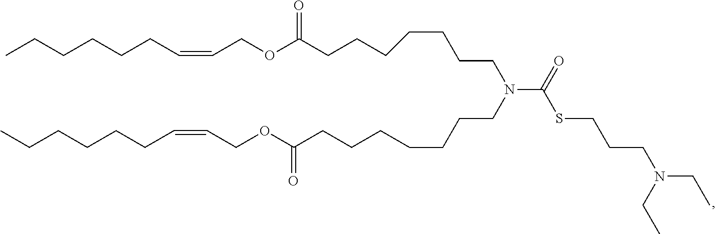 [SMILES: CCCCCC/C=C\COC(=O)CCCCCCCN(CCCCCCCC(=O)OC/C=C\CCCCCC)C(=O)SCCCN(CC)CC]

or a pharmaceutically acceptable salt thereof and an mRNA encoding an enzyme having CFTR activity.

Any mRNA encoding an enzyme having CFTR activity is suitable for inclusion in the second CFTR mRNA-lipid formulation of the present disclosure. In some embodiments, a suitable mRNA is a wild-type human CFTR mRNA encoding a protein of SEQ ID NO: 93. In some embodiments the mRNA encodes a CFTR enzyme consisting of a sequence having 95% identity to SEQ ID NO: 93. In some embodiments, the mRNA encodes a CFTR enzyme consisting of SEQ ID NO: 93. In some embodiments the mRNA encodes a CFTR enzyme consisting of a sequence having 95% identity to SEQ ID NO: 99. In some embodiments, the mRNA encodes a CFTR enzyme consisting of SEQ ID NO: 99. Preferably, the CFTR mRNA has low immunogenicity, high in vivo stability, and high translation efficiency. In some embodiments, the CFTR mRNA is expressible in human lung epithelial cells. In some embodiments, the CFTR mRNA has a coding region that is codon-optimized. In some embodiments, the CFTR mRNA comprises modified uridine nucleotides. In some embodiments, the modified uridine nucleotides are N¹-methylpseudouridine or 5-methoxyuridine. In some embodiments, the modified uridine nucleotides are 5-methoxyuridine. In some embodiments, the modified uridine nucleotides are N¹-methylpseudouridine. In some embodiments, the CFTR mRNA can be any of the CFTR mRNA constructs described herein.

In some embodiments of the second CFTR mRNA-lipid formulation, the mRNA comprises an open reading frame (ORF or coding region) selected from a sequence comprising SEQ ID NOs: 100-105. In some embodiments, the mRNA comprises an ORF having a sequence of SEQ ID NO: 100. In some embodiments, the mRNA comprises an ORF having a sequence of SEQ ID NO: 101. In some embodiments, the mRNA comprises an ORF having a sequence of SEQ ID NO: 102. In some embodiments, the mRNA comprises an ORF having a sequence of SEQ ID NO: 103. In some embodiments, the mRNA comprises an ORF having a sequence of SEQ ID NO: 104. In some embodiments, the mRNA comprises an ORF having a sequence of SEQ ID NO: 105. In some embodiments, the mRNA comprises a sequence having about 85% identity to a sequence selected from SEQ ID NOs: 49, 53, 66, 68, 69, and 72. In some embodiments, the mRNA comprises a sequence having about 90% identity to a sequence selected from SEQ ID NOs: 49, 53, 66, 68, 69, and 72. In some embodiments, the mRNA comprises a sequence having about 95% identity to a sequence selected from SEQ ID NOs: 49, 53, 66, 68, 69, and 72. In some embodiments, the mRNA comprises a sequence having about 96% identity to a sequence selected from SEQ ID NOs: 49, 53, 66, 68, 69, and 72. In some embodiments, the mRNA comprises a sequence having about 97% identity to a sequence selected from SEQ ID NOs: 49, 53, 66, 68, 69, and 72. In some embodiments, the mRNA comprises a sequence having about 98% identity to a sequence selected from SEQ ID NOs: 49, 53, 66, 68, 69, and 72. In some embodiments, the mRNA comprises a sequence having about 99% identity to a sequence selected from SEQ ID NOs: 49, 53, 66, 68, 69, and 72. In some embodiments, the mRNA comprises a sequence having about 99.5% identity to a sequence selected from SEQ ID NOs: 49, 53, 66, 68, 69, and 72. In some embodiments, the mRNA comprises a sequence selected from SEQ ID NOS: 49, 53, 66, 68, 69, and 72. In some embodiments, the mRNA comprises a sequence having SEQ ID NO: 49. In some embodiments, the mRNA comprises a sequence having SEQ ID NO: 53. In some embodiments, the mRNA comprises a sequence having SEQ ID NO: 66. In some embodiments, the mRNA comprises a sequence having SEQ ID NO: 68. In some embodiments, the mRNA comprises a sequence having SEQ ID NO: 69. In some embodiments, the mRNA comprises a sequence having SEQ ID NO: 72.

In any of the embodiments of the second CFTR mRNA-lipid formulation, the CFTR mRNA-lipid formulation comprises lipid nanoparticles. In some embodiments, the lipid nanoparticles completely encapsulate the CFTR mRNA.

In some embodiments, the lipid nanoparticles have an average particle size of less than about 100 nm. In some embodiments, the lipid nanoparticles have an average particles size of about 55 nm to about 85 nm. In some embodiments, the lipid nanoparticles encapsulate at least about 50% of the mRNA. In some embodiments, the lipid nanoparticles encapsulate at least about 85% of the mRNA. In some embodiments, the lipid nanoparticles have greater than about 90% encapsulation efficiency.

In some embodiments, either the first or second CFTR mRNA-lipid formulation further comprises a helper lipid. In some embodiments, the helper lipid is selected from the group consisting of neutral and anionic lipids. In some embodiments, the helper lipid is selected from the group consisting of dipalmitoyl phosphatidylcholine (DPPC), phosphatidylcholine (PC), dioleoylphosphatidyl ethanolamine (DOPE), dimyristoylphosphatidyl choline (DMPC), distearoylphosphatidyl choline, and dimyristoylphosphatidyl glycerol (DMPG). In some embodiments, the non-cationic lipid is distearoylphosphatidylcholine (DSPC).

In some embodiments, either the first or second CFTR mRNA-lipid formulation further comprises cholesterol.

In some embodiments, either the first or second CFTR mRNA-lipid formulation further comprises a polyethylene glycol (PEG)-lipid conjugate. In some embodiments, the PEG-lipid conjugate is PEG-DMG. In some embodiments, the PEG-DMG is PEG2000-DMG.

In some embodiments, the lipid portion (meaning the total amount of lipids in the formulation) of either the first or second CFTR mRNA-lipid formulation comprises about 48 mol % to about 66 mol % of the cationic lipid, about 2 mol % to about 12 mol % DSPC, about 25 mol % to about 42 mol % cholesterol, and about 0.5 mol % to about 3 mol % PEG2000-DMG.

In some embodiments, the lipid portion of either the first or second CFTR mRNA-lipid formulation comprises about 55 mol % to about 61 mol % of the cationic lipid, about 5 mol % to about 9 mol % DSPC, about 29 mol % to about 38 mol % cholesterol, and about 1 mol % to about 2 mol % PEG2000-DMG.

In some embodiments, the lipid portion of either the first or second CFTR mRNA-lipid formulation comprises about 56 mol % to about 60 mol % of the cationic lipid, about 6 mol % to about 8 mol % DSPC, about 31 mol % to about 34 mol % cholesterol, and about 1.25 mol % to about 1.75 mol % PEG2000-DMG.

In some embodiments, either the first or second CFTR mRNA-lipid formulation has a total lipid:mRNA weight ratio of about 50:1 to about 10:1. In some embodiments, either the first or second CFTR mRNA-lipid formulation has a total lipid:mRNA weight ratio of about 40:1 to about 20:1. In some embodiments, either the first or second CFTR mRNA-lipid formulation has a total lipid:mRNA weight ratio of about 35:1 to about 25:1. In some embodiments, either the first or second CFTR mRNA-lipid formulation has a total lipid:mRNA weight ratio of about 28:1 to about 32:1. In some embodiments, either the first or second CFTR mRNA-lipid formulation has a total lipid:mRNA weight ratio of about 29:1 to about 31:1.

Pharmaceutical Compositions Comprising CFTR mRNA and Lipid Formulations Containing Cationic Lipid ATX-012

The present disclosure provides for pharmaceutical compositions comprising (a) a lipid formulation comprising an ionizable cationic lipid, wherein the ionizable cationic lipid is ATX-012; and (b) a messenger RNA (mRNA) encoding a peptide having cystic fibrosis transmembrane conductance regulator (CFTR) activity; wherein the lipid formulation encapsulates the mRNA.

In some further embodiments, the lipid formulation of the pharmaceutical composition comprises a first helper lipid which is DOTAP, a second helper lipid, a PEG-lipid conjugate and cholesterol. In some embodiments, the second helper lipid is DSPC. In some embodiments, the PEG-lipid conjugate is PEG-DMG.

In some further embodiments, the mRNA of the pharmaceutical composition has a sequence selected from the group consisting of SEQ ID NOs: 49, 53, 66, 68, 69 and 72.

In some further embodiments, the peptide having cystic fibrosis transmembrane conductance regulator (CFTR) activity has a sequence at least about 90% identical to a sequence of SEQ ID NO: 99. In some further embodiments, the peptide having cystic fibrosis transmembrane conductance regulator (CFTR) activity has a sequence at least about 95% identical to a sequence of SEQ ID NO: 99. In some embodiments, the peptide having CFTR activity has a sequence at least about 98% identical to a sequence of SEQ ID NO: 99. In some embodiments, the peptide having CFTR activity has a sequence at least about 99% identical to a sequence of SEQ ID NO: 99. In some embodiments, the peptide having CFTR activity has a sequence of SEQ ID NO: 99.

In some particular embodiments, there is provided a pharmaceutical composition comprising:

(a) a lipid formulation comprising:
i. about 20 mol % to about 30 mol % of an ionizable cationic lipid having the structure of ATX-012:

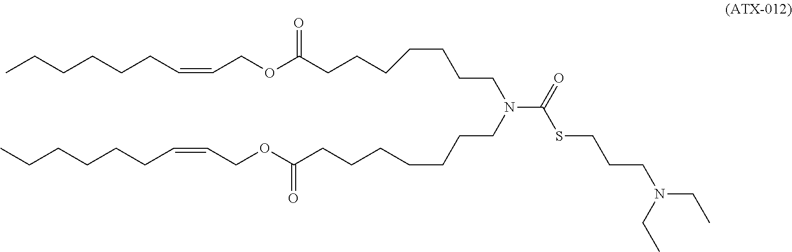 [SMILES: CCCCCC/C=C\COC(=O)CCCCCCCN(CCCCCCCC(=O)OC/C=C\CCCCCC)C(=O)SCCCN(CC)CC]

vi. about 20 mol % to about 30 mol % 1,2-dioleoyl-3-trimethylammonium-propane (DOTAP);
vii. about 7 mol % to about 13 mol % of a helper lipid;
viii. about 33 mol % to about 44 mol % cholesterol; and
ix. about 0.5 mol % to about 3.0 mol % of a PEG-lipid conjugate; and

(b) a messenger RNA (mRNA) encoding a peptide having cystic fibrosis transmembrane conductance regulator (CFTR) activity;

wherein the lipid formulation encapsulates the mRNA.

In some embodiments, the lipid formulation (a) is selected from the group consisting of a lipoplex, a liposome, a lipid nanoparticle, a polymer-based carrier, an exosome, a lamellar body, a micelle and an emulsion.

In some embodiments, the lipid formulation (a) is a liposome. In some embodiments, the liposome is selected from the group consisting of a cationic liposome, a nanoliposome, a proteoliposome, a unilamellar liposome, a multilamellar liposome, a ceramide-containing nanoliposome and a multivesicular liposome.

In some embodiments, the lipid formulation (a) is a lipid nanoparticle. In some embodiments, the lipid nanoparticle has a size of less than about 200 nm. In some embodiments, the lipid nanoparticle has a size of less than about 150 nm. In some embodiments, the lipid nanoparticle has a size of less than about 100 nm. In some embodiments, the lipid nanoparticle has a size of about 55 nm to about 90 nm. The values and ranges recited herein include any subvalue or subrange therebetween.

In some embodiments, the helper lipid of lipid formulation (a) is a phospholipid. In some embodiments, the helper lipid of lipid formulation (a) is selected from the group consisting of dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC) and phosphatidylcholine (PC). In some embodiments, the helper lipid is distearoylphosphatidylcholine (DSPC). In some embodiments, the lipid formulation (a) comprises about 8 mol % to about 12 mol % of the helper lipid. In some embodiments, the lipid formulation (a) comprises about 9 mol % to about 11 mol % of the helper lipid. In some embodiments, the lipid formulation (a) comprises about 10 mol % of the helper lipid.

In some embodiments, the PEG-lipid conjugate of lipid formulation (a) is PEG-DMG. In some embodiments, the PEG-DMG is PEG2000-DMG. In some embodiments, the lipid formulation (a) comprises about 0.75 mol % to about 2.5 mol % of the PEG-lipid conjugate. In some embodiments, the lipid formulation (a) comprises about 1.0 mol % to about 2.0 mol % of the PEG-lipid conjugate. In some embodiments, the lipid formulation (a) comprises about 1.25 mol % to about 1.75 mol % of the PEG-lipid conjugate. In some embodiments, the lipid formulation (a) comprises about 1.5 mol % of the PEG-lipid conjugate.

In some embodiments, the lipid formulation (a) comprises about 22 mol % to about 28 mol % of the ionizable cationic lipid ATX-012. In some embodiments, the lipid formulation (a) comprises about 23 mol % to about 27 mol % of the ionizable cationic lipid ATX-012. In some embodiments, the lipid formulation (a) comprises about 24 mol % to about 26 mol % of the ionizable cationic lipid ATX-012. In some embodiments, the lipid formulation (a) comprises about 25 mol % of the ionizable cationic lipid ATX-012.

In some embodiments, the lipid formulation (a) comprises about 22 mol % to about 28 mol % DOTAP. In some embodiments, the lipid formulation (a) comprises about 23 mol % to about 27 mol % DOTAP. In some embodiments, the lipid formulation (a) comprises about 24 mol % to about 26 mol % DOTAP. In some embodiments, the lipid formulation (a) comprises about 25 mol % DOTAP.

In some embodiments, the lipid formulation (a) comprises about 35 mol % to about 41 mol % cholesterol. In some embodiments, the lipid formulation (a) comprises about 36 mol % to about 40 mol % cholesterol.

In some embodiments, the pharmaceutical composition has a total lipid:mRNA weight ratio of about 5:1 to about 25:1. In some embodiments, the composition has a total lipid:mRNA weight ratio of about 10:1 to about 20:1. In some embodiments, the composition has a total lipid:mRNA weight ratio of about 12:1 to about 18:1. In some embodiments, the composition has a total lipid:mRNA weight ratio of about 14:1 to about 17:1. In some embodiments, the composition has a total lipid:mRNA weight ratio of about 14:1 to about 16:1. In some embodiments, the composition has a total lipid:mRNA weight ratio of about 15:1.

In some embodiments, the pharmaceutical composition comprises the mRNA encoding the peptide having CFTR activity, wherein the peptide has a sequence at least about 85% identical to a sequence of SEQ ID NO: 99. In some embodiments, the peptide having CFTR activity has sequence at least about 90% identical to a sequence of SEQ ID NO: 99. In some embodiments, the peptide having CFTR activity has a sequence at least about 95% identical to a sequence of SEQ ID NO: 99. In some embodiments, the peptide having CFTR activity has a sequence at least about 98% identical to a sequence of SEQ ID NO: 99. In some embodiments, the peptide having CFTR activity has a sequence at least about 99% identical to a sequence of SEQ ID NO: 99. In some embodiments, the peptide having CFTR activity has a sequence of SEQ ID NO: 99.

In some embodiments, the mRNA of the pharmaceutical composition has a sequence selected from the group consisting of SEQ ID NOs: 49, 53, 66, 68, 69 and 72. In some embodiments, the mRNA comprises SEQ ID NO: 49. In some embodiments, the mRNA comprises SEQ ID NO: 53. In some embodiments, the mRNA comprises SEQ ID NO: 66. In some embodiments, the mRNA comprises SEQ ID NO: 68. In some embodiments, the mRNA comprises SEQ ID NO: 69. In some embodiments, the mRNA comprises SEQ ID NO: 72.

In some embodiments, the mRNA of the pharmaceutical composition comprises a 3′ poly-A tail. In some embodiments, 3′ poly-A tail consists of about 50 to about 120 adenosine monomers.

In some embodiments, the mRNA of the pharmaceutical composition comprises a 5′ cap. In some embodiments, the 5′ cap is m⁷GpppAmpG having the structure of Formula (Cap V):

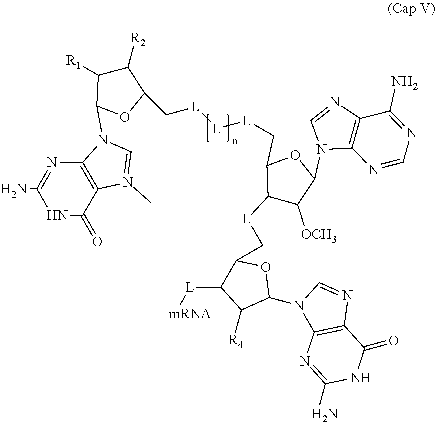

wherein R¹, R², and R⁴ are each OH, n is 1, each L is a phosphate linked by diester bonds, and mRNA is the mRNA of the composition.

In some embodiments, the mRNA of the pharmaceutical composition comprises one or more chemically-modified nucleotides each independently selected from the group consisting of 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine and 6-O-methylguanosine. In some embodiments, the one or more chemically modified nucleotides is an N¹-methylpseudouridine.

In some embodiments, the pharmaceutical composition comprises a buffer. In some embodiments, the buffer is HEPES or TRIS buffer. In some embodiments, the HEPES or TRIS buffer pH is about 7.0 to about 8.5. In some embodiments, the HEPES or TRIS buffer pH is about 7.4 to about 8.2. In some embodiments, the HEPES or TRIS buffer concentration is about 20 mM to about 80 mM. In some embodiments, the buffer is HEPES at a concentration of about 35 mM to about 70 mM. In some embodiments, the buffer is HEPES at a concentration of about 40 mM to about 60 mM. In some embodiments, the buffer is HEPES at a concentration of about 45 mM to about 55 mM. In some embodiments, the buffer is TRIS at a concentration of about 20 mM to about 50 mM. In some embodiments, the buffer is TRIS at a concentration of about 25 mM to about 40 mM. In some embodiments, the buffer is TRIS at a concentration of about 25 mM to about 35 mM.

In some embodiments, the pharmaceutical composition comprises sodium chloride (NaCl). In some embodiments, the NaCl concentration is about 10 mM to about 100 mM of NaCl. In some embodiments, the NaCl concentration is about 20 mM to about 90 mM of NaCl. In some embodiments, the NaCl concentration is about 30 mM to about 80 mM of NaCl. In some embodiments, the NaCl concentration is about 35 mM to about 70 mM of NaCl. In some embodiments, the NaCl concentration is about 40 mM to about 60 mM of NaCl. In some embodiments, the NaCl concentration is about 45 mM to about 55 mM of NaCl.

In some embodiments, the pharmaceutical composition comprises one or more cryoprotectants. In some embodiments, the one or more cryoprotectants is selected from the group consisting of sucrose, glycerol, and a combination of sucrose and glycerol. In some embodiments, the cryoprotectant is sucrose. In some embodiments, the cryoprotectant is glycerol. In some embodiments, the cryoprotectant is a combination of sucrose and glycerol. In some embodiments, the pharmaceutical composition comprises a combination of sucrose at a concentration of about 5% w/v to about 18% w/v and glycerol at a concentration of about 1% w/v to about 9% w/v. In some embodiments, the pharmaceutical composition comprises a combination of sucrose at a concentration of about 6% w/v to about 16% w/v and glycerol at a concentration of about 1.5% w/v to about 7% w/v. In some embodiments, the pharmaceutical composition comprises a combination of sucrose at a concentration of about 7% w/v to about 14% w/v and glycerol at a concentration of about 1.75% w/v to about 6% w/v. In some embodiments, the pharmaceutical composition comprises a combination of sucrose at a concentration of about 7% w/v to about 12% w/v and glycerol at a concentration of about 1% w/v to about 6% w/v. In some embodiments, the pharmaceutical composition comprises a combination of sucrose at a concentration of about 8% w/v to about 11% w/v and glycerol at a concentration of about 3% w/v to about 6% w/v.

In some more particular embodiments, the pharmaceutical composition comprises:
the lipid formulation (a), wherein the helper lipid is distearoylphosphatidylcholine (DSPC), and the PEG-lipid conjugate is PEG2000-DMG; and
the mRNA (b), wherein the mRNA comprises SEQ ID NO: 53.

In some further embodiments, the foregoing lipid formulation is a lipid nanoparticle having a size of less than about 100 nm.

In some further embodiments, the total lipids:mRNA weight ratio is within a range of about 10:1 to about 20:1; and the peptide having CFTR activity has a sequence at least about 95% identical to a sequence of SEQ ID NO: 99. In some embodiments, the peptide having CFTR activity has a sequence of SEQ ID NO: 99.

In some further embodiments, the pharmaceutical composition further comprises a HEPES or TRIS buffer, wherein the buffer pH is within a range of about 7.0 to about 8.5.

In some further embodiments, the pharmaceutical composition comprises NaCl. In some embodiments, the NaCl concentration in the pharmaceutical composition is about 10 mM to about 100 mM.

In yet some further embodiments, the pharmaceutical composition comprises one or more cryoprotectants. In some embodiments, the one or more cryoprotectants is selected from the group consisting of sucrose, glycerol, and a combination of sucrose and glycerol. In some embodiments, the one or more cryoprotectants is a combination of sucrose and glycerol.

In some more particular embodiments, the lipid formulation (a) comprises:
about 23 mol % to about 27 mol % of the ionizable cationic lipid ATX-012;
about 22 mol % to about 28 mol % DOTAP;
about 35 mol % to about 41 mol % cholesterol; and
about 0.75 mol % to about 2.5 mol % of PEG-DMG.

In some further embodiments, the lipid nanoparticle has a size of within a range of about 50 nm to about 90 nm.

The present disclosure also provides for use of a pharmaceutical composition of any one of the foregoing embodiments for manufacturing a medicament for ameliorating, preventing, delaying onset, or treating a disease or disorder associated with reduced activity of Cystic Fibrosis Transmembrane Conductance Regulator (CFTR) in a subject need thereof. In some embodiments, the disease is Cystic Fibrosis having a Cystic Fibrosis mutation. In some embodiments, the Cystic Fibrosis mutation is selected from the group consisting of Class 1A, Class 1B, Class 3, Class 4, Class 5 and Class 6. In some embodiments, the Cystic Fibrosis mutation is Class 1A. In some embodiments, the Cystic Fibrosis mutation is Class 1B. In some embodiments, the Cystic Fibrosis mutation is Class 3. In some embodiments, the Cystic Fibrosis mutation is Class 4. In some embodiments, the Cystic Fibrosis mutation is Class 5. In some embodiments, the Cystic Fibrosis mutation is Class 6.

The present disclosure also provides for a method for ameliorating, preventing, delaying onset, or treating a disease or disorder associated with reduced activity of Cystic Fibrosis Transmembrane Conductance Regulator (CFTR) in a subject in need thereof, the method comprising administering to the subject a pharmaceutical composition of any one of the foregoing embodiments. In some embodiments, the disease or disorder is Cystic Fibrosis. In some embodiments, the administration is intravenous, subcutaneous, pulmonary, intramuscular, intraperitoneal, dermal, oral, nasal or inhalation. In some embodiments, the administration is nasal or inhalation. In some embodiments, the administration is inhalation. In some embodiments, the administration is once daily, weekly, biweekly or monthly. In some embodiments, the administration comprises administration of an effective dose of from about 0.01 to about 10 mg/kg of the mRNA in the pharmaceutical composition. In some embodiments, the administration increases expression of CFTR in the lung epithelium.

The present disclosure also provides a method of expressing a CFTR protein in a cell comprising contacting the cell with a pharmaceutical composition of any one of the foregoing embodiments.

The present disclosure also provides for a kit for expressing a human CFTR in vivo, the kit comprising a pharmaceutical composition of any one of the preceding embodiments and a device for administering a dose. In some embodiments, the dose is an effective dose of from about 0.01 to about 10 mg/kg of the mRNA in the pharmaceutical composition. In some embodiments, the device comprises an injection needle, an intravenous needle, or an inhalation device. In some embodiments, the device is an inhalation device.

### Pharmaceutical Compositions and Delivery Methods

To facilitate expression of mRNA in vivo, the nucleic acid lipid formulation delivery vehicles described herein can be combined with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. Preferably, the nucleic acid lipid formulation is a CFTR mRNA-lipid nanoparticle formulation as described herein. Preferably, the mRNA encodes a human CFTR protein of SEQ ID NOs: 93 or 99, preferably formulated in a lipid delivery system or lipid carrier and preferably comprising pharmaceutically acceptable excipients. In some embodiments, the pharmaceutical composition further comprises pharmaceutically acceptable excipients. Pharmaceutical compositions disclosed herein preferably facilitate expression of CFTR mRNA in vivo.

The lipid formulations and pharmaceutical compositions of the present disclosure may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

The pharmaceutical compositions described herein can achieve expression of a CFTR protein in the lung epithelial cells of a subject. Suitable routes of administration include, for example, intratracheal, inhaled, or intranasal. In some embodiments, the administration results in delivery of the mRNA to a lung epithelial cell. In some embodiments, the administration shows a selectivity towards lung epithelial cells over other types of lung cells and cells of the airways.

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or mRNA); (4) alter the biodistribution (e.g., target the polynucleotide, primary construct, or mRNA to specific tissues or cell types); (5) increase the translation of encoded protein in vivo; and/or (6) alter the release profile of encoded protein in vivo.

Preferably, mRNAs and lipid formulations thereof may be administered in a local rather than systemic manner. Local delivery can be affected in various ways, depending on the tissue to be targeted. For example, aerosols containing compositions of the present disclosure can be inhaled (for nasal, tracheal, or bronchial delivery).

Pharmaceutical compositions may be administered to any desired tissue. In some embodiments, the CFTR mRNA delivered by a lipid formulation or composition of the present disclosure is expressed in the tissue in which the lipid formulation and/or composition was administered. In some embodiments, the mRNA delivered is expressed in a tissue different from the tissue in which the lipid formulation and/or composition was administered. Example tissues in which delivered mRNA may be delivered and/or expressed include, but are not limited to the lung, trachea, and/or nasal passages.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or mRNA (e.g., for transplantation into a subject), hyaluronidase, nanoparticle mimics and combinations thereof.

Accordingly, the formulations described herein can include one or more excipients, each in an amount that together increases the stability of the nucleic acid in the lipid formulation, increases cell transfection by the nucleic acid (e.g., mRNA), increases the expression of the encoded protein, and/or alters the release profile of the encoded protein. Further, the mRNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

A dosage form of the composition of this disclosure can be solid, which can be reconstituted in a liquid prior to administration. The solid can be administered as a powder. In some embodiments, the pharmaceutical composition comprises a nucleic acid lipid formulation that has been lyophilized.

In a preferred embodiment, the dosage form of the pharmaceutical compositions described herein can be a liquid suspension of CFTR mRNA lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about −70° C. In some embodiments, the suspension is diluted with sterile water prior to inhalable administration. In some embodiments, inhalable administration comprises diluting the suspension with about 1 volume to about 4 volumes of sterile water. In some embodiments, a lyophilized CFTR-mRNA lipid nanoparticle formulation can be resuspended in a buffer as described herein.

The compositions and methods of the disclosure may be administered to subjects by a variety of mucosal administration modes, including intranasal and/or intrapulmonary. In some aspects of this disclosure, the mucosal tissue layer includes an epithelial cell layer. The epithelial cell can be pulmonary, tracheal, bronchial, alveolar, nasal, and/or buccal. Compositions of this disclosure can be administered using conventional actuators such as mechanical spray devices, as well as pressurized, electrically activated, or other types of actuators.

The mRNA compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, for example, aerosolized, atomized, or nebulized. Particles of the composition, spray, or aerosol can be in either a liquid or solid form, for example, a lyophilized lipid formulation. Preferred systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in TRANSDERMAL SYSTEMIC MEDICATION, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the CFTR mRNA lipid formulation or suspended in a pharmaceutical solvent, e.g., water, ethanol, or mixtures thereof.

Nasal and pulmonary spray solutions of the present disclosure typically comprise the drug or drug to be delivered, optionally formulated with a surface-active agent, such as a nonionic surfactant (e.g., polysorbate-80), and one or more buffers, provided that the inclusion of the surfactant does not disrupt the structure of the lipid formulation. In some embodiments of the present disclosure, the nasal spray solution further comprises a propellant. The pH of the nasal spray solution may be from pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure provides a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet, or gel.

To formulate compositions for pulmonary delivery within the present disclosure, the CFTR mRNA lipid formulation can be combined with various pharmaceutically acceptable additives, as well as a base or carrier for dispersion of the CFTR mRNA lipid formulation(s). Examples of additives include pH control agents such as arginine, sodium hydroxide, glycine, hydrochloric acid, citric acid, and mixtures thereof Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of ⅓ to 3, more typically ½ to 2, and most often ¾ to 1.7.

The CFTR mRNA lipid formulation may be dispersed in abase or vehicle, which may comprise a hydrophilic compound having a capacity to disperse the CFTR mRNA lipid formulation and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the CFTR mRNA lipid formulation.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the CFTR mRNA lipid formulation may be administered in a time release formulation, for example in a composition which includes a slow release polymer. The CFTR mRNA lipid formulation can be prepared with carriers that will protect against rapid release, for example a controlled release vehicle such as a polymer, microencapsulated delivery system, or a bioadhesive gel. Prolonged delivery of the CFTR mRNA lipid formulation, in various compositions of the disclosure can be brought about by including in the composition agents that delay absorption, for example, aluminum monostearate hydrogels and gelatin.

It has been demonstrated that nucleic acids can be delivered to the lungs by intratracheal administration of a liquid suspension of the nucleic acid composition and inhalation of an aerosol mist produced by a liquid nebulizer or the use of a dry powder apparatus such as that described in U.S. Pat. No. 5,780,014, incorporated herein by reference.

In certain embodiments, the compositions of the disclosure may be formulated such that they may be aerosolized or otherwise delivered as a particulate liquid or solid prior to or upon administration to the subject. Such compositions may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.5 mg/kg of mRNA per dose) to the subject. For example, in certain embodiments, the compositions of the disclosure are administered to a subject using a metered dose inhaler containing a suspension or solution comprising the composition and a suitable propellant. In certain embodiments, the compositions of the disclosure may be formulated as a particulate powder (e.g., respirable dry particles) intended for inhalation. In certain embodiments, compositions of the disclosure formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller). In yet other embodiments, the compositions of the disclosure are formulated to include one or more pulmonary surfactants (e.g., lamellar bodies). In some embodiments, the compositions of the disclosure are administered to a subject such that a concentration of at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compositions of the disclosure are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg mRNA is administered in one or more doses. The values and ranges recited herein include any subvalue or subrange therebetween.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

According to the present disclosure, a therapeutically effective dose of the provided composition, when administered regularly, results in an increased CFTR protein expression or activity level in a subject as compared to a baseline CFTR protein expression or activity level before treatment. Typically, the CFTR protein expression or activity level is measured in a biological sample obtained from the subject such as blood, plasma or serum, urine, or solid tissue extracts. The baseline level can be measured immediately before treatment. In some embodiments, administering a pharmaceutical composition described herein results in an increased CFTR protein expression or activity level in a biological sample (e.g., plasma/serum or lung epithelial swab) by at least about 10%, 20%, 30%, 40%, 50%, 60%, 70%, 80%, 90%, or 95% as compared to a baseline level before treatment. In some embodiments, administering the provided composition results in an increased CFTR protein expression or activity level in a biological sample (e.g., plasma/serum or lung epithelial swab) by at least about 10%, 20%, 30%, 40%, 50%, 60%, 70%, 80%, 90%, or 95% as compared to a baseline level before treatment for at least about 24 hours, at least about 48 hours, at least about 72 hours, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days.

### Treatment of Cystic Fibrosis

The compositions of the present disclosure can be used for treating cystic fibrosis. In some embodiments, the present disclosure provides a method of treating cystic fibrosis by administering to a subject in need of treatment an mRNA encoding a CFTR protein as described herein or a pharmaceutical composition containing the mRNA. The mRNA or a pharmaceutical composition containing the mRNA may be administered directly to the lung of the subject. Various administration routes for pulmonary delivery may be used. In some embodiments, an mRNA or a composition containing an mRNA described herein is administered by inhalation, nebulization or aerosolization. In various embodiments, administration of the mRNA results in expression of CFTR in the lung of the subject (e.g., epithelial cells of the lung).

In a particular embodiment, the present disclosure provides a method of treating cystic fibrosis by administering to the lung of a subject in need of treatment an mRNA comprising a coding sequence which encodes SEQ ID NO:93. In certain embodiments, the present disclosure provides a method of treating cystic fibrosis by administering to the lung of a subject in need of treatment an mRNA comprising a coding sequence which encodes an amino acid sequence at least about 70%, 75%, 80%, 85%, 90%, 95%, 96%, 97%, 98%, 99%, or 100% identical to SEQ ID NO: 93. In another particular embodiment, the present disclosure provides a method of treating cystic fibrosis by administering to the lung of a subject in need of treatment an mRNA comprising a coding sequence of SEQ ID NOs: 100-105. In other embodiments, the present disclosure provides a method of treating cystic fibrosis by administering to the lung of a subject in need of treatment an mRNA comprising a coding sequence at least 65%, 70%, 75%, 80%, 85%, 90%, 95%, 96%, 97%, 98%, 99%, or 100% identical to SEQ ID NOs: 100-105.

### CFTR Mutation Classes

The pharmaceutical compositions and methods described herein can be used to treat a patient suffering from CF in any of its classes. These classes are described below.

Class 1A (No mRNA): The first class of mutations keeps the mRNA from even being synthesized. When a protein is going to be made in a cell, an enzyme called RNA polymerase binds to a region in the DNA called a promoter. The promoter is usually located right before the section of DNA that codes for a specific protein. If the promoter for CFTR contains a mutation, it can lead to the RNA polymerase not being able to bind to the DNA and therefore not transcribe the gene into mRNA. The end result is no CFTR protein being produced at all. Examples of mutations that lead to no CFTR mRNA include the Dele2,3(21 kb) and 1717-1G→A. No therapy is currently available to correct this type of mutation. However, there is some research into treatments to inhibit sodium channels or stimulate other chloride protein channels at the cell surface to balance ion levels without the need for the CFTR protein.

Class 1B (No Protein): In this class of mutations, the CFTR mRNA is produced but is damaged and cannot be made into protein. There is a specific sequence in the DNA that is then carried over to the RNA, which signals to the ribosome to stop reading the message and marks the end of protein production. Sometimes, because of a mutation, one of these stop sequences appears too early in the mRNA. This results in the production of a shortened version of the CFTR protein, which is then degraded by the cell. Gly542X and Trp1282X are types of class 1B mutations. Read-through compounds can help the ribosome skip over the early stop sequence, read the rest of the information on the mRNA, and produce CFTR proteins. Ataluren was one such compound being investigated as a potential treatment for this kind of mutation but its development ended due to failed Phase 3 clinical trial results.

Class 2 (No Traffic): In this class of mutations, the CFTR protein is made but fails to reach the cell membrane. The CFTR protein has 1,480 amino acids in it and sometimes even a single error can cause the protein to misfold. The cell will often stop misfolded proteins from going to the cell surface and will destroy them. Examples of class 2 mutations include Phe508del, Asn1303Lys, and Ala561Glu. To correct the misfolded proteins and help them reach the cell membrane, treatments called CFTR correctors can be used. Some examples of CFTR correctors include lumacaftor/ivacaftor (marketed as Orkambi) and tezacaftor/ivacaftor (marketed as Symdeko), both produced by Vertex Pharmaceuticals.

Class 3 (Impaired Gating): Another type of mutation can result in the production of a CFTR protein that makes it to the cell membrane but does not open correctly. This is often referred to as a “gating defect.” Gly551Asp, Ser549Arg, and Gly1349Asp are examples of mutations causing gating defects. Treatments called CFTR potentiators, such as Kalydeco, can be used to open the channels and/or keep them open for longer.

Class 4 (Decreased Conductance): The fourth class of mutation results in a CFTR protein that makes it to the cell membrane and reacts to cell signaling to open, but the protein is misshapen and only allows a small amount of chloride ions to pass through. This reduction in chloride ion movement is called decreased conductance. Examples of such mutations include Arg117His, Arg334Trp, and Ala455Glu. CFTR potentiators can also be helpful for these mutations to keep the channels open for longer to allow more chloride ions to flow through.

Class 5 (Less Protein): Sometimes a mutation can lead to CFTR protein being produced but just not in sufficient amounts. This is often caused by a process called alternative splicing in which correct versions of the protein are sometimes made but more often incorrect versions are produced. The incorrect versions never make it to the cell surface, which leads to a reduction in the number of CFTR protein channels at the cell membrane. Class 5 mutations include 3272-26A→G, 3849+10 kg C→T. Possible treatments for this type of mutation include CFTR correctors to correct the misshapen CFTR proteins, CFTR potentiators to try and keep the working CFTR proteins open for longer, CFTR amplifiers to increase the amount of mRNA and therefore more CFTR protein being produced, or antisense oligonucleotides, which can have a number of different uses.

Class 6 (Less Stable Protein): The final type of mutation can result in a working CFTR protein, but the protein configuration is not stable and will degrade too quickly once on the cell surface. Class 6 mutations include c. 120del123 and rPhe580del. Stabilizers are a class of treatment for this type of mutation. They work to inhibit enzymes that break down CFTR. A treatment called cavosonstat was being investigated for this use but failed to meet primary objectives in a Phase 2 clinical trial.

In some embodiments, a CFTR mRNA lipid formulation or a pharmaceutical composition comprising the same is used to treat a patient having a Class 1A mutation. In some embodiments, a CFTR mRNA lipid formulation or a pharmaceutical composition comprising the same is used to treat a patient having a Class 1B mutation. In some embodiments, a CFTR mRNA lipid formulation or a pharmaceutical composition comprising the same is used to treat a patient having a Class 2 mutation. In some embodiments, a CFTR mRNA lipid formulation or a pharmaceutical composition comprising the same is used to treat a patient having a Class 3 mutation. In some embodiments, a CFTR mRNA lipid formulation or a pharmaceutical composition comprising the same is used to treat a patient having a Class 4 mutation. In some embodiments, a CFTR mRNA lipid formulation or a pharmaceutical composition comprising the same is used to treat a patient having a Class 5 mutation. In some embodiments, a CFTR mRNA lipid formulation or a pharmaceutical composition comprising the same is used to treat a patient having a Class 6 mutation.

### Combinations

The CFTR mRNA, formulations thereof, or encoded CFTR proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, mRNA disclosed herein and preferably an mRNA sequence comprising SEQ ID NO: 49, 53, 66, 68, 69, 72, or 100-105 encoding a CFTR protein of SEQ ID NO: 99 may be used in combination with a pharmaceutical agent for the treatment of CFTR deficiency. The pharmaceutical agent includes, but is not limited to one or more of: Trikafta® (Elexacaftor, ivacaftor, tezacaftor, marketed by Vertex Pharmaceuticals), Symdeko® (tezacaftor and ivacaftor, Vertex), Orkambi® (lumacaftor and ivacaftor, Vertex), Kalydeco® (ivacaftor, Vertex), compositions and agents for airway clearance, antibiotics, anti-inflammatory agents, bronchodilators, mucus thinners, etc. Multiple vitamins, calcium supplements or combined with a low protein/high caloric diet regimen. In general, it is expected that agents utilized in combination with the presently disclosed CFTR mRNA and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### Definitions

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

The phrases “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

The term “acyl,” as used herein, represents a hydrogen or an alkyl group (e.g., a haloalkyl group), as defined herein, that is attached to the parent molecular group through a carbonyl group, as defined herein, and is exemplified by formyl (i.e., a carboxyaldehyde group), acetyl, trifluoroacetyl, propionyl, butanoyl and the like. Example unsubstituted acyl groups include from 1 to 7, from 1 to 11, or from 1 to 21 carbons. In some embodiments, the alkyl group is further substituted with 1, 2, 3, or 4 substituents as described herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the example alkyl substituent groups described herein.

The term “alkoxy” represents a chemical substituent of formula OR, where R is a C₁₋₂₀ alkyl group (e.g., C₁₋₆ or C₁₋₁₀ alkyl), unless otherwise specified. Example alkoxy groups include methoxy, ethoxy, propoxy (e.g., n-propoxy and isopropoxy), t-butoxy, and the like. In some embodiments, the alkyl group can be further substituted with 1, 2, 3, or 4 substituent groups as defined herein (e.g., hydroxy or alkoxy).

The term “alkoxyalkyl” represents an alkyl group that is substituted with an alkoxy group. Example unsubstituted alkoxyalkyl groups include between 2 to 40 carbons (e.g., from 2 to 12 or from 2 to 20 carbons, such as C₁₋₆ alkoxy-C₁₋₆ alkyl, C₁₋₁₀ alkoxy-C₁₋₁₀ alkyl, or C₁₋₂₀ alkoxy-C₁₋₂₀ alkyl). In some embodiments, the alkyl and the alkoxy each can be further substituted with 1, 2, 3, or 4 substituent groups as defined herein for the respective group.

The term “alkoxycarbonyl,” as used herein, represents an alkoxy, as defined herein, attached to the parent molecular group through a carbonyl atom (e.g., C(O)—OR, where R is H or an optionally substituted C₁₋₆, C₁₋₁₀, or C₁₋₂₀ alkyl group). Example unsubstituted alkoxycarbonyl include from 1 to 21 carbons (e.g., from 1 to 11 or from 1 to 7 carbons). In some embodiments, the alkoxy group is further substituted with 1, 2, 3, or 4 substituents as described herein.

The term “alkoxycarbonylalkyl,” as used herein, represents an alkyl group, as defined herein, that is substituted with an alkoxycarbonyl group, as defined herein (e.g., -alkyl-C(O)—OR, where R is an optionally substituted C₁₋₂₀, C₁₋₁₀, or C₁₋₆ alkyl group). Example unsubstituted alkoxycarbonylalkyl include from 3 to 41 carbons (e.g., from 3 to 10, from 3 to 13, from 3 to 17, from 3 to 21, or from 3 to 31 carbons, such as C₁₋₆ alkoxycarbonyl-C₁₋₆ alkyl, C₁₋₁₀ alkoxycarbonyl-C₁₋₁₀ alkyl, or C₁₋₂₀ alkoxycarbonyl-C₁₋₂₀ alkyl). In some embodiments, each alkyl and alkoxy group is further independently substituted with 1, 2, 3, or 4 substituents as described herein (e.g., a hydroxy group).

The term “alkoxycarbonylalkenyl,” as used herein, represents an alkenyl group, as defined herein, that is substituted with an alkoxycarbonyl group, as defined herein (e.g., -alkenyl-C(O)—OR, where R is an optionally substituted C₁₋₂₀, C₁₋₁₀, or C₁₋₆ alkyl group). Example unsubstituted alkoxycarbonylalkenyl include from 4 to 41 carbons (e.g., from 4 to 10, from 4 to 13, from 4 to 17, from 4 to 21, or from 4 to 31 carbons, such as C₁₋₆ alkoxycarbonyl-C₂₋₆ alkenyl, C₁₋₁₀ alkoxycarbonyl-C₂₋₁₀ alkenyl, or C₁₋₂₀ alkoxycarbonyl-C₂₋₂₀ alkenyl). In some embodiments, each alkyl, alkenyl, and alkoxy group is further independently substituted with 1, 2, 3, or 4 substituents as described herein (e.g., a hydroxy group).

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The term “lower alkyl” means a group having one to six carbons in the chain which chain may be straight or branched. Non-limiting examples of suitable alkyl groups include methyl, ethyl, n-propyl, isopropyl, n-butyl, t-butyl, n-pentyl, and hexyl.

The term “alkylsulfinyl,” as used herein, represents an alkyl group attached to the parent molecular group through an S(O) group. Example unsubstituted alkylsulfinyl groups are from 1 to 6, from 1 to 10, or from 1 to 20 carbons. In some embodiments, the alkyl group can be further substituted with 1, 2, 3, or 4 substituent groups as defined herein.

The term “alkylsulfinylalkyl,” as used herein, represents an alkyl group, as defined herein, substituted by an alkylsulfinyl group. Example unsubstituted alkylsulfinylalkyl groups are from 2 to 12, from 2 to 20, or from 2 to 40 carbons. In some embodiments, each alkyl group can be further substituted with 1, 2, 3, or 4 substituent groups as defined herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the example alkyl substituent groups described herein.

The term “amidine,” as used herein, represents a —C(═NH)NH₂ group.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkylcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkylheterocyclyl (e.g., alkylheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the disclosure can be an unsubstituted amino (i.e., —NH₂) or a substituted amino (i.e., —N(R′)₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₁₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). In some embodiments, the amino acid is attached to the parent molecular group by a carbonyl group, where the side chain or amino group is attached to the carbonyl group. Example side chains include an optionally substituted alkyl, aryl, heterocyclyl, alkylaryl, alkylheterocyclyl, aminoalkyl, carbamoylalkyl, and carboxyalkyl. Example amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alkyl-C₆₋₁₀ aryl; (16) SO₂R^{D′}, where R^{D′} is selected from the group consisting of (a) C₁₋₆ alkyl, (b) C₆₋₁₀ aryl, (c) C₁₋₆ alkyl-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alkyl-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each RN is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each RN is, independently, hydrogen or optionally substituted C₁₋₆ alkyl; and (21) amidine. In some embodiments, each of these groups can be further substituted as described herein.

The term “aminoalkyl,” as used herein, represents an alkyl group, as defined herein, substituted by an amino group, as defined herein. The alkyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “aminoalkenyl,” as used herein, represents an alkenyl group, as defined herein, substituted by an amino group, as defined herein. The alkenyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “anionic lipid” means a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The terms “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The term “boranyl,” as used herein, represents —B(R^{B1})₃, where each R^{B1} is, independently, selected from the group consisting of H and optionally substituted alkyl. In some embodiments, the boranyl group can be substituted with 1, 2, 3, or 4 substituents as defined herein for alkyl.

The term “boranophosphate” has the ordinary meaning as understood in the art and can include protonated, deprotonated, and tautomeric forms thereof. For example, a boranophosphate within the context of a compound can have the structure

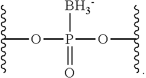

The term “biocompatible” means compatible with living cells, tissues, organs or systems posing little to no risk of injury, toxicity or rejection by the immune system.

The term “biodegradable” means capable of being broken down into innocuous products by the action of living things.

The phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present disclosure may be considered biologically active if even a portion of the polynucleotide is biologically active or mimics an activity considered biologically relevant.

The terms “carbocyclic” and “carbocyclyl,” as used herein, refer to an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbamoyl,” as used herein, represents —C(O)—N(R^{N1})₂, where the meaning of each R^{N1} is found in the definition of “amino” provided herein.

The term “carbamoylalkyl,” as used herein, represents an alkyl group, as defined herein, substituted by a carbamoyl group, as defined herein. The alkyl group can be further substituted with 1, 2, 3, or 4 substituent groups as described herein.

The term “carbamyl,” as used herein, refers to a carbamate group having the structure —NR^{N1}C(═O)OR or —OC(═O)N(R^{N1})₂, where the meaning of each R^{N1} is found in the definition of “amino” provided herein, and R is alkyl, cycloalkyl, alkylcycloalkyl, aryl, alkylaryl, heterocyclyl (e.g., heteroaryl), or alkylheterocyclyl (e.g., alkylheteroaryl), as defined herein.

The term “carbonyl,” as used herein, represents a C(O) group, which can also be represented as C═O.

The term “carboxyaldehyde” represents an acyl group having the structure —C(O)H.

The term “carboxy,” as used herein, means —CO₂H.

The term “cationic lipid” means amphiphilic lipids and salts thereof having a positive, hydrophilic head group; one, two, three, or more hydrophobic fatty acid or fatty alkyl chains; and a connector between these two domains. An ionizable or protonatable cationic lipid is typically protonated (i.e., positively charged) at a pH below its pKa and is substantially neutral at a pH above the pKa. Preferred ionizable cationic lipids are those having a pKa that is less than physiological pH, which is typically about 7.4. The cationic lipids of the disclosure may also be termed titratable cationic lipids. The cationic lipids can be an “amino lipid” having a protonatable tertiary amine (e.g., pH-titratable) head group. Some amino exemplary amino lipid can include C₁₈ alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, 7-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA)(also known as 1-Bl 1).

The term “comprising” is intended to be open and permits but does not require the inclusion of additional elements or steps. When the term “comprising” is used herein, the term “consisting of” is thus also encompassed and disclosed.

The term “composition” means a product comprising the specified ingredients in the specified amounts, as well as any product that results, directly or indirectly, from combination of the specified ingredients in the specified amounts.

The term “in combination with” means the administration of a lipid formulated mRNA of the present disclosure with other medicaments in the methods of treatment of this disclosure, means-that the lipid formulated mRNA of the present disclosure and the other medicaments are administered sequentially or concurrently in separate dosage forms, or are administered concurrently in the same dosage form.

The term “commercially available chemicals” and the chemicals used in the Examples set forth herein may be obtained from standard commercial sources, where such sources include, for example, Acros Organics (Pittsburgh, Pa.), Sigma-Adrich Chemical (Milwaukee, Wis.), Avocado Research (Lancashire, U.K.), Bionet (Cornwall, U.K.), Boron Molecular (Research Triangle Park, N.C.), Combi-Blocks (San Diego, Calif.), Eastman Organic Chemicals, Eastman Kodak Company (Rochester, N.Y.), Fisher Scientific Co. (Pittsburgh, Pa.), Frontier Scientific (Logan, Utah), ICN Biomedicals, Inc. (Costa Mesa, Calif.), Lancaster Synthesis (Windham, N.H.), Maybridge Chemical Co. (Cornwall, U.K.), Pierce Chemical Co. (Rockford, Ill.), Riedel de Haen (Hannover, Germany), Spectrum Quality Product, Inc. (New Brunswick, N.J.), TCI America (Portland, Oreg.), and Wako Chemicals USA, Inc. (Richmond, Va.).

The phrase “compounds described in the chemical literature” may be identified through reference books and databases directed to chemical compounds and chemical reactions, as known to one of ordinary skill in the art. Suitable reference books and treatise that detail the synthesis of reactants useful in the preparation of compounds disclosed herein, or provide references to articles that describe the preparation of compounds disclosed herein, include for example, “Synthetic Organic Chemistry,” John Wiley and Sons, Inc. New York; S. R. Sandler et al, “Organic Functional Group Preparations,” 2nd Ed., Academic Press, New York, 1983; H. O. House, “Modern Synthetic Reactions,” 2nd Ed., W. A. Benjamin, Inc. Menlo Park, Calif., 1972; T. L. Glichrist, “Heterocyclic Chemistry,” 2nd Ed. John Wiley and Sons, New York, 1992; J. March, “Advanced Organic Chemistry: reactions, Mechanisms and Structure,” 5th Ed., Wiley Interscience, New York, 2001; Specific and analogous reactants may also be identified through the indices of known chemicals prepared by the Chemical Abstract Service of the American Chemical Society, which are available in most public and university libraries, as well as through online databases (the American Chemical Society, Washington, D.C. may be contacted for more details). Chemicals that are known but not commercially available in catalogs may be prepared by custom chemical synthesis houses, where many of the standard chemical supply houses (such as those listed above) provide custom synthesis services.

The term “complementary nucleotide bases” means a pair of nucleotide bases that form hydrogen bonds with each other. Adenine (A) pairs with thymine (T) or with uracil (U) in RNA, and guanine (G) pairs with cytosine (C). Complementary segments or strands of nucleic acid that hybridize (i.e. join by hydrogen bonding) with each other. By “complementary” is meant that a nucleic acid can form hydrogen bond(s) with another nucleic acid sequence either by traditional Watson-Crick or by other non-traditional modes of binding.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this disclosure can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁-6 thioalkoxy-C₁₋₆ alkyl); (3) C₁₂ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alkyl-C₆₋₁₀ aryl; (8) azido; (9) C₃₋₈ cycloalkyl; (10) C₁₋₆ alkyl-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alkyl-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alkyl-C₆₋₁₀ aryl; (19) —(CH₂)_{q}SO₂R^{D′}, where q is an integer from zero to four and where RD is selected from the group consisting of (a) C₆₋₁₀ alkyl, (b) C₆₋₁₀ aryl, and (c) C₁₋₆ alkyl-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alkyl-C₁₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alkyl-C₁₋₁₂ heterocyclyl (e.g., C₁₋₆ alkyl-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkyl group of a C₁-alkaryl or a C₁-alkylheterocyclyl can be further substituted with an oxo group to afford the respective aryloyl and (heterocyclyl)oyl substituent group.

The term “diastereomer,” as used herein means stereoisomers that are not mirror images of one another and are non-superimposable on one another.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (Cis), and icosoyl (C₂₀). In preferred embodiments, R¹ and R² are the same, i.e., R¹ and R² are both myristoyl (i.e., dimyristoyl), R¹ and R² are both stearoyl (i.e., distearoyl).

The term “dialkyloxypropyl” or “DAA” includes a compound having 2 alkyl chains, R and R, both of which have independently between 2 and 30 carbons. The alkyl groups can be saturated or have varying degrees of unsaturation.

The term “effective amount” of an agent, as used herein, is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

The term “enantiomer,” as used herein, means each individual optically active form of a compound of the disclosure, having an optical purity or enantiomeric excess (as determined by methods standard in the art) of at least 80% (i.e., at least 90% of one enantiomer and at most 10% of the other enantiomer), preferably at least 90% and more preferably at least 98%.

An “enzyme having cystic fibrosis transmembrane conductance regulator activity”, an “enzyme having CFTR activity”, a “protein having CFTR activity”, a “protein having cystic fibrosis transmembrane conductance regulator activity”, a “CFTR enzyme”, or a “CFTR protein” means a protein or enzyme that conducts chloride ions across epithelial cell membranes and helps to maintain the balance of salt and water on the epithelial surfaces of the body. The CFTR protein is a particular type of protein called an ion channel, which has a tubular shape and moves atoms or molecules that have an electrical charge from inside the cell to outside or from outside the cell to inside. In the lung, the CFTR ion channel moves chloride ions from inside the cell to outside the cell. To get out of the cell, the chloride ions move through the center of the tube formed by the CFTR protein. Once the chloride ions are outside the cell, they attract a layer of water. This water layer is important because it allows cilia on the surface of the lung cells, to sweep back and forth. This sweeping motion moves mucus up and out of the airways.

The term “fully encapsulated” means that the nucleic acid (e.g., mRNA) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” also means that the nucleic acid-lipid particles do not rapidly decompose into their component parts upon in vivo administration.

The terms “halo” and “Halogen”, as used herein, represents a halogen selected from bromine, chlorine, iodine, or fluorine.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls (e.g., —CF₃), —CHF₂, —CH₂F, —CCl₃, —CH₂CH₂Br, —CH₂CH(CH₂CH₂Br)CH₃, and —CHICH₃. In some embodiments, the haloalkyl group can be further substituted with 1, 2, 3, or 4 substituent groups as described herein for alkyl groups.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups.

The term “hydrocarbon,” as used herein, represents a group consisting only of carbon and hydrogen atoms.

The term “hydroxy,” as used herein, represents an —OH group. In some embodiments, the hydroxy group can be substituted with 1, 2, 3, or 4 substituent groups (e.g., O-protecting groups) as defined herein for an alkyl.

The term “hydroxyalkenyl,” as used herein, represents an alkenyl group, as defined herein, substituted by one to three hydroxy groups, with the proviso that no more than one hydroxy group may be attached to a single carbon atom of the alkyl group, and is exemplified by dihydroxypropenyl, hydroxyisopentenyl, and the like. In some embodiments, the hydroxyalkenyl group can be substituted with 1, 2, 3, or 4 substituent groups (e.g., O-protecting groups) as defined herein for an alkyl.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like. In some embodiments, the hydroxyalkyl group can be substituted with 1, 2, 3, or 4 substituent groups (e.g., O-protecting groups) as defined herein for an alkyl.

The term “hydrate” means a solvate wherein the solvent molecule is H₂O.

The term “isomer,” as used herein, means any tautomer, stereoisomer, enantiomer, or diastereomer of any compound of the disclosure. It is recognized that the compounds of the disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein, and therefore the compounds of the disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “nitro,” as used herein, represents an —NO₂ group.

The term “N/P ratio” as used herein refers to the ratio of the number of positively charged amine groups (N) of cationic lipids to the number of negatively charged phosphate groups (P) of a CFTR mRNA that is encapsulated, or targeted for encapsulation by, the cationic lipid(s).

The term “nucleic acid” means deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

The term “oxo” as used herein, represents ═O.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present disclosure may exist in different tautomeric forms, all of the latter being included within the scope of the present disclosure.

The term “sulfonyl,” as used herein, represents an —S(O)₂— group.

The term “compound,” is meant to include all stereoisomers, geometric isomers, tautomers, and isotopes of the structures depicted.

The term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

The term “cyclic” refers to the presence of a continuous loop. Cyclic molecules need not be circular, only joined to form an unbroken chain of subunits. Cyclic molecules such as the mRNA of the present disclosure may be single units or multimers or comprise one or more components of a complex or higher order structure.

The term “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

The term “delivery” refers to the act or manner of delivering a compound, substance, entity, moiety, cargo or payload.

The term “delivery agent” refers to any substance which facilitates, at least in part, the in vivo delivery of a polynucleotide to targeted cells.

The term “digest” means to break apart into smaller pieces or components. When referring to polypeptides or proteins, digestion results in the production of peptides.

The term “distal” means situated away from the center or away from a point or region of interest.

The phrase “encoded protein cleavage signal” refers to the nucleotide sequence which encodes a protein cleavage signal.

The term “engineered” refers to a molecule designed to have a feature or property, whether structural or chemical, that varies from a starting point, wild type or native molecule.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

The term “feature” refers to a characteristic, a property, or a distinctive element.

The term “fragment,” as used herein, refers to a portion. For example, fragments of proteins may comprise polypeptides obtained by digesting full-length protein isolated from cultured cells.

The term “functional” biological molecule is a biological molecule in a form in which it exhibits a property and/or activity by which it is characterized.

The term “homology” refers to the overall relatedness between polymeric molecules, e.g. between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

The term “hydrophobic lipids” means compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

The term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The term “isolated” refers to a substance or entity that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

The term “lipid” means an organic compound that comprises an ester of fatty acid and is characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids.

The term “lipid delivery vehicle” means a lipid formulation that can be used to deliver a therapeutic nucleic acid (e.g., mRNA) to a target site of interest (e.g., cell, tissue, organ, and the like). The lipid delivery vehicle can be a nucleic acid-lipid particle, which can be formed from a cationic lipid, a non-cationic lipid (e.g., a phospholipid), a conjugated lipid that prevents aggregation of the particle (e.g., a PEG-lipid), and optionally cholesterol. Typically, the therapeutic nucleic acid (e.g., mRNA) may be encapsulated in the lipid portion of the particle, thereby protecting it from enzymatic degradation.

The term “lipid encapsulated” means a lipid particle that provides a therapeutic nucleic acid such as an mRNA with full encapsulation, partial encapsulation, or both. In a preferred embodiment, the nucleic acid (e.g., mRNA) is fully encapsulated in the lipid particle.

The term “lipid conjugate” means a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers, and mixtures thereof PEG or POZ can be conjugated directly to the lipid or may be linked to the lipid via a linker moiety. Any linker moiety suitable for coupling the PEG or the POZ to a lipid can be used including, e.g., non-ester-containing linker moieties and ester-containing linker moieties. In certain preferred embodiments, non-ester-containing linker moieties, such as amides or carbamates, are used.

The term “amphipathic lipid” or “amphiphilic lipid” means the material in which the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

The term “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end of the linker, and to a payload, e.g., a detectable or therapeutic agent, at a second end of the linker. The linker may be of sufficient length as to not interfere with incorporation into a nucleic acid sequence. The linker can be used for any useful purpose, such as to form multimers (e.g., through linkage of two or more polynucleotides) or conjugates, as well as to administer a payload, as described herein. Examples of chemical groups that can be incorporated into the linker include, but are not limited to, alkyl, alkenyl, alkynyl, amido, amino, ether, thioether, ester, alkyl, heteroalkyl, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond, which can be cleaved for example by the use of tris(2-carboxyethyl)phosphine (TCEP), or other reducing agents, and/or photolysis, as well as an ester bond, which can be cleaved for example by acidic or basic hydrolysis.

The term “mammal” means a human or other mammal or means a human being.

The term “messenger RNA” (mRNA) refers to any polynucleotide which encodes a protein or polypeptide of interest and which is capable of being translated to produce the encoded protein or polypeptide of interest in vitro, in vivo, in situ or ex vivo.

The term “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they may differ from the chemical structure of the A, C, G, U ribonucleotides.

The phrase “nasal potential difference” is used to measure the voltage across the nasal epithelium, which results from transepithelial ion transport and reflects in part CFTR function. The electrophysiologic abnormality in cystic fibrosis was first described 30 years ago and correlates with features of the CF phenotype.

The term “naturally occurring” means existing in nature without artificial aid.

The term “nonhuman vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

The term “nucleotide” means natural bases (standard) and modified bases well known in the art. Such bases are generally located at the 1′ position of a nucleotide sugar moiety. Nucleotides generally comprise a base, sugar, and a phosphate group. The nucleotides can be unmodified or modified at the sugar, phosphate, and/or base moiety, (also referred to interchangeably as nucleotide analogs, modified nucleotides, non-natural nucleotides, non-standard nucleotides and other; see, for example, Usman and McSwiggen, supra; Eckstein, et al., International PCT Publication No. WO 92/07065; Usman, et al., International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include: inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine, thymine and uracil at 1′ position or their equivalents.

The term “off target” refers to any unintended effect on any one or more target, gene, or cellular transcript.

The term “codon-optimized” means a natural (or purposefully designed variant of a natural) coding sequence which has been redesigned by choosing different codons without altering the encoded protein amino acid sequence increasing the protein expression levels (Gustafsson et al, Codon bias and heterologous protein expression. 2004, Trends Biotechnol 22: 346-53). Variables such as high codon adaptation index (CAI), LowU method, mRNA secondary structures, cis-regulatory sequences, GC content and many other similar variables have been shown to somewhat correlate with protein expression levels (Villalobos et al., Gene Designer: a synthetic biology tool for constructing artificial DNA segments. 2006, BMC Bioinformatics 7:285). High CAI (codon adaptation index) method picks a most frequently used synonymous codon for an entire protein coding sequence. The most frequently used codon for each amino acid is deduced from 74218 protein-coding genes from a human genome. The LowU method targets only Li-containing codons that can be replaced with a synonymous codon with fewer U moieties. If there are a few choices for the replacement, the more frequently used codon will be selected. The remaining codons in the sequence are not changed by the LowU method. This method may be used in conjunction with the disclosed mRNAs to design coding sequences that are to be synthesized with 5-methoxy uridine.

The term “open reading frame” or “ORF” to a nucleic acid sequence (DNA or RNA) which is capable of encoding a polypeptide of interest. ORFs often begin with the start codon ATG, and end with a nonsense or termination codon or signal.

The phrase “operably linked” refers to a functional connection between two or more molecules, constructs, transcripts, entities, moieties or the like.

The term “patient” refers to a subject who may seek or be in need of treatment, requires treatment, is receiving treatment, will receive treatment, or a subject who is under care by a trained professional for a particular disease or condition.

The phrase “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “peptide” is less than or equal to 50 amino acids long, e.g., about 5, 10, 15, 20, 25, 30, 35, 40, 45, or 50 amino acids long.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The phrase “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “physicochemical” means of or relating to a physical and/or chemical property.

The term “phosphate” is used in its ordinary sense as understood by those skilled in the art and includes its protonated forms, for example

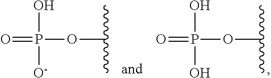 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The term “phosphorothioate” refers to a compound of the general formula

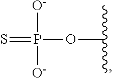

its protonated forms, for example,

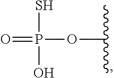 [SMILES: *OP(=O)(O)S]

and its tautomers such as

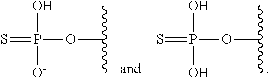 [SMILES: *OP([O-])(O)=S ; *OP(O)(O)=S]

The term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “protein cleavage site” refers to a site where controlled cleavage of the amino acid chain can be accomplished by chemical, enzymatic or photochemical means.

The phrase “protein cleavage signal” refers to at least one amino acid that flags or marks a polypeptide for cleavage.

The term “proteins of interest” or “desired proteins” include those provided herein and fragments, mutants, variants, and alterations thereof.

The terms “purify,” “purified,” “purification” means to make substantially pure or clear from unwanted components, material defilement, admixture or imperfection.

The term “RNA” means a molecule comprising at least one ribonucleotide residue. By “ribonucleotide” is meant a nucleotide with a hydroxyl group at the 2′ position of a β-D-ribo-furanose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA.

The term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

The phrase “signal sequences” refers to a sequence which can direct the transport or localization of a protein.

The terms “significant” or “significantly” are used synonymously with the term “substantially.”

The phrase “single unit dose” is a dose of any therapeutic administered in one dose/at one time/single route/single point of contact, i.e., single administration event.

The term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

The term “solvate” means a physical association of a compound of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

The term “split dose” is the division of single unit dose or total daily dose into two or more doses.

The term “stable” refers to a compound that is sufficiently robust to survive isolation to a useful degree of purity from a reaction mixture, and preferably capable of formulation into an efficacious therapeutic agent.

The terms “stabilize”, “stabilized,” “stabilized region” means to make or become stable.

The term “substituted” means substitution with specified groups other than hydrogen, or with one or more groups, moieties, or radicals which can be the same or different, with each, for example, being independently selected.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The phrase “substantially equal” relates to time differences between doses, the term means plus/minus 2%.

The phrase “substantially simultaneously” relates to plurality of doses, the term means within 2 seconds.

The phrase “suffering from” relates to an individual who is “suffering from” a disease, disorder, and/or condition has been diagnosed with or displays one or more symptoms of a disease, disorder, and/or condition.

The phrase “susceptible to” relates to an individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” means produced, prepared, and/or manufactured by the hand of man. Synthesis of polynucleotides or polypeptides or other molecules of the present disclosure may be chemical or enzymatic.

The term “targeted cells” refers to any one or more cells of interest. The cells may be found in vitro, in vivo, in situ or in the tissue or organ of an organism. The organism may be an animal, preferably a mammal, more preferably a human and most preferably a patient.

The term “therapeutic agent” refers to any agent that, when administered to a subject, has a therapeutic, diagnostic, and/or prophylactic effect and/or elicits a desired biological and/or pharmacological effect.

The term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “total daily dose” is an amount given or prescribed in 24 hour period. It may be administered as a single unit dose.

The term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The term “half-life” is the time required for a quantity such as nucleic acid or protein concentration or activity to fall to half of its value as measured at the beginning of a time period.

The term “in vitro” refers to events that occur in an artificial environment, e.g., in a test tube or reaction vessel, in cell culture, in a Petri dish, etc., rather than within an organism (e.g., animal, plant, or microbe).

The term “in vivo” refers to events that occur within an organism (e.g., animal, plant, or microbe or cell or tissue thereof).

The term “monomer” refers to a single unit, e.g., a single nucleic acid, which may be joined with another molecule of the same or different type to form an oligomer. In some embodiments, a monomer may be an unlocked nucleic acid, i.e., a UNA monomer.

The term “neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” means an amphipathic lipid or a neutral lipid or anionic lipid and is described herein.

The term “oligomer” may be used interchangeably with “polynucleotide” and refers to a molecule comprising at least two monomers and includes oligonucleotides such as DNAs and RNAs. In the case of oligomers containing RNA monomers and/or unlocked nucleic acid (UNA) monomers, the oligomers of the present disclosure may contain sequences in addition to the coding sequence (CDS). These additional sequences may be untranslated sequences, i.e., sequences which are not converted to protein by a host cell. These untranslated sequences can include a 5′ cap, a 5′ untranslated region (5′ UTR), a 3′ untranslated region (3′ UTR), and a tail region, e.g., a poly-A tail region. As described in further detail herein, any of these untranslated sequences may contain one or more UNA monomers—these UNA monomers are not capable of being translated by a host cell's machinery. In the context of the present disclosure, a “mRNA sequence,” a “mRNA sequence,” “translatable polynucleotide,” or “translatable compound” refers to a sequence that comprises a region, e.g., the coding region of an RNA (e.g., the coding sequence of human CFTR or a codon-optimized version thereof), that is capable of being converted to a protein or a fragment thereof, e.g., the human CFTR protein or a fragment thereof.

The terms “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

The term “translatable” may be used interchangeably with the term “expressible” and refers to the ability of polynucleotide, or a portion thereof, to be converted to a polypeptide by a host cell. As is understood in the art, translation is the process in which ribosomes in a cell's cytoplasm create polypeptides. In translation, messenger RNA (mRNA) is decoded by tRNAs in a ribosome complex to produce a specific amino acid chain, or polypeptide. Furthermore, the term “translatable” when used in this specification in reference to an oligomer, means that at least a portion of the oligomer, e.g., the coding region of an oligomer sequence (also known as the coding sequence or CDS), is capable of being converted to a protein or a fragment thereof.

The term “translation efficiency” refers to a measure of the production of a protein or polypeptide by translation of an mRNA sequence in vitro or in vivo. [0080] This disclosure provides a range of mRNA sequence molecules, which can contain one or more UNA monomers, and a number of nucleic acid monomers, wherein the mRNA sequence can be expressible to provide a polypeptide or protein.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

### EXAMPLES

The present disclosure is further described in the following examples, which do not limit the scope of the disclosure described in the claims.

### Example 1: Preparation of hCFTR mRNA Lipid Formulations

This example provides general methods for the preparation of mRNA constructs, mRNA-lipid formulations, and methods for characterizing the same.

### In Vitro Transcription Protocol

Constructs of hCFTR mRNAs were synthesized in vitro using T7RNA polymerase-mediated DNA-dependent RNA transcription. In the transcription reaction, modified and unmodified uridine triphosphates (UTP) were used depending on the desired polynucleotide configuration. Modified UTPs that were used included 5-methoxy-UTP (5MeOU), N¹-methyl pseudo UTP (N1MPU), N¹-methoxy methyl pseudo UTP (N1-MOM), 5-hydroxy methyl UTP, 5-carboxy UTP, and a mixture of modifications using a linearized template for each UTR combination. The mRNA was purified using column chromatography, whereby the DNA template and double stranded RNA contamination of all mRNAs synthesized was removed using an enzymatic reaction. Then, the mRNA was concentrated, and buffer exchanged.

The mRNA constructs also included a 5′ m⁷GpppGm cap and a poly-A tail from about 80 to about 125 adenine nucleotides in length.

Preparation of Lipid Encapsulated mRNA

Lipid encapsulated mRNA particles were prepared by mixing lipids (ionizable cationic lipid: DSPC: Cholesterol: PEG-DMG) in ethanol with different CFTR mRNAs described herein (specific formulations are described in subsequent Examples) dissolved in Citrate buffer. The ionizable cationic lipids used in the formulation were selected lipids of Formula I described hereinabove. The mixed material was instantaneously diluted with Phosphate Buffer. Ethanol was removed by dialysis against phosphate buffer using a regenerated cellulose membrane (100 kD MWCO) or by tangential flow filtration (TFF) using modified polyehtersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 40-60 mM NaCl and 7-12% sucrose, pH 7.3. The formulation was concentrated and followed by a 0.2 μm filtration using PES filters. The mRNA concentration in the formulation was then measured by Ribogreen fluorimetric assay following which the concentration was adjusted to a final desired concentration by diluting with HEPES buffer at pH 7.3 containing 40-60 mM NaCl, 7-12% sucrose, and further containing glycerol. The final formulation was then filtered through a 0.2 μm filter and filled into glass vials, stoppered, capped, and stored at −70±5° C. The frozen formulations were characterized for their mRNA content and percent encapsulation by a RiboGreen assay, mRNA integrity by fragment analyzer, lipid content by high performance liquid chromatography (HPLC), particle size by dynamic light scattering on a Malvern Zetasizer Nano ZS, pH, and osmolality.

### In-Cell Western (ICW)

An In-Cell Western (ICW) assay was developed to assess the potency and ability of the mRNA lipid formulations to transfect cells and express a protein of interest. In the assay, a 96-well collagen plate was used to seed the cells at the appropriate density in Dulbecco's Modified Eagle Medium (DMEM) containing Fetal Bovine Serum (FBS). At the optimal confluence, cells were transfected with the targeted mRNAs diluted in the transfection reagent mix (MessengerMax™ and Opti-MEM®). The cells were placed in a CO₂ incubator and allowed to grow. At the desired timepoint, media was removed, and the cells were fixed in 4% fresh paraformaldehyde (PFA) for 20 min. After that, fixative was removed, and the cells were permeabilized several times in Tris-buffered saline with TWEEN (TBST) for 5 minutes each time. When the permeabilization washes were complete, the cells were incubated with a blocking buffer (ODYSSEY® Blocking Buffer (PBS) (Li-Cor, Lincoln, Nebr.)) for 45 minutes. A primary antibody was then added and incubated for 1 hour at room temperature. The cells were then washed several times in TBST and incubated for 1 hour with a secondary antibody diluted in blocking buffer and containing a CellTag 700 stain. Finally, the cells were washed several times in TBST followed by a last wash in Tris-buffered saline TBS. The plate was imaged using the LI-COR® detection system, and the data was normalized to the total number of cells labeled by the CellTag 700. Specific results of ICW assays are discussed hereinbelow.

### Example 2: Ex Vivo Lung Explant Protocol

Studies were performed to assess the ability of the CFTR mRNA-lipid formulations prepared as described in Example 1 to express in human lungs. This example provides a general description of the materials and methods of the protocol for human lung explant studies.

Human lung explants, both from non-CF individuals and from individuals having CF, were received from the National Development and Research Institutes, Inc. (NDRI). All the handling and processing up to obtaining a slice culture was done under BSL-2 conditions. Briefly, the lungs were wiped out and insufflated with 1.5% low melting point agarose. Then, a conical piece was excised using a coring tool, a block was generated, and 250 μm slices were cut using a slice microtome. The slices were cultured in a Dulbecco's Modified Eagle Medium (DMEM) culture medium. After several washes to remove excess agarose, DMEM culture medium was added, including the proper antibiotics for the lung type being tested (i.e., CF or non-CF), and the slices were cultured with the different CFTR mRNA-lipid formulations as prepared in Example 1. 24 hours post transduction, the slices were homogenized and prepared for Western Blot (WB) analysis. Cell viability measurements were performed using a Lactate Dehydrogenase (LDH) kit (ThermoFisher Scientific) to assure viability of the slices in culture. For the non-CF lungs, the antibiotics included of penicillin and streptomycin. For the CF lungs, the antibiotics included amphotericin, ceftazidime, tobramycin, vancomycin, ciprofloxacin, coly-mycin, sulfamethoxazole, fluconazole, nystatin, antibiotic-antimycotic, tetracycline hydrochloride, rifampicin, and azithromycin.

### Example 3: Screening of CFTR Sequences

Codon-optimized sequences were designed based on the natural human CFTR sequence (hCFTR) and studies were performed to compare the translation efficiency of the various sequences. In these studies, unformulated hCFTR mRNAs were transfected into CF bronchial epithelial (CFBE) cells. CFBE is an immortalized cell line created from the bronchial epithelium of a CF patient homozygous for the F508 deletion. CFBE cells have been used to study CFTR function and response to small molecules due to their clinical relevance to CF and their ability to polarize and form tight junctions.

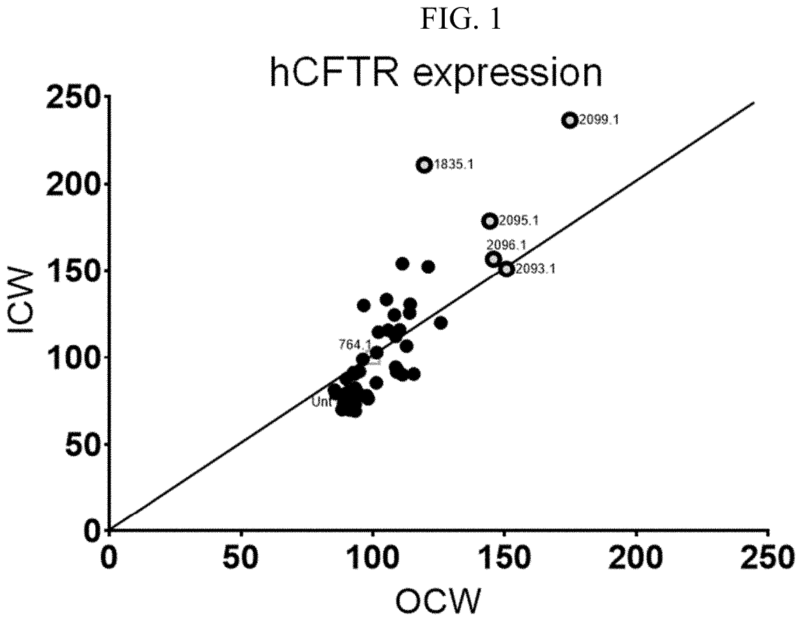

Twenty-four (24) hours post-transfection, expression levels for the various hCFTR mRNAs were determined by In-Cell and On-Cell Western assay (ICW, OCW) using a human CFTR antibody. The correlation between both assays was plotted and the results are shown in FIG. 1. In this figure, “Unt” represents an untransfected negative control, which formed the baseline for this study. The various hCFTR mRNA constructs were ranked based on their respective expression profiles. Highest expressers (grey dots), low-to-medium expressers (black dots), native sequence (grey square) and baseline (Unt) are plotted as shown in FIG. 1. It can be seen that constructs 2099.1 (SEQ ID NO: 72), 1835.1 (SEQ ID NO: 53), 2095.1 (SEQ ID NO: 68), 2096.1 (SEQ ID NO: 69), and 2093.1 (SEQ ID NO: 66) all showed superior expression levels. “0.1” for these constructs indicates that the mRNA was synthesized with 100% of the uridines being N1MPU. The constructs further comprised a 5′ cap and a poly-A tail as described in Example 1.

### Example 4: UTR/ORF Combinations Identified in Transfected CFBE Cells

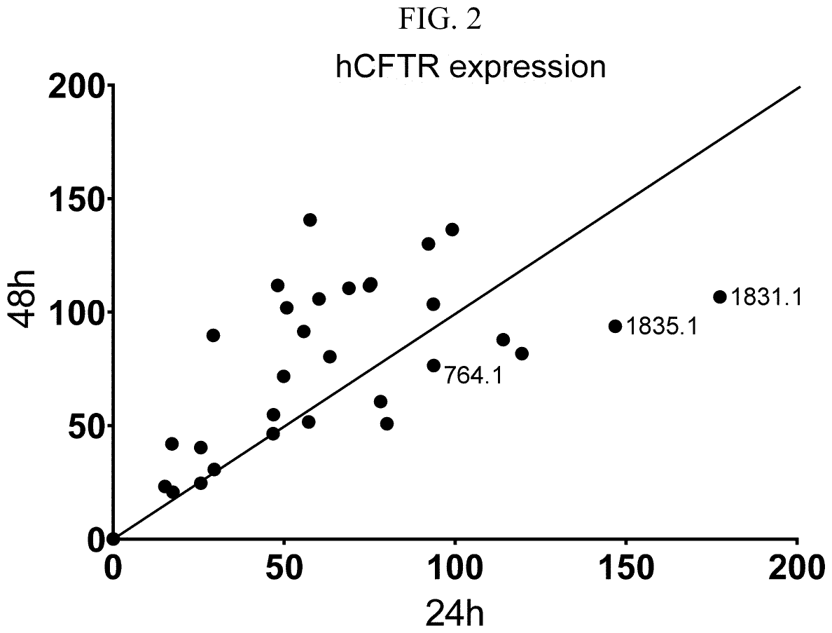

Additional hCFTR constructs were prepared to assess the effect of different UTRs on the expression levels of the hCFTR mRNA. The coding region for each of the hCFTR constructs contained a reference sequence taken from the coding region of SEQ ID NO: 47, which is a commonly used mRNA sequence for wild-type hCFTR that has been slightly changed by introducing a point mutation to remove a cryptic promoter region. (Chow et al., (1997) PNAS 94: 14695-14700). A UTR library was designed for the reference sequence coding region in which selected UTRs were combined with the reference sequence coding region. The unformulated UTR-optimized hCFTR mRNA sequences were then tested in vitro by transfecting CFBE cells. Expression levels were measured at 24 hours and 48 hours post transfection by using a hCFTR specific antibody and the ICW assay described in Example 1. The results are shown in FIG. 2, which is a correlation plot of the expression levels for the various constructs at 24 hours and 48 hours post transfection. In comparison to the negative control of the reference sequence (construct 764.1, also designated SEQ ID NO: 47), it can be seen that the constructs 1835.1 (SEQ ID NO: 53) and 1831.1 (SEQ ID NO: 49) showed especially superior expression levels. These constructs included a 5′ UTR of SEQ ID NO: 106 (TEV), which indicates that this UTR unexpectedly enhances hCFTR expression.

### Example 5: C-Band CFTR Protein Levels In Vitro

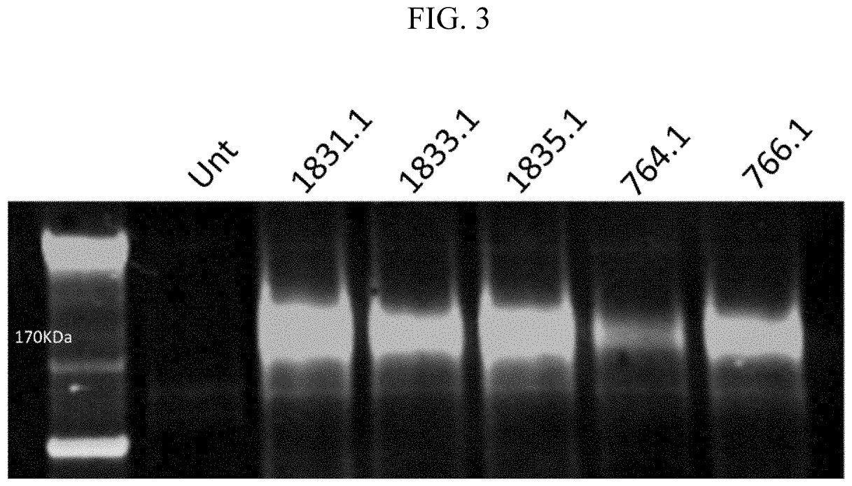

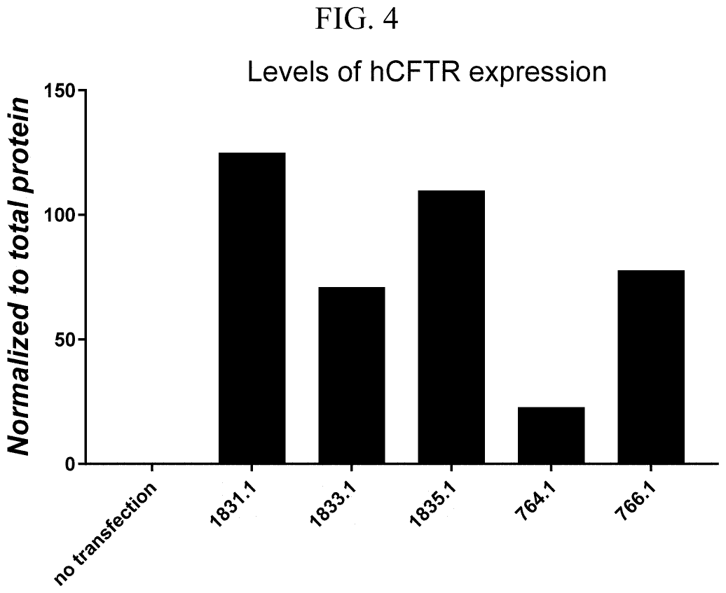

Further studies were conducted to compare the expression of various hCFTR mRNA constructs relative to a reference sequence mRNA. The constructs tested were 1831.1 (SEQ ID NO: 49), 1833.1 (SEQ ID NO: 51), 1835.1 (SEQ ID NO: 53), 766.1 (SEQ ID NO: 48), and the reference sequence construct of 764.1 (wild-type hCFTR mRNA; SEQ ID NO: 47). CFBE cells were transfected with unformulated codon-optimized hCFTR and reference sequence mRNAs, and further analyzed for protein levels using a Western Blot (WB) assay using a primary antibody specific for hCFTR. The results are shown in FIG. 3. The degree of expression was measured by quantifying the C-band (located at a molecular weight of about 170 kDa), which represents a fully-glycosylated, mature CFTR protein, and the results are graphed in FIG. 4. It can be seen in FIGS. 3 and 4 that all codon-optimized hCFTR mRNAs analyzed (SEQ ID NOs: 49, 51, 53, 48), showed higher protein expression levels (more intense signal) over the reference sequence (SEQ ID NO: 47). The lane labeled “Unt” represents the negative control of cells which were untransfected, and as expected no C-band was detected in this sample.

### Example 6: Transfected hCFTR mRNA is Fully Glycosylated

Further experiments were conducted to ensure that the proteins expressed by the hCFTR mRNAs were completely processed into a fully mature CFTR protein. As a membrane bound protein, CFTR's biogenesis carries it through the endoplasmic reticulum (ER) and Golgi apparatus. Within the ER the CFTR polypeptide is core glycosylated at two sites and then within the Golgi apparatus it receives complex glycosylation that is maintained at the level of the plasma membrane. When evaluated on a Western blot the core glycosylated immature form of CFTR migrates further and is designated the “B-band.” The complex glycosylated form of CFTR, representing transit through the Golgi, but not necessarily plasma membrane expression, migrates slower during gel electrophoresis due to its greater molecular weight and is termed “C-band.” Complex glycosylation of the CFTR protein is important as it appears to play a role in prolonging membrane stability. This is supported by the observation that the F508del CFTR protein shows a marked drop in the level of “C-band” as observed in Western blot assays. (J. Cell Sci., 2008. 121(Pt 17): p. 2814-23). Typically, the A-band is observed at a molecular weight of about 130-140 kDa, and corresponds to an immature, incompletely-glycosylated form of CFTR. The B-band is also typical of an incompletely glycosylated (“core glycosylated”) CFTR and is observed at a molecular weight of about 150 kDa. In contrast, the fully mature and glycosylated CFTR protein is identified in the C-band, which corresponds to a molecular weight of about 170 kDa.

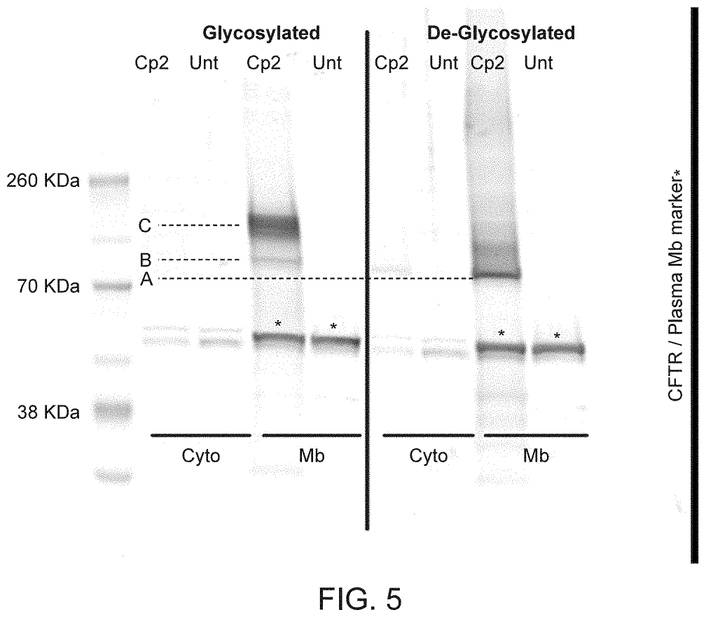

In this study, CFBE cells were transfected with an unformulated codon-optimized hCFTR mRNA (SEQ ID NO: 53). Samples were fractionated into a cytosolic fraction (Cyto) and membrane (Mb) fraction. The fractions of one sample set underwent a deglycosylation process (Deglycosylated) while the fractions of another sample set did not receive this treatment (Glycosylated). The two sample sets were then analyzed for protein expression levels by Western Blot (WB) using a primary antibody specific for hCFTR and for the plasma membrane fraction (sodium potassium ATPase). The results of the WB assay are shown in FIG. 5. As seen in this figure, no hCFTR-specific bands were found in the cytosolic fractions (Cyto) for both the glycosylated and deglycosylated sample sets. hCFTR-specific bands were found in the membrane fractions (Mb) for both sample sets. However, the glycosylated sample set showed a small amount of protein in the B-band, with the majority of protein in the fully-glycosylated C-band and no protein in the A-band, whereas deglycosylated samples showed a significant amount of protein in the unglycosylated A-band and no protein in the B- or C-bands. These results indicate that the codon-optimized hCFTR mRNA is mainly expressing the mature, fully-glycosylated hCFTR protein, with hCFTR completely translocating to the cellular membrane.

### Example 7: Confocal Immunofluorescence of hCFTR In Vitro

Confocal Immunofluorescence microscopy was used to determine whether hCFTR protein expressed by the mRNAs described herein was located in the plasma membrane of transfected cells. In this experiment, CFBE cells were transfected with a codon-optimized hCFTR mRNA (SEQ ID NO: 53; also designated construct 1835.1) and processed for immunofluorescence using an immunofluorescent-antibody probe specific for hCFTR and DAPI as a counterstain. As a negative control, untransfected cells (Unt) were also processed with immunofluorescent-antibody probe specific for hCFTR and DAPI as a counterstain. The DAPI counterstain is indicative of cellular nuclei. The fluorescent images are shown in FIG. 6. In these images, both the untransfected sample (left panel) and the sample transfected with codon-optimized hCFTR mRNA showed several large round structures corresponding to cellular nuclei, as seen by DAPI counterstaining. In contrast, the immunofluorescence associated with the immunofluorescent antibody probe specific for hCFTR showed an even distribution spaced away from the counterstained nuclei only in the image for the hCFTR mRNA transfected cells. This indicates that the hCFTR protein was located in the plasma membrane of transfected cells and agrees with the results described in Example 6.

### Example 8: Dose Response of hCFTR mRNAs in Transfected FRT Cells

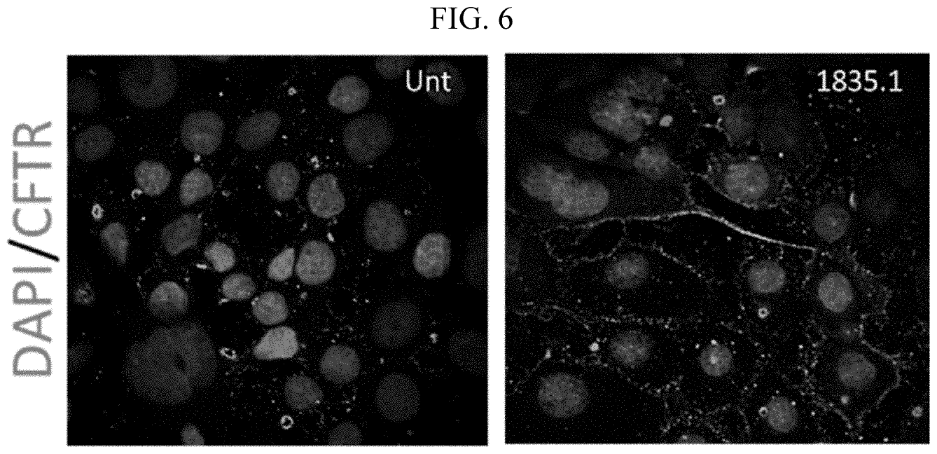

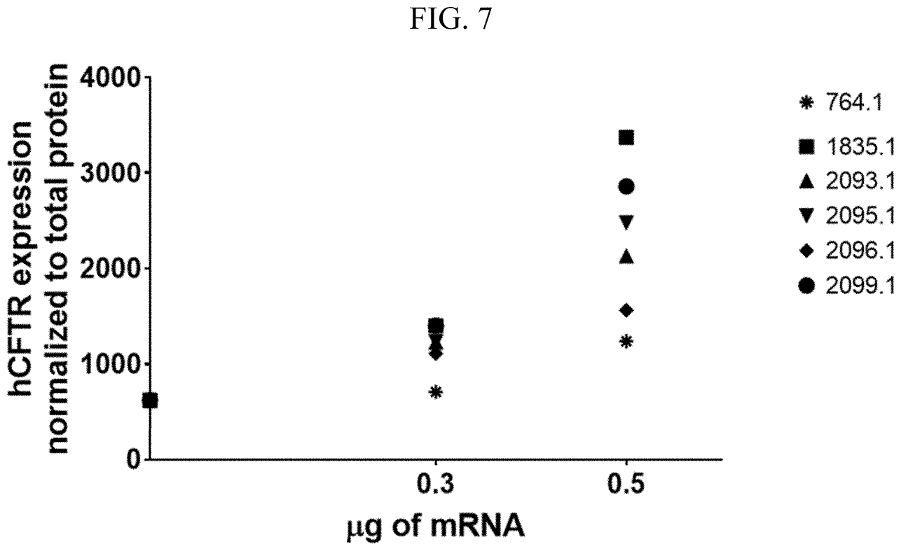

The expression of hCFTR for selected codon-optimized mRNA constructs was studied as a function of aliquot level. In this study, FRT (Fischer rat thyroid gland) cells were transfected with unformulated aliquots of 0 μg, 0.3 μg and 0.5 μg for each of the selected codon-optimized hCFTR mRNAs and the reference sequence (SEQ ID NO: 47, construct 764.1). The hCFTR constructs used in this study were 1835.1 (SEQ ID NO: 53), 2093.1 (SEQ ID NO: 66), 2095.1 (SEQ ID NO: 68), 2096.1 (SEQ ID NO: 69), and 2099.1 (SEQ ID NO: 72). After transfection, protein lysates were analyzed by WB as described in previous examples. C-band levels were analyzed and plotted for each of the constructs at each aliquot level. The results are presented in FIG. 7. At both the 0.3 μg and 0.5 μg levels, all codon-optimized constructs showed better expression than the reference sequence. It can also be seen that expression levels were relatively similar for the codon-optimized constructs the 0.3 μg level. However, at the 0.5 μg level, clear differences could be seen among the various codon-optimized constructs, and the constructs ranked differently based on their C-band protein expression levels, with SEQ ID NO: 53 being the highest C-band expressing construct, as shown in FIG. 7.

### Example 9: Transfection Efficiency in FRT Cells Transfected with mCherry mRNA

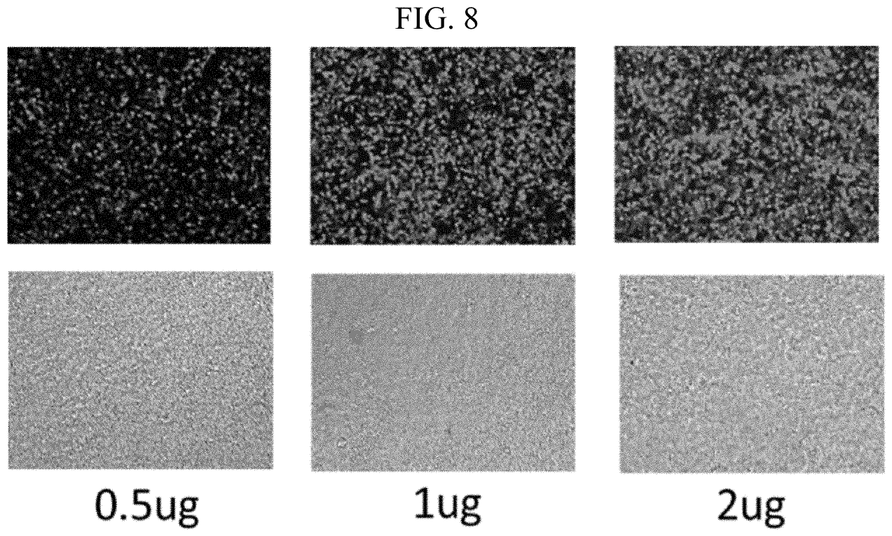

To further verify that FRT cells are effectively transfected by mRNAs in a dose-dependent manner, FRT cells were transfected with 0.5 μg, 1 μg, and 2 μg of mRNA expressing the mCherry monomeric red fluorescent protein. 6 hours post transfection, the transfected cells were imaged using confocal fluorescence microscopy. The results are shown in FIG. 8, with top panels showing the fluorescent images for transfected cells at each dose level and the bottom panels showing images for untransfected cells. The transfection efficiency was determined to be 80%, with a dose-dependent increase in mCherry expression as the 5 μg treated cells showed significantly greater fluorescence intensity than the 0.5 μg and 1 μg treated cells. Thus, this experiment confirms that FRT cells are effectively transfected with mRNAs in a dose-dependent manner.

### Example 10: Efficacy in FRT Cells Transfected with Selected hCFTR mRNAs

Further studies were conducted to assess the activity of hCFTR proteins expressed by selected mRNA constructs and whether these can properly open and close to allow proper ion transport. In these studies, an Air-Liquid Interface (ALI) cell culture model of FRT cells was used to transfect an unformulated subset of codon-optimized hCFTR mRNAs at different doses ranging from 0.5 μg-2 μg of mRNA (the 2 μg dose is not shown) into the FRT cells. ALI is a method of cell culture by which polarized cells are generated with their basal surfaces in contact with media, and the top of the cellular layer is exposed to the air. This model helps to mimic the cellular structure of in vivo airways.

At 24 hours post-transfection, transepithelial conductance (Gt) of the cells over time was measured as an indicator of CFTR activity. Initially, Gt was measured with the transfected or control cells unperturbed. Then, a sequential process of CFTR activation (channel opening), enhancement (gating promotion) and closing of the CFTR channels was performed. The hCFTR constructs used in this study were 1835.1 (SEQ ID NO: 53), 2093.1 (SEQ ID NO: 66), 2095.1 (SEQ ID NO: 68), 2096.1 (SEQ ID NO: 69), and 2099.1 (SEQ ID NO: 72). In addition, controls were performed using a reference sequence of construct 764.1 (SEQ ID NO: 47) and untransfected cells. In this process, the cells were first stimulated with Forskolin, a cAMP-dependent CFTR channel activator. Once an equilibrium was reached with the Forskolin, the potentiator VX770 was introduced to further promote gating. Finally, after a new equilibrium was reached with the VX770, Inh-172, a known inhibitor of the CFTR channels, was added. Further information on the protocols used in these measurements can be found in the literature (Schultz et al. (1999) Physiol. Rev., 79:S109-44; Li et al. (2004) J. Cyst. Fibros. Supple. 2:123-6.).

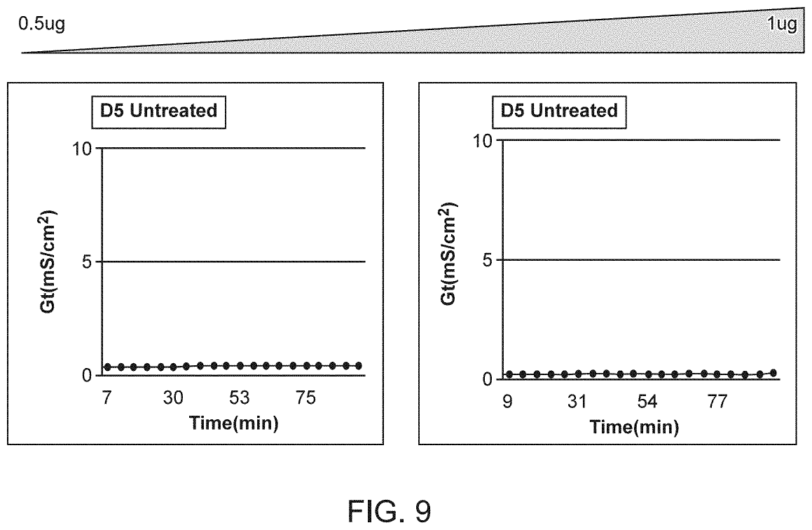

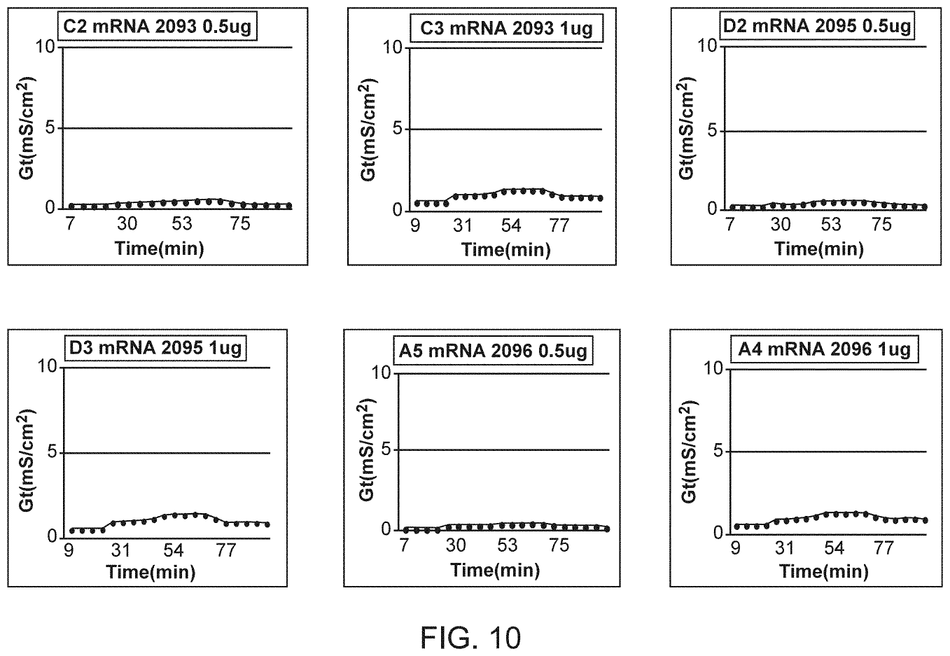

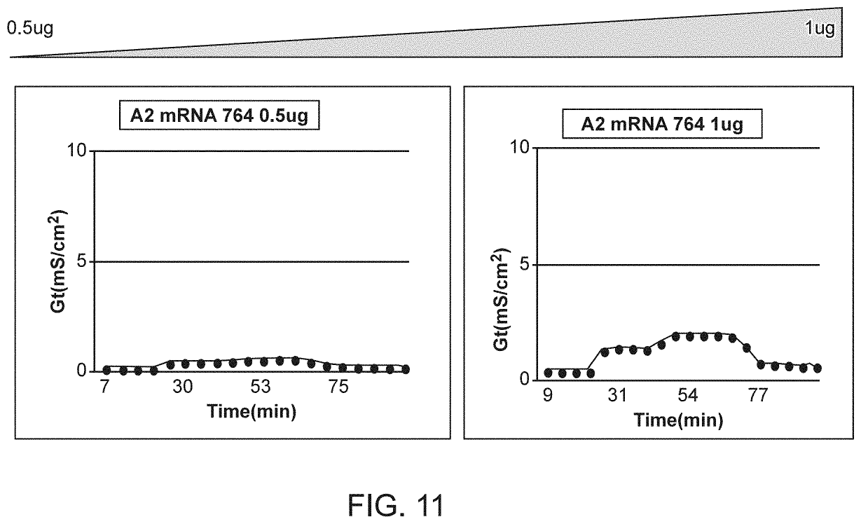

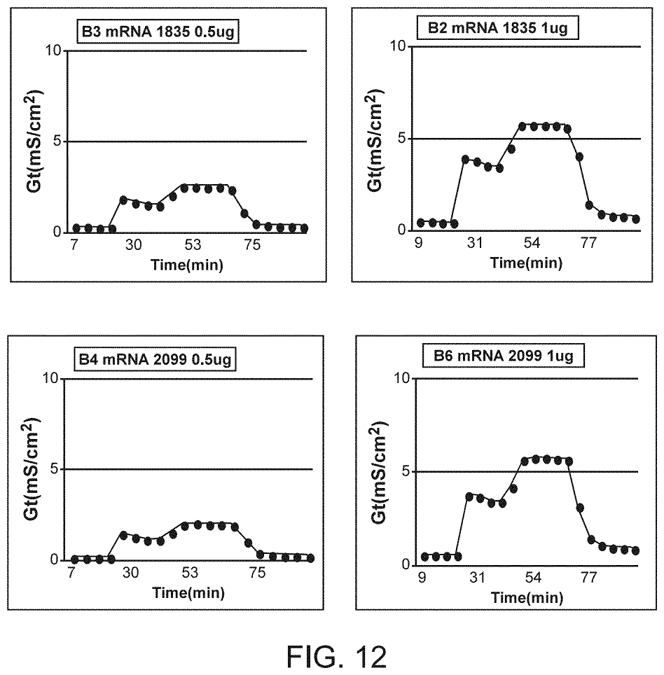

The results of these studies are shown in FIGS. 9 through 12. FIG. 9 shows the results for two untreated cells with Gt values being non-existent or near zero at all stages of the process. FIG. 10 shows the results for codon-optimized hCFTR mRNA constructs 2093, 2095, and 2096, which showed some Gt values upon activation (Low Gt responders), but still relatively low activity compared to the reference sequence values shown in FIG. 11 with a Gt value of about 2 for the 1 μg dose. Finally, FIG. 12 shows that the constructs 2099.1 (SEQ ID NO: 72) and 1835.1 (SEQ ID NO: 53) had a 3-fold increase in Gt (Gt of about 6) over the reference sequence (Good Gt responders). In addition, it can be observed in FIG. 12 that an initial increase in Gt was seen at a time point of about 25 to 30 minutes, which was associated with the introduction of Forskolin. A second increase in Gt was observed at about 40 minutes, which was associated with the introduction of VX770. Finally, a decline in Gt was observed at about 77 minutes, which was associated with the introduction of Inh-172. These inflection points further indicate that the CFTR proteins were highly active and responsive to conventional CFTR activators and inhibitors.

### Example 11: Minimal Immunostimulatory Activity of Lipid-Encapsulated mRNA

To determine the immunogenic effects, if any, of lipid-formulated hCFTR mRNA, several different lipid formulations were prepared with a mRNA construct of the disclosure. The lipid formulations included cholesterol and DSPC helper lipid and varied as to the ionizable cationic lipid, helper lipid, and PEG-lipid used in the formulation. Selected formulations, designated LF-3 (using Lipid #3, PEG550-PE, and DOTMA), LF-5 (using Lipid #3, PEG750-PE, and DOTMA), LF-7 (using Lipid #4), LF-8 (using Lipid #5), and LF-9 (using Lipid #3, DOTMA, and PEG2000-DMG) were used in this study. If not specified, the PEG-lipid was PEG2000-DMG.

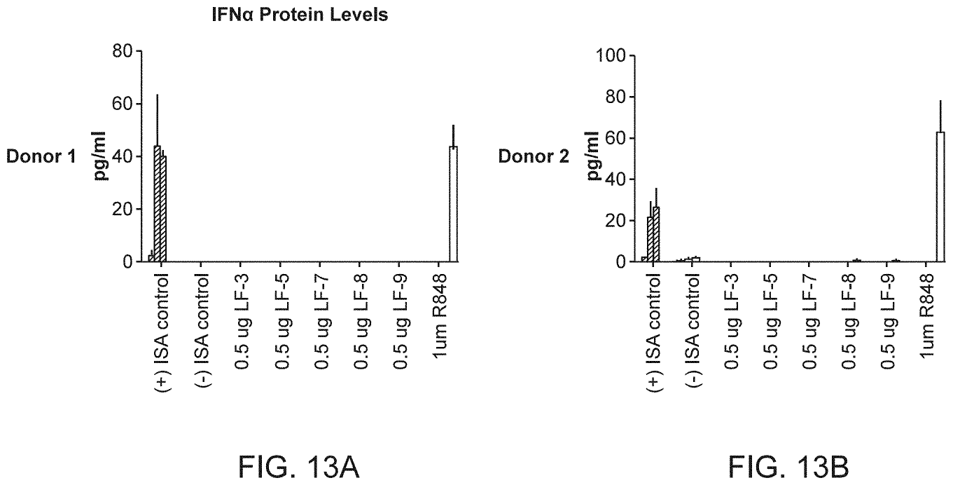

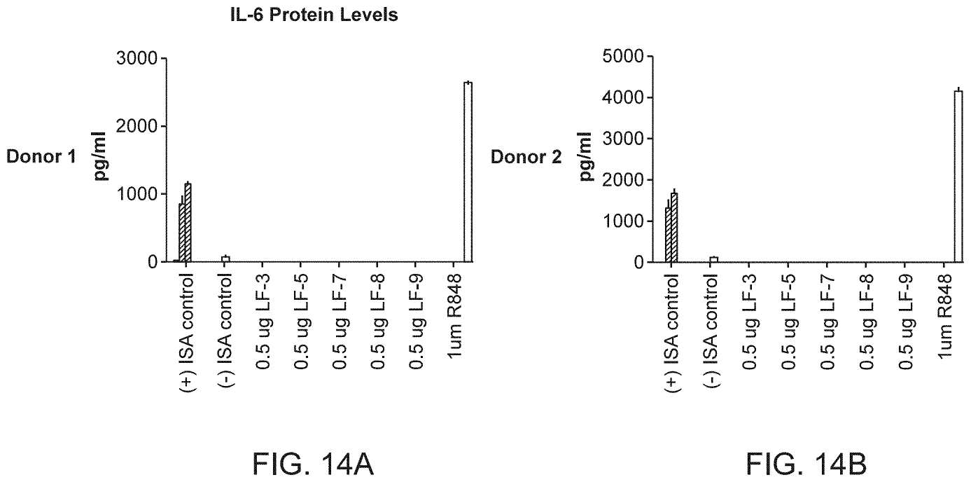

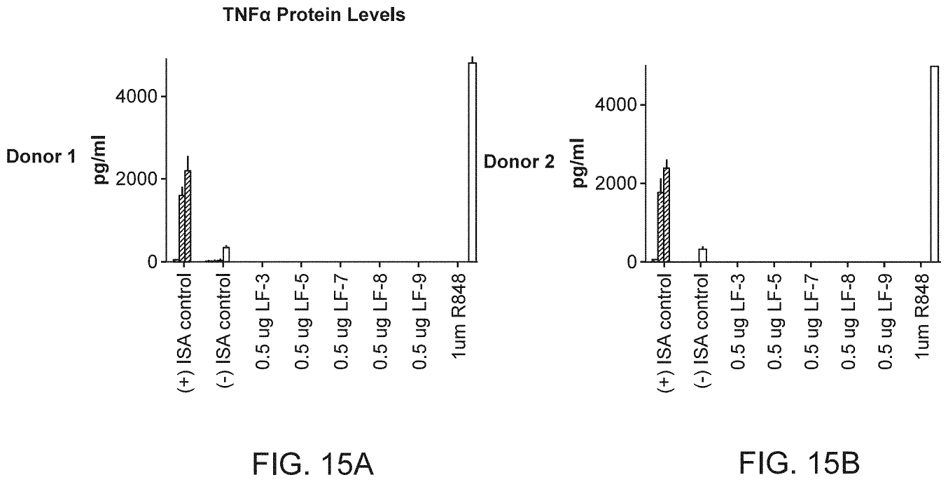

To test for immunogenic effects, fresh peripheral blood mononuclear cells (PBMCs) were isolated from two donors, and the cells were then treated with 0.5 μg of each lipid formulation and incubated at 37° C. In addition, a positive immunostimulatory assay ((+) ISA) control, a negative immunostimulatory assay ((−) ISA) control, and a comparative formulation of Resquimod (R-848), a drug that acts as an immune response activator, were also tested. After 24 hours of treatment, the cells were lysed and supernatants were collected and analyzed for cytokine expression levels. IFN-α measurements are shown in FIGS. 13A and 13B, IL-6 in FIGS. 14A and 14B, and TNF-α in FIGS. 15A and 15B. It can be seen that no detectable levels of IFN-α, IL-6 or TNF-α were observed in human PBMCs following treatment with lipid-formulated hCFTR mRNAs of the present disclosure. However, the (+) ISA and R-848 controls showed appreciable levels of IFN-α, IL-6 or TNF-α. These results indicate that the hCFTR mRNA-lipid formulations described herein have low immunogenicity.

### Example 12: Lipid Formulations Shield and Protect the mRNA in CF Sputum

To test the effectiveness of the hCFTR mRNA-lipid formulations of the present disclosure at encapsulating the mRNA and protecting it from degradation, several different lipid formulations were prepared with a mRNA construct of the disclosure. The lipid formulations varied as to the ionizable cationic lipid used in the formulation. Selected formulations, designated LF-1 (using Lipid #1), LF-2 (using Lipid #2), LF-3 (using Lipid #3, PEG550-PE in a lower concentration, and DOTMA), LF-4 (using Lipid #3, PEG550-PE in a higher concentration, and DOTMA), LF-5 (using Lipid #3, PEG750-PE, and DOTMA), LF-6 (using Lipid #3), LF-7 (using Lipid #4), LF-8 (using Lipid #5), and LF-9 (using Lipid #3, DOTMA, and PEG2000-DMG) were used in this study.

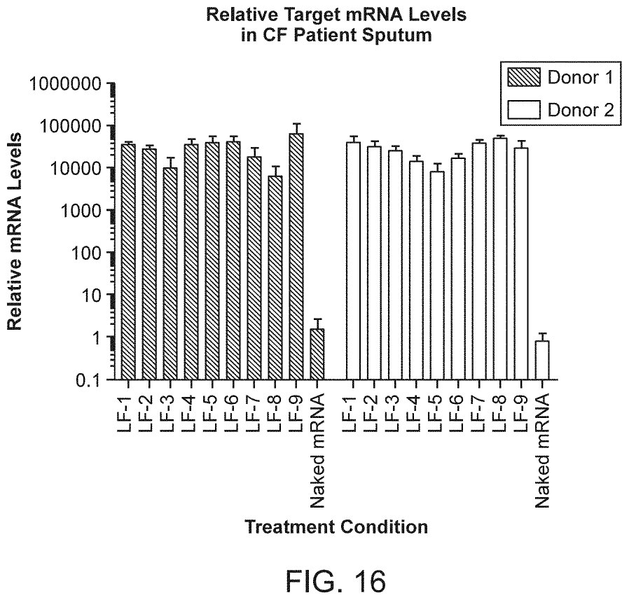

CF sputum from two donor patients were obtained. The hCFTR mRNA-lipid formulations were then tested by combining them with an aliquot of each sputum and incubating each sample for 24 hours. Unformulated mRNA (i.e., naked mRNA) was used as a control. Quantitative PCR (qPCR) was used to assess the relative mRNA levels. The results of this quantitation are shown in FIG. 16. As can be seen, all hCFTR mRNA-lipid formulations showed high relative mRNA levels while the unformulated mRNA showed significant degradation. Thus, the hCFTR mRNA-lipid formulations shield and protect the mRNA from degradation.

### Example 13: Lipid Formulations are Distributed in Upper and Lower Airways

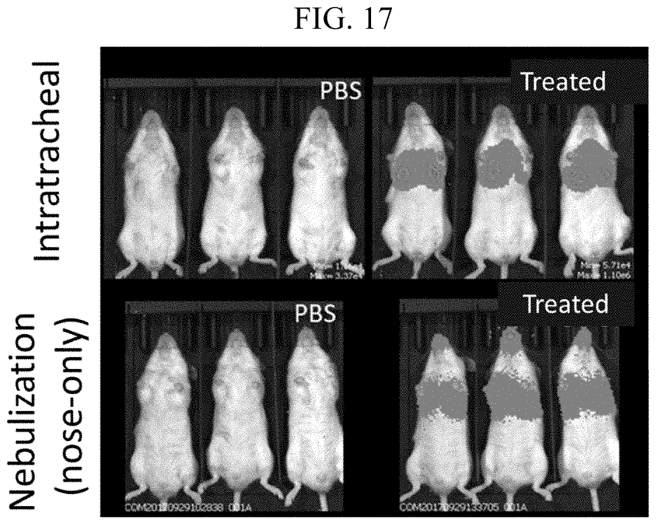

Further studies were conducted to assess efficacy of different administration routes on the expression of mRNA-lipid formulations. A nebulizable composition of a luciferase mRNA-lipid formulation prepared as described in Example 1 was developed by combining in a 1:1 volume ratio with water for injection (WFI). A dose of 0.1 mg of luciferase mRNA/kg was administered intratracheally via a bolus delivered by syringe and a dose of 0.2 mg of luciferase mRNA/kg was administered via nose-only nebulization in wild-type rats. After 6 hours, the rats were injected with luciferin to induce luminescence via luciferase catalysis and luminescence images were acquired using an in vivo imaging system (IVIS®, Perkin Elmer). Phosphate buffered saline (PBS) was used as a negative control for both administration routes. The acquired images are provided in FIG. 17. It can be seen that the intratracheally-treated group displayed in the top panel showed luminescence in the lung, whereas the nose-only nebulization group displayed in the bottom panel showed luminescence in both the nose and lung systems. The PBS controls did not show any luminescence. These results indicate that the mRNA-lipid formulations of the disclosure are able to effectively transfect both nose and lung-tissue systems, which represent the upper and lower airways.

### Example 14: Lipid Formulations Delivered a Reporter mRNA into Wild-Type Murine Lung Epithelial Airways

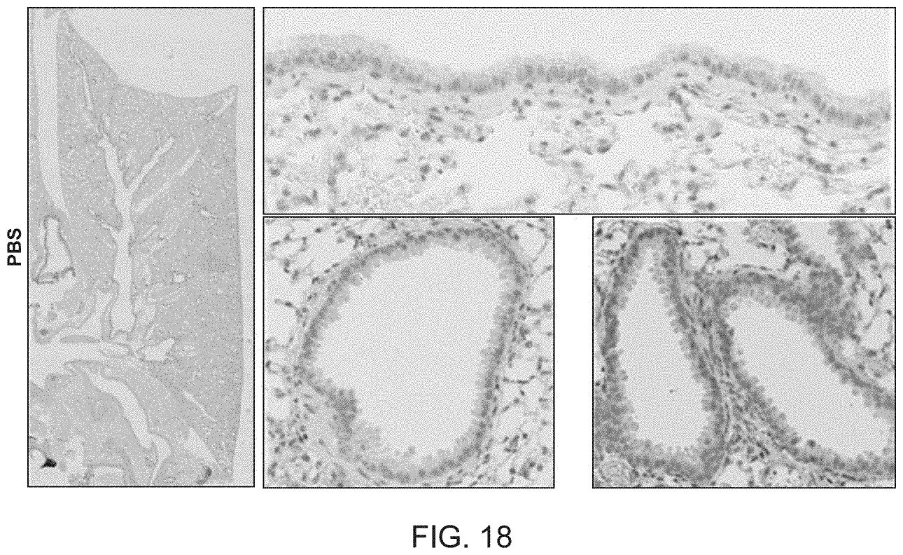

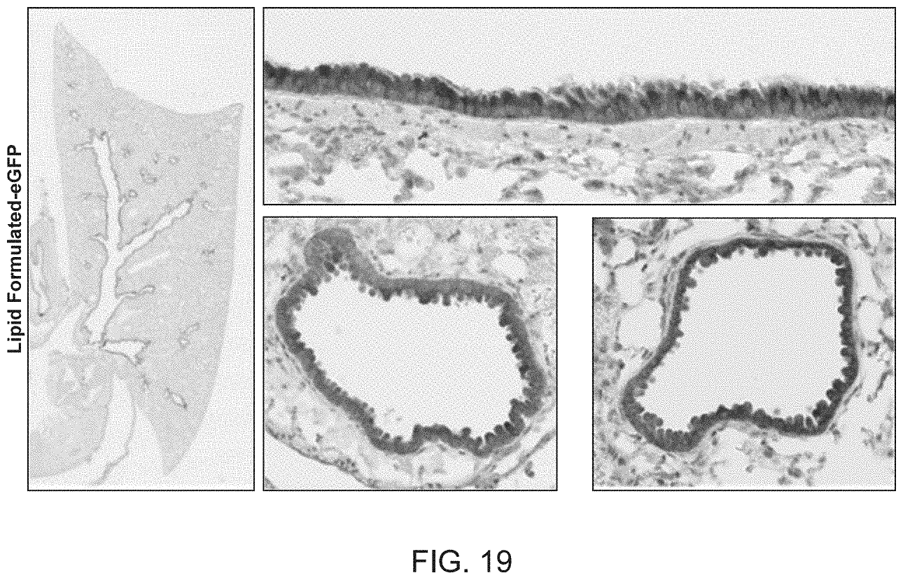

To determine whether lipid-formulated mRNAs delivered to the airways effectively transfect lung epithelial cells in vivo, an enhanced green fluorescent protein (eGFP) reporter mRNA was formulated into a lipid formulation according to the method described in Example 1. Wild-type mice were dosed intratracheally via a bolus delivered by syringe with 0.4 mg/kg of an optimized eGFP mRNA-lipid formulation and a negative control of PBS. The mice were then euthanized 24 hours later, and their lungs were extracted and processed for histology. The lung samples were treated with an eGFP-specific antibody and confocal fluorescence microscopy images were taken. FIG. 18 shows images for the negative PBS controls. It can be seen that no fluorescence was detected throughout the tissue. In contrast, images for the eGFP mRNA-treated mice shown in FIG. 19 displayed immunostaining in both the large and small airways. Thus, the mRNA-lipid formulations effectively transfected lung epithelial cells.

### Example 15: Lipid Formulations Efficiently Deliver the Cargo mRNA in the Epithelial Airways of a Transgenic Mouse Model

To further test whether the mRNA-lipid formulations can efficiently deliver mRNA to lung epithelial cells, a TdTomato fluorescent experiment was designed and conducted. In this experiment, transgenic floxed TdTomato mice were used. These mice were engineered to have a gene encoding TdTomato fluorescent reporter protein that also includes a CRE-based stop cassette (i.e., floxed cassette), which prevents complete transcription of the TdTomato gene in the absence of CRE recombinase (CRE). The floxed TdTomato mice are deficient in the CRE gene.

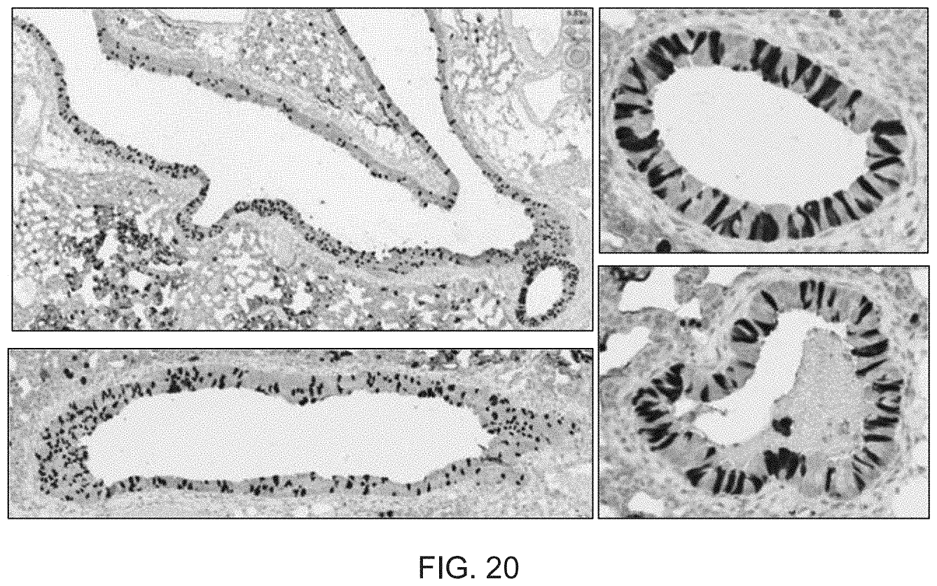

The floxed TdTomato mice were dosed intratracheally at 1 mg/kg with an optimized CRE mRNA-lipid formulation prepared according to the method described in Example 1. The mice were euthanized 72 hours later to allow full recombination of the floxed cassette by the CRE protein. Then, the lungs were extracted and processed for immunohistochemistry. The lung samples were treated with a TdTomato-specific antibody, and confocal immunofluorescence microscopy was used to collect images of the samples. FIG. 20 shows the image for mice treated with CRE mRNA-lipid formulations, which were able to generate a CRE protein that excised out the floxed cassette, allowing the expression of the TdTomato protein. TdTomato immunostaining was present in epithelial cells throughout large and small airways, thus indicating that the CRE mRNA-lipid formulations efficiently delivered the mRNA cargo to lung epithelial cells of both the large and small airways.

### Example 16: Lipid Formulations Target Ciliated Epithelial Cells

A further experiment was designed and conducted to test whether the mRNA-lipid formulations were specifically targeting only lung epithelial cells. In this experiment, the Floxed-TdTomato transgenic mice approach described in Example 15 was used, and the main cellular populations expressing the TdTomato protein were profiled. Briefly, 1 mg/kg of mRNA-lipid formulation was delivered intratracheally to airways of Cre/LoxP mice. Upon Cre recombination, cells express the TdTomato protein that can be visualized by immunohistochemistry using an anti-TdTomato antibody. Co-localization of TdTomato with FoxJ1, a marker for ciliated epithelial cells, was analyzed by staining samples with an anti-FoxJ1 antibody. DAPI was used as a general counterstain for cellular nuclei to show all cells, including cells that were not ciliated and did not express the CRE protein.

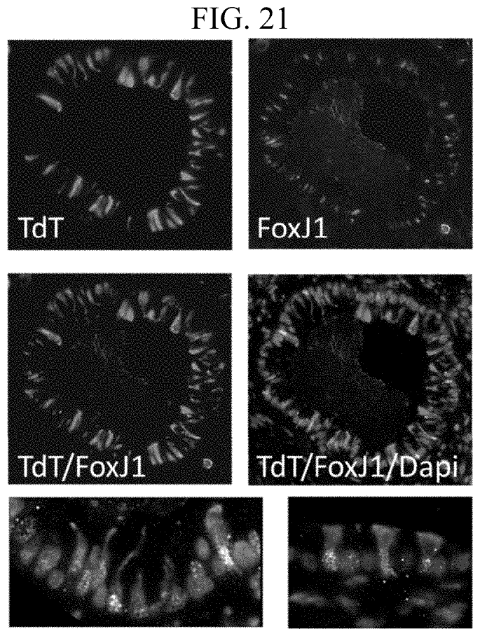

Samples were treated with a specific FoxJ1 antibody, and fluorescence imaging was conducted. The captured images are shown in FIG. 21. The first image labeled TdT is a sample that was not treated with FoxJ1 or DAPI, but represents a lung sample from floxed TdTomato mice treated with a CRE mRNA-lipid formulation. It can be seen that this image shows fluorescence only at the epithelial layer. The second image, labeled FoxJ1, represents a sample treated with only the FoxJ1 stain and processed for immunofluorescence. This images specifically highlights ciliated epithelial cells. The third image, labeled TdT/FoxJ1, represents a lung sample taken from floxed TdTomato mice treated with CRE mRNA-lipid formulation and stained with anti-FoxJ1 antibody. It can be seen that the fluorescence due to TdTomato and FoxJ1 are colocalized at the lung epithelium, thus confirming that TdTomato was indeed associated with lung epithelial cells. Finally, the fourth image, labeled TdT/FoxJ1/DAPI, represents a lung sample taken from floxed TdTomato mice treated with CRE mRNA-lipid formulation, followed by sample staining with anti-FoxJ1 antibody and DAPI. This image shows the colocalization of TdTomato and FoxJ1; however cells peripheral to the FoxJ1/TdTomato-positive cells only stained with DAPI. This indicates that the CRE mRNA-lipid formulation specifically targeted the lung epithelial cells, and did not transfect deeper layers of cells. Further images of TdT/FoxJ1 colocalization at high magnification are shown in the bottom panel in FIG. 21.

These results show that lipid-formulated mRNA is efficiently delivered to ciliated epithelial cells in rodents.

### Example 17: Cellular Profiling of the Nasal Epithelia Indicates that Lipid Formulations are Taken Up by Ciliated Epithelial Cells

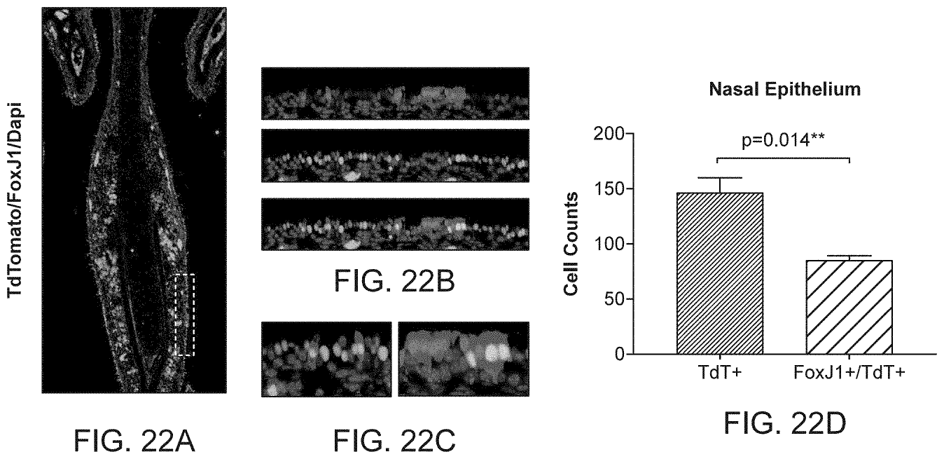

To test uptake in nasal epithelial cells, the floxed-TdTomato mice protocol described in Examples 16 was also used to conduct co-localization experiments with TdTomato and FoxJ1 in mice treated with different CRE mRNA-lipid formulations. The formulations were delivered intranasally by droplet deposition. After 72 hours, the mice were euthanized, and the nasal portion of the head underwent a decalcification process to remove the bone but keep the structure of the nasal epithelia intact. When completed, the nasal epithelial tissue samples were processed for immunofluorescence following the procedures described in Example 16. In these experiments, TdTomato fluorescence is indicative of cells targeted by the CRE mRNA-lipid formulations, and FoxJ1 is indicative of ciliated cells in the nasal epithelia. DAPI was used as a counterstain of cellular nuclei to show cells that were not ciliated and not transfected with CRE mRNA. The resulting confocal fluorescence microscopy images are shown in FIG. 22. The images shown in Panel A provide a panoramic view of the nasal septa. Panels B and C provide high magnification images of the area indicated by the dashed rectangle in Panel A. In agreement with the lung studies of Example 16, colocalization of FoxJ1 and TdTomato was observed, indicating successful transfection of ciliated epithelial cells in the nasal epithelium, while only DAPI was observed in other cells. Panel D provides a quantitative plot of cell counts for all cells expressing TdTomato (TdT+) as well as cells expressing both TdTomato and FoxJ1 (FoxJ1+/TdT+). The results indicate that 60% of the cells that took up lipid-formulated CRE mRNA were ciliated cells. Thus, CRE mRNA-lipid formulations showed high selectivity toward ciliated epithelial cells of the nasal epithelia.

### Example 18: Different Lipid Formulations can Efficiently Target the Murine Epithelial Airways

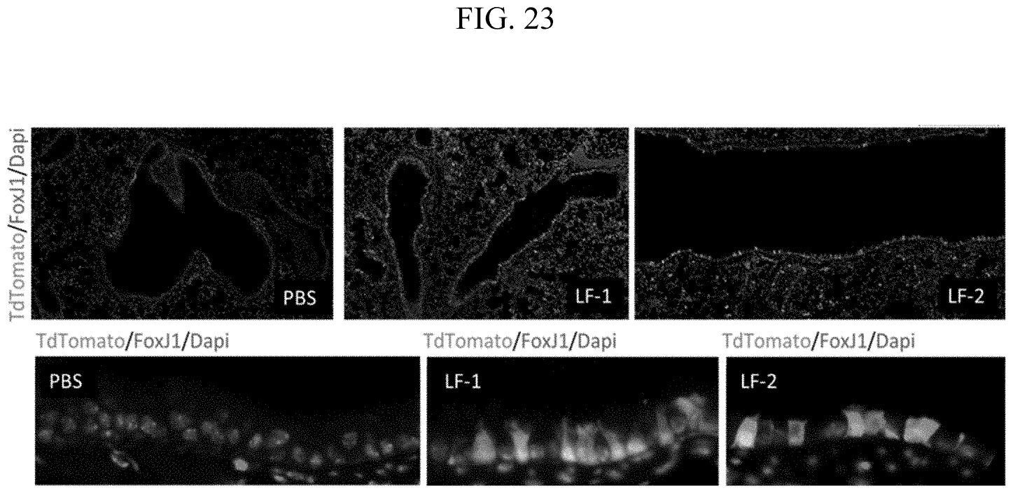

The floxed-TdTomato mice experiments described in Example 16 were repeated to test co-localization of TdTomato and FoxJ1 in mice treated with different CRE-mRNA-lipid formulations (LF-1 and LF-2 as described in Example 12). A further negative control of PBS was also used. The results are shown in FIG. 23, which shows that both the LF-1 and LF-2 formulations were able to express the CRE protein, thereby allowing expression of TdTomato, which co-localized with the FoxJ1 marker. The PBS-treated samples did not show any fluorescence. Thus, the different formulations both resulted in highly specific expression in the lung epithelial cells.

### Example 19: mRNA Kinetics in Mice Treated Intratracheally with hCFTR mRNA-Lipid Formulations

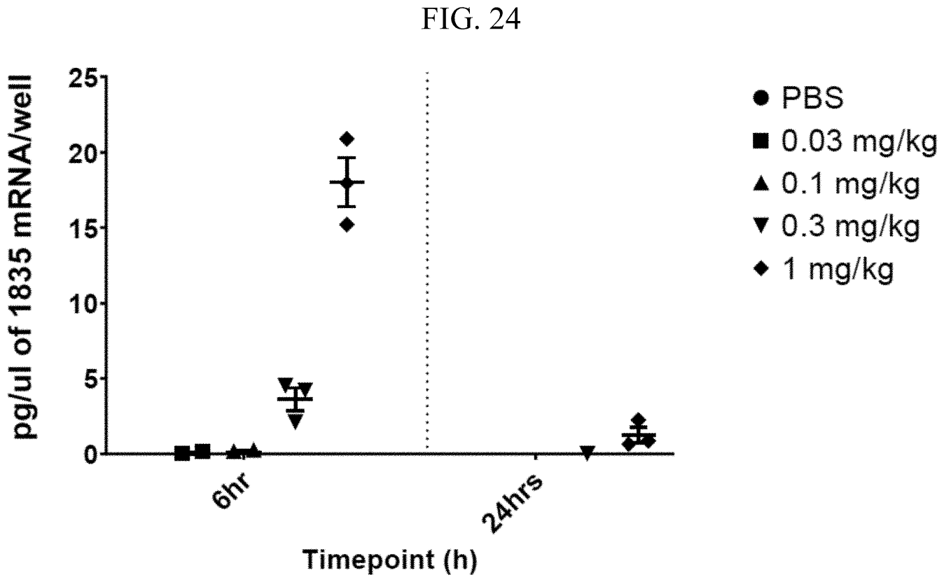

To study the rate of mRNA expression for hCFTR mRNA-lipid formulations, a formulation was prepared as described in Example 1 for in vivo monitoring experiments, using the 1835.1 construct (SEQ ID NO: 53). In these experiments, CFTR knockout (KO) mice (i.e., mice deficient in the CFTR gene) were dosed intratracheally via a bolus delivered by syringe at dose levels of 0.03, 0.1, 0.3 and 1 mg/kg of the hCFTR mRNA-lipid formulation. Additional mice were treated with the negative control of PBS. The mice were then euthanized at 6 hours or 24 hours, their lungs were extracted, and mRNA levels were quantified using the Quantigene® Assay. The results of the assay are shown in FIG. 24. At the 6-hour timepoint, increasing levels of mRNA were seen for increasing doses, but baseline levels of mRNA were reached by 24 hours. Thus, almost all of the mRNA was consumed within 24 hours of administration.

### Example 20: hCFTR Protein Levels are Detected in Mice Using a Protein Enrichment Protocol

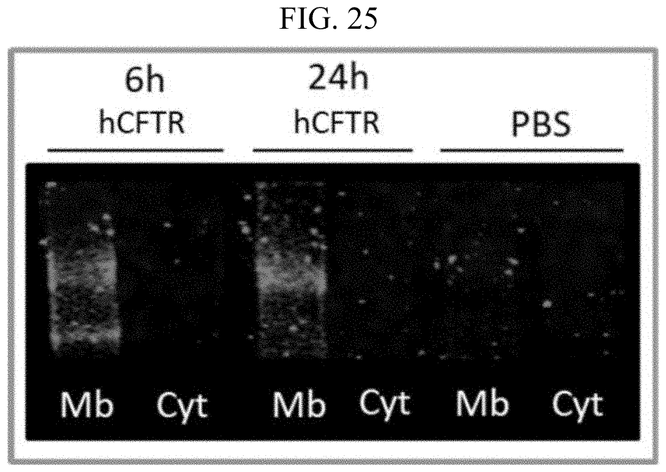

To further study the degree of protein expression of hCFTR mRNA-lipid formulations, samples derived from the experiments of Example 19 were first fractionated using standard protocols, and membrane and cytosolic fractions were generated. To characterize the hCFTR protein in membrane and cytosolic fractions, the fractions were analyzed by WB using an antibody specific for hCFTR according to the protocol described in Example 1. The results for the WB assays can be seen in FIG. 25. It can be seen that for the mice euthanized at the 6-hour timepoint, both C- and B-bands were observed at 170 kDa and 150 kDa, respectively. However, at 24 hours, only the C-band was observed at the expected size of 170 kDa. In addition, hCFTR bands were only observed in the enriched membrane fractions (labeled as Mb) and were not present in the cytosolic fractions (labeled as Cyt) or for the negative control of PBS. These results are in agreement with those of Example 18, showing that at 6 hours, mRNA expression was still ongoing in the cell, while by 24 hours, expression had been completed as seen by the presence of only the C-band corresponding to fully mature, glycosylated hCFTR, but not the B-band. In addition, these results indicate that hCFTR was expressed and properly localized in the cellular membrane.

### Example 21: mRNA Kinetics in Aerosolized hCFTR mRNA-Lipid Formulation

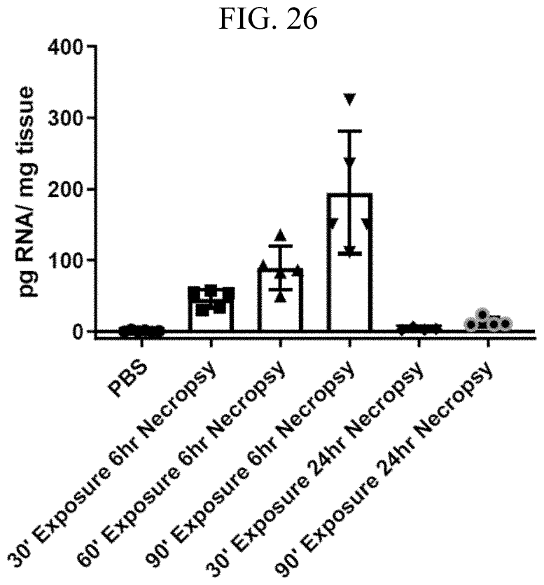

The effect of exposure time on hCFTR mRNA expression was also studied. A formulation was prepared as described in Example 1 for in vivo monitoring experiments using the 1835.1 construct (SEQ ID NO: 53), and wild-type rats were treated using a nose-only nebulization system. The rats were exposed to the formulation for 30, 60 or 90 minutes. The rats were then euthanized at either 6 hours or 24 hours post-exposure. Rat lungs were extracted and hCFTR mRNA levels were quantified by Quantigene® Assay. The results are shown in FIG. 26. It can be seen that an increase in exposure time correlated with hCFTR mRNA levels at the 6-hour time point, but by 24 hours post exposure, the mRNA levels reached a baseline level similar to the negative control of PBS. Thus, regardless of exposure time, the mRNA was completely consumed by 24 hours, while increased exposure duration resulted in increased mRNA uptake.

### Example 22: Analysis of Nasal Epithelium Samples of CFTR KO Mice Treated with hCFTR mRNA-Lipid Formulation

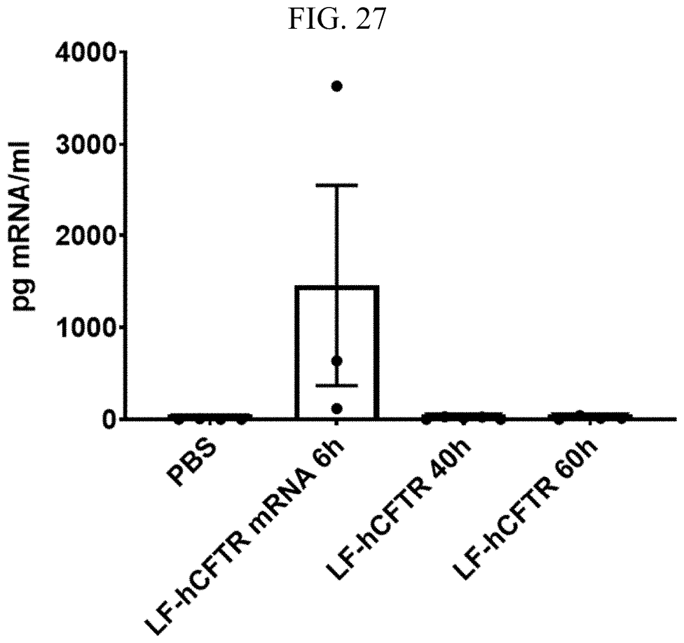

Extended time-based studies were conducted to further assess the kinetics of hCFTR mRNA delivery in vivo. CFTR KO mice were treated intranasally via a bolus delivered by syringe with a hCFTR mRNA-lipid formulation prepared as described in Example 1 using the 1835.1 construct (SEQ ID NO: 53, formulation LF-1). The mice were treated for two consecutive days with either the lipid formulation or a negative PBS control, receiving 50% of the daily dose in the morning and 50% of the daily dose in the afternoon as the mice could not internalize a full dose volume in a single administration. The mice were euthanized at either 6 hours, 40 hours, or 60 hours after the last dose, and nasal epithelium was extracted and analyzed for hCFTR mRNA content using the Quantigene® assay. The results are shown in FIG. 27. At 6 hours, mRNA levels peaked, and then mRNA levels reached baseline levels at 40-60 hours. These results indicate kinetics of mRNA consistent with consumption of mRNA by 40 hours after the last dose even after multiple doses and extended treatment regimens.

In addition, live mice were evaluated for hCFTR activity at 40 hours and 60 hours after the last dose. Specifically, the chloride channel current was measured by Nasal Potential Difference (NPD) according to standard protocols (Hodges et al., Genesis 46, 546-552, 2008).

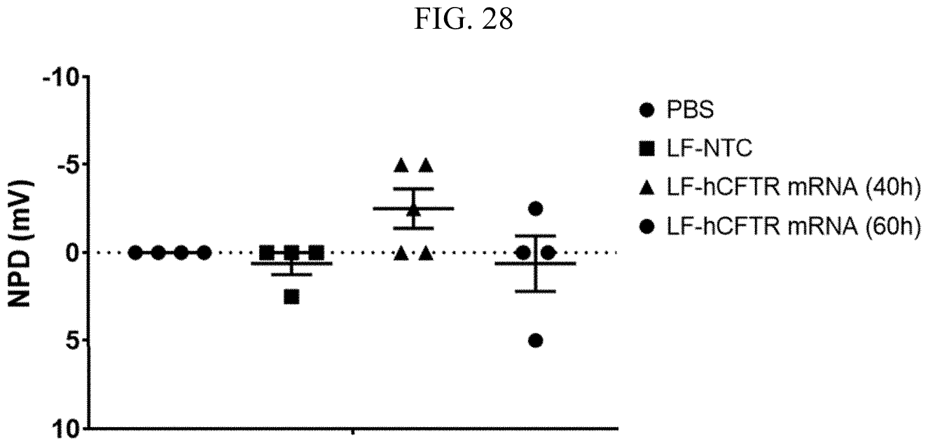

PBS and a non-targeting control (NTC) lipid formulation were used as negative controls in these experiments. The results of the NPD assay are shown in FIG. 28. At 40 hours, 3/5 of the mice showed increased current measurements and 1/4 showed functional activity at 60 hours. These results were consistent with the variability of the NPD assay. In contrast, the negative controls did not show any appreciable activity. These data indicate that the codon-optimized hCFTR mRNAs tested expressed functionally active hCFTR protein in vivo.

### Example 23: Nasal Potential Difference Measurements in CFTR KO Mice Treated with Different hCFTR mRNA-Lipid Formulations

The experiments of Example 22 were extended to test different hCFTR mRNA-lipid formulations. In this experiment, CFTR KO mice were treated intranasally with the hCFTR mRNA-lipid formulations for two consecutive days as described in Example 22. The specific hCFTR mRNA-lipid formulations used were prepared as described in Example 1, using construct numbers 1835.1 (SEQ ID NO: 53), 2099.1 (SEQ ID NO: 72), and the reference sequence construct number 764.1 (SEQ ID NO: 47). Additionally, PBS was used as a negative control. At 40 hours after the last dose, the chloride channel current was measured by NPD according to standard protocols (Hodges et al., Genesis 46, 546-552, 2008).

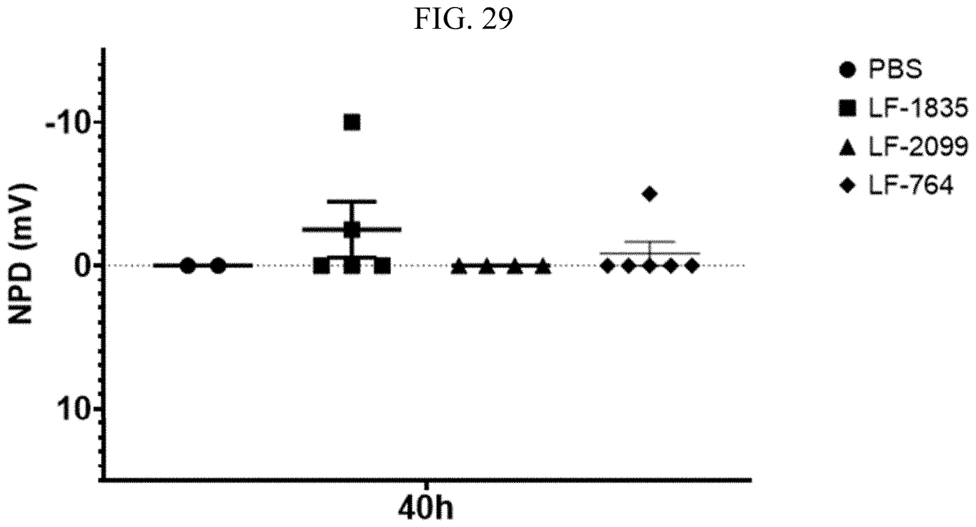

The results of the NPD assay are provided in FIG. 29. At 40 hours, the lipid formulation that included a construct of SEQ ID NO: 53 showed increased current in 2/5 of the mice. No current was observed for the lipid formulation that included a construct of SEQ ID NO: 72, and 1/6 of the mice were observed to have an increased current with the lipid formulation that included a construct of SEQ ID NO: 47. These results were consistent with the variability of the NPD assay. These data confirmed that the hCFTR mRNA having a sequence of SEQ ID NO: 53 expressed functionally active hCFTR protein in vivo and showed superior activity as compared to the negative control and the reference sequence.

### Example 24: Aerosolized Lipid Particles Generate a Breathable Droplet Size

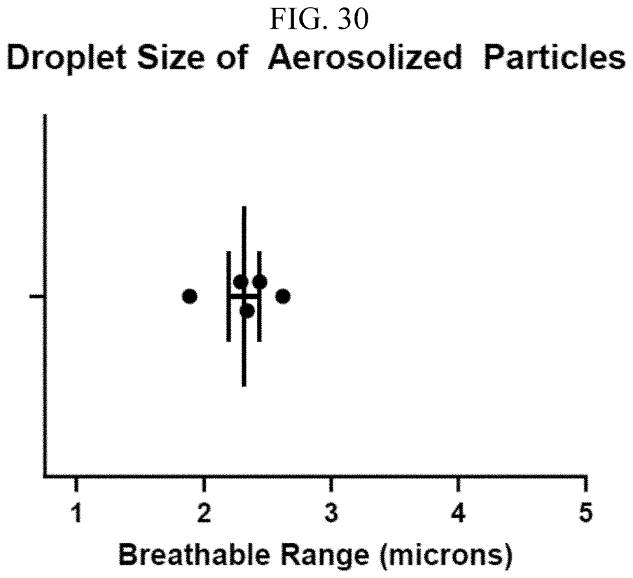

The mRNA-lipid formulations were further studied to determine whether they could be further developed to have acceptable properties for administration by inhalation. Typically, droplet particles that are less than 5 microns in diameter are considered to be highly breathable (Part. Fibre Toxicol. 2013; 10:12). An mRNA-lipid formulation was prepared for nebulization by diluting with WFI at a 1:1 volume ratio, and the aerosolized composition was analyzed by a cascade impactor. The results are shown in FIG. 30. It can be seen that the droplet size was consistently in the range of 2.3-2.5 microns in all the samples analyzed. This droplet size range indicates that the lipid particles are highly breathable for lung delivery.

### Example 25: Encapsulation of mRNA is Maintained Before and After Nebulization

Further analysis was conducted to ensure that lipid-formulated mRNA remained encapsulated both before and after nebulization. Six formulation lots of hCFTR mRNA-lipid formulation prepared as described in Example 1 were further prepared for nebulization by diluting with WFI at a 1:1 volume ratio. The nebulizable compositions were then analyzed by the RiboGreen fluorescent assay (Thermofisher Scientific) prior to nebulization to determine the initial percent encapsulation and percent yield of mRNA. The samples were then analyzed for percent encapsulation and percent yield of mRNA after nebulization by RiboGreen assay. RiboGreen is a fluorescent dye that is used in the detection and quantification of nucleic acids, including mRNA. In its free form, RiboGreen by itself exhibits little fluorescence and possesses a negligible absorbance signature. When bound to nucleic acids, the dye fluoresces with an intensity that is several orders of magnitude greater than the unbound form. The fluorescence can be detected by a sensor and the nucleic acid can be quantified.

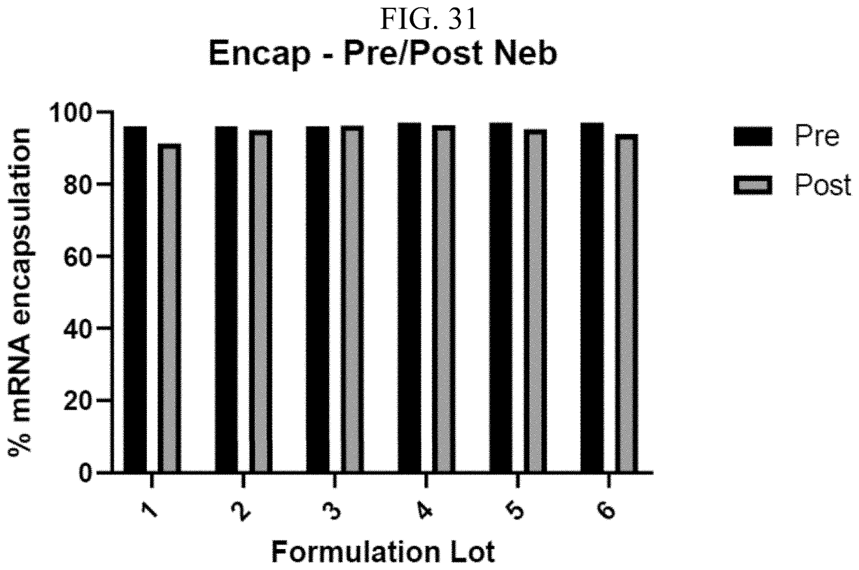

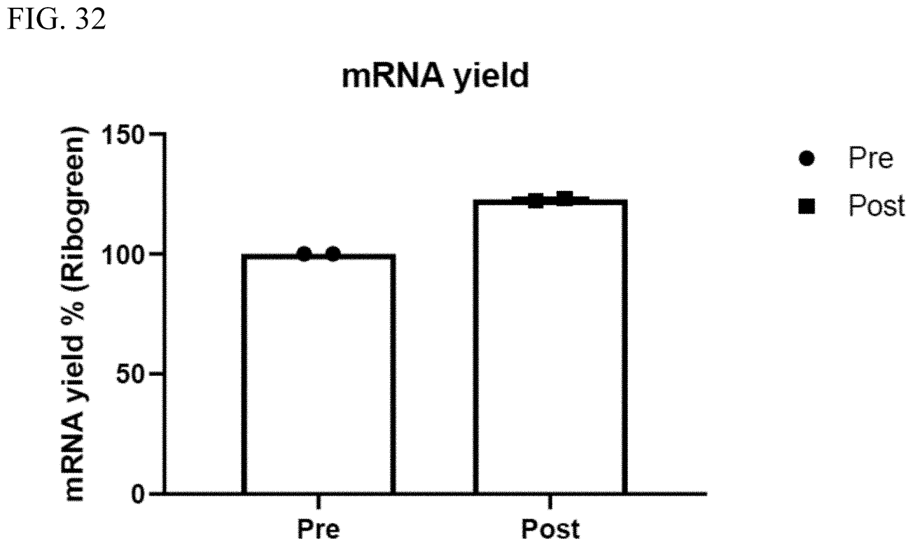

The results for percent encapsulation analysis are shown in FIG. 31. It can be seen that lipid particle integrity was maintained above at least about 90% both pre- and post-nebulization for all formulation lots tested. Thus, the lipid formulations described herein show good integrity. In addition, the results for average mRNA yield percent are shown in FIG. 32 and indicate that lipid-formulated mRNA exhibited a highly efficient recovery post-nebulization. Thus, the lipid formulations adequately encapsulate and protect the mRNA.

### Example 26: Integrity of mRNA Pre- and Post-Nebulization is Maintained

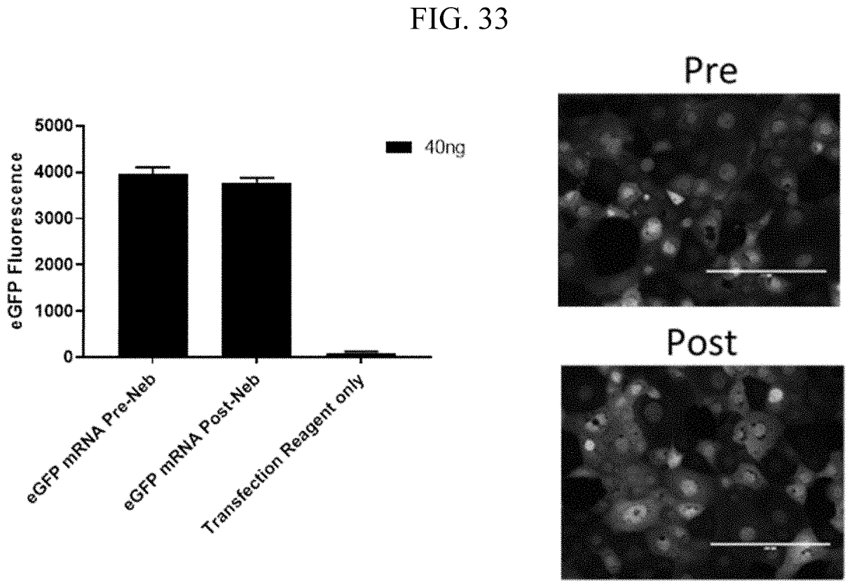

The ability of lipid-formulated mRNAs to transfect cells before and after nebulization was also studied. eGFP mRNA-formulations were prepared as described in Example 14 and prepared for nebulization by diluting with WFI at a 1:1 volume ratio. The nebulizable composition was aerosolized using a vibrating mesh nebulizer, which operates by vibrating many laser drilled holes at a high rate over a short distance creating a pump that draws medication through the holes and forming an incredibly small particulate mist. Pre- and post-nebulization fractions were collected, and the mRNA was extracted from the lipid formulations in both fractions. Then, the unencapsulated eGFP mRNA from each fraction was used to transfect CFBE cells, the cells were treated with an eGFP-specific antibody, and confocal fluorescence microscopy images were taken 6 hours post-transfection. The transfection reagent used was Lipofectamine 3000 (Invitrogen). Fluorescence levels were also quantified. The images shown in the right panel of FIG. 33 displayed high fluorescence in the cells both before and after nebulization, which indicates that both fractions successfully transfected the CFBE cells. Likewise, the quantitative measurements (background corrected and normalized to cell number) graphed in the left panel of FIG. 33 showed a similar degree of fluorescence for both fractions, while the negative control of transfection reagent showed no appreciable fluorescence. These results thus indicate that mRNA integrity was maintained throughout the nebulization process.

### Example 27: Dose-Dependent Integrity of mRNA Pre- and Post-Nebulization is Maintained

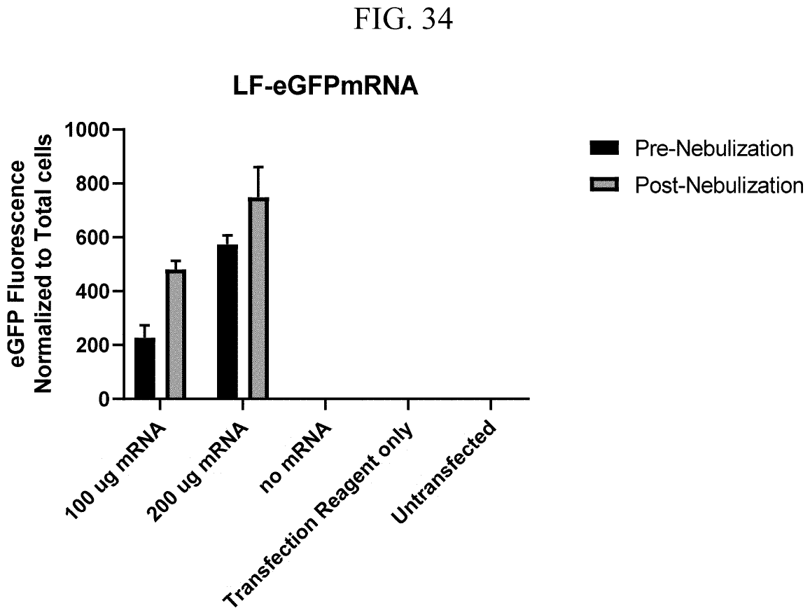

The experiments described in Example 26 were repeated in a dose-dependent manner to test whether mRNA integrity was maintained at higher doses. Pre- and post-nebulization fractions were collected and the lipid formulated-eGFP mRNA formulations from these fractions were used to transduce CFBE cells at two different doses (100 and 200 μg). Then, eGFP fluorescence levels were quantified for both fractions and transfection doses along with experiments for the negative controls of no mRNA (empty lipid particle), transfection reagent only (Lipofectamine 3000), and untransfected cells. A graph of the results is provided in FIG. 34, which shows a dose-dependent increase in fluorescence (normalized to total cells) for the lipid formulated eGFP mRNA, but no appreciable fluorescence for the negative controls. This dose-dependent increase in fluorescence indicates that kinetics and mRNA integrity were preserved throughout the nebulization process, even at higher doses.

### Example 28: Ex Vivo Lung Explants—Non-CF Human Lungs

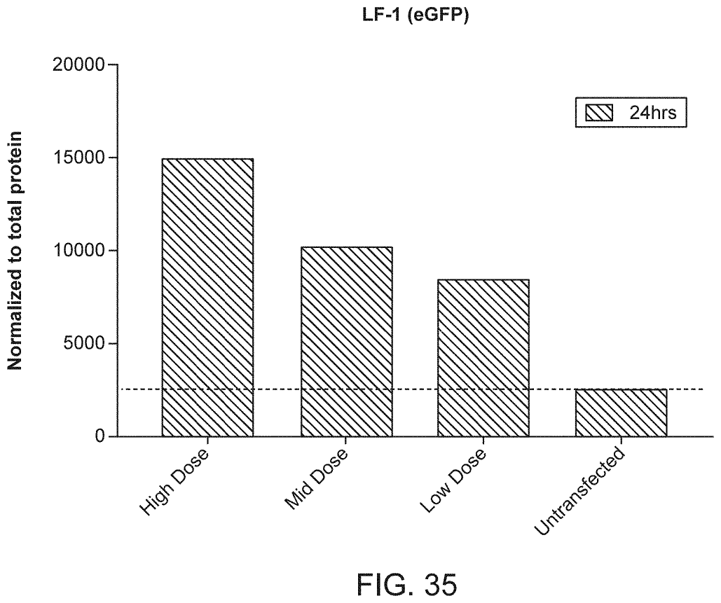

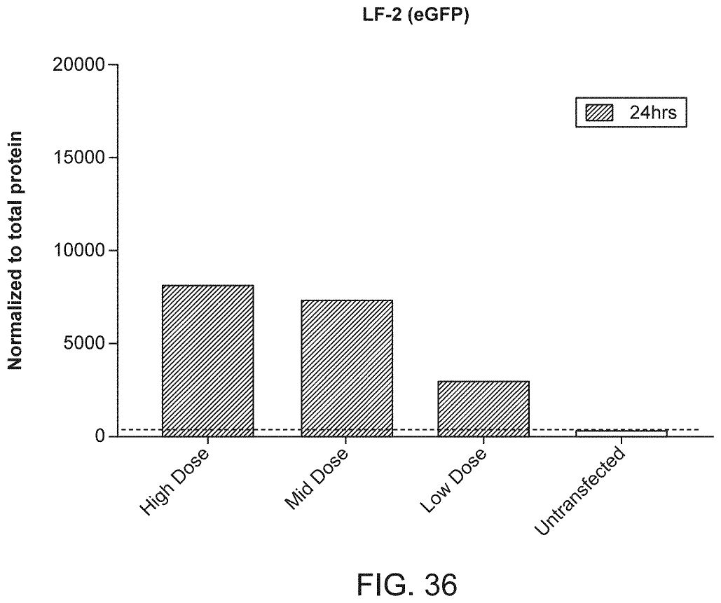

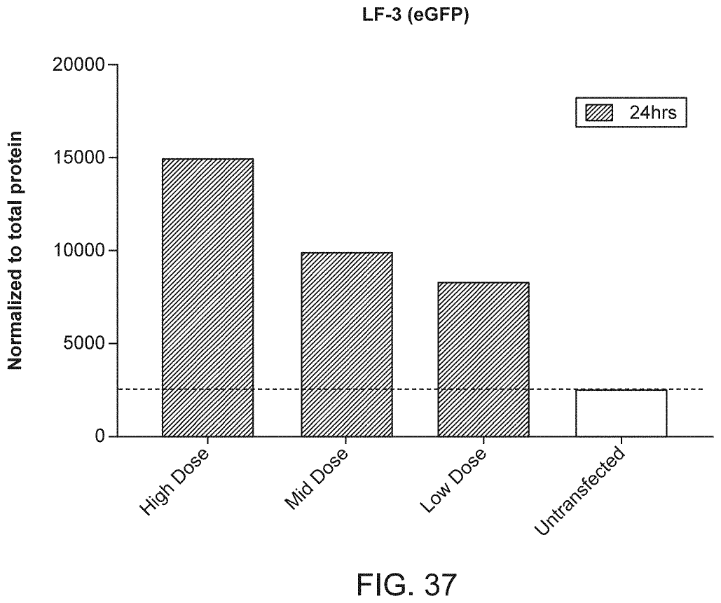

The mRNA-lipid formulations were further tested for their ability to effectively express protein in human lungs. Per the protocols described in Example 2, an extracted set of human lungs from a non-CF subject was received and insufflated with low-melting temperature agarose. A conical piece was excised and 250-micron slices were generated using a slice microtome. The slices were incubated, and cell culture medium was changed several times to remove the excess of agarose. The slices were then incubated with different eGFP mRNA-lipid formulations at three different dose levels (low, mid, and high). The lipid formulations used were LF-1 (low lipid to mRNA weight ratio), LF-2 (mid lipid to mRNA weight ratio), LF-3 (high lipid to mRNA weight ratio), which differed in composition from those used in other examples. The lipid portion of these formulations was identical and included Lipid #3, DOTAP, DSPC, cholesterol, and PEG2000-DMG in the same ratios. In addition, a sample of untransfected lung extract was tested as a negative control. Cell viability was monitored through the entire incubation process and was maintained for all the formulations and doses analyzed. 24 hours post incubation, samples were processed for WB and analyzed for eGFP expression. The eGFP band was quantified (normalized to total protein) and plotted as shown in FIGS. 35 (LF-1), 36 (LF-2), and 37 (LF-3). All the formulations analyzed showed a dose-dependent increase in expression levels, indicating that the lipid formulations effectively transduced expression of mRNA in a human lung matrix.

### Example 29: Ex Vivo Lung Explants—CF Human Lungs

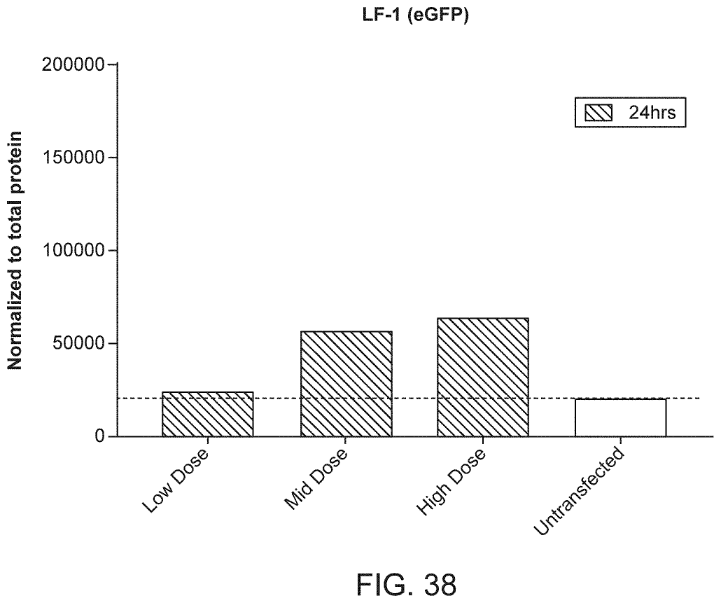

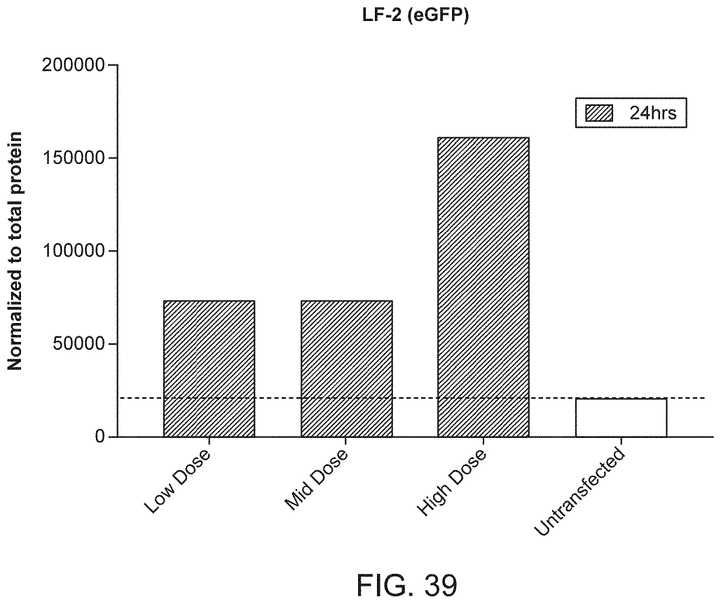

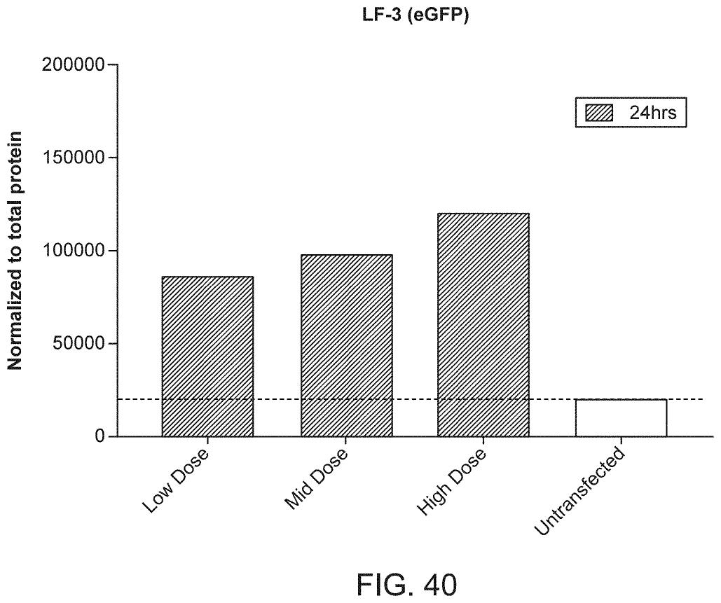

In tandem with the experiment of Example 28, the mRNA-lipid formulations were further tested for their ability to effectively express protein in human lungs in a CF subject. Per the protocols described in Example 2, an extracted set of human lungs from a CF subject was received and insufflated with low-melting temperature agarose. A conical piece was excised out and 250-micron slices were generated using a slice microtome. The slices were incubated, and cell culture media was changed several times to remove the excess of agarose. The slices were then incubated with different eGFP mRNA-lipid formulations at three different dose levels (low, mid and high). The lipid formulations used were LF-1, LF-2, LF-3 as described in Example 28. In addition, a sample of untransfected lung extract was tested as a negative control. Cell viability was monitored through the entire incubation process and was maintained for all the formulations and doses analyzed. 24 hours post incubation, samples were processed for WB and analyzed for eGFP expression. The eGFP band was quantified (normalized to total protein) and plotted as shown in FIGS. 38 (LF-1), 39 (LF-2), and 40 (LF-3). All the formulations analyzed showed a dose-dependent increase in eGFP expression levels, indicating that the lipid formulations effectively transduced a human lung matrix of a CF subject, resulting in protein expression from transduced mRNA.

### Example 30: Selected hCFTR mRNAs Showed Higher Expression than a Comparative Sequence

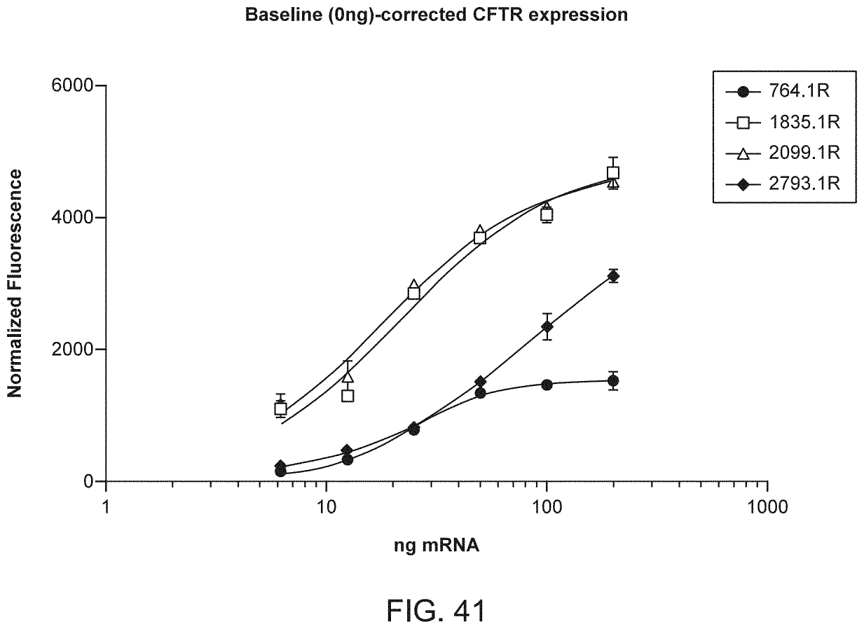

The hCFTR mRNAs of the present disclosure were tested in comparison to a hCFTR mRNA sequence described in the art. CFBE cells were transfected with unformulated codon-optimized hCFTR mRNAs (construct 1835.1 having a sequence of SEQ ID NO: 53, construct 2099.1 having a sequence of SEQ ID NO: 72), the reference wild-type sequence (construct 764.1 having a sequence of SEQ ID NO: 47) and the hCFTR sequence described in U.S. Pat. Nos. 9,181,321 and 9,713,626 (listed at as SEQ ID NO: 3 therein) referred to herein as construct 2793.1 and reproduced herein for convenience as SEQ ID NO: 146. An ascending dose transfection experiment was designed and performed to determine expression levels for each of the mRNAs. The hCFTR protein expression was measured by WB per the protocols described in Example 1 using an hCFTR-specific primary antibody and the results are shown in FIG. 41. As can be seen, the data shows that compound 2793.1 and the wild-type reference sequence had similar expression levels, at least at low doses. However, both construct 1835.1 (SEQ ID NO: 53) and construct 2099.1 (SEQ ID NO: 72) expressed significantly higher levels of protein at any given dose. Thus, the hCFTR constructs of the present disclosure demonstrate superior translation efficiency.

### Example 31: Delivery of mRNA-Lipid Formulations to Ferret Epithelial Cells

The physiology and tracheobronchial tree of ferret airways are more similar to human airways than those of mice. Unlike rodents, ferrets develop Cystic Fibrosis (CF) lung disease that is similar to CF lung disease observed in humans. Therefore, it was important to generate proof of concept of delivery in an airway model with greater similarity to human airways, such as the ferret. The ROSA26TG ferret model constitutively expresses TdTomato in the airways. Upon CRE recombination, TdTomato expression is turned off and Enhanced Green Fluorescent Protein (eGFP) expression is activated. A 0.6 mg/ml dose of CRE mRNA-lipid formulation was delivered to ROSA26TG ferret airways using a microsprayer. Seven days after dosing, when recombination was complete, the animals were sacrificed, and the lungs were removed and analyzed by immunohistochemistry for both TdTomato and eGFP expression. DAPI was used as a counterstain.

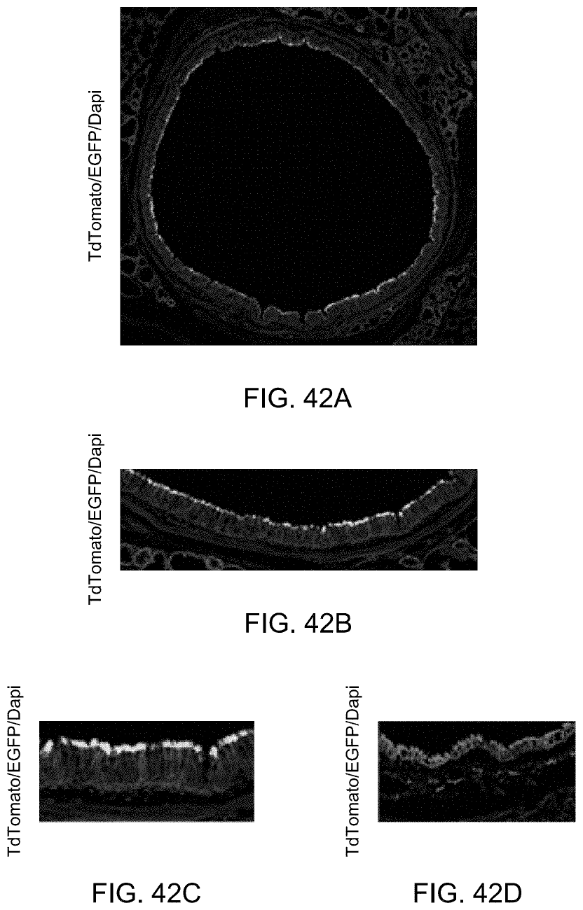

Animals treated with CRE mRNA-lipid formulation showed clear delivery of CRE mRNA to epithelial cells, as indicated by eGFP expression (FIGS. 42A-C, bright staining surrounding the airway). Thus, treatment of animals with lipid formulated-CRE mRNA resulted in clear transduction of cells in the epithelium. By contrast, untreated controls showed only TdTomato expression due to a lack of CRE recombination (FIG. 42D).

These results show efficient delivery of mRNA-lipid formulation to ferret lung epithelial cells.

### Example 32: Delivery of mRNA-Lipid Formulations to Non-Human Primate Epithelial Cells

Non-human primate (NIP) airways (e.g., the tracheobronchial tree) are more similar to human airways than those of any other species. Like humans, NHPs are nasal and mouth breathers, and pharmacologically, findings observed in an NHP by delivering an aerosolized drug are likely to be more relevant to human pathology than findings from any other species. Therefore, the NHP model was used to aerosolize lipid formulated-mRNA compounds using a face mask nebulization system.

A 1 mg/ml dose of aerosolized lipid formulated-TdTomato mRNA was delivered to non-human primate (NHP) airways using a face mask exposure system. The NHPs were exposed to the mRNA formulation for 120 minutes. Forty-eight hours post-administration, the animals were sacrificed, and the lungs were removed and analyzed by immunohistochemistry for expression of the TdTomato protein. Cresyl Violet was used as a counterstain.

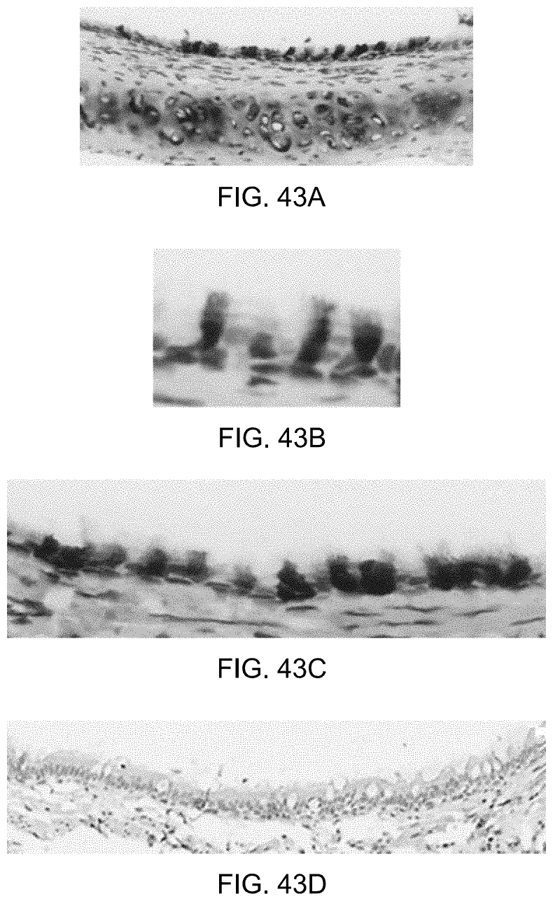

NHPs treated with lipid formulated-TdTomato mRNA showed clear mRNA delivery to ciliated-like cells in epithelial airways, as seen by dark staining of cells lining the airway (FIGS. 43A-C). By contrast, NHPs treated with PBS control showed no TdTomato expression (FIG. 43D).

These results show efficient delivery of mRNA-lipid formulation to NHP lung epithelial cells.

### Example 33: Delivery of mRNA-Lipid Formulations to Ciliated Epithelial Cells of Ferret Airways

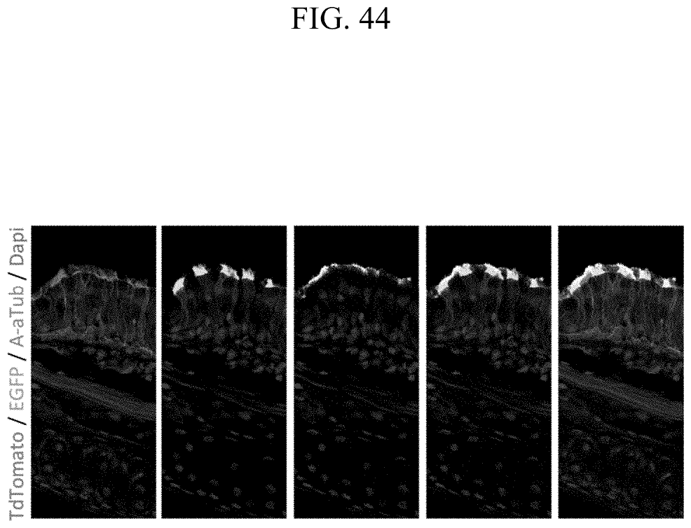

Two 0.6 mg/ml doses, separated 48 hours apart, of lipid formulated-CRE mRNA were delivered to ROSA26TG ferret airways using a microsprayer to provide for robust delivery for immunofluorescence analysis. Upon CRE recombination, TdTomato expression is turned off and eGFP expression is activated. Seven days after delivery, when recombination is complete, animals were sacrificed, and lungs were removed and analyzed by immunofluorescence for co-localization of TdTomato, eGFP, and the ciliated cell marker Acetylated Alpha-Tubulin (A-aTub; FIG. 44). DAPI was used as a counterstain (FIG. 44, all panels). FIG. 44: first panel from left—TdTomato; second panel from left—eGFP; third panel from left—A-aTub; fourth panel from left—overlay of eGFP, A-aTub, and DAPI; fifth panel from left—overlay of TdTomato, eGFP, A-aTub, and DAPI.

TdTomato staining was seen throughout the tissue section, including in cells lining the airways (FIG. 44, first and fifth panels from left). eGFP and A-aTub staining was seen in cells lining the airways (FIG. 44, bright staining, second and third panels from left, respectively). Co-localization of eGFP and Acetylated-Alpha Tubulin indicated efficient delivery to ciliated epithelial cells (FIG. 44, fourth and fifth panels from left).

These results show efficient delivery of mRNA-lipid formulation to ciliated cells of ferret lung epithelium.

### Example 34: Intranasal Administration of Lipid-Formulated hCFTR

This example illustrates intranasal administration of lipid-formulated hCFTR mRNA in a Class I CFTR knock-out (KO) mouse model.

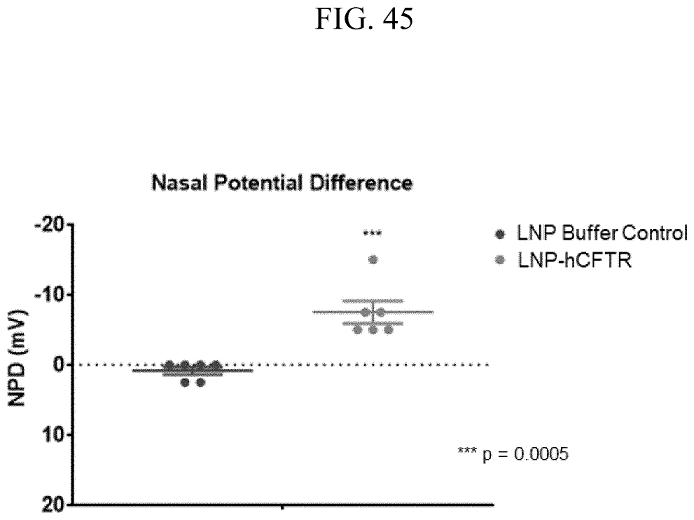

An codon-optimized hCFTR mRNA formulated as a lipid nanoparticle (LNP) was administered intranasally to CFTR KO mice at a dose of 1 mg/kg/day on two days. LNP buffer was used as a negative control. 72 hours after administration, nasal potential difference (NPD) was measured to determine the voltage across the nasal epithelium. All animals that had received hCFTR mRNA showed a statistically significant increase in chloride channel activity as compared to controls that did not show activity (FIG. 45).

These results show that intranasal delivery of lipid-formulated hCFTR mRNA results in expression of functional hCFTR in the nasal epithelium as seen by chloride channel activity.

### Example 35: Comparison of Single and Multiple Administrations of LNP-hCFTR mRNA

This example illustrates the effect of single versus multiple administrations of LNP-hCFTR mRNA.

A Class I CFTR knockout (KO) mouse model (Hodges et al., Genesis 46, 546-552, 2008) was used to compare the effect of administration of a single higher or full dose of LNP-hCFTR mRNA versus administration of multiple lower doses that resulted in administration of the same total amount of LNP-hCFTR as compared to the higher or full dose. LNP-hCFTR mRNA was administered intranasally at a single dose of 2 mg/kg or at multiple doses of 0.4 mg/kg on each of five consecutive days. 72 hours post-administration, nasal potential difference (NPD) was measured.

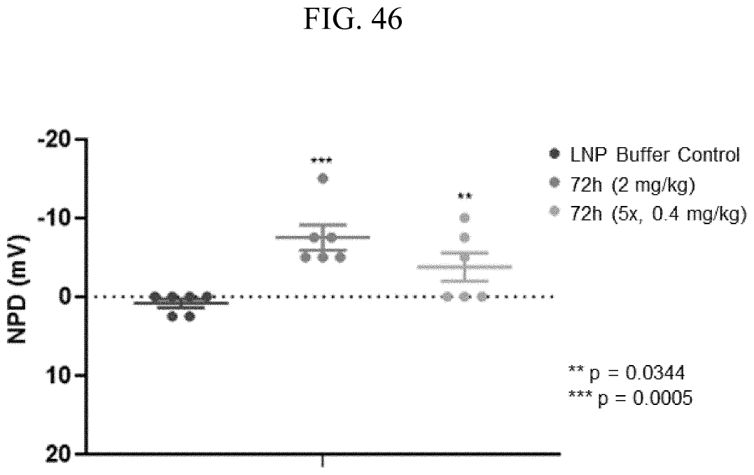

Animals that had received a single full dose or multiple lower doses of LNP-hCFTR mRNA showed significant levels of chloride channel activity as compared to controls that had received LNP buffer (FIG. 46). All animals that had received a single full dose of LNP-hCFTR mRNA showed chloride channel activity, whereas 50% of animals that had received multiple lower doses of LNP-hCFTR mRNA showed chloride channel activity (FIG. 46). Thus, without being limited by theory, administration of the single full dose of LNP-hCFTR mRNA resulted in greater efficacy of delivery and chloride channel activity as compared to administration of multiple lower doses of LNP-hCFTR mRNA. As a control, animals that had received LNP buffer did not show chloride channel activity.

These data show that administration of a single full dose of LNP-hCFTR mRNA results in greater efficacy of functional hCFTR expression in nasal epithelium as compared to administration of multiple smaller doses in a CFTR KO model.

### Example 36: Expression of Functional hCFTR in a CFTR-Deficient Ferret Cells

This example illustrates LNP-mediated delivery of hCFTR mRNA to CFTR-deficient ferret cells.

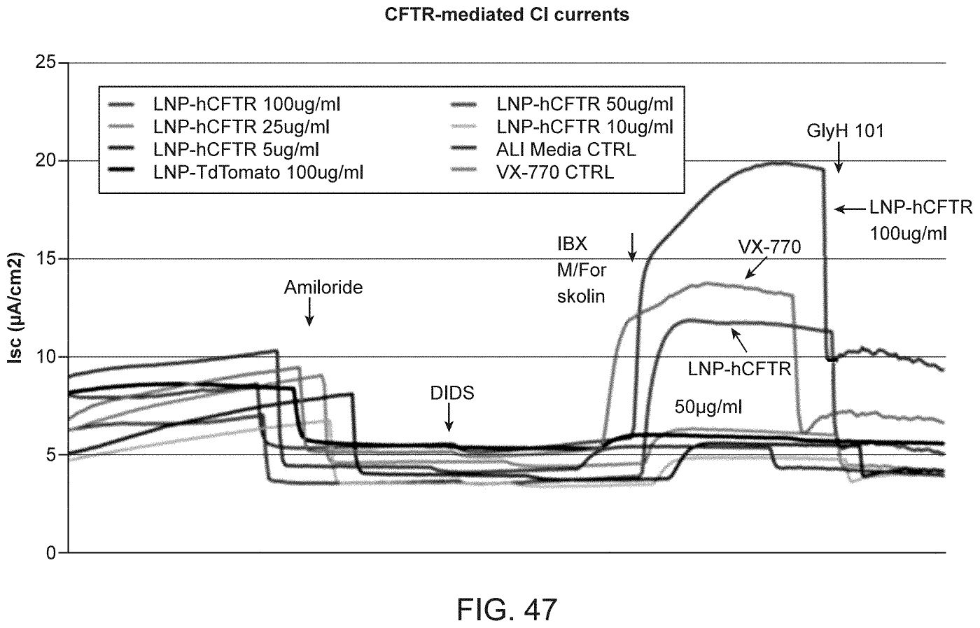

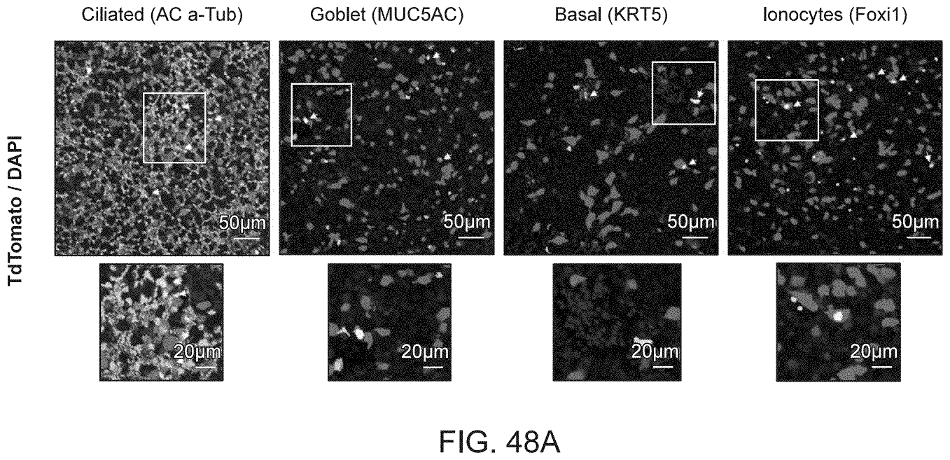

LNP-hCFTR mRNA was used to transduce ferret bronchial epithelial (FBE) cells carrying a G551D CFTR mutation. FBE cells were cultured at the air-liquid interface (ALI). LNP-mRNA formulations were administered apically at doses ranging from 5 μg/ml to 100 μg/ml. VX770 was used at a dose of 3 μM for the purpose of comparison. Untreated cells and LNP-TdTomato mRNA-treated cells were used as controls. 48 hours post-administration, transepithelial chloride currents (TECC) were measured (FIG. 47). Amiloride was used to inhibit the epithelial sodium channel (ENaC). Forskolin was used to activate CFTR-dependent channels, followed by use of GlyH 101 to inhibit the channels.

TECC data showed a dose response for increasing amounts of LNP-hCFTR mRNA administered, with the highest doses tested resulting in comparable or higher CFTR activity than that seen with a 3 μM dose of VX770.

These results show that LNP-mediated delivery of hCFTR mRNA results in expression of functional CFTR proteins in CFTR-deficient ferret epithelial cells, a relevant model with lung physiology and airway cell biology more similar to humans than to mouse.

### Example 37: LNP-mRNA Delivery to Human Bronchial Epithelial Cells (HBE)

This example illustrates LNP-mediated mRNA delivery to human bronchial epithelial (HBE) cells.

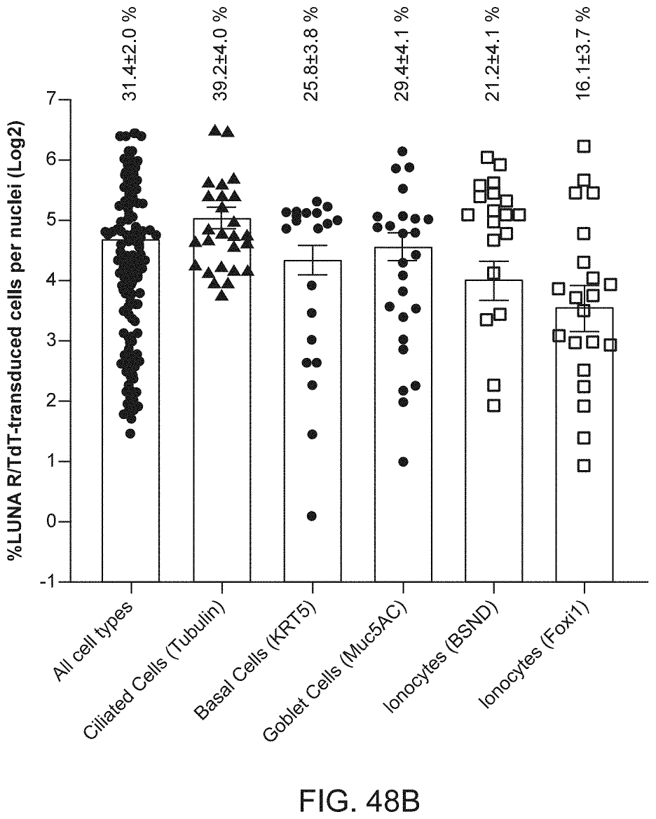

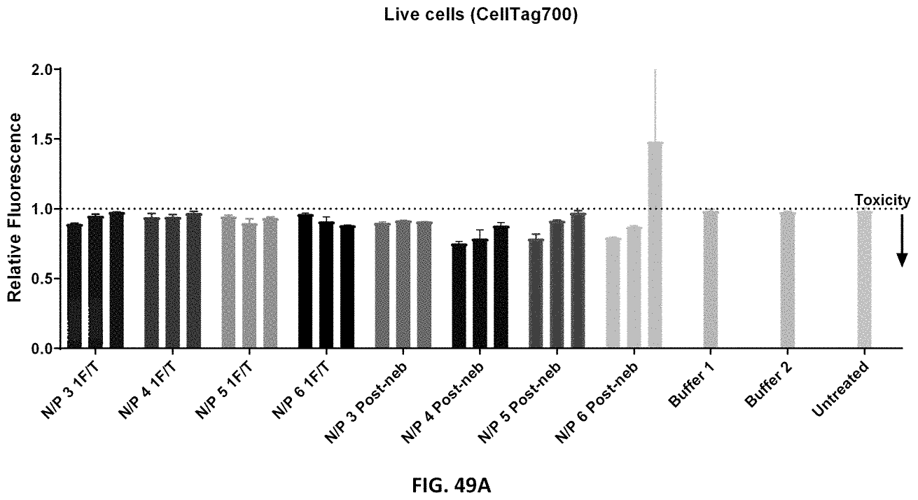

LNP-TdTomato mRNA was used to transduce human bronchial epithelial (HBE) cells derived from three non-CF human donors. HBE cells were cultured at the air-liquid interface (ALI). A single LNP-mRNA dose was administered apically in each well, with each administration performed in triplicate. 24 hours post-administration, cells were processed for immunocytology using antibodies for TdTomato and the indicated specific epithelial cell markers (FIG. 48A). Specifically, anti-acetylated alpha-tubulin (Ac a-Tub) antibody was used to stain ciliated cells, anti-MUC5AC antibody was used to stain goblet cells, anti-cytokeratin 5/KRT5 antibody was used to stain basal cells, and anti-Foxi1 antibody was used to stain ionocytes.

Each cell marker tested showed co-localization with TdTomato (FIG. 48A), consistent with the ability of LNPs to deliver mRNA to multiple epithelial cell types. The percentage of transduced TdTomato-positive cells within each epithelial cell population tested in culture is shown in FIG. 48B, further illustrating efficient delivery to different human epithelial cells.

These results show efficient LNP-mediated mRNA delivery to multiple human epithelial cell types.

### Example 38: Preparation of mRNA Lipid Nanoparticle Formulations

This example illustrates general methods for preparing formulations containing mRNA-encapsulated lipid nanoparticles evaluated in Examples 39 to 45.

Lipid excipients (DOTAP, ATX12, DSPC, cholesterol and PEG2000-DMG) were dissolved in 200 proof ethanol until complete dissolution. After filtration with a 0.2 μm polyethersulfone (PES) membrane filter, the lipid solution was rapidly mixed with aqueous hCFTR mRNA solution prepared in citrate buffer, pH 3.5 or 4.0, at a flow rate ratio of 1:3 (v/v) using a T-shaped mixing module. The rate of addition for each solution was controlled using a high-pressure piston pump (Knauer), with the lipid and mRNA solutions being added at a flow rate of 75 and 225 mL/min, respectively. The two streams converged in a stainless-steel mixing module at a total flow rate 300 mL/min. The resulting formed lipid nanoparticles were stabilized by diluting with 45 mM phosphate buffer, pH 6.0, at a dilution ratio of 1:1.5 to 1:2.5, followed by further dilution with HEPES (4-(2-hydroxyethyl)-1-piperazineethanesulfonic acid) or TRIS (tris(hydroxymethyl)aminomethane) buffer at a dilution ratio of 1:2 to 1:3. To concentrate the formulation, the diluted formulations were processed with tangential flow filtration (TFF) using a PES hollow fiber membrane (100 KDa MWCO) to ensure ethanol removal and buffer exchange with HEPES or TRIS with the following buffer. The HEPES buffer used in dilution and diafiltration contained 0-200 mM HEPES, 0 mM to 200 mM NaCl and 0% to 9% (w/v) sucrose at pH 7.8 to 8.2. The TRIS buffer used in dilution and diafiltration contained 20 mM to 50 mM TRIS, 50 mM NaCl and 9% (w/v) sucrose at pH 8.0. After confirming ethanol removal via Alco-Screen Alcohol Test Strip analysis, the concentrated solution was filtered with the 0.2 μm PES filter to remove potential larger particulates and microbiological contaminants, which was also referred to as “bulk formulation.” An in-process RNA concentration analysis was performed by a Ribogreen assay (described below), and the formulation concentration was adjusted to the final target concentration with a storage buffer containing HEPES (0-200 mM) or TRIS (20-50 mM), pH 7.8 to 8.2), 0 mM to 200 mM NaCl plus 0-9% Sucrose (w/v) and 0% to 5% glycerol as cryoprotectant. After sterile filtration, the formulation was aseptically filled into glass vials and stored frozen at −70° C.

### Dynamic Light Scattering

The average particle size (PS) and polydispersity index (PDI) of lipid nanoparticle formulations was measured by dynamic light scattering on a Malvern Zetasizer Nano ZS (United Kingdom). Z average (or Z mean) is the intensity weighted mean hydrodynamic size of the ensemble collection of particles measured by dynamic light scattering (DLS).

### RiboGreen Assay

The encapsulation efficiency of the lipid nanoparticle formulations was characterized using the RiboGreen fluorometric assay. RiboGreen is a proprietary fluorescent dye (Molecular Probes/Invitrogen a division of Life Technologies, now part of Thermo Fisher Scientific of Eugene, Oreg., United States) that is used in the detection and quantification of nucleic acids, including both RNA and DNA. In its free form, RiboGreen exhibits little fluorescence and possesses a negligible absorbance signature. When bound to nucleic acids, the dye fluoresces with an intensity that is several orders of magnitude greater than the unbound form. The fluorescence can then be detected by a sensor (fluorimeter) and the nucleic acid can be quantified.

mRNA Purity by Fragment Analyzer

The purity of the mRNA encapsulated in the lipid nanoparticle formulations was characterized using a fragment analyzer instrument and Fragment Analyzer Kit (Advanced Analytical Technologies, Inc.). The fragment analyzer instrument utilizes a parallel capillary gel electrophoresis system that can separate RNA fragments based on their sizes, with shorter fragments migrating faster than longer fragments. RNA quantification was facilitated by an intercalating dye included in the Fragment Analyzer Kit. The retention time of the full-length RNA fragment was determined with the help of an RNA ladder and a reference standard. The purity of the sample was calculated by the software based on customized global criteria.

### Example 39: Formulation Optimization by Adjusting N/P Ratio

This example illustrates the optimization of the ratio of ionizable amine groups (N) in the formulation lipids to phosphate groups (P) in the negatively charged CFTR mRNA targeted for encapsulation (the “N/P ratio”).

Physical Characteristics of Lipid Formulations with Target N/P Ratios of 3 to 5

Samples containing lipid encapsulated mRNA formulations were prepared essentially as described in Example 38, with the following exceptions. The lipids were rapidly mixed with aqueous hCFTR mRNA solution prepared in citrate buffer, pH 3.5, at a flow rate ratio of 1:3 (v/v) using T-shaped mixing module. The formed lipid nanoparticles were stabilized by diluting with 45 mM phosphate buffer, pH 6.0, followed by buffer containing 50 mM HEPES, 50 mM NaCl and 9% (w/v) sucrose at pH 8.0. The lipid formulations contained DOTAP:ATX-012:DSPC:cholesterol:PEG2000-DMG in a mole ratio of 25:25:10:38.5:1.5. The target concentration of the hCTFR mRNA (capped) was 1.2 mg/mL, and the storage buffer added to the formed lipid nanoparticles prior to −70° C. storage was 50 mM HEPES pH 8.0, 50 mM NaCl, 9% (w/v) sucrose and 5% (w/v) glycerol.

The samples were characterized for mRNA content and percent encapsulation by a RiboGreen assay, lipid content by high performance liquid chromatography (HPLC), and particle size (PS) and polydispersity index (PDI) by dynamic light scattering on a Malvern Zetasizer Nano ZS as described in Example 38. Parameters were assessed after dilution, after concentration by tangential flow filtration (TFF) using modified polyethersulfone (mPES) hollow fiber membranes (100 kD MWCO), after filtration with 0.2 μm membrane and after 1 freeze-thaw cycle (1 F/T). Actual N/P was determined based on the actual mRNA, DOTAP and ATX-012 concentrations.

The results are shown in Table 3. Formulations having a target N/P ratio of 3 showed a particle size diameter increase of about 15 nm during the TFF process. In contrast, when the target N/P ratio was increased to 4 or 5, the particle size increased by only about 3 or 2 nm, respectively. The encapsulation efficiency of the N/P 3 formulation was also slightly lower than that observed with the N/P 4 and N/P 5 formulations.

TABLE 3
Formulations with different N/P ratios prepared with capped hCFTR mRNA.

| | | | | | | After filtration with 0.2 μm | | | | | | | | |
|---|---|---|---|---|---|---|---|---|---|---|---|---|---|---|
| | | | | | | membrane | | | | | After 1 F/T | | | |
| | After dilution | | | After TFF | | | | | | mRNA | | | | mRNA |
| | Diameter | | Encap | Diameter | | Diameter | | Encap | Actual | purity | Diameter | | Encap | purity |
| N/P | (nm) | PDI | (%) | (nm) | PDI | (nm) | PDI | (%) | N/P | (%) | (nm) | PDI | (%) | (%) |
| 3 | 87.70 | 0.10 | 95.90 | 102.30 | 0.15 | 98.99 | 0.15 | 96.70 | 4.16 | 80 | 99.67 | 0.13 | 97.6 | 79 |
| 4 | 85.33 | 0.15 | 98.3 | 88.61 | 0.16 | 87.05 | 0.08 | 98.60 | 5.33 | 81 | 87.68 | 0.12 | 99.3 | 75 |
| 5 | 85.20 | 0.13 | 99.0 | 86.77 | 0.16 | 82.94 | 0.10 | 99.00 | 7.11 | 81 | 85.90 | 0.14 | 99.7 | 80 |

In Vitro Efficacy of Pre- and Post-Nebulized Lipid Formulations with Tar2et N/P Ratios of 3 to 6 Containing tdTomato mRNA

To compare the transfection efficiency of formulations prepared with different N/P ratios in vitro, lipid nanoparticle formulations were prepared with a reporter mRNA that can express red fluorescent protein (tdTomato).

Samples containing lipid encapsulated tdTomato were prepared essentially as described above, except that the hCFTR mRNA was replaced with the tdTomato mRNA (capped), and the target N/P ratios ranged from 3 to 6. The formulations contained DOTAP:ATX-012:DSPC:cholesterol:PEG2000-DMG in a mole ratio of 25:25:10:38.5:1.5, with a target tdTomato mRNA concentration of 1.2 mg/mL (see Table 4). Further dilutions were performed as described below.

TABLE 4
Formulations with different N/P ratios prepared with capped tdTomato mRNA

| | After 1 F/T | | | | | | | | | | | |
|---|---|---|---|---|---|---|---|---|---|---|---|---|
| | Pre-N ebulization | | | | Post-Nebulization | | | | Post-Nebulization | | | |
| | (1.2 mg/mL) | | | | (0.25 mg/mL) | | | | (0.6 mg/mL) | | | |
| | Particle | | | mRNA | Particle | | | mRNA | Particle | | | mRNA |
| | Size | | Encap | purity | Size | | Encap | purity | Size | | Encap | purity |
| N/P | (nm) | PDI | (%) | (%) | (nm) | PDI | (%) | (%) | (nm) | PDI | (%) | (%) |
| 3 | 92.54 | 0.06 | 96.2 | 79 | 860 | 0.963 | 94.80 | 81 | 423.6 | 0.562 | 95.3 | 77 |
| 4 | 75.47 | 0.122 | 99.7 | 88 | 423.6 | 0.806 | 97.4 | 85 | 374.8 | 0.656 | 97.8 | 85 |
| 5 | 72.45 | 0.055 | 99.8 | 86 | 714 | 0.658 | 97.5 | 76 | 336.5 | 0.565 | 98.3 | 77 |
| 6 | 73.86 | 0.09 | 99.92 | NA | 415.9 | 0.658 | 97.4 | N/A | 320.7 | 0.529 | 98.6 | 79 |

The foregoing formulations were first diluted to 0.5 mg/mL with pH 8.0 buffer composed of 50 mM HEPES, 50 mM NaCl, 9% (w/v) sucrose and then further diluted with WFI at 1:1 volume ratio to a final concentration of 0.25 mg/mL for aerosolization with a vibrating mesh nebulizer. The resulting aerosols were then condensed in ice-cold tubes to produce liquids, which are referred to herein as post-nebulized formulations. As shown in Table 4, the mRNA encapsulation efficiency was maintained before and after nebulization in all formulations.

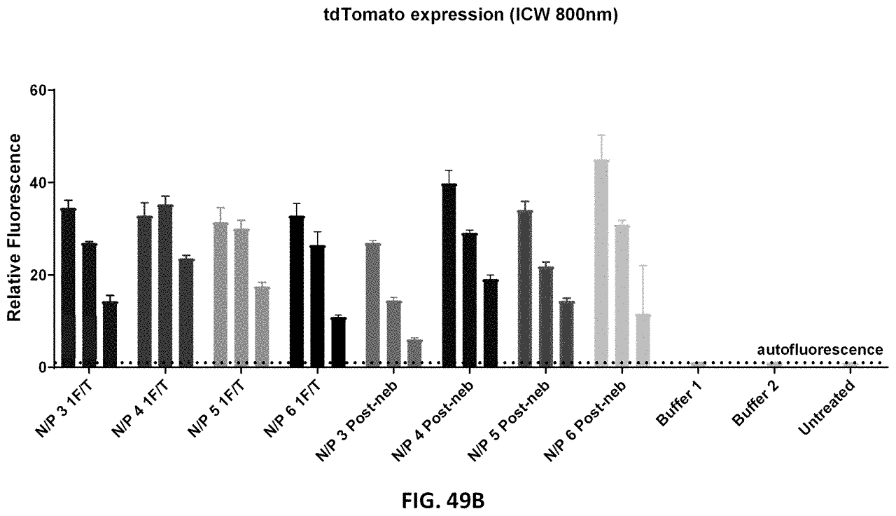

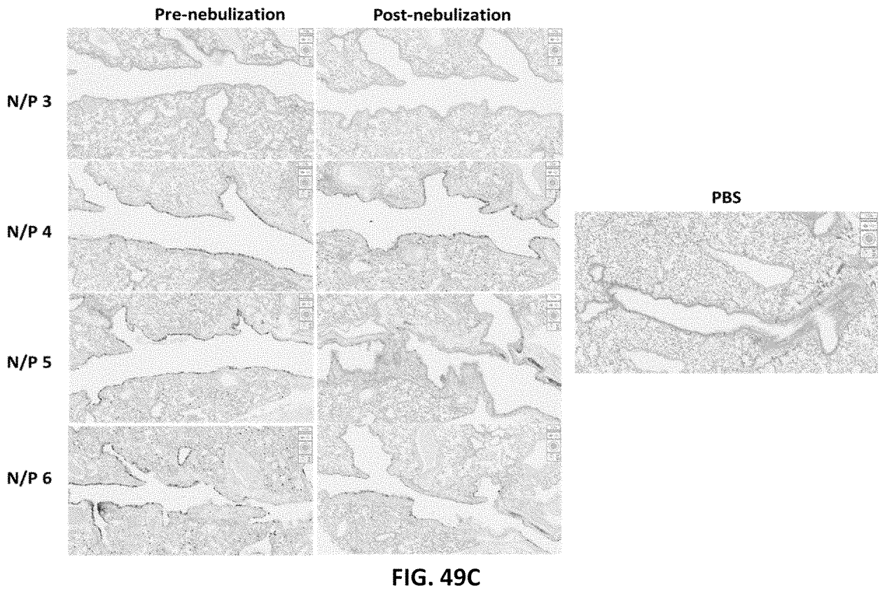

Transfection efficiency experiments were conducted in CFBE cells at three different doses (200, 100 and 50 ng) for both pre- and post-nebulized samples, as described in Example 14. The cell viability data are shown in FIG. 49A, and the transfection efficiency data are shown in FIG. 49B. Post-nebulized N/P 3 samples showed decreased transfection efficiency compared to pre-nebulized formulations. However, N/P 4 to 6 samples showed similar or slightly higher transfection efficiency compared to the corresponding pre-nebulized samples. When the stability and transfection efficiency are similar, lower N/P ratios may be more well tolerated in view of the permanently charged character of DOTAP.

In Vivo Efficacy of Lipid Formulations with Target N/P Ratios of 3 to 6

In vivo evaluation in wild-type murine lung epithelial airway was performed essentially as described in Example 14.

The tdTomato formulations with target N/P ratios ranging from 3 to 6 (Table 4) were nebulized at 0.6 mg/mL after 1:1 WFI dilution and dosed to 8 to 12 week old wild-type (Balb/c) mice intratracheally via a bolus dose at 1 mg/kg together with pre-nebulized formulations. The mice were euthanized 24 hours later, and the lungs and trachea were extracted for immunohistochemistry with tdTomato-specific antibody. FIG. 49C shows representative images from each formulation and PBS group. When the mice received pre- and post-nebulized samples at N/P 3, no obvious fluorescence was detected throughout the tissue. In contrast, animals treated with pre- or post-nebulized formulations at N/P 4 showed bright fluorescent signals in the airways, indicating N/P 4 formulation can effectively deliver its cargo to the target cells even after nebulization. At N/P 5 or 6, the mice that received pre-nebulized formulations showed good fluorescent signal, but less fluorescence signal was observed after nebulization.

### Example 40: ATX-012:DOTAP Lipid Formulation Optimization Using Design of Experiments Approach

This example illustrates the optimization of lipid compositions using a Design of Experiments approach, specifically using the orthogonal array shown in Table 5.

### Sample Preparation

Samples containing lipid encapsulated mRNA formulations were prepared essentially as described in Example 38, with the following exceptions. The lipid formulations contained varying amounts of DOTAP, ATX-012, DSPC, cholesterol and PEG2000-DMG, each with a target N/P ratio of 4, and a final uncapped hCFTR mRNA concentration of 0.5 mg/mL.

The final buffer compositions were pH 8.0 50 mM HEPES, 50 mM NaCl, 9% (w/v) sucrose and 5% (w/v) glycerol. The resulting samples were then stored at −80° C. for about 1 day and then thawed to provide samples having undergone one freeze-thaw cycle (1 F/T). Some samples underwent multiple freeze-thaw cycles (e.g., three freeze-thaw cycles; 3 F/T). Responses such as particle size (PS), polydispersity index (PDI), encapsulation efficiency and mRNA integrity were evaluated for both samples as described in the Example 38.

Freeze-thawed samples were diluted 1:1 with WFI and then nebulized as described in Example 26 using vibrating mesh for a target concentration of 0.25 mg/mL.

### Design of Experiments (DoE)

To evaluate the optimal range of each component in the formulation, formulations with varying lipid ratios of DOTAP, ATX12, DSPC and PEG2000-DMG were prepared using an L9 orthogonal array with four factors (A-D, molar ratio of DOTAP, ATX12, DSPC and PEG2000-DMG) and three levels for each factor (Table 5). The benefit of this design is that it enables the testing of each level for all factors three time by using only 9 experiments. The mol % of cholesterol provided the balance of the lipid content (100 mol % minus the sum of the mol % of other lipid components). Although the percent cholesterol was not held constant in this experimental design, our prior experience suggested that the impact of this variability would not have a substantial impact on the analysis of the four factors.

For each of the four factors, the mean value for each variable (e.g., each A1, A2, A3, B1, etc.) was determined for each level (K1, K2, K3), and the observed difference between the highest and lowest mean value was determined and defined as range (R). Higher R values indicate a higher importance of the factor (i.e., a greater impact on the measured response) and provide a basis for a ranking.

After 1 F/T, smaller particle size, smaller PDI, higher encapsulation efficiency, and higher mRNA purity were preferred outcomes.

TABLE 5
DoE orthogonal array for ATX-012:DOTAP
formulation optimization.

| | | | | | Factor D: | | |
|---|---|---|---|---|---|---|---|
| | Factor A: | Factor B: | Factor C: | | PEG- | | |
| Exp | DOTAP | ATX-012 | DSPC | | DMG | | Cholesterol |
| No. | (mol %) | (mol %) | (mol %) | | (mol %) | | mol % |
| 1 | A1 (20) | B1 (20) | C1 | (7) | D1 | (1.5) | 51.5 |
| 2 | A1 (20) | B2 (25) | C2 | (10) | D2 | (3) | 42 |
| 3 | A1 (20) | B3 (30) | C3 | (13) | D3 | (5) | 32 |
| 4 | A2 (25) | B1 (20) | C2 | (10) | D3 | (5) | 40 |
| 5 | A2 (25) | B2 (25) | C3 | (13) | D1 | (1.5) | 35.5 |
| 6 | A2 (25) | B3 (30) | C1 | (7) | D2 | (3) | 35 |
| 7 | A3 (30) | B1 (20) | C3 | (13) | D2 | (3) | 34 |
| 8 | A3 (30) | B2 (25) | C1 | (7) | D3 | (5) | 33 |
| 9 | A3 (30) | B3 (30) | C2 | (10) | D1 | (1.5) | 28.5 |
| K1 | A1 (20) | B1 (20) | C1 | (7) | D1 | (1.5) | |
| K2 | A2 (25) | B2 (25) | C2 | (10) | D1 | (3) | |
| K3 | A3 (30) | B3 (30) | C3 | (13) | D1 | (5) | |

### Results

The results of particle size, PDI, mRNA encapsulation efficiency and mRNA purity after 1 F/T and post-nebulization are shown in the Table 6. According to Table 6, all formulations showed encapsulation efficiency >9000, and no significant decrease was observed in corresponding post-nebulized formulations.

TABLE 6
Particle size, PDI, mRNA encapsulation efficiency and mRNA
purity of the formulations at 1 F/T and post-nebulization.

| | | | | | | | | | After 3 |
|---|---|---|---|---|---|---|---|---|---|
| | | | | | | | | | F/T |
| | | | | | | | | | Particle |
| | After 1 F/T | | | | Post-nebulization | | | | size |
| | Particle | | | mRNA | Particle | | | mRNA | increase |
| Experiment | size | | Encap | purity | Size | | Encap | purity | compared |
| No. | (nm) | PDI | (%) | (%) | (nm) | PDI | (%) | (%) | to bulk |
| 1 | 80.30 | 0.104 | 98.5 | 77 | 1013.2 | 0.97 | 94.63 | 67 | 3.45 |
| 2 | 84.44 | 0.151 | 96.8 | 74 | 340 | 0.58 | 93.86 | 68 | 10.16 |
| 3 | 80.79 | 0.127 | 94.7 | 64 | 218.45 | 0.76 | 93.26 | 54 | 6.72 |
| 4 | 70.18 | 0.149 | 98.9 | 74 | 211 | 0.82 | 96.86 | 65 | 3.49 |
| 5 | 87.66 | 0.078 | 98.8 | 78 | 508.45 | 0.76 | 96.79 | 68 | 2.55 |
| 6 | 81.17 | 0.143 | 95.8 | 72 | 419.55 | 0.61 | 95.69 | 57 | 13.27 |
| 7 | 70.16 | 0.15 | 99.5 | 76 | 308.3 | 0.55 | 98.15 | 73 | 2.68 |
| 8 | 71.68 | 0.163 | 97.9 | 72 | 248.6 | 0.59 | 97.31 | 67 | 5.46 |
| 9 | 85.93 | 0.125 | 99.2 | 76 | 690.25 | 0.75 | 97.37 | 70 | 3.75 |

Results of orthogonal array DoE data are summarized in Tables 7 to 10.

As shown in Table 7, the biggest factor affecting particle size was the PEG-DMG mol %. Smaller particle size was observed with higher PEG-DMG mol % in both 1 F/T and post-nebulized samples. However, less particle size growth was observed with the lowest PEG-DMG mol % when the formulations were subjected to 3 F/T cycles. Smaller and less particle size growth after 3 F/T was observed with increased DSPC mol %. At 1 F/T samples, increased DOTAP mol % decreased particle size, whereas increased ATX-012 mol % increased particle size.

As shown in Table 8, no PDI difference was observed at different levels of each factor in 1 F/T samples. Larger PDI was observed in all post-nebulized samples compared to 1 F/T samples.

As shown in Table 9, higher DOTAP mol % or lower ATX-012 mol % displayed higher encapsulation efficiency in both 1 F/T and post-nebulized formations.

As shown in Table 10, lower PEG-DMG mol %, lower ATX-012 mol % and higher DOTAP mol % showed superior mRNA purity in both 1 F/T and post-nebulized samples.

TABLE 7
Particle size analysis by orthogonal array design experiment.

| | | | | | PEG- |
|---|---|---|---|---|---|
| | | DOTAP | ATX-012 | DSPC | DMG |
| Particle Size (nm) | | (mol %) | (mol %) | (mol %) | (mol %) |
| 1 F/T | K1 | 81.84 | 73.55 | 77.72 | 84.63 |
| | K2 | 79.67 | 81.26 | 80.18 | 78.59 |
| | K3 | 75.92 | 82.63 | 79.54 | 74.22 |
| | Range | 5.92 | 9.08 | 2.47 | 10.41 |
| | Ranking | 3 | 2 | 4 | 1 |
| | Smaller better | A3 | B1 | C1-C3 | D3 |
| | Preference | 30% | 20% | 7-13% | 5% |
| Post-neb | K1 | 523.9 | 510.8 | 560.5 | 737.3 |
| | K2 | 379.7 | 365.7 | 413.8 | 356.0 |
| | K3 | 415.7 | 442.8 | 345.1 | 226.0 |
| | Range | 144.22 | 145.15 | 215.38 | 511.28 |
| | Ranking | 3 | 3 | 2 | 1 |
| | Smaller better | A2 | B2 | C3 | D3 |
| | Preference | 25% | 25% | 13% | 5% |
| Particle | K1 | 6.777 | 3.207 | 7.393 | 3.250 |
| Size | K2 | 6.437 | 6.057 | 5.800 | 8.703 |
| growth | K3 | 3.963 | 7.913 | 3.983 | 5.223 |
| after 3 F/T | Range | 2.81 | 4.71 | 3.41 | 5.45 |
| compared | Ranking | 4 | 2 | 3 | 1 |
| to bulk | Smaller better | A3 | B1 | C1 | D3 |
| | Preference | 30% | 20% | 13% | 1.5% |

TABLE 8
PDI analysis by orthogonal array design experiment.

| | | | | | PEG- |
|---|---|---|---|---|---|
| | | DOTAP | ATX-012 | DSPC | DMG |
| PDI | | (mol %) | (mol %) | (mol %) | (mol %) |
| 1 F/T | K1 | 0.13 | 0.13 | 0.14 | 0.10 |
| | K2 | 0.12 | 0.13 | 0.14 | 0.15 |
| | K3 | 0.15 | 0.13 | 0.12 | 0.15 |
| | Range | 0.02 | 0.00 | 0.02 | 0.05 |
| | Ranking | 2 | 3 | 2 | 1 |
| | Smaller better | A3 | B1 | C2 | D3 |
| | Preference | No preference | | | |
| Post-neb | K1 | 0.770 | 0.782 | 0.722 | 0.827 |
| | K2 | 0.730 | 0.645 | 0.716 | 0.581 |
| | K3 | 0.629 | 0.703 | 0.691 | 0.722 |
| | Range | 0.14 | 0.14 | 0.03 | 0.25 |
| | Ranking | 2 | 2 | 3 | 1 |
| | Smaller better | A3 | B2 | C3 | D2 |
| | Preference | 30% | 25% | 7-13% | 3% |

TABLE 9
Encapsulation efficiency analysis by
orthogonal array design experiment.

| | | | | | PEG- |
|---|---|---|---|---|---|
| Encapsulation | | DOTAP | ATX-012 | DSPC | DMG |
| efficiency (%) | | (mol %) | (mol %) | (mol %) | (mol %) |
| 1 F/T | K1 | 96.67 | 98.97 | 97.40 | 98.83 |
| | K2 | 97.83 | 97.83 | 98.30 | 97.37 |
| | K3 | 98.87 | 96.57 | 97.67 | 97.17 |
| | Range | 2.20 | 2.40 | 0.90 | 1.67 |
| | Ranking | 2 | 1 | 4 | 3 |
| | Higher better | A3 | B1 | C1 | D3 |
| | Preference | 30% | 20% | 7-13% | 1.5% |
| Post-neb | K1 | 93.92 | 96.55 | 95.88 | 96.26 |
| | K2 | 96.44 | 95.98 | 96.03 | 95.90 |
| | K3 | 97.61 | 95.44 | 96.06 | 95.81 |
| | Range | 3.69 | 1.11 | 0.19 | 0.46 |
| | Ranking | 1 | 2 | 4 | 3 |
| | Higher better | A3 | B1 | C1 to C3 | D1 to D3 |
| | Preference | 30% | 20% | 7-13% | 1.5-5% |

TABLE 10
mRNA purity analysis by orthogonal array design experiment.

| | | | | | PEG- |
|---|---|---|---|---|---|
| | | DOTAP | ATX-012 | DSPC | DMG |
| mRNA purity (%) | | (mol %) | (mol %) | (mol %) | (mol %) |
| 1 F/T | K1 | 71.67 | 75.67 | 73.67 | 77.00 |
| | K2 | 74.67 | 74.67 | 74.67 | 74.00 |
| | K3 | 74.67 | 70.67 | 72.67 | 70.00 |
| | Range | 3.00 | 5.00 | 2.00 | 7.00 |
| | Ranking | 3 | 2 | 4 | 1 |
| | Higher better | A2 or A3 | B1 or B2 | C1 to C3 | D1 |
| | Preference | 25-30% | 20-25% | 7-13% | 1.5% |
| Post-neb | K1 | 63.00 | 68.33 | 63.67 | 68.33 |
| | K2 | 63.33 | 67.67 | 67.67 | 66.00 |
| | K3 | 70.00 | 60.33 | 65.00 | 62.00 |
| | Range | 7.00 | 8.00 | 4.00 | 6.33 |
| | Ranking | 2 | 1 | 4 | 3 |
| | Higher better | A3 | B1 or B2 | C2 | D1 |
| | Preference | 30% | 20-25% | 10% | 1.5% |

### Example 41: Further Lipid Composition Optimization by Varying the DOTAP:ATX-012 Ratio

Based on DoE analysis from Example 40, further optimization of the lipid composition was conducted by tuning the DOTAP:ATX-012 molar ratio, and maintaining the DSPC at 13 mol %, and the PEG2000-DMG at 3 mol % or 1.5 mol %. The target N/P ratio was 4.

Samples containing lipid encapsulated mRNA formulations were prepared essentially as described in Example 38. Lipid formulations containing varying amounts of DOTAP, ATX-012, DSPC, cholesterol and PEG2000-DMG, each with a target uncapped CTFR mRNA concentration of 0.5 mg/mL, were prepared. The final buffer composition was pH 8.0 50 mM HEPES, 50 mM NaCl, 9% (w/v) sucrose and 50% (w/v) glycerol. Then, physicochemical properties such as particle size (PS), PDI, mRNA encapsulation efficiency, mRNA purity were evaluated, essentially as described in Example 38. Physicochemical properties were also evaluated after diluting 1 F/T samples 1:1 (v/v) with WFI (final concentration of 0.25 mg/mL) and nebulization via vibrating mesh, as described in Example 26.

As shown in Tables 11 and 12, the formulations showed good encapsulation efficiency and mRNA purity, including post-nebulized samples. Formulations containing 20 mol % DOTAP showed slightly lower percentages of encapsulation post-nebulization than those with higher mol % DOTAP (25 mol % or 30 mol %) at the same ATX-012 mol % ratio. When the DOTAP mol % was held constant, formulations with higher mol % ATX-012 showed larger PS and slightly decreased purity in both 1 F/T and post-nebulized samples.

At constant molar ratios of DOTAP, ATX-012 and DSPC, 3 mol % PEG-DMG formulations showed much smaller particle size in both 1 F/T and post-nebulization samples than 1.5 mol % PEG-DMG formulations. These observations were consistent with the results of orthogonal array analysis in Example 40.

TABLE 11
Characterization of formulation prepared with varying DOTAP: ATX-
012 molar ratio at 3 mol % PEG with uncapped CFTR mRNA.

| | | | | | | After 1 F/T | | | | | Post-nebulization | | |
|---|---|---|---|---|---|---|---|---|---|---|---|---|---|
| Composition mol (%) | | | | | | | | | mRNA | | | | mRNA |
| | | ATX- | | | PEG- | PS | | Encap | purity | PS | | Encap | purity |
| Experiment | DOTAP | 012 | DSPC | CHOL | DMG | (nm) | PDI | (%) | (%) | (nm) | PDI | (%) | (%) |
| 1 | 20 | 15 | 13 | 49 | 3 | 68.83 | 0.161 | 98.6 | 77 | 288.3 | 0.78 | 96.1 | 71 |
| 2 | 20 | 20 | 13 | 44 | 3 | 73.47 | 0.119 | 99.4 | 79 | 242.6 | 0.75 | 96.5 | 69 |
| 3 | 20 | 25 | 13 | 39 | 3 | 78.56 | 0.12 | 98.9 | 76 | 272.6 | 0.63 | 96.0 | 67 |
| 4 | 25 | 15 | 13 | 44 | 3 | 67.20 | 0.13 | 98.5 | 73 | 295.4 | 0.71 | 97.4 | 73 |
| 5 | 25 | 20 | 13 | 39 | 3 | 71.24 | 0.169 | 99.5 | 79 | 290.4 | 0.78 | 97.9 | 73 |
| 6 | 25 | 25 | 13 | 34 | 3 | 75.18 | 0.143 | 99.3 | 79 | 302.5 | 0.66 | 97.0 | 71 |
| 7 | 30 | 20 | 13 | 34 | 3 | 72.68 | 0.161 | 99.4 | 78 | 301.5 | 0.963 | 98.4 | 74 |
| 8 | 30 | 25 | 13 | 29 | 3 | 78.18 | 0.165 | 99.4 | 79 | 324.7 | 0.732 | 97.6 | 71 |
| 9 | 25 | 25 | 10 | 37 | 3 | 72.87 | 0.153 | 99.5 | 82 | 401 | 0.583 | 97.0 | 76 |

TABLE 12
Characterization of formulation prepared with varying DOTAP: ATX-
012 molar ratio at 1.5 mol % PEG-DMG with uncapped CFTR mRNA.

| | | | | | | | | | Post-nebulization | | | |
|---|---|---|---|---|---|---|---|---|---|---|---|---|
| | | | | | | | | mRNA | | | | mRNA |
| Composition mol (%) | | | | | After 1 F/T | | | purity | PS | | Encap | purity |
| DOTAP | ATX-012 | DSPC | CHOL | PEG-DMG | PS (nm) | PDI | Encap (%) | (%) | (nm) | PDI | (%) | (%) |
| 20 | 20 | 13 | 45.5 | 1.5 | 89.4 | 0.117 | 97.8 | 81 | 552.5 | 0.73 | 94.9 | 77 |
| 20 | 25 | 13 | 40.5 | 1.5 | 99.54 | 0.1 | 96 | 83 | 689.4 | 0.79 | 93.5 | 76 |
| 25 | 15 | 13 | 45.5 | 1.5 | 72.9 | 0.203 | 99.1 | 84 | 604.4 | 0.73 | 96.7 | 80 |
| 25 | 20 | 13 | 40.5 | 1.5 | 78.11 | 0.13 | 99.1 | 83 | 608.9 | 0.66 | 97.2 | 79 |
| 25 | 25 | 13 | 35.5 | 1.5 | 85.41 | 0.106 | 98.7 | 82 | 672.7 | 0.74 | 96.9 | 77 |

To compare the transfection efficiency of each formulation, a reporter mRNA that can express red fluorescence protein (tdTomato, tdT) was encapsulated in lipid nanoparticles of the formulations containing varying molar ratios of DOTAP:ATX-012, each with a target tdTomato concentration of 1.2 mg/mL. To prepare the test samples, the formulations were first diluted to 0.5 mg/mL with pH 8.0 50 mM HEPES, 50 mM NaCl, 9% (w/v) sucrose and 5% (w/v) glycerol (w/v), and then additionally diluted to 0.25 mg/mL with WFI. The diluted formulations were further aerosolized with vibrating mesh to provide post-nebulized samples. Then, physicochemical properties such as particle size (PS), PDI, mRNA encapsulation efficiency, mRNA purity were evaluated, essentially as described in Example 38. Transfection efficiency was conducted in CFBE cells using In-Cell Western assay at three different dose (200, 100 and 50 ng), as described in Example 1.

The results of physicochemical testing are shown in Tables 13 and 14. All of the tdTomato formulations showed good encapsulation efficiency (>95%) and mRNA purity (>84%) pre- and post-nebulization.

TABLE 13
Characterization of formulation prepared with varying DOTAP: ATX-
012 molar ratio at 3 mol % PEG with tdTomato mRNA .

| | | | | | After 1 F/T | | | | Post-nebulization | | | |
|---|---|---|---|---|---|---|---|---|---|---|---|---|
| Composition mol (%) | | | | | | | | mRNA | | | | mRNA |
| | | | | PEG- | | | Encap | purity | | | Encap | purity |
| DOTAP | ATX12 | DSPC | CHOL | DMG | PS (nm) | PDI | (%) | (%) | PS (nm) | PDI | (%) | (%) |
| 20 | 20 | 13 | 44 | 3 | 64.84 | 0.14 | 99.3 | 88 | 267.3 | 0.89 | 96.2 | 85 |
| 20 | 25 | 13 | 39 | 3 | 71.38 | 0.12 | 98.4 | 90 | 267.6 | 0.66 | 95.7 | 87 |
| 25 | 15 | 13 | 44 | 3 | 54.28 | 0.18 | 99.7 | 87 | 377.5 | 0.63 | 97.4 | 87 |
| 25 | 20 | 13 | 39 | 3 | 56.35 | 0.12 | 99.5 | 91 | 373.9 | 0.80 | 97.2 | 84 |
| 25 | 25 | 13 | 34 | 3 | 62.49 | 0.13 | 98.6 | 90 | 374.6 | 0.65 | 97.2 | 85 |

TABLE 14
Characterization of formulation prepared with varying
DOTAP: ATX-012 molar ratios at 1.5 mol % PEG with tdTomato mRNA.

| | | | | | After 1 F/T | | | | Post-nebulization | | | |
|---|---|---|---|---|---|---|---|---|---|---|---|---|
| | | | | | | | | mRNA | | | | mRNA |
| Composition mol (%) | | | | | | | Encap | purity | | | Encap | purity |
| DOTAP | ATX12 | DSPC | CHOL | PEG-DMG | PS (nm) | PDI | (%) | (%) | PS (nm) | PDI | (%) | (%) |
| 25 | 25 | 10 | 38.5 | 1.5 | 75.47 | 0.122 | 99.7 | 88 | 423.6 | 0.806 | 97.4 | 85 |
| 20 | 20 | 13 | 45.5 | 1.5 | 77.25 | 0.11 | 99.2 | 85 | 334.5 | 0.95 | 96.6 | 89 |
| 20 | 25 | 13 | 40.5 | 1.5 | 87.57 | 0.10 | 98.5 | 85 | 424.7 | 0.66 | 95.2 | 88 |
| 25 | 15 | 13 | 45.5 | 1.5 | 67.71 | 0.18 | 99.4 | 85 | 637.6 | 0.69 | 97.2 | 90 |
| 25 | 20 | 13 | 40.5 | 1.5 | 70.81 | 0.13 | 99.6 | 86 | 783.0 | 0.75 | 97.2 | 87 |
| 25 | 25 | 13 | 35.5 | 1.5 | 74.87 | 0.11 | 99.5 | 87 | 611.4 | 0.68 | 97.4 | 89 |

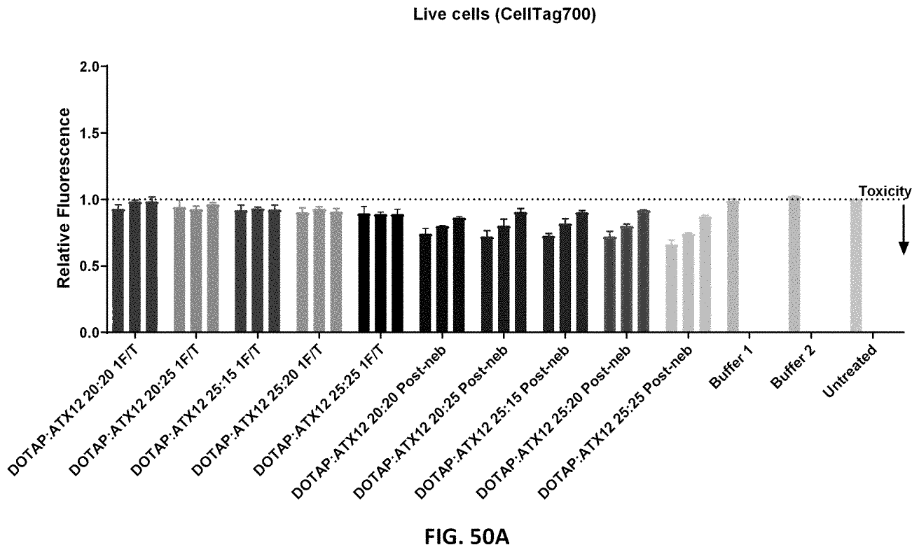

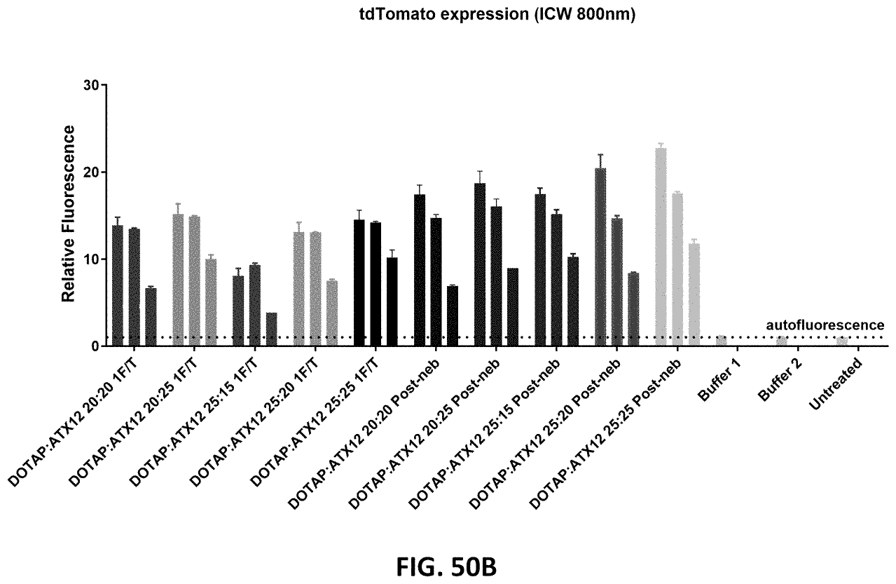

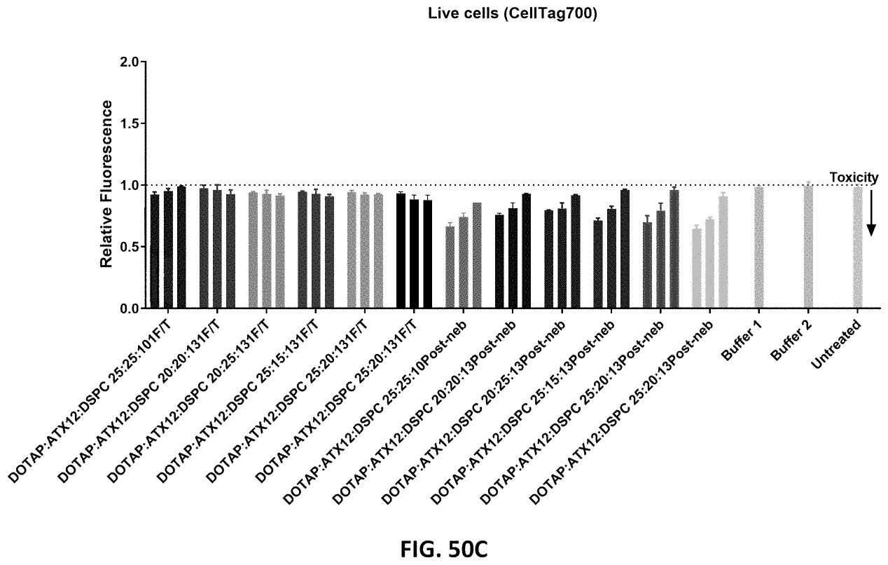

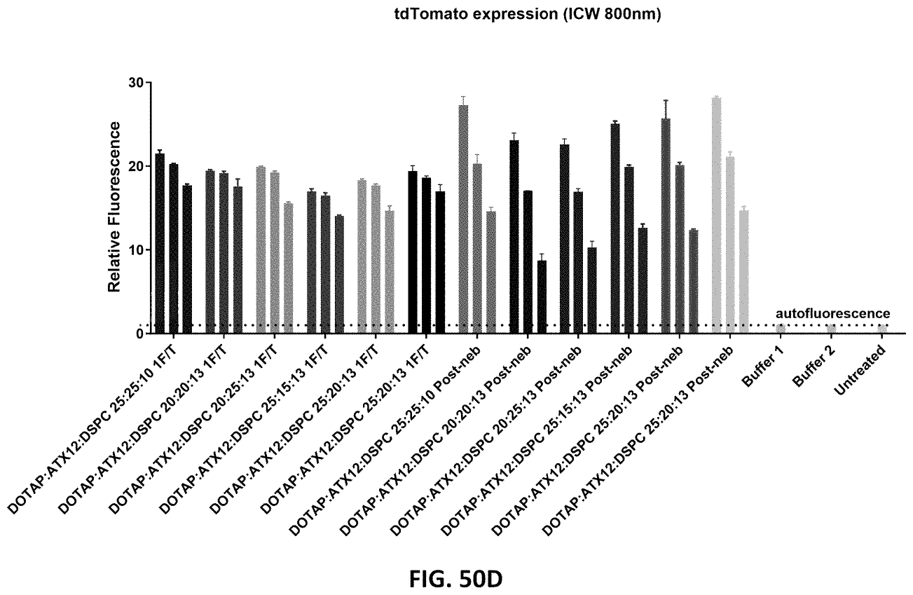

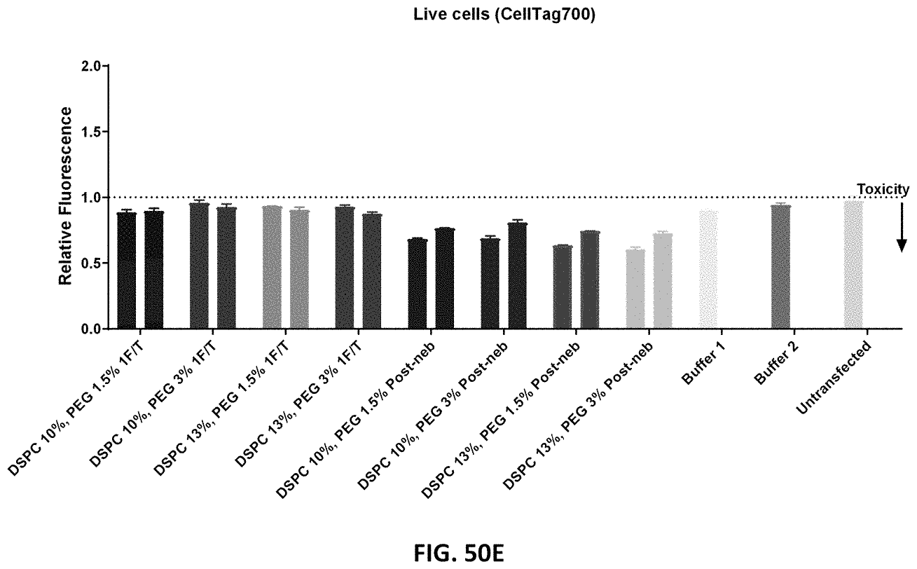

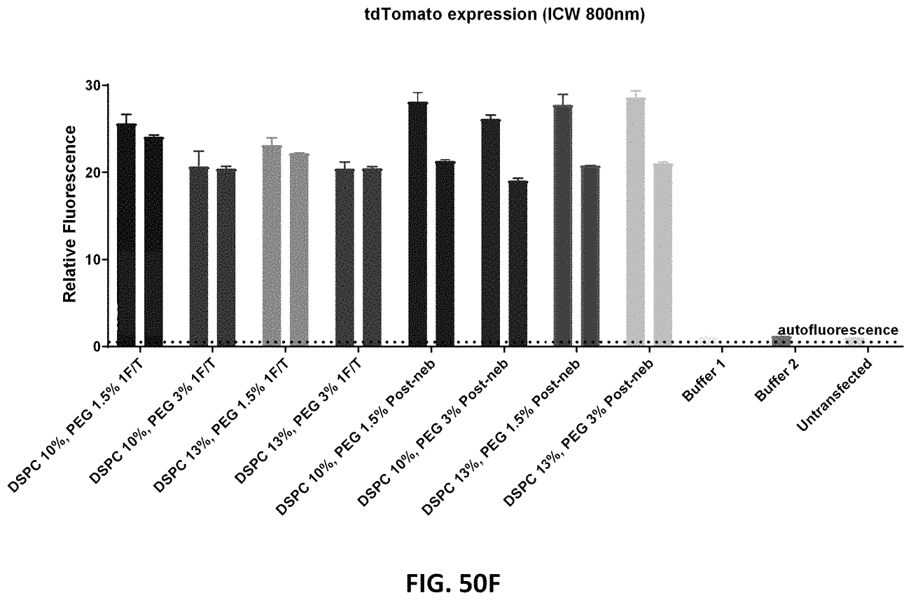

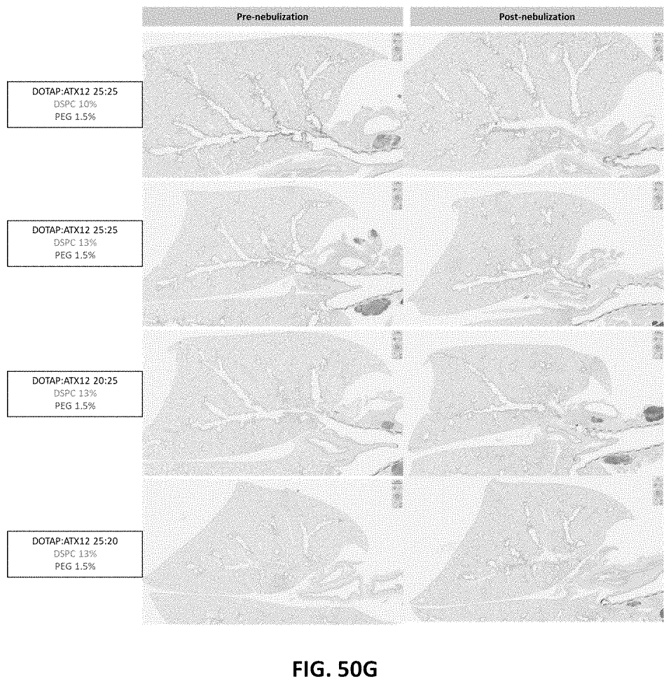

The results of transfection efficiency testing are shown in (FIGS. 50A to D). At the same dose level, no viability difference was observed between formulations with different DOTAP:ATX-012 molar ratios. For post-nebulized samples, there was a slight viability drop at the highest dose (200 ng) in all tested formulations (FIGS. 50A and C), but viability was still greater than 75% in all cases, indicating that the tested formulations tested are not cytotoxic. The transfection efficiency experiment results (FIGS. 50B and D) indicated that CFBE cells treated with pre- or post-nebulized formulations displayed good dose-dependent tdTomato expression. DOTAP:ATX-012 in a 25:25 molar ratio showed relatively higher protein expression compared to other DOTAP:ATX-012 molar ratios, regardless of PEG-DMG concentration (PEG-DMG at 3 mol % or 1.5 mol %).

Additional transfection efficiency experiments were conducted to compare tdTomato formulations prepared with DOTAP:ATX-012 25:25 molar ratio while varying DSPC mol % (10 mol % or 13 mol %) or PEG-DMG mol % (1.5 mol % or 3 mol %). As shown from the FIG. 50F, CFBE cells treated with pre-nebulized formulations containing 3 mol % PEG-DMG showed slightly lower tdTomato expression than formulations containing 1.5 mol % PEG-DMG, whereas the cells treated with post-nebulized formulations showed very similar protein expression. This confirmed that. when the DOTAP:ATX-012 molar ratio is 25:25, 13 mol % DSPC formulations exhibited good and similar transfection efficiency compared to 10 mol % DSPC formulations in vitro.

To evaluate in vivo delivery, select tdTomato formulations with varying lipid ratios were nebulized at 0.6 mg/mL after 1:1 WFI dilution and dosed to wild-type mice intratracheally via a bolus dose at 1 mg/kg, together with pre-nebulized formulations as described in Example 14. As shown in FIG. 50G, the animals treated with pre- or post-nebulized lipid nanoparticles at DOTAP:ATX-012 25:25 molar ratio showed tdTomato protein expression in lung epithelial airways. Among them, the formulations containing 10 mol % DSPC showed better fluorescent signal than formulations containing 13 mol % DSPC in either pre- or post-nebulized form. This indicates that the lipid composition DOTAP:ATX-012:DSPC:CHOL:PEG2000-DMG 25:25:10:38.5:1.5 performs best in vitro and in vivo among all tested formulations.

### Example 42: Lipid Composition Optimization of PEG-DMG Content

This example illustrates the optimization of lipid composition by adjusting the content of PEG2000-DMG.

Samples containing lipid encapsulated mRNA formulations were prepared essentially as described Example 38. The lipid solutions containing DOTAP:ATX-012:DSPC in a molar ratio of 25:25:10, plus PEG2000-DMG (1.5 mol % to 5 mol % ratio to DOTAP:ATX-012:DSPC) and cholesterol (balance, to 100 mol %) were formulated with tdTomato mRNA. The target N/P ratio was 4. The target final formulation concentration of tdTomato mRNA was 1.2 mg/mL at a final buffer composition of 50 mM HEPES, 50 mM NaCl, 9% (w/v) sucrose and 5% (w/v) glycerol. Post-nebulized formulations were prepared by nebulization of 1:1 WFI diluted 1 F/T formulations using vibrating mesh as described in Example 26 for a target concentration of 0.6 mg/mL. Additionally, formulations were first diluted to 0.5 mg/mL with pH 8.0 buffer composed of 50 mM HEPES, 50 mM NaCl, 9% (w/v) sucrose and then further diluted with WFI at 1:1 volume ratio to reach 0.25 mg/mL for aerosolization with a vibrating mesh nebulizer

The foregoing formulations were evaluated for their physicochemical properties. As shown in Tables 15 and 16, with increasing mol % PEG-DMG, smaller particle sizes were observed in both pre- and post-nebulized samples. Greater PDI was observed after dilution or TFF. Also, formulations containing 3 mol % and 5 mol % PEG-DMG showed 10 nm particle size growth during the TFF process, while formulations containing 1.5 mol % PEG-DMG showed only 5 nm particle size growth after TFF. Higher mol % PEG-DMG formulations showed slightly lower encapsulation efficiency, but overall, all the samples showed >95% encapsulation efficiency in 1 F/T or post-nebulized samples. Formulations containing 1.5 mol % PEG-DMG showed relatively higher mRNA purity after 1 F/T or post-nebulization compared to formulations containing a higher mol % PEG-DMG.

TABLE 15
Characterization of formulations prepared with 1.5 mol %, 3 mol % or
5 mol % PEG-DMG and tdTomato mRNA.

| | | | | | | | | After | | After filtration with 0.2 μm | | | |
|---|---|---|---|---|---|---|---|---|---|---|---|---|---|
| Lipid composition (mol %) | | | | | After dilution | | | TFF | | membrane | | | |
| | | | | PEG- | PS | | Encap | PS | | | | Encap | mRNA |
| DOTAP | ATX12 | DSPC | CHOL | DMG | (nm) | PDI | (%) | (nm) | PDI | PS (nm) | PDI | (%) | purity (%) |
| 25 | 25 | 10 | 38.5 | 1.5 | 70.26 | 0.127 | 98.90 | 75.75 | 0.12 | 72.02 | 0.108 | 99.7 | 89 |
| 25 | 25 | 10 | 37 | 3 | 56.42 | 0.239 | 95.50 | 66.27 | 0.22 | 62.43 | 0.106 | 97.4 | 90 |
| 25 | 25 | 10 | 35 | 5 | 49.31 | 0.223 | 93.90 | 61.12 | 0.27 | 55.63 | 0.153 | 96.1 | 90 |

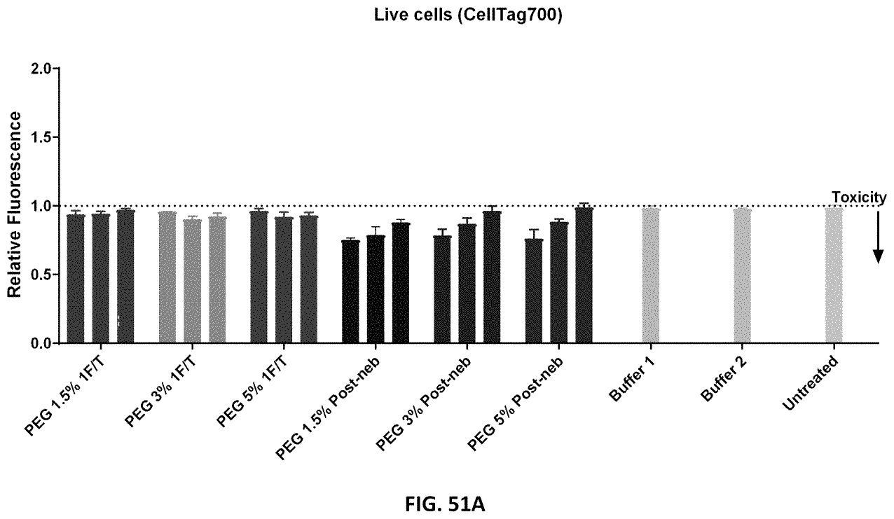

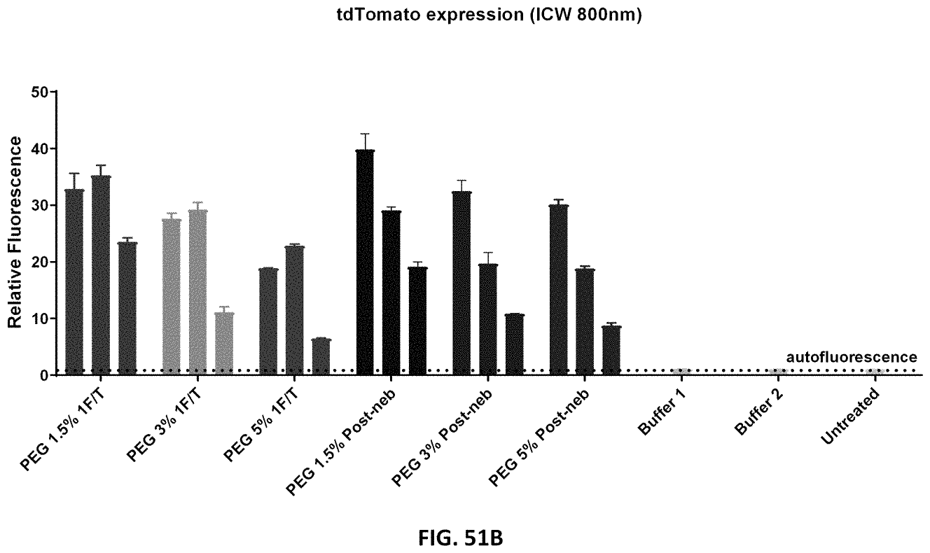

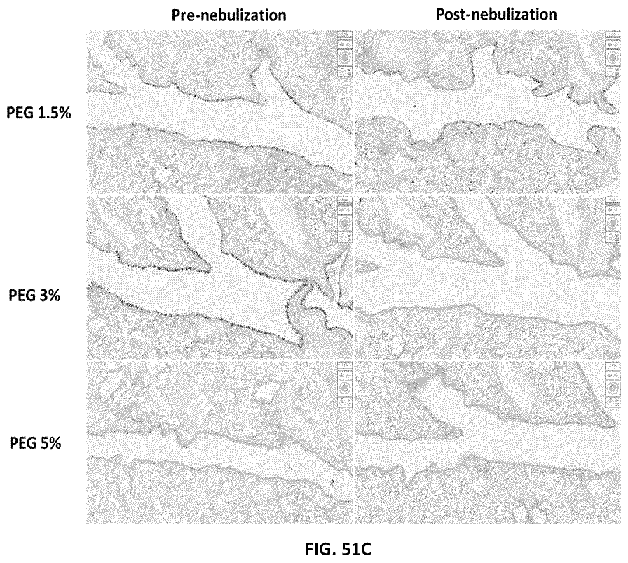

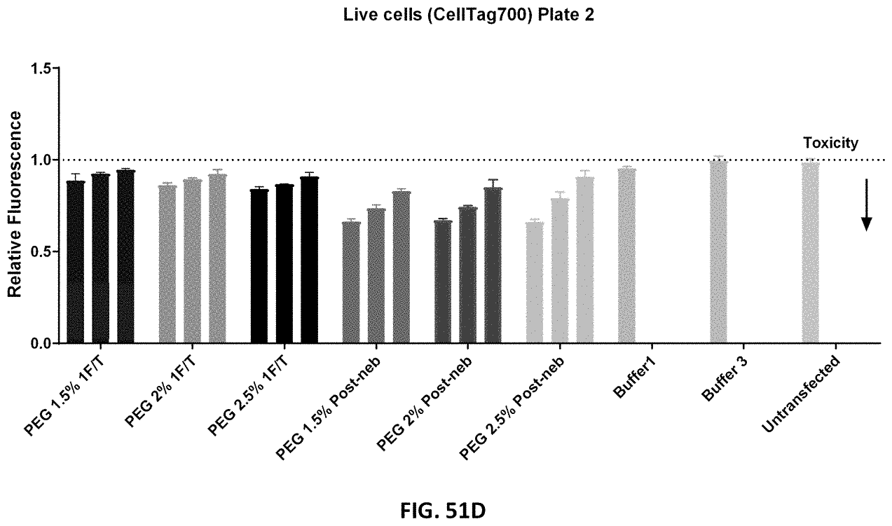

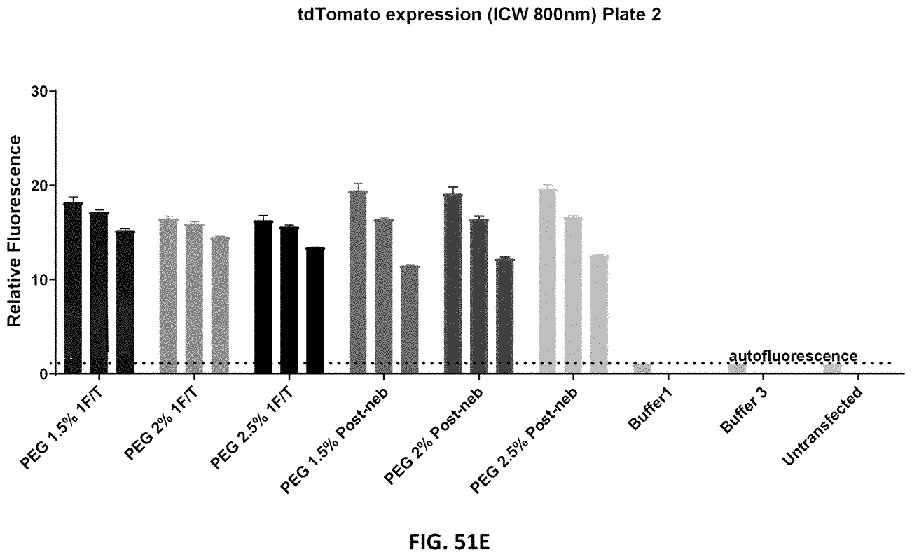

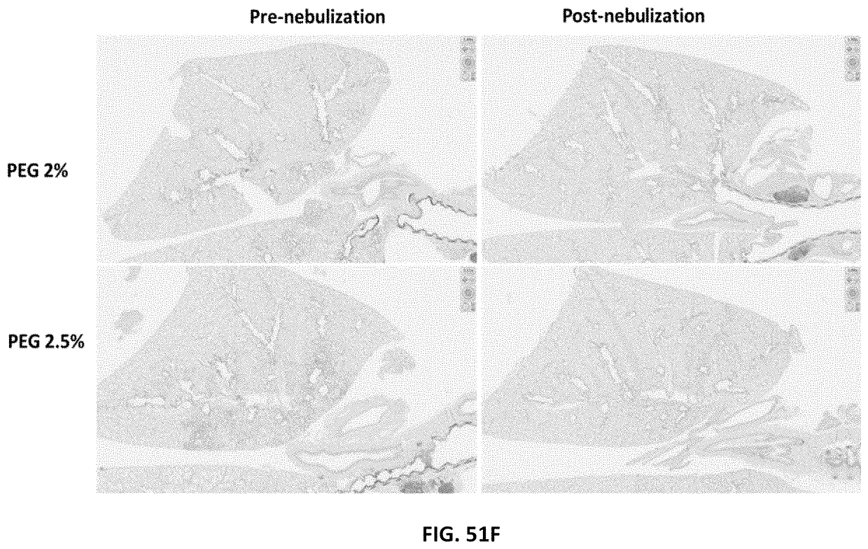

To compare the formulations for transfection efficiency, CFBE cells were treated at three different doses (200 ng, 100 ng and 50 ng) using an In-Cell Western assay, as described in Example 1. An increase in the mol % PEG-DMG corresponded with decreased protein expression in CFBE cells in both pre- and post-nebulized samples (FIG. 51B), indicating that formulations containing 1.5 mol % PEG-DMG exhibited better transfection in CFBE cells than formulations containing 3 mol % or 5 mol % PEG-DMG.

To evaluate the formulations for in vivo delivery, pre- and post-nebulized samples were dosed to wild-type mice intratracheally via a bolus dose at 1 mg/kg, as described in Example 14. As shown in FIG. 51C, the animals administrated with pre-nebulized formulations containing 1.5 mol % PEG-DMG or 3 mol % PEG-DMG showed bright fluorescent signals in lung airway epithelial cells. However, upon nebulization, the fluorescent data indicated that only the formulation containing 1.5 mol % PEG-DMG was able to achieve in vivo delivery to lung airway in the mouse model. The formulations containing 5 mol % PEG-DMG was not effective in either pre-nebulized or post-nebulized form.

TABLE 16
Characterization of pre- and post-nebulized formulations prepared
with 1.5 mol %, 3 mol % or 5 mol % PEG-DMG and tdTomato mRNA.

| | | | | | 1 F/T | | | | Post-nebulization | | | | Post-nebulization | | | |
|---|---|---|---|---|---|---|---|---|---|---|---|---|---|---|---|---|
| Lipid composition (mol %) | | | | | (Pre-nebulization) | | | | (0.25 mg/mL) | | | | (0.6 mg/mL) | | | |
| | | | | | | | | mRNA | | | | mRNA | | | | mRNA |
| | ATX- | | | PEG- | PS | | Encap | purity | PS | | Encap | purity | PS | | Encap | purity |
| DOTAP | 012 | DSPC | CHOL | DMG | (nm) | PDI | (%) | (%) | (nm) | PDI | (%) | (%) | (nm) | PDI | (%) | (%) |
| 25 | 25 | 10 | 38.5 | 1.5 | 75.47 | 0.122 | 99.7 | 88 | 509.4 | 0.708 | 97.4 | 86 | 374.8 | 0.656 | 97.8 | 85 |
| 25 | 25 | 10 | 35 | 3 | 66.27 | 0.133 | 98.6 | 80 | 491.5 | 0.723 | 97.5 | 88 | 240.7 | 0.87 | 97.7 | 77 |
| 25 | 25 | 10 | 35 | 5 | 59.97 | 0.181 | 97.5 | 80 | 385.7 | 0.703 | 96.6 | 80 | 211.3 | 0.709 | 96.8 | 72 |

To optimize the mol % PEG-DMG in the DOTAP:ATX-012:DSPC formulations, additional formulations containing 1.5 mol %, 2 mol % and 2.5 mol % PEG-DMG were prepared (see Tables 17 and 18) and compared in vitro and in vivo. Formulations containing 2 mol % or 2.5 mol % PEG-DMG showed 9 nm of particle size growth during TFF, similar to the trend observed with formulations containing 3 mol or 5 mol % PEG-DMG. After nebulization, smaller particle sizes were observed with samples containing the higher mol % PEG-DMG. This trend was consistent with the observation from the previous DoE study (Example 40), as well as the comparison of formulations containing 3 mol % or 5 mol % PEG-DMG.

TABLE 17
Characterization of formulations prepared with 2 mol % or 2.5 mol %
PEG-DMG and tdTomato mRNA.

| | | | | | | | | | | After filtration with | | | |
|---|---|---|---|---|---|---|---|---|---|---|---|---|---|
| | | | | | | | | After | | 0.2 μm membrane | | | |
| Lipid composition (mol %) | | | | | After dilution | | | TFF | | | | | mRNA |
| | | | | PEG- | PS | | Encap | PS | | PS | | Encap | purity |
| DOTAP | ATX12 | DSPC | CHOL | DMG | (nm) | PDI | (%) | (nm) | PDI | (nm) | PDI | (%) | (%) |
| 25 | 25 | 10 | 38 | 2 | 64.92 | 0.113 | 98.2 | 73.52 | 0.158 | 69.88 | 0.079 | 98.8 | NA |
| 25 | 25 | 10 | 37.5 | 2.5 | 61.24 | 0.148 | 96.2 | 70.54 | 0.201 | 67.02 | 0.098 | 97.8 | 88 |

TABLE 18
Characterization of pre- and post-nebulized formulations prepared
with 1.5 mol %, 2 mol % or 2.5% PEG-DMG and tdTomato mRNA.

| | | | | | 1 F/T | | | | Post-neb (0.6 mg/mL) | | | |
|---|---|---|---|---|---|---|---|---|---|---|---|---|
| Lipid composition (mol %) | | | | | | | | mRNA | | | | mRNA |
| | | | | PEG- | PS | | Encap | purity | PS | | Encap | purity |
| DOTAP | ATX12 | DSPC | CHOL | DMG | (nm) | PDI | (%) | (%) | (nm) | PDI | (%) | (%) |
| 25 | 25 | 10 | 38.5 | 1.5 | 74.15 | 0.055 | 99.7 | NA | 543.8 | 0.57 | 98 | 87 |
| 25 | 25 | 10 | 38 | 2 | 70.42 | 0.089 | 99.4 | 90 | 436.7 | 0.584 | 97.8 | 85 |
| 25 | 25 | 10 | 37.5 | 2.5 | 71.38 | 0.086 | 98.8 | 90 | 289.9 | 0.523 | 97.8 | 87 |

When testing transfection efficiency in vitro (FIG. 51E), no difference was observed between the cells treated with formulations containing 1.5 mol % to 2.5 mol % PEG-DMG in pre or post-nebulized forms. Pre-nebulized formulations containing 2 mol % or 2.5 mol % PEG-DMG showed protein expression in lung airway epithelial cells in mice after intratracheal administration (FIG. 51F), but the intensity appeared to be lower than that observed with pre-nebulized formulation containing 1.5 mol % PEG-DMG. Little protein expression was observed in the lungs of mice that received post-nebulized formulations containing either 2 mol % or 2.5 mol % PEG-DMG. This confirms that, among the formulations tested, DOTAP:ATX12:DSPC:CHOL:PEG2000-DMG in a molar ratio of 25:25:10:38.5:1.5 is an optimal lipid composition for efficient mRNA delivery in in vitro and in vivo models.

### Example 43: Initial Buffer Screening for Formulation Stability Under Nebulization and −70° C. Storage Conditions

As noted in Example 38, in addition to mRNA and lipid components, the lipid nanoparticle formulations can also include “storage buffers” containing a variety of excipients. However, changes in buffer pH and/or components can affect the stability of the formulations during manufacturing and/or storage. Additionally, in view of the intention to deliver the mRNA drugs to the lung via inhalation using nebulizers, changes in the buffer components could potentially affect formulation physicochemical properties, such as surface tension and viscosity, and thus affect aerosol droplet size and formulation quality after nebulization.

In this example, lipid compositions containing DOTAP:ATX-012:DSPC:cholesterol:PEG2000-DMG at a molar ratio of 25:25:10:38.5:1.5 were formulated with uncapped hCFTR mRNA at a target N/P 3 and final mRNA concentration of 0.5 mg/mL. These formulations, which contained varying concentrations of glycerol, sucrose, NaCl and HEPES, were tested for particle size (PS), polydispersity index (PDI) and encapsulation efficiency after storage, nebulization, or multiple freeze-thaw cycles (3 F/T) cycles. Post-nebulized formulations were prepared by nebulization of 1:1 WFI diluted 1 F/T formulations (target mRNA concentration 0.25 mg/mL) using vibrating mesh as described in Example 24.

First, the percent sucrose in the storage buffer was varied while fixing the remaining components as pH 8, 50 mM HEPES, 50 mM NaCl, and glycerol 5% (w/v). The formulations were prepared as in the Example 38, with the following exceptions: 1) 2^{nd} dilution and diafiltration buffer was prepared with 50 mM HEPES, 50 mM NaCl and 0% sucrose; 2) 5% (w/v) glycerol and 0% to 15% (w/v) sucrose were added as cryoprotectants to the bulk formulation prior to −70° C. storage.

The results are shown in Table 19. Formulations containing no (0%) sucrose exhibited a substantial particle size increase after −70° C. storage. After 3 F/T, formulations containing 15% (w/v) sucrose showed the smallest particle size growth, indicating that higher percentages of sucrose enhance cryoprotection. Although not as good as 15% (w/v) sucrose, formulations with 5% or 9% (w/v) sucrose also showed relatively small particle size growth after 3 F/T. No encapsulation efficiency differences were observed between formulations with different percentages of sucrose pre- or post-nebulization.

TABLE 19
Characterization of formulations with different percentages of sucrose in the storage buffer.

| Sucrose | Bulk | | | After 1 F/T | | | After 2 F/T | | | After 3 F/T | | | Post-nebulization | | |
|---|---|---|---|---|---|---|---|---|---|---|---|---|---|---|---|
| (%, | PS | | Encap | PS | | Encap | PS | | Encap | PS | | Encap | PS | | Encap |
| w/v) | (nm) | PDI | (%) | (nm) | PDI | (%) | (nm) | PDI | (%) | (nm) | PDI | (%) | (nm) | PDI | (%) |
| 0 | 91.37 | 0.09 | 96.15 | 103 | 0.13 | 97.89 | 116 | 0.176 | 97.92 | 120.5 | 0.205 | 97.76 | 420.0 | 0.54 | 94.9 |
| 5 | | | | 95.72 | 0.099 | 97.72 | 94.55 | 0.112 | 97.83 | 96.42 | 0.086 | 97.76 | 369.2 | 0.53 | 94.1 |
| 9 | | | | 95.78 | 0.116 | 97.76 | 95.99 | 0.094 | 97.92 | 94.87 | 0.083 | 97.82 | 411.9 | 0.57 | 94.5 |
| 15 | | | | 98.29 | 0.15 | 97.82 | 93.74 | 0.14 | 97.94 | 93.54 | 0.11 | 97.84 | 484.9 | 0.61 | 94.7 |

Next, different percentages of glycerol in the storage buffer were tested, while keeping other components fixed at pH 8, 50 mM HEPES, 50 mM NaCl and 9% (w/v) sucrose. The formulations were prepared as in the Example 38, with the following exceptions: 1) 2^{nd} dilution and diafiltration buffer were prepared as 50 mM HEPES, 50 mM NaCl and 0% Sucrose; 2) 9% (w/v) sucrose and 0% to 5% (w/v) glycerol were added as cryoprotectants to the bulk formulation prior to −70° C. storage.

The results are shown in Table 20. Formulations containing no (0%) glycerol exhibited a substantial particle size increase after −70° C. storage. After 3 F/T, formulations containing 50% (w/v) glycerol showed the smallest particle size growth, indicating that higher percentages of glycerol enhance cryoprotection. Although not as good as 5%, formulations with 2.5% glycerol also exhibited relatively good stability. No encapsulation efficiency differences were observed between formulations with different percentages of glycerol pre- or post-nebulization.

TABLE 20
Characterization of formulations with different percentages of glycerol in the storage buffer.

| | Bulk | | | After 1 F/T | | | After 2 F/T | | | After 3 F/T | | | Post-nebulization | | |
|---|---|---|---|---|---|---|---|---|---|---|---|---|---|---|---|
| Glycerol | PS | | Encap | PS | | Encap | PS | | Encap | PS | | Encap | PS | | Encap |
| (%, w/v) | (nm) | PDI | (%) | (nm) | PDI | (%) | (nm) | PDI | (%) | (nm) | PDI | (%) | (nm) | PDI | (%) |
| 0 | 91.37 | 0.09 | 96.15 | 97.62 | 0.107 | 97.94 | 101.3 | 0.136 | 98.01 | 104.2 | 0.144 | 97.80 | 463.4 | 0.64 | 94.6 |
| 2.5 | | | | 94.17 | 0.103 | 97.92 | 94.12 | 0.117 | 97.91 | 94.25 | 0.097 | 98.00 | 517.0 | 0.66 | 94.6 |
| 5 | | | | 95.23 | 0.107 | 98.0 | 92.53 | 0.096 | 97.88 | 91.99 | 0.129 | 97.87 | 491.9 | 0.61 | 93.8 |

The third parameter that was varied was the HEPES concentration, while other components were fixed at pH 8.0, 50 mM NaCl, 9% (w/v) sucrose and 5% (w/v) glycerol. The formulations were prepared as in the Example 38, with the following exceptions: during the TFF process, diafiltration was conducted with buffers composed of 0 mM to 100 mM HEPES, 50 mM NaCl and 0% (w/v) sucrose, with 5% (w/v) glycerol added as cryoprotectant to the bulk formulation prior to −70° C. storage.

The results are shown in Table 21. The HEPES concentration did not affect the particle size or mRNA encapsulation efficiency during 3 F/T cycles. After nebulization, 50 mM and 100 mM HEPES formulations showed slightly smaller particle sizes.

TABLE 21
Characterization of formulations with different concentrations of HEPES in the storage buffer.

| | Bulk | | | After 1 F/T | | | After 2 F/T | | | After 3 F/T | | | Post-nebulization | | |
|---|---|---|---|---|---|---|---|---|---|---|---|---|---|---|---|
| HEPES | PS | | Encap | PS | | Encap | PS | | Encap | PS | | Encap | PS | | Encap |
| (mM) | (nm) | PDI | (%) | (nm) | PDI | (%) | (nm) | PDI | (%) | (nm) | PDI | (%) | (nm) | PDI | (%) |
| 0 | 87.01 | 0.111 | 96.58 | 89.69 | 0.095 | 97.97 | 89.7 | 0.098 | 97.50 | 86.17 | 0.121 | 97.53 | 511.8 | 0.65 | 94.6 |
| 25 | 88.51 | 0.077 | 96.51 | 90.29 | 0.05 | 97.86 | 89.44 | 0.091 | 97.40 | 89.69 | 0.107 | 97.61 | 506.9 | 0.64 | 94.3 |
| 50 | 88.43 | 0.097 | 96.70 | 88.85 | 0.12 | 97.82 | 90.16 | 0.085 | 97.80 | 92.33 | 0.122 | 97.52 | 404.9 | 0.56 | 94.4 |
| 100 | 87.33 | 0.098 | 96.02 | 90.02 | 0.095 | 97.89 | 89.82 | 0.103 | 97.70 | 91.27 | 0.096 | 97.59 | 441.0 | 0.58 | 94.8 |

Lastly, varying concentrations of NaCl in the storage buffer were tested, while keeping other components at pH 8.0, 50 mM HEPES, 9% (w/v) sucrose and 5% (w/v) glycerol. The formulations were prepared as in Example 38, with the following exceptions: 1) 2^{nd} dilution and diafiltration buffer was prepared with 50 mM HEPES, 0 mM NaCl and 9% (w/v) sucrose; 2) 5% (w/v) glycerol and 0 mM to 100 mM NaCl were added to the bulk formulation prior to −70° C. storage.

The results are shown in Table 22. Significant particle size growth was observed with the formulation containing 100 mM NaCl after 3 F/T. No encapsulation efficiency differences were observed in formulations with varying NaCl concentrations pre- or post-nebulization. Smaller post-nebulization particle size was observed with 100 mM NaCl formulation.

TABLE 22
Characterization of formulations with different concentrations of NaCl in the storage buffer.

| | Bulk | | | After 1 F/T | | | After 2 F/T | | | After 3 F/T | | | Post-nebulization | | |
|---|---|---|---|---|---|---|---|---|---|---|---|---|---|---|---|
| NaCl | PS | | Encap | PS | | Encap | PS | | Encap | PS | | Encap | PS | | Encap |
| (mM) | (nm) | PDI | (%) | (nm) | PDI | (%) | (nm) | PDI | (%) | (nm) | PDI | (%) | (nm) | PDI | (%) |
| 0 | 84.39 | 0.092 | 95.74 | 84.54 | 0.113 | 96.51 | 85.68 | 0.089 | 96.40 | 87.71 | 0.095 | 96.81 | 509.0 | 0.65 | 92.6 |
| 25 | | | | 87.19 | 0.087 | 96.79 | 89.63 | 0.106 | 97.00 | 88.96 | 0.111 | 97.00 | 466.8 | 0.61 | 93.3 |
| 50 | | | | 86.76 | 0.102 | 97.02 | 89.99 | 0.113 | 97.00 | 90.5 | 0.109 | 97.44 | 634.0 | 0.75 | 93.2 |
| 100 | | | | 94.06 | 0.128 | 97.17 | 99.46 | 0.114 | 97.40 | 100.2 | 0.121 | 95.97 | 364.0 | 0.49 | 93.5 |

To further optimize the range of each buffer component, formulations were prepared with the buffer components as indicated in Table 23 using design of experiment approach L9 orthogonal array with four factors (A-D, concentration of HEPES, NaCl, sucrose and glycerol, respectively) and three levels for each factor. The analysis method was similar to that described in Example 40. Formulations were prepared with the method described in Example 38 at a target concentration of 0.5 mg/mL uncapped hCFTR mRNA, with the following exceptions: 1) 2^{nd}dilution buffer was fixed as pH 8.0 50 mM HEPES, 50 mM NaCl, 9% (w/v) sucrose; 2) Diafiltration buffer was composed of HEPES, NaCl and sucrose, with the concentrations indicated in Table 23 but no glycerol; 3) 1% to 3% (w/v) glycerol was added as cryoprotectant to bulk formulations prior to −70° C. storage.

The results of particle size, PDI, mRNA encapsulation efficiency and mRNA purity after 1 F/T and post-nebulization are shown in the Table 23. All formulations showed encapsulation efficiency >90%, and no significant decrease was observed in the corresponding post-nebulized formulations.

TABLE 23
DoE orthogonal array buffer optimization and formulation characterization.

| | | | | | | | | | | | | | After 3 F/T |
|---|---|---|---|---|---|---|---|---|---|---|---|---|---|
| | Buffer component | | | | After 1 F/T | | | | Post-nebulization | | | | Particle Size |
| Exper- | Factor A: | Factor B: | Factor C: | Factor D: | | | | mRNA | | | | mRNA | increase |
| iment | HEPES | NaCl | Sucrose | Glycerol | PS | | Encap | purity | PS | | Encap | purity | compared |
| # | (mM) | (mM) | (%) | (%) | (nm) | PDI | (%) | (%) | (nm) | PDI | (%) | (%) | to bulk (nm) |
| 1 | 50 | 10 | 2 | 1 | 95.03 | 0.083 | 95.70 | 80.5 | 552.40 | 0.69 | 93.63 | 73.95 | 11.71 |
| 2 | 50 | 30 | 5 | 2 | 91.17 | 0.118 | 97.24 | 84.62 | 615.70 | 0.75 | 94.70 | 80.52 | 9.14 |
| 3 | 50 | 50 | 9 | 3 | 94.99 | 0.117 | 95.50 | 81.01 | 452.10 | 0.68 | 94.00 | 70.15 | 4.44 |
| 4 | 75 | 10 | 5 | 3 | 90.72 | 0.116 | 95.10 | 80.38 | 846.10 | 0.95 | 92.54 | 71.39 | 2.48 |
| 5 | 75 | 30 | 9 | 1 | 91.44 | 0.086 | 96.47 | 80.96 | 463.00 | 0.69 | 95.18 | 71.74 | 2.31 |
| 6 | 75 | 50 | 2 | 2 | 102.2 | 0.144 | 96.80 | 81.17 | 493.50 | 0.63 | 95.06 | 73.86 | 26.93 |
| 7 | 100 | 10 | 9 | 2 | 89.9 | 0.097 | 96.59 | 81.18 | 668.90 | 0.84 | 94.67 | 73.83 | −0.76 |
| 8 | 100 | 30 | 2 | 3 | 95.25 | 0.097 | 97.17 | 83.78 | 487.60 | 0.63 | 95.09 | 79.2 | 14.30 |
| 9 | 100 | 50 | 5 | 1 | 98.3 | 0.114 | 97.17 | 82.13 | 410.70 | 0.65 | 94.57 | 80.98 | 20.39 |

Results of orthogonal array DoE data are summarized in Tables 24 to 27.

As shown in Table 24, smaller particle size was observed with higher concentrations of sucrose or lower concentrations of NaCl in 1 F/T samples. However, slightly smaller post-nebulization particle sizes were observed in samples containing higher concentrations of NaCl. When the formulations were subjected to 3 F/T cycles, less particle size growth was observed with formulations with lower concentrations of NaCl, higher concentrations of sucrose and higher concentrations of glycerol.

As shown in Table 25, no PDI difference was observed at different levels of each factor in 1 F/T samples. Larger PDI was observed in all post-nebulized samples compared to 1 F/T samples.

As shown in Table 27, no mRNA purity difference was observed at different levels of each factor in 1 F/T samples. Formulations composed of 50 mM or 100 mM HEPES, 30 mM NaCl and 2% to 5% (w/v) sucrose showed slightly better mRNA purity.

TABLE 24
Particle size analysis by orthogonal array design experiment

| | | HEPES | NaCl | Sucrose | Glycerol |
|---|---|---|---|---|---|
| Particle Size (nm) | | (mM) | (mM) | (%, w/v) | (%, w/v) |
| 1 F/T | K1 | 93.73 | 91.88 | 97.49 | 94.92 |
| | K2 | 94.79 | 92.62 | 93.40 | 94.42 |
| | K3 | 94.48 | 98.50 | 92.11 | 93.65 |
| | Range | 1.06 | 6.61 | 5.38 | 1.27 |
| | Ranking | 4 | 1 | 2 | 3 |
| | Preference | 50-100 | 10-30 | 5-9 | 1-3 |
| Post-neb | K1 | 540.07 | 689.13 | 511.17 | 475.37 |
| | K2 | 600.87 | 522.10 | 624.17 | 592.70 |
| | K3 | 522.40 | 452.10 | 528.00 | 595.27 |
| | Range | 78.47 | 237.03 | 113.00 | 117.33 |
| | Ranking | 4 | 1 | 3 | 2 |
| | Preference | 50 or 100 | 50 | 1 | 1 |
| Particle | K1 | 8.43 | 4.48 | 17.65 | 11.47 |
| size | K2 | 10.57 | 8.58 | 10.67 | 11.77 |
| growth | K3 | 11.31 | 17.25 | 2.00 | 7.07 |
| after 3 F/T | Range | 2.88 | 12.78 | 15.65 | 4.70 |
| compared | Ranking | 4 | 2 | 1 | 3 |
| to bulk | Preference | 50 | 10 | 9 | 3 |

TABLE 25
PDI analysis by orthogonal array design experiment.

| | | HEPES | NaCl | Sucrose | Glycerol |
|---|---|---|---|---|---|
| PDI | | (mM) | (mM) | (%, w/v) | (%, w/v) |
| 1 F/T | K1 | 0.11 | 0.10 | 0.11 | 0.09 |
| | K2 | 0.12 | 0.10 | 0.12 | 0.12 |
| | K3 | 0.10 | 0.13 | 0.10 | 0.11 |
| | Range | 0.01 | 0.02 | 0.02 | 0.03 |
| | Ranking | 3 | 2 | 2 | 1 |
| | Preference | No preference | | | |
| Post-neb | KI | 0.71 | 0.83 | 0.65 | 0.68 |
| | K2 | 0.76 | 0.69 | 0.78 | 0.74 |
| | K3 | 0.71 | 0.65 | 0.73 | 0.75 |
| | Range | 0.05 | 0.17 | 0.13 | 0.07 |
| | Ranking | 4 | 1 | 2 | 3 |
| | Preference | 50-100 | 30-50 | 2 | 1 |

TABLE 26
Encapsulation efficiency analysis by
orthogonal array design experiment.

| Encapsulation | | HEPES | NaCl | Sucrose | Glycerol |
|---|---|---|---|---|---|
| efficiency (%) | | (mM) | (mM) | (%, w/v) | (%, w/v) |
| 1 F/T | K1 | 96.15 | 95.80 | 96.56 | 96.45 |
| | K2 | 96.12 | 96.96 | 96.50 | 96.88 |
| | K3 | 96.98 | 96.49 | 96.19 | 95.92 |
| | Range | 0.85 | 0.47 | 0.37 | 0.95 |
| | Ranking | Encapsulation is good in all ranges; no | | | |
| | | preferred ranking | | | |
| | Preference | No preference | | | |
| Post-neb | K1 | 94.11 | 93.61 | 94.59 | 94.46 |
| | K2 | 94.26 | 94.99 | 93.94 | 94.81 |
| | K3 | 94.78 | 94.54 | 94.62 | 93.88 |
| | Range | 0.67 | 1.38 | 0.68 | 0.93 |

| | |
|---|---|
| Ranking | Encapsulation is good in all ranges; no |
| | preferred ranking |
| Preference | No preference |

TABLE 27
mRNA purity analysis by orthogonal array design experiment.

| | | HEPES | NaCl | Sucrose | Glycerol |
|---|---|---|---|---|---|
| mRNA purity (%) | | (mM) | (mM) | (%, w/v) | (%, w/v) |
| 1 F/T | K1 | 82.04 | 80.69 | 81.82 | 81.20 |
| | K2 | 80.84 | 83.12 | 82.38 | 82.32 |
| | K3 | 82.36 | 81.44 | 81.05 | 81.72 |
| | Range | 1.53 | 2.43 | 1.33 | 1.13 |
| | Ranking | 2 | 1 | 3 | 3 |
| | Preference | No preference | | | |
| Post-neb | K1 | 74.87 | 73.06 | 75.67 | 75.56 |
| | K2 | 72.33 | 77.15 | 77.63 | 76.07 |
| | K3 | 78.00 | 75.00 | 71.91 | 73.58 |
| | Range | 5.67 | 4.10 | 5.72 | 2.49 |
| | Ranking | 2 | 3 | 1 | 4 |
| | Preference | 50 or 100 | 30 | 2 to 5 | 1 to 2 |

### Example 44: HEPES Buffer Optimization with N/P 4 Formulations

Lipid compositions containing DOTAP:ATX-012:DSPC:cholesterol:PEG2000-DMG at a molar ratio of 25:25:10:38.5:1.5 were formulated with tdTomato mRNA at a target N/P ratio of 4 and final mRNA concentrations of 0.6 mg/mL, 1.2 mg/mL and 2.0 mg/mL, as described in Example 38, with the following exceptions: 1) 2^{nd} dilution buffer was fixed as pH 8.0 50 mM HEPES, 50 mM NaCl, 9% (w/v) sucrose; 2) Diafiltration buffer was composed of 50 mM HEPES, 30 mM or 50 mM NaCl and 5% or 9% (w/v) sucrose, 3) 2% or 5% glycerol (w/v) or 0.05% (w/v) Tween 20 was additionally added as cryoprotectants to the bulk formulation prior to −70° C. storage.

Parameters such as particle size (PS), polydispersity index (PDI), pH, osmolarity (measured by freezing point depression osmomenter), encapsulation efficiency and mRNA purity were characterized after storage under a variety of conditions, nebulization, or multiple freeze-thaw cycles (3 F/T). Post-nebulized formulations were prepared by nebulization of 1:1 WFI diluted 1 F/T formulations or as it is (no dilution) using an Aerogen Solo vibrating mesh nebulizer as described in Example 26. In this experiment, nebulization outputs of the test formulations were compared to the nebulization outputs of aqueous solution containing 0.9% saline, which was referred as the formulation/buffer nebulization output ratio. This approach allowed for the comparison of the nebulizer output between formulations, while minimizing any variability arising from the nebulizer mesh itself.

In all tested buffer conditions shown in Table 28, encapsulation efficiency was well maintained after 3 F/T as well as after 4 weeks storage at −70° C. Also, no mRNA purity change was observed after 1 month storage at −70° C. The largest particles size growth after 3 F/T occurred in samples in storage buffer containing 50 mM HEPES, 30 mM NaCl, 9% (w/v) sucrose and 0.05% (w/v) Tween 20, which exhibited about 3 nm growth compared to the pre-storage condition. Otherwise, particle size was stable after 3 F/T in all tested conditions. Lower percentages of sucrose or glycerol in the final buffer decreased the osmolarity of the formulation. Since physiological osmolarity is between 275-295 mOsmol/kg, post-nebulized formulations with osmolarity values closest to this range are preferred.

TABLE 28
Characterization of formulations prepared with different storage buffer components.

| | | | | | | | | | | 4-week Storage at −70° C. | | | |
|---|---|---|---|---|---|---|---|---|---|---|---|---|---|
| | | After 1 F/T | | | | | | | | (1 F/T) | | | |
| Formulation | | | Osmolarity | mRNA | | | After 3 F/T | | | | | | mRNA |
| Storage | mRNA | Encap | (mOsmol/ | purity | PS | | Encap | PS | | Encap | PS | | purity |
| Buffer* | (mg/mL) | (%) | kg) | (%) | (nm) | PDI | (%) | (nm) | PDI | (%) | (nm) | PDI | (%) |
| 50/50/9/5G | 0.6 | 99.7 | 1248 | N/A | 74.08 | 0.08 | 99.5 | 75.31 | 0.096 | 99.5 | 72.81 | 0.088 | 88 |
| | 1.2 | 99.7 | 1394 | N/A | 74.15 | 0.055 | 99.5 | 75.55 | 0.072 | 99.4 | 72.31 | 0.072 | 88 |
| | 2.0 | 99.6 | 1298 | 87 | 74.55 | 0.054 | 99.5 | 75.06 | 0.08 | 99.5 | 73.94 | 0.07 | 88 |
| 50/50/9/2G | 0.6 | 99.6 | 878 | 90 | 75.11 | 0.079 | 99.5 | 74.97 | 0.095 | 99.5 | 73.58 | 0.073 | 87 |
| | 1.2 | 99.7 | 869 | N/A | 75.9 | 0.092 | 99.6 | 75.84 | 0.07 | 99.6 | 74.98 | 0.04 | 90 |
| | 2.0 | 99.6 | 891 | 88 | 74.69 | 0.087 | 99.5 | 75.46 | 0.054 | 99.5 | 73.94 | 0.081 | 87 |
| 50/30/5/5G | 0.6 | 99.6 | 1148 | 91 | 74.18 | 0.106 | 99.5 | 74.02 | 0.085 | 99.4 | 73.4 | 0.08 | 90 |
| | 1.2 | 99.8 | 1196 | N/A | 73.97 | 0.085 | 99.4 | 75.95 | 0.066 | 99.4 | 72.72 | 0.076 | 89 |
| | 2.0 | 99.8 | 1186 | 90 | 73.5 | 0.083 | 99.4 | 74.81 | 0.073 | 99.3 | 74.71 | 0.074 | 89 |
| 50/30/5/2G | 0.6 | 99.8 | 723 | 91 | 74 | 0.079 | 99.5 | 73.91 | 0.068 | 99.4 | 72.68 | 0.073 | 86 |
| | 1.2 | 99.4 | 751 | N/A | 75.29 | 0.075 | 99.5 | 74.58 | 0.072 | 99.5 | 75.02 | 0.088 | 90 |
| | 2.0 | 99.8 | 731 | 89 | 74.97 | 0.08 | 99.5 | 74.51 | 0.089 | 99.5 | 72.34 | 0.093 | 89 |
| 50/30/9/5G | 0.6 | 100.1 | 1340 | 87 | 75.52 | 0.106 | 99.4 | 76.06 | 0.087 | 99.6 | 74.8 | 0.073 | 89 |
| | 1.2 | 100.1 | 1330 | N/A | 75.23 | 0.095 | 99.4 | 75.67 | 0.079 | 99.4 | 75.41 | 0.065 | 88 |
| | 2.0 | 100.1 | 1344 | 89 | 76.41 | 0.041 | 99.4 | 75.07 | 0.082 | 99.5 | 74.11 | 0.054 | 90 |
| 50/30/9/2G | 0.6 | 100.2 | 864 | 87 | 75.89 | 0.069 | 99.4 | 76.42 | 0.09 | 99.4 | 74.38 | 0.087 | 90 |
| | 1.2 | 100.1 | 860 | N/A | 75.94 | 0.072 | 99.4 | 76.56 | 0.028 | 99.4 | 74.64 | 0.061 | 90 |
| | 2.0 | 100.2 | 864 | 90 | 76.96 | 0.077 | 99.4 | 76.77 | 0.077 | 99.4 | 73.33 | 0.074 | 90 |
| 50/30/9/1G | 0.6 | 100.2 | 695 | 90 | 75.52 | 0.102 | 99.4 | 77.25 | 0.07 | 99.4 | 74.26 | 0.066 | 90 |
| | 1.2 | 100.2 | 721 | N/A | 75.25 | 0.08 | 99.4 | 73.78 | 0.059 | 99.5 | 75.4 | 0.049 | 89 |
| | 2.0 | 100.2 | 733 | 89 | 75.62 | 0.1 | 99.4 | 76.01 | 0.085 | 99.4 | 74.59 | 0.076 | 89 |
| 50/30/9/ | 0.6 | 99.8 | 548 | 90 | 75.81 | 0.125 | 99.5 | 78.03 | 0.065 | 99.5 | 72.33 | 0.102 | NA |
| 0.05T20 | 1.2 | 99.8 | 583 | N/A | 75.68 | 0.067 | 99.5 | 78.11 | 0.043 | 99.5 | 75.6 | 0.05 | 89 |
| | 2.0 | 100.2 | 548 | 90 | 77.5 | 0.079 | 99.5 | 77.14 | 0.091 | 99.5 | 74.15 | 0.096 | 89 |

*50/50/9/nG refers to 50 mM HEPES, 50 mM NaCl, 9% (w/v) sucrose and n % (w/v) glycerol;
0.05T20 refers to 0.05% (w/v) Tween20.

As shown in Table 29, upon nebulization, encapsulation efficiency and mRNA purity were well maintained in all the samples. At the same formulation concentration at nebulization, no dilution samples (0.6 mg/mL with no dilution) showed smaller particle size than 1:1 WFI diluted samples (0.6 mg/mL after 1:1 WFI dilution). At the same buffer condition, formulations nebulized at higher concentration showed smaller particle size. Overall, the nebulizer output ratio of formulation/saline is much slower in no dilution samples. Among them, samples having a concentration of 2.0 mg/mL showed relatively lower output than samples at lower concentrations.

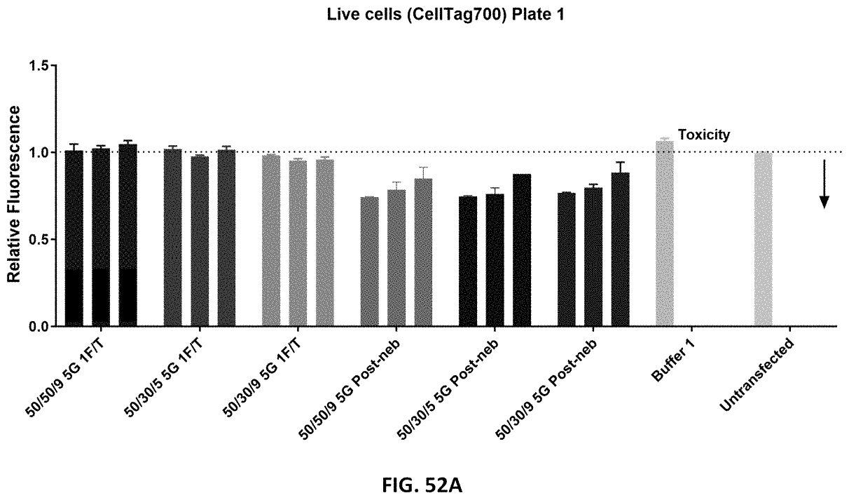

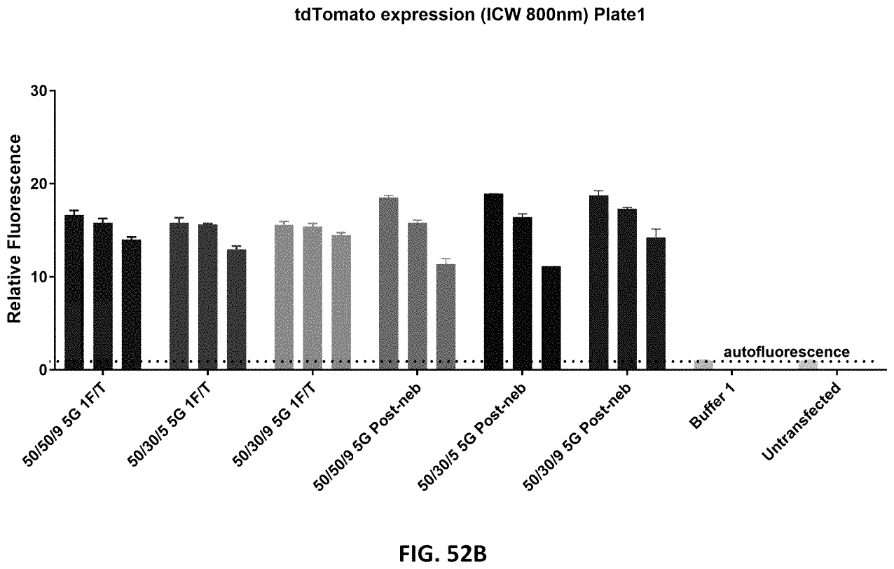

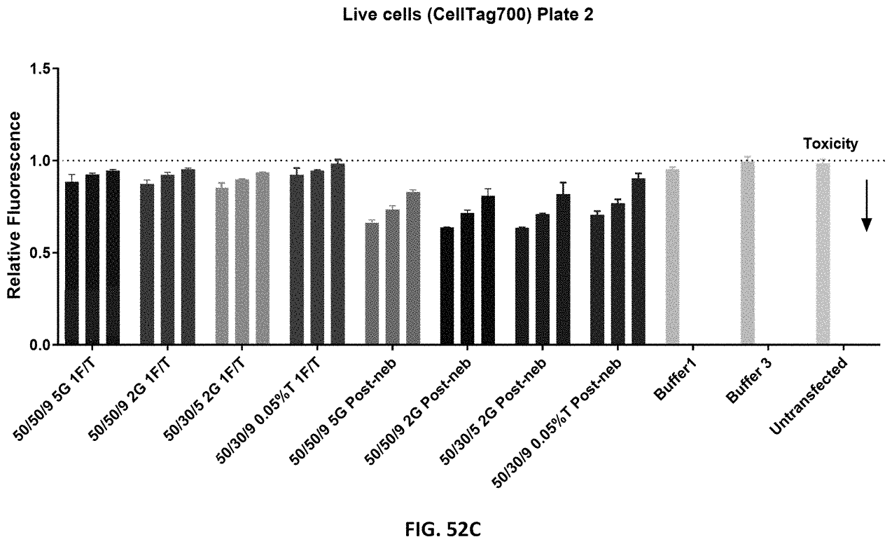

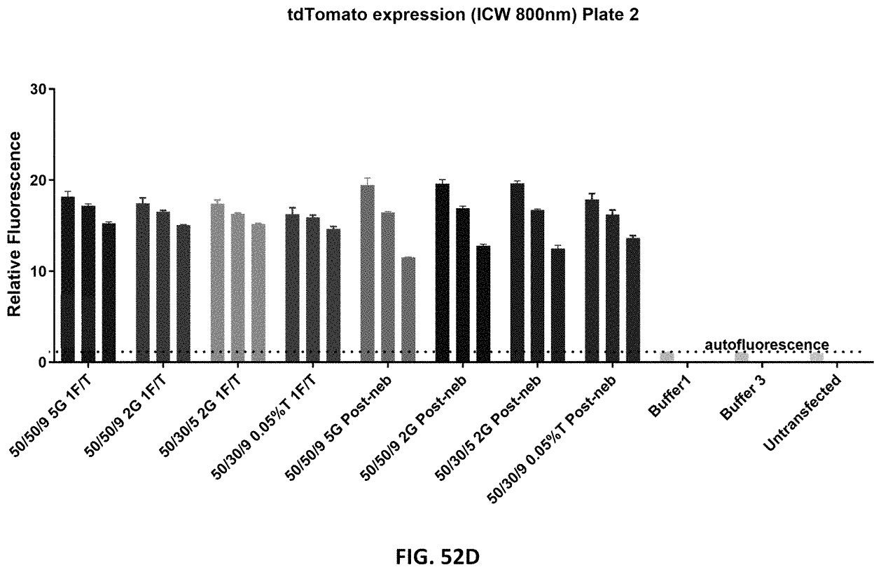

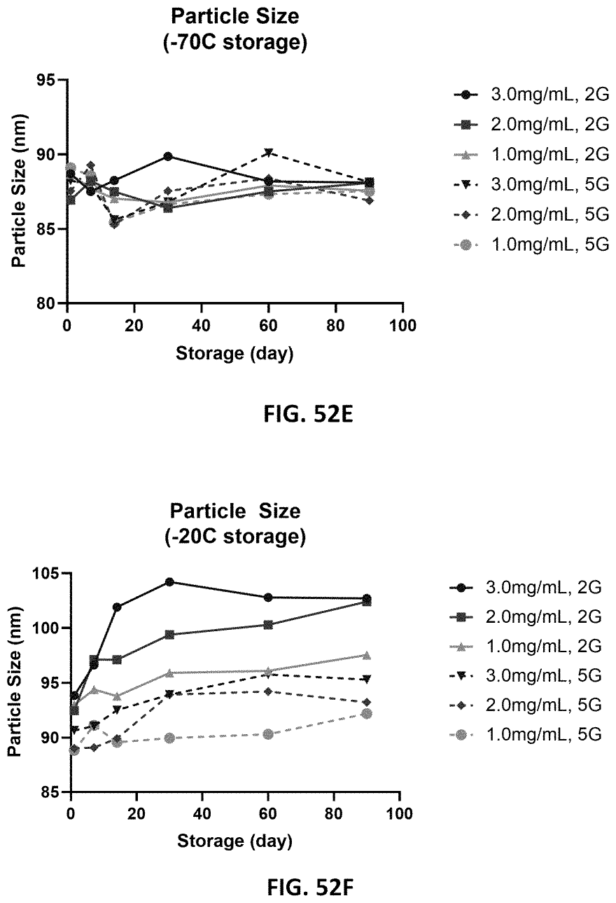

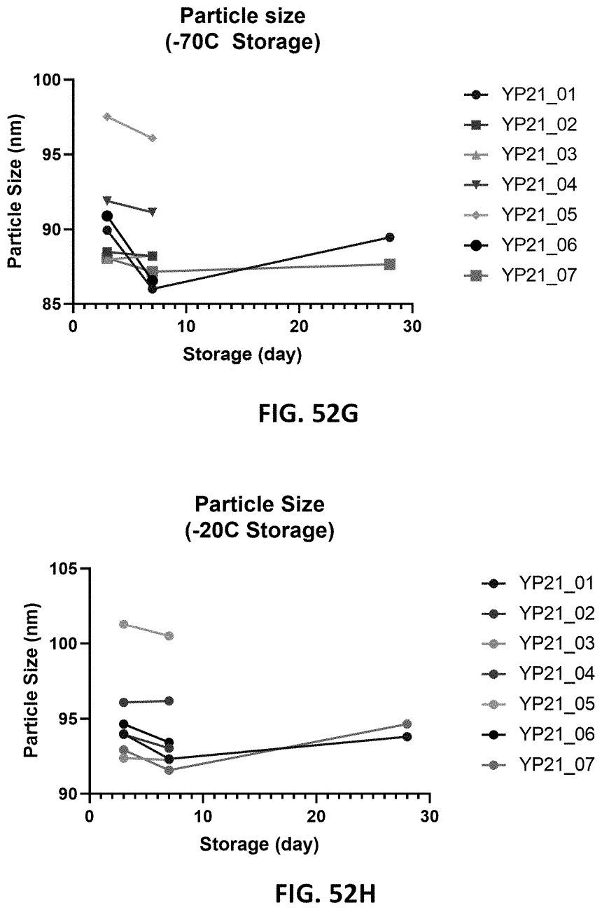

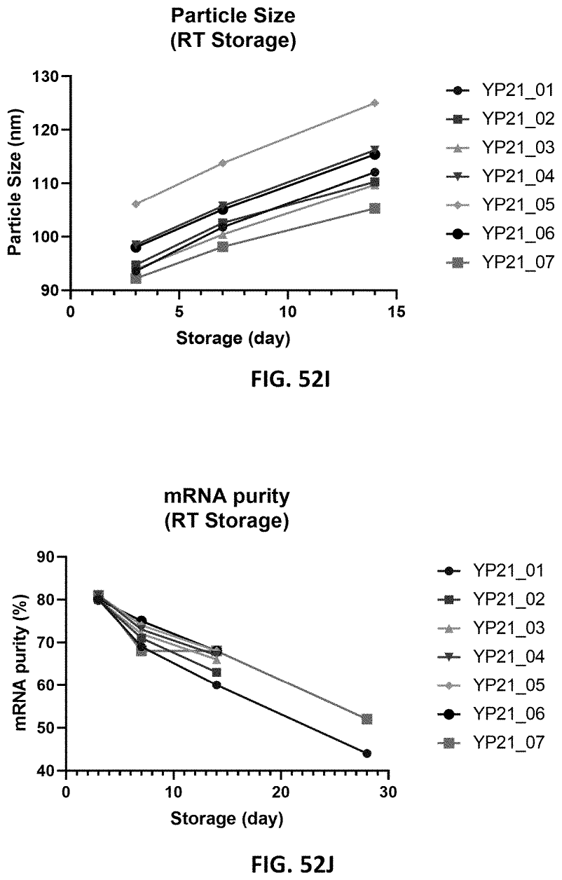

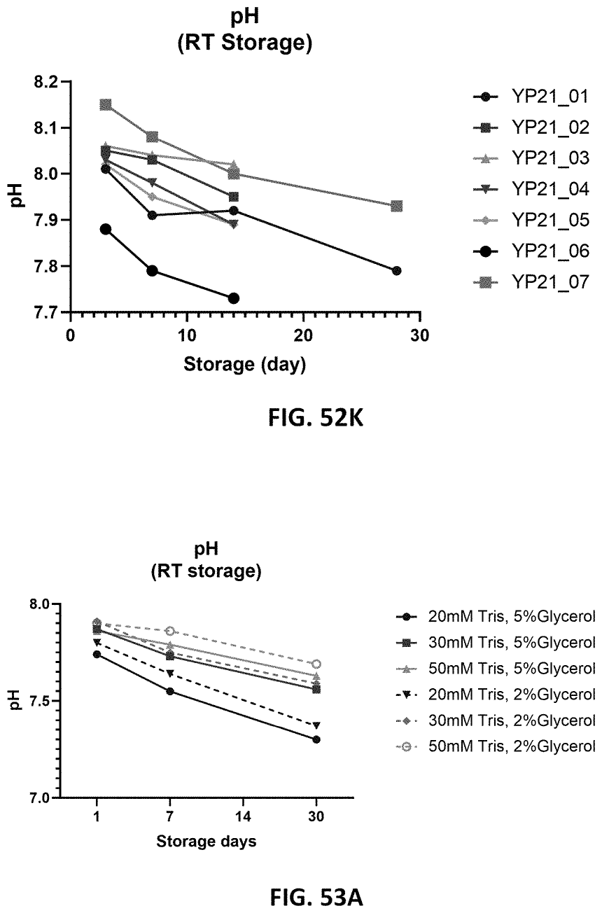

To compare the formulations for transfection efficiency, CFBE cells were treated at three different doses (200 ng, 100 ng and 50 ng) of the samples formulated with final storage buffer containing pH 8.0 50 mM HEPES and 5% (w/v) glycerol with varying concentrations of NaCl (30 mM or 50 mM), sucrose (50% or 90% (w/v)) or cryoprotectant (1%, 2% or 5% (w/v) glycerol, or 0.05% TWEEN20®). In-Cell Western assay was conducted as described in Example 1. As shown in FIGS. 52B and D, protein expression in CFBE cells was similar in both pre- and post-nebulized samples regardless of buffer composition in the test samples.

TABLE 29
Characterization of formulations post-nebulization.

| | Post-nebulization (1:1 WFI) | | | | | | Post-nebulization (no dilution) | | | | | |
|---|---|---|---|---|---|---|---|---|---|---|---|---|
| | | | | | | Nebulizer | | | | | | Nebulizer |
| | | | mRNA | | | output ratio % | | | mRNA | | | output ratio % |
| Storage | mRNA | Encap | purity | PS | | (formulation/ | mRNA | Encap | purity | PS | | (formulation/ |
| Buffer | (mg/mL) | (%) | (%) | (nm) | PDI | saline) | (mg/mL) | (%) | (%) | (nm) | PDI | saline) |
| 50/50/9/5G | 0.3 | 97.7 | 86 | 793.2 | 0.70 | 63.0 | 0.6 | 97.8 | 82 | 334.2 | 0.73 | 37.8 |
| | 0.6 | 98 | 87 | 543.8 | 0.57 | 54.7 | 1.2 | 97.8 | 82 | 261.6 | 0.72 | 31.5 |
| | 1.0 | 98.3 | 88 | 311.7 | 0.78 | 62.2 | 2.0 | 98.3 | 87 | 235.3 | 0.46 | 34.7 |
| 50/50/9/2G | 0.3 | 97.4 | 85 | 799.9 | 0.81 | 71.6 | 0.6 | 98 | 86 | 271.4 | 0.67 | 49.3 |
| | 0.6 | 98 | 87 | 307.5 | 0.94 | 71.6 | 1.2 | 97.7 | 85 | 250.6 | 0.66 | 52.6 |
| | 1.0 | 98.2 | 87 | 330.8 | 0.81 | 49.5 | 2.0 | 97.7 | 85 | 252.1 | 0.48 | 39.5 |
| 50/30/5/5G | 0.3 | 97.2 | 86 | 544.3 | 0.77 | 74.0 | 0.6 | 97.6 | 86 | 348.9 | 0.94 | 56.1 |
| | 0.6 | 97.6 | 87 | 405.3 | 0.91 | 69.3 | 1.2 | 98.1 | 87 | 263.8 | 0.73 | 54.5 |
| | 1.0 | 97.9 | 88 | 355.3 | 0.62 | 62.2 | 2.0 | 97.9 | 82 | 240.6 | 0.76 | 20.9 |
| 50/30/5/2G | 0.3 | 97.3 | 88 | 765.0 | 0.61 | 78.1 | 0.6 | 97.8 | 88 | 292.6 | 1.00 | 62.2 |
| | 0.6 | 97.8 | 86 | 365.6 | 0.99 | 69.5 | 1.2 | 98.1 | 87 | 266.8 | 0.73 | 46.0 |
| | 1.0 | 97.8 | 87 | 317.0 | 0.97 | 66.7 | 2.0 | 98.3 | 86 | 212.5 | 0.45 | 55.7 |
| 50/30/9/5G | 0.3 | 97.5 | 82 | 627.9 | 0.72 | 88.9 | 0.6 | 98.2 | 86 | 381.7 | 0.60 | 43.3 |
| | 0.6 | 97.5 | 81 | 371.2 | 0.65 | 68.3 | 1.2 | 97.9 | 84 | 258.8 | 0.76 | 35.2 |
| | 1.0 | 98.3 | 86 | 259.0 | 0.94 | 71.2 | 2.0 | 98 | 83 | 282.9 | 0.56 | 34.7 |
| 50/30/9/2G | 0.3 | 97.4 | 88 | 751.5 | 0.67 | 63.5 | 0.6 | 97.4 | 89 | 437.8 | 0.59 | 45.4 |
| | 0.6 | 97.8 | 82 | 546.0 | 0.61 | 63.8 | 1.2 | 97.8 | 85 | 284.2 | 0.73 | 38.3 |
| | 1.0 | 97.6 | 84 | 366.2 | 1.00 | 46.2 | 2.0 | 97.8 | 86 | 259.6 | 0.49 | 33.9 |
| 50/30/9/1G | 0.3 | 97.4 | 87 | 483.8 | 0.65 | 86.7 | 0.6 | 97.7 | 87 | 290.2 | 0.75 | 75.0 |
| | 0.6 | 97.8 | 92 | 355.5 | 0.76 | 76.6 | 1.2 | 98.1 | 87 | 242.2 | 0.69 | 56.4 |
| | 1.0 | 98.2 | 89 | 231.3 | 0.92 | 72.9 | 2.0 | 97.8 | 87 | 250.2 | 0.49 | 38.8 |
| 50/30/9/0.0 | 0.3 | 97.4 | 87 | 311.3 | 0.82 | 81.1 | 0.6 | 97.2 | 82 | 291.9 | 0.58 | 45.2 |
| 5T | 0.6 | 97.7 | 86 | 337.5 | 0.54 | 87.6 | 1.2 | 97.8 | 85 | 354.5 | 0.69 | 38.2 |
| | 1.0 | 98.2 | 88 | 254.2 | 0.56 | 62.0 | 2.0 | 97.8 | 86 | 255.0 | 0.46 | 30.8 |

*50/50/9/nG refers to 50 mM HEPES, 50 mM NaCl, 9% (w/v) sucrose and n % (w/v) glycerol;
0.05T20 refers to 0.05% (w/v) TWEEN20.

The mRNA-lipid formulations were further studied to determine whether they could be further developed to have acceptable properties for administration by inhalation, as described in Example 24. Droplet size was characterized by Marple Cascade Impactor at a sampling flow rate of 2 L/min. As shown in Table 30, droplet size was within a range of 2.40-3.35 μm in all tested samples, indicating it is within an acceptable range for inhalable droplet size, reported as median mass aerodynamic diameter (MMAD) and its geometric size distribution (GSD). Additionally, 1:1 WFI diluted samples showed smaller droplet size than no diluted samples after nebulization. Also, formulations composed of 30 mM NaCl and 5% (w/v) sucrose showed slightly smaller droplet size than those composed 50 mM NaCl and 9% (w/v) sucrose when other buffer components are the same.

TABLE 30
Formulation droplet size measurement
by Marple Cascade Impactor.

| | | mRNA concentration at | Droplet Size (μm) | |
|---|---|---|---|---|
| Buffer | Dilution? | nebulization (mg/mL) | MMAD | GSD |
| 50/30/5/5G | 1:1 WFI | 0.6 | 2.47 | 1.73 |
| | | 1.0 | 2.78 | 1.94 |
| | None | 0.6 | 2.99 | 1.75 |
| | | 1.2 | 2.80 | 1.63 |
| | | 2.0 | 2.83 | 1.67 |
| 50/30/5/2G | 1:1 WFI | 0.6 | 2.57 | 1.86 |
| | | 1.2 | 2.40 | 1.90 |
| | None | 0.6 | 2.96 | 1.74 |
| | | 1.2 | 2.85 | 1.79 |
| | | 2.0 | 2.74 | 1.65 |
| 50/50/9/5G | 1:1 WFI | 1.0 | 2.71 | 1.90 |
| | None | 1.2 | 3.35 | 1.77 |
| 50/50/9/2G | 1:1 WFI | 0.6 | 2.68 | 1.63 |
| | | 1.0 | 2.80 | 1.72 |
| | None | 0.6 | 3.15 | 1.78 |
| | | 1.2 | 3.24 | 1.93 |
| | | 2.0 | 2.66 | 1.66 |

*50/50/9/nG refers to 50 mM HEPES, 50 mM NaCl, 9% (w/v) sucrose and n % (w/v) glycerol.

Since there was no substantial difference in transfection efficiency or droplet size between the formulations prepared with 50 mM HEPES, 30 mM NaCl, 5% (w/v) sucrose or 50 mM HEPES, 50 mM NaCl, 9% (w/v) sucrose, and that previous results showed that higher percentages of sucrose provided better stability (Example 43), further experiments were designed using formulations prepared as described in Example 38, with the following exceptions: 1) 2^{nd} dilution and diafiltration buffer was prepared with 50 mM HEPES, 50 mM NaCl and 9% (w/v) sucrose; 2) 2% or 5% (w/v) glycerol were added as cryoprotectants to the bulk formulation prior to −70° C. storage; 3) Target mRNA concentration of 1 mg/mL, 2 mg/mL or 3 mg/mL; 4) Stored at −70° C. or −20° C. and monitored for long-term stability up to 3 months.

As was shown in Table 31, all tested formulations showed good encapsulation and mRNA purity after 3 months of storage at both −70° C. and −20° C. No particle size change was observed in samples stored at −70° C. (FIG. 52E), and particle size was similar regardless of percent glycerol or mRNA concentration. However, at −20° C. storage, formulations containing 5% (w/v) glycerol showed less particle size growth than formulations containing 2% (w/v) glycerol. Also, the particle size increase during the −20° C. storage was concentration dependent; higher concentrations of mRNA showed more particle size growth.

TABLE 31
Characterization of formulations after 3 months of storage at −70° C. and −20° C.

| | | | mRNA | | | | | | mRNA |
|---|---|---|---|---|---|---|---|---|---|
| Storage | Storage | Glycerol | conc. | | Osmolarity | PS | | Encap | purity |
| Time | Temp | (%, w/v) | (mg/mL) | pH | (mOsm/kg) | (nm) | PDI | (%) | (%) |
| 1 Day | −70° C. | 2 | 1 | 8 | 812 | 88.67 | 0.11 | 99.8 | 87 |
| | | | 2 | 8.01 | 847 | 86.94 | 0.10 | 99.8 | 88 |
| | | | 3 | 7.99 | 848 | 88.71 | 0.10 | 99.7 | 88 |
| | | 5 | 1 | 8.01 | 1228 | 89.11 | 0.11 | 99.8 | 89 |
| | | | 2 | 8.03 | 1228 | 87.54 | 0.10 | 99.8 | 88 |
| | | | 3 | 8.02 | 1220 | 88.25 | 0.07 | 99.8 | 88 |
| | −20° C. | 2 | 1 | 7.98 | 807 | 92.99 | 0.120 | 99.4 | 89 |
| | | | 2 | 7.98 | 837 | 92.46 | 0.130 | 99.4 | 90 |
| | | | 3 | 7.9 | 835 | 93.85 | 0.112 | 99.1 | 88 |
| | | 5 | 1 | 7.99 | 1260 | 88.84 | 0.113 | 99.2 | 89 |
| | | | 2 | 8.02 | 1254 | 89.02 | 0.134 | 99.3 | 88 |
| | | | 3 | 8 | 1232 | 90.65 | 0.120 | 99.2 | 89 |
| 3 Months | −70° C. | 2 | 1 | 7.95 | 818 | 87.57 | 0.122 | 99.2 | 91 |
| | | | 2 | 7.96 | 843 | 88.1 | 0.117 | 99.3 | 91 |
| | | | 3 | 7.92 | 852 | 88.12 | 0.113 | 99.3 | 86 |
| | | 5 | 1 | 7.94 | 1240 | 87.54 | 0.117 | 99.2 | 87 |
| | | | 2 | 7.93 | 1234 | 86.9 | 0.12 | 99.3 | 88 |
| | | | 3 | 7.93 | 1224 | 88.14 | 0.092 | 99.3 | 90 |
| | −20° C. | 2 | 1 | 7.95 | 830 | 97.54 | 0.222 | 99.2 | 87 |
| | | | 2 | 7.96 | 846 | 102.4 | 0.209 | 99.2 | 86 |
| | | | 3 | 7.95 | 861 | 102.7 | 0.186 | 99.2 | 87 |
| | | 5 | 1 | 7.94 | 1282 | 92.19 | 0.167 | 99.3 | 91 |
| | | | 2 | 7.92 | 1274 | 93.23 | 0.147 | 99.3 | 90 |
| | | | 3 | 7.94 | 1258 | 95.29 | 0.152 | 99.2 | 89 |

Since the presence of 2% (w/v) glycerol in the formulations was insufficient to provide stable particle size during storage at −20° C., and further increases in glycerol concentration were expected to increase formulation osmolality, alternative diafiltration buffers were evaluated, such as different HEPES or Tris concentration or different pH 7.8 to 8.2). The formulations were prepared as described in Example 38, with the following exceptions: 1) 2^{nd} dilution buffer was fixed as pH 8.0 50 mM HEPES, 50 mM NaCl, 9% (w/v) sucrose; 2) Diafiltration buffer was composed of HEPES, NaCl and 9% (w/v) sucrose, with the pH and concentrations indicated in Table 32; 3) 2% (w/v) glycerol was added as cryoprotectant to bulk formulations prior to storage at −70° C. or −20° C. Some samples were also maintained at room temperature (RT) storage to evaluate accelerated degradation conditions on parameters such as particle size, pH and mRNA purity.

As shown in Table 32, when the diafiltration buffer contained 200 mM NaCl, there was more particle size growth during TFF, and larger particle size was observed after 1 day of storage at −70° C. or −20° C. The formulations prepared with pH 8.2 diafiltration buffer showed smaller particle size after storage at −20° C. when compared to the control at day 1 storage (Table 33). The 1-month stability data in FIGS. 52G-I indicated similar particle size after long term storage at −70° C. or −20° C. when compared to controls, but less particle size growth was observed in samples stored at RT. The encapsulation efficiency, mRNA purity and pH were maintained after 1 month storage at −70° C. or −20°. However, samples prepared with pH 8.2 buffer showed less of a decrease in purity compared to controls (FIG. 52J). Formulations with higher concentrations of HEPES showed less of a pH reduction during long-term storage at room temperature (FIG. 52K).

TABLE 32
Characterization of formulations with different buffer composition during processing.

| Diafiltration | After 2^{nd} | | | | | | | | | |
|---|---|---|---|---|---|---|---|---|---|---|
| buffer | Dilution | | | | | | | | | |
| (HEPES | Formulation | | | | | | | | | |
| (mM)/NaCl | Details | | | After TFF | | Bulk Formulation | | | | |
| (mM)/sucrose | PS | | Encap | PS | | | | PS | | Encap |
| (%, w/v)) | (nm) | PDI | (%) | (nm) | PDI | pH | Osmo | (nm) | PDI | (%) |
| HEPES pH8.0 | 80.28 | 0.142 | 98.90 | 82.90 | 0.114 | 8.03 | 522.00 | 81.73 | 0.089 | 99.5 |
| 50/50/9 (control) | | | | | | | | | | |
| HEPES pH8.0 | | | | 80.56 | 0.116 | 8.04 | 634.00 | 82.63 | 0.13 | 99.50 |
| 100/50/9 | | | | | | | | | | |
| HEPES pH8.0 | | | | 83.46 | 0.151 | 8.07 | 888.00 | 82.94 | 0.10 | 99.50 |
| 200/50/9 | | | | | | | | | | |
| HEPES pH8.0 | | | | 83.83 | 0.133 | 8.11 | 617.00 | 82.21 | 0.10 | 99.6 |
| 50/100/9 | | | | | | | | | | |
| HEPES pH8.0 | | | | 86.50 | 0.159 | 8.04 | 816.00 | 84.29 | 0.12 | 99.6 |
| 50/200/9 | | | | | | | | | | |
| HEPES pH7.8 | | | | 83.275 | 0.125 | 7.94 | 513.00 | 83.41 | 0.13 | 99.6 |
| 50/50/9 | | | | | | | | | | |
| HEPES pH8.2 | | | | 83.00 | 0.107 | 8.23 | 547.00 | 84.38 | 0.11 | 99.5 |
| 50/50/9 | | | | | | | | | | |

TABLE 33
Characterization of formulations with different buffer composition after storage at −70° C. or −20° C.

| Formulation | | | | | | | | | | | | |
|---|---|---|---|---|---|---|---|---|---|---|---|---|
| Details | | | | | | | | | | | | |
| Diafiltration | | | | | | | | | | | | |
| buffer (HEPES | −70° C. Storage (Day 1) | | | | | | −20° C. Storage (Day 1) | | | | | |
| (mM)/NaCl | | | | | | mRNA | | | | | | mRNA |
| (mM)/sucrose | | | PS | | Encap | purity | | | PS | | Encap | purity |
| (%, w/v)) | pH | Osmo | (nm) | PDI | (%) | (%) | pH | Osmo | (nm) | PDI | (%) | (%) |
| HEPES pH8.0 | 8.03 | 522 | 81.73 | 0.089 | 99.5 | 90 | 8.08 | 876 | 94.01 | 0.15 | 99.1 | 88 |
| 50/50/9 (control) | | | | | | | | | | | | |
| HEPES pH8.0 | 8.04 | 634 | 82.63 | 0.13 | 99.50 | NA | 8.07 | 1021 | 93.97 | 0.19 | 99.3 | 88 |
| 100/50/9 | | | | | | | | | | | | |
| HEPES pH8.0 | 8.07 | 888 | 82.94 | 0.10 | 99.50 | 90 | 8.09 | 1278 | 92.39 | 0.17 | 99.2 | 86 |
| 200/50/9 | | | | | | | | | | | | |
| HEPES pH8.0 | 8.11 | 617 | 82.21 | 0.10 | 99.6 | 91 | 8.08 | 952 | 96.10 | 0.14 | 99.3 | 84 |
| 50/100/9 | | | | | | | | | | | | |
| HEPES pH8.0 | 8.04 | 816 | 84.29 | 0.12 | 99.6 | 92 | 8.06 | 1157 | 101.30 | 0.21 | 99 | 89 |
| 50/200/9 | | | | | | | | | | | | |
| HEPES pH7.8 | 7.94 | 513 | 83.41 | 0.13 | 99.6 | 90 | 7.92 | 858 | 94.66 | 0.17 | 99.2 | 88 |
| 50/50/9 | | | | | | | | | | | | |
| HEPES pH8.2 | 8.23 | 547 | 84.38 | 0.11 | 99.5 | 90 | 8.26 | 894 | 92.94 | 0.14 | 99.2 | 88 |
| 50/50/9 | | | | | | | | | | | | |

### Example 45: Tris Buffer Optimization

In this example, storage buffers containing different concentrations of Tris were evaluated. Lipid compositions containing DOTAP:ATX-012:DSPC:cholesterol:PEG2000-DMG at a molar ratio of 25:25:10:38.5:1.5 were formulated with capped hCFTR mRNA at a target N/P ratio of 4 and a final mRNA concentration of 3.0 mg/mL, essentially as described in Example 38, with the following exceptions: 1) pH 4.0 5 mM citrate buffer containing 100 mM NaCl was used when mixing lipids with mRNA; 2) 2^{nd} dilution buffer was composed of pH 8.0 50 mM Tris buffer, 50 mM NaCl and 9% (w/v) sucrose; 3) Diafiltration buffer was composed of pH 8.0 20 mM to 50 mM Tris, 50 mM NaCl and 9% (w/v) sucrose as indicated in Table 34; 4) 2% or 5% (w/v) glycerol was added as cryoprotectant to the bulk formulation prior to −70° C., −20° C. or RT storage.

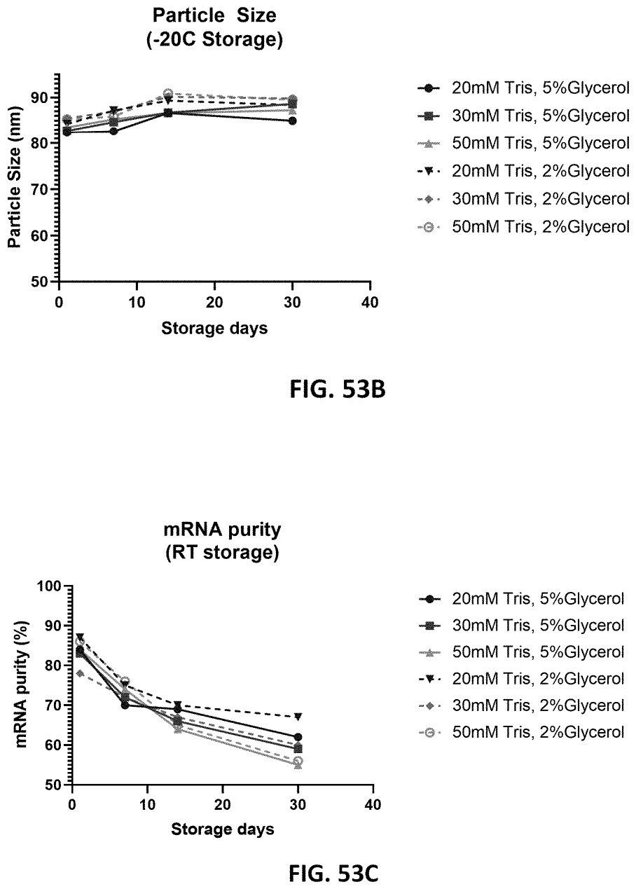

As shown in Table 34, formulations prepared with different concentration of Tris buffer showed similar particle size, good encapsulation efficiency (>99%) and mRNA purity (>85%) through the production process. After 1 month of storage at −70° C. or −20° C., all formulations showed that encapsulation efficiency and mRNA purity were maintained. The particle size was also stable after storage at −70° C. in all tested samples. However, after −20° C. storage, slight particle size growth was observed in all samples, but to a lesser extent in samples containing 5% (w/v) glycerol (FIG. 53B). Overall, particle size was controlled within 95 nm in all samples after 1 month storage at −70° C. or −20° C. The samples were also stored at the room temperature (RT) to assess stability under accelerated degradation conditions. As is shown FIG. 53A, a much higher pH drop was observed with the formulations prepared with 20 mM Tris after one day of storage. Also, at the same Tris concentration, samples containing 5% (w/v) glycerol showed lower pH than the 2% (w/v) glycerol samples. When comparing mRNA purity, lower concentrations of Tris and glycerol showed better mRNA purity (FIG. 53C).

TABLE 34
Characterization of formulations with different concentration of Tris buffer.

| | After 2^{nd} dilution | | | | | | Bulk Formulation | | | | |
|---|---|---|---|---|---|---|---|---|---|---|---|
| Tris | Formulation Details | | | | After TFF | | | | | | mRNA |
| conc. | PS | | Encap | | PS | | | PS | | Encap | purity |
| (mM) | (nm) | PDI | (%) | pH | (nm) | PDI | pH | (nm) | PDI | (%) | (%) |
| 20 | 75.16 | 0.13 | 99 | 7.47 | 78.63 | 0.14 | 7.90 | 79.51 | 0.134 | 99.6 | 92 |
| 30 | | | | | 79.47 | 0.155 | 7.98 | 79.58 | 0.14 | 99.6 | 92 |
| 50 | | | | | 78.74 | 0.15 | 7.93 | 79.29 | 0.14 | 99.6 | 92 |

TABLE 35
Characterization of formulations with different concentrations of Tris
buffer after storage under different conditions.

| | | | Tris | | Osmo | | | | mRNA |
|---|---|---|---|---|---|---|---|---|---|
| Storage | Storage | Glycerol | conc. | | (mOsmo/ | PS | | Encap | purity |
| Time | Temp | (%) | (mM) | pH | kg) | (nm) | PDI | (%) | (%) |
| 1 Day | −70° C. | 5 | 20 | 7.73 | 1170 | 82.22 | 0.189 | 99.9 | 81 |
| | | | 30 | 7.88 | 1164 | 83.21 | 0.149 | 99.9 | 78 |
| | | | 50 | 7.86 | 1204 | 83.73 | 0.168 | 99.9 | 84 |
| | | 2 | 20 | 7.83 | 776 | 83.87 | 0.148 | 99.9 | 81 |
| | | | 30 | 7.95 | 758 | 83.34 | 0.148 | 99.9 | 84 |
| | | | 50 | 7.88 | 832 | 84.16 | 0.144 | 99.9 | 83 |
| | −20° C. | 5 | 20 | 7.78 | 1238 | 82.37 | 0.153 | 99.5 | 83 |
| | | | 30 | 7.89 | 1166 | 82.74 | 0.143 | 99.4 | 83 |
| | | | 50 | 7.89 | 1134 | 83.52 | 0.136 | 99.4 | 86 |
| | | 2 | 20 | 7.84 | 775 | 84.24 | 0.136 | 99.4 | 82 |
| | | | 30 | 7.94 | 790 | 85.51 | 0.167 | 99.4 | 85 |
| | | | 50 | 7.9 | 833 | 85.28 | 0.174 | 99.4 | 86 |
| | RT | 5 | 20 | 7.74 | 1134 | 83.44 | 0.153 | 99.4 | 84 |
| | | | 30 | 7.87 | 1174 | 83.43 | 0.154 | 99.4 | 83 |
| | | | 50 | 7.86 | 1250 | 84.29 | 0.185 | 99.5 | 84 |
| | | 2 | 20 | 7.8 | 780 | 83.54 | 0.117 | 99.4 | 87 |
| | | | 30 | 7.91 | 800 | 84.38 | 0.142 | 99.5 | 78 |
| | | | 50 | 7.9 | 851 | 83.97 | 0.161 | 99.5 | 86 |
| 1 Month | −70° C. | 5 | 20 | 7.83 | 1120 | 83.41 | 0.125 | 99.9 | 86 |
| | | | 30 | 7.97 | 1152 | 83.56 | 0.132 | 99.8 | 83 |
| | | | 50 | 7.94 | 1196 | 80.71 | 0.135 | 99.9 | 86 |
| | | 2 | 20 | 7.89 | 768 | 85.38 | 0.146 | 99.9 | 84 |
| | | | 30 | 7.97 | 798 | 85.28 | 0.161 | 99.8 | 86 |
| | | | 50 | 7.98 | 811 | 83.65 | 0.152 | 99.9 | 86 |
| | −20° C. | 5 | 20 | 7.82 | 1126 | 84.98 | 0.161 | 99.4 | 90 |
| | | | 30 | 7.93 | 1150 | 88.62 | 0.18 | 99.4 | 88 |
| | | | 50 | 7.9 | 1194 | 87.25 | 0.172 | 99.5 | 89 |
| | | 2 | 20 | 7.87 | 773 | 88.38 | 0.191 | 99.4 | 90 |
| | | | 30 | 7.99 | 787 | 89.81 | 0.197 | 99.2 | 89 |
| | | | 50 | 7.96 | 838 | 89.52 | 0.199 | 99.4 | 87 |
| | RT | 5 | 20 | 7.3 | 1150 | 121.6 | 0.246 | 99.3 | 62 |
| | | | 30 | 7.56 | 1154 | 124.5 | 0.272 | 99.0 | 59 |
| | | | 50 | 7.63 | 1212 | 126.9 | 0.249 | 99.0 | 55 |
| | | 2 | 20 | 7.37 | 772 | 122.3 | 0.25 | 99.3 | 67 |
| | | | 30 | 7.59 | 797 | 124.8 | 0.27 | 99.1 | 60 |
| | | | 50 | 7.69 | 834 | 126.4 | 0.261 | 98.9 | 56 |

These formulations were further studied to determine whether they could be further optimized to have acceptable properties for administration by inhalation. Formulations were diluted with WFI either by 1:1 or 1:2 (v/v), as indicated in the Table 36, and nebulized via PARI eflow nebulizers. Post-characterization parameters such as particle size, encapsulation efficiency, mRNA purity, nebulization output and droplet size were evaluated. The droplet size was characterized by Marple Cascade Impactor at a sampling flow rate of 2 L/min. As shown in Table 36, the nebulization output was similar in all tested samples, which was within 0.8-1.0 mL/min. The droplet size was within 3.2-3.7 μm in all tested samples, indicating that it falls within an acceptable inhalable droplet size range (<5 μm). No significant differences were observed in mRNA encapsulation efficiency or mRNA purity among tested samples.

TABLE 36
Post-nebulization and droplet size characterization.

| | | mRNA | | | | | | | Droplet |
|---|---|---|---|---|---|---|---|---|---|
| | | conc. at | Tris | Neb | | | | | Size |
| Glycerol | WFI | nebulization | conc. | output | PS | | Encap | FA | (MMAD ± |
| % | Dilution | (mg/mL) | (mM) | (mL/min) | (nm) | PDI | (%) | (%) | GSD, μm) |
| 2% | 1:1 | 1.5 | 20 | 0.958 | 196.8 | 0.71 | 98.1 | 75 | 3.27 ± 1.75 |
| | | | 30 | 0.914 | 209.3 | 0.76 | 97.8 | 70 | 3.34 ± 1.72 |
| | | | 50 | 0.880 | 221.7 | 0.70 | 98.1 | 79 | 3.72 ± 1.60 |
| 5% | 1.:2 | 1.0 | 20 | 0.819 | 276.8 | 0.47 | 97.9 | 79 | 3.70 ± 1.81 |
| | | | 30 | 1.035 | 289.0 | 0.56 | 98 | 77 | 3.18 ± 1.81 |
| | | | 50 | 0.949 | 247.5 | 0.61 | 97.9 | 81 | 3.18 ± 1.69 |

Since formulations containing 20 mM Tris buffer showed a substantial pH drop during RT storage, formulations containing 30 mM vs 50 mM Tris as the final storage buffer were compared. The formulation production steps were similar to those mentioned previously, with the following exceptions. When 50 mM Tris buffer was used as the diafiltration/storage buffer, 75 mM Tris, 50 mM NaCl and 9% (w/v) sucrose was used as the 2^{nd} dilution buffer. The final mRNA concentration was 3.0 mg/mL.

As is shown in Tables 37 and 38, formulations prepared with different concentrations of Tris buffer showed similar particle size, good encapsulation efficiency (>99%) and mRNA purity (>85%) during the production process. However, when stored at −70° C. or 20° C., larger particle size was observed for samples containing 50 mM Tris. Parameters such as encapsulation efficiency and mRNA purity were similar, and no significant pH drop was observed in the RT samples within 1 day.

TABLE 37
Characterization of formulations prepared with different concentrations of Tris buffer.

| Buffer Details | | | | | | | | Bulk | | | | |
|---|---|---|---|---|---|---|---|---|---|---|---|---|
| Tris conc. in | Tris conc. in | After 2^{nd} dilution | | | | After TFF | | | | | | mRNA |
| 2^{nd} dilution | diafiltration/ | PS | | Encap | | PS | | PS | | | Encap | purity |
| buffer | storage buffer | (nm) | PDI | (%) | pH | (nm) | PDI | (nm) | PDI | pH | (%) | (%) |
| 50 mM | 30 mM | 79.5 | 0.114 | 99.0 | 7.54 | 84.52 | 0.145 | 83.83 | 0.091 | 8.01 | 99.9 | 85 |
| 75 mM | 50 mM | 79.91 | 0.105 | 98.8 | 7.74 | 85.04 | 0.172 | 84.07 | 0.134 | 8.04 | 99.9 | 87 |

TABLE 38
Characterization of formulations with different concentrations of Tris buffer after storage
under different conditions.

| Storage | Storage | Tris conc. in | | PS | | Encap | mRNA |
|---|---|---|---|---|---|---|---|
| Time | Temp | storage buffer | pH | (nm) | PDI | (%) | purity (%) |
| 1 Day | −70° C. | 30 mM | 7.93 | 89.05 | 0.18 | 99.4 | 82 |
| | | 50 mM | 7.99 | 88.31 | 0.163 | 99.5 | 85 |
| | −20° C. | 30 mM | 7.91 | 87.16 | 0.165 | 99.5 | 82 |
| | | 50 mM | 8 | 90.36 | 0.176 | 99.3 | 85 |
| | RT | 30 mM | 7.87 | 89.49 | 0.17 | 99.4 | 85 |
| | | 50 mM | 7.94 | 90.2 | 0.18 | 99.5 | 74 |
| 2 Weeks | −70° C. | 30 mM | 7.85 | 89.24 | 0.151 | 99.4 | 84 |
| | | 50 mM | 7.92 | 90.56 | 0.196 | 99.4 | 83 |
| | −20° C. | 30 mM | 7.84 | 91.17 | 0.178 | 99.4 | 79 |
| | | 50 mM | 7.93 | 96.57 | 0.211 | 99.3 | 87 |
| | RT | 30 mM | 7.62 | 129.7 | 0.379 | 99.3 | 66 |
| | | 50 mM | 7.7 | 123.4 | 0.326 | 99.2 | 64 |
| 1 Month | −70° C. | 30 mM | 7.97 | 85.72 | 0.154 | 99.3 | 85 |
| | | 50 mM | 8 | 89.25 | 0.171 | 99.4 | 86 |
| | −20° C. | 30 mM | 7.94 | 91.98 | 0.163 | 99.4 | 84 |
| | | 50 mM | 7.98 | 96.24 | 0.199 | 99.4 | 84 |

### Further Considerations

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

### Sequences

### Coding Regions

(pARM764)
SEQ ID NO: 1
ATGCAGAGGTCGCCTCTGGAAAAGGCCAGCGTTGTCTCCAAACTTTTTTTCAGCTGGACC
AGACCAATTTTGAGGAAAGGATACAGACAGCGCCTGGAATTGTCAGACATATACCAAATC
CCTTCTGTTGATTCTGCTGACAATCTATCTGAAAAATTGGAAAGAGAATGGGATAGAGAG
CTGGCTTCAAAGAAAAATCCTAAACTCATTAATGCCCTTCGGCGATGTTTTTTCTGGAGA
TTTATGTTCTATGGAATCTTTTTATATTTAGGGGAAGTCACCAAAGCAGTACAGCCTCTC
TTACTGGGAAGAATCATAGCTTCCTATGACCCGGATAACAAGGAGGAACGCTCTATCGCG
ATTTATCTAGGCATAGGCTTATGCCTTCTCTTTATTGTGAGGACACTGCTCCTACACCCA
GCCATTTTTGGCCTTCATCACATTGGAATGCAGATGAGAATAGCTATGTTTAGTTTGATT
TATAAGAAGACTTTAAAGCTGTCAAGCCGTGTTCTAGATAAAATAAGTATTGGACAACTT
GTTAGTCTCCTTTCCAACAACCTGAACAAATTTGATGAAGGACTTGCATTGGCACATTTC
GTGTGGATCGCTCCTTTGCAAGTGGCACTCCTCATGGGGCTAATCTGGGAGTTGTTACAG
GCGTCTGCCTTCTGTGGACTTGGTTTCCTGATAGTCCTTGCCCTTTTTCAGGCTGGGCTA
GGGAGAATGATGATGAAGTACAGAGATCAGAGAGCTGGGAAGATCAGTGAAAGACTCGTA
ATTACCTCAGAAATGATTGAGAACATCCAATCTGTTAAGGCATACTGCTGGGAAGAAGCA
ATGGAAAAAATGATTGAAAACTTAAGACAAACAGAACTGAAACTGACTCGGAAGGCAGCC
TATGTGAGATACTTCAATAGCTCAGCCTTCTTCTTCTCAGGGTTCTTTGTGGTGTTTTTA
TCTGTGCTTCCCTATGCACTAATCAAAGGAATCATCCTCCGGAAAATATTCACCACCATC
TCATTCTGCATTGTTCTGCGCATGGCGGTCACTCGGCAATTTCCCTGGGCTGTACAAACA
TGGTATGACTCTCTTGGAGCAATAAACAAAATACAGGATTTCTTACAAAAGCAAGAATAT
AAGACATTGGAATATAACTTAACGACTACAGAAGTAGTGATGGAGAATGTAACAGCCTTC
TGGGAGGAGGGATTTGGGGAATTATTTGAGAAAGCAAAACAAAACAATAACAATAGAAAA
ACTTCTAATGGTGATGACAGCCTCTTCTTCAGTAATTTCTCACTTCTTGGTACTCCTGTC
CTGAAAGATATTAATTTCAAGATAGAAAGAGGACAGTTGTTGGCGGTTGCTGGATCCACT
GGAGCAGGCAAGACTTCACTTCTAATGGTGATTATGGGAGAACTGGAGCCTTCAGAGGGT
AAAATTAAGCACAGTGGAAGAATTTCATTCTGTTCTCAGTTTTCCTGGATTATGCCTGGC
ACCATTAAAGAAAATATCATCTTTGGTGTTTCCTATGATGAATATAGATACAGAAGCGTC
ATCAAAGCATGCCAACTAGAAGAGGACATCTCCAAGTTTGCAGAGAAAGACAATATAGTT
CTTGGAGAAGGTGGAATCACACTGAGTGGAGGTCAACGAGCAAGAATTTCTTTAGCAAGA
GCAGTATACAAAGATGCTGATTTGTATTTATTAGACTCTCCTTTTGGATACCTAGATGTT
TTAACAGAAAAAGAAATATTTGAAAGCTGTGTCTGTAAACTGATGGCTAACAAAACTAGG
ATTTTGGTCACTTCTAAAATGGAACATTTAAAGAAAGCTGACAAAATATTAATTTTGCAT
GAAGGTAGCAGCTATTTTTATGGGACATTTTCAGAACTCCAAAATCTACAGCCAGACTTT
AGCTCAAAACTCATGGGATGTGATTCTTTCGACCAATTTAGTGCAGAAAGAAGAAATTCA
ATCCTAACTGAGACATTACACCGTTTCTCATTAGAAGGAGATGCTCCTGTCTCCTGGACA
GAAACAAAAAAACAATCTTTTAAACAGACTGGAGAGTTTGGGGAAAAAAGGAAGAATTCT
ATTCTCAATCCAATCAACTCTATACGAAAATTTTCCATTGTGCAAAAGACTCCCTTACAA
ATGAATGGCATCGAAGAGGATTCTGATGAGCCTTTAGAGAGAAGGCTGTCCTTAGTACCA
GATTCTGAGCAGGGAGAGGCGATACTGCCTCGCATCAGCGTGATCAGCACTGGCCCCACG
CTTCAGGCACGAAGGAGGCAGTCTGTCCTGAACCTGATGACACACTCAGTTAACCAAGGT
CAGAACATTCACCGAAAGACAACAGCATCCACACGAAAAGTGTCACTGGCCCCTCAGGCA
AACTTGACTGAACTGGATATATATTCAAGAAGGTTATCTCAAGAAACTGGCTTGGAAATA
AGTGAAGAAATTAACGAAGAAGACTTAAAGGAGTGCTTTTTTGATGATATGGAGAGCATA
CCAGCAGTGACTACATGGAACACATACCTTCGATATATTACTGTCCACAAGAGCTTAATT
TTTGTGCTAATTTGGTGCTTAGTAATTTTTCTGGCAGAGGTGGCTGCTTCTTTGGTTGTG
CTGTGGCTCCTTGGAAACACTCCTCTTCAAGACAAAGGGAATAGTACTCATAGTAGAAAT
AACAGCTATGCAGTGATTATCACCAGCACCAGTTCGTATTATGTGTTTTACATTTACGTG
GGAGTAGCCGACACTTTGCTTGCTATGGGATTCTTCAGAGGTCTACCACTGGTGCATACT
CTAATCACAGTGTCGAAAATTTTACACCACAAAATGTTACATTCTGTTCTTCAAGCACCT
ATGTCAACCCTCAACACGTTGAAAGCAGGTGGGATTCTTAATAGATTCTCCAAAGATATA
GCAATTTTGGATGACCTTCTGCCTCTTACCATATTTGACTTCATCCAGTTGTTATTAATT
GTGATTGGAGCTATAGCAGTTGTCGCAGTTTTACAACCCTACATCTTTGTTGCAACAGTG
CCAGTGATAGTGGCTTTTATTATGTTGAGAGCATATTTCCTCCAAACCTCACAGCAACTC
AAACAACTGGAATCTGAAGGCAGGAGTCCAATTTTCACTCATCTTGTTACAAGCTTAAAA
GGACTATGGACACTTCGTGCCTTCGGACGGCAGCCTTACTTTGAAACTCTGTTCCACAAA
GCTCTGAATTTACATACTGCCAACTGGTTCTTGTACCTGTCAACACTGCGCTGGTTCCAA
ATGAGAATAGAAATGATTTTTGTCATCTTCTTCATTGCTGTTACCTTCATTTCCATTTTA
ACAACAGGAGAAGGAGAAGGAAGAGTTGGTATTATCCTGACTTTAGCCATGAATATCATG
AGTACATTGCAGTGGGCTGTAAACTCCAGCATAGATGTGGATAGCTTGATGCGATCTGTG
AGCCGAGTCTTTAAGTTCATTGACATGCCAACAGAAGGTAAACCTACCAAGTCAACCAAA
CCATACAAGAATGGCCAACTCTCGAAAGTTATGATTATTGAGAATTCACACGTGAAGAAA
GATGACATCTGGCCCTCAGGGGGCCAAATGACTGTCAAAGATCTCACAGCAAAATACACA
GAAGGTGGAAATGCCATATTAGAGAACATTTCCTTCTCAATAAGTCCTGGCCAGAGGGTG
GGCCTCTTGGGAAGAACTGGATCAGGGAAGAGTACTTTGTTATCAGCTTTTTTGAGACTA
CTGAACACTGAAGGAGAAATCCAGATCGATGGTGTGTCTTGGGATTCAATAACTTTGCAA
CAGTGGAGGAAAGCCTTTGGAGTGATACCACAGAAAGTATTTATTTTTTCTGGAACATTT
AGAAAAAACTTGGATCCCTATGAACAGTGGAGTGATCAAGAAATATGGAAAGTTGCAGAT
GAGGTTGGGCTCAGATCTGTGATAGAACAGTTTCCTGGGAAGCTTGACTTTGTCCTTGTG
GATGGGGGCTGTGTCCTAAGCCATGGCCACAAGCAGTTGATGTGCTTGGCTAGATCTGTT
CTCAGTAAGGCGAAGATCTTGCTGCTTGATGAACCCAGTGCTCATTTGGATCCAGTAACA
TACCAAATAATTAGAAGAACTCTAAAACAAGCATTTGCTGATTGCACAGTAATTCTCTGT
GAACACAGGATAGAAGCAATGCTGGAATGCCAACAATTTTTGGTCATAGAAGAGAACAAA
GTGCGGCAGTACGATTCCATCCAGAAACTGCTGAACGAGAGGAGCCTCTTCCGGCAAGCC
ATCAGCCCCTCCGACAGGGTGAAGCTCTTTCCCCACCGGAACTCAAGCAAGTGCAAGTCT
AAGCCCCAGATTGCTGCTCTGAAAGAGGAGACAGAAGAAGAGGTGCAAGATACAAGGCTT
TAG
(pARM766)
SEQ ID NO: 2
ATGCAGAGGTCGCCTCTGGAGAAGGCCAGCGTTGTCTCCAAGCTGTTCTTCAGCTGGACC
AGACCAATTTTGAGGAAAGGATACAGACAGCGCCTGGAATTGTCAGACATATACCAAATC
CCTTCTGTTGATTCTGCTGACAATCTATCTGAGAAGTTGGAAAGAGAATGGGATAGAGAG
CTGGCTTCCAAGAAGAACCCTAAGCTCATTAATGCCCTTCGGCGATGCTTTTTCTGGAGG
TTCATGTTCTATGGAATCTTCCTGTACTTAGGGGAGGTCACCAAGGCAGTACAGCCTCTC
TTGCTGGGCAGAATCATAGCTTCCTATGACCCTGATAACAAGGAGGAACGCAGCATCGCG
ATCTACCTGGGCATCGGCTTGTGCCTGCTCTTTATCGTGAGGACACTGCTCCTACACCCT
GCCATCTTTGGCCTTCATCACATTGGAATGCAGATGAGAATCGCTATGTTCAGTTTGATT
TACAAGAAGACTTTAAAGCTGTCCAGCAGGGTGCTAGATAAGATCAGCATTGGACAGCTT
GTTAGCCTGCTTTCCAACAACCTGAACAAGTTCGATGAAGGACTGGCATTGGCACATTTC
GTGTGGATCGCTCCTCTGCAAGTGGCACTCCTGATGGGGTTGATCTGGGAGTTGCTGCAG
GCGAGCGCCTTCTGTGGACTTGGCTTCCTGATAGTCCTTGCCCTGTTCCAGGCTGGGCTA
GGGAGAATGATGATGAAGTACAGAGATCAGAGGGCTGGGAAGATCAGCGAGAGACTCGTG
ATCACCTCTGAGATGATCGAGAACATCCAGTCTGTGAAGGCATACTGCTGGGAAGAGGCA
ATGGAGAAGATGATTGAGAACTTAAGACAGACAGAGCTGAAGCTGACTCGGAAGGCAGCC
TATGTGAGATACTTCAACAGCTCAGCCTTCTTCTTCAGCGGGTTCTTTGTGGTCTTCCTG
TCTGTGCTTCCCTATGCACTAATCAAGGGAATCATTCTGCGGAAGATCTTCACAACCATC
TCCTTCTGCATTGTGCTGCGCATGGCGGTCACTCGGCAGTTTCCCTGGGCTGTACAGACA
TGGTATGACTCTCTGGGAGCCATCAACAAGATACAGGATTTCCTGCAGAAGCAAGAGTAT
AAGACATTGGAGTACAACTTAACGACTACAGAAGTAGTGATGGAGAACGTAACCGCCTTC
TGGGAGGAGGGATTTGGGGAGTTGTTCGAGAAAGCAAAGCAGAACAACAATAATCGGAAG
ACCTCCAATGGTGATGACAGCCTCTTCTTCAGTAACTTCAGCCTTCTTGGTACTCCTGTC
CTGAAGGACATCAACTTCAAGATAGAGAGGGGACAGTTGTTGGCGGTTGCTGGATCCACT
GGAGCAGGCAAGACTTCACTTCTAATGGTGATCATGGGAGAACTGGAGCCTAGCGAGGGC
AAGATCAAGCACAGTGGAAGGATCTCATTCTGTTCTCAGTTTTCCTGGATTATGCCTGGC
ACCATTAAGGAGAACATCATCTTTGGTGTTTCCTATGATGAGTACCGCTACAGAAGCGTC
ATCAAGGCATGCCAACTAGAAGAGGACATCTCCAAGTTTGCAGAGAAGGACAATATAGTT
CTTGGAGAAGGTGGAATCACACTGAGTGGAGGTCAACGAGCAAGAATCTCTTTAGCAAGA
GCAGTATACAAGGACGCTGATTTGTACTTGTTAGACTCTCCCTTTGGATACCTAGATGTG
CTGACCGAGAAGGAGATATTCGAAAGCTGTGTCTGTAAGCTGATGGCTAACAAGACTAGG
ATCTTGGTCACTTCTAAGATGGAACACCTGAAGAAAGCTGACAAGATCTTGATCCTGCAT
GAAGGTTCTAGCTACTTCTACGGGACATTTTCAGAACTCCAGAATCTACAGCCAGACTTT
AGCTCAAAGCTCATGGGATGTGATTCTTTCGACCAGTTTAGTGCAGAGAGACGGAACTCA
ATCCTAACTGAGACATTACACCGTTTCTCATTAGAAGGAGATGCTCCTGTCTCCTGGACA
GAGACGAAGAAACAGTCTTTTAAACAGACTGGAGAGTTTGGGGAGAAACGCAAGAACAGC
ATTCTCAATCCAATCAACTCTATACGAAAGTTCTCCATTGTGCAGAAGACTCCCTTACAG
ATGAATGGCATCGAAGAGGATTCTGATGAGCCTTTAGAGAGAAGGCTGTCCTTAGTACCA
GATTCTGAGCAGGGAGAGGCGATACTGCCTCGCATCAGCGTGATCAGCACTGGCCCCACG
CTTCAGGCACGAAGGCGCCAGTCTGTCCTGAACCTGATGACACACTCAGTTAACCAAGGT
CAGAACATTCACCGAAAGACAACCGCATCCACAAGGAAGGTGTCACTGGCCCCTCAGGCA
AACTTGACTGAACTGGACATCTACTCCAGAAGGTTATCTCAGGAGACTGGCTTGGAGATC
AGTGAAGAGATTAACGAAGAGGACTTAAAGGAGTGCTTCTTTGATGATATGGAGAGCATA
CCAGCAGTGACTACATGGAACACATACCTTAGGTACATCACTGTCCACAAGAGCCTGATC
TTCGTGCTAATTTGGTGCTTGGTGATCTTCCTGGCAGAGGTGGCTGCTTCTTTGGTTGTG
CTGTGGCTCCTTGGAAACACTCCTCTTCAAGACAAAGGGAATAGTACTCATTCCAGCAAC
AATTCCTATGCAGTGATTATCACCAGCACCAGTTCGTATTATGTGTTCTACATTTACGTG
GGAGTAGCCGACACTTTGCTTGCTATGGGATTCTTCAGAGGTCTACCACTGGTGCATACT
CTAATCACAGTGTCGAAGATCCTGCATCACAAGATGTTACATTCTGTTCTTCAAGCACCT
ATGTCAACCCTCAACACGTTGAAGGCAGGTGGGATTCTGAACAGGTTCTCCAAGGATATA
GCCATCCTGGATGACCTTCTGCCTCTTACCATCTTTGACTTCATCCAGTTGTTACTGATC
GTGATTGGAGCTATAGCAGTTGTCGCAGTGTTACAACCCTACATCTTCGTTGCAACAGTG
CCAGTGATAGTGGCTTTCATTATGTTGAGAGCATATTTCCTCCAAACCTCACAGCAACTC
AAGCAGCTGGAATCTGAAGGCAGGAGTCCAATTTTCACTCATCTTGTTACAAGCCTGAAG
GGACTCTGGACATTGCGTGCCTTCGGACGGCAGCCTTACTTTGAAACTCTGTTCCACAAA
GCTCTGAATTTACATACTGCCAACTGGTTCTTGTACCTGTCAACACTGCGCTGGTTCCAA
ATGAGAATAGAAATGATTTTTGTCATCTTCTTCATTGCTGTTACCTTCATTTCCATTTTA
ACAACAGGAGAAGGAGAAGGAAGAGTTGGTATTATCCTGACTTTAGCCATGAACATCATG
AGTACATTGCAGTGGGCTGTGAACTCCAGCATAGATGTGGATAGCTTGATGCGATCTGTG
AGCCGAGTCTTCAAGTTCATTGACATGCCCACCGAGGGTAAGCCTACCAAGTCCACCAAG
CCCTACAAGAATGGCCAACTCTCGAAGGTTATGATCATTGAGAATTCACACGTGAAGAAA
GATGACATCTGGCCCTCAGGGGGCCAAATGACTGTCAAAGATCTCACAGCCAAGTACACA
GAAGGTGGAAATGCCATCCTGGAGAACATTTCCTTCAGCATCAGTCCTGGCCAGAGGGTG
GGCCTCTTGGGAAGAACTGGATCAGGGAAGAGTACTTTGTTATCAGCCTTCTTGAGACTA
CTGAACACTGAAGGCGAGATCCAGATCGATGGTGTGTCTTGGGACAGCATCACTTTGCAA
CAGTGGAGGAAGGCCTTCGGCGTGATACCACAGAAGGTGTTCATCTTCTCCGGAACCTTC
AGGAAGAACTTGGATCCCTATGAACAGTGGAGTGATCAGGAGATCTGGAAGGTTGCAGAT
GAGGTTGGGCTCAGATCTGTGATAGAACAGTTTCCTGGGAAGCTTGACTTTGTCCTTGTG
GATGGGGGCTGTGTCCTAAGCCACGGCCACAAGCAGTTGATGTGCTTGGCTAGATCTGTT
CTCAGTAAGGCGAAGATCTTGCTGCTTGATGAACCCAGTGCTCATTTGGATCCAGTAACA
TACCAGATCATTCGGAGAACTCTGAAGCAGGCATTTGCTGATTGCACAGTAATTCTCTGT
GAACACAGGATAGAAGCAATGCTGGAATGCCAACAGTTCTTGGTCATCGAAGAGAACAAG
GTGCGGCAGTACGATTCCATCCAGAAGCTGCTGAACGAGAGGAGCCTCTTCCGGCAAGCC
ATCAGCCCCTCCGACAGGGTGAAGCTCTTTCCCCACCGGAACTCAAGCAAGTGCAAGTCT
AAGCCCCAGATCGCCGCTCTGAAGGAAGAGACTGAGGAAGAGGTGCAGGATACCAGGCTG
TGA
(pARM1831)
SEQ ID NO: 3
ATGCAGCGCAGCCCCCTCGAGAAGGCCAGCGTGGTGAGCAAGCTGTTCTTCAGCTGGACC
CGCCCCATCCTGCGCAAGGGCTACCGCCAGCGCCTGGAGCTGAGCGACATCTACCAGATC
CCCAGCGTGGACAGCGCCGACAACCTGAGCGAGAAGCTGGAGCGCGAGTGGGACCGCGAG
CTGGCCAGCAAGAAGAACCCCAAGCTGATCAACGCCCTGCGCCGCTGCTTCTTCTGGCGC
TTCATGTTCTACGGCATCTTCCTGTACCTGGGCGAGGTGACCAAGGCCGTGCAGCCCCTG
CTGCTGGGCCGCATCATCGCCAGCTACGACCCCGACAACAAGGAGGAGCGCAGCATCGCC
ATCTACCTGGGCATCGGCCTGTGCCTGCTGTTCATCGTGCGCACCCTGCTGCTGCACCCC
GCCATCTTCGGCCTGCACCACATCGGCATGCAGATGCGCATCGCCATGTTCAGCCTGATC
TACAAGAAGACCCTGAAGCTGAGCAGCCGCGTGCTGGACAAGATCAGCATCGGCCAGCTG
GTGAGCCTGCTGAGCAACAACCTGAACAAGTTCGACGAGGGCCTGGCCCTGGCCCACTTC
GTGTGGATCGCCCCCCTGCAGGTGGCCCTGCTGATGGGCCTGATCTGGGAGCTGCTGCAG
GCCAGCGCCTTCTGCGGCCTGGGCTTCCTGATCGTGCTGGCCCTGTTCCAGGCCGGCCTG
GGCCGCATGATGATGAAGTACCGCGACCAGCGCGCCGGCAAGATCAGCGAGCGCCTGGTG
ATCACCAGCGAGATGATCGAGAACATCCAGAGCGTGAAGGCCTACTGCTGGGAGGAGGCC
ATGGAGAAGATGATCGAGAACCTGCGCCAGACCGAGCTGAAGCTGACCCGCAAGGCCGCC
TACGTGCGCTACTTCAACAGCAGCGCCTTCTTCTTCAGCGGCTTCTTCGTGGTGTTCCTG
AGCGTGCTGCCCTACGCCCTGATCAAGGGCATCATCCTGCGCAAGATCTTCACCACCATC
AGCTTCTGCATCGTGCTGCGCATGGCCGTGACCCGCCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGCTTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCCGTG
CTGAAGGACATCAACTTCAAGATCGAGCGCGGCCAGCTGCTGGCCGTGGCCGGCAGCACC
GGCGCCGGCAAGACCAGCCTGCTGATGGTGATCATGGGCGAGCTGGAGCCCAGCGAGGGC
AAGATCAAGCACAGCGGCCGCATCAGCTTCTGCAGCCAGTTCAGCTGGATCATGCCCGGC
ACCATCAAGGAGAACATCATCTTCGGCGTGAGCTACGACGAGTACCGCTACCGCAGCGTG
ATCAAGGCCTGCCAGCTGGAGGAGGACATCAGCAAGTTCGCCGAGAAGGACAACATCGTG
CTGGGCGAGGGCGGCATCACCCTGAGCGGCGGCCAGCGCGCCCGCATCAGCCTGGCCCGC
GCCGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCCGC
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGCGCCGCAACAGC
ATCCTGACCGAGACCCTGCACCGCTTCAGCCTGGAGGGCGACGCCCCCGTGAGCTGGACC
GAGACCAAGAAGCAGAGCTTCAAGCAGACCGGCGAGTTCGGCGAGAAGCGCAAGAACAGC
ATCCTGAACCCCATCAACAGCATCCGCAAGTTCAGCATCGTGCAGAAGACCCCCCTGCAG
ATGAACGGCATCGAGGAGGACAGCGACGAGCCCCTGGAGCGCCGCCTGAGCCTGGTGCCC
GACAGCGAGCAGGGCGAGGCCATCCTGCCCCGCATCAGCGTGATCAGCACCGGCCCCACC
CTGCAGGCCCGCCGCCGCCAGAGCGTGCTGAACCTGATGACCCACAGCGTGAACCAGGGC
CAGAACATCCACCGCAAGACCACCGCCAGCACCCGCAAAGTGAGCCTGGCCCCCCAGGCC
AACCTGACCGAGCTGGACATCTACAGCCGCCGCCTGAGCCAGGAGACCGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCCCTGCAGGACAAGGGCAACAGCACCCACAGCCGCAAC
AACAGCTACGCCGTGATCATCACCAGCACCAGCAGCTACTACGTGTTCTACATCTACGTG
GGCGTGGCCGACACCCTGCTGGCCATGGGCTTCTTCCGCGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGCATCCTGAACCGCTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCCGTGATCGTGGCCTTCATCATGCTGCGCGCCTACTTCCTGCAGACCAGCCAGCAGCTG
AAGCAGCTGGAGAGCGAGGGCCGCAGCCCCATCTTCACCCACCTGGTGACCAGCCTGAAG
GGCCTGTGGACCCTGCGCGCCTTCGGCCGCCAGCCCTACTTCGAGACCCTGTTCCACAAG
GCCCTGAACCTGCACACCGCCAACTGGTTCCTGTACCTGAGCACCCTGCGCTGGTTCCAG
ATGCGCATCGAGATGATCTTCGTGATCTTCTTCATCGCCGTGACCTTCATCAGCATCCTG
ACCACCGGCGAGGGCGAGGGCCGCGTGGGCATCATCCTGACCCTGGCCATGAACATCATG
AGCACCCTGCAGTGGGCCGTGAACAGCAGCATCGACGTGGACAGCCTGATGCGCAGCGTG
AGCCGCGTGTTCAAGTTCATCGACATGCCCACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGCGCGTG
GGCCTGCTGGGCCGCACCGGCAGCGGCAAGAGCACCCTGCTGAGCGCCTTCCTGCGCCTG
CTGAACACCGAGGGCGAGATCCAGATCGACGGCGTGAGCTGGGACAGCATCACCCTGCAG
CAGTGGCGCAAGGCCTTCGGCGTGATCCCCCAGAAGGTGTTCATCTTCAGCGGCACCTTC
CGCAAGAACCTGGACCCCTACGAGCAGTGGAGCGACCAGGAGATCTGGAAGGTGGCCGAC
GAGGTGGGCCTGCGCAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCCGCAGCGTG
CTGAGCAAGGCCAAGATCCTGCTGCTGGACGAGCCCAGCGCCCACCTGGACCCCGTGACC
TACCAGATCATCCGCCGCACCCTGAAGCAGGCCTTCGCCGACTGCACCGTGATCCTGTGC
GAGCACCGCATCGAGGCCATGCTGGAGTGCCAGCAGTTCCTGGTGATCGAGGAGAACAAG
GTGCGCCAGTACGACAGCATCCAGAAGCTGCTGAACGAGCGCAGCCTGTTCCGCCAGGCC
ATCAGCCCCAGCGACCGCGTGAAGCTTTTCCCCCACCGCAACAGCAGCAAGTGCAAGAGC
AAGCCCCAGATCGCCGCCCTGAAGGAGGAGACCGAGGAGGAGGTGCAGGACACCCGCCTG
TAG
(pARM1832)
SEQ ID NO: 4
ATGCAGAGGTCGCCTCTGGAGAAGGCCAGCGTTGTCTCCAAGCTGTTCTTCAGCTGGACC
AGACCAATTTTGAGGAAAGGATACAGACAGCGCCTGGAATTGTCAGACATATACCAAATC
CCTTCTGTTGATTCTGCTGACAATCTATCTGAGAAGTTGGAAAGAGAATGGGATAGAGAG
CTGGCTTCCAAGAAGAACCCTAAGCTCATTAATGCCCTTCGGCGATGCTTTTTCTGGAGG
TTCATGTTCTATGGAATCTTCCTGTACTTAGGGGAGGTCACCAAGGCAGTACAGCCTCTC
TTGCTGGGCAGAATCATAGCTTCCTATGACCCGGATAACAAGGAGGAACGCTCTATCGCG
ATTTATCTAGGCATAGGCTTATGCCTTCTCTTTATTGTGAGGACACTGCTCCTACACCCA
GCCATTTTTGGCCTTCATCACATTGGAATGCAGATGAGAATAGCTATGTTTAGTTTGATT
TATAAGAAGACTTTAAAGCTGTCAAGCCGTGTTCTAGATAAAATAAGTATTGGACAACTT
GTTAGTCTCCTTTCCAACAACCTGAACAAATTTGATGAAGGACTTGCATTGGCACATTTC
GTGTGGATCGCTCCTCTGCAAGTGGCACTCCTGATGGGGTTGATCTGGGAGTTGCTGCAG
GCGAGCGCCTTCTGTGGACTTGGCTTCCTGATAGTCCTTGCCCTGTTCCAGGCTGGGCTA
GGGAGAATGATGATGAAGTACAGAGATCAGAGGGCTGGGAAGATCAGCGAGAGACTCGTG
CACCTCTGAGATGATCGATAGAACATCCAGTCTGTGAAGGCATACTGCTGGGAAGAGGCA
ATGGAGAAGATGATTGAGAACTTAAGACAGACAGAGCTGAAGCTGACTCGGAAGGCAGCC
TATGTGAGATACTTCAACAGCTCAGCCTTCTTCTTCAGCGGGTTCTTTGTGGTCTTCCTG
TCTGTGCTTCCCTATGCACTAATCAAGGGAATCATTCTGCGGAAGATCTTCACAACCATC
TCCTTCTGCATTGTGCTGCGCATGGCGGTCACTCGGCAGTTTCCCTGGGCTGTACAGACA
TGGTATGACTCTCTGGGAGCCATCAACAAGATACAGGATTTCCTGCAGAAGCAAGAGTAT
AAGACATTGGAGTACAACTTAACGACTACAGAAGTAGTGATGGAGAACGTAACCGCCTTC
TGGGAGGAGGGATTTGGGGAGTTGTTCGAGAAAGCAAAGCAGAACAACAATAATCGGAAG
ACCTCCAATGGTGATGACAGCCTCTTCTTCAGTAACTTCAGCCTTCTTGGTACTCCTGTC
CTGAAGGACATCAACTTCAAGATAGAGAGGGGACAGTTGTTGGCGGTTGCTGGATCCACT
GGAGCAGGCAAGACTTCACTTCTAATGGTGATCATGGGAGAACTGGAGCCTAGCGAGGGC
AAGATCAAGCACAGTGGAAGGATCTCATTCTGTTCTCAGTTTTCCTGGATTATGCCTGGC
ACCATTAAGGAGAACATCATCTTTGGTGTTTCCTATGATGAGTACCGCTACAGAAGCGTC
ATCAAGGCATGCCAACTAGAAGAGGACATCTCCAAGTTTGCAGAGAAGGACAATATAGTT
CTTGGAGAAGGTGGAATCACACTGAGTGGAGGTCAACGAGCAAGAATCTCTTTAGCAAGA
GCAGTATACAAGGACGCTGATTTGTACTTGTTAGACTCTCCCTTTGGATACCTAGATGTG
CTGACCGAGAAGGAGATATTCGAAAGCTGTGTCTGTAAGCTGATGGCTAACAAGACTAGG
ATCTTGGTCACTTCTAAGATGGAACACCTGAAGAAAGCTGACAAGATCTTGATCCTGCAT
GAAGGTTCTAGCTACTTCTACGGGACATTTTCAGAACTCCAGAATCTACAGCCAGACTTT
AGCTCAAAGCTCATGGGATGTGATTCTTTCGACCAGTTTAGTGCAGAGAGACGGAACTCA
ATCCTAACTGAGACATTACACCGTTTCTCATTAGAAGGAGATGCTCCTGTCTCCTGGACA
GAGACGAAGAAACAGTCTTTTAAACAGACTGGAGAGTTTGGGGAGAAACGCAAGAACAGC
ATTCTCAATCCAATCAACTCTATACGAAAGTTCTCCATTGTGCAGAAGACTCCCTTACAG
ATGAATGGCATCGAAGAGGATTCTGATGAGCCTTTAGAGAGAAGGCTGTCCTTAGTACCA
GATTCTGAGCAGGGAGAGGCGATACTGCCTCGCATCAGCGTGATCAGCACTGGCCCCACG
CTTCAGGCACGAAGGCGCCAGTCTGTCCTGAACCTGATGACACACTCAGTTAACCAAGGT
CAGAACATTCACCGAAAGACAACCGCATCCACAAGGAAGGTGTCACTGGCCCCTCAGGCA
AACTTGACTGAACTGGACATCTACTCCAGAAGGTTATCTCAGGAGACTGGCTTGGAGATC
AGTGAAGAGATTAACGAAGAGGACTTAAAGGAGTGCTTCTTTGATGATATGGAGAGCATA
CCAGCAGTGACTACATGGAACACATACCTTAGGTACATCACTGTCCACAAGAGCCTGATC
TTCGTGCTAATTTGGTGCTTGGTGATCTTCCTGGCAGAGGTGGCTGCTTCTTTGGTTGTG
CTGTGGCTCCTTGGAAACACTCCTCTTCAAGACAAAGGGAATAGTACTCATTCCAGCAAC
AATTCCTATGCAGTGATTATCACCAGCACCAGTTCGTATTATGTGTTCTACATTTACGTG
GGAGTAGCCGACACTTTGCTTGCTATGGGATTCTTCAGAGGTCTACCACTGGTGCATACT
CTAATCACAGTGTCGAAGATCCTGCATCACAAGATGTTACATTCTGTTCTTCAAGCACCT
ATGTCAACCCTCAACACGTTGAAGGCAGGTGGGATTCTGAACAGGTTCTCCAAGGATATA
GCCATCCTGGATGACCTTCTGCCTCTTACCATCTTTGACTTCATCCAGTTGTTACTGATC
GTGATTGGAGCTATAGCAGTTGTCGCAGTGTTACAACCCTACATCTTCGTTGCAACAGTG
CCAGTGATAGTGGCTTTCATTATGTTGAGAGCATATTTCCTCCAAACCTCACAGCAACTC
AAGCAGCTGGAATCTGAAGGCAGGAGTCCAATTTTCACTCATCTTGTTACAAGCCTGAAG
GGACTCTGGACATTGCGTGCCTTCGGACGGCAGCCTTACTTTGAAACTCTGTTCCACAAA
GCTCTGAATTTACATACTGCCAACTGGTTCTTGTACCTGTCAACACTGCGCTGGTTCCAA
ATGAGAATAGAAATGATTTTTGTCATCTTCTTCATTGCTGTTACCTTCATTTCCATTTTA
ACAACAGGAGAAGGAGAAGGAAGAGTTGGTATTATCCTGACTTTAGCCATGAACATCATG
AGTACATTGCAGTGGGCTGTGAACTCCAGCATAGATGTGGATAGCTTGATGCGATCTGTG
AGCCGAGTCTTCAAGTTCATTGACATGCCCACCGAGGGTAAGCCTACCAAGTCCACCAAG
CCCTACAAGAATGGCCAACTCTCGAAGGTTATGATCATTGAGAATTCACACGTGAAGAAA
GATGACATCTGGCCCTCAGGGGGCCAAATGACTGTCAAAGATCTCACAGCCAAGTACACA
GAAGGTGGAAATGCCATCCTGGAGAACATTTCCTTCAGCATCAGTCCTGGCCAGAGGGTG
GGCCTCTTGGGAAGAACTGGATCAGGGAAGAGTACTTTGTTATCAGCCTTCTTGAGACTA
CTGAACACTGAAGGCGAGATCCAGATCGATGGTGTGTCTTGGGACAGCATCACTTTGCAA
CAGTGGAGGAAGGCCTTCGGCGTGATACCACAGAAGGTGTTCATCTTCTCCGGAACCTTC
AGGAAGAACTTGGATCCCTATGAACAGTGGAGTGATCAGGAGATCTGGAAGGTTGCAGAT
GAGGTTGGGCTCAGATCTGTGATAGAACAGTTTCCTGGGAAGCTTGACTTTGTCCTTGTG
GATGGGGGCTGTGTCCTAAGCCACGGCCACAAGCAGTTGATGTGCTTGGCTAGATCTGTT
CTCAGTAAGGCGAAGATCTTGCTGCTTGATGAACCCAGTGCTCATTTGGATCCAGTAACA
TACCAGATCATTCGGAGAACTCTGAAGCAGGCATTTGCTGATTGCACAGTAATTCTCTGT
GAACACAGGATAGAAGCAATGCTGGAATGCCAACAGTTCTTGGTCATCGAAGAGAACAAG
GTGCGGCAGTACGATTCCATCCAGAAGCTGCTGAACGAGAGGAGCCTCTTCCGGCAAGCC
ATCAGCCCCTCCGACAGGGTGAAGCTCTTTCCCCACCGGAACTCAAGCAAGTGCAAGTCT
AAGCCCCAGATCGCCGCTCTGAAGGAAGAGACTGAGGAAGAGGTGCAGGATACCAGGCTG
TAG
(pARM1833)
SEQ ID NO: 5
ATGCAGAGGAGCCCCCTGGAGAAGGCCAGCGTGGTGAGCAAGCTGTTCTTCAGCTGGACC
AGGCCCATCCTGAGGAAGGGCTACAGGCAGAGGCTGGAGCTGAGCGACATCTACCAGATC
CCCAGCGTGGACAGCGCCGACAACCTGAGCGAGAAGCTGGAGAGGGAGTGGGACAGGGAG
CTGGCCAGCAAGAAGAACCCCAAGCTGATCAACGCCCTGCGGAGGTGCTTCTTCTGGAGG
TTCATGTTCTACGGCATCTTCCTGTACCTGGGCGAGGTGACCAAGGCCGTGCAGCCCCTG
CTGCTGGGCAGGATCATCGCCAGCTACGACCCCGACAACAAGGAGGAGAGGAGCATCGCC
ATCTACCTGGGCATCGGCCTGTGCCTGCTGTTCATCGTGAGGACCCTGCTGCTGCACCCC
GCCATCTTCGGCCTGCACCACATCGGCATGCAGATGAGGATCGCCATGTTCAGCCTGATC
TACAAGAAGACCCTGAAGCTGAGCAGCAGGGTGCTGGACAAGATCAGCATCGGCCAGCTG
GTGAGCCTGCTGAGCAACAACCTGAACAAGTTCGACGAGGGCCTGGCCCTGGCCCACTTC
GTGTGGATCGCCCCCCTGCAGGTGGCCCTGCTGATGGGCCTGATCTGGGAGCTGCTGCAG
GCCAGCGCCTTCTGCGGCCTGGGCTTCCTGATCGTGCTGGCCCTGTTCCAGGCCGGCCTG
GGCAGGATGATGATGAAGTACAGGGACCAGAGGGCCGGCAAGATCAGCGAGAGGCTGGTG
ATCACCAGCGAGATGATCGAGAACATCCAGAGCGTGAAGGCCTACTGCTGGGAGGAGGCC
ATGGAGAAGATGATCGAGAACCTGAGGCAGACCGAGCTGAAGCTGACCCGGAAGGCCGCC
TACGTGAGGTACTTCAACAGCAGCGCCTTCTTCTTCAGCGGCTTCTTCGTGGTGTTCCTG
AGCGTGCTGCCCTACGCCCTGATCAAGGGCATCATCCTGCGGAAGATCTTCACCACCATC
AGCTTCTGCATCGTGCTGAGGATGGCCGTGACCCGGCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGCTTCGGCGAGCTGTTCGAGAAGGCCAAGCAGAACAACAACAACAGGAAG
ACCAGCAACGGCGACGACAGCCTGTTCTTCAGCAACTTCAGCCTGCTGGGCACCCCCGTG
CTGAAGGACATCAACTTCAAGATCGAGAGGGGCCAGCTGCTGGCCGTGGCCGGCAGCACC
GGCGCCGGCAAGACCAGCCTGCTGATGGTGATCATGGGCGAGCTGGAGCCCAGCGAGGGC
AAGATCAAGCACAGCGGCAGGATCAGCTTCTGCAGCCAGTTCAGCTGGATCATGCCCGGC
ACCATCAAGGAGAACATCATCTTCGGCGTGAGCTACGACGAGTACAGGTACAGGAGCGTG
ATCAAGGCCTGCCAGCTGGAGGAGGACATCAGCAAGTTCGCCGAGAAGGACAACATCGTG
CTGGGCGAGGGCGGCATCACCCTGAGCGGCGGCCAGAGGGCCAGGATCAGCCTGGCCAGG
GCCGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGAGGAGGAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGCGAGTTCGGCGAGAAGAGGAAGAACAGC
ATCCTGAACCCCATCAACAGCATCAGGAAGTTCAGCATCGTGCAGAAGACCCCCCTGCAG
ATGAACGGCATCGAGGAGGACAGCGACGAGCCCCTGGAGAGGAGGCTGAGCCTGGTGCCC
GACAGCGAGCAGGGCGAGGCCATCCTGCCCAGGATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCCCAGGCC
AACCTGACCGAGCTGGACATCTACAGCAGGAGGCTGAGCCAGGAGACCGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCCCTGCAGGACAAGGGCAACAGCACCCACAGCAGGAAC
AACAGCTACGCCGTGATCATCACCAGCACCAGCAGCTACTACGTGTTCTACATCTACGTG
GGCGTGGCCGACACCCTGCTGGCCATGGGCTTCTTCAGGGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGCATCCTGAACAGGTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCCGTGATCGTGGCCTTCATCATGCTGAGGGCCTACTTCCTGCAGACCAGCCAGCAGCTG
AAGCAGCTGGAGAGCGAGGGCAGGAGCCCCATCTTCACCCACCTGGTGACCAGCCTGAAG
GGCCTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGAGGTGGTTCCAG
ATGAGGATCGAGATGATCTTCGTGATCTTCTTCATCGCCGTGACCTTCATCAGCATCCTG
ACCACCGGCGAGGGCGAGGGCAGGGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCCACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGCAGGACCGGCAGCGGCAAGAGCACCCTGCTGAGCGCCTTCCTGAGGCTG
CTGAACACCGAGGGCGAGATCCAGATCGACGGCGTGAGCTGGGACAGCATCACCCTGCAG
CAGTGGAGGAAGGCCTTCGGCGTGATCCCCCAGAAGGTGTTCATCTTCAGCGGCACCTTC
AGGAAGAACCTGGACCCCTACGAGCAGTGGAGCGACCAGGAGATCTGGAAGGTGGCCGAC
GAGGTGGGCCTGAGGAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCAGGAGCGTG
CTGAGCAAGGCCAAGATCCTGCTGCTGGACGAGCCCAGCGCCCACCTGGACCCCGTGACC
TACCAGATCATCAGGAGGACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM1834)
SEQ ID NO: 6
ATGCAGAGGTCGCCCCTGGAGAAGGCCAGCGTGGTGTCCAAGCTGTTCTTCAGCTGGACC
AGGCCCATCCTGAGGAAGGGCTACAGGCAGAGGCTGGAGCTGTCAGACATCTACCAGATC
CCCTCTGTGGACAGCGCTGACAACCTGTCTGAGAAGCTGGAGAGGGAGTGGGACAGGGAG
CTGGCCAGCAAGAAGAACCCCAAGCTGATCAACGCCCTGCGGAGGTGCTTCTTCTGGAGG
TTCATGTTCTACGGAATCTTCCTGTACCTGGGGGAGGTGACCAAGGCCGTGCAGCCCCTG
CTGCTGGGAAGGATCATCGCCTCCTACGACCCCGACAACAAGGAGGAGAGGAGCATCGCC
ATCTACCTGGGCATCGGCCTGTGCCTGCTGTTCATCGTGAGGACCCTGCTGCTGCACCCC
GCCATCTTCGGCCTGCACCACATCGGAATGCAGATGAGGATCGCCATGTTCAGCCTGATC
TACAAGAAGACCCTGAAGCTGTCAAGCAGGGTGCTGGACAAGATCAGTATCGGACAGCTG
GTGAGTCTGCTGTCCAACAACCTGAACAAGTTCGACGAGGGACTGGCCCTGGCCCACTTC
GTGTGGATCGCTCCCCTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGGATGATGATGAAGTACAGGGACCAGAGGGCTGGCAAGATCAGCGAGAGGCTGGTG
ATCACCTCAGAGATGATCGAGAACATCCAGAGCGTGAAGGCATACTGCTGGGAGGAGGCC
ATGGAGAAGATGATCGAGAACCTGAGGCAGACCGAGCTGAAGCTGACCCGGAAGGCCGCC
TACGTGAGGTACTTCAACAGCAGCGCCTTCTTCTTCTCAGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGAGGATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGGAAG
ACCAGCAACGGCGACGACAGCCTGTTCTTCAGCAACTTCAGCCTGCTGGGCACCCCCGTG
CTGAAGGACATCAACTTCAAGATCGAGAGGGGACAGCTGCTGGCCGTGGCCGGAAGCACC
GGAGCCGGCAAGACCTCACTGCTGATGGTGATCATGGGAGAGCTGGAGCCCTCAGAGGGC
AAGATCAAGCACAGTGGAAGGATCTCATTCTGCTCTCAGTTCAGCTGGATCATGCCCGGC
ACCATCAAGGAGAACATCATCTTCGGTGTGTCCTACGACGAGTACAGGTACAGGAGCGTG
ATCAAGGCCTGCCAGCTGGAGGAGGACATCAGCAAGTTCGCAGAGAAGGACAACATCGTG
CTGGGAGAGGGTGGCATCACCCTGAGCGGAGGCCAGAGGGCCAGGATCAGCCTGGCAAGG
GCAGTGTACAAGGACGCTGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCTCTAAGATGGAGCACCTGAAGAAGGCTGACAAGATCCTGATCCTGCAC
GAGGGCAGCAGCTACTTCTACGGGACCTTCAGCGAGCTGCAGAACCTGCAGCCCGACTTC
AGCAGCAAGCTGATGGGCTGCGACAGCTTCGACCAGTTCAGCGCCGAGAGGAGGAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCCATCAACAGCATCAGGAAGTTCAGCATCGTGCAGAAGACCCCCCTGCAG
ATGAACGGCATCGAGGAGGACAGCGACGAGCCCCTGGAGAGGAGGCTGTCCCTGGTGCCC
GACAGCGAGCAGGGCGAGGCCATCCTGCCCAGGATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCCCAGGCC
AACCTGACCGAGCTGGACATCTACAGCAGGAGGCTGAGCCAGGAGACCGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCCCTGCAGGACAAGGGCAACAGCACCCACAGCAGGAAC
AACAGCTACGCCGTGATCATCACCAGCACCAGCAGCTACTACGTGTTCTACATCTACGTG
GGAGTGGCCGACACCCTGCTGGCCATGGGCTTCTTCAGGGGTCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGTGGGATCCTGAACAGGTTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCCGTGATCGTGGCCTTCATCATGCTGAGGGCCTACTTCCTGCAGACCAGCCAGCAGCTG
AAGCAGCTGGAGTCTGAGGGCAGGAGTCCCATCTTCACCCACCTGGTGACCAGCCTGAAG
GGACTGTGGACCCTGAGGGCCTTCGGCCGGCAGCCCTACTTCGAGACCCTGTTCCACAAG
GCTCTGAACCTGCACACCGCCAACTGGTTCCTGTACCTGAGCACCCTGAGGTGGTTCCAG
ATGAGGATCGAGATGATCTTCGTGATCTTCTTCATCGCCGTGACCTTCATCTCCATCCTG
ACCACCGGCGAGGGAGAGGGAAGGGTGGGCATCATCCTGACCCTGGCCATGAACATCATG
AGCACCCTGCAGTGGGCTGTGAACAGCAGCATCGACGTGGACAGCCTGATGAGGAGCGTG
AGCAGGGTGTTCAAGTTCATCGACATGCCCACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCTCAATCAGCCCCGGCCAGAGGGTG
GGCCTGCTGGGAAGGACCGGCTCAGGCAAGAGCACCCTGCTGAGCGCCTTCCTGAGGCTG
CTGAACACCGAGGGCGAGATCCAGATCGACGGCGTGAGCTGGGACTCAATCACCCTGCAG
CAGTGGAGGAAGGCCTTCGGCGTGATCCCCCAGAAGGTGTTCATCTTCTCTGGAACCTTC
AGGAAGAACCTGGACCCCTACGAGCAGTGGAGCGACCAGGAGATCTGGAAGGTGGCCGAC
GAGGTGGGCCTGAGGAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGGAGCGTG
CTGAGCAAGGCCAAGATCCTGCTGCTGGACGAGCCCAGTGCCCACCTGGACCCCGTGACC
TACCAGATCATCAGGAGGACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACTCCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM1835)
SEQ ID NO: 7
ATGCAGAGGTCGCCTCTGGAGAAGGCCAGCGTGGTGTCCAAGCTGTTCTTCAGCTGGACC
AGACCAATCCTGAGGAAGGGCTACAGACAGCGCCTGGAGCTGTCAGACATCTACCAGATC
CCTTCTGTGGACTCTGCTGACAACCTGTCTGAGAAGCTGGAGAGAGAGTGGGACAGAGAG
CTGGCCAGCAAGAAGAACCCTAAGCTGATCAACGCCCTGCGGAGGTGCTTCTTCTGGAGA
TTCATGTTCTACGGAATCTTCCTGTACCTGGGGGAGGTGACCAAGGCCGTGCAGCCTCTG
CTGCTGGGAAGAATCATCGCCTCCTACGACCCCGACAACAAGGAGGAGCGCTCTATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGTATCGGACAGCTG
GTGAGTCTGCTGTCCAACAACCTGAACAAGTTCGACGAGGGACTGGCCCTGGCCCACTTC
GTGTGGATCGCTCCTCTGCAGGTGGCCCTGCTGATGGGGCTGATCTGGGAGCTGCTGCAG
GCCTCTGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCTCAGAGATGATCGAGAACATCCAGTCTGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCTCAGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCTCTAACGGCGACGACAGCCTGTTCTTCAGCAACTTCAGCCTGCTGGGCACCCCTGTG
CTGAAGGACATCAACTTCAAGATCGAGAGAGGACAGCTGCTGGCCGTGGCCGGATCCACC
GGAGCCGGCAAGACCTCACTGCTGATGGTGATCATGGGAGAGCTGGAGCCTTCAGAGGGC
AAGATCAAGCACAGTGGAAGAATCTCATTCTGCTCTCAGTTCTCCTGGATCATGCCTGGC
ACCATCAAGGAGAACATCATCTTCGGTGTGTCCTACGACGAGTACAGATACAGAAGCGTG
ATCAAGGCCTGCCAGCTGGAGGAGGACATCTCCAAGTTCGCAGAGAAGGACAACATCGTG
CTGGGAGAGGGTGGCATCACCCTGAGCGGAGGCCAGAGGGCCAGAATCTCTCTGGCAAGA
GCAGTGTACAAGGACGCTGACCTGTACCTGCTGGACTCTCCTTTCGGATACCTGGACGTG
CTGACCGAGAAGGAGATCTTCGAGAGCTGCGTGTGCAAGCTGATGGCCAACAAGACCAGG
ATCCTGGTGACCTCTAAGATGGAGCACCTGAAGAAGGCTGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACTCTTTCGACCAGTTCAGCGCCGAGAGAAGAAACAGC
ATCCTGACCGAGACCCTGCACAGGTTCAGCCTGGAGGGCGACGCCCCTGTGTCCTGGACC
GAGACCAAGAAGCAGTCTTTCAAGCAGACCGGAGAGTTCGGCGAGAAGAGGAAGAACTCT
ATCCTGAACCCAATCAACTCTATCAGGAAGTTCTCCATCGTGCAGAAGACCCCCCTGCAG
ATGAACGGCATCGAGGAGGACTCTGACGAGCCTCTGGAGAGAAGGCTGTCCCTGGTGCCA
GACTCTGAGCAGGGCGAGGCCATCCTGCCTCGCATCAGCGTGATCAGCACCGGCCCCACC
CTGCAGGCCAGGAGGAGGCAGTCTGTGCTGAACCTGATGACCCACAGCGTGAACCAGGGC
CAGAACATCCACAGGAAGACCACCGCCTCCACCAGGAAGGTGAGCCTGGCCCCTCAGGCC
AACCTGACCGAGCTGGACATCTACAGCAGAAGGCTGTCTCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCTCTCTGGTGGTG
CTGTGGCTGCTGGGCAACACCCCTCTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCACACC
CTGATCACCGTGAGCAAGATCCTGCACCACAAGATGCTGCACTCTGTGCTGCAGGCCCCT
ATGAGCACCCTGAACACCCTGAAGGCCGGTGGGATCCTGAACAGATTCTCCAAGGACATC
GCCATCCTGGACGACCTGCTGCCTCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGTCTGAGGGCAGGAGTCCAATCTTCACCCACCTGGTGACCAGCCTGAAG
GGACTGTGGACCCTGAGGGCCTTCGGCCGGCAGCCTTACTTCGAGACCCTGTTCCACAAG
GCTCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCTCCATCCTG
ACCACCGGCGAGGGAGAGGGAAGAGTGGGCATCATCCTGACCCTGGCCATGAACATCATG
AGCACCCTGCAGTGGGCTGTGAACTCCAGCATCGACGTGGACAGCCTGATGAGGTCTGTG
AGCAGGGTGTTCAAGTTCATCGACATGCCAACCGAGGGCAAGCCTACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCTCCTTCTCAATCAGCCCTGGCCAGAGGGTG
GGCCTGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCTGCAG
CAGTGGAGGAAGGCCTTCGGCGTGATCCCACAGAAGGTGTTCATCTTCTCTGGAACCTTC
AGAAAGAACCTGGACCCCTACGAGCAGTGGAGCGACCAGGAGATCTGGAAGGTGGCCGAC
GAGGTGGGCCTGAGATCTGTGATCGAGCAGTTCCCTGGCAAGCTGGACTTCGTGCTGGTG
GACGGGGGCTGCGTGCTGAGCCACGGCCACAAGCAGCTGATGTGCCTGGCCAGATCTGTG
CTGAGCAAGGCCAAGATCCTGCTGCTGGACGAGCCCAGTGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACTCCATCCAGAAGCTGCTGAACGAGAGGAGCCTGTTCCGGCAGGCC
ATCAGCCCCTCCGACAGGGTGAAGCTGTTCCCCCACCGGAACAGCAGCAAGTGCAAGTCT
AAGCCCCAGATCGCCGCCCTGAAGGAGGAGACCGAGGAGGAGGTGCAGGACACCAGGCTG
TAG
(pARM1836)
SEQ ID NO: 8
ATGCAGAGGTCGCCTCTGGAGAAGGCCAGCGTGGTGTCCAAGCTGTTTTTCAGCTGGACC
AGACCAATCCTGAGGAAGGGCTACAGACAGCGCCTGGAGCTGTCAGACATCTACCAGATC
CCTTCTGTGGATTCTGCTGACAATCTGTCTGAGAAGCTGGAGAGAGAGTGGGATAGAGAG
CTGGCCAGCAAGAAGAATCCTAAGCTGATCAATGCCCTGCGGAGGTGCTTTTTCTGGAGA
TTTATGTTCTACGGAATCTTTCTGTACCTGGGGGAGGTGACCAAGGCCGTGCAGCCTCTG
CTGCTGGGAAGAATCATCGCCTCCTACGACCCCGATAACAAGGAGGAGCGCTCTATCGCC
ATCTACCTGGGCATCGGCCTGTGCCTGCTGTTTATCGTGAGGACACTGCTGCTGCACCCA
GCCATCTTTGGCCTGCACCACATCGGAATGCAGATGAGAATCGCCATGTTTAGCCTGATC
TACAAGAAGACCCTGAAGCTGTCAAGCAGGGTGCTGGATAAGATCAGTATCGGACAGCTG
GTGAGTCTGCTGTCCAACAACCTGAACAAGTTTGATGAGGGACTGGCCCTGGCCCACTTC
GTGTGGATCGCTCCTCTGCAGGTGGCCCTGCTGATGGGGCTGATCTGGGAGCTGCTGCAG
GCCTCTGCCTTCTGCGGCCTGGGCTTCCTGATCGTGCTGGCCCTGTTTCAGGCCGGGCTG
GGGAGAATGATGATGAAGTACAGAGATCAGAGAGCTGGGAAGATCAGCGAGAGACTGGTG
ATCACCTCAGAGATGATCGAGAATATCCAGTCTGTGAAGGCATACTGCTGGGAGGAGGCC
ATGGAGAAGATGATCGAGAACCTGAGACAGACAGAGCTGAAGCTGACCCGGAAGGCCGCC
TACGTGAGATACTTCAATAGCAGCGCCTTCTTCTTCTCAGGGTTCTTTGTGGTGTTTCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTTCCCTGGGCCGTGCAGACA
TGGTACGACTCTCTGGGAGCCATCAACAAGATCCAGGATTTCCTGCAGAAGCAGGAGTAC
AAGACACTGGAGTACAACCTGACCACCACAGAGGTGGTGATGGAGAATGTGACAGCCTTC
ATGGGAGGAGGGATTTGGGGAGCTGTTTGAGAAGGCCAAGCAGAACATAACAATAGAAAG
ACCTCTAATGGCGATGACAGCCTGTTCTTCAGTAATTTCAGCCTGCTGGGCACCCCTGTG
CTGAAGGATATCAATTTCAAGATCGAGAGAGGACAGCTGCTGGCCGTGGCCGGATCCACC
GGAGCCGGCAAGACCTCACTGCTGATGGTGATCATGGGAGAGCTGGAGCCTTCAGAGGGC
AAGATCAAGCACAGTGGAAGAATCTCATTCTGCTCTCAGTTTTCCTGGATCATGCCTGGC
ACCATCAAGGAGAATATCATCTTTGGTGTGTCCTACGATGAGTACAGATACAGAAGCGTG
ATCAAGGCCTGCCAGCTGGAGGAGGACATCTCCAAGTTTGCAGAGAAGGACAATATCGTG
CTGGGAGAGGGTGGCATCACACTGAGCGGAGGCCAGAGGGCCAGAATCTCTCTGGCAAGA
GCAGTGTACAAGGATGCTGATCTGTACCTGCTGGACTCTCCTTTTGGATACCTGGATGTG
CTGACAGAGAAGGAGATCTTTGAGAGCTGCGTGTGCAAGCTGATGGCCAACAAGACCAGG
ATCCTGGTGACCTCTAAGATGGAGCACCTGAAGAAGGCTGACAAGATCCTGATCCTGCAC
GAGGGCAGCAGCTACTTTTACGGGACATTTAGCGAGCTGCAGAATCTGCAGCCAGACTTT
AGCAGCAAGCTGATGGGCTGCGATTCTTTCGACCAGTTTAGCGCCGAGAGAAGAAATAGC
ATCCTGACCGAGACCCTGCACAGGTTCAGCCTGGAGGGCGATGCCCCTGTGTCCTGGACA
GAGACAAAGAAGCAGTCTTTTAAGCAGACCGGAGAGTTTGGGGAGAAGAGGAAGAATTCT
ATCCTGAATCCAATCAACTCTATCAGGAAGTTTTCCATCGTGCAGAAGACCCCCCTGCAG
ATGAATGGCATCGAGGAGGATTCTGATGAGCCTCTGGAGAGAAGGCTGTCCCTGGTGCCA
GATTCTGAGCAGGGCGAGGCCATCCTGCCTCGCATCAGCGTGATCAGCACCGGCCCCACC
CTGCAGGCCAGGAGGAGGCAGTCTGTGCTGAACCTGATGACACACAGCGTGAACCAGGGC
CAGAACATCCACAGGAAGACAACAGCCTCCACAAGGAAGGTGAGCCTGGCCCCTCAGGCC
AACCTGACCGAGCTGGATATCTACAGCAGAAGGCTGTCTCAGGAGACCGGCCTGGAGATC
AGTGAGGAGATCAACGAGGAGGACCTGAAGGAGTGCTTTTTTGATGATATGGAGAGCATC
CCAGCCGTGACCACATGGAACACATACCTGAGGTACATCACCGTGCACAAGAGCCTGATC
TTTGTGCTGATCTGGTGCCTGGTGATCTTTCTGGCCGAGGTGGCCGCCTCTCTGGTGGTG
CTGTGGCTGCTGGGCAACACCCCTCTGCAGGACAAGGGGAATAGTACCCACAGCAGAAAT
AACAGCTACGCCGTGATCATCACCAGCACCAGTAGCTACTACGTGTTTTACATCTACGTG
GGAGTGGCCGACACCCTGCTGGCCATGGGCTTCTTCAGAGGTCTGCCACTGGTGCACACC
CTGATCACAGTGAGCAAGATCCTGCACCACAAGATGCTGCACTCTGTGCTGCAGGCCCCT
ATGAGCACCCTGAACACCCTGAAGGCCGGTGGGATCCTGAATAGATTCTCCAAGGATATC
GCCATCCTGGATGACCTGCTGCCTCTGACCATCTTTGACTTCATCCAGCTGCTGCTGATC
GTGATCGGAGCCATCGCCGTGGTGGCCGTGCTGCAGCCCTACATCTTTGTGGCCACAGTG
CCAGTGATCGTGGCCTTTATCATGCTGAGAGCCTACTTCCTGCAGACCAGCCAGCAGCTG
AAGCAGCTGGAGTCTGAGGGCAGGAGTCCAATCTTCACCCACCTGGTGACAAGCCTGAAG
GGACTGTGGACACTGAGGGCCTTCGGCCGGCAGCCTTACTTTGAGACCCTGTTCCACAAG
GCTCTGAATCTGCACACCGCCAACTGGTTCCTGTACCTGAGCACACTGCGCTGGTTCCAG
ATGAGAATCGAGATGATCTTTGTGATCTTCTTCATCGCCGTGACCTTCATCTCCATCCTG
ACAACAGGCGAGGGAGAGGGAAGAGTGGGCATCATCCTGACCCTGGCCATGAATATCATG
AGCACACTGCAGTGGGCTGTGAACTCCAGCATCGATGTGGATAGCCTGATGAGGTCTGTG
AGCAGGGTGTTTAAGTTCATCGACATGCCAACAGAGGGCAAGCCTACCAAGAGCACCAAG
CCATACAAGAATGGCCAGCTGAGCAAGGTGATGATCATCGAGAATAGCCACGTGAAGAAG
GATGACATCTGGCCCAGCGGGGGCCAGATGACCGTGAAGGATCTGACAGCCAAGTACACA
GAGGGCGGCAATGCCATCCTGGAGAACATCTCCTTCTCAATCAGCCCTGGCCAGAGGGTG
GGCCTGCTGGGAAGAACCGGCTCAGGGAAGAGTACCCTGCTGAGCGCCTTTCTGAGACTG
CTGAACACCGAGGGCGAGATCCAGATCGATGGCGTGTCTTGGGATTCAATCACCCTGCAG
CAGTGGAGGAAGGCCTTTGGCGTGATCCCACAGAAGGTGTTTATCTTTTCTGGAACATTT
AGAAAGAACCTGGATCCCTACGAGCAGTGGAGCGATCAGGAGATCTGGAAGGTGGCCGAT
GAGGTGGGGCTGAGATCTGTGATCGAGCAGTTTCCTGGGAAGCTGGACTTTGTGCTGGTG
GATGGGGGCTGCGTGCTGAGCCACGGCCACAAGCAGCTGATGTGCCTGGCCAGATCTGTG
CTGAGTAAGGCCAAGATCCTGCTGCTGGATGAGCCCAGTGCCCACCTGGATCCAGTGACA
TACCAGATCATCAGAAGAACCCTGAAGCAGGCCTTTGCCGATTGCACAGTGATCCTGTGC
GAGCACAGGATCGAGGCCATGCTGGAGTGCCAGCAGTTTCTGGTGATCGAGGAGAACAAG
GTGCGGCAGTACGATTCCATCCAGAAGCTGCTGAACGAGAGGAGCCTGTTCCGGCAGGCC
ATCAGCCCCTCCGACAGGGTGAAGCTGTTTCCCCACCGGAACAGCAGCAAGTGCAAGTCT
AAGCCCCAGATCGCCGCCCTGAAGGAGGAGACAGAGGAGGAGGTGCAGGATACAAGGCTG
TAG
(pARM1880)
SEQ ID NO: 9
ATGGGCCAGCGCAGCCCCCTCGAGAAGGCCAGCGTGGTGAGCAAGCTGTTCTTCAGCTGG
ACCCGCCCCATCCTGCGCAAGGGCTACCGCCAGCGCCTGGAGCTGAGCGACATCTACCAG
ATCCCCAGCGTGGACAGCGCCGACAACCTGAGCGAGAAGCTGGAGCGCGAGTGGGACCGC
GAGCTGGCCAGCAAGAAGAACCCCAAGCTGATCAACGCCCTGCGCCGCTGCTTCTTCTGG
CGCTTCATGTTCTACGGCATCTTCCTGTACCTGGGCGAGGTGACCAAGGCCGTGCAGCCC
CTGCTGCTGGGCCGCATCATCGCCAGCTACGACCCCGACAACAAGGAGGAGCGCAGCATC
GCCATCTACCTGGGCATCGGCCTGTGCCTGCTGTTCATCGTGCGCACCCTGCTGCTGCAC
CCCGCCATCTTCGGCCTGCACCACATCGGCATGCAGATGCGCATCGCCATGTTCAGCCTG
ATCTACAAGAAGACCCTGAAGCTGAGCAGCCGCGTGCTGGACAAGATCAGCATCGGCCAG
CTGGTGAGCCTGCTGAGCAACAACCTGAACAAGTTCGACGAGGGCCTGGCCCTGGCCCAC
TTCGTGTGGATCGCCCCCCTGCAGGTGGCCCTGCTGATGGGCCTGATCTGGGAGCTGCTG
CAGGCCAGCGCCTTCTGCGGCCTGGGCTTCCTGATCGTGCTGGCCCTGTTCCAGGCCGGC
CTGGGCCGCATGATGATGAAGTACCGCGACCAGCGCGCCGGCAAGATCAGCGAGCGCCTG
GTGATCACCAGCGAGATGATCGAGAACATCCAGAGCGTGAAGGCCTACTGCTGGGAGGAG
GCCATGGAGAAGATGATCGAGAACCTGCGCCAGACCGAGCTGAAGCTGACCCGCAAGGCC
GCCTACGTGCGCTACTTCAACAGCAGCGCCTTCTTCTTCAGCGGCTTCTTCGTGGTGTTC
CTGAGCGTGCTGCCCTACGCCCTGATCAAGGGCATCATCCTGCGCAAGATCTTCACCACC
ATCAGCTTCTGCATCGTGCTGCGCATGGCCGTGACCCGCCAGTTCCCCTGGGCCGTGCAG
ACCTGGTACGACAGCCTGGGCGCCATCAACAAGATCCAGGACTTCCTGCAGAAGCAGGAG
TACAAGACCCTGGAGTACAACCTGACCACCACCGAGGTGGTGATGGAGAACGTGACCGCC
TTCTGGGAGGAGGGCTTCGGCGAGCTGTTCGAGAAGGCCAAGCAGAACAACAACAACCGC
AAGACCAGCAACGGCGACGACAGCCTGTTCTTCAGCAACTTCAGCCTGCTGGGCACCCCC
GTGCTGAAGGACATCAACTTCAAGATCGAGCGCGGCCAGCTGCTGGCCGTGGCCGGCAGC
ACCGGCGCCGGCAAGACCAGCCTGCTGATGGTGATCATGGGCGAGCTGGAGCCCAGCGAG
GGCAAGATCAAGCACAGCGGCCGCATCAGCTTCTGCAGCCAGTTCAGCTGGATCATGCCC
GGCACCATCAAGGAGAACATCATCTTCGGCGTGAGCTACGACGAGTACCGCTACCGCAGC
GTGATCAAGGCCTGCCAGCTGGAGGAGGACATCAGCAAGTTCGCCGAGAAGGACAACATC
GTGCTGGGCGAGGGCGGCATCACCCTGAGCGGCGGCCAGCGCGCCCGCATCAGCCTGGCC
CGCGCCGTGTACAAGGACGCCGACCTGTACCTGCTGGACAGCCCCTTCGGCTACCTGGAC
GTGCTGACCGAGAAGGAGATCTTCGAGAGCTGCGTGTGCAAGCTGATGGCCAACAAGACC
CGCATCCTGGTGACCAGCAAGATGGAGCACCTGAAGAAGGCCGACAAGATCCTGATCCTG
CACGAGGGCAGCAGCTACTTCTACGGCACCTTCAGCGAGCTGCAGAACCTGCAGCCCGAC
TTCAGCAGCAAGCTGATGGGCTGCGACAGCTTCGACCAGTTCAGCGCCGAGCGCCGCAAC
AGCATCCTGACCGAGACCCTGCACCGCTTCAGCCTGGAGGGCGACGCCCCCGTGAGCTGG
ACCGAGACCAAGAAGCAGAGCTTCAAGCAGACCGGCGAGTTCGGCGAGAAGCGCAAGAAC
AGCATCCTGAACCCCATCAACAGCATCCGCAAGTTCAGCATCGTGCAGAAGACCCCCCTG
CAGATGAACGGCATCGAGGAGGACAGCGACGAGCCCCTGGAGCGCCGCCTGAGCCTGGTG
CCCGACAGCGAGCAGGGCGAGGCCATCCTGCCCCGCATCAGCGTGATCAGCACCGGCCCC
ACCCTGCAGGCCCGCCGCCGCCAGAGCGTGCTGAACCTGATGACCCACAGCGTGAACCAG
GGCCAGAACATCCACCGCAAGACCACCGCCAGCACCCGCAAAGTGAGCCTGGCCCCCCAG
GCCAACCTGACCGAGCTGGACATCTACAGCCGCCGCCTGAGCCAGGAGACCGGCCTGGAG
ATCAGCGAGGAGATCAACGAGGAGGACCTGAAGGAGTGCTTCTTCGACGACATGGAGAGC
ATCCCCGCCGTGACCACCTGGAACACCTACCTGCGCTACATCACCGTGCACAAGAGCCTG
ATCTTCGTGCTGATCTGGTGCCTGGTGATCTTCCTGGCCGAGGTGGCCGCCAGCCTGGTG
GTGCTGTGGCTGCTGGGCAACACCCCCCTGCAGGACAAGGGCAACAGCACCCACAGCCGC
AACAACAGCTACGCCGTGATCATCACCAGCACCAGCAGCTACTACGTGTTCTACATCTAC
GTGGGCGTGGCCGACACCCTGCTGGCCATGGGCTTCTTCCGCGGCCTGCCCCTGGTGCAC
ACCCTGATCACCGTGAGCAAGATCCTGCACCACAAGATGCTGCACAGCGTGCTGCAGGCC
CCCATGAGCACCCTGAACACCCTGAAGGCCGGCGGCATCCTGAACCGCTTCAGCAAGGAC
ATCGCCATCCTGGACGACCTGCTGCCCCTGACCATCTTCGACTTCATCCAGCTGCTGCTG
ATCGTGATCGGCGCCATCGCCGTGGTGGCCGTGCTGCAGCCCTACATCTTCGTGGCCACC
GTGCCCGTGATCGTGGCCTTCATCATGCTGCGCGCCTACTTCCTGCAGACCAGCCAGCAG
CTGAAGCAGCTGGAGAGCGAGGGCCGCAGCCCCATCTTCACCCACCTGGTGACCAGCCTG
AAGGGCCTGTGGACCCTGCGCGCCTTCGGCCGCCAGCCCTACTTCGAGACCCTGTTCCAC
AAGGCCCTGAACCTGCACACCGCCAACTGGTTCCTGTACCTGAGCACCCTGCGCTGGTTC
CAGATGCGCATCGAGATGATCTTCGTGATCTTCTTCATCGCCGTGACCTTCATCAGCATC
CTGACCACCGGCGAGGGCGAGGGCCGCGTGGGCATCATCCTGACCCTGGCCATGAACATC
ATGAGCACCCTGCAGTGGGCCGTGAACAGCAGCATCGACGTGGACAGCCTGATGCGCAGC
GTGAGCCGCGTGTTCAAGTTCATCGACATGCCCACCGAGGGCAAGCCCACCAAGAGCACC
AAGCCCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAAGTAC
ACCGAGGGCGGCAACGCCATCCTGGAGAACATCAGCTTCAGCATCAGCCCCGGCCAGCGC
GTGGGCCTGCTGGGCCGCACCGGCAGCGGCAAGAGCACCCTGCTGAGCGCCTTCCTGCGC
CTGCTGAACACCGAGGGCGAGATCCAGATCGACGGCGTGAGCTGGGACAGCATCACCCTG
CAGCAGTGGCGCAAGGCCTTCGGCGTGATCCCCCAGAAGGTGTTCATCTTCAGCGGCACC
TTCCGCAAGAACCTGGACCCCTACGAGCAGTGGAGCGACCAGGAGATCTGGAAGGTGGCC
GACGAGGTGGGCCTGCGCAGCGTGATCGAGCAGTTCCCCGGCAAGCTGGACTTCGTGCTG
GTGGACGGCGGCTGCGTGCTGAGCCACGGCCACAAGCAGCTGATGTGCCTGGCCCGCAGC
GTGCTGAGCAAGGCCAAGATCCTGCTGCTGGACGAGCCCAGCGCCCACCTGGACCCCGTG
ACCTACCAGATCATCCGCCGCACCCTGAAGCAGGCCTTCGCCGACTGCACCGTGATCCTG
TGCGAGCACCGCATCGAGGCCATGCTGGAGTGCCAGCAGTTCCTGGTGATCGAGGAGAAC
AAGGTGCGCCAGTACGACAGCATCCAGAAGCTGCTGAACGAGCGCAGCCTGTTCCGCCAG
GCCATCAGCCCCAGCGACCGCGTGAAGCTTTTCCCCCACCGCAACAGCAGCAAGTGCAAG
AGCAAGCCCCAGATCGCCGCCCTGAAGGAGGAGACCGAGGAGGAGGTGCAGGACACCCGC
CTGTAG
(pARM1947)
SEQ ID NO: 10
ATGCAGAGGAGCCCCCTGGAGAAGGCTAGCGTGGTGAGCAAGCTGTTCTTCAGCTGGACC
AGGCCCATCCTGAGGAAGGGCTACAGGCAGAGGCTGGAGCTGAGCGACATCTACCAGATC
CCCAGCGTGGACAGCGCCGACAACCTGAGCGAGAAGCTGGAGAGGGAGTGGGACAGGGAG
CTGGCCAGCAAGAAGAACCCCAAGCTGATCAACGCCCTGCGGAGGTGCTTCTTCTGGAGG
TTCATGTTCTACGGCATCTTCCTGTACCTGGGCGAGGTGACCAAGGCCGTGCAGCCCCTG
CTGCTGGGCAGGATCATCGCCAGCTACGACCCCGACAACAAGGAGGAGAGGAGCATCGCC
ATCTACCTGGGCATCGGCCTGTGCCTGCTGTTCATCGTGAGGACCCTGCTGCTGCACCCC
GCCATCTTCGGCCTGCACCACATCGGCATGCAGATGAGGATCGCCATGTTCAGCCTGATC
TACAAGAAGACCCTGAAGCTGAGCAGCAGGGTGCTGGACAAGATCAGCATCGGCCAGCTG
GTGAGCCTGCTGAGCAACAACCTGAACAAGTTCGACGAGGGCCTGGCCCTGGCCCACTTC
GTGTGGATCGCCCCCCTGCAGGTGGCCCTGCTGATGGGCCTGATCTGGGAGCTGCTGCAG
GCCAGCGCCTTCTGCGGCCTGGGCTTCCTGATCGTGCTGGCCCTGTTCCAGGCCGGCCTG
GGCAGGATGATGATGAAGTACAGGGACCAGAGGGCCGGCAAGATCAGCGAGAGGCTGGTG
ATCACCAGCGAGATGATCGAGAACATCCAGAGCGTGAAGGCCTACTGCTGGGAGGAGGCC
ATGGAGAAGATGATCGAGAACCTGAGGCAGACCGAGCTGAAGCTGACCCGGAAGGCCGCC
TACGTGAGGTACTTCAACAGCAGCGCCTTCTTCTTCAGCGGCTTCTTCGTGGTGTTCCTG
AGCGTGCTGCCCTACGCCCTGATCAAGGGCATCATCCTGCGGAAGATCTTCACCACCATC
AGCTTCTGCATCGTGCTGAGGATGGCCGTGACCCGGCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGCTTCGGCGAGCTGTTCGAGAAGGCCAAGCAGAACAACAACAACAGGAAG
ACCAGCAACGGCGACGACAGCCTGTTCTTCAGCAACTTCAGCCTGCTGGGCACCCCCGTG
CTGAAGGACATCAACTTCAAGATCGAGAGGGGCCAGCTGCTGGCCGTGGCCGGCAGCACC
GGCGCCGGCAAGACCAGCCTGCTGATGGTGATCATGGGCGAGCTGGAGCCCAGCGAGGGC
AAGATCAAGCACAGCGGCAGGATCAGCTTCTGCAGCCAGTTCAGCTGGATCATGCCCGGC
ACCATCAAGGAGAACATCATCTTCGGCGTGAGCTACGACGAGTACAGGTACAGGAGCGTG
ATCAAGGCCTGCCAGCTGGAGGAGGACATCAGCAAGTTCGCCGAGAAGGACAACATCGTG
CTGGGCGAGGGCGGCATCACCCTGAGCGGCGGCCAGAGGGCCAGGATCAGCCTGGCCAGG
GCCGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGAGGAGGAACAGC
ATCCTGACCGAGACACTGCACAGGTTCAGCCTGGAGGGCGACGCCCCCGTGAGCTGGACC
GAGACAAAGAAGCAGAGCTTCAAGCAGACCGGCGAGTTCGGCGAGAAGAGGAAGAACAGC
ATCCTGAACCCCATCAACAGCATCAGGAAGTTCAGCATCGTGCAGAAGACCCCCCTGCAG
ATGAACGGCATCGAGGAGGACAGCGACGAGCCCCTGGAGAGGAGGCTGAGCCTGGTGCCC
GACAGCGAGCAGGGCGAGGCCATCCTGCCCAGGATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCCCAGGCC
AACCTGACCGAGCTGGACATCTACAGCAGGAGGCTGAGCCAGGAGACAGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCCCTGCAGGACAAGGGCAACAGCACCCACAGCAGGAAC
AACAGCTACGCCGTGATCATCACCAGCACCAGCAGCTACTACGTGTTCTACATCTACGTG
GGCGTGGCCGACACCCTGCTGGCCATGGGCTTCTTCAGGGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGCATCCTGAACAGGTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCCGTGATCGTGGCCTTCATCATGCTGAGGGCCTACTTCCTGCAGACCAGCCAGCAGCTG
AAGCAGCTGGAGAGCGAGGGCAGGAGCCCCATCTTCACCCACCTGGTGACCAGCCTGAAG
GGCCTGTGGACCCTGAGGGCCTTCGGCCGGCAGCCCTACTTCGAGACACTGTTCCACAAG
GCCCTGAACCTGCACACCGCCAACTGGTTCCTGTACCTGAGCACCCTGAGGTGGTTCCAG
ATGAGGATCGAGATGATCTTCGTGATCTTCTTCATCGCCGTGACCTTCATCAGCATCCTG
ACCACCGGCGAGGGCGAGGGCAGGGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCCACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGCAGGACCGGCAGCGGCAAGAGCACCCTGCTGAGCGCCTTCCTGAGGCTG
CTGAACACCGAGGGCGAGATCCAGATCGACGGCGTGAGCTGGGACAGCATCACCCTGCAG
CAGTGGAGGAAGGCCTTCGGCGTGATCCCCCAGAAGGTGTTCATCTTCAGCGGCACCTTC
AGGAAGAACCTGGACCCCTACGAGCAGTGGAGCGACCAGGAGATCTGGAAGGTGGCCGAC
GAGGTGGGCCTGAGGAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCAGGAGCGTG
CTGAGCAAGGCCAAGATCCTGCTGCTGGACGAGCCCAGCGCCCACCTGGACCCCGTGACC
TACCAGATCATCAGGAGGACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACAGAGGAGGAGGTGCAGGACACCAGGCTG
TAG
(pARM1948)
SEQ ID NO: 11
ATGCAGAGGTCCCCCTTGGAAAAAGCCTCCGTGGTGTCTAAATTGTTCTTCTCCTGGACA
AGGCCCATATTGAGGAAAGGATACAGGCAGAGGTTGGAATTGTCCGACATATACCAGATA
CCCTCCGTGGACTCCGCCGACAACTTGTCCGAAAAATTGGAAAGGGAATGGGATAGGGAA
TTGGCCTCCAAAAAAAACCCCAAATTGATAAACGCCTTGAGGAGGTGCTTCTTCTGGAGG
TTCATGTTCTACGGAATATTCTTGTACTTGGGAGAAGTGACAAAAGCCGTGCAGCCCTTG
TTGTTGGGAAGGATAATAGCCTCCTACGACCCCGACAACAAAGAAGAAAGGTCCATAGCC
ATATACTTGGGAATAGGATTGTGCTTGTTGTTCATAGTGAGGACATTGTTGTTGCACCCC
GCCATATTCGGATTGCACCACATAGGAATGCAGATGAGGATAGCCATGTTCTCCTTGATA
TACAAAAAAACATTGAAATTGTCCTCCAGGGTGTTGGACAAAATATCCATAGGACAGTTG
GTGTCCTTGTTGTCCAACAACTTGAACAAATTCGACGAAGGATTGGCCTTGGCCCACTTC
GTGTGGATAGCCCCCTTGCAGGTGGCCTTGTTGATGGGATTGATATGGGAATTGTTGCAG
GCCTCCGCCTTCTGCGGATTGGGATTCTTGATAGTGTTGGCCTTGTTCCAGGCCGGATTG
GGAAGGATGATGATGAAATATAGGGACCAGAGGGCCGGAAAAATATCCGAAAGGTTGGTG
ATAACATCCGAAATGATAGAAAACATACAGTCCGTGAAAGCCTACTGCTGGGAAGAAGCC
ATGGAAAAAATGATAGAAAACTTGAGGCAGACAGAATTGAAATTGACAAGGAAAGCCGCC
TACGTGAGGTACTTCAACTCCTCCGCCTTCTTCTTCTCCGGATTCTTCGTGGTGTTCTTG
TCCGTGTTGCCCTACGCCTTGATAAAAGGAATAATATTGAGGAAAATATTCACAACAATA
TCCTTCTGCATAGTGTTGAGGATGGCCGTGACAAGGCAGTTCCCCTGGGCCGTGCAGACA
TGGTATGACTCCTTGGGAGCCATAAACAAAATACAGGACTTCTTGCAGAAACAGGAATAC
AAAACATTGGAATACAACTTGACAACAACAGAAGTGGTGATGGAAAACGTGACAGCCTTC
TGGGAAGAAGGATTCGGAGAATTGTTCGAAAAAGCCAAACAGAACAACAACAACAGGAAA
ACATCCAACGGAGACGACTCCTTGTTCTTCTCCAACTTCTCCTTGTTGGGAACACCCGTG
TTGAAAGACATAAACTTCAAAATAGAAAGGGGACAGTTGTTGGCCGTGGCCGGATCCACA
GGAGCCGGAAAAACATCCTTGTTGATGGTGATAATGGGAGAATTGGAACCCTCCGAAGGA
AAAATAAAACACTCCGGAAGGATATCCTTCTGCTCCCAGTTCTCCTGGATAATGCCCGGA
ACAATAAAAGAAAACATAATATTCGGAGTGTCCTACGACGAATACAGGTACAGGTCCGTG
ATAAAAGCCTGCCAGTTGGAAGAAGACATATCCAAATTCGCCGAAAAAGACAACATAGTG
TTGGGAGAAGGAGGAATAACATTGTCCGGAGGACAGAGGGCCAGGATATCCTTGGCCAGG
GCCGTGTACAAAGACGCCGACTTGTACTTGTTGGACTCCCCCTTCGGATACTTGGACGTG
TTGACAGAAAAAGAAATATTCGAATCCTGCGTGTGCAAATTGATGGCCAACAAAACAAGG
ATATTGGTGACATCCAAAATGGAACACTTGAAAAAAGCCGACAAAATATTGATATTGCAC
GAAGGATCCTCCTACTTCTACGGAACATTCTCCGAATTGCAGAACTTGCAGCCCGACTTC
TCCTCCAAATTGATGGGATGCGACTCCTTTGACCAGTTCTCCGCCGAAAGGAGGAACTCC
ATATTGACAGAAACATTGCACAGGTTCTCCTTGGAAGGAGACGCCCCCGTGTCCTGGACA
GAAACAAAAAAACAGTCCTTCAAACAGACAGGAGAATTCGGAGAAAAAAGGAAAAACTCC
ATATTGAACCCCATAAACTCCATAAGGAAATTCTCCATAGTGCAGAAAACACCCTTGCAG
ATGAACGGAATAGAAGAAGACTCCGACGAACCCTTGGAAAGGAGGTTGTCCTTGGTGCCC
GACTCCGAACAGGGAGAAGCCATATTGCCCAGGATATCCGTGATATCCACAGGACCCACA
TTGCAGGCCAGGAGGAGGCAGTCCGTGTTGAACTTGATGACACACTCCGTGAACCAGGGA
CAGAACATACACAGGAAAACAACAGCCTCCACAAGGAAAGTGTCCTTGGCCCCCCAGGCC
AACTTGACAGAATTGGACATATACTCCAGGAGGTTGTCCCAGGAAACAGGATTGGAAATA
TCCGAAGAAATAAACGAAGAAGACTTGAAAGAATGCTTCTTCGATGACATGGAATCCATA
CCCGCCGTGACAACATGGAACACATACTTGAGGTACATAACAGTGCATAAATCCTTGATA
TTCGTGTTGATATGGTGCTTGGTGATATTCTTGGCTGAAGTGGCCGCCTCCTTGGTGGTG
TTGTGGTTGTTGGGAAACACACCCTTGCAGGACAAAGGAAACTCCACACACTCCTCCAAC
AACTCCTACGCCGTGATAATAACATCCACATCCTCCTACTACGTGTTCTACATATACGTG
GGAGTGGCCGACACATTGTTGGCCATGGGATTCTTCAGGGGATTGCCCTTGGTGCACACA
TTGATAACAGTGTCCAAAATATTGCACCACAAAATGTTGCACTCCGTGTTGCAGGCCCCC
ATGTCCACATTGAACACATTGAAAGCCGGAGGAATATTGAACAGGTTCTCCAAAGACATA
GCCATATTGGACGACTTGTTGCCCTTGACAATATTCGACTTCATACAGTTGTTGTTGATA
GTGATAGGAGCCATAGCCGTGGTGGCCGTGTTGCAGCCCTACATATTCGTGGCCACAGTG
CCCGTGATAGTGGCCTTCATAATGTTGAGGGCCTACTTCTTGCAGACATCCCAGCAGTTG
AAACAGTTGGAATCCGAAGGAAGGTCCCCCATATTCACACACTTGGTGACATCCTTGAAA
GGATTGTGGACATTGAGGGCCTTCGGAAGGCAGCCCTACTTCGAAACATTGTTCCACAAA
GCCTTGAACTTGCACACAGCCAACTGGTTCTTGTACTTGTCCACATTGAGGTGGTTCCAG
ATGAGGATAGAAATGATATTCGTGATATTCTTCATAGCCGTGACATTCATATCCATATTG
ACAACAGGAGAAGGAGAAGGAAGGGTGGGAATAATATTGACATTGGCCATGAACATAATG
TCCACATTGCAGTGGGCCGTGAACTCCTCCATAGACGTGGACTCCTTGATGAGGTCCGTG
TCCAGGGTGTTCAAATTCATAGACATGCCCACAGAAGGAAAACCCACAAAATCCACAAAA
CCCTACAAAAACGGACAGTTGTCCAAAGTGATGATAATAGAAAACTCCCACGTGAAAAAA
GACGACATATGGCCCTCCGGAGGACAGATGACAGTGAAAGACTTGACAGCCAAATACACA
GAAGGAGGAAACGCCATATTGGAAAACATATCCTTCTCCATATCCCCCGGACAGAGGGTG
GGATTGTTGGGAAGGACAGGATCCGGAAAATCCACATTGTTGTCCGCCTTCTTGAGGTTG
TTGAACACAGAAGGAGAAATACAGATAGACGGAGTGTCCTGGGACTCCATAACATTGCAG
CAGTGGAGGAAAGCCTTCGGAGTGATACCCCAGAAAGTGTTCATATTCTCCGGAACATTC
AGGAAAAACTTGGACCCCTACGAACAGTGGTCCGACCAGGAAATATGGAAAGTGGCCGAC
GAAGTGGGATTGAGGTCCGTGATAGAACAGTTCCCCGGAAAATTGGACTTCGTGTTGGTG
GACGGAGGATGCGTGTTGTCCCACGGACACAAACAGTTGATGTGCTTGGCCAGGTCCGTG
TTGTCCAAAGCCAAAATATTGTTGTTGGACGAACCCTCCGCCCACTTGGACCCCGTGACA
TACCAGATAATAAGGAGGACATTGAAACAGGCCTTCGCCGACTGCACAGTGATATTGTGC
GAACACAGGATAGAAGCCATGTTGGAATGCCAGCAGTTCTTGGTGATAGAAGAAAACAAA
GTGAGGCAGTACGACTCCATACAGAAATTGTTGAACGAAAGGTCCTTGTTCAGGCAGGCC
ATATCCCCCTCCGACAGGGTGAAATTGTTCCCCCACAGGAACTCCTCCAAATGCAAATCC
AAACCCCAGATAGCCGCCTTGAAAGAAGAAACAGAAGAAGAAGTGCAGGACACAAGGTTG
TAG
(pARM2047)
SEQ ID NO: 12
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2048)
SEQ ID NO: 13
ATGCAGAGGAGCCCCCTGGAGAAGGCTAGCGTGGTCAGCAAGCTGTTCTTCAGCTGGACC
AGACCAATCCTGAGGAAAGGATACAGACAGCGCCTGGAACTGAGCGACATATACCAAATC
CCCAGCGTGGACAGCGCCGACAACCTAAGCGAGAAGCTGGAAAGAGAATGGGATAGGGAG
CTGGCCAGCAAGAAGAACCCCAAGCTCATCAACGCCCTGCGGCGATGCTTCTTCTGGAGG
TTCATGTTCTACGGAATCTTCCTGTACCTGGGGGAGGTCACCAAGGCAGTACAGCCCCTC
CTGCTGGGCAGAATCATAGCCAGCTACGACCCCGACAACAAGGAGGAACGCAGCATCGCG
ATCTACCTGGGCATCGGCCTGTGCCTGCTGTTCATCGTGAGGACACTGCTCCTACACCCC
GCCATCTTCGGCCTGCACCACATCGGAATGCAGATGAGAATCGCCATGTTCAGCCTGATC
TACAAGAAGACCCTGAAGCTGAGCAGCAGGGTGCTAGACAAGATCAGCATCGGACAGCTG
GTGAGCCTGCTGAGCAACAACCTGAACAAGTTCGACGAAGGACTGGCACTGGCACACTTC
GTGTGGATCGCCCCACTGCAAGTGGCACTCCTGATGGGGCTGATCTGGGAGCTGCTGCAG
GCGAGCGCCTTCTGCGGACTGGGCTTCCTGATAGTCCTGGCCCTGTTCCAGGCCGGGCTA
GGGAGAATGATGATGAAGTACAGAGACCAGAGGGCCGGGAAGATCAGCGAGAGACTCGTG
ATCACCAGCGAGATGATCGAGAACATCCAGAGCGTGAAGGCATACTGCTGGGAAGAGGCA
ATGGAGAAGATGATCGAGAACCTGAGACAGACAGAGCTGAAGCTGACCCGGAAGGCAGCC
TACGTGAGATACTTCAACAGCAGCGCCTTCTTCTTCAGCGGGTTCTTCGTGGTCTTCCTG
AGCGTGCTGCCCTACGCACTAATCAAGGGAATCATCCTGCGGAAGATCTTCACAACCATC
AGCTTCTGCATCGTGCTGCGCATGGCGGTCACCCGGCAGTTCCCCTGGGCCGTACAGACA
TGGTACGACAGCCTGGGAGCCATCAACAAGATACAGGACTTCCTGCAGAAGCAAGAGTAC
AAGACACTGGAGTACAACCTGACGACCACAGAAGTAGTGATGGAGAACGTAACCGCCTTC
TGGGAGGAGGGATTCGGGGAGCTGTTCGAGAAAGCAAAGCAGAACAACAACAACCGGAAG
ACCAGCAACGGCGACGACAGCCTCTTCTTCAGCAACTTCAGCCTGCTGGGCACCCCCGTC
CTGAAGGACATCAACTTCAAGATAGAGAGGGGACAGCTGCTGGCGGTGGCCGGAAGCACC
GGAGCAGGCAAGACCAGCCTGCTAATGGTGATCATGGGAGAACTGGAGCCCAGCGAGGGC
AAGATCAAGCACAGCGGAAGGATCAGCTTCTGCAGCCAGTTCAGCTGGATCATGCCCGGC
ACCATCAAGGAGAACATCATCTTCGGCGTGAGCTACGACGAGTACCGCTACAGAAGCGTC
ATCAAGGCATGCCAACTAGAAGAGGACATCAGCAAGTTCGCAGAGAAGGACAACATAGTG
CTGGGAGAAGGCGGAATCACACTGAGCGGAGGCCAACGAGCAAGAATCAGCCTGGCAAGA
GCAGTATACAAGGACGCCGACCTGTACCTGCTGGACAGCCCCTTCGGATACCTAGACGTG
CTGACCGAGAAGGAGATATTCGAAAGCTGCGTCTGCAAGCTGATGGCCAACAAGACCAGG
ATCCTGGTCACCAGCAAGATGGAACACCTGAAGAAAGCCGACAAGATCCTGATCCTGCAC
GAAGGCAGCAGCTACTTCTACGGGACATTCAGCGAACTCCAGAACCTACAGCCAGACTTC
AGCAGCAAGCTCATGGGATGCGACAGCTTCGACCAGTTCAGCGCAGAGAGACGGAACAGC
ATCCTAACCGAGACACTGCACAGGTTCAGCCTGGAAGGAGACGCCCCCGTCAGCTGGACA
GAGACGAAGAAACAGAGCTTCAAACAGACCGGAGAGTTCGGGGAGAAACGCAAGAACAGC
ATCCTCAACCCAATCAACAGCATACGAAAGTTCAGCATCGTGCAGAAGACCCCACTGCAG
ATGAACGGCATCGAAGAGGACAGCGACGAGCCCCTGGAGAGAAGGCTGAGCCTGGTACCA
GACAGCGAGCAGGGAGAGGCGATACTGCCCCGCATCAGCGTGATCAGCACCGGCCCCACG
CTGCAGGCACGAAGGCGCCAGAGCGTCCTGAACCTGATGACACACAGCGTGAACCAAGGC
CAGAACATCCACCGAAAGACAACCGCAAGCACAAGGAAGGTGAGCCTGGCCCCACAGGCA
AACCTGACCGAACTGGACATCTACAGCAGAAGGCTGAGCCAGGAGACCGGCCTGGAGATC
AGCGAAGAGATCAACGAAGAGGACCTGAAGGAGTGCTTCTTCGACGACATGGAGAGCATA
CCAGCAGTGACCACATGGAACACATACCTGAGGTACATCACCGTCCACAAGAGCCTGATC
TTCGTGCTAATCTGGTGCCTGGTGATCTTCCTGGCAGAGGTGGCCGCCAGCCTGGTGGTG
CTGTGGCTCCTGGGAAACACCCCACTGCAAGACAAAGGGAACAGCACCCACAGCAGGAAC
AACAGCTACGCAGTGATCATCACCAGCACCAGCAGCTACTACGTGTTCTACATCTACGTG
GGAGTAGCCGACACCCTGCTGGCCATGGGATTCTTCAGAGGCCTACCACTGGTGCACACC
CTAATCACAGTGAGCAAGATCCTGCACCACAAGATGCTGCACAGCGTGCTGCAAGCACCC
ATGAGCACCCTCAACACGCTGAAGGCAGGCGGGATCCTGAACAGGTTCAGCAAGGACATA
GCCATCCTGGACGACCTGCTGCCCCTGACCATCTTCGACTTCATCCAGCTGCTGCTGATC
GTGATCGGAGCCATAGCAGTGGTCGCAGTGCTGCAACCCTACATCTTCGTGGCAACAGTG
CCAGTGATAGTGGCCTTCATCATGCTGAGAGCATACTTCCTCCAAACCAGCCAGCAACTC
AAGCAGCTGGAAAGCGAAGGCAGGAGCCCAATCTTCACCCACCTGGTGACAAGCCTGAAG
GGACTCTGGACACTGAGGGCCTTCGGACGGCAGCCCTACTTCGAAACCCTGTTCCACAAA
GCCCTGAACCTGCACACCGCCAACTGGTTCCTGTACCTGAGCACACTGCGCTGGTTCCAA
ATGAGAATAGAAATGATCTTCGTCATCTTCTTCATCGCCGTGACCTTCATCAGCATCCTG
ACAACAGGAGAAGGAGAAGGAAGAGTGGGCATCATCCTGACCCTGGCCATGAACATCATG
AGCACACTGCAGTGGGCCGTGAACAGCAGCATAGACGTGGACAGCCTGATGCGAAGCGTG
AGCCGAGTCTTCAAGTTCATCGACATGCCCACCGAGGGCAAGCCCACCAAGAGCACCAAG
CCCTACAAGAACGGCCAACTCAGCAAGGTGATGATCATCGAGAACAGCCACGTGAAGAAA
GACGACATCTGGCCCAGCGGGGGCCAAATGACCGTCAAAGACCTCACAGCCAAGTACACA
GAAGGCGGAAACGCCATCCTGGAGAACATCAGCTTCAGCATCAGCCCCGGCCAGAGGGTG
GGCCTCCTGGGAAGAACCGGAAGCGGCAAGAGCACCCTGCTGAGCGCCTTCCTGAGACTA
CTGAACACCGAAGGCGAGATCCAGATCGACGGCGTGAGCTGGGACAGCATCACCCTGCAA
CAGTGGAGGAAGGCCTTCGGCGTGATACCACAGAAGGTGTTCATCTTCAGCGGAACCTTC
AGGAAGAACCTGGACCCCTACGAACAGTGGAGCGACCAGGAGATCTGGAAGGTGGCAGAC
GAGGTGGGGCTCAGAAGCGTGATAGAACAGTTCCCCGGGAAGCTGGACTTCGTCCTGGTG
GACGGGGGCTGCGTCCTAAGCCACGGCCACAAGCAGCTGATGTGCCTGGCCAGAAGCGTG
CTCAGCAAGGCGAAGATCCTGCTGCTGGACGAACCCAGCGCCCACCTGGACCCAGTAACA
TACCAGATCATCCGGAGAACCCTGAAGCAGGCATTCGCCGACTGCACAGTAATCCTCTGC
GAACACAGGATAGAAGCAATGCTGGAATGCCAACAGTTCCTGGTCATCGAAGAGAACAAG
GTGCGGCAGTACGACAGCATCCAGAAGCTGCTGAACGAGAGGAGCCTCTTCCGGCAAGCC
ATCAGCCCCAGCGACAGGGTGAAGCTGTTCCCCCACCGGAACAGCAGCAAGTGCAAGAGC
AAGCCCCAGATCGCCGCCCTGAAGGAAGAGACCGAGGAAGAGGTGCAGGACACCAGGCTG
TGA
(pARM2049)
SEQ ID NO: 14
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2088)
SEQ ID NO: 15
ATGCAGAGGAGCCCCCTGGAGAAGGCCAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCCGCGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGCGCGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGCGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGCGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACCGCTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCCATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGCGCGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCCCAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCCGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTTTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2089)
SEQ ID NO: 16
ATGCAGCGCAGCCCCCTGGAGAAGGCCAGCGTGGTGAGCAAGCTGTTCTTCAGCTGGACC
CGCCCCATCCTGCGCAAGGGCTACAGACAGCGCCTGGAGCTGAGCGACATCTACCAGATC
CCTTCTGTGGACAGCGCCGACAACCTGAGCGAGAAGCTGGAGCGCGAGTGGGACCGCGAG
CTGGCCAGCAAGAAGAACCCTAAGCTGATCAACGCCCTGCGCCGCTGCTTCTTCTGGCGC
TTCATGTTCTACGGCATCTTCCTGTACCTGGGGGAGGTGACCAAGGCCGTGCAGCCCCTG
CTGCTGGGCCGCATCATCGCCAGCTACGACCCCGACAACAAGGAGGAGCGCAGCATCGCC
ATCTACCTGGGCATCGGCCTGTGCCTGCTGTTCATCGTGCGCACCCTGCTGCTGCACCCC
GCCATCTTCGGCCTGCACCACATCGGCATGCAGATGCGCATCGCCATGTTCAGCCTGATC
TACAAGAAGACCCTGAAGCTGTCAAGCAGGGTGCTGGACAAGATCAGCATCGGCCAGCTG
GTGAGCCTGCTGAGCAACAACCTGAACAAGTTCGACGAGGGCCTGGCCCTGGCCCACTTC
GTGTGGATCGCTCCCCTGCAGGTGGCCCTGCTGATGGGCCTGATCTGGGAGCTGCTGCAG
GCCAGCGCCTTCTGCGGCCTGGGCTTCCTGATCGTGCTGGCCCTGTTCCAGGCCGGCCTG
GGCCGCATGATGATGAAGTACAGAGACCAGCGCGCCGGCAAGATCAGCGAGCGCCTGGTG
ATCACCAGCGAGATGATCGAGAACATCCAGAGCGTGAAGGCCTACTGCTGGGAGGAGGCC
ATGGAGAAGATGATCGAGAACCTGCGCCAGACCGAGCTGAAGCTGACCCGCAAGGCCGCC
TACGTGCGCTACTTCAACAGCAGCGCCTTCTTCTTCTCAGGCTTCTTCGTGGTGTTCCTG
AGCGTGCTGCCCTACGCCCTGATCAAGGGCATCATCCTGCGCAAGATCTTCACCACCATC
AGCTTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGCTTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCCGTG
CTGAAGGACATCAACTTCAAGATCGAGCGCGGCCAGCTGCTGGCCGTGGCCGGCAGCACC
GGCGCCGGCAAGACCTCACTGCTGATGGTGATCATGGGCGAGCTGGAGCCTAGCGAGGGC
AAGATCAAGCACAGCGGCCGCATCTCATTCTGCAGCCAGTTCAGCTGGATCATGCCCGGC
ACCATCAAGGAGAACATCATCTTCGGTGTGAGCTACGACGAGTACCGCTACCGCAGCGTG
ATCAAGGCCTGCCAGCTGGAGGAGGACATCAGCAAGTTCGCCGAGAAGGACAACATCGTG
CTGGGAGAGGGTGGCATCACCCTGAGCGGCGGCCAGAGGGCCCGCATCAGCCTGGCCCGC
GCCGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCCGC
ATCCTGGTGACCTCTAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGCGCCGCAACAGC
ATCCTGACCGAGACCCTGCACCGCTTCAGCCTGGAGGGCGACGCCCCCGTGAGCTGGACC
GAGACCAAGAAGCAGTCTTTCAAGCAGACCGGCGAGTTCGGCGAGAAGCGCAAGAACAGC
ATCCTGAACCCCATCAACAGCATCCGCAAGTTCAGCATCGTGCAGAAGACCCCACTGCAG
ATGAACGGCATCGAGGAGGACAGCGACGAGCCCCTGGAGCGCCGCCTGAGCCTGGTGCCC
GACAGCGAGCAGGGCGAGGCCATCCTGCCTCGCATCAGCGTGATCAGCACCGGCCCCACC
CTGCAGGCCCGCCGCCGCCAGAGCGTGCTGAACCTGATGACCCACAGCGTGAACCAGGGC
CAGAACATCCACAGGAAGACCACCGCCAGCACCAGGAAAGTGAGCCTGGCCCCTCAGGCC
AACCTGACCGAGCTGGACATCTACAGCCGCAGGCTGAGCCAGGAGACCGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCTCTGCAGGACAAGGGCAACAGCACCCACAGCCGCAAC
AACAGCTACGCCGTGATCATCACCAGCACCAGCAGCTACTACGTGTTCTACATCTACGTG
GGAGTGGCCGACACCCTGCTGGCCATGGGCTTCTTCCGCGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGTGGCATCCTGAACCGCTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCCGTGATCGTGGCCTTCATCATGCTGCGCGCCTACTTCCTGCAGACCAGCCAGCAGCTG
AAGCAGCTGGAGAGCGAGGGCCGCAGCCCCATCTTCACCCACCTGGTGACCAGCCTGAAG
GGCCTGTGGACCCTGCGCGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGCGCATCGAGATGATCTTCGTGATCTTCTTCATCGCCGTGACCTTCATCAGCATCCTG
ACCACCGGCGAGGGCGAGGGACGCGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCCACCGAGGGCAAGCCTACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCTCAATCAGCCCCGGCCAGCGCGTG
GGCCTGCTGGGCCGCACCGGCTCAGGCAAGAGCACCCTGCTGAGCGCCTTCCTGCGCCTG
CTGAACACCGAGGGCGAGATCCAGATCGACGGCGTGAGCTGGGACAGCATCACCCTGCAG
CAGTGGCGCAAGGCCTTCGGCGTGATCCCCCAGAAGGTGTTCATCTTCTCTGGCACCTTC
CGCAAGAACCTGGACCCCTACGAGCAGTGGAGCGACCAGGAGATCTGGAAGGTGGCCGAC
GAGGTGGGCCTGCGCAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCCGCAGCGTG
CTGAGCAAGGCCAAGATCCTGCTGCTGGACGAGCCCAGCGCCCACCTGGACCCCGTGACC
TACCAGATCATCCGCCGCACCCTGAAGCAGGCCTTCGCCGACTGCACCGTGATCCTGTGC
GAGCACCGCATCGAGGCCATGCTGGAGTGCCAGCAGTTCCTGGTGATCGAGGAGAACAAG
GTGCGCCAGTACGACTCCATCCAGAAGCTGCTGAACGAGCGCAGCCTGTTCCGCCAGGCC
ATCAGCCCCAGCGACCGCGTGAAGCTTTTCCCCCACCGCAACAGCAGCAAGTGCAAGTCT
AAGCCCCAGATCGCCGCCCTGAAGGAGGAGACCGAGGAGGAGGTGCAGGACACCCGCCTG
TAG
(pARM2090)
SEQ ID NO: 17
ATGCAGAGGTCGCCTCTGGAGAAGGCCAGCGTGGTGTCCAAGCTGTTCTTCAGCTGGACC
AGACCCATCCTGAGGAAGGGCTACCGCCAGCGCCTGGAGCTGAGCGACATCTACCAGATC
CCCAGCGTGGACAGCGCTGACAACCTGAGCGAGAAGCTGGAGAGAGAGTGGGACCGCGAG
CTGGCCAGCAAGAAGAACCCTAAGCTGATCAACGCCCTGCGGCGCTGCTTCTTCTGGCGC
TTCATGTTCTACGGCATCTTCCTGTACCTGGGCGAGGTGACCAAGGCCGTGCAGCCCCTG
CTGCTGGGCCGCATCATCGCCAGCTACGACCCCGACAACAAGGAGGAGCGCTCTATCGCC
ATCTACCTGGGCATCGGCCTGTGCCTGCTGTTCATCGTGCGCACCCTGCTGCTGCACCCC
GCCATCTTCGGCCTGCACCACATCGGCATGCAGATGCGCATCGCCATGTTCAGCCTGATC
TACAAGAAGACCCTGAAGCTGAGCAGCCGCGTGCTGGACAAGATCAGTATCGGCCAGCTG
GTGAGCCTGCTGTCCAACAACCTGAACAAGTTCGACGAGGGCCTGGCCCTGGCCCACTTC
GTGTGGATCGCCCCTCTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACCGCGACCAGCGCGCCGGCAAGATCAGCGAGAGACTGGTG
ATCACCAGCGAGATGATCGAGAACATCCAGTCTGTGAAGGCCTACTGCTGGGAGGAGGCC
ATGGAGAAGATGATCGAGAACCTGCGCCAGACCGAGCTGAAGCTGACCCGGAAGGCCGCC
TACGTGCGCTACTTCAACAGCAGCGCCTTCTTCTTCTCAGGCTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGCCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGCTTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGCGCGGCCAGCTGCTGGCCGTGGCCGGCAGCACC
GGCGCCGGCAAGACCAGCCTGCTGATGGTGATCATGGGCGAGCTGGAGCCCAGCGAGGGC
AAGATCAAGCACAGCGGACGCATCAGCTTCTGCAGCCAGTTCTCCTGGATCATGCCTGGC
ACCATCAAGGAGAACATCATCTTCGGCGTGAGCTACGACGAGTACCGCTACCGCAGCGTG
ATCAAGGCCTGCCAGCTGGAGGAGGACATCAGCAAGTTCGCCGAGAAGGACAACATCGTG
CTGGGAGAGGGCGGCATCACCCTGAGCGGCGGCCAGAGGGCCCGCATCAGCCTGGCAAGA
GCAGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCCGC
ATCCTGGTGACCAGCAAGATGGAGCACCTGAAGAAGGCTGACAAGATCCTGATCCTGCAC
GAGGGCAGCAGCTACTTCTACGGCACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACCGCTTCAGCCTGGAGGGCGACGCCCCCGTGTCCTGGACC
GAGACCAAGAAGCAGAGCTTCAAGCAGACCGGCGAGTTCGGCGAGAAGCGCAAGAACTCT
ATCCTGAACCCCATCAACAGCATCCGCAAGTTCAGCATCGTGCAGAAGACCCCACTGCAG
ATGAACGGCATCGAGGAGGACAGCGACGAGCCCCTGGAGCGCCGCCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCCGCAAAGTGAGCCTGGCCCCACAGGCC
AACCTGACCGAGCTGGACATCTACAGCCGCCGCCTGTCTCAGGAGACCGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCCGCAAC
AACAGCTACGCCGTGATCATCACCAGCACCAGCAGCTACTACGTGTTCTACATCTACGTG
GGCGTGGCCGACACCCTGCTGGCCATGGGCTTCTTCAGAGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGCATCCTGAACCGCTTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGTCTGAGGGCAGGAGCCCCATCTTCACCCACCTGGTGACCAGCCTGAAG
GGCCTGTGGACCCTGAGGGCCTTCGGCCGGCAGCCTTACTTCGAGACCCTGTTCCACAAG
GCCCTGAACCTGCACACCGCCAACTGGTTCCTGTACCTGAGCACCCTGCGCTGGTTCCAG
ATGCGCATCGAGATGATCTTCGTGATCTTCTTCATCGCCGTGACCTTCATCAGCATCCTG
ACCACCGGCGAGGGAGAGGGCAGAGTGGGCATCATCCTGACCCTGGCCATGAACATCATG
AGCACCCTGCAGTGGGCTGTGAACAGCAGCATCGACGTGGACAGCCTGATGCGCAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCTCAATCAGCCCCGGCCAGCGCGTG
GGCCTGCTGGGACGCACCGGCAGCGGCAAGAGCACCCTGCTGAGCGCCTTCCTGAGACTG
CTGAACACCGAGGGCGAGATCCAGATCGACGGCGTGAGCTGGGACTCAATCACCCTGCAG
CAGTGGAGGAAGGCCTTCGGCGTGATCCCCCAGAAGGTGTTCATCTTCAGCGGCACCTTC
CGCAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCCGCTCTGTG
CTGAGCAAGGCCAAGATCCTGCTGCTGGACGAGCCCAGTGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGCCAGTACGACAGCATCCAGAAGCTGCTGAACGAGAGGAGCCTGTTCCGCCAGGCC
ATCAGCCCCAGCGACAGGGTGAAGCTTTTCCCCCACCGCAACAGCAGCAAGTGCAAGAGC
AAGCCCCAGATCGCCGCCCTGAAGGAGGAGACCGAGGAGGAGGTGCAGGACACCAGGCTG
TAG
(pARM2091)
SEQ ID NO: 18
ATGCAGCGCAGCCCCCTGGAGAAGGCCAGCGTGGTGTCCAAGCTGTTCTTCAGCTGGACC
CGCCCCATCCTGAGGAAGGGCTACCGCCAGCGCCTGGAGCTGAGCGACATCTACCAGATC
CCTAGCGTGGACAGCGCCGACAACCTGAGCGAGAAGCTGGAGCGCGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGCCGCATCATCGCCTCCTACGACCCCGACAACAAGGAGGAGCGCAGCATCGCC
ATCTACCTGGGCATCGGCCTGTGCCTGCTGTTCATCGTGCGCACCCTGCTGCTGCACCCC
GCCATCTTCGGCCTGCACCACATCGGCATGCAGATGCGCATCGCCATGTTCAGCCTGATC
TACAAGAAGACCCTGAAGCTGAGCAGCCGCGTGCTGGACAAGATCAGCATCGGCCAGCTG
GTGAGCCTGCTGTCCAACAACCTGAACAAGTTCGACGAGGGACTGGCCCTGGCCCACTTC
GTGTGGATCGCCCCTCTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACCGCGACCAGCGCGCCGGCAAGATCAGCGAGCGCCTGGTG
ATCACCAGCGAGATGATCGAGAACATCCAGAGCGTGAAGGCCTACTGCTGGGAGGAGGCC
ATGGAGAAGATGATCGAGAACCTGAGACAGACCGAGCTGAAGCTGACCCGCAAGGCCGCC
TACGTGAGATACTTCAACAGCAGCGCCTTCTTCTTCAGCGGCTTCTTCGTGGTGTTCCTG
TCTGTGCTGCCCTACGCCCTGATCAAGGGCATCATCCTGCGCAAGATCTTCACCACCATC
TCATTCTGCATCGTGCTGCGCATGGCCGTGACCCGGCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCTGTG
CTGAAGGACATCAACTTCAAGATCGAGCGCGGCCAGCTGCTGGCCGTGGCCGGCAGCACC
GGCGCCGGCAAGACCAGCCTGCTGATGGTGATCATGGGAGAGCTGGAGCCTAGCGAGGGC
AAGATCAAGCACAGTGGACGCATCAGCTTCTGCAGCCAGTTCTCCTGGATCATGCCTGGC
ACCATCAAGGAGAACATCATCTTCGGCGTGTCCTACGACGAGTACCGCTACAGAAGCGTG
ATCAAGGCCTGCCAGCTGGAGGAGGACATCAGCAAGTTCGCCGAGAAGGACAACATCGTG
CTGGGAGAGGGCGGCATCACCCTGAGCGGAGGCCAGCGCGCCAGAATCAGCCTGGCCAGA
GCCGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCAGG
ATCCTGGTGACCTCTAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCAGACTTC
AGCAGCAAGCTGATGGGCTGCGACAGCTTCGACCAGTTCAGCGCCGAGCGCCGCAACAGC
ATCCTGACCGAGACCCTGCACCGCTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCCATCAACAGCATCAGGAAGTTCTCCATCGTGCAGAAGACCCCACTGCAG
ATGAACGGCATCGAGGAGGACAGCGACGAGCCCCTGGAGCGCCGCCTGAGCCTGGTGCCA
GACAGCGAGCAGGGCGAGGCCATCCTGCCCCGCATCAGCGTGATCAGCACCGGCCCCACC
CTGCAGGCCCGCAGGAGGCAGAGCGTGCTGAACCTGATGACCCACAGCGTGAACCAGGGC
CAGAACATCCACAGGAAGACCACCGCCTCCACCCGCAAAGTGAGCCTGGCCCCACAGGCC
AACCTGACCGAGCTGGACATCTACAGCCGCCGCCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCCGCAAC
AACAGCTACGCCGTGATCATCACCAGCACCAGCAGCTACTACGTGTTCTACATCTACGTG
GGCGTGGCCGACACCCTGCTGGCCATGGGCTTCTTCCGCGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGTGGCATCCTGAACCGCTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCCGTGATCGTGGCCTTCATCATGCTGAGAGCCTACTTCCTGCAGACCAGCCAGCAGCTG
AAGCAGCTGGAGAGCGAGGGCCGCAGTCCAATCTTCACCCACCTGGTGACCAGCCTGAAG
GGCCTGTGGACCCTGCGCGCCTTCGGCCGCCAGCCCTACTTCGAGACCCTGTTCCACAAG
GCTCTGAACCTGCACACCGCCAACTGGTTCCTGTACCTGAGCACCCTGCGCTGGTTCCAG
ATGCGCATCGAGATGATCTTCGTGATCTTCTTCATCGCCGTGACCTTCATCAGCATCCTG
ACCACCGGCGAGGGAGAGGGCAGAGTGGGCATCATCCTGACCCTGGCCATGAACATCATG
AGCACCCTGCAGTGGGCCGTGAACTCCAGCATCGACGTGGACAGCCTGATGAGGTCTGTG
AGCCGCGTGTTCAAGTTCATCGACATGCCAACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCTCAATCAGCCCCGGCCAGCGCGTG
GGCCTGCTGGGACGCACCGGCAGCGGCAAGAGCACCCTGCTGAGCGCCTTCCTGAGACTG
CTGAACACCGAGGGCGAGATCCAGATCGACGGCGTGTCTTGGGACTCAATCACCCTGCAG
CAGTGGCGCAAGGCCTTCGGCGTGATCCCCCAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGCGCAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCCGTGACC
TACCAGATCATCCGCAGAACCCTGAAGCAGGCCTTCGCCGACTGCACCGTGATCCTGTGC
GAGCACCGCATCGAGGCCATGCTGGAGTGCCAGCAGTTCCTGGTGATCGAGGAGAACAAG
GTGCGCCAGTACGACAGCATCCAGAAGCTGCTGAACGAGCGCAGCCTGTTCCGCCAGGCC
ATCAGCCCCAGCGACCGCGTGAAGCTTTTCCCCCACCGCAACAGCAGCAAGTGCAAGTCT
AAGCCCCAGATCGCCGCCCTGAAGGAGGAGACCGAGGAGGAGGTGCAGGACACCAGGCTG
TAG
(pARM2092)
SEQ ID NO: 19
ATGCAGCGCAGCCCCCTGGAGAAGGCCAGCGTGGTGAGCAAGCTGTTCTTCAGCTGGACC
CGCCCAATCCTGCGCAAGGGCTACCGCCAGCGCCTGGAGCTGAGCGACATCTACCAGATC
CCCAGCGTGGACAGCGCCGACAACCTGAGCGAGAAGCTGGAGAGAGAGTGGGACCGCGAG
CTGGCCAGCAAGAAGAACCCTAAGCTGATCAACGCCCTGCGGCGCTGCTTCTTCTGGCGC
TTCATGTTCTACGGCATCTTCCTGTACCTGGGCGAGGTGACCAAGGCCGTGCAGCCCCTG
CTGCTGGGCCGCATCATCGCCTCCTACGACCCCGACAACAAGGAGGAGCGCAGCATCGCC
ATCTACCTGGGCATCGGCCTGTGCCTGCTGTTCATCGTGCGCACCCTGCTGCTGCACCCC
GCCATCTTCGGCCTGCACCACATCGGCATGCAGATGCGCATCGCCATGTTCAGCCTGATC
TACAAGAAGACCCTGAAGCTGAGCAGCCGCGTGCTGGACAAGATCAGCATCGGCCAGCTG
GTGAGCCTGCTGTCCAACAACCTGAACAAGTTCGACGAGGGCCTGGCCCTGGCCCACTTC
GTGTGGATCGCCCCACTGCAGGTGGCCCTGCTGATGGGGCTGATCTGGGAGCTGCTGCAG
GCCTCTGCCTTCTGCGGCCTGGGCTTCCTGATCGTGCTGGCCCTGTTCCAGGCCGGCCTG
GGCAGAATGATGATGAAGTACAGAGACCAGCGCGCCGGCAAGATCAGCGAGCGCCTGGTG
ATCACCTCAGAGATGATCGAGAACATCCAGTCTGTGAAGGCCTACTGCTGGGAGGAGGCC
ATGGAGAAGATGATCGAGAACCTGCGCCAGACCGAGCTGAAGCTGACCCGCAAGGCCGCC
TACGTGCGCTACTTCAACAGCAGCGCCTTCTTCTTCTCAGGCTTCTTCGTGGTGTTCCTG
AGCGTGCTGCCCTACGCCCTGATCAAGGGCATCATCCTGCGCAAGATCTTCACCACCATC
AGCTTCTGCATCGTGCTGCGCATGGCCGTGACCCGCCAGTTCCCCTGGGCCGTGCAGACC
TGGTACGACTCTCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGCTTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCCGTG
CTGAAGGACATCAACTTCAAGATCGAGCGCGGCCAGCTGCTGGCCGTGGCCGGCAGCACC
GGCGCCGGCAAGACCAGCCTGCTGATGGTGATCATGGGCGAGCTGGAGCCTAGCGAGGGC
AAGATCAAGCACAGCGGCAGAATCTCATTCTGCTCTCAGTTCAGCTGGATCATGCCTGGC
ACCATCAAGGAGAACATCATCTTCGGCGTGTCCTACGACGAGTACCGCTACCGCAGCGTG
ATCAAGGCCTGCCAGCTGGAGGAGGACATCAGCAAGTTCGCCGAGAAGGACAACATCGTG
CTGGGAGAGGGCGGCATCACCCTGAGCGGCGGCCAGCGCGCCCGCATCAGCCTGGCCCGC
GCCGTGTACAAGGACGCCGACCTGTACCTGCTGGACTCTCCTTTCGGCTACCTGGACGTG
CTGACCGAGAAGGAGATCTTCGAGAGCTGCGTGTGCAAGCTGATGGCCAACAAGACCCGC
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCAGACTTC
AGCAGCAAGCTGATGGGCTGCGACAGCTTCGACCAGTTCAGCGCCGAGCGCCGCAACAGC
ATCCTGACCGAGACCCTGCACCGCTTCAGCCTGGAGGGCGACGCCCCCGTGAGCTGGACC
GAGACCAAGAAGCAGTCTTTCAAGCAGACCGGCGAGTTCGGCGAGAAGAGGAAGAACAGC
ATCCTGAACCCAATCAACAGCATCCGCAAGTTCAGCATCGTGCAGAAGACCCCACTGCAG
ATGAACGGCATCGAGGAGGACAGCGACGAGCCTCTGGAGCGCCGCCTGTCCCTGGTGCCC
GACAGCGAGCAGGGCGAGGCCATCCTGCCCCGCATCAGCGTGATCAGCACCGGCCCCACC
CTGCAGGCCCGCCGCCGCCAGAGCGTGCTGAACCTGATGACCCACAGCGTGAACCAGGGC
CAGAACATCCACCGCAAGACCACCGCCAGCACCCGCAAAGTGAGCCTGGCCCCACAGGCC
AACCTGACCGAGCTGGACATCTACAGCAGACGCCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCCGCAAC
AACAGCTACGCCGTGATCATCACCAGCACCAGCAGCTACTACGTGTTCTACATCTACGTG
GGCGTGGCCGACACCCTGCTGGCCATGGGCTTCTTCCGCGGCCTGCCCCTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGTGGCATCCTGAACCGCTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCAGTGATCGTGGCCTTCATCATGCTGCGCGCCTACTTCCTGCAGACCAGCCAGCAGCTG
AAGCAGCTGGAGAGCGAGGGCCGCAGCCCCATCTTCACCCACCTGGTGACCAGCCTGAAG
GGCCTGTGGACCCTGCGCGCCTTCGGCCGCCAGCCTTACTTCGAGACCCTGTTCCACAAG
GCTCTGAACCTGCACACCGCCAACTGGTTCCTGTACCTGAGCACCCTGCGCTGGTTCCAG
ATGCGCATCGAGATGATCTTCGTGATCTTCTTCATCGCCGTGACCTTCATCAGCATCCTG
ACCACCGGCGAGGGCGAGGGCCGCGTGGGCATCATCCTGACCCTGGCCATGAACATCATG
AGCACCCTGCAGTGGGCCGTGAACAGCAGCATCGACGTGGACAGCCTGATGCGCAGCGTG
AGCCGCGTGTTCAAGTTCATCGACATGCCCACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCTCCTTCAGCATCAGCCCCGGCCAGCGCGTG
GGCCTGCTGGGCCGCACCGGCAGCGGCAAGAGCACCCTGCTGAGCGCCTTCCTGCGCCTG
CTGAACACCGAGGGCGAGATCCAGATCGACGGCGTGTCTTGGGACAGCATCACCCTGCAG
CAGTGGCGCAAGGCCTTCGGCGTGATCCCCCAGAAGGTGTTCATCTTCAGCGGCACCTTC
CGCAAGAACCTGGACCCCTACGAGCAGTGGAGCGACCAGGAGATCTGGAAGGTGGCCGAC
GAGGTGGGCCTGCGCAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCCGCAGCGTG
CTGAGCAAGGCCAAGATCCTGCTGCTGGACGAGCCCAGCGCCCACCTGGACCCCGTGACC
TACCAGATCATCCGCCGCACCCTGAAGCAGGCCTTCGCCGACTGCACCGTGATCCTGTGC
GAGCACCGCATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGCCAGGCC
ATCAGCCCCAGCGACCGCGTGAAGCTTTTCCCCCACCGCAACAGCAGCAAGTGCAAGAGC
AAGCCCCAGATCGCCGCCCTGAAGGAGGAGACCGAGGAGGAGGTGCAGGACACCAGGCTG
TAG
(pARM2093)
SEQ ID NO: 20
ATGCAGAGGTCGCCCCTGGAGAAGGCTAGCGTGGTGAGCAAGCTGTTCTTCAGCTGGACC
AGACCAATCCTGAGGAAGGGCTACAGACAGCGCCTGGAGCTGTCAGACATCTACCAGATC
CCTAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCTCTATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGTATCGGACAGCTG
GTGAGCCTGCTGAGCAACAACCTGAACAAGTTCGACGAGGGACTGGCCCTGGCCCACTTC
GTGTGGATCGCCCCTCTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCTCCTGGATCATGCCCGGC
ACCATCAAGGAGAACATCATCTTCGGTGTGAGCTACGACGAGTACAGATACAGAAGCGTG
ATCAAGGCCTGCCAGCTGGAGGAGGACATCTCCAAGTTCGCAGAGAAGGACAACATCGTG
CTGGGAGAGGGTGGCATCACCCTGAGCGGAGGCCAGAGGGCCAGAATCTCTCTGGCAAGA
GCAGTGTACAAGGACGCCGACCTGTACCTGCTGGACTCTCCCTTCGGATACCTGGACGTG
CTGACCGAGAAGGAGATCTTCGAGAGCTGCGTGTGCAAGCTGATGGCCAACAAGACCAGG
ATCCTGGTGACCTCTAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCTCCATCGTGCAGAAGACCCCACTGCAG
ATGAACGGCATCGAGGAGGACAGCGACGAGCCCCTGGAGAGAAGGCTGAGCCTGGTGCCA
GACTCTGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGTGGGATCCTGAACAGATTCTCCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGTCTGAGGGCAGGAGTCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
GCTGAACACCGAGGGCGAGATCCAGATCGACGGCGTGAGCTGGACTCAATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGATCTGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2094)
SEQ ID NO: 21
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGTCAGACATCTACCAGATC
CCCAGCGTGGACTCTGCCGACAACCTGTCTGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCTGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGTGTGTCCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCTGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGTCCTGGACC
GAGACCAAGAAGCAGTCTTTCAAGCAGACCGGAGAGTTCGGCGAGAAGAGGAAGAACAGC
ATCCTGAACCCAATCAACTCTATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCTCGCATCAGCGTGATCAGCACCGGCCCCACC
CTGCAGGCCAGGAGGAGGCAGTCTGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
GCTGATCACCGTGAGCAAGATCCTGCACCACAAGATGCTGCACAGCGTCTGCAGGCCCCC
ATGAGCACCCTGAACACCCTGAAGGCCGGTGGGATCCTGAACAGATTCTCCAAGGACATC
GCCATCCTGGACGACCTGCTGCCTCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACTCCAGCATCGACGTGGACAGCCTGATGAGGAGCGTG
AGCAGGGTGTTCAAGTTCATCGACATGCCAACCGAGGGCAAGCCTACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCTGGCAAGCTGGACTTCGTGCTGGTG
GACGGGGGCTGCGTGCTGAGCCACGGCCACAAGCAGCTGATGTGCCTGGCCAGATCTGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2095)
SEQ ID NO: 22
ATGCAGAGGTCGCCCCTGGAGAAGGCTAGCGTGGTGTCCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCTCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGCATCGGACAGCTG
GTGAGTCTGCTGAGCAACAACCTGAACAAGTTCGACGAGGGACTGGCCCTGGCCCACTTC
GTGTGGATCGCTCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCTCAGAGATGATCGAGAACATCCAGTCTGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCTCTAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGATCCACC
GGAGCCGGCAAGACCAGCCTGCTGATGGTGATCATGGGAGAGCTGGAGCCCTCAGAGGGC
AAGATCAAGCACAGTGGAAGAATCTCATTCTGCAGCCAGTTCTCCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGTCTTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCTCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCTCAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCT
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGTCCAATCTTCACCCACCTGGTGACCAGCCTGAAG
GGACTGTGGACCCTGAGGGCCTTCGGCCGGCAGCCTTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCTCCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCTCAGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACTCCATCCAGAAGCTGCTGAACGAGAGGAGCCTGTTCCGGCAGGCC
ATCAGCCCCTCCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2096)
SEQ ID NO: 23
ATGCAGAGGTCGCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCTAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCTCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGTATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCTCAGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCTCTAACGGCGACGACAGCCTGTTCTTCAGCAACTTCAGCCTGCTGGGCACCCCTGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCTCAGAGGGC
AAGATCAAGCACAGCGGAAGAATCAGCTTCTGCAGCCAGTTCAGCTGGATCATGCCCGGC
ACCATCAAGGAGAACATCATCTTCGGTGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCTCTCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGTCCTGGACC
GAGACCAAGAAGCAGAGCTTCAAGCAGACCGGAGAGTTCGGCGAGAAGAGGAAGAACAGC
ATCCTGAACCCAATCAACTCTATCAGGAAGTTCAGCATCGTGCAGAAGACCCCACTGCAG
ATGAACGGCATCGAGGAGGACAGCGACGAGCCCCTGGAGAGAAGGCTGTCCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCTCTCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCT
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCTCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCTCCATCCTG
ACCACCGGCGAGGGAGAGGGAAGAGTGGGCATCATCCTGACCCTGGCCATGAACATCATG
AGCACCCTGCAGTGGGCTGTGAACAGCAGCATCGACGTGGACAGCCTGATGAGGAGCGTG
AGCAGGGTGTTCAAGTTCATCGACATGCCAACCGAGGGCAAGCCTACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCTCAATCAGCCCTGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCTGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGTCT
AAGCCCCAGATCGCCGCCCTGAAGGAGGAGACCGAGGAGGAGGTGCAGGACACCAGGCTG
TAG
(pARM2097)
SEQ ID NO: 24
ATGCAGAGGAGCCCCCTGGAGAAGGCCAGCGTGGTGAGCAAGCTGTTCTTCAGCTGGACC
AGACCAATCCTGCGCAAGGGCTACCGCCAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGCAGGTGCTTCTTCTGGAGA
TTCATGTTCTACGGAATCTTCCTGTACCTGGGCGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGCGCACCCTGCTGCTGCACCCA
GCCATCTTCGGCCTGCACCACATCGGAATGCAGATGCGCATCGCCATGTTCAGCCTGATC
TACAAGAAGACCCTGAAGCTGAGCAGCCGCGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGCCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGCGCGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGCGCTACTTCAACAGCAGCGCCTTCTTCTTCAGCGGGTTCTTCGTGGTGTTCCTG
AGCGTGCTGCCCTACGCCCTGATCAAGGGCATCATCCTGCGCAAGATCTTCACCACCATC
AGCTTCTGCATCGTGCTGCGCATGGCCGTGACCCGCCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCCGTG
CTGAAGGACATCAACTTCAAGATCGAGCGCGGACAGCTGCTGGCCGTGGCCGGAAGCACC
GGAGCCGGCAAGACCAGCCTGCTGATGGTGATCATGGGCGAGCTGGAGCCCAGCGAGGGC
AAGATCAAGCACAGCGGACGCATCAGCTTCTGCAGCCAGTTCAGCTGGATCATGCCCGGC
ACCATCAAGGAGAACATCATCTTCGGCGTGAGCTACGACGAGTACAGATACCGCAGCGTG
ATCAAGGCCTGCCAGCTGGAGGAGGACATCAGCAAGTTCGCAGAGAAGGACAACATCGTG
CTGGGCGAGGGCGGCATCACCCTGAGCGGCGGCCAGAGGGCCAGAATCAGCCTGGCACGC
GCAGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCCGC
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGAGAAGAAACAGC
ATCCTGACCGAGACCCTGCACCGCTTCAGCCTGGAGGGCGACGCCCCCGTGAGCTGGACC
GAGACCAAGAAGCAGAGCTTCAAGCAGACCGGCGAGTTCGGCGAGAAGCGCAAGAACAGC
ATCCTGAACCCCATCAACAGCATCAGGAAGTTCAGCATCGTGCAGAAGACCCCACTGCAG
ATGAACGGCATCGAGGAGGACAGCGACGAGCCCCTGGAGCGCAGGCTGAGCCTGGTGCCA
GACAGCGAGCAGGGCGAGGCCATCCTGCCCCGCATCAGCGTGATCAGCACCGGCCCCACC
CTGCAGGCCAGGCGCCGCCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGACGCCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCCGCAGCCCCATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGCGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGCGCGTG
GGCCTGCTGGGACGCACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCCCAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCCGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACCGCGTGAAGCTTTTCCCCCACCGCAACAGCAGCAAGTGCAAGAGC
AAGCCCCAGATCGCCGCCCTGAAGGAGGAGACCGAGGAGGAGGTGCAGGACACCAGGCTG
TAG
(pARM2098)
SEQ ID NO: 25
ATGCAGCGCAGCCCCCTGGAGAAGGCCAGCGTGGTGAGCAAGCTGTTCTTCAGCTGGACC
CGCCCCATCCTGCGCAAGGGCTACCGCCAGCGCCTGGAGCTGAGCGACATCTACCAGATC
CCCAGCGTGGACAGCGCCGACAACCTGAGCGAGAAGCTGGAGCGCGAGTGGGACAGAGAG
CTGGCCAGCAAGAAGAACCCCAAGCTGATCAACGCCCTGCGCAGGTGCTTCTTCTGGCGC
TTCATGTTCTACGGAATCTTCCTGTACCTGGGCGAGGTGACCAAGGCCGTGCAGCCCCTG
CTGCTGGGCCGCATCATCGCCAGCTACGACCCCGACAACAAGGAGGAGCGCAGCATCGCC
ATCTACCTGGGCATCGGCCTGTGCCTGCTGTTCATCGTGCGCACCCTGCTGCTGCACCCC
GCCATCTTCGGCCTGCACCACATCGGAATGCAGATGCGCATCGCCATGTTCAGCCTGATC
TACAAGAAGACCCTGAAGCTGAGCAGCCGCGTGCTGGACAAGATCAGCATCGGCCAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGCCTGATCTGGGAGCTGCTGCAG
GCCAGCGCCTTCTGCGGCCTGGGCTTCCTGATCGTGCTGGCCCTGTTCCAGGCCGGCCTG
GGCCGCATGATGATGAAGTACCGCGACCAGCGCGCCGGCAAGATCAGCGAGAGACTGGTG
ATCACCAGCGAGATGATCGAGAACATCCAGAGCGTGAAGGCCTACTGCTGGGAGGAGGCC
ATGGAGAAGATGATCGAGAACCTGAGACAGACCGAGCTGAAGCTGACCCGGAAGGCCGCC
TACGTGCGCTACTTCAACAGCAGCGCCTTCTTCTTCAGCGGCTTCTTCGTGGTGTTCCTG
AGCGTGCTGCCCTACGCCCTGATCAAGGGCATCATCCTGCGCAAGATCTTCACCACCATC
AGCTTCTGCATCGTGCTGCGCATGGCCGTGACCCGCCAGTTCCCCTGGGCCGTGCAGACC
TGGTACGACAGCCTGGGCGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCCGTG
CTGAAGGACATCAACTTCAAGATCGAGCGCGGCCAGCTGCTGGCCGTGGCCGGAAGCACC
GGAGCCGGCAAGACCAGCCTGCTGATGGTGATCATGGGCGAGCTGGAGCCCAGCGAGGGC
AAGATCAAGCACAGCGGCCGCATCAGCTTCTGCAGCCAGTTCAGCTGGATCATGCCCGGC
ACCATCAAGGAGAACATCATCTTCGGCGTGAGCTACGACGAGTACCGCTACCGCAGCGTG
ATCAAGGCCTGCCAGCTGGAGGAGGACATCAGCAAGTTCGCCGAGAAGGACAACATCGTG
CTGGGCGAGGGCGGCATCACCCTGAGCGGCGGCCAGCGCGCCCGCATCAGCCTGGCCCGC
GCCGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCCGC
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGAGAAGAAACAGC
ATCCTGACCGAGACCCTGCACCGCTTCAGCCTGGAGGGCGACGCCCCCGTGAGCTGGACC
GAGACCAAGAAGCAGAGCTTCAAGCAGACCGGCGAGTTCGGCGAGAAGCGCAAGAACAGC
ATCCTGAACCCCATCAACAGCATCCGCAAGTTCAGCATCGTGCAGAAGACCCCACTGCAG
ATGAACGGCATCGAGGAGGACAGCGACGAGCCCCTGGAGCGCCGCCTGAGCCTGGTGCCC
GACAGCGAGCAGGGCGAGGCCATCCTGCCCCGCATCAGCGTGATCAGCACCGGCCCCACC
CTGCAGGCCCGCCGCCGCCAGAGCGTGCTGAACCTGATGACCCACAGCGTGAACCAGGGC
CAGAACATCCACCGCAAGACCACCGCCAGCACCCGCAAAGTGAGCCTGGCCCCACAGGCC
AACCTGACCGAGCTGGACATCTACAGCAGACGCCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGCGCTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGCGTGGCCGACACCCTGCTGGCCATGGGCTTCTTCCGCGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACCGCTTCAGCAAGGACATC
GCCATCCTGGACGACCTGCTGCCCCTGACCATCTTCGACTTCATCCAGCTGCTGCTGATC
GTGATCGGCGCCATCGCCGTGGTGGCCGTGCTGCAGCCCTACATCTTCGTGGCCACCGTG
CCCGTGATCGTGGCCTTCATCATGCTGAGAGCCTACTTCCTGCAGACCAGCCAGCAGCTG
AAGCAGCTGGAGAGCGAGGGCCGCAGCCCCATCTTCACCCACCTGGTGACCAGCCTGAAG
GGCCTGTGGACCCTGAGGGCCTTCGGCCGCCAGCCCTACTTCGAGACCCTGTTCCACAAG
GCCCTGAACCTGCACACCGCCAACTGGTTCCTGTACCTGAGCACCCTGCGCTGGTTCCAG
ATGCGCATCGAGATGATCTTCGTGATCTTCTTCATCGCCGTGACCTTCATCAGCATCCTG
ACCACCGGCGAGGGCGAGGGACGCGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCCACCGAGGGCAAGCCCACCAAGAGCACCAAG
CCCTACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGCGCGTG
GGCCTGCTGGGCCGCACCGGCAGCGGCAAGAGCACCCTGCTGAGCGCCTTCCTGCGCCTG
CTGAACACCGAGGGCGAGATCCAGATCGACGGCGTGAGCTGGGACAGCATCACCCTGCAG
CAGTGGCGCAAGGCCTTCGGCGTGATCCCCCAGAAGGTGTTCATCTTCAGCGGCACCTTC
CGCAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCCGCAGCGTG
CTGAGCAAGGCCAAGATCCTGCTGCTGGACGAGCCCAGCGCCCACCTGGACCCCGTGACC
TACCAGATCATCAGACGCACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGCCAGTACGACAGCATCCAGAAGCTGCTGAACGAGCGCAGCCTGTTCCGCCAGGCC
ATCAGCCCCAGCGACCGCGTGAAGCTTTTCCCCCACCGCAACAGCAGCAAGTGCAAGAGC
AAGCCCCAGATCGCCGCCCTGAAGGAGGAGACCGAGGAGGAGGTGCAGGACACCCGCCTG
TAG
(pARM2099)
SEQ ID NO: 26
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCTCCATCCTG
ACCACCGGCGAGGGAGAGGGAAGAGTGGGCATCATCCTGACCCTGGCCATGAACATCATG
AGCACCCTGCAGTGGGCTGTGAACTCCAGCATCGACGTGGACAGCCTGATGAGGTCTGTG
AGCAGGGTGTTCAAGTTCATCGACATGCCAACCGAGGGCAAGCCTACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCTCCTTCTCAATCAGCCCTGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGTCTTGGGACTCAATCACCCTGCAG
CAGTGGAGGAAGGCCTTCGGCGTGATCCCACAGAAGGTGTTCATCTTCTCTGGAACCTTC
AGAAAGAACCTGGACCCCTACGAGCAGTGGAGCGACCAGGAGATCTGGAAGGTGGCCGAC
GAGGTGGGCCTGAGATCTGTGATCGAGCAGTTCCCTGGCAAGCTGGACTTCGTGCTGGTG
GACGGGGGCTGCGTGCTGAGCCACGGCCACAAGCAGCTGATGTGCCTGGCCAGATCTGTG
CTGAGCAAGGCCAAGATCCTGCTGCTGGACGAGCCCAGTGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACTCCATCCAGAAGCTGCTGAACGAGAGGAGCCTGTTCCGGCAGGCC
ATCAGCCCCTCCGACAGGGTGAAGCTGTTCCCCCACCGGAACAGCAGCAAGTGCAAGTCT
AAGCCCCAGATCGCCGCCCTGAAGGAGGAGACCGAGGAGGAGGTGCAGGACACCAGGCTG
TAG
(pARM2101)
SEQ ID NO: 27
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGATCCACC
GGAGCCGGCAAGACCTCACTGCTGATGGTGATCATGGGAGAGCTGGAGCCTTCAGAGGGC
AAGATCAAGCACAGTGGAAGAATCAGCTTCTGCAGCCAGTTCTCCTGGATCATGCCCGGC
ACCATCAAGGAGAACATCATCTTCGGTGTGTCCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGTGGCATCACCCTGAGCGGAGGCCAGAGGGCCAGAATCTCTCTGGCAAGA
GCAGTGTACAAGGACGCCGACCTGTACCTGCTGGACTCTCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGTCCTGGACC
GAGACCAAGAAGCAGTCTTTCAAGCAGACCGGAGAGTTCGGCGAGAAGAGGAAGAACTCT
ATCCTGAACCCAATCAACTCTATCAGGAAGTTCTCCATCGTGCAGAAGACCCCACTGCAG
ATGAACGGCATCGAGGAGGACTCTGACGAGCCCCTGGAGAGAAGGCTGTCCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2102)
SEQ ID NO: 28
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCTCTAACGGCGACGACAGCCTGTTCTTCAGCAACTTCAGCCTGCTGGGCACCCCTGTG
CTGAAGGACATCAACTTCAAGATCGAGAGAGGACAGCTGCTGGCCGTGGCCGGATCCACC
GGAGCCGGCAAGACCAGCCTGCTGATGGTGATCATGGGAGAGCTGGAGCCTAGCGAGGGC
AAGATCAAGCACAGTGGAAGAATCTCATTCTGCTCTCAGTTCAGCTGGATCATGCCTGGC
ACCATCAAGGAGAACATCATCTTCGGTGTGTCCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGTGGCATCACCCTGAGCGGAGGCCAGAGGGCCAGAATCAGCCTGGCAAGA
GCAGTGTACAAGGACGCCGACCTGTACCTGCTGGACTCTCCTTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCTGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCTGTGAGCTGGACC
GAGACCAAGAAGCAGTCTTTCAAGCAGACCGGAGAGTTCGGCGAGAAGAGGAAGAACTCT
ATCCTGAACCCAATCAACAGCATCAGGAAGTTCTCCATCGTGCAGAAGACCCCACTGCAG
ATGAACGGCATCGAGGAGGACAGCGACGAGCCTCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2103)
SEQ ID NO: 29
ATGCAGAGGAGCCCTCTGGAGAAGGCTAGCGTGGTGTCCAAGCTGTTCTTCAGCTGGACC
AGACCAATCCTGAGGAAGGGCTACAGACAGCGCCTGGAGCTGTCAGACATCTACCAGATC
CCTTCTGTGGACTCTGCTGACAACCTGTCTGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCTCTG
CTGCTGGGAAGAATCATCGCCAGCTACGACCCCGACAACAAGGAGGAGCGCTCTATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGTATCGGACAGCTG
GTGAGTCTGCTGTCCAACAACCTGAACAAGTTCGACGAGGGACTGGCCCTGGCCCACTTC
GTGTGGATCGCTCCTCTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCTCAGGGTTCTTCGTGGTGTTCCTG
TCTGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2104)
SEQ ID NO: 30
ATGCAGAGGTCGCCTCTGGAGAAGGCTAGCGTGGTGTCCAAGCTGTTCTTCAGCTGGACC
AGACCAATCCTGAGGAAGGGCTACAGACAGCGCCTGGAGCTGAGCGACATCTACCAGATC
CCCTCTGTGGACAGCGCTGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCTCCTACGACCCCGACAACAAGGAGGAGCGCTCTATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGTATCGGACAGCTG
GTGAGTCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCTCTGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCTCAGAGATGATCGAGAACATCCAGTCTGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCTCAGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2105)
SEQ ID NO: 31
ATGCAGCGCAGCCCCCTGGAGAAGGCCAGCGTGGTGAGCAAGCTGTTCTTCAGCTGGACC
CGCCCCATCCTGCGCAAGGGCTACCGCCAGCGCCTGGAGCTGAGCGACATCTACCAGATC
CCCAGCGTGGACAGCGCCGACAACCTGAGCGAGAAGCTGGAGCGCGAGTGGGACCGCGAG
CTGGCCAGCAAGAAGAACCCCAAGCTGATCAACGCCCTGCGCCGCTGCTTCTTCTGGAGA
TTCATGTTCTACGGAATCTTCCTATACCTAGGGGAAGTCACCAAAGCAGTACAGCCACTC
CTACTGGGAAGAATCATAGCAAGCTACGACCCGGACAACAAGGAGGAACGCAGTATCGCG
ATATACCTAGGCATAGGCCTATGCCTACTCTTCATAGTGAGGACACTGCTCCTACACCCA
GCCATATTCGGCCTACATCACATAGGAATGCAGATGAGAATAGCAATGTTCAGTCTAATA
TACAAGAAGACACTAAAGCTGTCAAGCCGAGTACTAGACAAAATAAGTATAGGACAACTA
GTAAGTCTCCTAAGCAACAACCTGAACAAATTCGACGAAGGACTAGCACTAGCACATTTC
GTGTGGATCGCACCACTACAAGTGGCACTCCTCATGGGGCTAATCTGGGAGCTACTACAG
GCGAGTGCCTTCTGCGGACTAGGTTTCCTGATAGTCCTAGCCCTATTCCAGGCAGGGCTA
GGGAGAATGATGATGAAGTACAGAGACCAGAGAGCAGGGAAGATCAGTGAAAGACTAGTG
ATAACCTCAGAAATGATAGAAAACATCCAAAGTGTAAAGGCATACTGCTGGGAAGAAGCA
ATGGAGAAAATGATAGAAAACCTAAGACAAACAGAACTGAAACTGACACGGAAGGCAGCC
TACGTGAGATACTTCAACAGCTCAGCCTTCTTCTTCTCAGGGTTCTTCGTGGTGTTCCTG
AGCGTGCTGCCCTACGCCCTGATCAAGGGCATCATCCTGCGCAAGATCTTCACCACCATC
TCATTCTGCATAGTACTGCGCATGGCGGTCACACGGCAATTCCCCTGGGCAGTACAAACA
TGGTACGACAGTCTAGGAGCAATAAACAAAATACAGGACTTCCTACAAAAGCAAGAATAC
AAGACACTAGAATACAACCTAACGACCACCGAGGTGGTGATGGAGAACGTGACCGCCTTC
TGGGAGGAGGGCTTCGGCGAGCTGTTCGAGAAGGCCAAGCAGAACAACAACAACCGCAAG
ACCAGCAACGGCGACGACAGCCTGTTCTTCAGCAACTTCAGCCTGCTGGGCACCCCCGTG
CTGAAGGACATCAACTTCAAGATCGAGCGCGGCCAGCTGCTGGCCGTGGCCGGCAGCACC
GGCGCCGGCAAGACCAGCCTGCTGATGGTGATCATGGGCGAGCTGGAGCCCAGCGAGGGC
AAGATCAAGCACAGCGGCCGCATCAGCTTCTGCAGCCAGTTCAGCTGGATCATGCCCGGC
ACCATCAAGGAGAACATCATCTTCGGCGTGAGCTACGACGAGTACCGCTACCGCAGCGTG
ATCAAGGCCTGCCAGCTGGAGGAGGACATCAGCAAGTTCGCCGAGAAGGACAACATCGTG
CTGGGCGAGGGCGGCATCACCCTGAGCGGCGGCCAGCGCGCCCGCATCAGCCTGGCCCGC
GCCGTGTACAAGGACGCCGACCTGTACCTGCTGGACAGCCCCTTCGGCTACCTGGACGTG
CTGACCGAGAAGGAGATCTTCGAGAGCTGCGTGTGCAAGCTGATGGCCAACAAGACCCGC
ATCCTGGTGACCAGCAAGATGGAGCACCTGAAGAAGGCCGACAAGATCCTGATCCTGCAC
GAGGGCAGCAGCTACTTCTACGGCACCTTCAGCGAGCTGCAGAACCTGCAGCCCGACTTC
AGCAGCAAGCTGATGGGCTGCGACAGCTTCGACCAGTTCAGCGCCGAGCGCCGCAACAGC
ATCCTGACCGAGACCCTGCACCGCTTCAGCCTGGAGGGCGACGCCCCCGTGAGCTGGACC
GAGACCAAGAAGCAGAGCTTCAAGCAGACCGGCGAGTTCGGCGAGAAGCGCAAGAACAGC
ATCCTGAACCCCATCAACAGCATCCGCAAGTTCAGCATCGTGCAGAAGACCCCACTGCAG
ATGAACGGCATCGAGGAGGACAGCGACGAGCCCCTGGAGCGCCGCCTGAGCCTGGTGCCC
GACAGCGAGCAGGGCGAGGCCATCCTGCCCCGCATCAGCGTGATCAGCACCGGCCCCACC
CTGCAGGCCCGCCGCCGCCAGAGCGTGCTGAACCTGATGACCCACAGCGTGAACCAGGGC
CAGAACATCCACCGCAAGACCACCGCCAGCACCCGCAAGGTGAGCCTGGCCCCACAGGCC
AACCTGACCGAGCTGGACATCTACAGCCGCCGCCTGAGCCAGGAGACCGGCCTGGAGATC
AGCGAGGAGATCAACGAGGAGGACCTGAAGGAGTGCTTCTTCGACGACATGGAGAGCATC
CCCGCCGTGACCACCTGGAACACCTACCTGCGCTACATCACCGTGCACAAGAGCCTAATA
TTCGTGCTAATATGGTGCCTAGTAATATTCCTGGCAGAGGTGGCAGCAAGTCTAGTAGTG
CTGTGGCTCCTAGGAAACACACCACTACAAGACAAAGGGAACAGTACACATAGTAGAAAC
AACAGCTACGCAGTGATAATCACCAGCACCAGTTCGTACTACGTGTTCTACATATACGTG
GGAGTAGCCGACACACTACTAGCAATGGGATTCTTCAGAGGTCTACCACTGGTGCATACA
CTAATCACAGTGTCGAAAATACTACACCACAAAATGCTACATAGTGTACTACAAGCACCA
ATGTCAACCCTCAACACGCTAAAAGCAGGTGGGATACTAAACAGATTCAGCAAAGACATA
GCAATACTAGACGACCTACTGCCACTAACCATATTCGACTTCATCCAGCTACTACTAATA
GTGATAGGAGCAATAGCAGTAGTCGCAGTACTACAACCCTACATCTTCGTAGCAACAGTG
CCAGTGATAGTGGCATTCATAATGCTAAGAGCATACTTCCTCCAAACCTCACAGCAACTC
AAACAACTGGAAAGTGAAGGCAGGAGTCCAATATTCACACATCTAGTAACAAGCCTAAAA
GGACTATGGACACTACGAGCCTTCGGACGGCAGCCATACTTCGAAACACTGTTCCACAAA
GCACTGAACCTACATACAGCCAACTGGTTCCTATACCTGTCAACACTGCGCTGGTTCCAA
ATGAGAATAGAAATGATATTCGTCATCTTCTTCATAGCAGTAACCTTCATCAGCATCCTG
ACCACCGGCGAGGGCGAGGGCCGCGTGGGCATAATCCTGACACTAGCCATGAACATCATG
AGTACACTACAGTGGGCAGTAAACAGCAGCATAGACGTGGACAGCCTAATGCGAAGTGTG
AGCCGAGTCTTCAAGTTCATAGACATGCCAACAGAAGGTAAACCAACCAAGTCAACCAAA
CCATACAAGAACGGCCAACTCTCGAAAGTAATGATAATAGAGAACTCACACGTGAAGAAA
GACGACATCTGGCCCTCAGGGGGCCAGATGACCGTGAAGGACCTGACCGCCAAGTACACC
GAGGGCGGCAACGCCATCCTGGAGAACATCAGCTTCAGCATCAGCCCCGGCCAGCGCGTG
GGCCTGCTGGGCCGCACCGGCAGCGGCAAGAGCACCCTGCTGAGCGCCTTCCTGCGCCTG
CTGAACACCGAGGGCGAGATCCAGATCGACGGCGTGAGCTGGGACAGCATCACCCTGCAG
CAGTGGCGCAAGGCCTTCGGCGTGATCCCCCAGAAGGTGTTCATCTTCAGCGGCACCTTC
CGCAAGAACCTGGACCCCTACGAGCAGTGGAGCGACCAGGAGATCTGGAAGGTGGCCGAC
GAGGTGGGCCTGCGCAGCGTGATCGAGCAGTTCCCCGGCAAGCTGGACTTCGTGCTGGTG
GACGGCGGCTGCGTGCTGAGCCACGGCCACAAGCAGCTGATGTGCCTGGCCCGCAGCGTG
CTGAGCAAGGCCAAGATCCTGCTGCTGGACGAGCCCAGCGCCCACCTGGACCCCGTGACC
TACCAGATCATCCGCCGCACCCTGAAGCAGGCCTTCGCCGACTGCACCGTGATCCTGTGC
GAGCACCGCATCGAGGCCATGCTGGAGTGCCAGCAGTTCCTGGTGATCGAGGAGAACAAG
GTGCGCCAGTACGACAGCATCCAGAAGCTGCTGAACGAGCGCAGCCTGTTCCGCCAGGCC
ATCAGCCCCAGCGACCGCGTGAAGCTGTTCCCCCACCGCAACAGCAGCAAGTGCAAGAGC
AAGCCCCAGATCGCCGCCCTGAAGGAGGAGACCGAGGAGGAGGTGCAGGACACCCGCCTG
TAG
(pARM2106)
SEQ ID NO: 32
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2107)
SEQ ID NO: 33
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2108)
SEQ ID NO: 34
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2109)
SEQ ID NO: 35
ATGCAGAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2110)
SEQ ID NO: 36
ATGCCCAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAAGGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2111)
SEQ ID NO: 37
ATGCCCAGGAGCCCCCTGGAGAAGGCTAGCGTGGTGAGCAAGCTGTTCTTCAGCTGGACC
AGACCAATCCTGAGGAAGGGCTACAGACAGCGCCTGGAGCTGAGCGACATCTACCAGATC
CCCAGCGTGGACAGCGCCGACAACCTGAGCGAGAAGCTGGAGAGAGAGTGGGACAGAGAG
CTGGCCAGCAAGAAGAACCCCAAGCTGATCAACGCCCTGCGGAGGTGCTTCTTCTGGAGA
TTCATGTTCTACGGAATCTTCCTGTACCTGGGGGAGGTGACCAAGGCCGTGCAGCCCCTG
CTGCTGGGAAGAATCATCGCCAGCTACGACCCCGACAACAAGGAGGAGCGCAGCATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGAGCAGCAGGGTGCTGGACAAGATCAGCATCGGACAGCTG
GTGAGCCTGCTGAGCAACAACCTGAACAAGTTCGACGAGGGACTGGCCCTGGCCCACTTC
GTGTGGATCGCCCCACTGCAGGTGGCCCTGCTGATGGGGCTGATCTGGGAGCTGCTGCAG
GCCAGCGCCTTCTGCGGCCTGGGCTTCCTGATCGTGCTGGCCCTGTTCCAGGCCGGCCTG
GGCAGAATGATGATGAAGTACAGAGACCAGAGAGCCGGCAAGATCAGCGAGAGACTGGTG
ATCACCAGCGAGATGATCGAGAACATCCAGAGCGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCAGCGGGTTCTTCGTGGTGTTCCTG
AGCGTGCTGCCCTACGCCCTGATCAAGGGCATCATCCTGCGGAAGATCTTCACCACCATC
AGCTTCTGCATCGTGCTGCGCATGGCCGTGACCCGGCAGTTCCCCTGGGCCGTGCAGACC
TGGTACGACAGCCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCAGCAACGGCGACGACAGCCTGTTCTTCAGCAACTTCAGCCTGCTGGGCACCCCCGTG
CTGAAGGACATCAACTTCAAGATCGAGAGAGGACAGCTGCTGGCCGTGGCCGGAAGCACC
GGAGCCGGCAAGACCAGCCTGCTGATGGTGATCATGGGAGAGCTGGAGCCCAGCGAGGGC
AAGATCAAGCACAGCGGAAGAATCAGCTTCTGCAGCCAGTTCAGCTGGATCATGCCCGGC
ACCATCAAGGAGAACATCATCTTCGGCGTGAGCTACGACGAGTACAGATACAGAAGCGTG
ATCAAGGCCTGCCAGCTGGAGGAGGACATCAGCAAGTTCGCAGAGAAGGACAACATCGTG
CTGGGAGAGGGCGGCATCACCCTGAGCGGAGGCCAGAGGGCCAGAATCAGCCTGGCAAGA
GCAGTGTACAAGGACGCCGACCTGTACCTGCTGGACAGCCCCTTCGGATACCTGGACGTG
CTGACCGAGAAGGAGATCTTCGAGAGCTGCGTGTGCAAGCTGATGGCCAACAAGACCAGG
ATCCTGGTGACCAGCAAGATGGAGCACCTGAAGAAGGCCGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACAGCTTCGACCAGTTCAGCGCCGAGAGAAGAAACAGC
ATCCTGACCGAGACCCTGCACAGGTTCAGCCTGGAGGGCGACGCCCCCGTGAGCTGGACC
GAGACCAAGAAGCAGAGCTTCAAGCAGACCGGAGAGTTCGGCGAGAAGAGGAAGAACAGC
ATCCTGAACCCAATCAACAGCATCAGGAAGTTCAGCATCGTGCAGAAGACCCCACTGCAG
ATGAACGGCATCGAGGAGGACAGCGACGAGCCCCTGGAGAGAAGGCTGAGCCTGGTGCCA
GACAGCGAGCAGGGCGAGGCCATCCTGCCCCGCATCAGCGTGATCAGCACCGGCCCCACC
CTGCAGGCCAGGAGGAGGCAGAGCGTGCTGAACCTGATGACCCACAGCGTGAACCAGGGC
CAGAACATCCACAGGAAGACCACCGCCAGCACCAGGAAGGTGAGCCTGGCCCCACAGGCC
AACCTGACCGAGCTGGACATCTACAGCAGAAGGCTGAGCCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCAGCCTGGTGGTG
CTGTGGCTGCTGGGCAACACCCCACTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGCCTGCCACTGGTGCACACC
CTGATCACCGTGAGCAAGATCCTGCACCACAAGATGCTGCACAGCGTGCTGCAGGCCCCC
ATGAGCACCCTGAACACCCTGAAGGCCGGCGGGATCCTGAACAGATTCAGCAAGGACATC
GCCATCCTGGACGACCTGCTGCCCCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGAGCGAGGGCAGGAGCCCAATCTTCACCCACCTGGTGACCAGCCTGAAG
GGACTGTGGACCCTGAGGGCCTTCGGCCGGCAGCCCTACTTCGAGACCCTGTTCCACAAG
GCCCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCAGCATCCTG
ACCACCGGCGAGGGAGAGGGAAGAGTGGGCATCATCCTGACCCTGGCCATGAACATCATG
AGCACCCTGCAGTGGGCCGTGAACAGCAGCATCGACGTGGACAGCCTGATGAGGAGCGTG
AGCAGGGTGTTCAAGTTCATCGACATGCCAACCGAGGGCAAGCCCACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCAGCTTCAGCATCAGCCCCGGCCAGAGGGTG
GGCCTGCTGGGAAGAACCGGCAGCGGCAAGAGCACCCTGCTGAGCGCCTTCCTGAGACTG
CTGAACACCGAGGGCGAGATCCAGATCGACGGCGTGAGCTGGGACAGCATCACCCTGCAG
CAGTGGAGGAAGGCCTTCGGCGTGATCCCACAGAGAGTGTTCATCTTCAGCGGAACCTTC
AGAAAGAACCTGGACCCCTACGAGCAGTGGAGCGACCAGGAGATCTGGAAGGTGGCCGAC
GAGGTGGGCCTGAGAAGCGTGATCGAGCAGTTCCCCGGCAAGCTGGACTTCGTGCTGGTG
GACGGGGGCTGCGTGCTGAGCCACGGCCACAAGCAGCTGATGTGCCTGGCCAGAAGCGTG
CTGAGCAAGGCCAAGATCCTGCTGCTGGACGAGCCCAGCGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACAGCATCCAGAAGCTGCTGAACGAGAGGAGCCTGTTCCGGCAGGCC
ATCAGCCCCAGCGACAGGGTGAAGCTGTTCCCCCACCGGAACAGCAGCAAGTGCAAGAGC
AAGCCCCAGATCGCCGCCCTGAAGGAGGAGACCGAGGAGGAGGTGCAGGACACCAGGCTG
TAG
(pARM2268)
SEQ ID NO: 38
ATGCAGAGGTCGCCTCTGGAGAAGGCCAGCGTGGTGTCCAAGCTGTTCTTCAGCTGGACC
AGACCAATCCTGAGGAAGGGCTACAGACAGCGCCTGGAGCTGTCAGACATCTACCAGATC
CCTTCTGTGGACTCTGCTGACAACCTGTCTGAGAAGCTGGAGAGAGAGTGGGACAGAGAG
CTGGCCAGCAAGAAGAACCCTAAGCTGATCAACGCCCTGCGGAGGTGCTTCTTCTGGAGA
TTCATGTTCTACGGAATCTTCCTGTACCTGGGGGAGGTGACCAAGGCCGTGCAGCCTCTG
CTGCTGGGAAGAATCATCGCCTCCTACGACCCCGACAACAAGGAGGAGCGCTCTATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGTATCGGACAGCTG
GTGAGTCTGCTGTCCAACAACCTGAACAAGTTCGACGAGGGACTGGCCCTGGCCCACTTC
GTGTGGATCGCTCCTCTGCAGGTGGCCCTGCTGATGGGGCTGATCTGGGAGCTGCTGCAG
GCCTCTGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCTCAGAGATGATCGAGAACATCCAGTCTGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCTCAGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCTCTAACGGCGACGACAGCCTGTTCTTCAGCAACTTCAGCCTGCTGGGCACCCCTGTG
CTGAAGGACATCAACTTCAAGATCGAGAGAGGACAGCTGCTGGCCGTGGCCGGATCCACC
GGAGCCGGCAAGACCTCACTGCTGATGGTGATCATGGGAGAGCTGGAGCCTTCAGAGGGC
AAGATCAAGCACAGTGGAAGAATCTCATTCTGCTCTCAGTTCTCCTGGATCATGCCTGGC
ACCATCAAGGAGAACATCATCTTCGGTGTGTCCTACGACGAGTACAGATACAGAAGCGTG
ATCAAGGCCTGCCAGCTGGAGGAGGACATCTCCAAGTTCGCAGAGAAGGACAACATCGTG
CTGGGAGAGGGTGGCATCACCCTGAGCGGAGGCCAGAGGGCCAGAATCTCTCTGGCAAGA
GCAGTGTACAAGGACGCTGACCTGTACCTGCTGGACTCTCCTTTCGGATACCTGGACGTG
CTGACCGAGAAGGAGATCTTCGAGAGCTGCGTGTGCAAGCTGATGGCCAACAAGACCAGG
ATCCTGGTGACCTCTAAGATGGAGCACCTGAAGAAGGCTGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACTCTTTCGACCAGTTCAGCGCCGAGAGAAGAAACAGG
CATCCTGACCGAGACCCTGCACAGGTTCAGCCTGGAGGGCGACGCCCCTGTTCCTGGACC
GAGACCAAGAAGCAGTCTTTCAAGCAGACCGGAGAGTTCGGCGAGAAGAGGAAGAACTCT
ATCCTGAACCCAATCAACTCTATCAGGAAGTTCTCCATCGTGCAGAAGACCCCCCTGCAG
ATGAACGGCATCGAGGAGGACTCTGACGAGCCTCTGGAGAGAAGGCTGTCCCTGGTGCCA
GACTCTGAGCAGGGCGAGGCCATCCTGCCTCGCATCAGCGTGATCAGCACCGGCCCCACC
CTGCAGGCCAGGAGGAGGCAGTCTGTGCTGAACCTGATGACCCACAGCGTGAACCAGGGC
CAGAACATCCACAGGAAGACCACCGCCTCCACCAGGAAGGTGAGCCTGGCCCCTCAGGCC
AACCTGACCGAGCTGGACATCTACAGCAGAAGGCTGTCTCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCTCTCTGGTGGTG
CTGTGGCTGCTGGGCAACACCCCTCTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCACACC
CTGATCACCGTGAGCAAGATCCTGCACCACAAGATGCTGCACTCTGTGCTGCAGGCCCCT
ATGAGCACCCTGAACACCCTGAAGGCCGGTGGGATCCTGAACAGATTCTCCAAGGACATC
GCCATCCTGGACGACCTGCTGCCTCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGTCTGAGGGCAGGAGTCCAATCTTCACCCACCTGGTGACCAGCCTGAAG
GGACTGTGGACCCTGAGGGCCTTCGGCCGGCAGCCTTACTTCGAGACCCTGTTCCACAAG
GCTCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCTCCATCCTG
ACCTACCCCTACGACGTGCCCGACTACGCCACCGGCGAGGGAGAGGGAAGAGTGGGCATC
ATCCTGACCCTGGCCATGAACATCATGAGCACCCTGCAGTGGGCTGTGAACTCCAGCATC
GACGTGGACAGCCTGATGAGGTCTGTGAGCAGGGTGTTCAAGTTCATCGACATGCCAACC
GAGGGCAAGCCTACCAAGAGCACCAAGCCATACAAGAACGGCCAGCTGAGCAAGGTGATG
ATCATCGAGAACAGCCACGTGAAGAAGGACGACATCTGGCCCAGCGGCGGCCAGATGACC
GTGAAGGACCTGACCGCCAAGTACACCGAGGGCGGCAACGCCATCCTGGAGAACATCTCC
TTCTCAATCAGCCCTGGCCAGAGGGTGGGCCTGCTGGGAAGAACCGGCTCAGGCAAGAGC
ACCCTGCTGAGCGCCTTCCTGAGACTGCTGAACACCGAGGGCGAGATCCAGATCGACGGC
GTGTCTTGGGACTCAATCACCCTGCAGCAGTGGAGGAAGGCCTTCGGCGTGATCCCACAG
AAGGTGTTCATCTTCTCTGGAACCTTCAGAAAGAACCTGGACCCCTACGAGCAGTGGAGC
GACCAGGAGATCTGGAAGGTGGCCGACGAGGTGGGCCTGAGATCTGTGATCGAGCAGTTC
CCTGGCAAGCTGGACTTCGTGCTGGTGGACGGGGGCTGCGTGCTGAGCCACGGCCACAAG
CAGCTGATGTGCCTGGCCAGATCTGTGCTGAGCAAGGCCAAGATCCTGCTGCTGGACGAG
CCCAGTGCCCACCTGGACCCAGTGACCTACCAGATCATCAGAAGAACCCTGAAGCAGGCC
TTCGCCGACTGCACCGTGATCCTGTGCGAGCACAGGATCGAGGCCATGCTGGAGTGCCAG
CAGTTCCTGGTGATCGAGGAGAACAAGGTGCGGCAGTACGACTCCATCCAGAAGCTGCTG
AACGAGAGGAGCCTGTTCCGGCAGGCCATCAGCCCCTCCGACAGGGTGAAGCTGTTCCCC
CACCGGAACAGCAGCAAGTGCAAGTCTAAGCCCCAGATCGCCGCCCTGAAGGAGGAGACC
GAGGAGGAGGTGCAGGACACCAGGCTGTAG
(pARM2269)
SEQ ID NO: 39
ATGCAGAGGTCGCCTCTGGAGAAGGCCAGCGTGGTGTCCAAGCTGTTCTTCAGCTGGACC
AGACCAATCCTGAGGAAGGGCTACAGACAGCGCCTGGAGCTGTCAGACATCTACCAGATC
CCTTCTGTGGACTCTGCTGACAACCTGTCTGAGAAGCTGGAGAGAGAGTGGGACAGAGAG
CTGGCCAGCAAGAAGAACCCTAAGCTGATCAACGCCCTGCGGAGGTGCTTCTTCTGGAGA
TTCATGTTCTACGGAATCTTCCTGTACCTGGGGGAGGTGACCAAGGCCGTGCAGCCTCTG
CTGCTGGGAAGAATCATCGCCTCCTACGACCCCGACAACAAGGAGGAGCGCTCTATCGCC
ATCTACCTGGGCATCGGCCTGTGCCTGCTGTTCATCGTGAGGACCCTGCTGCTGCACCCA
GCCATCTTCGGCCTGCACCACATCGGAATGCAGATGAGAATCGCCATGTTCAGCCTGATC
TACAAGAAGACCCTGAAGCTGTCAAGCAGGGTGCTGGACAAGATCAGTATCGGACAGCTG
GTGAGTCTGCTGTCCAACAACCTGAACAAGTTCGACGAGGGACTGGCCCTGGCCCACTTC
GTGTGGATCGCTCCTCTGCAGGTGGCCCTGCTGATGGGGCTGATCTGGGAGCTGCTGCAG
GCCTCTGCCTTCTGCGGCCTGGGCTTCCTGATCGTGCTGGCCCTGTTCCAGGCCGGCCTG
GGCAGAATGATGATGAAGTACAGAGACCAGAGAGCTGGCAAGATCAGCGAGAGACTGGTG
ATCACCTCAGAGATGATCGAGAACATCCAGTCTGTGAAGGCATACTGCTGGGAGGAGGCC
ATGGAGAAGATGATCGAGAACCTGAGACAGACCGAGCTGAAGCTGACCCGGAAGGCCGCC
TACGTGAGATACTTCAACAGCAGCGCCTTCTTCTTCTCAGGGTTCTTCGTGGTGTTCCTG
TCTGTGCTGCCCTACGCCCTGATCAAGGGCATCATCCTGCGGAAGATCTTCACCACCATC
TCATTCTGCATCGTGCTGCGCATGGCCGTGACCCGGCAGTTCCCCTGGGCCGTGCAGACC
TGGTACGACTCTCTGGGAGCCATCAACAAGATCCAGGACTTCCTGCAGAAGCAGGAGTAC
AAGACCCTGGAGTACAACCTGACCACCACCGAGGTGGTGATGGAGAACGTGACCGCCTTC
TGGGAGGAGGGATTCGGCGAGCTGTTCGAGAAGGCCAAGCAGAACAACAACAACAGAAAG
ACCTCTAACGGCGACGACAGCCTGTTCTTCAGCAACTTCAGCCTGCTGGGCACCCCTGTG
CTGAAGGACATCAACTTCAAGATCGAGAGAGGACAGCTGCTGGCCGTGGCCGGATCCACC
GGAGCCGGCAAGACCTCACTGCTGATGGTGATCATGGGAGAGCTGGAGCCTTCAGAGGGC
AAGATCAAGCACAGTGGAAGAATCTCATTCTGCTCTCAGTTCTCCTGGATCATGCCTGGC
ACCATCAAGGAGAACATCATCTTCGGTGTGTCCTACGACGAGTACAGATACAGAAGCGTG
ATCAAGGCCTGCCAGCTGGAGGAGGACATCTCCAAGTTCGCAGAGAAGGACAACATCGTG
CTGGGAGAGGGTGGCATCACCCTGAGCGGAGGCCAGAGGGCCAGAATCTCTCTGGCAAGA
GCAGTGTACAAGGACGCTGACCTGTACCTGCTGGACTCTCCTTTCGGATACCTGGACGTG
CTGACCGAGAAGGAGATCTTCGAGAGCTGCGTGTGCAAGCTGATGGCCAACAAGACCAGG
ATCCTGGTGACCTCTAAGATGGAGCACCTGAAGAAGGCTGACAAGATCCTGATCCTGCAC
GAGGGCAGCAGCTACTTCTACGGGACCTTCAGCGAGCTGCAGAACCTGCAGCCAGACTTC
AGCAGCAAGCTGATGGGCTGCGACTCTTTCGACCAGTTCAGCGCCGAGAGAAGAAACAGC
ATCCTGACCGAGACCCTGCACAGGTTCAGCCTGGAGGGCGACGCCCCTGTGTCCTGGACC
GAGACCAAGAAGCAGTCTTTCAAGCAGACCGGAGAGTTCGGCGAGAAGAGGAAGAACTCT
ATCCTGAACCCAATCAACTCTATCAGGAAGTTCTCCATCGTGCAGAAGACCCCCCTGCAG
ATGAACGGCATCGAGGAGGACTCTGACGAGCCTCTGGAGAGAAGGCTGTCCCTGGTGCCA
GACTCTGAGCAGGGCGAGGCCATCCTGCCTCGCATCAGCGTGATCAGCACCGGCCCCACC
CTGCAGGCCAGGAGGAGGCAGTCTGTGCTGAACCTGATGACCCACAGCGTGAACCAGGGC
CAGAACATCCACAGGAAGACCACCGCCTCCACCAGGAAGGTGAGCCTGGCCCCTCAGGCC
AACCTGACCGAGCTGGACATCTACAGCAGAAGGCTGTCTCAGGAGACCGGCCTGGAGATC
AGCGAGGAGATCAACGAGGAGGACCTGAAGGAGTGCTTCTTCGACGACATGGAGAGCATC
CCAGCCGTGACCACCTGGAACACCTACCTGAGGTACATCACCGTGCACAAGAGCCTGATC
TTCGTGCTGATCTGGTGCCTGGTGATCTTCCTGGCCGAGGTGGCCGCCTCTCTGGTGGTG
CTGTGGCTGCTGGGCAACACCCCTCTGCAGGACAAGGGCAACAGCACCCACAGCAGAAAC
AACAGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCACACC
CTGATCACCGTGAGCAAGATCCTGCACCACAAGATGCTGCACTCTGTGCTGCAGGCCCCT
ATGAGCACCCTGAACACCCTGAAGGCCGGTGGGATCCTGAACAGATTCTCCAAGGACATC
GCCATCCTGGACGACCTGCTGCCTCTGACCATCTTCGACTTCATCCAGCTGCTGCTGATC
GTGATCGGAGCCATCGCCGTGGTGGCCGTGCTGCAGCCCTACATCTTCGTGGCCACCGTG
CCAGTGATCGTGGCCTTCATCATGCTGAGAGCCTACTTCCTGCAGACCAGCCAGCAGCTG
AAGCAGCTGGAGTCTGAGGGCAGGAGTCCAATCTTCACCCACCTGGTGACCAGCCTGAAG
GGACTGTGGACCCTGAGGGCCTTCGGCCGGCAGCCTTACTTCGAGACCCTGTTCCACAAG
GCTCTGAACCTGCACACCGCCAACTGGTTCCTGTACCTGAGCACCCTGCGCTGGTTCCAG
ATGAGAATCGAGATGATCTTCGTGATCTTCTTCATCGCCGTGACCTTCATCTCCATCCTG
ACCACCGGCGAGGGAGAGGGAAGAGTGGGCATCATCCTGACCCTGGCCATGAACATCATG
AGCACCCTGCAGTGGGCTGTGAACTCCAGCATCGACGTGGACAGCCTGATGAGGTCTGTG
AGCAGGGTGTTCAAGTTCATCGACATGCCAACCGAGGGCAAGCCTACCAAGAGCACCAAG
CCATACAAGAACGGCCAGCTGAGCAAGGTGATGATCATCGAGAACAGCCACGTGAAGAAG
GACGACATCTGGCCCAGCGGCGGCCAGATGACCGTGAAGGACCTGACCGCCAAGTACACC
GAGGGCGGCAACGCCATCCTGGAGAACATCTCCTTCTCAATCAGCCCTGGCCAGAGGGTG
GGCCTGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCTGCAG
CAGTGGAGGAAGGCCTTCGGCGTGATCCCACAGAAGGTGTTCATCTTCTCTGGAACCTTC
AGAAAGAACCTGGACCCCTACGAGCAGTGGAGCGACCAGGAGATCTGGAAGGTGGCCGAC
GAGGTGGGCCTGAGATCTGTGATCGAGCAGTTCCCTGGCAAGCTGGACTTCGTGCTGGTG
GACGGGGGCTGCGTGCTGAGCCACGGCCACAAGCAGCTGATGTGCCTGGCCAGATCTGTG
CTGAGCAAGGCCAAGATCCTGCTGCTGGACGAGCCCAGTGCCCACCTGGACCCAGTGACC
TACCAGATCATCAGAAGAACCCTGAAGCAGGCCTTCGCCGACTGCACCGTGATCCTGTGC
GAGCACAGGATCGAGGCCATGCTGGAGTGCCAGCAGTTCCTGGTGATCGAGGAGAACAAG
GTGCGGCAGTACGACTCCATCCAGAAGCTGCTGAACGAGAGGAGCCTGTTCCGGCAGGCC
ATCAGCCCCTCCGACAGGGTGAAGCTGTTCCCCCACCGGAACAGCAGCAAGTGCAAGTCT
AAGCCCCAGATCGCCGCCCTGAAGGAGGAGACCGAGGAGGAGGTGCAGGACACCAGGCTG
TACCCCTACGACGTGCCCGACTACGCCTAG
(pARM2381)
SEQ ID NO: 40
ATGCAGAGGTCGCCTCTGGAGAAGGCCAGCGTGGTGTCCAAGCTGTTCTTCAGCTGG
ACCAGACCAATCCTGAGGAAGGGCTACAGACAGCGCCTGGAGCTGTCAGACATCTA
CCAGATCCCTTCTGTGGACTCTGCTGACAACCTGTCTGAGAAGCTGGAGAGAGAGTG
GGACAGAGAGCTGGCCAGCAAGAAGAACCCTAAGCTGATCAACGCCCTGCGGAGGT
GCTTCTTCTGGAGATTCATGTTCTACGGAATCTTCCTGTACCTGGGGGAGGTGACCA
AGGCCGTGCAGCCTCTGCTGCTGGGAAGAATCATCGCCTCCTACGACCCCGACAAC
AAGGAGGAGCGCTCTATCGCCATCTACCTGGGCATCGGCCTGTGCCTGCTGTTCATC
GTGAGGACCCTGCTGCTGCACCCAGCCATCTTCGGCCTGCACCACATCGGAATGCAG
ATGAGAATCGCCATGTTCAGCCTGATCTACAAGAAGACCCTGAAGCTGTCAAGCAG
GGTGCTGGACAAGATCAGTATCGGACAGCTGGTGAGTCTGCTGTCCAACAACCTGA
ACAAGTTCGACGAGGGACTGGCCCTGGCCCACTTCGTGTGGATCGCTCCTCTGCAGG
TGGCCCTGCTGATGGGGCTGATCTGGGAGCTGCTGCAGGCCTCTGCCTTCTGCGGCC
TGGGCTTCCTGATCGTGCTGGCCCTGTTCCAGGCCGGCCTGGGCAGAATGATGATGA
AGTACAGAGACCAGAGAGCTGGCAAGATCAGCGAGAGACTGGTGATCACCTCAGAG
ATGATCGAGAACATCCAGTCTGTGAAGGCATACTGCTGGGAGGAGGCCATGGAGAA
GATGATCGAGAACCTGAGACAGACCGAGCTGAAGCTGACCCGGAAGGCCGCCTACG
TGAGATACTTCAACAGCAGCGCCTTCTTCTTCTCAGGGTTCTTCGTGGTGTTCCTGTC
TGTGCTGCCCTACGCCCTGATCAAGGGCATCATCCTGCGGAAGATCTTCACCACCAT
CTCATTCTGCATCGTGCTGCGCATGGCCGTGACCCGGCAGTTCCCCTGGGCCGTGCA
GACCTGGTACGACTCTCTGGGAGCCATCAACAAGATCCAGGACTTCCTGCAGAAGC
AGGAGTACAAGACCCTGGAGTACAACCTGACCACCACCGAGGTGGTGATGGAGAAC
GTGACCGCCTTCTGGGAGGAGGGATTCGGCGAGCTGTTCGAGAAGGCCAAGCAGAA
CAACAACAACAGAAAGACCTCTAACGGCGACGACAGCCTGTTCTTCAGCAACTTCA
GCCTGCTGGGCACCCCTGTGCTGAAGGACATCAACTTCAAGATCGAGAGAGGACAG
CTGCTGGCCGTGGCCGGATCCACCGGAGCCGGCAAGACCTCACTGCTGATGGTGAT
CATGGGAGAGCTGGAGCCTTCAGAGGGCAAGATCAAGCACAGTGGAAGAATCTCAT
TCTGCTCTCAGTTCTCCTGGATCATGCCTGGCACCATCAAGGAGAACATCATCTTCG
GTGTGTCCTACGACGAGTACAGATACAGAAGCGTGATCAAGGCCTGCCAGCTGGAG
GAGGACATCTCCAAGTTCGCAGAGAAGGACAACATCGTGCTGGGAGAGGGTGGCAT
CACCCTGAGCGGAGGCCAGAGGGCCAGAATCTCTCTGGCAAGAGCAGTGTACAAGG
ACGCTGACCTGTACCTGCTGGACTCTCCTTTCGGATACCTGGACGTGCTGACCGAGA
AGGAGATCTTCGAGAGCTGCGTGTGCAAGCTGATGGCCAACAAGACCAGGATCCTG
GTGACCTCTAAGATGGAGCACCTGAAGAAGGCTGACAAGATCCTGATCCTGCACGA
GGGCAGCAGCTACTTCTACGGGACCTTCAGCGAGCTGCAGAACCTGCAGCCAGACT
TCAGCAGCAAGCTGATGGGCTGCGACTCTTTCGACCAGTTCAGCGCCGAGAGAAGA
AACAGCATCCTGACCGAGACCCTGCACAGGTTCAGCCTGGAGGGCGACGCCCCTGT
GTCCTGGACCGAGACCAAGAAGCAGTCTTTCAAGCAGACCGGAGAGTTCGGCGAGA
AGAGGAAGAACTCTATCCTGAACCCAATCAACTCTATCAGGAAGTTCTCCATCGTGC
AGAAGACCCCCCTGCAGATGAACGGCATCGAGGAGGACTCTGACGAGCCTCTGGAG
AGAAGGCTGTCCCTGGTGCCAGACTCTGAGCAGGGCGAGGCCATCCTGCCTCGCAT
CAGCGTGATCAGCACCGGCCCCACCCTGCAGGCCAGGAGGAGGCAGTCTGTGCTGA
ACCTGATGACCCACAGCGTGAACCAGGGCCAGAACATCCACAGGAAGACCACCGCC
TCCACCAGGAAGGTGAGCCTGGCCCCTCAGGCCAACCTGACCGAGCTGGACATCTA
CAGCAGAAGGCTGTCTCAGGAGACCGGCCTGGAGATCAGCGAGGAGATCAACGAG
GAGGACCTGAAGGAGTGCTTCTTCGACGACATGGAGAGCATCCCAGCCGTGACCAC
CTGGAACACCTACCTGAGGTACATCACCGTGCACAAGAGCCTGATCTTCGTGCTGAT
CTGGTGCCTGGTGATCTTCCTGGCCGAGGTGGCCGCCTCTCTGGTGGTGCTGTGGCT
GCTGGGCAACACCCCTCTGCAGGACAAGGGCAACAGCACCCACAGCAGAAACAAC
AGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCAC
ACCCTGATCACCGTGAGCAAGATCCTGCACCACAAGATGCTGCACTCTGTGCTGCAG
GCCCCTATGAGCACCCTGAACACCCTGAAGGCCGGTGGGATCCTGAACAGATTCTCC
AAGGACATCGCCATCCTGGACGACCTGCTGCCTCTGACCATCTTCGACTTCATCCAG
CTGCTGCTGATCGTGATCGGAGCCATCGCCGTGGTGGCCGTGCTGCAGCCCTACATC
TTCGTGGCCACCGTGCCAGTGATCGTGGCCTTCATCATGCTGAGAGCCTACTTCCTG
CAGACCAGCCAGCAGCTGAAGCAGCTGGAGTCTGAGGGCAGGAGTCCAATCTTCAC
CCACCTGGTGACCAGCCTGAAGGGACTGTGGACCCTGAGGGCCTTCGGCCGGCAGC
CTTACTTCGAGACCCTGTTCCACAAGGCTCTGAACCTGCACACCGCCAACTGGTTCC
TGTACCTGAGCACCCTGCGCTGGTTCCAGATGAGAATCGAGATGATCTTCGTGATCT
TCTTCATCGCCGTGACCTTCATCTCCATCCTGACCACCGGCGAGGGAGAGGGAAGAG
TGGGCATCATCCTGACCCTGGCCATGAACATCATGAGCACCCTGCAGTGGGCTGTGA
ACTCCAGCATCGACGTGGACAGCCTGATGAGGTCTGTGAGCAGGGTGTTCAAGTTCA
TCGACATGCCAACCGAGGGCAAGCCTACCAAGAGCACCAAGCCATACAAGAACGGC
CAGCTGAGCAAGGTGATGATCATCGAGAACAGCCACGTGAAGAAGGACGACATCTG
GCCCAGCGGCGGCCAGATGACCGTGAAGGACCTGACCGCCAAGTACACCGAGGGCG
GCAACGCCATCCTGGAGAACATCTCCTTCTCAATCAGCCCTGGCCAGAGGGTGGGCC
TGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCT
GCAGCAGTGGAGGAAGGCCTTCGGCGTGATCCCACAGAAGGTGTTCATCTTCTCTGG
AACCTTCAGAAAGAACCTGGACCCCTACGAGCAGTGGAGCGACCAGGAGATCTGGA
AGGTGGCCGACGAGGTGGGCCTGAGATCTGTGATCGAGCAGTTCCCTGGCAAGCTG
GACTTCGTGCTGGTGGACGGGGGCTGCGTGCTGAGCCACGGCCACAAGCAGCTGAT
GTGCCTGGCCAGATCTGTGCTGAGCAAGGCCAAGATCCTGCTGCTGGACGAGCCCA
GTGCCCACCTGGACCCAGTGACCTACCAGATCATCAGAAGAACCCTGAAGCAGGCC
TTCGCCGACTGCACCGTGATCCTGTGCGAGCACAGGATCGAGGCCATGCTGGAGTG
CCAGCAGTTCCTGGTGATCGAGGAGAACAAGGTGCGGCAGTACGACTCCATCCAGA
AGCTGCTGAACGAGAGGAGCCTGTTCCGGCAGGCCATCAGCCCCTCCGACAGGGTG
AAGCTGTTCCCCCACCGGAACAGCAGCAAGTGCAAGTCTAAGCCCCAGATCGCCGC
CCTGAAGGAGGAGACCGAGGAGGAGGTGCAGGACACCAGGCTGGACTACAAGGAC
GATGACGATAAGTAG
(pARM2382)
SEQ ID NO: 41
ATGCAGAGGTCGCCTCTGGAGAAGGCCAGCGTGGTGTCCAAGCTGTTCTTCAGCTGG
ACCAGACCAATCCTGAGGAAGGGCTACAGACAGCGCCTGGAGCTGTCAGACATCTA
CCAGATCCCTTCTGTGGACTCTGCTGACAACCTGTCTGAGAAGCTGGAGAGAGAGTG
GGACAGAGAGCTGGCCAGCAAGAAGAACCCTAAGCTGATCAACGCCCTGCGGAGGT
GCTTCTTCTGGAGATTCATGTTCTACGGAATCTTCCTGTACCTGGGGGAGGTGACCA
AGGCCGTGCAGCCTCTGCTGCTGGGAAGAATCATCGCCTCCTACGACCCCGACAAC
AAGGAGGAGCGCTCTATCGCCATCTACCTGGGCATCGGCCTGTGCCTGCTGTTCATC
GTGAGGACCCTGCTGCTGCACCCAGCCATCTTCGGCCTGCACCACATCGGAATGCAG
ATGAGAATCGCCATGTTCAGCCTGATCTACAAGAAGACCCTGAAGCTGTCAAGCAG
GGTGCTGGACAAGATCAGTATCGGACAGCTGGTGAGTCTGCTGTCCAACAACCTGA
ACAAGTTCGACGAGGGACTGGCCCTGGCCCACTTCGTGTGGATCGCTCCTCTGCAGG
TGGCCCTGCTGATGGGGCTGATCTGGGAGCTGCTGCAGGCCTCTGCCTTCTGCGGCC
TGGGCTTCCTGATCGTGCTGGCCCTGTTCCAGGCCGGCCTGGGCAGAATGATGATGA
AGTACAGAGACCAGAGAGCTGGCAAGATCAGCGAGAGACTGGTGATCACCTCAGAG
ATGATCGAGAACATCCAGTCTGTGAAGGCATACTGCTGGGAGGAGGCCATGGAGAA
GATGATCGAGAACCTGAGACAGACCGAGCTGAAGCTGACCCGGAAGGCCGCCTACG
TGAGATACTTCAACAGCAGCGCCTTCTTCTTCTCAGGGTTCTTCGTGGTGTTCCTGTC
TGTGCTGCCCTACGCCCTGATCAAGGGCATCATCCTGCGGAAGATCTTCACCACCAT
CTCATTCTGCATCGTGCTGCGCATGGCCGTGACCCGGCAGTTCCCCTGGGCCGTGCA
GACCTGGTACGACTCTCTGGGAGCCATCAACAAGATCCAGGACTTCCTGCAGAAGC
AGGAGTACAAGACCCTGGAGTACAACCTGACCACCACCGAGGTGGTGATGGAGAAC
GTGACCGCCTTCTGGGAGGAGGGATTCGGCGAGCTGTTCGAGAAGGCCAAGCAGAA
CAACAACAACAGAAAGACCTCTAACGGCGACGACAGCCTGTTCTTCAGCAACTTCA
GCCTGCTGGGCACCCCTGTGCTGAAGGACATCAACTTCAAGATCGAGAGAGGACAG
CTGCTGGCCGTGGCCGGATCCACCGGAGCCGGCAAGACCTCACTGCTGATGGTGAT
CATGGGAGAGCTGGAGCCTTCAGAGGGCAAGATCAAGCACAGTGGAAGAATCTCAT
TCTGCTCTCAGTTCTCCTGGATCATGCCTGGCACCATCAAGGAGAACATCATCTTCG
GTGTGTCCTACGACGAGTACAGATACAGAAGCGTGATCAAGGCCTGCCAGCTGGAG
GAGGACATCTCCAAGTTCGCAGAGAAGGACAACATCGTGCTGGGAGAGGGTGGCAT
CACCCTGAGCGGAGGCCAGAGGGCCAGAATCTCTCTGGCAAGAGCAGTGTACAAGG
ACGCTGACCTGTACCTGCTGGACTCTCCTTTCGGATACCTGGACGTGCTGACCGAGA
AGGAGATCTTCGAGAGCTGCGTGTGCAAGCTGATGGCCAACAAGACCAGGATCCTG
GTGACCTCTAAGATGGAGCACCTGAAGAAGGCTGACAAGATCCTGATCCTGCACGA
GGGCAGCAGCTACTTCTACGGGACCTTCAGCGAGCTGCAGAACCTGCAGCCAGACT
TCAGCAGCAAGCTGATGGGCTGCGACTCTTTCGACCAGTTCAGCGCCGAGAGAAGA
AACAGCATCCTGACCGAGACCCTGCACAGGTTCAGCCTGGAGGGCGACGCCCCTGT
GTCCTGGACCGAGACCAAGAAGCAGTCTTTCAAGCAGACCGGAGAGTTCGGCGAGA
AGAGGAAGAACTCTATCCTGAACCCAATCAACTCTATCAGGAAGTTCTCCATCGTGC
AGAAGACCCCCCTGCAGATGAACGGCATCGAGGAGGACTCTGACGAGCCTCTGGAG
AGAAGGCTGTCCCTGGTGCCAGACTCTGAGCAGGGCGAGGCCATCCTGCCTCGCAT
CAGCGTGATCAGCACCGGCCCCACCCTGCAGGCCAGGAGGAGGCAGTCTGTGCTGA
ACCTGATGACCCACAGCGTGAACCAGGGCCAGAACATCCACAGGAAGACCACCGCC
TCCACCAGGAAGGTGAGCCTGGCCCCTCAGGCCAACCTGACCGAGCTGGACATCTA
CAGCAGAAGGCTGTCTCAGGAGACCGGCCTGGAGATCAGCGAGGAGATCAACGAG
GAGGACCTGAAGGAGTGCTTCTTCGACGACATGGAGAGCATCCCAGCCGTGACCAC
CTGGAACACCTACCTGAGGTACATCACCGTGCACAAGAGCCTGATCTTCGTGCTGAT
CTGGTGCCTGGTGATCTTCCTGGCCGAGGTGGCCGCCTCTCTGGTGGTGCTGTGGCT
GCTGGGCAACACCCCTCTGCAGGACAAGGGCAACAGCACCCACAGCAGAAACAAC
AGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCAC
ACCCTGATCACCGTGAGCAAGATCCTGCACCACAAGATGCTGCACTCTGTGCTGCAG
GCCCCTATGAGCACCCTGAACACCCTGAAGGCCGGTGGGATCCTGAACAGATTCTCC
AAGGACATCGCCATCCTGGACGACCTGCTGCCTCTGACCATCTTCGACTTCATCCAG
CTGCTGCTGATCGTGATCGGAGCCATCGCCGTGGTGGCCGTGCTGCAGCCCTACATC
TTCGTGGCCACCGTGCCAGTGATCGTGGCCTTCATCATGCTGAGAGCCTACTTCCTG
CAGACCAGCCAGCAGCTGAAGCAGCTGGAGTCTGAGGGCAGGAGTCCAATCTTCAC
CCACCTGGTGACCAGCCTGAAGGGACTGTGGACCCTGAGGGCCTTCGGCCGGCAGC
CTTACTTCGAGACCCTGTTCCACAAGGCTCTGAACCTGCACACCGCCAACTGGTTCC
TGTACCTGAGCACCCTGCGCTGGTTCCAGATGAGAATCGAGATGATCTTCGTGATCT
TCTTCATCGCCGTGACCTTCATCTCCATCCTGACCACCGGCGAGGGAGAGGGAAGAG
TGGGCATCATCCTGACCCTGGCCATGAACATCATGAGCACCCTGCAGTGGGCTGTGA
ACTCCAGCATCGACGTGGACAGCCTGATGAGGTCTGTGAGCAGGGTGTTCAAGTTCA
TCGACATGCCAACCGAGGGCAAGCCTACCAAGAGCACCAAGCCATACAAGAACGGC
CAGCTGAGCAAGGTGATGATCATCGAGAACAGCCACGTGAAGAAGGACGACATCTG
GCCCAGCGGCGGCCAGATGACCGTGAAGGACCTGACCGCCAAGTACACCGAGGGCG
GCAACGCCATCCTGGAGAACATCTCCTTCTCAATCAGCCCTGGCCAGAGGGTGGGCC
TGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCT
GCAGCAGTGGAGGAAGGCCTTCGGCGTGATCCCACAGAAGGTGTTCATCTTCTCTGG
AACCTTCAGAAAGAACCTGGACCCCTACGAGCAGTGGAGCGACCAGGAGATCTGGA
AGGTGGCCGACGAGGTGGGCCTGAGATCTGTGATCGAGCAGTTCCCTGGCAAGCTG
GACTTCGTGCTGGTGGACGGGGGCTGCGTGCTGAGCCACGGCCACAAGCAGCTGAT
GTGCCTGGCCAGATCTGTGCTGAGCAAGGCCAAGATCCTGCTGCTGGACGAGCCCA
GTGCCCACCTGGACCCAGTGACCTACCAGATCATCAGAAGAACCCTGAAGCAGGCC
TTCGCCGACTGCACCGTGATCCTGTGCGAGCACAGGATCGAGGCCATGCTGGAGTG
CCAGCAGTTCCTGGTGATCGAGGAGAACAAGGTGCGGCAGTACGACTCCATCCAGA
AGCTGCTGAACGAGAGGAGCCTGTTCCGGCAGGCCATCAGCCCCTCCGACAGGGTG
AAGCTGTTCCCCCACCGGAACAGCAGCAAGTGCAAGTCTAAGCCCCAGATCGCCGC
CCTGAAGGAGGAGACCGAGGAGGAGGTGCAGGACACCAGGCTGGGCGGCGGCAGC
GGCGAGCAGAAACTGATCAGCGAAGAGGATCTGAACGGCGGCGGCAGCGGCGAGC
AGAAACTGATCAGCGAAGAGGATCTGAACGGCGGCGGCAGCGGCGAGCAGAAACT
GATCAGCGAAGAGGATCTGAACTAG
(pARM2383)
SEQ ID NO: 42
ATGCAGAGGTCGCCTCTGGAGAAGGCCAGCGTGGTGTCCAAGCTGTTCTTCAGCTGG
ACCAGACCAATCCTGAGGAAGGGCTACAGACAGCGCCTGGAGCTGTCAGACATCTA
CCAGATCCCTTCTGTGGACTCTGCTGACAACCTGTCTGAGAAGCTGGAGAGAGAGTG
GGACAGAGAGCTGGCCAGCAAGAAGAACCCTAAGCTGATCAACGCCCTGCGGAGGT
GCTTCTTCTGGAGATTCATGTTCTACGGAATCTTCCTGTACCTGGGGGAGGTGACCA
AGGCCGTGCAGCCTCTGCTGCTGGGAAGAATCATCGCCTCCTACGACCCCGACAAC
AAGGAGGAGCGCTCTATCGCCATCTACCTGGGCATCGGCCTGTGCCTGCTGTTCATC
GTGAGGACCCTGCTGCTGCACCCAGCCATCTTCGGCCTGCACCACATCGGAATGCAG
ATGAGAATCGCCATGTTCAGCCTGATCTACAAGAAGACCCTGAAGCTGTCAAGCAG
GGTGCTGGACAAGATCAGTATCGGACAGCTGGTGAGTCTGCTGTCCAACAACCTGA
ACAAGTTCGACGAGGGACTGGCCCTGGCCCACTTCGTGTGGATCGCTCCTCTGCAGG
TGGCCCTGCTGATGGGGCTGATCTGGGAGCTGCTGCAGGCCTCTGCCTTCTGCGGCC
TGGGCTTCCTGATCGTGCTGGCCCTGTTCCAGGCCGGCCTGGGCAGAATGATGATGA
AGTACAGAGACCAGAGAGCTGGCAAGATCAGCGAGAGACTGGTGATCACCTCAGAG
ATGATCGAGAACATCCAGTCTGTGAAGGCATACTGCTGGGAGGAGGCCATGGAGAA
GATGATCGAGAACCTGAGACAGACCGAGCTGAAGCTGACCCGGAAGGCCGCCTACG
TGAGATACTTCAACAGCAGCGCCTTCTTCTTCTCAGGGTTCTTCGTGGTGTTCCTGTC
TGTGCTGCCCTACGCCCTGATCAAGGGCATCATCCTGCGGAAGATCTTCACCACCAT
CTCATTCTGCATCGTGCTGCGCATGGCCGTGACCCGGCAGTTCCCCTGGGCCGTGCA
GACCTGGTACGACTCTCTGGGAGCCATCAACAAGATCCAGGACTTCCTGCAGAAGC
AGGAGTACAAGACCCTGGAGTACAACCTGACCACCACCGAGGTGGTGATGGAGAAC
GTGACCGCCTTCTGGGAGGAGGGATTCGGCGAGCTGTTCGAGAAGGCCAAGCAGAA
CAACAACAACAGAAAGACCTCTAACGGCGACGACAGCCTGTTCTTCAGCAACTTCA
GCCTGCTGGGCACCCCTGTGCTGAAGGACATCAACTTCAAGATCGAGAGAGGACAG
CTGCTGGCCGTGGCCGGATCCACCGGAGCCGGCAAGACCTCACTGCTGATGGTGAT
CATGGGAGAGCTGGAGCCTTCAGAGGGCAAGATCAAGCACAGTGGAAGAATCTCAT
TCTGCTCTCAGTTCTCCTGGATCATGCCTGGCACCATCAAGGAGAACATCATCTTCG
GTGTGTCCTACGACGAGTACAGATACAGAAGCGTGATCAAGGCCTGCCAGCTGGAG
GAGGACATCTCCAAGTTCGCAGAGAAGGACAACATCGTGCTGGGAGAGGGTGGCAT
CACCCTGAGCGGAGGCCAGAGGGCCAGAATCTCTCTGGCAAGAGCAGTGTACAAGG
ACGCTGACCTGTACCTGCTGGACTCTCCTTTCGGATACCTGGACGTGCTGACCGAGA
AGGAGATCTTCGAGAGCTGCGTGTGCAAGCTGATGGCCAACAAGACCAGGATCCTG
GTGACCTCTAAGATGGAGCACCTGAAGAAGGCTGACAAGATCCTGATCCTGCACGA
GGGCAGCAGCTACTTCTACGGGACCTTCAGCGAGCTGCAGAACCTGCAGCCAGACT
TCAGCAGCAAGCTGATGGGCTGCGACTCTTTCGACCAGTTCAGCGCCGAGAGAAGA
AACAGCATCCTGACCGAGACCCTGCACAGGTTCAGCCTGGAGGGCGACGCCCCTGT
GTCCTGGACCGAGACCAAGAAGCAGTCTTTCAAGCAGACCGGAGAGTTCGGCGAGA
AGAGGAAGAACTCTATCCTGAACCCAATCAACTCTATCAGGAAGTTCTCCATCGTGC
AGAAGACCCCCCTGCAGATGAACGGCATCGAGGAGGACTCTGACGAGCCTCTGGAG
AGAAGGCTGTCCCTGGTGCCAGACTCTGAGCAGGGCGAGGCCATCCTGCCTCGCAT
CAGCGTGATCAGCACCGGCCCCACCCTGCAGGCCAGGAGGAGGCAGTCTGTGCTGA
ACCTGATGACCCACAGCGTGAACCAGGGCCAGAACATCCACAGGAAGACCACCGCC
TCCACCAGGAAGGTGAGCCTGGCCCCTCAGGCCAACCTGACCGAGCTGGACATCTA
CAGCAGAAGGCTGTCTCAGGAGACCGGCCTGGAGATCAGCGAGGAGATCAACGAG
GAGGACCTGAAGGAGTGCTTCTTCGACGACATGGAGAGCATCCCAGCCGTGACCAC
CTGGAACACCTACCTGAGGTACATCACCGTGCACAAGAGCCTGATCTTCGTGCTGAT
CTGGTGCCTGGTGATCTTCCTGGCCGAGGTGGCCGCCTCTCTGGTGGTGCTGTGGCT
GCTGGGCAACACCCCTCTGCAGGACAAGGGCAACAGCACCCACAGCAGAAACAAC
AGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCAC
ACCCTGATCACCGTGAGCAAGATCCTGCACCACAAGATGCTGCACTCTGTGCTGCAG
GCCCCTATGAGCACCCTGAACACCCTGAAGGCCGGTGGGATCCTGAACAGATTCTCC
AAGGACATCGCCATCCTGGACGACCTGCTGCCTCTGACCATCTTCGACTTCATCCAG
CTGCTGCTGATCGTGATCGGAGCCATCGCCGTGGTGGCCGTGCTGCAGCCCTACATC
TTCGTGGCCACCGTGCCAGTGATCGTGGCCTTCATCATGCTGAGAGCCTACTTCCTG
CAGACCAGCCAGCAGCTGAAGCAGCTGGAGTCTGAGGGCAGGAGTCCAATCTTCAC
CCACCTGGTGACCAGCCTGAAGGGACTGTGGACCCTGAGGGCCTTCGGCCGGCAGC
CTTACTTCGAGACCCTGTTCCACAAGGCTCTGAACCTGCACACCGCCAACTGGTTCC
TGTACCTGAGCACCCTGCGCTGGTTCCAGATGAGAATCGAGATGATCTTCGTGATCT
TCTTCATCGCCGTGACCTTCATCTCCATCCTGACCACCGGCGAGGGAGAGGGAAGAG
TGGGCATCATCCTGACCCTGGCCATGAACATCATGAGCACCCTGCAGTGGGCTGTGA
ACTCCAGCATCGACGTGGACAGCCTGATGAGGTCTGTGAGCAGGGTGTTCAAGTTCA
TCGACATGCCAACCGAGGGCAAGCCTACCAAGAGCACCAAGCCATACAAGAACGGC
CAGCTGAGCAAGGTGATGATCATCGAGAACAGCCACGTGAAGAAGGACGACATCTG
GCCCAGCGGCGGCCAGATGACCGTGAAGGACCTGACCGCCAAGTACACCGAGGGCG
GCAACGCCATCCTGGAGAACATCTCCTTCTCAATCAGCCCTGGCCAGAGGGTGGGCC
TGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCT
GCAGCAGTGGAGGAAGGCCTTCGGCGTGATCCCACAGAAGGTGTTCATCTTCTCTGG
AACCTTCAGAAAGAACCTGGACCCCTACGAGCAGTGGAGCGACCAGGAGATCTGGA
AGGTGGCCGACGAGGTGGGCCTGAGATCTGTGATCGAGCAGTTCCCTGGCAAGCTG
GACTTCGTGCTGGTGGACGGGGGCTGCGTGCTGAGCCACGGCCACAAGCAGCTGAT
GTGCCTGGCCAGATCTGTGCTGAGCAAGGCCAAGATCCTGCTGCTGGACGAGCCCA
GTGCCCACCTGGACCCAGTGACCTACCAGATCATCAGAAGAACCCTGAAGCAGGCC
TTCGCCGACTGCACCGTGATCCTGTGCGAGCACAGGATCGAGGCCATGCTGGAGTG
CCAGCAGTTCCTGGTGATCGAGGAGAACAAGGTGCGGCAGTACGACTCCATCCAGA
AGCTGCTGAACGAGAGGAGCCTGTTCCGGCAGGCCATCAGCCCCTCCGACAGGGTG
AAGCTGTTCCCCCACCGGAACAGCAGCAAGTGCAAGTCTAAGCCCCAGATCGCCGC
CCTGAAGGAGGAGACCGAGGAGGAGGTGCAGGACACCAGGCTGGTGAGCGGCTGG
CGGCTGTTCAAGAAGATTAGCTAG
(pARM2384)
SEQ ID NO: 43
ATGCAGAGGTCGCCTCTGGAGAAGGCCAGCGTGGTGTCCAAGCTGTTCTTCAGCTGG
ACCAGACCAATCCTGAGGAAGGGCTACAGACAGCGCCTGGAGCTGTCAGACATCTA
CCAGATCCCTTCTGTGGACTCTGCTGACAACCTGTCTGAGAAGCTGGAGAGAGAGTG
GGACAGAGAGCTGGCCAGCAAGAAGAACCCTAAGCTGATCAACGCCCTGCGGAGGT
GCTTCTTCTGGAGATTCATGTTCTACGGAATCTTCCTGTACCTGGGGGAGGTGACCA
AGGCCGTGCAGCCTCTGCTGCTGGGAAGAATCATCGCCTCCTACGACCCCGACAAC
AAGGAGGAGCGCTCTATCGCCATCTACCTGGGCATCGGCCTGTGCCTGCTGTTCATC
GTGAGGACCCTGCTGCTGCACCCAGCCATCTTCGGCCTGCACCACATCGGAATGCAG
ATGAGAATCGCCATGTTCAGCCTGATCTACAAGAAGACCCTGAAGCTGTCAAGCAG
GGTGCTGGACAAGATCAGTATCGGACAGCTGGTGAGTCTGCTGTCCAACAACCTGA
ACAAGTTCGACGAGGGACTGGCCCTGGCCCACTTCGTGTGGATCGCTCCTCTGCAGG
TGGCCCTGCTGATGGGGCTGATCTGGGAGCTGCTGCAGGCCTCTGCCTTCTGCGGCC
TGGGCTTCCTGATCGTGCTGGCCCTGTTCCAGGCCGGCCTGGGCAGAATGATGATGA
AGTACAGAGACCAGAGAGCTGGCAAGATCAGCGAGAGACTGGTGATCACCTCAGAG
ATGATCGAGAACATCCAGTCTGTGAAGGCATACTGCTGGGAGGAGGCCATGGAGAA
GATGATCGAGAACCTGAGACAGACCGAGCTGAAGCTGACCCGGAAGGCCGCCTACG
TGAGATACTTCAACAGCAGCGCCTTCTTCTTCTCAGGGTTCTTCGTGGTGTTCCTGTC
TGTGCTGCCCTACGCCCTGATCAAGGGCATCATCCTGCGGAAGATCTTCACCACCAT
CTCATTCTGCATCGTGCTGCGCATGGCCGTGACCCGGCAGTTCCCCTGGGCCGTGCA
GACCTGGTACGACTCTCTGGGAGCCATCAACAAGATCCAGGACTTCCTGCAGAAGC
AGGAGTACAAGACCCTGGAGTACAACCTGACCACCACCGAGGTGGTGATGGAGAAC
GTGACCGCCTTCTGGGAGGAGGGATTCGGCGAGCTGTTCGAGAAGGCCAAGCAGAA
CAACAACAACAGAAAGACCTCTAACGGCGACGACAGCCTGTTCTTCAGCAACTTCA
GCCTGCTGGGCACCCCTGTGCTGAAGGACATCAACTTCAAGATCGAGAGAGGACAG
CTGCTGGCCGTGGCCGGATCCACCGGAGCCGGCAAGACCTCACTGCTGATGGTGAT
CATGGGAGAGCTGGAGCCTTCAGAGGGCAAGATCAAGCACAGTGGAAGAATCTCAT
TCTGCTCTCAGTTCTCCTGGATCATGCCTGGCACCATCAAGGAGAACATCATCTTCG
GTGTGTCCTACGACGAGTACAGATACAGAAGCGTGATCAAGGCCTGCCAGCTGGAG
GAGGACATCTCCAAGTTCGCAGAGAAGGACAACATCGTGCTGGGAGAGGGTGGCAT
CACCCTGAGCGGAGGCCAGAGGGCCAGAATCTCTCTGGCAAGAGCAGTGTACAAGG
ACGCTGACCTGTACCTGCTGGACTCTCCTTTCGGATACCTGGACGTGCTGACCGAGA
AGGAGATCTTCGAGAGCTGCGTGTGCAAGCTGATGGCCAACAAGACCAGGATCCTG
GTGACCTCTAAGATGGAGCACCTGAAGAAGGCTGACAAGATCCTGATCCTGCACGA
GGGCAGCAGCTACTTCTACGGGACCTTCAGCGAGCTGCAGAACCTGCAGCCAGACT
TCAGCAGCAAGCTGATGGGCTGCGACTCTTTCGACCAGTTCAGCGCCGAGAGAAGA
AACAGCATCCTGACCGAGACCCTGCACAGGTTCAGCCTGGAGGGCGACGCCCCTGT
GTCCTGGACCGAGACCAAGAAGCAGTCTTTCAAGCAGACCGGAGAGTTCGGCGAGA
AGAGGAAGAACTCTATCCTGAACCCAATCAACTCTATCAGGAAGTTCTCCATCGTGC
AGAAGACCCCCCTGCAGATGAACGGCATCGAGGAGGACTCTGACGAGCCTCTGGAG
AGAAGGCTGTCCCTGGTGCCAGACTCTGAGCAGGGCGAGGCCATCCTGCCTCGCAT
CAGCGTGATCAGCACCGGCCCCACCCTGCAGGCCAGGAGGAGGCAGTCTGTGCTGA
ACCTGATGACCCACAGCGTGAACCAGGGCCAGAACATCCACAGGAAGACCACCGCC
TCCACCAGGAAGGTGAGCCTGGCCCCTCAGGCCAACCTGACCGAGCTGGACATCTA
CAGCAGAAGGCTGTCTCAGGAGACCGGCCTGGAGATCAGCGAGGAGATCAACGAG
GAGGACCTGAAGGAGTGCTTCTTCGACGACATGGAGAGCATCCCAGCCGTGACCAC
CTGGAACACCTACCTGAGGTACATCACCGTGCACAAGAGCCTGATCTTCGTGCTGAT
CTGGTGCCTGGTGATCTTCCTGGCCGAGGTGGCCGCCTCTCTGGTGGTGCTGTGGCT
GCTGGGCAACACCCCTCTGCAGGACAAGGGCAACAGCACCCACAGCAGAAACAAC
AGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCAC
ACCCTGATCACCGTGAGCAAGATCCTGCACCACAAGATGCTGCACTCTGTGCTGCAG
GCCCCTATGAGCACCCTGAACACCCTGAAGGCCGGTGGGATCCTGAACAGATTCTCC
AAGGACATCGCCATCCTGGACGACCTGCTGCCTCTGACCATCTTCGACTTCATCCAG
CTGCTGCTGATCGTGATCGGAGCCATCGCCGTGGTGGCCGTGCTGCAGCCCTACATC
TTCGTGGCCACCGTGCCAGTGATCGTGGCCTTCATCATGCTGAGAGCCTACTTCCTG
CAGACCAGCCAGCAGCTGAAGCAGCTGGAGTCTGAGGGCAGGAGTCCAATCTTCAC
CCACCTGGTGACCAGCCTGAAGGGACTGTGGACCCTGAGGGCCTTCGGCCGGCAGC
CTTACTTCGAGACCCTGTTCCACAAGGCTCTGAACCTGCACACCGCCAACTGGTTCC
TGTACCTGAGCACCCTGCGCTGGTTCCAGATGAGAATCGAGATGATCTTCGTGATCT
TCTTCATCGCCGTGACCTTCATCTCCATCCTGACCACCGGCGAGGGAGAGGGAAGAG
TGGGCATCATCCTGACCCTGGCCATGAACATCATGAGCACCCTGCAGTGGGCTGTGA
ACTCCAGCATCGACGTGGACAGCCTGATGAGGTCTGTGAGCAGGGTGTTCAAGTTCA
TCGACATGCCAACCGAGGGCAAGCCTACCAAGAGCACCAAGCCATACAAGAACGGC
CAGCTGAGCAAGGTGATGATCATCGAGAACAGCCACGTGAAGAAGGACGACATCTG
GCCCAGCGGCGGCCAGATGACCGTGAAGGACCTGACCGCCAAGTACACCGAGGGCG
GCAACGCCATCCTGGAGAACATCTCCTTCTCAATCAGCCCTGGCCAGAGGGTGGGCC
TGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCT
GCAGCAGTGGAGGAAGGCCTTCGGCGTGATCCCACAGAAGGTGTTCATCTTCTCTGG
AACCTTCAGAAAGAACCTGGACCCCTACGAGCAGTGGAGCGACCAGGAGATCTGGA
AGGTGGCCGACGAGGTGGGCCTGAGATCTGTGATCGAGCAGTTCCCTGGCAAGCTG
GACTTCGTGCTGGTGGACGGGGGCTGCGTGCTGAGCCACGGCCACAAGCAGCTGAT
GTGCCTGGCCAGATCTGTGCTGAGCAAGGCCAAGATCCTGCTGCTGGACGAGCCCA
GTGCCCACCTGGACCCAGTGACCTACCAGATCATCAGAAGAACCCTGAAGCAGGCC
TTCGCCGACTGCACCGTGATCCTGTGCGAGCACAGGATCGAGGCCATGCTGGAGTG
CCAGCAGTTCCTGGTGATCGAGGAGAACAAGGTGCGGCAGTACGACTCCATCCAGA
AGCTGCTGAACGAGAGGAGCCTGTTCCGGCAGGCCATCAGCCCCTCCGACAGGGTG
AAGCTGTTCCCCCACCGGAACAGCAGCAAGTGCAAGTCTAAGCCCCAGATCGCCGC
CCTGAAGGAGGAGACCGAGGAGGAGGTGCAGGACACCAGGCTGGGCGGCGGCGGC
AGCGGCGGCAGCAGCGTGAGCAAGGGCGAGGAGCTGTTCACCGGCGTGGTGCCCAT
CCTGGTGGAGCTGGACGGCGACGTGAACGGCCACAAGTTCAGCGTGAGCGGCGAGG
GCGAGGGCGACGCCACCTACGGCAAGCTGACCCTGAAGTTCATCTGCACCACCGGC
AAGCTGCCCGTGCCCTGGCCCACCCTGGTGACCACCCTGACCTACGGCGTGCAGTGC
TTCAGCAGGTACCCCGACCACATGAAGCAGCACGACTTCTTCAAGAGCGCCATGCC
CGAGGGCTACGTGCAGGAGAGGACCATCTTCTTCAAGGACGACGGCAACTACAAGA
CCAGGGCCGAGGTGAAGTTCGAGGGCGACACCCTGGTGAACAGGATCGAGCTGAAG
GGCATCGACTTCAAGGAGGACGGCAACATCCTGGGCCACAAGCTGGAGTACAACTA
CAACAGCCACAACGTGTACATCATGGCCGACAAGCAGAAGAACGGCATCAAGGTGA
ACTTCAAGATCAGGCACAACATCGAGGACGGCAGCGTGCAGCTGGCCGACCACTAC
CAGCAGAACACCCCCATCGGCGACGGCCCCGTGCTGCTGCCCGACAACCACTACCT
GAGCACCCAGAGCGCCCTGAGCAAGGACCCCAACGAGAAGAGGGACCACATGGTG
CTGCTGGAGTTCGTGACCGCCGCCGGCATCACCCTGGGCATGGACGAGCTGTACAA
GTAG
(pARM2491)
SEQ ID NO: 44
ATGCAGAGGTCGCCTCTGGAAAAGGCCAGCGTTGTCTCCAAACTTTTTTTCAGCTGG
ACCAGACCAATTTTGAGGAAAGGATACAGACAGCGCCTGGAATTGTCAGACATATA
CCAAATCCCTTCTGTTGATTCTGCTGACAATCTATCTGAAAAATTGGAAAGAGAATG
GGATAGAGAGCTGGCTTCAAAGAAAAATCCTAAACTCATTAATGCCCTTCGGCGAT
GTTTTTTCTGGAGATTTATGTTCTATGGAATCTTTTTATATTTAGGGGAAGTCACCAA
AGCAGTACAGCCTCTCTTACTGGGAAGAATCATAGCTTCCTATGACCCGGATAACAA
GGAGGAACGCTCTATCGCGATTTATCTAGGCATAGGCTTATGCCTTCTCTTTATTGTG
AGGACACTGCTCCTACACCCAGCCATTTTTGGCCTTCATCACATTGGAATGCAGATG
AGAATAGCTATGTTTAGTTTGATTTATAAGAAGACTTTAAAGCTGTCAAGCCGTGTT
CTAGATAAAATAAGTATTGGACAACTTGTTAGTCTCCTTTCCAACAACCTGAACAAA
TTTGATGAAGGACTTGCATTGGCACATTTCGTGTGGATCGCTCCTTTGCAAGTGGCA
CTCCTCATGGGGCTAATCTGGGAGTTGTTACAGGCGTCTGCCTTCTGTGGACTTGGTT
TCCTGATAGTCCTTGCCCTTTTTCAGGCTGGGCTAGGGAGAATGATGATGAAGTACA
GAGATCAGAGAGCTGGGAAGATCAGTGAAAGACTCGTAATTACCTCAGAAATGATT
GAGAACATCCAATCTGTTAAGGCATACTGCTGGGAAGAAGCAATGGAAAAAATGAT
TGAAAACTTAAGACAAACAGAACTGAAACTGACTCGGAAGGCAGCCTATGTGAGAT
ACTTCAATAGCTCAGCCTTCTTCTTCTCAGGGTTCTTTGTGGTGTTTTTATCTGTGCTT
CCCTATGCACTAATCAAAGGAATCATCCTCCGGAAAATATTCACCACCATCTCATTC
TGCATTGTTCTGCGCATGGCGGTCACTCGGCAATTTCCCTGGGCTGTACAAACATGG
TATGACTCTCTTGGAGCAATAAACAAAATACAGGATTTCTTACAAAAGCAAGAATAT
AAGACATTGGAATATAACTTAACGACTACAGAAGTAGTGATGGAGAATGTAACAGC
CTTCTGGGAGGAGGGATTTGGGGAATTATTTGAGAAAGCAAAACAAAACAATAACA
ATAGAAAAACTTCTAATGGTGATGACAGCCTCTTCTTCAGTAATTTCTCACTTCTTGG
TACTCCTGTCCTGAAAGATATTAATTTCAAGATAGAAAGAGGACAGTTGTTGGCGGT
TGCTGGATCCACTGGAGCAGGCAAGACTTCACTTCTAATGGTGATTATGGGAGAACT
GGAGCCTTCAGAGGGTAAAATTAAGCACAGTGGAAGAATTTCATTCTGTTCTCAGTT
TTCCTGGATTATGCCTGGCACCATTAAAGAAAATATCATCTTTGGTGTTTCCTATGAT
GAATATAGATACAGAAGCGTCATCAAAGCATGCCAACTAGAAGAGGACATCTCCAA
GTTTGCAGAGAAAGACAATATAGTTCTTGGAGAAGGTGGAATCACACTGAGTGGAG
GTCAACGAGCAAGAATTTCTTTAGCAAGAGCAGTATACAAAGATGCTGATTTGTATT
TATTAGACTCTCCTTTTGGATACCTAGATGTTTTAACAGAAAAAGAAATATTTGAAA
GCTGTGTCTGTAAACTGATGGCTAACAAAACTAGGATTTTGGTCACTTCTAAAATGG
AACATTTAAAGAAAGCTGACAAAATATTAATTTTGCATGAAGGTAGCAGCTATTTTT
ATGGGACATTTTCAGAACTCCAAAATCTACAGCCAGACTTTAGCTCAAAACTCATGG
GATGTGATTCTTTCGACCAATTTAGTGCAGAAAGAAGAAATTCAATCCTAACTGAGA
CATTACACCGTTTCTCATTAGAAGGAGATGCTCCTGTCTCCTGGACAGAAACAAAAA
AACAATCTTTTAAACAGACTGGAGAGTTTGGGGAAAAAAGGAAGAATTCTATTCTC
AATCCAATCAACTCTATACGAAAATTTTCCATTGTGCAAAAGACTCCCTTACAAATG
AATGGCATCGAAGAGGATTCTGATGAGCCTTTAGAGAGAAGGCTGTCCTTAGTACC
AGATTCTGAGCAGGGAGAGGCGATACTGCCTCGCATCAGCGTGATCAGCACTGGCC
CCACGCTTCAGGCACGAAGGAGGCAGTCTGTCCTGAACCTGATGACACACTCAGTT
AACCAAGGTCAGAACATTCACCGAAAGACAACAGCATCCACACGAAAAGTGTCACT
GGCCCCTCAGGCAAACTTGACTGAACTGGATATATATTCAAGAAGGTTATCTCAAGA
AACTGGCTTGGAAATAAGTGAAGAAATTAACGAAGAAGACTTAAAGGAGTGCTTTT
TTGATGATATGGAGAGCATACCAGCAGTGACTACATGGAACACATACCTTCGATATA
TTACTGTCCACAAGAGCTTAATTTTTGTGCTAATTTGGTGCTTAGTAATTTTTCTGGC
AGAGGTGGCTGCTTCTTTGGTTGTGCTGTGGCTCCTTGGAAACACTCCTCTTCAAGAC
AAAGGGAATAGTACTCATAGTAGAAATAACAGCTATGCAGTGATTATCACCAGCAC
CAGTTCGTATTATGTGTTTTACATTTACGTGGGAGTAGCCGACACTTTGCTTGCTATG
GGATTCTTCAGAGGTCTACCACTGGTGCATACTCTAATCACAGTGTCGAAAATTTTA
CACCACAAAATGTTACATTCTGTTCTTCAAGCACCTATGTCAACCCTCAACACGTTG
AAAGCAGGTGGGATTCTTAATAGATTCTCCAAAGATATAGCAATTTTGGATGACCTT
CTGCCTCTTACCATATTTGACTTCATCCAGTTGTTATTAATTGTGATTGGAGCTATAG
CAGTTGTCGCAGTTTTACAACCCTACATCTTTGTTGCAACAGTGCCAGTGATAGTGG
CTTTTATTATGTTGAGAGCATATTTCCTCCAAACCTCACAGCAACTCAAACAACTGG
AATCTGAAGGCAGGAGTCCAATTTTCACTCATCTTGTTACAAGCTTAAAAGGACTAT
GGACACTTCGTGCCTTCGGACGGCAGCCTTACTTTGAAACTCTGTTCCACAAAGCTC
TGAATTTACATACTGCCAACTGGTTCTTGTACCTGTCAACACTGCGCTGGTTCCAAAT
GAGAATAGAAATGATTTTTGTCATCTTCTTCATTGCTGTTACCTTCATTTCCATTTTA
ACAACAGGAGAAGGAGAAGGAAGAGTTGGTATTATCCTGACTTTAGCCATGAATAT
CATGAGTACATTGCAGTGGGCTGTAAACTCCAGCATAGATGTGGATAGCTTGATGCG
ATCTGTGAGCCGAGTCTTTAAGTTCATTGACATGCCAACAGAAGGTAAACCTACCAA
GTCAACCAAACCATACAAGAATGGCCAACTCTCGAAAGTTATGATTATTGAGAATTC
ACACGTGAAGAAAGATGACATCTGGCCCTCAGGGGGCCAAATGACTGTCAAAGATC
TCACAGCAAAATACACAGAAGGTGGAAATGCCATATTAGAGAACATTTCCTTCTCA
ATAAGTCCTGGCCAGAGGGTGGGCCTCTTGGGAAGAACTGGATCAGGGAAGAGTAC
TTTGTTATCAGCTTTTTTGAGACTACTGAACACTGAAGGAGAAATCCAGATCGATGG
TGTGTCTTGGGATTCAATAACTTTGCAACAGTGGAGGAAAGCCTTTGGAGTGATACC
ACAGAAAGTATTTATTTTTTCTGGAACATTTAGAAAAAACTTGGATCCCTATGAACA
GTGGAGTGATCAAGAAATATGGAAAGTTGCAGATGAGGTTGGGCTCAGATCTGTGA
TAGAACAGTTTCCTGGGAAGCTTGACTTTGTCCTTGTGGATGGGGGCTGTGTCCTAA
GCCATGGCCACAAGCAGTTGATGTGCTTGGCTAGATCTGTTCTCAGTAAGGCGAAGA
TCTTGCTGCTTGATGAACCCAGTGCTCATTTGGATCCAGTAACATACCAAATAATTA
GAAGAACTCTAAAACAAGCATTTGCTGATTGCACAGTAATTCTCTGTGAACACAGGA
TAGAAGCAATGCTGGAATGCCAACAATTTTTGGTCATAGAAGAGAACAAAGTGCGG
CAGTACGATTCCATCCAGAAACTGCTGAACGAGAGGAGCCTCTTCCGGCAAGCCAT
CAGCCCCTCCGACAGGGTGAAGCTCTTTCCCCACCGGAACTCAAGCAAGTGCAAGT
CTAAGCCCCAGATTGCTGCTCTGAAAGAGGAGACAGAAGAAGAGGTGCAAGATACA
AGGCTTTAG
(pARM2492)
SEQ ID NO: 45
ATGCAGAGGTCGCCTCTGGAGAAGGCCAGCGTGGTGTCCAAGCTGTTCTTCAGCTGG
ACCAGACCAATCCTGAGGAAGGGCTACAGACAGCGCCTGGAGCTGTCAGACATCTA
CCAGATCCCTTCTGTGGACTCTGCTGACAACCTGTCTGAGAAGCTGGAGAGAGAGTG
GGACAGAGAGCTGGCCAGCAAGAAGAACCCTAAGCTGATCAACGCCCTGCGGAGGT
GCTTCTTCTGGAGATTCATGTTCTACGGAATCTTCCTGTACCTGGGGGAGGTGACCA
AGGCCGTGCAGCCTCTGCTGCTGGGAAGAATCATCGCCTCCTACGACCCCGACAAC
AAGGAGGAGCGCTCTATCGCCATCTACCTGGGCATCGGCCTGTGCCTGCTGTTCATC
GTGAGGACCCTGCTGCTGCACCCAGCCATCTTCGGCCTGCACCACATCGGAATGCAG
ATGAGAATCGCCATGTTCAGCCTGATCTACAAGAAGACCCTGAAGCTGTCAAGCAG
GGTGCTGGACAAGATCAGTATCGGACAGCTGGTGAGTCTGCTGTCCAACAACCTGA
ACAAGTTCGACGAGGGACTGGCCCTGGCCCACTTCGTGTGGATCGCTCCTCTGCAGG
TGGCCCTGCTGATGGGGCTGATCTGGGAGCTGCTGCAGGCCTCTGCCTTCTGCGGCC
TGGGCTTCCTGATCGTGCTGGCCCTGTTCCAGGCCGGCCTGGGCAGAATGATGATGA
AGTACAGAGACCAGAGAGCTGGCAAGATCAGCGAGAGACTGGTGATCACCTCAGAG
ATGATCGAGAACATCCAGTCTGTGAAGGCATACTGCTGGGAGGAGGCCATGGAGAA
GATGATCGAGAACCTGAGACAGACCGAGCTGAAGCTGACCCGGAAGGCCGCCTACG
TGAGATACTTCAACAGCAGCGCCTTCTTCTTCTCAGGGTTCTTCGTGGTGTTCCTGTC
TGTGCTGCCCTACGCCCTGATCAAGGGCATCATCCTGCGGAAGATCTTCACCACCAT
CTCATTCTGCATCGTGCTGCGCATGGCCGTGACCCGGCAGTTCCCCTGGGCCGTGCA
GACCTGGTACGACTCTCTGGGAGCCATCAACAAGATCCAGGACTTCCTGCAGAAGC
AGGAGTACAAGACCCTGGAGTACAACCTGACCACCACCGAGGTGGTGATGGAGAAC
GTGACCGCCTTCTGGGAGGAGGGATTCGGCGAGCTGTTCGAGAAGGCCAAGCAGAA
CAACAACAACAGAAAGACCTCTAACGGCGACGACAGCCTGTTCTTCAGCAACTTCA
GCCTGCTGGGCACCCCTGTGCTGAAGGACATCAACTTCAAGATCGAGAGAGGACAG
CTGCTGGCCGTGGCCGGATCCACCGGAGCCGGCAAGACCTCACTGCTGATGGTGAT
CATGGGAGAGCTGGAGCCTTCAGAGGGCAAGATCAAGCACAGTGGAAGAATCTCAT
TCTGCTCTCAGTTCTCCTGGATCATGCCTGGCACCATCAAGGAGAACATCATCTTCG
GTGTGTCCTACGACGAGTACAGATACAGAAGCGTGATCAAGGCCTGCCAGCTGGAG
GAGGACATCTCCAAGTTCGCAGAGAAGGACAACATCGTGCTGGGAGAGGGTGGCAT
CACCCTGAGCGGAGGCCAGAGGGCCAGAATCTCTCTGGCAAGAGCAGTGTACAAGG
ACGCTGACCTGTACCTGCTGGACTCTCCTTTCGGATACCTGGACGTGCTGACCGAGA
AGGAGATCTTCGAGAGCTGCGTGTGCAAGCTGATGGCCAACAAGACCAGGATCCTG
GTGACCTCTAAGATGGAGCACCTGAAGAAGGCTGACAAGATCCTGATCCTGCACGA
GGGCAGCAGCTACTTCTACGGGACCTTCAGCGAGCTGCAGAACCTGCAGCCAGACT
TCAGCAGCAAGCTGATGGGCTGCGACTCTTTCGACCAGTTCAGCGCCGAGAGAAGA
AACAGCATCCTGACCGAGACCCTGCACAGGTTCAGCCTGGAGGGCGACGCCCCTGT
GTCCTGGACCGAGACCAAGAAGCAGTCTTTCAAGCAGACCGGAGAGTTCGGCGAGA
AGAGGAAGAACTCTATCCTGAACCCAATCAACTCTATCAGGAAGTTCTCCATCGTGC
AGAAGACCCCCCTGCAGATGAACGGCATCGAGGAGGACTCTGACGAGCCTCTGGAG
AGAAGGCTGTCCCTGGTGCCAGACTCTGAGCAGGGCGAGGCCATCCTGCCTCGCAT
CAGCGTGATCAGCACCGGCCCCACCCTGCAGGCCAGGAGGAGGCAGTCTGTGCTGA
ACCTGATGACCCACAGCGTGAACCAGGGCCAGAACATCCACAGGAAGACCACCGCC
TCCACCAGGAAGGTGAGCCTGGCCCCTCAGGCCAACCTGACCGAGCTGGACATCTA
CAGCAGAAGGCTGTCTCAGGAGACCGGCCTGGAGATCAGCGAGGAGATCAACGAG
GAGGACCTGAAGGAGTGCTTCTTCGACGACATGGAGAGCATCCCAGCCGTGACCAC
CTGGAACACCTACCTGAGGTACATCACCGTGCACAAGAGCCTGATCTTCGTGCTGAT
CTGGTGCCTGGTGATCTTCCTGGCCGAGGTGGCCGCCTCTCTGGTGGTGCTGTGGCT
GCTGGGCAACACCCCTCTGCAGGACAAGGGCAACAGCACCCACAGCAGAAACAAC
AGCTACGCCGTGATCATCACCAGCACCAGCAGCTACTACGTGTTCTACATCTACGTG
GGAGTGGCCGACACCCTGCTGGCCATGGGCTTCTTCAGAGGTCTGCCACTGGTGCAC
ACCCTGATCACCGTGAGCAAGATCCTGCACCACAAGATGCTGCACTCTGTGCTGCAG
GCCCCTATGAGCACCCTGAACACCCTGAAGGCCGGTGGGATCCTGAACAGATTCTCC
AAGGACATCGCCATCCTGGACGACCTGCTGCCTCTGACCATCTTCGACTTCATCCAG
CTGCTGCTGATCGTGATCGGAGCCATCGCCGTGGTGGCCGTGCTGCAGCCCTACATC
TTCGTGGCCACCGTGCCAGTGATCGTGGCCTTCATCATGCTGAGAGCCTACTTCCTG
CAGACCAGCCAGCAGCTGAAGCAGCTGGAGTCTGAGGGCAGGAGTCCAATCTTCAC
CCACCTGGTGACCAGCCTGAAGGGACTGTGGACCCTGAGGGCCTTCGGCCGGCAGC
CTTACTTCGAGACCCTGTTCCACAAGGCTCTGAACCTGCACACCGCCAACTGGTTCC
TGTACCTGAGCACCCTGCGCTGGTTCCAGATGAGAATCGAGATGATCTTCGTGATCT
TCTTCATCGCCGTGACCTTCATCTCCATCCTGACCACCGGCGAGGGAGAGGGAAGAG
TGGGCATCATCCTGACCCTGGCCATGAACATCATGAGCACCCTGCAGTGGGCTGTGA
ACTCCAGCATCGACGTGGACAGCCTGATGAGGTCTGTGAGCAGGGTGTTCAAGTTCA
TCGACATGCCAACCGAGGGCAAGCCTACCAAGAGCACCAAGCCATACAAGAACGGC
CAGCTGAGCAAGGTGATGATCATCGAGAACAGCCACGTGAAGAAGGACGACATCTG
GCCCAGCGGCGGCCAGATGACCGTGAAGGACCTGACCGCCAAGTACACCGAGGGCG
GCAACGCCATCCTGGAGAACATCTCCTTCTCAATCAGCCCTGGCCAGAGGGTGGGCC
TGCTGGGAAGAACCGGCTCAGGCAAGAGCACCCTGCTGAGCGCCTTCCTGAGACTG
CTGAACACCGAGGGCGAGATCCAGATCGACGGCGTGTCTTGGGACTCAATCACCCT
GCAGCAGTGGAGGAAGGCCTTCGGCGTGATCCCACAGAAGGTGTTCATCTTCTCTGG
AACCTTCAGAAAGAACCTGGACCCCTACGAGCAGTGGAGCGACCAGGAGATCTGGA
AGGTGGCCGACGAGGTGGGCCTGAGATCTGTGATCGAGCAGTTCCCTGGCAAGCTG
GACTTCGTGCTGGTGGACGGGGGCTGCGTGCTGAGCCACGGCCACAAGCAGCTGAT
GTGCCTGGCCAGATCTGTGCTGAGCAAGGCCAAGATCCTGCTGCTGGACGAGCCCA
GTGCCCACCTGGACCCAGTGACCTACCAGATCATCAGAAGAACCCTGAAGCAGGCC
TTCGCCGACTGCACCGTGATCCTGTGCGAGCACAGGATCGAGGCCATGCTGGAGTG
CCAGCAGTTCCTGGTGATCGAGGAGAACAAGGTGCGGCAGTACGACTCCATCCAGA
AGCTGCTGAACGAGAGGAGCCTGTTCCGGCAGGCCATCAGCCCCTCCGACAGGGTG
AAGCTGTTCCCCCACCGGAACAGCAGCAAGTGCAAGTCTAAGCCCCAGATCGCCGC
CCTGAAGGAGGAGACCGAGGAGGAGGTGCAGGACACCAGGCTGTAG
(pARM2493)
SEQ ID NO: 46
ATGCAGAGGAGCCCCCTGGAGAAGGCTAGCGTGGTGAGCAAGCTGTTCTTCAGCTG
GACCAGACCAATCCTGAGGAAGGGCTACAGACAGCGCCTGGAGCTGAGCGACATCT
ACCAGATCCCCAGCGTGGACAGCGCCGACAACCTGAGCGAGAAGCTGGAGAGAGA
GTGGGACAGAGAGCTGGCCAGCAAGAAGAACCCCAAGCTGATCAACGCCCTGCGG
AGGTGCTTCTTCTGGAGATTCATGTTCTACGGAATCTTCCTGTACCTGGGGGAGGTG
ACCAAGGCCGTGCAGCCCCTGCTGCTGGGAAGAATCATCGCCAGCTACGACCCCGA
CAACAAGGAGGAGCGCAGCATCGCCATCTACCTGGGCATCGGCCTGTGCCTGCTGTT
CATCGTGAGGACCCTGCTGCTGCACCCAGCCATCTTCGGCCTGCACCACATCGGAAT
GCAGATGAGAATCGCCATGTTCAGCCTGATCTACAAGAAGACCCTGAAGCTGAGCA
GCAGGGTGCTGGACAAGATCAGCATCGGACAGCTGGTGAGCCTGCTGAGCAACAAC
CTGAACAAGTTCGACGAGGGACTGGCCCTGGCCCACTTCGTGTGGATCGCCCCACTG
CAGGTGGCCCTGCTGATGGGGCTGATCTGGGAGCTGCTGCAGGCCAGCGCCTTCTGC
GGCCTGGGCTTCCTGATCGTGCTGGCCCTGTTCCAGGCCGGCCTGGGCAGAATGATG
ATGAAGTACAGAGACCAGAGAGCCGGCAAGATCAGCGAGAGACTGGTGATCACCA
GCGAGATGATCGAGAACATCCAGAGCGTGAAGGCATACTGCTGGGAGGAGGCCATG
GAGAAGATGATCGAGAACCTGAGACAGACCGAGCTGAAGCTGACCCGGAAGGCCG
CCTACGTGAGATACTTCAACAGCAGCGCCTTCTTCTTCAGCGGGTTCTTCGTGGTGTT
CCTGAGCGTGCTGCCCTACGCCCTGATCAAGGGCATCATCCTGCGGAAGATCTTCAC
CACCATCAGCTTCTGCATCGTGCTGCGCATGGCCGTGACCCGGCAGTTCCCCTGGGC
CGTGCAGACCTGGTACGACAGCCTGGGAGCCATCAACAAGATCCAGGACTTCCTGC
AGAAGCAGGAGTACAAGACCCTGGAGTACAACCTGACCACCACCGAGGTGGTGATG
GAGAACGTGACCGCCTTCTGGGAGGAGGGATTCGGCGAGCTGTTCGAGAAGGCCAA
GCAGAACAACAACAACAGAAAGACCAGCAACGGCGACGACAGCCTGTTCTTCAGCA
ACTTCAGCCTGCTGGGCACCCCCGTGCTGAAGGACATCAACTTCAAGATCGAGAGA
GGACAGCTGCTGGCCGTGGCCGGAAGCACCGGAGCCGGCAAGACCAGCCTGCTGAT
GGTGATCATGGGAGAGCTGGAGCCCAGCGAGGGCAAGATCAAGCACAGCGGAAGA
ATCAGCTTCTGCAGCCAGTTCAGCTGGATCATGCCCGGCACCATCAAGGAGAACATC
ATCTTCGGCGTGAGCTACGACGAGTACAGATACAGAAGCGTGATCAAGGCCTGCCA
GCTGGAGGAGGACATCAGCAAGTTCGCAGAGAAGGACAACATCGTGCTGGGAGAG
GGCGGCATCACCCTGAGCGGAGGCCAGAGGGCCAGAATCAGCCTGGCAAGAGCAG
TGTACAAGGACGCCGACCTGTACCTGCTGGACAGCCCCTTCGGATACCTGGACGTGC
TGACCGAGAAGGAGATCTTCGAGAGCTGCGTGTGCAAGCTGATGGCCAACAAGACC
AGGATCCTGGTGACCAGCAAGATGGAGCACCTGAAGAAGGCCGACAAGATCCTGAT
CCTGCACGAGGGCAGCAGCTACTTCTACGGGACCTTCAGCGAGCTGCAGAACCTGC
AGCCAGACTTCAGCAGCAAGCTGATGGGCTGCGACAGCTTCGACCAGTTCAGCGCC
GAGAGAAGAAACAGCATCCTGACCGAGACCCTGCACAGGTTCAGCCTGGAGGGCGA
CGCCCCCGTGAGCTGGACCGAGACCAAGAAGCAGAGCTTCAAGCAGACCGGAGAGT
TCGGCGAGAAGAGGAAGAACAGCATCCTGAACCCAATCAACAGCATCAGGAAGTTC
AGCATCGTGCAGAAGACCCCACTGCAGATGAACGGCATCGAGGAGGACAGCGACG
AGCCCCTGGAGAGAAGGCTGAGCCTGGTGCCAGACAGCGAGCAGGGCGAGGCCAT
CCTGCCCCGCATCAGCGTGATCAGCACCGGCCCCACCCTGCAGGCCAGGAGGAGGC
AGAGCGTGCTGAACCTGATGACCCACAGCGTGAACCAGGGCCAGAACATCCACAGG
AAGACCACCGCCAGCACCAGGAAGGTGAGCCTGGCCCCACAGGCCAACCTGACCGA
GCTGGACATCTACAGCAGAAGGCTGAGCCAGGAGACCGGCCTGGAGATCAGCGAG
GAGATCAACGAGGAGGACCTGAAGGAGTGCTTCTTCGACGACATGGAGAGCATCCC
AGCCGTGACCACCTGGAACACCTACCTGAGGTACATCACCGTGCACAAGAGCCTGA
TCTTCGTGCTGATCTGGTGCCTGGTGATCTTCCTGGCCGAGGTGGCCGCCAGCCTGG
TGGTGCTGTGGCTGCTGGGCAACACCCCACTGCAGGACAAGGGCAACAGCACCCAC
AGCAGAAACAACAGCTACGCCGTGATCATCACCAGCACCAGCAGCTACTACGTGTT
CTACATCTACGTGGGAGTGGCCGACACCCTGCTGGCCATGGGCTTCTTCAGAGGCCT
GCCACTGGTGCACACCCTGATCACCGTGAGCAAGATCCTGCACCACAAGATGCTGC
ACAGCGTGCTGCAGGCCCCCATGAGCACCCTGAACACCCTGAAGGCCGGCGGGATC
CTGAACAGATTCAGCAAGGACATCGCCATCCTGGACGACCTGCTGCCCCTGACCATC
TTCGACTTCATCCAGCTGCTGCTGATCGTGATCGGAGCCATCGCCGTGGTGGCCGTG
CTGCAGCCCTACATCTTCGTGGCCACCGTGCCAGTGATCGTGGCCTTCATCATGCTG
AGAGCCTACTTCCTGCAGACCAGCCAGCAGCTGAAGCAGCTGGAGAGCGAGGGCAG
GAGCCCAATCTTCACCCACCTGGTGACCAGCCTGAAGGGACTGTGGACCCTGAGGG
CCTTCGGCCGGCAGCCCTACTTCGAGACCCTGTTCCACAAGGCCCTGAACCTGCACA
CCGCCAACTGGTTCCTGTACCTGAGCACCCTGCGCTGGTTCCAGATGAGAATCGAGA
TGATCTTCGTGATCTTCTTCATCGCCGTGACCTTCATCTCCATCCTGACCACCGGCGA
GGGAGAGGGAAGAGTGGGCATCATCCTGACCCTGGCCATGAACATCATGAGCACCC
TGCAGTGGGCTGTGAACTCCAGCATCGACGTGGACAGCCTGATGAGGTCTGTGAGC
AGGGTGTTCAAGTTCATCGACATGCCAACCGAGGGCAAGCCTACCAAGAGCACCAA
GCCATACAAGAACGGCCAGCTGAGCAAGGTGATGATCATCGAGAACAGCCACGTGA
AGAAGGACGACATCTGGCCCAGCGGCGGCCAGATGACCGTGAAGGACCTGACCGCC
AAGTACACCGAGGGCGGCAACGCCATCCTGGAGAACATCTCCTTCTCAATCAGCCCT
GGCCAGAGGGTGGGCCTGCTGGGAAGAACCGGCAGCGGCAAGAGCACCCTGCTGA
GCGCCTTCCTGAGACTGCTGAACACCGAGGGCGAGATCCAGATCGACGGCGTGTCT
TGGGACTCAATCACCCTGCAGCAGTGGAGGAAGGCCTTCGGCGTGATCCCACAGAA
GGTGTTCATCTTCTCTGGAACCTTCAGAAAGAACCTGGACCCCTACGAGCAGTGGAG
CGACCAGGAGATCTGGAAGGTGGCCGACGAGGTGGGCCTGAGATCTGTGATCGAGC
AGTTCCCTGGCAAGCTGGACTTCGTGCTGGTGGACGGGGGCTGCGTGCTGAGCCACG
GCCACAAGCAGCTGATGTGCCTGGCCAGATCTGTGCTGAGCAAGGCCAAGATCCTG
CTGCTGGACGAGCCCAGTGCCCACCTGGACCCAGTGACCTACCAGATCATCAGAAG
AACCCTGAAGCAGGCCTTCGCCGACTGCACCGTGATCCTGTGCGAGCACAGGATCG
AGGCCATGCTGGAGTGCCAGCAGTTCCTGGTGATCGAGGAGAACAAGGTGCGGCAG
TACGACTCCATCCAGAAGCTGCTGAACGAGAGGAGCCTGTTCCGGCAGGCCATCAG
CCCCTCCGACAGGGTGAAGCTGTTCCCCCACCGGAACAGCAGCAAGTGCAAGTCTA
AGCCCCAGATCGCCGCCCTGAAGGAGGAGACCGAGGAGGAGGTGCAGGACACCAG
GCTGTAG

(mARM764)
SEQ ID NO: 47
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAAAAGGCCAGCGUUGUCUCCAAACUUUUUUUCAGCUGGACCAG
ACCAAUUUUGAGGAAAGGAUACAGACAGCGCCUGGAAUUGUCAGACAUA
UACCAAAUCCCUUCUGUUGAUUCUGCUGACAAUCUAUCUGAAAAAUUGG
AAAGAGAAUGGGAUAGAGAGCUGGCUUCAAAGAAAAAUCCUAAACUCAU
UAAUGCCCUUCGGCGAUGUUUUUUCUGGAGAUUUAUGUUCUAUGGAAUC
UUUUUAUAUUUAGGGGAAGUCACCAAAGCAGUACAGCCUCUCUUACUGG
GAAGAAUCAUAGCUUCCUAUGACCCGGAUAACAAGGAGGAACGCUCUAU
CGCGAUUUAUCUAGGCAUAGGCUUAUGCCUUCUCUUUAUUGUGAGGACA
CUGCUCCUACACCCAGCCAUUUUUGGCCUUCAUCACAUUGGAAUGCAGA
UGAGAAUAGCUAUGUUUAGUUUGAUUUAUAAGAAGACUUUAAAGCUGUC
AAGCCGUGUUCUAGAUAAAAUAAGUAUUGGACAACUUGUUAGUCUCCUU
UCCAACAACCUGAACAAAUUUGAUGAAGGACUUGCAUUGGCACAUUUCG
UGUGGAUCGCUCCUUUGCAAGUGGCACUCCUCAUGGGGCUAAUCUGGGA
GUUGUUACAGGCGUCUGCCUUCUGUGGACUUGGUUUCCUGAUAGUCCUU
GCCCUUUUUCAGGCUGGGCUAGGGAGAAUGAUGAUGAAGUACAGAGAUC
AGAGAGCUGGGAAGAUCAGUGAAAGACUCGUAAUUACCUCAGAAAUGAU
UGAGAACAUCCAAUCUGUUAAGGCAUACUGCUGGGAAGAAGCAAUGGAA
AAAAUGAUUGAAAACUUAAGACAAACAGAACUGAAACUGACUCGGAAGG
CAGCCUAUGUGAGAUACUUCAAUAGCUCAGCCUUCUUCUUCUCAGGGUU
CUUUGUGGUGUUUUUAUCUGUGCUUCCCUAUGCACUAAUCAAAGGAAUC
AUCCUCCGGAAAAUAUUCACCACCAUCUCAUUCUGCAUUGUUCUGCGCA
UGGCGGUCACUCGGCAAUUUCCCUGGGCUGUACAAACAUGGUAUGACUC
UCUUGGAGCAAUAAACAAAAUACAGGAUUUCUUACAAAAGCAAGAAUAU
AAGACAUUGGAAUAUAACUUAACGACUACAGAAGUAGUGAUGGAGAAUG
UAACAGCCUUCUGGGAGGAGGGAUUUGGGGAAUUAUUUGAGAAAGCAAA
ACAAAACAAUAACAAUAGAAAAACUUCUAAUGGUGAUGACAGCCUCUUC
UUCAGUAAUUUCUCACUUCUUGGUACUCCUGUCCUGAAAGAUAUUAAUU
UCAAGAUAGAAAGAGGACAGUUGUUGGCGGUUGCUGGAUCCACUGGAGC
AGGCAAGACUUCACUUCUAAUGGUGAUUAUGGGAGAACUGGAGCCUUCA
GAGGGUAAAAUUAAGCACAGUGGAAGAAUUUCAUUCUGUUCUCAGUUUU
CCUGGAUUAUGCCUGGCACCAUUAAAGAAAAUAUCAUCUUUGGUGUUUC
CUAUGAUGAAUAUAGAUACAGAAGCGUCAUCAAAGCAUGCCAACUAGAA
GAGGACAUCUCCAAGUUUGCAGAGAAAGACAAUAUAGUUCUUGGAGAAG
GUGGAAUCACACUGAGUGGAGGUCAACGAGCAAGAAUUUCUUUAGCAAG
AGCAGUAUACAAAGAUGCUGAUUUGUAUUUAUUAGACUCUCCUUUUGGA
UACCUAGAUGUUUUAACAGAAAAAGAAAUAUUUGAAAGCUGUGUCUGUA
AACUGAUGGCUAACAAAACUAGGAUUUUGGUCACUUCUAAAAUGGAACA
UUUAAAGAAAGCUGACAAAAUAUUAAUUUUGCAUGAAGGUAGCAGCUAU
UUUUAUGGGACAUUUUCAGAACUCCAAAAUCUACAGCCAGACUUUAGCU
CAAAACUCAUGGGAUGUGAUUCUUUCGACCAAUUUAGUGCAGAAAGAAG
AAAUUCAAUCCUAACUGAGACAUUACACCGUUUCUCAUUAGAAGGAGAU
GCUCCUGUCUCCUGGACAGAAACAAAAAAACAAUCUUUUAAACAGACUG
GAGAGUUUGGGGAAAAAAGGAAGAAUUCUAUUCUCAAUCCAAUCAACUC
UAUACGAAAAUUUUCCAUUGUGCAAAAGACUCCCUUACAAAUGAAUGGC
AUCGAAGAGGAUUCUGAUGAGCCUUUAGAGAGAAGGCUGUCCUUAGUAC
CAGAUUCUGAGCAGGGAGAGGCGAUACUGCCUCGCAUCAGCGUGAUCAG
CACUGGCCCCACGCUUCAGGCACGAAGGAGGCAGUCUGUCCUGAACCUG
AUGACACACUCAGUUAACCAAGGUCAGAACAUUCACCGAAAGACAACAG
CAUCCACACGAAAAGUGUCACUGGCCCCUCAGGCAAACUUGACUGAACU
GGAUAUAUAUUCAAGAAGGUUAUCUCAAGAAACUGGCUUGGAAAUAAGU
GAAGAAAUUAACGAAGAAGACUUAAAGGAGUGCUUUUUUGAUGAUAUGG
AGAGCAUACCAGCAGUGACUACAUGGAACACAUACCUUCGAUAUAUUAC
UGUCCACAAGAGCUUAAUUUUUGUGCUAAUUUGGUGCUUAGUAAUUUUU
CUGGCAGAGGUGGCUGCUUCUUUGGUUGUGCUGUGGCUCCUUGGAAACA
CUCCUCUUCAAGACAAAGGGAAUAGUACUCAUAGUAGAAAUAACAGCUA
UGCAGUGAUUAUCACCAGCACCAGUUCGUAUUAUGUGUUUUACAUUUAC
GUGGGAGUAGCCGACACUUUGCUUGCUAUGGGAUUCUUCAGAGGUCUAC
CACUGGUGCAUACUCUAAUCACAGUGUCGAAAAUUUUACACCACAAAAU
GUUACAUUCUGUUCUUCAAGCACCUAUGUCAACCCUCAACACGUUGAAA
GCAGGUGGGAUUCUUAAUAGAUUCUCCAAAGAUAUAGCAAUUUUGGAUG
ACCUUCUGCCUCUUACCAUAUUUGACUUCAUCCAGUUGUUAUUAAUUGU
GAUUGGAGCUAUAGCAGUUGUCGCAGUUUUACAACCCUACAUCUUUGUU
GCAACAGUGCCAGUGAUAGUGGCUUUUAUUAUGUUGAGAGCAUAUUUCC
UCCAAACCUCACAGCAACUCAAACAACUGGAAUCUGAAGGCAGGAGUCC
AAUUUUCACUCAUCUUGUUACAAGCUUAAAAGGACUAUGGACACUUCGU
GCCUUCGGACGGCAGCCUUACUUUGAAACUCUGUUCCACAAAGCUCUGA
AUUUACAUACUGCCAACUGGUUCUUGUACCUGUCAACACUGCGCUGGUU
CCAAAUGAGAAUAGAAAUGAUUUUUGUCAUCUUCUUCAUUGCUGUUACC
UUCAUUUCCAUUUUAACAACAGGAGAAGGAGAAGGAAGAGUUGGUAUUA
UCCUGACUUUAGCCAUGAAUAUCAUGAGUACAUUGCAGUGGGCUGUAAA
CUCCAGCAUAGAUGUGGAUAGCUUGAUGCGAUCUGUGAGCCGAGUCUUU
AAGUUCAUUGACAUGCCAACAGAAGGUAAACCUACCAAGUCAACCAAAC
CAUACAAGAAUGGCCAACUCUCGAAAGUUAUGAUUAUUGAGAAUUCACA
CGUGAAGAAAGAUGACAUCUGGCCCUCAGGGGGCCAAAUGACUGUCAAA
GAUCUCACAGCAAAAUACACAGAAGGUGGAAAUGCCAUAUUAGAGAACA
UUUCCUUCUCAAUAAGUCCUGGCCAGAGGGUGGGCCUCUUGGGAAGAAC
UGGAUCAGGGAAGAGUACUUUGUUAUCAGCUUUUUUGAGACUACUGAAC
ACUGAAGGAGAAAUCCAGAUCGAUGGUGUGUCUUGGGAUUCAAUAACUU
UGCAACAGUGGAGGAAAGCCUUUGGAGUGAUACCACAGAAAGUAUUUAU
UUUUUCUGGAACAUUUAGAAAAAACUUGGAUCCCUAUGAACAGUGGAGU
GAUCAAGAAAUAUGGAAAGUUGCAGAUGAGGUUGGGCUCAGAUCUGUGA
UAGAACAGUUUCCUGGGAAGCUUGACUUUGUCCUUGUGGAUGGGGGCUG
UGUCCUAAGCCAUGGCCACAAGCAGUUGAUGUGCUUGGCUAGAUCUGUU
CUCAGUAAGGCGAAGAUCUUGCUGCUUGAUGAACCCAGUGCUCAUUUGG
AUCCAGUAACAUACCAAAUAAUUAGAAGAACUCUAAAACAAGCAUUUGC
UGAUUGCACAGUAAUUCUCUGUGAACACAGGAUAGAAGCAAUGCUGGAA
UGCCAACAAUUUUUGGUCAUAGAAGAGAACAAAGUGCGGCAGUACGAUU
CCAUCCAGAAACUGCUGAACGAGAGGAGCCUCUUCCGGCAAGCCAUCAG
CCCCUCCGACAGGGUGAAGCUCUUUCCCCACCGGAACUCAAGCAAGUGC
AAGUCUAAGCCCCAGAUUGCUGCUCUGAAAGAGGAGACAGAAGAAGAGG
UGCAAGAUACAAGGCUUUAGCUCGAGCUAGUGACUGACUAGGAUCUGGU
UACCACUAAACCAGCCUCAAGAACACCCGAAUGGAGUCUCUAAGCUACA
UAAUACCAACUUACACUUACAAAAUGUUGUCCCCCAAAAUGUAGCCAUU
CGUAUCUGCUCCUAAUAAAAAGAAAGUUUCUUCACAUUCUAG
(mARM766)
SEQ ID NO: 48
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUUGUCUCCAAGCUGUUCUUCAGCUGGACCAG
ACCAAUUUUGAGGAAAGGAUACAGACAGCGCCUGGAAUUGUCAGACAUA
UACCAAAUCCCUUCUGUUGAUUCUGCUGACAAUCUAUCUGAGAAGUUGG
AAAGAGAAUGGGAUAGAGAGCUGGCUUCCAAGAAGAACCCUAAGCUCAU
UAAUGCCCUUCGGCGAUGCUUUUUCUGGAGGUUCAUGUUCUAUGGAAUC
UUCCUGUACUUAGGGGAGGUCACCAAGGCAGUACAGCCUCUCUUGCUGG
GCAGAAUCAUAGCUUCCUAUGACCCUGAUAACAAGGAGGAACGCAGCAU
CGCGAUCUACCUGGGCAUCGGCUUGUGCCUGCUCUUUAUCGUGAGGACA
CUGCUCCUACACCCUGCCAUCUUUGGCCUUCAUCACAUUGGAAUGCAGA
UGAGAAUCGCUAUGUUCAGUUUGAUUUACAAGAAGACUUUAAAGCUGUC
CAGCAGGGUGCUAGAUAAGAUCAGCAUUGGACAGCUUGUUAGCCUGCUU
UCCAACAACCUGAACAAGUUCGAUGAAGGACUGGCAUUGGCACAUUUCG
UGUGGAUCGCUCCUCUGCAAGUGGCACUCCUGAUGGGGUUGAUCUGGGA
GUUGCUGCAGGCGAGCGCCUUCUGUGGACUUGGCUUCCUGAUAGUCCUU
GCCCUGUUCCAGGCUGGGCUAGGGAGAAUGAUGAUGAAGUACAGAGAUC
AGAGGGCUGGGAAGAUCAGCGAGAGACUCGUGAUCACCUCUGAGAUGAU
CGAGAACAUCCAGUCUGUGAAGGCAUACUGCUGGGAAGAGGCAAUGGAG
AAGAUGAUUGAGAACUUAAGACAGACAGAGCUGAAGCUGACUCGGAAGG
CAGCCUAUGUGAGAUACUUCAACAGCUCAGCCUUCUUCUUCAGCGGGUU
CUUUGUGGUCUUCCUGUCUGUGCUUCCCUAUGCACUAAUCAAGGGAAUC
AUUCUGCGGAAGAUCUUCACAACCAUCUCCUUCUGCAUUGUGCUGCGCA
UGGCGGUCACUCGGCAGUUUCCCUGGGCUGUACAGACAUGGUAUGACUC
UCUGGGAGCCAUCAACAAGAUACAGGAUUUCCUGCAGAAGCAAGAGUAU
AAGACAUUGGAGUACAACUUAACGACUACAGAAGUAGUGAUGGAGAACG
UAACCGCCUUCUGGGAGGAGGGAUUUGGGGAGUUGUUCGAGAAAGCAAA
GCAGAACAACAAUAAUCGGAAGACCUCCAAUGGUGAUGACAGCCUCUUC
UUCAGUAACUUCAGCCUUCUUGGUACUCCUGUCCUGAAGGACAUCAACU
UCAAGAUAGAGAGGGGACAGUUGUUGGCGGUUGCUGGAUCCACUGGAGC
AGGCAAGACUUCACUUCUAAUGGUGAUCAUGGGAGAACUGGAGCCUAGC
GAGGGCAAGAUCAAGCACAGUGGAAGGAUCUCAUUCUGUUCUCAGUUUU
CCUGGAUUAUGCCUGGCACCAUUAAGGAGAACAUCAUCUUUGGUGUUUC
CUAUGAUGAGUACCGCUACAGAAGCGUCAUCAAGGCAUGCCAACUAGAA
GAGGACAUCUCCAAGUUUGCAGAGAAGGACAAUAUAGUUCUUGGAGAAG
GUGGAAUCACACUGAGUGGAGGUCAACGAGCAAGAAUCUCUUUAGCAAG
AGCAGUAUACAAGGACGCUGAUUUGUACUUGUUAGACUCUCCCUUUGGA
UACCUAGAUGUGCUGACCGAGAAGGAGAUAUUCGAAAGCUGUGUCUGUA
AGCUGAUGGCUAACAAGACUAGGAUCUUGGUCACUUCUAAGAUGGAACA
CCUGAAGAAAGCUGACAAGAUCUUGAUCCUGCAUGAAGGUUCUAGCUAC
UUCUACGGGACAUUUUCAGAACUCCAGAAUCUACAGCCAGACUUUAGCU
CAAAGCUCAUGGGAUGUGAUUCUUUCGACCAGUUUAGUGCAGAGAGACG
GAACUCAAUCCUAACUGAGACAUUACACCGUUUCUCAUUAGAAGGAGAU
GCUCCUGUCUCCUGGACAGAGACGAAGAAACAGUCUUUUAAACAGACUG
GAGAGUUUGGGGAGAAACGCAAGAACAGCAUUCUCAAUCCAAUCAACUC
UAUACGAAAGUUCUCCAUUGUGCAGAAGACUCCCUUACAGAUGAAUGGC
AUCGAAGAGGAUUCUGAUGAGCCUUUAGAGAGAAGGCUGUCCUUAGUAC
CAGAUUCUGAGCAGGGAGAGGCGAUACUGCCUCGCAUCAGCGUGAUCAG
CACUGGCCCCACGCUUCAGGCACGAAGGCGCCAGUCUGUCCUGAACCUG
AUGACACACUCAGUUAACCAAGGUCAGAACAUUCACCGAAAGACAACCG
CAUCCACAAGGAAGGUGUCACUGGCCCCUCAGGCAAACUUGACUGAACU
GGACAUCUACUCCAGAAGGUUAUCUCAGGAGACUGGCUUGGAGAUCAGU
GAAGAGAUUAACGAAGAGGACUUAAAGGAGUGCUUCUUUGAUGAUAUGG
AGAGCAUACCAGCAGUGACUACAUGGAACACAUACCUUAGGUACAUCAC
UGUCCACAAGAGCCUGAUCUUCGUGCUAAUUUGGUGCUUGGUGAUCUUC
CUGGCAGAGGUGGCUGCUUCUUUGGUUGUGCUGUGGCUCCUUGGAAACA
CUCCUCUUCAAGACAAAGGGAAUAGUACUCAUUCCAGCAACAAUUCCUA
UGCAGUGAUUAUCACCAGCACCAGUUCGUAUUAUGUGUUCUACAUUUAC
GUGGGAGUAGCCGACACUUUGCUUGCUAUGGGAUUCUUCAGAGGUCUAC
CACUGGUGCAUACUCUAAUCACAGUGUCGAAGAUCCUGCAUCACAAGAU
GUUACAUUCUGUUCUUCAAGCACCUAUGUCAACCCUCAACACGUUGAAG
GCAGGUGGGAUUCUGAACAGGUUCUCCAAGGAUAUAGCCAUCCUGGAUG
ACCUUCUGCCUCUUACCAUCUUUGACUUCAUCCAGUUGUUACUGAUCGU
GAUUGGAGCUAUAGCAGUUGUCGCAGUGUUACAACCCUACAUCUUCGUU
GCAACAGUGCCAGUGAUAGUGGCUUUCAUUAUGUUGAGAGCAUAUUUCC
UCCAAACCUCACAGCAACUCAAGCAGCUGGAAUCUGAAGGCAGGAGUCC
AAUUUUCACUCAUCUUGUUACAAGCCUGAAGGGACUCUGGACAUUGCGU
GCCUUCGGACGGCAGCCUUACUUUGAAACUCUGUUCCACAAAGCUCUGA
AUUUACAUACUGCCAACUGGUUCUUGUACCUGUCAACACUGCGCUGGUU
CCAAAUGAGAAUAGAAAUGAUUUUUGUCAUCUUCUUCAUUGCUGUUACC
UUCAUUUCCAUUUUAACAACAGGAGAAGGAGAAGGAAGAGUUGGUAUUA
UCCUGACUUUAGCCAUGAACAUCAUGAGUACAUUGCAGUGGGCUGUGAA
CUCCAGCAUAGAUGUGGAUAGCUUGAUGCGAUCUGUGAGCCGAGUCUUC
AAGUUCAUUGACAUGCCCACCGAGGGUAAGCCUACCAAGUCCACCAAGC
CCUACAAGAAUGGCCAACUCUCGAAGGUUAUGAUCAUUGAGAAUUCACA
CGUGAAGAAAGAUGACAUCUGGCCCUCAGGGGGCCAAAUGACUGUCAAA
GAUCUCACAGCCAAGUACACAGAAGGUGGAAAUGCCAUCCUGGAGAACA
UUUCCUUCAGCAUCAGUCCUGGCCAGAGGGUGGGCCUCUUGGGAAGAAC
UGGAUCAGGGAAGAGUACUUUGUUAUCAGCCUUCUUGAGACUACUGAAC
ACUGAAGGCGAGAUCCAGAUCGAUGGUGUGUCUUGGGACAGCAUCACUU
UGCAACAGUGGAGGAAGGCCUUCGGCGUGAUACCACAGAAGGUGUUCAU
CUUCUCCGGAACCUUCAGGAAGAACUUGGAUCCCUAUGAACAGUGGAGU
GAUCAGGAGAUCUGGAAGGUUGCAGAUGAGGUUGGGCUCAGAUCUGUGA
UAGAACAGUUUCCUGGGAAGCUUGACUUUGUCCUUGUGGAUGGGGGCUG
UGUCCUAAGCCACGGCCACAAGCAGUUGAUGUGCUUGGCUAGAUCUGUU
CUCAGUAAGGCGAAGAUCUUGCUGCUUGAUGAACCCAGUGCUCAUUUGG
AUCCAGUAACAUACCAGAUCAUUCGGAGAACUCUGAAGCAGGCAUUUGC
UGAUUGCACAGUAAUUCUCUGUGAACACAGGAUAGAAGCAAUGCUGGAA
UGCCAACAGUUCUUGGUCAUCGAAGAGAACAAGGUGCGGCAGUACGAUU
CCAUCCAGAAGCUGCUGAACGAGAGGAGCCUCUUCCGGCAAGCCAUCAG
CCCCUCCGACAGGGUGAAGCUCUUUCCCCACCGGAACUCAAGCAAGUGC
AAGUCUAAGCCCCAGAUCGCCGCUCUGAAGGAAGAGACUGAGGAAGAGG
UGCAGGAUACCAGGCUGUGAUAAUAGCUCGAGCUAGUGACUGACUAGGA
UCUGGUUACCACUAAACCAGCCUCAAGAACACCCGAAUGGAGUCUCUAA
GCUACAUAAUACCAACUUACACUUACAAAAUGUUGUCCCCCAAAAUGUA
GCCAUUCGUAUCUGCUCCUAAUAAAAAGAAAGUUUCUUCACAUUCUAG
(mARM1831)
SEQ ID NO: 49
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGCGCAGCC
CCCUCGAGAAGGCCAGCGUGGUGAGCAAGCUGUUCUUCAGCUGGACCCG
CCCCAUCCUGCGCAAGGGCUACCGCCAGCGCCUGGAGCUGAGCGACAUC
UACCAGAUCCCCAGCGUGGACAGCGCCGACAACCUGAGCGAGAAGCUGG
AGCGCGAGUGGGACCGCGAGCUGGCCAGCAAGAAGAACCCCAAGCUGAU
CAACGCCCUGCGCCGCUGCUUCUUCUGGCGCUUCAUGUUCUACGGCAUC
UUCCUGUACCUGGGCGAGGUGACCAAGGCCGUGCAGCCCCUGCUGCUGG
GCCGCAUCAUCGCCAGCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCCGCCAUCUUCGGCCUGCACCACAUCGGCAUGCAGA
UGCGCAUCGCCAUGUUCAGCCUGAUCUACAAGAAGACCCUGAAGCUGAG
CAGCCGCGUGCUGGACAAGAUCAGCAUCGGCCAGCUGGUGAGCCUGCUG
AGCAACAACCUGAACAAGUUCGACGAGGGCCUGGCCCUGGCCCACUUCG
UGUGGAUCGCCCCCCUGCAGGUGGCCCUGCUGAUGGGCCUGAUCUGGGA
GCUGCUGCAGGCCAGCGCCUUCUGCGGCCUGGGCUUCCUGAUCGUGCUG
GCCCUGUUCCAGGCCGGCCUGGGCCGCAUGAUGAUGAAGUACCGCGACC
AGCGCGCCGGCAAGAUCAGCGAGCGCCUGGUGAUCACCAGCGAGAUGAU
CGAGAACAUCCAGAGCGUGAAGGCCUACUGCUGGGAGGAGGCCAUGGAG
AAGAUGAUCGAGAACCUGCGCCAGACCGAGCUGAAGCUGACCCGCAAGG
CCGCCUACGUGCGCUACUUCAACAGCAGCGCCUUCUUCUUCAGCGGCUU
CUUCGUGGUGUUCCUGAGCGUGCUGCCCUACGCCCUGAUCAAGGGCAUC
AUCCUGCGCAAGAUCUUCACCACCAUCAGCUUCUGCAUCGUGCUGCGCA
UGGCCGUGACCCGCCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGCUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGCAGCACCGGCGC
CGGCAAGACCAGCCUGCUGAUGGUGAUCAUGGGCGAGCUGGAGCCCAGC
GAGGGCAAGAUCAAGCACAGCGGCCGCAUCAGCUUCUGCAGCCAGUUCA
GCUGGAUCAUGCCCGGCACCAUCAAGGAGAACAUCAUCUUCGGCGUGAG
CUACGACGAGUACCGCUACCGCAGCGUGAUCAAGGCCUGCCAGCUGGAG
GAGGACAUCAGCAAGUUCGCCGAGAAGGACAACAUCGUGCUGGGCGAGG
GCGGCAUCACCCUGAGCGGCGGCCAGCGCGCCCGCAUCAGCCUGGCCCG
CGCCGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGCGCCG
CAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GCGAGUUCGGCGAGAAGCGCAAGAACAGCAUCCUGAACCCCAUCAACAG
CAUCCGCAAGUUCAGCAUCGUGCAGAAGACCCCCCUGCAGAUGAACGGC
AUCGAGGAGGACAGCGACGAGCCCCUGGAGCGCCGCCUGAGCCUGGUGC
CCGACAGCGAGCAGGGCGAGGCCAUCCUGCCCCGCAUCAGCGUGAUCAG
CACCGGCCCCACCCUGCAGGCCCGCCGCCGCCAGAGCGUGCUGAACCUG
AUGACCCACAGCGUGAACCAGGGCCAGAACAUCCACCGCAAGACCACCG
CCAGCACCCGCAAAGUGAGCCUGGCCCCCCAGGCCAACCUGACCGAGCU
GGACAUCUACAGCCGCCGCCUGAGCCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCCCUGCAGGACAAGGGCAACAGCACCCACAGCCGCAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCCGCGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGCAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCCGUGAUCGUGGCCUUCAUCAUGCUGCGCGCCUACUUCC
UGCAGACCAGCCAGCAGCUGAAGCAGCUGGAGAGCGAGGGCCGCAGCCC
CAUCUUCACCCACCUGGUGACCAGCCUGAAGGGCCUGUGGACCCUGCGC
GCCUUCGGCCGCCAGCCCUACUUCGAGACCCUGUUCCACAAGGCCCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGCGAGGGCCGCGUGGGCAUCA
UCCUGACCCUGGCCAUGAACAUCAUGAGCACCCUGCAGUGGGCCGUGAA
CAGCAGCAUCGACGUGGACAGCCUGAUGCGCAGCGUGAGCCGCGUGUUC
AAGUUCAUCGACAUGCCCACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGCGCGUGGGCCUGCUGGGCCGCAC
CGGCAGCGGCAAGAGCACCCUGCUGAGCGCCUUCCUGCGCCUGCUGAAC
ACCGAGGGCGAGAUCCAGAUCGACGGCGUGAGCUGGGACAGCAUCACCC
UGCAGCAGUGGCGCAAGGCCUUCGGCGUGAUCCCCCAGAAGGUGUUCAU
CUUCAGCGGCACCUUCCGCAAGAACCUGGACCCCUACGAGCAGUGGAGC
GACCAGGAGAUCUGGAAGGUGGCCGACGAGGUGGGCCUGCGCAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCCGCAGCGUG
CUGAGCAAGGCCAAGAUCCUGCUGCUGGACGAGCCCAGCGCCCACCUGG
ACCCCGUGACCUACCAGAUCAUCCGCCGCACCCUGAAGCAGGCCUUCGC
CGACUGCACCGUGAUCCUGUGCGAGCACCGCAUCGAGGCCAUGCUGGAG
UGCCAGCAGUUCCUGGUGAUCGAGGAGAACAAGGUGCGCCAGUACGACA
GCAUCCAGAAGCUGCUGAACGAGCGCAGCCUGUUCCGCCAGGCCAUCAG
CCCCAGCGACCGCGUGAAGCUUUUCCCCCACCGCAACAGCAGCAAGUGC
AAGAGCAAGCCCCAGAUCGCCGCCCUGAAGGAGGAGACCGAGGAGGAGG
UGCAGGACACCCGCCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM1832)
SEQ ID NO: 50
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUUGUCUCCAAGCUGUUCUUCAGCUGGACCAG
ACCAAUUUUGAGGAAAGGAUACAGACAGCGCCUGGAAUUGUCAGACAUA
UACCAAAUCCCUUCUGUUGAUUCUGCUGACAAUCUAUCUGAGAAGUUGG
AAAGAGAAUGGGAUAGAGAGCUGGCUUCCAAGAAGAACCCUAAGCUCAU
UAAUGCCCUUCGGCGAUGCUUUUUCUGGAGGUUCAUGUUCUAUGGAAUC
UUCCUGUACUUAGGGGAGGUCACCAAGGCAGUACAGCCUCUCUUGCUGG
GCAGAAUCAUAGCUUCCUAUGACCCGGAUAACAAGGAGGAACGCUCUAU
CGCGAUUUAUCUAGGCAUAGGCUUAUGCCUUCUCUUUAUUGUGAGGACA
CUGCUCCUACACCCAGCCAUUUUUGGCCUUCAUCACAUUGGAAUGCAGA
UGAGAAUAGCUAUGUUUAGUUUGAUUUAUAAGAAGACUUUAAAGCUGUC
AAGCCGUGUUCUAGAUAAAAUAAGUAUUGGACAACUUGUUAGUCUCCUU
UCCAACAACCUGAACAAAUUUGAUGAAGGACUUGCAUUGGCACAUUUCG
UGUGGAUCGCUCCUCUGCAAGUGGCACUCCUGAUGGGGUUGAUCUGGGA
GUUGCUGCAGGCGAGCGCCUUCUGUGGACUUGGCUUCCUGAUAGUCCUU
GCCCUGUUCCAGGCUGGGCUAGGGAGAAUGAUGAUGAAGUACAGAGAUC
AGAGGGCUGGGAAGAUCAGCGAGAGACUCGUGAUCACCUCUGAGAUGAU
CGAGAACAUCCAGUCUGUGAAGGCAUACUGCUGGGAAGAGGCAAUGGAG
AAGAUGAUUGAGAACUUAAGACAGACAGAGCUGAAGCUGACUCGGAAGG
CAGCCUAUGUGAGAUACUUCAACAGCUCAGCCUUCUUCUUCAGCGGGUU
CUUUGUGGUCUUCCUGUCUGUGCUUCCCUAUGCACUAAUCAAGGGAAUC
AUUCUGCGGAAGAUCUUCACAACCAUCUCCUUCUGCAUUGUGCUGCGCA
UGGCGGUCACUCGGCAGUUUCCCUGGGCUGUACAGACAUGGUAUGACUC
UCUGGGAGCCAUCAACAAGAUACAGGAUUUCCUGCAGAAGCAAGAGUAU
AAGACAUUGGAGUACAACUUAACGACUACAGAAGUAGUGAUGGAGAACG
UAACCGCCUUCUGGGAGGAGGGAUUUGGGGAGUUGUUCGAGAAAGCAAA
GCAGAACAACAAUAAUCGGAAGACCUCCAAUGGUGAUGACAGCCUCUUC
UUCAGUAACUUCAGCCUUCUUGGUACUCCUGUCCUGAAGGACAUCAACU
UCAAGAUAGAGAGGGGACAGUUGUUGGCGGUUGCUGGAUCCACUGGAGC
AGGCAAGACUUCACUUCUAAUGGUGAUCAUGGGAGAACUGGAGCCUAGC
GAGGGCAAGAUCAAGCACAGUGGAAGGAUCUCAUUCUGUUCUCAGUUUU
CCUGGAUUAUGCCUGGCACCAUUAAGGAGAACAUCAUCUUUGGUGUUUC
CUAUGAUGAGUACCGCUACAGAAGCGUCAUCAAGGCAUGCCAACUAGAA
GAGGACAUCUCCAAGUUUGCAGAGAAGGACAAUAUAGUUCUUGGAGAAG
GUGGAAUCACACUGAGUGGAGGUCAACGAGCAAGAAUCUCUUUAGCAAG
AGCAGUAUACAAGGACGCUGAUUUGUACUUGUUAGACUCUCCCUUUGGA
UACCUAGAUGUGCUGACCGAGAAGGAGAUAUUCGAAAGCUGUGUCUGUA
AGCUGAUGGCUAACAAGACUAGGAUCUUGGUCACUUCUAAGAUGGAACA
CCUGAAGAAAGCUGACAAGAUCUUGAUCCUGCAUGAAGGUUCUAGCUAC
UUCUACGGGACAUUUUCAGAACUCCAGAAUCUACAGCCAGACUUUAGCU
CAAAGCUCAUGGGAUGUGAUUCUUUCGACCAGUUUAGUGCAGAGAGACG
GAACUCAAUCCUAACUGAGACAUUACACCGUUUCUCAUUAGAAGGAGAU
GCUCCUGUCUCCUGGACAGAGACGAAGAAACAGUCUUUUAAACAGACUG
GAGAGUUUGGGGAGAAACGCAAGAACAGCAUUCUCAAUCCAAUCAACUC
UAUACGAAAGUUCUCCAUUGUGCAGAAGACUCCCUUACAGAUGAAUGGC
AUCGAAGAGGAUUCUGAUGAGCCUUUAGAGAGAAGGCUGUCCUUAGUAC
CAGAUUCUGAGCAGGGAGAGGCGAUACUGCCUCGCAUCAGCGUGAUCAG
CACUGGCCCCACGCUUCAGGCACGAAGGCGCCAGUCUGUCCUGAACCUG
AUGACACACUCAGUUAACCAAGGUCAGAACAUUCACCGAAAGACAACCG
CAUCCACAAGGAAGGUGUCACUGGCCCCUCAGGCAAACUUGACUGAACU
GGACAUCUACUCCAGAAGGUUAUCUCAGGAGACUGGCUUGGAGAUCAGU
GAAGAGAUUAACGAAGAGGACUUAAAGGAGUGCUUCUUUGAUGAUAUGG
AGAGCAUACCAGCAGUGACUACAUGGAACACAUACCUUAGGUACAUCAC
UGUCCACAAGAGCCUGAUCUUCGUGCUAAUUUGGUGCUUGGUGAUCUUC
CUGGCAGAGGUGGCUGCUUCUUUGGUUGUGCUGUGGCUCCUUGGAAACA
CUCCUCUUCAAGACAAAGGGAAUAGUACUCAUUCCAGCAACAAUUCCUA
UGCAGUGAUUAUCACCAGCACCAGUUCGUAUUAUGUGUUCUACAUUUAC
GUGGGAGUAGCCGACACUUUGCUUGCUAUGGGAUUCUUCAGAGGUCUAC
CACUGGUGCAUACUCUAAUCACAGUGUCGAAGAUCCUGCAUCACAAGAU
GUUACAUUCUGUUCUUCAAGCACCUAUGUCAACCCUCAACACGUUGAAG
GCAGGUGGGAUUCUGAACAGGUUCUCCAAGGAUAUAGCCAUCCUGGAUG
ACCUUCUGCCUCUUACCAUCUUUGACUUCAUCCAGUUGUUACUGAUCGU
GAUUGGAGCUAUAGCAGUUGUCGCAGUGUUACAACCCUACAUCUUCGUU
GCAACAGUGCCAGUGAUAGUGGCUUUCAUUAUGUUGAGAGCAUAUUUCC
UCCAAACCUCACAGCAACUCAAGCAGCUGGAAUCUGAAGGCAGGAGUCC
AAUUUUCACUCAUCUUGUUACAAGCCUGAAGGGACUCUGGACAUUGCGU
GCCUUCGGACGGCAGCCUUACUUUGAAACUCUGUUCCACAAAGCUCUGA
AUUUACAUACUGCCAACUGGUUCUUGUACCUGUCAACACUGCGCUGGUU
CCAAAUGAGAAUAGAAAUGAUUUUUGUCAUCUUCUUCAUUGCUGUUACC
UUCAUUUCCAUUUUAACAACAGGAGAAGGAGAAGGAAGAGUUGGUAUUA
UCCUGACUUUAGCCAUGAACAUCAUGAGUACAUUGCAGUGGGCUGUGAA
CUCCAGCAUAGAUGUGGAUAGCUUGAUGCGAUCUGUGAGCCGAGUCUUC
AAGUUCAUUGACAUGCCCACCGAGGGUAAGCCUACCAAGUCCACCAAGC
CCUACAAGAAUGGCCAACUCUCGAAGGUUAUGAUCAUUGAGAAUUCACA
CGUGAAGAAAGAUGACAUCUGGCCCUCAGGGGGCCAAAUGACUGUCAAA
GAUCUCACAGCCAAGUACACAGAAGGUGGAAAUGCCAUCCUGGAGAACA
UUUCCUUCAGCAUCAGUCCUGGCCAGAGGGUGGGCCUCUUGGGAAGAAC
UGGAUCAGGGAAGAGUACUUUGUUAUCAGCCUUCUUGAGACUACUGAAC
ACUGAAGGCGAGAUCCAGAUCGAUGGUGUGUCUUGGGACAGCAUCACUU
UGCAACAGUGGAGGAAGGCCUUCGGCGUGAUACCACAGAAGGUGUUCAU
CUUCUCCGGAACCUUCAGGAAGAACUUGGAUCCCUAUGAACAGUGGAGU
GAUCAGGAGAUCUGGAAGGUUGCAGAUGAGGUUGGGCUCAGAUCUGUGA
UAGAACAGUUUCCUGGGAAGCUUGACUUUGUCCUUGUGGAUGGGGGCUG
UGUCCUAAGCCACGGCCACAAGCAGUUGAUGUGCUUGGCUAGAUCUGUU
CUCAGUAAGGCGAAGAUCUUGCUGCUUGAUGAACCCAGUGCUCAUUUGG
AUCCAGUAACAUACCAGAUCAUUCGGAGAACUCUGAAGCAGGCAUUUGC
UGAUUGCACAGUAAUUCUCUGUGAACACAGGAUAGAAGCAAUGCUGGAA
UGCCAACAGUUCUUGGUCAUCGAAGAGAACAAGGUGCGGCAGUACGAUU
CCAUCCAGAAGCUGCUGAACGAGAGGAGCCUCUUCCGGCAAGCCAUCAG
CCCCUCCGACAGGGUGAAGCUCUUUCCCCACCGGAACUCAAGCAAGUGC
AAGUCUAAGCCCCAGAUCGCCGCUCUGAAGGAAGAGACUGAGGAAGAGG
UGCAGGAUACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM1833)
SEQ ID NO: 51
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCCAGCGUGGUGAGCAAGCUGUUCUUCAGCUGGACCAG
GCCCAUCCUGAGGAAGGGCUACAGGCAGAGGCUGGAGCUGAGCGACAUC
UACCAGAUCCCCAGCGUGGACAGCGCCGACAACCUGAGCGAGAAGCUGG
AGAGGGAGUGGGACAGGGAGCUGGCCAGCAAGAAGAACCCCAAGCUGAU
CAACGCCCUGCGGAGGUGCUUCUUCUGGAGGUUCAUGUUCUACGGCAUC
UUCCUGUACCUGGGCGAGGUGACCAAGGCCGUGCAGCCCCUGCUGCUGG
GCAGGAUCAUCGCCAGCUACGACCCCGACAACAAGGAGGAGAGGAGCAU
CGCCAUCUACCUGGGCAUCGGCCUGUGCCUGCUGUUCAUCGUGAGGACC
CUGCUGCUGCACCCCGCCAUCUUCGGCCUGCACCACAUCGGCAUGCAGA
UGAGGAUCGCCAUGUUCAGCCUGAUCUACAAGAAGACCCUGAAGCUGAG
CAGCAGGGUGCUGGACAAGAUCAGCAUCGGCCAGCUGGUGAGCCUGCUG
AGCAACAACCUGAACAAGUUCGACGAGGGCCUGGCCCUGGCCCACUUCG
UGUGGAUCGCCCCCCUGCAGGUGGCCCUGCUGAUGGGCCUGAUCUGGGA
GCUGCUGCAGGCCAGCGCCUUCUGCGGCCUGGGCUUCCUGAUCGUGCUG
GCCCUGUUCCAGGCCGGCCUGGGCAGGAUGAUGAUGAAGUACAGGGACC
AGAGGGCCGGCAAGAUCAGCGAGAGGCUGGUGAUCACCAGCGAGAUGAU
CGAGAACAUCCAGAGCGUGAAGGCCUACUGCUGGGAGGAGGCCAUGGAG
AAGAUGAUCGAGAACCUGAGGCAGACCGAGCUGAAGCUGACCCGGAAGG
CCGCCUACGUGAGGUACUUCAACAGCAGCGCCUUCUUCUUCAGCGGCUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGAGGA
UGGCCGUGACCCGGCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGCUUCGGCGAGCUGUUCGAGAAGGCCAA
GCAGAACAACAACAACAGGAAGACCAGCAACGGCGACGACAGCCUGUUC
UUCAGCAACUUCAGCCUGCUGGGCACCCCCGUGCUGAAGGACAUCAACU
UCAAGAUCGAGAGGGGCCAGCUGCUGGCCGUGGCCGGCAGCACCGGCGC
CGGCAAGACCAGCCUGCUGAUGGUGAUCAUGGGCGAGCUGGAGCCCAGC
GAGGGCAAGAUCAAGCACAGCGGCAGGAUCAGCUUCUGCAGCCAGUUCA
GCUGGAUCAUGCCCGGCACCAUCAAGGAGAACAUCAUCUUCGGCGUGAG
CUACGACGAGUACAGGUACAGGAGCGUGAUCAAGGCCUGCCAGCUGGAG
GAGGACAUCAGCAAGUUCGCCGAGAAGGACAACAUCGUGCUGGGCGAGG
GCGGCAUCACCCUGAGCGGCGGCCAGAGGGCCAGGAUCAGCCUGGCCAG
GGCCGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGAGGAG
GAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GCGAGUUCGGCGAGAAGAGGAAGAACAGCAUCCUGAACCCCAUCAACAG
CAUCAGGAAGUUCAGCAUCGUGCAGAAGACCCCCCUGCAGAUGAACGGC
AUCGAGGAGGACAGCGACGAGCCCCUGGAGAGGAGGCUGAGCCUGGUGC
CCGACAGCGAGCAGGGCGAGGCCAUCCUGCCCAGGAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCCCAGGCCAACCUGACCGAGCU
GGACAUCUACAGCAGGAGGCUGAGCCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCCCUGCAGGACAAGGGCAACAGCACCCACAGCAGGAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCAGGGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGCAUCCUGAACAGGUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCCGUGAUCGUGGCCUUCAUCAUGCUGAGGGCCUACUUCC
UGCAGACCAGCCAGCAGCUGAAGCAGCUGGAGAGCGAGGGCAGGAGCCC
CAUCUUCACCCACCUGGUGACCAGCCUGAAGGGCCUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGAGGUGGUU
CCAGAUGAGGAUCGAGAUGAUCUUCGUGAUCUUCUUCAUCGCCGUGACC
UUCAUCAGCAUCCUGACCACCGGCGAGGGCGAGGGCAGGGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCCACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGCAGGAC
CGGCAGCGGCAAGAGCACCCUGCUGAGCGCCUUCCUGAGGCUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCCCAGAAGGUGUUCAU
CUUCAGCGGCACCUUCAGGAAGAACCUGGACCCCUACGAGCAGUGGAGC
GACCAGGAGAUCUGGAAGGUGGCCGACGAGGUGGGCCUGAGGAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCAGGAGCGUG
CUGAGCAAGGCCAAGAUCCUGCUGCUGGACGAGCCCAGCGCCCACCUGG
ACCCCGUGACCUACCAGAUCAUCAGGAGGACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM1834)
SEQ ID NO: 52
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CCCUGGAGAAGGCCAGCGUGGUGUCCAAGCUGUUCUUCAGCUGGACCAG
GCCCAUCCUGAGGAAGGGCUACAGGCAGAGGCUGGAGCUGUCAGACAUC
UACCAGAUCCCCUCUGUGGACAGCGCUGACAACCUGUCUGAGAAGCUGG
AGAGGGAGUGGGACAGGGAGCUGGCCAGCAAGAAGAACCCCAAGCUGAU
CAACGCCCUGCGGAGGUGCUUCUUCUGGAGGUUCAUGUUCUACGGAAUC
UUCCUGUACCUGGGGGAGGUGACCAAGGCCGUGCAGCCCCUGCUGCUGG
GAAGGAUCAUCGCCUCCUACGACCCCGACAACAAGGAGGAGAGGAGCAU
CGCCAUCUACCUGGGCAUCGGCCUGUGCCUGCUGUUCAUCGUGAGGACC
CUGCUGCUGCACCCCGCCAUCUUCGGCCUGCACCACAUCGGAAUGCAGA
UGAGGAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCCCUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGGAUGAUGAUGAAGUACAGGGACC
AGAGGGCUGGCAAGAUCAGCGAGAGGCUGGUGAUCACCUCAGAGAUGAU
CGAGAACAUCCAGAGCGUGAAGGCAUACUGCUGGGAGGAGGCCAUGGAG
AAGAUGAUCGAGAACCUGAGGCAGACCGAGCUGAAGCUGACCCGGAAGG
CCGCCUACGUGAGGUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGAGGA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGGAAGACCAGCAACGGCGACGACAGCCUGUUC
UUCAGCAACUUCAGCCUGCUGGGCACCCCCGUGCUGAAGGACAUCAACU
UCAAGAUCGAGAGGGGACAGCUGCUGGCCGUGGCCGGAAGCACCGGAGC
CGGCAAGACCUCACUGCUGAUGGUGAUCAUGGGAGAGCUGGAGCCCUCA
GAGGGCAAGAUCAAGCACAGUGGAAGGAUCUCAUUCUGCUCUCAGUUCA
GCUGGAUCAUGCCCGGCACCAUCAAGGAGAACAUCAUCUUCGGUGUGUC
CUACGACGAGUACAGGUACAGGAGCGUGAUCAAGGCCUGCCAGCUGGAG
GAGGACAUCAGCAAGUUCGCAGAGAAGGACAACAUCGUGCUGGGAGAGG
GUGGCAUCACCCUGAGCGGAGGCCAGAGGGCCAGGAUCAGCCUGGCAAG
GGCAGUGUACAAGGACGCUGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCCGACUUCAGCA
GCAAGCUGAUGGGCUGCGACAGCUUCGACCAGUUCAGCGCCGAGAGGAG
GAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCCAUCAACAG
CAUCAGGAAGUUCAGCAUCGUGCAGAAGACCCCCCUGCAGAUGAACGGC
AUCGAGGAGGACAGCGACGAGCCCCUGGAGAGGAGGCUGUCCCUGGUGC
CCGACAGCGAGCAGGGCGAGGCCAUCCUGCCCAGGAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCCCAGGCCAACCUGACCGAGCU
GGACAUCUACAGCAGGAGGCUGAGCCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCCCUGCAGGACAAGGGCAACAGCACCCACAGCAGGAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGGGGUCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGUGGGAUCCUGAACAGGUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCCGUGAUCGUGGCCUUCAUCAUGCUGAGGGCCUACUUCC
UGCAGACCAGCCAGCAGCUGAAGCAGCUGGAGUCUGAGGGCAGGAGUCC
CAUCUUCACCCACCUGGUGACCAGCCUGAAGGGACUGUGGACCCUGAGG
GCCUUCGGCCGGCAGCCCUACUUCGAGACCCUGUUCCACAAGGCUCUGA
ACCUGCACACCGCCAACUGGUUCCUGUACCUGAGCACCCUGAGGUGGUU
CCAGAUGAGGAUCGAGAUGAUCUUCGUGAUCUUCUUCAUCGCCGUGACC
UUCAUCUCCAUCCUGACCACCGGCGAGGGAGAGGGAAGGGUGGGCAUCA
UCCUGACCCUGGCCAUGAACAUCAUGAGCACCCUGCAGUGGGCUGUGAA
CAGCAGCAUCGACGUGGACAGCCUGAUGAGGAGCGUGAGCAGGGUGUUC
AAGUUCAUCGACAUGCCCACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCUCAAUCAGCCCCGGCCAGAGGGUGGGCCUGCUGGGAAGGAC
CGGCUCAGGCAAGAGCACCCUGCUGAGCGCCUUCCUGAGGCUGCUGAAC
ACCGAGGGCGAGAUCCAGAUCGACGGCGUGAGCUGGGACUCAAUCACCC
UGCAGCAGUGGAGGAAGGCCUUCGGCGUGAUCCCCCAGAAGGUGUUCAU
CUUCUCUGGAACCUUCAGGAAGAACCUGGACCCCUACGAGCAGUGGAGC
GACCAGGAGAUCUGGAAGGUGGCCGACGAGGUGGGCCUGAGGAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGGAGCGUG
CUGAGCAAGGCCAAGAUCCUGCUGCUGGACGAGCCCAGUGCCCACCUGG
ACCCCGUGACCUACCAGAUCAUCAGGAGGACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM1835)
SEQ ID NO: 53
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM1836)
SEQ ID NO: 54
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUUUUCAGCUGGACCAG
ACCAAUCCUGAGGAAGGGCUACAGACAGCGCCUGGAGCUGUCAGACAUC
UACCAGAUCCCUUCUGUGGAUUCUGCUGACAAUCUGUCUGAGAAGCUGG
AGAGAGAGUGGGAUAGAGAGCUGGCCAGCAAGAAGAAUCCUAAGCUGAU
CAAUGCCCUGCGGAGGUGCUUUUUCUGGAGAUUUAUGUUCUACGGAAUC
UUUCUGUACCUGGGGGAGGUGACCAAGGCCGUGCAGCCUCUGCUGCUGG
GAAGAAUCAUCGCCUCCUACGACCCCGAUAACAAGGAGGAGCGCUCUAU
CGCCAUCUACCUGGGCAUCGGCCUGUGCCUGCUGUUUAUCGUGAGGACA
CUGCUGCUGCACCCAGCCAUCUUUGGCCUGCACCACAUCGGAAUGCAGA
UGAGAAUCGCCAUGUUUAGCCUGAUCUACAAGAAGACCCUGAAGCUGUC
AAGCAGGGUGCUGGAUAAGAUCAGUAUCGGACAGCUGGUGAGUCUGCUG
UCCAACAACCUGAACAAGUUUGAUGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUUCAGGCCGGGCUGGGGAGAAUGAUGAUGAAGUACAGAGAUC
AGAGAGCUGGGAAGAUCAGCGAGAGACUGGUGAUCACCUCAGAGAUGAU
CGAGAAUAUCCAGUCUGUGAAGGCAUACUGCUGGGAGGAGGCCAUGGAG
AAGAUGAUCGAGAACCUGAGACAGACAGAGCUGAAGCUGACCCGGAAGG
CCGCCUACGUGAGAUACUUCAAUAGCAGCGCCUUCUUCUUCUCAGGGUU
CUUUGUGGUGUUUCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUUCCCUGGGCCGUGCAGACAUGGUACGACUC
UCUGGGAGCCAUCAACAAGAUCCAGGAUUUCCUGCAGAAGCAGGAGUAC
AAGACACUGGAGUACAACCUGACCACCACAGAGGUGGUGAUGGAGAAUG
UGACAGCCUUCUGGGAGGAGGGAUUUGGGGAGCUGUUUGAGAAGGCCAA
GCAGAACAAUAACAAUAGAAAGACCUCUAAUGGCGAUGACAGCCUGUUC
UUCAGUAAUUUCAGCCUGCUGGGCACCCCUGUGCUGAAGGAUAUCAAUU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUUU
CCUGGAUCAUGCCUGGCACCAUCAAGGAGAAUAUCAUCUUUGGUGUGUC
CUACGAUGAGUACAGAUACAGAAGCGUGAUCAAGGCCUGCCAGCUGGAG
GAGGACAUCUCCAAGUUUGCAGAGAAGGACAAUAUCGUGCUGGGAGAGG
GUGGCAUCACACUGAGCGGAGGCCAGAGGGCCAGAAUCUCUCUGGCAAG
AGCAGUGUACAAGGAUGCUGAUCUGUACCUGCUGGACUCUCCUUUUGGA
UACCUGGAUGUGCUGACAGAGAAGGAGAUCUUUGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUUUACGGGACAUUUAGCGAGCUGCAGAAUCUGCAGCCAGACUUUAGCA
GCAAGCUGAUGGGCUGCGAUUCUUUCGACCAGUUUAGCGCCGAGAGAAG
AAAUAGCAUCCUGACCGAGACCCUGCACAGGUUCAGCCUGGAGGGCGAU
GCCCCUGUGUCCUGGACAGAGACAAAGAAGCAGUCUUUUAAGCAGACCG
GAGAGUUUGGGGAGAAGAGGAAGAAUUCUAUCCUGAAUCCAAUCAACUC
UAUCAGGAAGUUUUCCAUCGUGCAGAAGACCCCCCUGCAGAUGAAUGGC
AUCGAGGAGGAUUCUGAUGAGCCUCUGGAGAGAAGGCUGUCCCUGGUGC
CAGAUUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACACACAGCGUGAACCAGGGCCAGAACAUCCACAGGAAGACAACAG
CCUCCACAAGGAAGGUGAGCCUGGCCCCUCAGGCCAACCUGACCGAGCU
GGAUAUCUACAGCAGAAGGCUGUCUCAGGAGACCGGCCUGGAGAUCAGU
GAGGAGAUCAACGAGGAGGACCUGAAGGAGUGCUUUUUUGAUGAUAUGG
AGAGCAUCCCAGCCGUGACCACAUGGAACACAUACCUGAGGUACAUCAC
CGUGCACAAGAGCCUGAUCUUUGUGCUGAUCUGGUGCCUGGUGAUCUUU
CUGGCCGAGGUGGCCGCCUCUCUGGUGGUGCUGUGGCUGCUGGGCAACA
CCCCUCUGCAGGACAAGGGGAAUAGUACCCACAGCAGAAAUAACAGCUA
CGCCGUGAUCAUCACCAGCACCAGUAGCUACUACGUGUUUUACAUCUAC
GUGGGAGUGGCCGACACCCUGCUGGCCAUGGGCUUCUUCAGAGGUCUGC
CACUGGUGCACACCCUGAUCACAGUGAGCAAGAUCCUGCACCACAAGAU
GCUGCACUCUGUGCUGCAGGCCCCUAUGAGCACCCUGAACACCCUGAAG
GCCGGUGGGAUCCUGAAUAGAUUCUCCAAGGAUAUCGCCAUCCUGGAUG
ACCUGCUGCCUCUGACCAUCUUUGACUUCAUCCAGCUGCUGCUGAUCGU
GAUCGGAGCCAUCGCCGUGGUGGCCGUGCUGCAGCCCUACAUCUUUGUG
GCCACAGUGCCAGUGAUCGUGGCCUUUAUCAUGCUGAGAGCCUACUUCC
UGCAGACCAGCCAGCAGCUGAAGCAGCUGGAGUCUGAGGGCAGGAGUCC
AAUCUUCACCCACCUGGUGACAAGCCUGAAGGGACUGUGGACACUGAGG
GCCUUCGGCCGGCAGCCUUACUUUGAGACCCUGUUCCACAAGGCUCUGA
AUCUGCACACCGCCAACUGGUUCCUGUACCUGAGCACACUGCGCUGGUU
CCAGAUGAGAAUCGAGAUGAUCUUUGUGAUCUUCUUCAUCGCCGUGACC
UUCAUCUCCAUCCUGACAACAGGCGAGGGAGAGGGAAGAGUGGGCAUCA
UCCUGACCCUGGCCAUGAAUAUCAUGAGCACACUGCAGUGGGCUGUGAA
CUCCAGCAUCGAUGUGGAUAGCCUGAUGAGGUCUGUGAGCAGGGUGUUU
AAGUUCAUCGACAUGCCAACAGAGGGCAAGCCUACCAAGAGCACCAAGC
CAUACAAGAAUGGCCAGCUGAGCAAGGUGAUGAUCAUCGAGAAUAGCCA
CGUGAAGAAGGAUGACAUCUGGCCCAGCGGGGGCCAGAUGACCGUGAAG
GAUCUGACAGCCAAGUACACAGAGGGCGGCAAUGCCAUCCUGGAGAACA
UCUCCUUCUCAAUCAGCCCUGGCCAGAGGGUGGGCCUGCUGGGAAGAAC
CGGCUCAGGGAAGAGUACCCUGCUGAGCGCCUUUCUGAGACUGCUGAAC
ACCGAGGGCGAGAUCCAGAUCGAUGGCGUGUCUUGGGAUUCAAUCACCC
UGCAGCAGUGGAGGAAGGCCUUUGGCGUGAUCCCACAGAAGGUGUUUAU
CUUUUCUGGAACAUUUAGAAAGAACCUGGAUCCCUACGAGCAGUGGAGC
GAUCAGGAGAUCUGGAAGGUGGCCGAUGAGGUGGGGCUGAGAUCUGUGA
UCGAGCAGUUUCCUGGGAAGCUGGACUUUGUGCUGGUGGAUGGGGGCUG
CGUGCUGAGCCACGGCCACAAGCAGCUGAUGUGCCUGGCCAGAUCUGUG
CUGAGUAAGGCCAAGAUCCUGCUGCUGGAUGAGCCCAGUGCCCACCUGG
AUCCAGUGACAUACCAGAUCAUCAGAAGAACCCUGAAGCAGGCCUUUGC
CGAUUGCACAGUGAUCCUGUGCGAGCACAGGAUCGAGGCCAUGCUGGAG
UGCCAGCAGUUUCUGGUGAUCGAGGAGAACAAGGUGCGGCAGUACGAUU
CCAUCCAGAAGCUGCUGAACGAGAGGAGCCUGUUCCGGCAGGCCAUCAG
CCCCUCCGACAGGGUGAAGCUGUUUCCCCACCGGAACAGCAGCAAGUGC
AAGUCUAAGCCCCAGAUCGCCGCCCUGAAGGAGGAGACAGAGGAGGAGG
UGCAGGAUACAAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM1880)
SEQ ID NO: 55
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCAUGGGCCAGCGCAGCC
CCCUCGAGAAGGCCAGCGUGGUGAGCAAGCUGUUCUUCAGCUGGACCCG
CCCCAUCCUGCGCAAGGGCUACCGCCAGCGCCUGGAGCUGAGCGACAUC
UACCAGAUCCCCAGCGUGGACAGCGCCGACAACCUGAGCGAGAAGCUGG
AGCGCGAGUGGGACCGCGAGCUGGCCAGCAAGAAGAACCCCAAGCUGAU
CAACGCCCUGCGCCGCUGCUUCUUCUGGCGCUUCAUGUUCUACGGCAUC
UUCCUGUACCUGGGCGAGGUGACCAAGGCCGUGCAGCCCCUGCUGCUGG
GCCGCAUCAUCGCCAGCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCCGCCAUCUUCGGCCUGCACCACAUCGGCAUGCAGA
UGCGCAUCGCCAUGUUCAGCCUGAUCUACAAGAAGACCCUGAAGCUGAG
CAGCCGCGUGCUGGACAAGAUCAGCAUCGGCCAGCUGGUGAGCCUGCUG
AGCAACAACCUGAACAAGUUCGACGAGGGCCUGGCCCUGGCCCACUUCG
UGUGGAUCGCCCCCCUGCAGGUGGCCCUGCUGAUGGGCCUGAUCUGGGA
GCUGCUGCAGGCCAGCGCCUUCUGCGGCCUGGGCUUCCUGAUCGUGCUG
GCCCUGUUCCAGGCCGGCCUGGGCCGCAUGAUGAUGAAGUACCGCGACC
AGCGCGCCGGCAAGAUCAGCGAGCGCCUGGUGAUCACCAGCGAGAUGAU
CGAGAACAUCCAGAGCGUGAAGGCCUACUGCUGGGAGGAGGCCAUGGAG
AAGAUGAUCGAGAACCUGCGCCAGACCGAGCUGAAGCUGACCCGCAAGG
CCGCCUACGUGCGCUACUUCAACAGCAGCGCCUUCUUCUUCAGCGGCUU
CUUCGUGGUGUUCCUGAGCGUGCUGCCCUACGCCCUGAUCAAGGGCAUC
AUCCUGCGCAAGAUCUUCACCACCAUCAGCUUCUGCAUCGUGCUGCGCA
UGGCCGUGACCCGCCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGCUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGCAGCACCGGCGC
CGGCAAGACCAGCCUGCUGAUGGUGAUCAUGGGCGAGCUGGAGCCCAGC
GAGGGCAAGAUCAAGCACAGCGGCCGCAUCAGCUUCUGCAGCCAGUUCA
GCUGGAUCAUGCCCGGCACCAUCAAGGAGAACAUCAUCUUCGGCGUGAG
CUACGACGAGUACCGCUACCGCAGCGUGAUCAAGGCCUGCCAGCUGGAG
GAGGACAUCAGCAAGUUCGCCGAGAAGGACAACAUCGUGCUGGGCGAGG
GCGGCAUCACCCUGAGCGGCGGCCAGCGCGCCCGCAUCAGCCUGGCCCG
CGCCGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGCGCCG
CAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GCGAGUUCGGCGAGAAGCGCAAGAACAGCAUCCUGAACCCCAUCAACAG
CAUCCGCAAGUUCAGCAUCGUGCAGAAGACCCCCCUGCAGAUGAACGGC
AUCGAGGAGGACAGCGACGAGCCCCUGGAGCGCCGCCUGAGCCUGGUGC
CCGACAGCGAGCAGGGCGAGGCCAUCCUGCCCCGCAUCAGCGUGAUCAG
CACCGGCCCCACCCUGCAGGCCCGCCGCCGCCAGAGCGUGCUGAACCUG
AUGACCCACAGCGUGAACCAGGGCCAGAACAUCCACCGCAAGACCACCG
CCAGCACCCGCAAAGUGAGCCUGGCCCCCCAGGCCAACCUGACCGAGCU
GGACAUCUACAGCCGCCGCCUGAGCCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCCCUGCAGGACAAGGGCAACAGCACCCACAGCCGCAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCCGCGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGCAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCCGUGAUCGUGGCCUUCAUCAUGCUGCGCGCCUACUUCC
UGCAGACCAGCCAGCAGCUGAAGCAGCUGGAGAGCGAGGGCCGCAGCCC
CAUCUUCACCCACCUGGUGACCAGCCUGAAGGGCCUGUGGACCCUGCGC
GCCUUCGGCCGCCAGCCCUACUUCGAGACCCUGUUCCACAAGGCCCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGCGAGGGCCGCGUGGGCAUCA
UCCUGACCCUGGCCAUGAACAUCAUGAGCACCCUGCAGUGGGCCGUGAA
CAGCAGCAUCGACGUGGACAGCCUGAUGCGCAGCGUGAGCCGCGUGUUC
AAGUUCAUCGACAUGCCCACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGCGCGUGGGCCUGCUGGGCCGCAC
CGGCAGCGGCAAGAGCACCCUGCUGAGCGCCUUCCUGCGCCUGCUGAAC
ACCGAGGGCGAGAUCCAGAUCGACGGCGUGAGCUGGGACAGCAUCACCC
UGCAGCAGUGGCGCAAGGCCUUCGGCGUGAUCCCCCAGAAGGUGUUCAU
CUUCAGCGGCACCUUCCGCAAGAACCUGGACCCCUACGAGCAGUGGAGC
GACCAGGAGAUCUGGAAGGUGGCCGACGAGGUGGGCCUGCGCAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCCGCAGCGUG
CUGAGCAAGGCCAAGAUCCUGCUGCUGGACGAGCCCAGCGCCCACCUGG
ACCCCGUGACCUACCAGAUCAUCCGCCGCACCCUGAAGCAGGCCUUCGC
CGACUGCACCGUGAUCCUGUGCGAGCACCGCAUCGAGGCCAUGCUGGAG
UGCCAGCAGUUCCUGGUGAUCGAGGAGAACAAGGUGCGCCAGUACGACA
GCAUCCAGAAGCUGCUGAACGAGCGCAGCCUGUUCCGCCAGGCCAUCAG
CCCCAGCGACCGCGUGAAGCUUUUCCCCCACCGCAACAGCAGCAAGUGC
AAGAGCAAGCCCCAGAUCGCCGCCCUGAAGGAGGAGACCGAGGAGGAGG
UGCAGGACACCCGCCUGUAGCUCGAGCUAGUGACUGACUAGGAUCUGGU
UACCACUAAACCAGCCUCAAGAACACCCGAAUGGAGUCUCUAAGCUACA
UAAUACCAACUUACACUUACAAAAUGUUGUCCCCCAAAAUGUAGCCAUU
CGUAUCUGCUCCUAAUAAAAAGAAAGUUUCUUCACAUUCUAG
(mARM1947)
SEQ ID NO: 56
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCAUGCAGAGGAGCCCCC
UGGAGAAGGCUAGCGUGGUGAGCAAGCUGUUCUUCAGCUGGACCAGGCC
CAUCCUGAGGAAGGGCUACAGGCAGAGGCUGGAGCUGAGCGACAUCUAC
CAGAUCCCCAGCGUGGACAGCGCCGACAACCUGAGCGAGAAGCUGGAGA
GGGAGUGGGACAGGGAGCUGGCCAGCAAGAAGAACCCCAAGCUGAUCAA
CGCCCUGCGGAGGUGCUUCUUCUGGAGGUUCAUGUUCUACGGCAUCUUC
CUGUACCUGGGCGAGGUGACCAAGGCCGUGCAGCCCCUGCUGCUGGGCA
GGAUCAUCGCCAGCUACGACCCCGACAACAAGGAGGAGAGGAGCAUCGC
CAUCUACCUGGGCAUCGGCCUGUGCCUGCUGUUCAUCGUGAGGACCCUG
CUGCUGCACCCCGCCAUCUUCGGCCUGCACCACAUCGGCAUGCAGAUGA
GGAUCGCCAUGUUCAGCCUGAUCUACAAGAAGACCCUGAAGCUGAGCAG
CAGGGUGCUGGACAAGAUCAGCAUCGGCCAGCUGGUGAGCCUGCUGAGC
AACAACCUGAACAAGUUCGACGAGGGCCUGGCCCUGGCCCACUUCGUGU
GGAUCGCCCCCCUGCAGGUGGCCCUGCUGAUGGGCCUGAUCUGGGAGCU
GCUGCAGGCCAGCGCCUUCUGCGGCCUGGGCUUCCUGAUCGUGCUGGCC
CUGUUCCAGGCCGGCCUGGGCAGGAUGAUGAUGAAGUACAGGGACCAGA
GGGCCGGCAAGAUCAGCGAGAGGCUGGUGAUCACCAGCGAGAUGAUCGA
GAACAUCCAGAGCGUGAAGGCCUACUGCUGGGAGGAGGCCAUGGAGAAG
AUGAUCGAGAACCUGAGGCAGACCGAGCUGAAGCUGACCCGGAAGGCCG
CCUACGUGAGGUACUUCAACAGCAGCGCCUUCUUCUUCAGCGGCUUCUU
CGUGGUGUUCCUGAGCGUGCUGCCCUACGCCCUGAUCAAGGGCAUCAUC
CUGCGGAAGAUCUUCACCACCAUCAGCUUCUGCAUCGUGCUGAGGAUGG
CCGUGACCCGGCAGUUCCCCUGGGCCGUGCAGACCUGGUACGACAGCCU
GGGCGCCAUCAACAAGAUCCAGGACUUCCUGCAGAAGCAGGAGUACAAG
ACCCUGGAGUACAACCUGACCACCACCGAGGUGGUGAUGGAGAACGUGA
CCGCCUUCUGGGAGGAGGGCUUCGGCGAGCUGUUCGAGAAGGCCAAGCA
GAACAACAACAACAGGAAGACCAGCAACGGCGACGACAGCCUGUUCUUC
AGCAACUUCAGCCUGCUGGGCACCCCCGUGCUGAAGGACAUCAACUUCA
AGAUCGAGAGGGGCCAGCUGCUGGCCGUGGCCGGCAGCACCGGCGCCGG
CAAGACCAGCCUGCUGAUGGUGAUCAUGGGCGAGCUGGAGCCCAGCGAG
GGCAAGAUCAAGCACAGCGGCAGGAUCAGCUUCUGCAGCCAGUUCAGCU
GGAUCAUGCCCGGCACCAUCAAGGAGAACAUCAUCUUCGGCGUGAGCUA
CGACGAGUACAGGUACAGGAGCGUGAUCAAGGCCUGCCAGCUGGAGGAG
GACAUCAGCAAGUUCGCCGAGAAGGACAACAUCGUGCUGGGCGAGGGCG
GCAUCACCCUGAGCGGCGGCCAGAGGGCCAGGAUCAGCCUGGCCAGGGC
CGUGUACAAGGACGCCGACCUGUACCUGCUGGACAGCCCCUUCGGCUAC
CUGGACGUGCUGACCGAGAAGGAGAUCUUCGAGAGCUGCGUGUGCAAGC
UGAUGGCCAACAAGACCAGGAUCCUGGUGACCAGCAAGAUGGAGCACCU
GAAGAAGGCCGACAAGAUCCUGAUCCUGCACGAGGGCAGCAGCUACUUC
UACGGCACCUUCAGCGAGCUGCAGAACCUGCAGCCCGACUUCAGCAGCA
AGCUGAUGGGCUGCGACAGCUUCGACCAGUUCAGCGCCGAGAGGAGGAA
CAGCAUCCUGACCGAGACACUGCACAGGUUCAGCCUGGAGGGCGACGCC
CCCGUGAGCUGGACCGAGACAAAGAAGCAGAGCUUCAAGCAGACCGGCG
AGUUCGGCGAGAAGAGGAAGAACAGCAUCCUGAACCCCAUCAACAGCAU
CAGGAAGUUCAGCAUCGUGCAGAAGACCCCCCUGCAGAUGAACGGCAUC
GAGGAGGACAGCGACGAGCCCCUGGAGAGGAGGCUGAGCCUGGUGCCCG
ACAGCGAGCAGGGCGAGGCCAUCCUGCCCAGGAUCAGCGUGAUCAGCAC
CGGCCCCACCCUGCAGGCCAGGAGGAGGCAGAGCGUGCUGAACCUGAUG
ACCCACAGCGUGAACCAGGGCCAGAACAUCCACAGGAAGACCACCGCCA
GCACCAGGAAGGUGAGCCUGGCCCCCCAGGCCAACCUGACCGAGCUGGA
CAUCUACAGCAGGAGGCUGAGCCAGGAGACAGGCCUGGAGAUCAGCGAG
GAGAUCAACGAGGAGGACCUGAAGGAGUGCUUCUUCGACGACAUGGAGA
GCAUCCCCGCCGUGACCACCUGGAACACCUACCUGAGGUACAUCACCGU
GCACAAGAGCCUGAUCUUCGUGCUGAUCUGGUGCCUGGUGAUCUUCCUG
GCCGAGGUGGCCGCCAGCCUGGUGGUGCUGUGGCUGCUGGGCAACACCC
CCCUGCAGGACAAGGGCAACAGCACCCACAGCAGGAACAACAGCUACGC
CGUGAUCAUCACCAGCACCAGCAGCUACUACGUGUUCUACAUCUACGUG
GGCGUGGCCGACACCCUGCUGGCCAUGGGCUUCUUCAGGGGCCUGCCCC
UGGUGCACACCCUGAUCACCGUGAGCAAGAUCCUGCACCACAAGAUGCU
GCACAGCGUGCUGCAGGCCCCCAUGAGCACCCUGAACACCCUGAAGGCC
GGCGGCAUCCUGAACAGGUUCAGCAAGGACAUCGCCAUCCUGGACGACC
UGCUGCCCCUGACCAUCUUCGACUUCAUCCAGCUGCUGCUGAUCGUGAU
CGGCGCCAUCGCCGUGGUGGCCGUGCUGCAGCCCUACAUCUUCGUGGCC
ACCGUGCCCGUGAUCGUGGCCUUCAUCAUGCUGAGGGCCUACUUCCUGC
AGACCAGCCAGCAGCUGAAGCAGCUGGAGAGCGAGGGCAGGAGCCCCAU
CUUCACCCACCUGGUGACCAGCCUGAAGGGCCUGUGGACCCUGAGGGCC
UUCGGCCGGCAGCCCUACUUCGAGACACUGUUCCACAAGGCCCUGAACC
UGCACACCGCCAACUGGUUCCUGUACCUGAGCACCCUGAGGUGGUUCCA
GAUGAGGAUCGAGAUGAUCUUCGUGAUCUUCUUCAUCGCCGUGACCUUC
AUCAGCAUCCUGACCACCGGCGAGGGCGAGGGCAGGGUGGGCAUCAUCC
UGACCCUGGCCAUGAACAUCAUGAGCACCCUGCAGUGGGCCGUGAACAG
CAGCAUCGACGUGGACAGCCUGAUGAGGAGCGUGAGCAGGGUGUUCAAG
UUCAUCGACAUGCCCACCGAGGGCAAGCCCACCAAGAGCACCAAGCCCU
ACAAGAACGGCCAGCUGAGCAAGGUGAUGAUCAUCGAGAACAGCCACGU
GAAGAAGGACGACAUCUGGCCCAGCGGCGGCCAGAUGACCGUGAAGGAC
CUGACCGCCAAGUACACCGAGGGCGGCAACGCCAUCCUGGAGAACAUCA
GCUUCAGCAUCAGCCCCGGCCAGAGGGUGGGCCUGCUGGGCAGGACCGG
CAGCGGCAAGAGCACCCUGCUGAGCGCCUUCCUGAGGCUGCUGAACACC
GAGGGCGAGAUCCAGAUCGACGGCGUGAGCUGGGACAGCAUCACCCUGC
AGCAGUGGAGGAAGGCCUUCGGCGUGAUCCCCCAGAAGGUGUUCAUCUU
CAGCGGCACCUUCAGGAAGAACCUGGACCCCUACGAGCAGUGGAGCGAC
CAGGAGAUCUGGAAGGUGGCCGACGAGGUGGGCCUGAGGAGCGUGAUCG
AGCAGUUCCCCGGCAAGCUGGACUUCGUGCUGGUGGACGGCGGCUGCGU
GCUGAGCCACGGCCACAAGCAGCUGAUGUGCCUGGCCAGGAGCGUGCUG
AGCAAGGCCAAGAUCCUGCUGCUGGACGAGCCCAGCGCCCACCUGGACC
CCGUGACCUACCAGAUCAUCAGGAGGACCCUGAAGCAGGCCUUCGCCGA
CUGCACCGUGAUCCUGUGCGAGCACAGGAUCGAGGCCAUGCUGGAGUGC
CAGCAGUUCCUGGUGAUCGAGGAGAACAAGGUGCGGCAGUACGACAGCA
UCCAGAAGCUGCUGAACGAGAGGAGCCUGUUCCGGCAGGCCAUCAGCCC
CAGCGACAGGGUGAAGCUGUUCCCCCACCGGAACAGCAGCAAGUGCAAG
AGCAAGCCCCAGAUCGCCGCCCUGAAGGAGGAGACAGAGGAGGAGGUGC
AGGACACCAGGCUGUAGAUAAGUGAACUCGAGCUAGUGACUGACUAGGA
UCUGGUUACCACUAAACCAGCCUCAAGAACACCCGAAUGGAGUCUCUAA
GCUACAUAAUACCAACUUACACUUACAAAAUGUUGUCCCCCAAAAUGUA
GCCAUUCGUAUCUGCUCCUAAUAAAAAGAAAGUUUCUUCACAUUCUAG
(mARM1948)
SEQ ID NO: 57
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCAUGCAGAGGUCCCCCU
UGGAAAAAGCCUCCGUGGUGUCUAAAUUGUUCUUCUCCUGGACAAGGCC
CAUAUUGAGGAAAGGAUACAGGCAGAGGUUGGAAUUGUCCGACAUAUAC
CAGAUACCCUCCGUGGACUCCGCCGACAACUUGUCCGAAAAAUUGGAAA
GGGAAUGGGAUAGGGAAUUGGCCUCCAAAAAAAACCCCAAAUUGAUAAA
CGCCUUGAGGAGGUGCUUCUUCUGGAGGUUCAUGUUCUACGGAAUAUUC
UUGUACUUGGGAGAAGUGACAAAAGCCGUGCAGCCCUUGUUGUUGGGAA
GGAUAAUAGCCUCCUACGACCCCGACAACAAAGAAGAAAGGUCCAUAGC
CAUAUACUUGGGAAUAGGAUUGUGCUUGUUGUUCAUAGUGAGGACAUUG
UUGUUGCACCCCGCCAUAUUCGGAUUGCACCACAUAGGAAUGCAGAUGA
GGAUAGCCAUGUUCUCCUUGAUAUACAAAAAAACAUUGAAAUUGUCCUC
CAGGGUGUUGGACAAAAUAUCCAUAGGACAGUUGGUGUCCUUGUUGUCC
AACAACUUGAACAAAUUCGACGAAGGAUUGGCCUUGGCCCACUUCGUGU
GGAUAGCCCCCUUGCAGGUGGCCUUGUUGAUGGGAUUGAUAUGGGAAUU
GUUGCAGGCCUCCGCCUUCUGCGGAUUGGGAUUCUUGAUAGUGUUGGCC
UUGUUCCAGGCCGGAUUGGGAAGGAUGAUGAUGAAAUAUAGGGACCAGA
GGGCCGGAAAAAUAUCCGAAAGGUUGGUGAUAACAUCCGAAAUGAUAGA
AAACAUACAGUCCGUGAAAGCCUACUGCUGGGAAGAAGCCAUGGAAAAA
AUGAUAGAAAACUUGAGGCAGACAGAAUUGAAAUUGACAAGGAAAGCCG
CCUACGUGAGGUACUUCAACUCCUCCGCCUUCUUCUUCUCCGGAUUCUU
CGUGGUGUUCUUGUCCGUGUUGCCCUACGCCUUGAUAAAAGGAAUAAUA
UUGAGGAAAAUAUUCACAACAAUAUCCUUCUGCAUAGUGUUGAGGAUGG
CCGUGACAAGGCAGUUCCCCUGGGCCGUGCAGACAUGGUAUGACUCCUU
GGGAGCCAUAAACAAAAUACAGGACUUCUUGCAGAAACAGGAAUACAAA
ACAUUGGAAUACAACUUGACAACAACAGAAGUGGUGAUGGAAAACGUGA
CAGCCUUCUGGGAAGAAGGAUUCGGAGAAUUGUUCGAAAAAGCCAAACA
GAACAACAACAACAGGAAAACAUCCAACGGAGACGACUCCUUGUUCUUC
UCCAACUUCUCCUUGUUGGGAACACCCGUGUUGAAAGACAUAAACUUCA
AAAUAGAAAGGGGACAGUUGUUGGCCGUGGCCGGAUCCACAGGAGCCGG
AAAAACAUCCUUGUUGAUGGUGAUAAUGGGAGAAUUGGAACCCUCCGAA
GGAAAAAUAAAACACUCCGGAAGGAUAUCCUUCUGCUCCCAGUUCUCCU
GGAUAAUGCCCGGAACAAUAAAAGAAAACAUAAUAUUCGGAGUGUCCUA
CGACGAAUACAGGUACAGGUCCGUGAUAAAAGCCUGCCAGUUGGAAGAA
GACAUAUCCAAAUUCGCCGAAAAAGACAACAUAGUGUUGGGAGAAGGAG
GAAUAACAUUGUCCGGAGGACAGAGGGCCAGGAUAUCCUUGGCCAGGGC
CGUGUACAAAGACGCCGACUUGUACUUGUUGGACUCCCCCUUCGGAUAC
UUGGACGUGUUGACAGAAAAAGAAAUAUUCGAAUCCUGCGUGUGCAAAU
UGAUGGCCAACAAAACAAGGAUAUUGGUGACAUCCAAAAUGGAACACUU
GAAAAAAGCCGACAAAAUAUUGAUAUUGCACGAAGGAUCCUCCUACUUC
UACGGAACAUUCUCCGAAUUGCAGAACUUGCAGCCCGACUUCUCCUCCA
AAUUGAUGGGAUGCGACUCCUUUGACCAGUUCUCCGCCGAAAGGAGGAA
CUCCAUAUUGACAGAAACAUUGCACAGGUUCUCCUUGGAAGGAGACGCC
CCCGUGUCCUGGACAGAAACAAAAAAACAGUCCUUCAAACAGACAGGAG
AAUUCGGAGAAAAAAGGAAAAACUCCAUAUUGAACCCCAUAAACUCCAU
AAGGAAAUUCUCCAUAGUGCAGAAAACACCCUUGCAGAUGAACGGAAUA
GAAGAAGACUCCGACGAACCCUUGGAAAGGAGGUUGUCCUUGGUGCCCG
ACUCCGAACAGGGAGAAGCCAUAUUGCCCAGGAUAUCCGUGAUAUCCAC
AGGACCCACAUUGCAGGCCAGGAGGAGGCAGUCCGUGUUGAACUUGAUG
ACACACUCCGUGAACCAGGGACAGAACAUACACAGGAAAACAACAGCCU
CCACAAGGAAAGUGUCCUUGGCCCCCCAGGCCAACUUGACAGAAUUGGA
CAUAUACUCCAGGAGGUUGUCCCAGGAAACAGGAUUGGAAAUAUCCGAA
GAAAUAAACGAAGAAGACUUGAAAGAAUGCUUCUUCGAUGACAUGGAAU
CCAUACCCGCCGUGACAACAUGGAACACAUACUUGAGGUACAUAACAGU
GCAUAAAUCCUUGAUAUUCGUGUUGAUAUGGUGCUUGGUGAUAUUCUUG
GCUGAAGUGGCCGCCUCCUUGGUGGUGUUGUGGUUGUUGGGAAACACAC
CCUUGCAGGACAAAGGAAACUCCACACACUCCUCCAACAACUCCUACGC
CGUGAUAAUAACAUCCACAUCCUCCUACUACGUGUUCUACAUAUACGUG
GGAGUGGCCGACACAUUGUUGGCCAUGGGAUUCUUCAGGGGAUUGCCCU
UGGUGCACACAUUGAUAACAGUGUCCAAAAUAUUGCACCACAAAAUGUU
GCACUCCGUGUUGCAGGCCCCCAUGUCCACAUUGAACACAUUGAAAGCC
GGAGGAAUAUUGAACAGGUUCUCCAAAGACAUAGCCAUAUUGGACGACU
UGUUGCCCUUGACAAUAUUCGACUUCAUACAGUUGUUGUUGAUAGUGAU
AGGAGCCAUAGCCGUGGUGGCCGUGUUGCAGCCCUACAUAUUCGUGGCC
ACAGUGCCCGUGAUAGUGGCCUUCAUAAUGUUGAGGGCCUACUUCUUGC
AGACAUCCCAGCAGUUGAAACAGUUGGAAUCCGAAGGAAGGUCCCCCAU
AUUCACACACUUGGUGACAUCCUUGAAAGGAUUGUGGACAUUGAGGGCC
UUCGGAAGGCAGCCCUACUUCGAAACAUUGUUCCACAAAGCCUUGAACU
UGCACACAGCCAACUGGUUCUUGUACUUGUCCACAUUGAGGUGGUUCCA
GAUGAGGAUAGAAAUGAUAUUCGUGAUAUUCUUCAUAGCCGUGACAUUC
AUAUCCAUAUUGACAACAGGAGAAGGAGAAGGAAGGGUGGGAAUAAUAU
UGACAUUGGCCAUGAACAUAAUGUCCACAUUGCAGUGGGCCGUGAACUC
CUCCAUAGACGUGGACUCCUUGAUGAGGUCCGUGUCCAGGGUGUUCAAA
UUCAUAGACAUGCCCACAGAAGGAAAACCCACAAAAUCCACAAAACCCU
ACAAAAACGGACAGUUGUCCAAAGUGAUGAUAAUAGAAAACUCCCACGU
GAAAAAAGACGACAUAUGGCCCUCCGGAGGACAGAUGACAGUGAAAGAC
UUGACAGCCAAAUACACAGAAGGAGGAAACGCCAUAUUGGAAAACAUAU
CCUUCUCCAUAUCCCCCGGACAGAGGGUGGGAUUGUUGGGAAGGACAGG
AUCCGGAAAAUCCACAUUGUUGUCCGCCUUCUUGAGGUUGUUGAACACA
GAAGGAGAAAUACAGAUAGACGGAGUGUCCUGGGACUCCAUAACAUUGC
AGCAGUGGAGGAAAGCCUUCGGAGUGAUACCCCAGAAAGUGUUCAUAUU
CUCCGGAACAUUCAGGAAAAACUUGGACCCCUACGAACAGUGGUCCGAC
CAGGAAAUAUGGAAAGUGGCCGACGAAGUGGGAUUGAGGUCCGUGAUAG
AACAGUUCCCCGGAAAAUUGGACUUCGUGUUGGUGGACGGAGGAUGCGU
GUUGUCCCACGGACACAAACAGUUGAUGUGCUUGGCCAGGUCCGUGUUG
UCCAAAGCCAAAAUAUUGUUGUUGGACGAACCCUCCGCCCACUUGGACC
CCGUGACAUACCAGAUAAUAAGGAGGACAUUGAAACAGGCCUUCGCCGA
CUGCACAGUGAUAUUGUGCGAACACAGGAUAGAAGCCAUGUUGGAAUGC
CAGCAGUUCUUGGUGAUAGAAGAAAACAAAGUGAGGCAGUACGACUCCA
UACAGAAAUUGUUGAACGAAAGGUCCUUGUUCAGGCAGGCCAUAUCCCC
CUCCGACAGGGUGAAAUUGUUCCCCCACAGGAACUCCUCCAAAUGCAAA
UCCAAACCCCAGAUAGCCGCCUUGAAAGAAGAAACAGAAGAAGAAGUGC
AGGACACAAGGUUGUAGAUAAGUGAACUCGAGCUAGUGACUGACUAGGA
UCUGGUUACCACUAAACCAGCCUCAAGAACACCCGAAUGGAGUCUCUAA
GCUACAUAAUACCAACUUACACUUACAAAAUGUUGUCCCCCAAAAUGUA
GCCAUUCGUAUCUGCUCCUAAUAAAAAGAAAGUUUCUUCACAUUCUAG
(mARM2047)
SEQ ID NO: 58
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCAAUCAACAG
CAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2048)
SEQ ID NO: 59
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUCAGCAAGCUGUUCUUCAGCUGGACCAG
ACCAAUCCUGAGGAAAGGAUACAGACAGCGCCUGGAACUGAGCGACAUA
UACCAAAUCCCCAGCGUGGACAGCGCCGACAACCUAAGCGAGAAGCUGG
AAAGAGAAUGGGAUAGGGAGCUGGCCAGCAAGAAGAACCCCAAGCUCAU
CAACGCCCUGCGGCGAUGCUUCUUCUGGAGGUUCAUGUUCUACGGAAUC
UUCCUGUACCUGGGGGAGGUCACCAAGGCAGUACAGCCCCUCCUGCUGG
GCAGAAUCAUAGCCAGCUACGACCCCGACAACAAGGAGGAACGCAGCAU
CGCGAUCUACCUGGGCAUCGGCCUGUGCCUGCUGUUCAUCGUGAGGACA
CUGCUCCUACACCCCGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUAGACAAGAUCAGCAUCGGACAGCUGGUGAGCCUGCUG
AGCAACAACCUGAACAAGUUCGACGAAGGACUGGCACUGGCACACUUCG
UGUGGAUCGCCCCACUGCAAGUGGCACUCCUGAUGGGGCUGAUCUGGGA
GCUGCUGCAGGCGAGCGCCUUCUGCGGACUGGGCUUCCUGAUAGUCCUG
GCCCUGUUCCAGGCCGGGCUAGGGAGAAUGAUGAUGAAGUACAGAGACC
AGAGGGCCGGGAAGAUCAGCGAGAGACUCGUGAUCACCAGCGAGAUGAU
CGAGAACAUCCAGAGCGUGAAGGCAUACUGCUGGGAAGAGGCAAUGGAG
AAGAUGAUCGAGAACCUGAGACAGACAGAGCUGAAGCUGACCCGGAAGG
CAGCCUACGUGAGAUACUUCAACAGCAGCGCCUUCUUCUUCAGCGGGUU
CUUCGUGGUCUUCCUGAGCGUGCUGCCCUACGCACUAAUCAAGGGAAUC
AUCCUGCGGAAGAUCUUCACAACCAUCAGCUUCUGCAUCGUGCUGCGCA
UGGCGGUCACCCGGCAGUUCCCCUGGGCCGUACAGACAUGGUACGACAG
CCUGGGAGCCAUCAACAAGAUACAGGACUUCCUGCAGAAGCAAGAGUAC
AAGACACUGGAGUACAACCUGACGACCACAGAAGUAGUGAUGGAGAACG
UAACCGCCUUCUGGGAGGAGGGAUUCGGGGAGCUGUUCGAGAAAGCAAA
GCAGAACAACAACAACCGGAAGACCAGCAACGGCGACGACAGCCUCUUC
UUCAGCAACUUCAGCCUGCUGGGCACCCCCGUCCUGAAGGACAUCAACU
UCAAGAUAGAGAGGGGACAGCUGCUGGCGGUGGCCGGAAGCACCGGAGC
AGGCAAGACCAGCCUGCUAAUGGUGAUCAUGGGAGAACUGGAGCCCAGC
GAGGGCAAGAUCAAGCACAGCGGAAGGAUCAGCUUCUGCAGCCAGUUCA
GCUGGAUCAUGCCCGGCACCAUCAAGGAGAACAUCAUCUUCGGCGUGAG
CUACGACGAGUACCGCUACAGAAGCGUCAUCAAGGCAUGCCAACUAGAA
GAGGACAUCAGCAAGUUCGCAGAGAAGGACAACAUAGUGCUGGGAGAAG
GCGGAAUCACACUGAGCGGAGGCCAACGAGCAAGAAUCAGCCUGGCAAG
AGCAGUAUACAAGGACGCCGACCUGUACCUGCUGGACAGCCCCUUCGGA
UACCUAGACGUGCUGACCGAGAAGGAGAUAUUCGAAAGCUGCGUCUGCA
AGCUGAUGGCCAACAAGACCAGGAUCCUGGUCACCAGCAAGAUGGAACA
CCUGAAGAAAGCCGACAAGAUCCUGAUCCUGCACGAAGGCAGCAGCUAC
UUCUACGGGACAUUCAGCGAACUCCAGAACCUACAGCCAGACUUCAGCA
GCAAGCUCAUGGGAUGCGACAGCUUCGACCAGUUCAGCGCAGAGAGACG
GAACAGCAUCCUAACCGAGACACUGCACAGGUUCAGCCUGGAAGGAGAC
GCCCCCGUCAGCUGGACAGAGACGAAGAAACAGAGCUUCAAACAGACCG
GAGAGUUCGGGGAGAAACGCAAGAACAGCAUCCUCAACCCAAUCAACAG
CAUACGAAAGUUCAGCAUCGUGCAGAAGACCCCACUGCAGAUGAACGGC
AUCGAAGAGGACAGCGACGAGCCCCUGGAGAGAAGGCUGAGCCUGGUAC
CAGACAGCGAGCAGGGAGAGGCGAUACUGCCCCGCAUCAGCGUGAUCAG
CACCGGCCCCACGCUGCAGGCACGAAGGCGCCAGAGCGUCCUGAACCUG
AUGACACACAGCGUGAACCAAGGCCAGAACAUCCACCGAAAGACAACCG
CAAGCACAAGGAAGGUGAGCCUGGCCCCACAGGCAAACCUGACCGAACU
GGACAUCUACAGCAGAAGGCUGAGCCAGGAGACCGGCCUGGAGAUCAGC
GAAGAGAUCAACGAAGAGGACCUGAAGGAGUGCUUCUUCGACGACAUGG
AGAGCAUACCAGCAGUGACCACAUGGAACACAUACCUGAGGUACAUCAC
CGUCCACAAGAGCCUGAUCUUCGUGCUAAUCUGGUGCCUGGUGAUCUUC
CUGGCAGAGGUGGCCGCCAGCCUGGUGGUGCUGUGGCUCCUGGGAAACA
CCCCACUGCAAGACAAAGGGAACAGCACCCACAGCAGGAACAACAGCUA
CGCAGUGAUCAUCACCAGCACCAGCAGCUACUACGUGUUCUACAUCUAC
GUGGGAGUAGCCGACACCCUGCUGGCCAUGGGAUUCUUCAGAGGCCUAC
CACUGGUGCACACCCUAAUCACAGUGAGCAAGAUCCUGCACCACAAGAU
GCUGCACAGCGUGCUGCAAGCACCCAUGAGCACCCUCAACACGCUGAAG
GCAGGCGGGAUCCUGAACAGGUUCAGCAAGGACAUAGCCAUCCUGGACG
ACCUGCUGCCCCUGACCAUCUUCGACUUCAUCCAGCUGCUGCUGAUCGU
GAUCGGAGCCAUAGCAGUGGUCGCAGUGCUGCAACCCUACAUCUUCGUG
GCAACAGUGCCAGUGAUAGUGGCCUUCAUCAUGCUGAGAGCAUACUUCC
UCCAAACCAGCCAGCAACUCAAGCAGCUGGAAAGCGAAGGCAGGAGCCC
AAUCUUCACCCACCUGGUGACAAGCCUGAAGGGACUCUGGACACUGAGG
GCCUUCGGACGGCAGCCCUACUUCGAAACCCUGUUCCACAAAGCCCUGA
ACCUGCACACCGCCAACUGGUUCCUGUACCUGAGCACACUGCGCUGGUU
CCAAAUGAGAAUAGAAAUGAUCUUCGUCAUCUUCUUCAUCGCCGUGACC
UUCAUCAGCAUCCUGACAACAGGAGAAGGAGAAGGAAGAGUGGGCAUCA
UCCUGACCCUGGCCAUGAACAUCAUGAGCACACUGCAGUGGGCCGUGAA
CAGCAGCAUAGACGUGGACAGCCUGAUGCGAAGCGUGAGCCGAGUCUUC
AAGUUCAUCGACAUGCCCACCGAGGGCAAGCCCACCAAGAGCACCAAGC
CCUACAAGAACGGCCAACUCAGCAAGGUGAUGAUCAUCGAGAACAGCCA
CGUGAAGAAAGACGACAUCUGGCCCAGCGGGGGCCAAAUGACCGUCAAA
GACCUCACAGCCAAGUACACAGAAGGCGGAAACGCCAUCCUGGAGAACA
UCAGCUUCAGCAUCAGCCCCGGCCAGAGGGUGGGCCUCCUGGGAAGAAC
CGGAAGCGGCAAGAGCACCCUGCUGAGCGCCUUCCUGAGACUACUGAAC
ACCGAAGGCGAGAUCCAGAUCGACGGCGUGAGCUGGGACAGCAUCACCC
UGCAACAGUGGAGGAAGGCCUUCGGCGUGAUACCACAGAAGGUGUUCAU
CUUCAGCGGAACCUUCAGGAAGAACCUGGACCCCUACGAACAGUGGAGC
GACCAGGAGAUCUGGAAGGUGGCAGACGAGGUGGGGCUCAGAAGCGUGA
UAGAACAGUUCCCCGGGAAGCUGGACUUCGUCCUGGUGGACGGGGGCUG
CGUCCUAAGCCACGGCCACAAGCAGCUGAUGUGCCUGGCCAGAAGCGUG
CUCAGCAAGGCGAAGAUCCUGCUGCUGGACGAACCCAGCGCCCACCUGG
ACCCAGUAACAUACCAGAUCAUCCGGAGAACCCUGAAGCAGGCAUUCGC
CGACUGCACAGUAAUCCUCUGCGAACACAGGAUAGAAGCAAUGCUGGAA
UGCCAACAGUUCCUGGUCAUCGAAGAGAACAAGGUGCGGCAGUACGACA
GCAUCCAGAAGCUGCUGAACGAGAGGAGCCUCUUCCGGCAAGCCAUCAG
CCCCAGCGACAGGGUGAAGCUGUUCCCCCACCGGAACAGCAGCAAGUGC
AAGAGCAAGCCCCAGAUCGCCGCCCUGAAGGAAGAGACCGAGGAAGAGG
UGCAGGACACCAGGCUGUGAAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
SEQ ID NO: 60
(mARM2049)
AUUAUUACAUCAAAACAAAAAGCCGCCACCAUGCAGAGGAGCCCCCUGG
AGAAGGCUAGCGUGGUGAGCAAGCUGUUCUUCAGCUGGACCAGACCAAU
CCUGAGGAAGGGCUACAGACAGCGCCUGGAGCUGAGCGACAUCUACCAG
AUCCCCAGCGUGGACAGCGCCGACAACCUGAGCGAGAAGCUGGAGAGAG
AGUGGGACAGAGAGCUGGCCAGCAAGAAGAACCCCAAGCUGAUCAACGC
CCUGCGGAGGUGCUUCUUCUGGAGAUUCAUGUUCUACGGAAUCUUCCUG
UACCUGGGGGAGGUGACCAAGGCCGUGCAGCCCCUGCUGCUGGGAAGAA
UCAUCGCCAGCUACGACCCCGACAACAAGGAGGAGCGCAGCAUCGCCAU
CUACCUGGGCAUCGGCCUGUGCCUGCUGUUCAUCGUGAGGACCCUGCUG
CUGCACCCAGCCAUCUUCGGCCUGCACCACAUCGGAAUGCAGAUGAGAA
UCGCCAUGUUCAGCCUGAUCUACAAGAAGACCCUGAAGCUGAGCAGCAG
GGUGCUGGACAAGAUCAGCAUCGGACAGCUGGUGAGCCUGCUGAGCAAC
AACCUGAACAAGUUCGACGAGGGACUGGCCCUGGCCCACUUCGUGUGGA
UCGCCCCACUGCAGGUGGCCCUGCUGAUGGGGCUGAUCUGGGAGCUGCU
GCAGGCCAGCGCCUUCUGCGGCCUGGGCUUCCUGAUCGUGCUGGCCCUG
UUCCAGGCCGGCCUGGGCAGAAUGAUGAUGAAGUACAGAGACCAGAGAG
CCGGCAAGAUCAGCGAGAGACUGGUGAUCACCAGCGAGAUGAUCGAGAA
CAUCCAGAGCGUGAAGGCAUACUGCUGGGAGGAGGCCAUGGAGAAGAUG
AUCGAGAACCUGAGACAGACCGAGCUGAAGCUGACCCGGAAGGCCGCCU
ACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUG
CGGAAGAUCUUCACCACCAUCAGCUUCUGCAUCGUGCUGCGCAUGGCCG
UGACCCGGCAGUUCCCCUGGGCCGUGCAGACCUGGUACGACAGCCUGGG
AGCCAUCAACAAGAUCCAGGACUUCCUGCAGAAGCAGGAGUACAAGACC
CUGGAGUACAACCUGACCACCACCGAGGUGGUGAUGGAGAACGUGACCG
CCUUCUGGGAGGAGGGAUUCGGCGAGCUGUUCGAGAAGGCCAAGCAGAA
CAACAACAACAGAAAGACCAGCAACGGCGACGACAGCCUGUUCUUCAGC
AACUUCAGCCUGCUGGGCACCCCCGUGCUGAAGGACAUCAACUUCAAGA
UCGAGAGAGGACAGCUGCUGGCCGUGGCCGGAAGCACCGGAGCCGGCAA
GACCAGCCUGCUGAUGGUGAUCAUGGGAGAGCUGGAGCCCAGCGAGGGC
AAGAUCAAGCACAGCGGAAGAAUCAGCUUCUGCAGCCAGUUCAGCUGGA
UCAUGCCCGGCACCAUCAAGGAGAACAUCAUCUUCGGCGUGAGCUACGA
CGAGUACAGAUACAGAAGCGUGAUCAAGGCCUGCCAGCUGGAGGAGGAC
AUCAGCAAGUUCGCAGAGAAGGACAACAUCGUGCUGGGAGAGGGCGGCA
UCACCCUGAGCGGAGGCCAGAGGGCCAGAAUCAGCCUGGCAAGAGCAGU
GUACAAGGACGCCGACCUGUACCUGCUGGACAGCCCCUUCGGAUACCUG
GACGUGCUGACCGAGAAGGAGAUCUUCGAGAGCUGCGUGUGCAAGCUGA
UGGCCAACAAGACCAGGAUCCUGGUGACCAGCAAGAUGGAGCACCUGAA
GAAGGCCGACAAGAUCCUGAUCCUGCACGAGGGCAGCAGCUACUUCUAC
GGGACCUUCAGCGAGCUGCAGAACCUGCAGCCAGACUUCAGCAGCAAGC
UGAUGGGCUGCGACAGCUUCGACCAGUUCAGCGCCGAGAGAAGAAACAG
CAUCCUGACCGAGACCCUGCACAGGUUCAGCCUGGAGGGCGACGCCCCC
GUGAGCUGGACCGAGACCAAGAAGCAGAGCUUCAAGCAGACCGGAGAGU
UCGGCGAGAAGAGGAAGAACAGCAUCCUGAACCCAAUCAACAGCAUCAG
GAAGUUCAGCAUCGUGCAGAAGACCCCACUGCAGAUGAACGGCAUCGAG
GAGGACAGCGACGAGCCCCUGGAGAGAAGGCUGAGCCUGGUGCCAGACA
GCGAGCAGGGCGAGGCCAUCCUGCCCCGCAUCAGCGUGAUCAGCACCGG
CCCCACCCUGCAGGCCAGGAGGAGGCAGAGCGUGCUGAACCUGAUGACC
CACAGCGUGAACCAGGGCCAGAACAUCCACAGGAAGACCACCGCCAGCA
CCAGGAAGGUGAGCCUGGCCCCACAGGCCAACCUGACCGAGCUGGACAU
CUACAGCAGAAGGCUGAGCCAGGAGACCGGCCUGGAGAUCAGCGAGGAG
AUCAACGAGGAGGACCUGAAGGAGUGCUUCUUCGACGACAUGGAGAGCA
UCCCAGCCGUGACCACCUGGAACACCUACCUGAGGUACAUCACCGUGCA
CAAGAGCCUGAUCUUCGUGCUGAUCUGGUGCCUGGUGAUCUUCCUGGCC
GAGGUGGCCGCCAGCCUGGUGGUGCUGUGGCUGCUGGGCAACACCCCAC
UGCAGGACAAGGGCAACAGCACCCACAGCAGAAACAACAGCUACGCCGU
GAUCAUCACCAGCACCAGCAGCUACUACGUGUUCUACAUCUACGUGGGA
GUGGCCGACACCCUGCUGGCCAUGGGCUUCUUCAGAGGCCUGCCACUGG
UGCACACCCUGAUCACCGUGAGCAAGAUCCUGCACCACAAGAUGCUGCA
CAGCGUGCUGCAGGCCCCCAUGAGCACCCUGAACACCCUGAAGGCCGGC
GGGAUCCUGAACAGAUUCAGCAAGGACAUCGCCAUCCUGGACGACCUGC
UGCCCCUGACCAUCUUCGACUUCAUCCAGCUGCUGCUGAUCGUGAUCGG
AGCCAUCGCCGUGGUGGCCGUGCUGCAGCCCUACAUCUUCGUGGCCACC
GUGCCAGUGAUCGUGGCCUUCAUCAUGCUGAGAGCCUACUUCCUGCAGA
CCAGCCAGCAGCUGAAGCAGCUGGAGAGCGAGGGCAGGAGCCCAAUCUU
CACCCACCUGGUGACCAGCCUGAAGGGACUGUGGACCCUGAGGGCCUUC
GGCCGGCAGCCCUACUUCGAGACCCUGUUCCACAAGGCCCUGAACCUGC
ACACCGCCAACUGGUUCCUGUACCUGAGCACCCUGCGCUGGUUCCAGAU
GAGAAUCGAGAUGAUCUUCGUGAUCUUCUUCAUCGCCGUGACCUUCAUC
AGCAUCCUGACCACCGGCGAGGGAGAGGGAAGAGUGGGCAUCAUCCUGA
CCCUGGCCAUGAACAUCAUGAGCACCCUGCAGUGGGCCGUGAACAGCAG
CAUCGACGUGGACAGCCUGAUGAGGAGCGUGAGCAGGGUGUUCAAGUUC
AUCGACAUGCCAACCGAGGGCAAGCCCACCAAGAGCACCAAGCCAUACA
AGAACGGCCAGCUGAGCAAGGUGAUGAUCAUCGAGAACAGCCACGUGAA
GAAGGACGACAUCUGGCCCAGCGGCGGCCAGAUGACCGUGAAGGACCUG
ACCGCCAAGUACACCGAGGGCGGCAACGCCAUCCUGGAGAACAUCAGCU
UCAGCAUCAGCCCCGGCCAGAGGGUGGGCCUGCUGGGAAGAACCGGCAG
CGGCAAGAGCACCCUGCUGAGCGCCUUCCUGAGACUGCUGAACACCGAG
GGCGAGAUCCAGAUCGACGGCGUGAGCUGGGACAGCAUCACCCUGCAGC
AGUGGAGGAAGGCCUUCGGCGUGAUCCCACAGAAGGUGUUCAUCUUCAG
CGGAACCUUCAGAAAGAACCUGGACCCCUACGAGCAGUGGAGCGACCAG
GAGAUCUGGAAGGUGGCCGACGAGGUGGGCCUGAGAAGCGUGAUCGAGC
AGUUCCCCGGCAAGCUGGACUUCGUGCUGGUGGACGGGGGCUGCGUGCU
GAGCCACGGCCACAAGCAGCUGAUGUGCCUGGCCAGAAGCGUGCUGAGC
AAGGCCAAGAUCCUGCUGCUGGACGAGCCCAGCGCCCACCUGGACCCAG
UGACCUACCAGAUCAUCAGAAGAACCCUGAAGCAGGCCUUCGCCGACUG
CACCGUGAUCCUGUGCGAGCACAGGAUCGAGGCCAUGCUGGAGUGCCAG
CAGUUCCUGGUGAUCGAGGAGAACAAGGUGCGGCAGUACGACAGCAUCC
AGAAGCUGCUGAACGAGAGGAGCCUGUUCCGGCAGGCCAUCAGCCCCAG
CGACAGGGUGAAGCUGUUCCCCCACCGGAACAGCAGCAAGUGCAAGAGC
AAGCCCCAGAUCGCCGCCCUGAAGGAGGAGACCGAGGAGGAGGUGCAGG
ACACCAGGCUGUAGAUAAGUGAACUCGAGGCUGGAGCCUCGGUAGCCGU
UCCUCCUGCCCGCUGGGCCUCCCAACGGGCCCUCCUCCCCUCCUUGCAC
CGGCCCUUCCUGGUCUUUGAAUAAAGUCUGAGUGGGCAUCUAG
(mARM2088)
SEQ ID NO: 61
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCCAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCCGCGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGCGCGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGCGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGCGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCCAUCAACAG
CAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGCGCGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCCCAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCCGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUUUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2089)
SEQ ID NO: 62
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGCGCAGCC
CCCUGGAGAAGGCCAGCGUGGUGAGCAAGCUGUUCUUCAGCUGGACCCG
CCCCAUCCUGCGCAAGGGCUACAGACAGCGCCUGGAGCUGAGCGACAUC
UACCAGAUCCCUUCUGUGGACAGCGCCGACAACCUGAGCGAGAAGCUGG
AGCGCGAGUGGGACCGCGAGCUGGCCAGCAAGAAGAACCCUAAGCUGAU
CAACGCCCUGCGCCGCUGCUUCUUCUGGCGCUUCAUGUUCUACGGCAUC
UUCCUGUACCUGGGGGAGGUGACCAAGGCCGUGCAGCCCCUGCUGCUGG
GCCGCAUCAUCGCCAGCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCCGCCAUCUUCGGCCUGCACCACAUCGGCAUGCAGA
UGCGCAUCGCCAUGUUCAGCCUGAUCUACAAGAAGACCCUGAAGCUGUC
AAGCAGGGUGCUGGACAAGAUCAGCAUCGGCCAGCUGGUGAGCCUGCUG
AGCAACAACCUGAACAAGUUCGACGAGGGCCUGGCCCUGGCCCACUUCG
UGUGGAUCGCUCCCCUGCAGGUGGCCCUGCUGAUGGGCCUGAUCUGGGA
GCUGCUGCAGGCCAGCGCCUUCUGCGGCCUGGGCUUCCUGAUCGUGCUG
GCCCUGUUCCAGGCCGGCCUGGGCCGCAUGAUGAUGAAGUACAGAGACC
AGCGCGCCGGCAAGAUCAGCGAGCGCCUGGUGAUCACCAGCGAGAUGAU
CGAGAACAUCCAGAGCGUGAAGGCCUACUGCUGGGAGGAGGCCAUGGAG
AAGAUGAUCGAGAACCUGCGCCAGACCGAGCUGAAGCUGACCCGCAAGG
CCGCCUACGUGCGCUACUUCAACAGCAGCGCCUUCUUCUUCUCAGGCUU
CUUCGUGGUGUUCCUGAGCGUGCUGCCCUACGCCCUGAUCAAGGGCAUC
AUCCUGCGCAAGAUCUUCACCACCAUCAGCUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGCUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGCAGCACCGGCGC
CGGCAAGACCUCACUGCUGAUGGUGAUCAUGGGCGAGCUGGAGCCUAGC
GAGGGCAAGAUCAAGCACAGCGGCCGCAUCUCAUUCUGCAGCCAGUUCA
GCUGGAUCAUGCCCGGCACCAUCAAGGAGAACAUCAUCUUCGGUGUGAG
CUACGACGAGUACCGCUACCGCAGCGUGAUCAAGGCCUGCCAGCUGGAG
GAGGACAUCAGCAAGUUCGCCGAGAAGGACAACAUCGUGCUGGGAGAGG
GUGGCAUCACCCUGAGCGGCGGCCAGAGGGCCCGCAUCAGCCUGGCCCG
CGCCGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCCGCAUCCUGGUGACCUCUAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGCGCCG
CAACAGCAUCCUGACCGAGACCCUGCACCGCUUCAGCCUGGAGGGCGAC
GCCCCCGUGAGCUGGACCGAGACCAAGAAGCAGUCUUUCAAGCAGACCG
GCGAGUUCGGCGAGAAGCGCAAGAACAGCAUCCUGAACCCCAUCAACAG
CAUCCGCAAGUUCAGCAUCGUGCAGAAGACCCCACUGCAGAUGAACGGC
AUCGAGGAGGACAGCGACGAGCCCCUGGAGCGCCGCCUGAGCCUGGUGC
CCGACAGCGAGCAGGGCGAGGCCAUCCUGCCUCGCAUCAGCGUGAUCAG
CACCGGCCCCACCCUGCAGGCCCGCCGCCGCCAGAGCGUGCUGAACCUG
AUGACCCACAGCGUGAACCAGGGCCAGAACAUCCACAGGAAGACCACCG
CCAGCACCAGGAAAGUGAGCCUGGCCCCUCAGGCCAACCUGACCGAGCU
GGACAUCUACAGCCGCAGGCUGAGCCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCUCUGCAGGACAAGGGCAACAGCACCCACAGCCGCAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCCGCGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGUGGCAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCCGUGAUCGUGGCCUUCAUCAUGCUGCGCGCCUACUUCC
UGCAGACCAGCCAGCAGCUGAAGCAGCUGGAGAGCGAGGGCCGCAGCCC
CAUCUUCACCCACCUGGUGACCAGCCUGAAGGGCCUGUGGACCCUGCGC
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGCGAGGGACGCGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCCACCGAGGGCAAGCCUACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCUCAAUCAGCCCCGGCCAGCGCGUGGGCCUGCUGGGCCGCAC
CGGCUCAGGCAAGAGCACCCUGCUGAGCGCCUUCCUGCGCCUGCUGAAC
ACCGAGGGCGAGAUCCAGAUCGACGGCGUGAGCUGGGACAGCAUCACCC
UGCAGCAGUGGCGCAAGGCCUUCGGCGUGAUCCCCCAGAAGGUGUUCAU
CUUCUCUGGCACCUUCCGCAAGAACCUGGACCCCUACGAGCAGUGGAGC
GACCAGGAGAUCUGGAAGGUGGCCGACGAGGUGGGCCUGCGCAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCCGCAGCGUG
CUGAGCAAGGCCAAGAUCCUGCUGCUGGACGAGCCCAGCGCCCACCUGG
ACCCCGUGACCUACCAGAUCAUCCGCCGCACCCUGAAGCAGGCCUUCGC
CGACUGCACCGUGAUCCUGUGCGAGCACCGCAUCGAGGCCAUGCUGGAG
UGCCAGCAGUUCCUGGUGAUCGAGGAGAACAAGGUGCGCCAGUACGACU
CCAUCCAGAAGCUGCUGAACGAGCGCAGCCUGUUCCGCCAGGCCAUCAG
CCCCAGCGACCGCGUGAAGCUUUUCCCCCACCGCAACAGCAGCAAGUGC
AAGUCUAAGCCCCAGAUCGCCGCCCUGAAGGAGGAGACCGAGGAGGAGG
UGCAGGACACCCGCCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2090)
SEQ ID NO: 63
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCCAUCCUGAGGAAGGGCUACCGCCAGCGCCUGGAGCUGAGCGACAUC
UACCAGAUCCCCAGCGUGGACAGCGCUGACAACCUGAGCGAGAAGCUGG
AGAGAGAGUGGGACCGCGAGCUGGCCAGCAAGAAGAACCCUAAGCUGAU
CAACGCCCUGCGGCGCUGCUUCUUCUGGCGCUUCAUGUUCUACGGCAUC
UUCCUGUACCUGGGCGAGGUGACCAAGGCCGUGCAGCCCCUGCUGCUGG
GCCGCAUCAUCGCCAGCUACGACCCCGACAACAAGGAGGAGCGCUCUAU
CGCCAUCUACCUGGGCAUCGGCCUGUGCCUGCUGUUCAUCGUGCGCACC
CUGCUGCUGCACCCCGCCAUCUUCGGCCUGCACCACAUCGGCAUGCAGA
UGCGCAUCGCCAUGUUCAGCCUGAUCUACAAGAAGACCCUGAAGCUGAG
CAGCCGCGUGCUGGACAAGAUCAGUAUCGGCCAGCUGGUGAGCCUGCUG
UCCAACAACCUGAACAAGUUCGACGAGGGCCUGGCCCUGGCCCACUUCG
UGUGGAUCGCCCCUCUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACCGCGACC
AGCGCGCCGGCAAGAUCAGCGAGAGACUGGUGAUCACCAGCGAGAUGAU
CGAGAACAUCCAGUCUGUGAAGGCCUACUGCUGGGAGGAGGCCAUGGAG
AAGAUGAUCGAGAACCUGCGCCAGACCGAGCUGAAGCUGACCCGGAAGG
CCGCCUACGUGCGCUACUUCAACAGCAGCGCCUUCUUCUUCUCAGGCUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGCCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGCUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGCAGCACCGGCGC
CGGCAAGACCAGCCUGCUGAUGGUGAUCAUGGGCGAGCUGGAGCCCAGC
GAGGGCAAGAUCAAGCACAGCGGACGCAUCAGCUUCUGCAGCCAGUUCU
CCUGGAUCAUGCCUGGCACCAUCAAGGAGAACAUCAUCUUCGGCGUGAG
CUACGACGAGUACCGCUACCGCAGCGUGAUCAAGGCCUGCCAGCUGGAG
GAGGACAUCAGCAAGUUCGCCGAGAAGGACAACAUCGUGCUGGGAGAGG
GCGGCAUCACCCUGAGCGGCGGCCAGAGGGCCCGCAUCAGCCUGGCAAG
AGCAGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCUGACAAGAUCCUGAUCCUGCACGAGGGCAGCAGCUAC
UUCUACGGCACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACCGCUUCAGCCUGGAGGGCGAC
GCCCCCGUGUCCUGGACCGAGACCAAGAAGCAGAGCUUCAAGCAGACCG
GCGAGUUCGGCGAGAAGCGCAAGAACUCUAUCCUGAACCCCAUCAACAG
CAUCCGCAAGUUCAGCAUCGUGCAGAAGACCCCACUGCAGAUGAACGGC
AUCGAGGAGGACAGCGACGAGCCCCUGGAGCGCCGCCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCCGCAAAGUGAGCCUGGCCCCACAGGCCAACCUGACCGAGCU
GGACAUCUACAGCCGCCGCCUGUCUCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCCGCAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCAGAGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGCAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGCCC
CAUCUUCACCCACCUGGUGACCAGCCUGAAGGGCCUGUGGACCCUGAGG
GCCUUCGGCCGGCAGCCUUACUUCGAGACCCUGUUCCACAAGGCCCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGAGAGGGCAGAGUGGGCAUCA
UCCUGACCCUGGCCAUGAACAUCAUGAGCACCCUGCAGUGGGCUGUGAA
CAGCAGCAUCGACGUGGACAGCCUGAUGCGCAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCUCAAUCAGCCCCGGCCAGCGCGUGGGCCUGCUGGGACGCAC
CGGCAGCGGCAAGAGCACCCUGCUGAGCGCCUUCCUGAGACUGCUGAAC
ACCGAGGGCGAGAUCCAGAUCGACGGCGUGAGCUGGGACUCAAUCACCC
UGCAGCAGUGGAGGAAGGCCUUCGGCGUGAUCCCCCAGAAGGUGUUCAU
CUUCAGCGGCACCUUCCGCAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCCGCUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGCCAGUACGACA
GCAUCCAGAAGCUGCUGAACGAGAGGAGCCUGUUCCGCCAGGCCAUCAG
CCCCAGCGACAGGGUGAAGCUUUUCCCCCACCGCAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2091)
SEQ ID NO: 64
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGCGCAGCC
CCCUGGAGAAGGCCAGCGUGGUGUCCAAGCUGUUCUUCAGCUGGACCCG
CCCCAUCCUGAGGAAGGGCUACCGCCAGCGCCUGGAGCUGAGCGACAUC
UACCAGAUCCCUAGCGUGGACAGCGCCGACAACCUGAGCGAGAAGCUGG
AGCGCGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GCCGCAUCAUCGCCUCCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCCGCCAUCUUCGGCCUGCACCACAUCGGCAUGCAGA
UGCGCAUCGCCAUGUUCAGCCUGAUCUACAAGAAGACCCUGAAGCUGAG
CAGCCGCGUGCUGGACAAGAUCAGCAUCGGCCAGCUGGUGAGCCUGCUG
UCCAACAACCUGAACAAGUUCGACGAGGGACUGGCCCUGGCCCACUUCG
UGUGGAUCGCCCCUCUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACCGCGACC
AGCGCGCCGGCAAGAUCAGCGAGCGCCUGGUGAUCACCAGCGAGAUGAU
CGAGAACAUCCAGAGCGUGAAGGCCUACUGCUGGGAGGAGGCCAUGGAG
AAGAUGAUCGAGAACCUGAGACAGACCGAGCUGAAGCUGACCCGCAAGG
CCGCCUACGUGAGAUACUUCAACAGCAGCGCCUUCUUCUUCAGCGGCUU
CUUCGUGGUGUUCCUGUCUGUGCUGCCCUACGCCCUGAUCAAGGGCAUC
AUCCUGCGCAAGAUCUUCACCACCAUCUCAUUCUGCAUCGUGCUGCGCA
UGGCCGUGACCCGGCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCUGUGCUGAAGGACAUCAACU
UCAAGAUCGAGCGCGGCCAGCUGCUGGCCGUGGCCGGCAGCACCGGCGC
CGGCAAGACCAGCCUGCUGAUGGUGAUCAUGGGAGAGCUGGAGCCUAGC
GAGGGCAAGAUCAAGCACAGUGGACGCAUCAGCUUCUGCAGCCAGUUCU
CCUGGAUCAUGCCUGGCACCAUCAAGGAGAACAUCAUCUUCGGCGUGUC
CUACGACGAGUACCGCUACAGAAGCGUGAUCAAGGCCUGCCAGCUGGAG
GAGGACAUCAGCAAGUUCGCCGAGAAGGACAACAUCGUGCUGGGAGAGG
GCGGCAUCACCCUGAGCGGAGGCCAGCGCGCCAGAAUCAGCCUGGCCAG
AGCCGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCAGACUUCAGCA
GCAAGCUGAUGGGCUGCGACAGCUUCGACCAGUUCAGCGCCGAGCGCCG
CAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCCAUCAACAG
CAUCAGGAAGUUCUCCAUCGUGCAGAAGACCCCACUGCAGAUGAACGGC
AUCGAGGAGGACAGCGACGAGCCCCUGGAGCGCCGCCUGAGCCUGGUGC
CAGACAGCGAGCAGGGCGAGGCCAUCCUGCCCCGCAUCAGCGUGAUCAG
CACCGGCCCCACCCUGCAGGCCCGCAGGAGGCAGAGCGUGCUGAACCUG
AUGACCCACAGCGUGAACCAGGGCCAGAACAUCCACAGGAAGACCACCG
CCUCCACCCGCAAAGUGAGCCUGGCCCCACAGGCCAACCUGACCGAGCU
GGACAUCUACAGCCGCCGCCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCCGCAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCCGCGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGUGGCAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCCGUGAUCGUGGCCUUCAUCAUGCUGAGAGCCUACUUCC
UGCAGACCAGCCAGCAGCUGAAGCAGCUGGAGAGCGAGGGCCGCAGUCC
AAUCUUCACCCACCUGGUGACCAGCCUGAAGGGCCUGUGGACCCUGCGC
GCCUUCGGCCGCCAGCCCUACUUCGAGACCCUGUUCCACAAGGCUCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGAGAGGGCAGAGUGGGCAUCA
UCCUGACCCUGGCCAUGAACAUCAUGAGCACCCUGCAGUGGGCCGUGAA
CUCCAGCAUCGACGUGGACAGCCUGAUGAGGUCUGUGAGCCGCGUGUUC
AAGUUCAUCGACAUGCCAACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCUCAAUCAGCCCCGGCCAGCGCGUGGGCCUGCUGGGACGCAC
CGGCAGCGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGCGCAAGGCCUUCGGCGUGAUCCCCCAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGCGCAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCCGUGACCUACCAGAUCAUCCGCAGAACCCUGAAGCAGGCCUUCGC
CGACUGCACCGUGAUCCUGUGCGAGCACCGCAUCGAGGCCAUGCUGGAG
UGCCAGCAGUUCCUGGUGAUCGAGGAGAACAAGGUGCGCCAGUACGACA
GCAUCCAGAAGCUGCUGAACGAGCGCAGCCUGUUCCGCCAGGCCAUCAG
CCCCAGCGACCGCGUGAAGCUUUUCCCCCACCGCAACAGCAGCAAGUGC
AAGUCUAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2092)
SEQ ID NO: 65
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGCGCAGCC
CCCUGGAGAAGGCCAGCGUGGUGAGCAAGCUGUUCUUCAGCUGGACCCG
CCCAAUCCUGCGCAAGGGCUACCGCCAGCGCCUGGAGCUGAGCGACAUC
UACCAGAUCCCCAGCGUGGACAGCGCCGACAACCUGAGCGAGAAGCUGG
AGAGAGAGUGGGACCGCGAGCUGGCCAGCAAGAAGAACCCUAAGCUGAU
CAACGCCCUGCGGCGCUGCUUCUUCUGGCGCUUCAUGUUCUACGGCAUC
UUCCUGUACCUGGGCGAGGUGACCAAGGCCGUGCAGCCCCUGCUGCUGG
GCCGCAUCAUCGCCUCCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCCGCCAUCUUCGGCCUGCACCACAUCGGCAUGCAGA
UGCGCAUCGCCAUGUUCAGCCUGAUCUACAAGAAGACCCUGAAGCUGAG
CAGCCGCGUGCUGGACAAGAUCAGCAUCGGCCAGCUGGUGAGCCUGCUG
UCCAACAACCUGAACAAGUUCGACGAGGGCCUGGCCCUGGCCCACUUCG
UGUGGAUCGCCCCACUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGCGCGCCGGCAAGAUCAGCGAGCGCCUGGUGAUCACCUCAGAGAUGAU
CGAGAACAUCCAGUCUGUGAAGGCCUACUGCUGGGAGGAGGCCAUGGAG
AAGAUGAUCGAGAACCUGCGCCAGACCGAGCUGAAGCUGACCCGCAAGG
CCGCCUACGUGCGCUACUUCAACAGCAGCGCCUUCUUCUUCUCAGGCUU
CUUCGUGGUGUUCCUGAGCGUGCUGCCCUACGCCCUGAUCAAGGGCAUC
AUCCUGCGCAAGAUCUUCACCACCAUCAGCUUCUGCAUCGUGCUGCGCA
UGGCCGUGACCCGCCAGUUCCCCUGGGCCGUGCAGACCUGGUACGACUC
UCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGCUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGCAGCACCGGCGC
CGGCAAGACCAGCCUGCUGAUGGUGAUCAUGGGCGAGCUGGAGCCUAGC
GAGGGCAAGAUCAAGCACAGCGGCAGAAUCUCAUUCUGCUCUCAGUUCA
GCUGGAUCAUGCCUGGCACCAUCAAGGAGAACAUCAUCUUCGGCGUGUC
CUACGACGAGUACCGCUACCGCAGCGUGAUCAAGGCCUGCCAGCUGGAG
GAGGACAUCAGCAAGUUCGCCGAGAAGGACAACAUCGUGCUGGGAGAGG
GCGGCAUCACCCUGAGCGGCGGCCAGCGCGCCCGCAUCAGCCUGGCCCG
CGCCGUGUACAAGGACGCCGACCUGUACCUGCUGGACUCUCCUUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCAGACUUCAGCA
GCAAGCUGAUGGGCUGCGACAGCUUCGACCAGUUCAGCGCCGAGCGCCG
CAACAGCAUCCUGACCGAGACCCUGCACCGCUUCAGCCUGGAGGGCGAC
GCCCCCGUGAGCUGGACCGAGACCAAGAAGCAGUCUUUCAAGCAGACCG
GCGAGUUCGGCGAGAAGAGGAAGAACAGCAUCCUGAACCCAAUCAACAG
CAUCCGCAAGUUCAGCAUCGUGCAGAAGACCCCACUGCAGAUGAACGGC
AUCGAGGAGGACAGCGACGAGCCUCUGGAGCGCCGCCUGUCCCUGGUGC
CCGACAGCGAGCAGGGCGAGGCCAUCCUGCCCCGCAUCAGCGUGAUCAG
CACCGGCCCCACCCUGCAGGCCCGCCGCCGCCAGAGCGUGCUGAACCUG
AUGACCCACAGCGUGAACCAGGGCCAGAACAUCCACCGCAAGACCACCG
CCAGCACCCGCAAAGUGAGCCUGGCCCCACAGGCCAACCUGACCGAGCU
GGACAUCUACAGCAGACGCCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCCGCAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCCGCGGCCUGC
CCCUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGUGGCAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCAGUGAUCGUGGCCUUCAUCAUGCUGCGCGCCUACUUCC
UGCAGACCAGCCAGCAGCUGAAGCAGCUGGAGAGCGAGGGCCGCAGCCC
CAUCUUCACCCACCUGGUGACCAGCCUGAAGGGCCUGUGGACCCUGCGC
GCCUUCGGCCGCCAGCCUUACUUCGAGACCCUGUUCCACAAGGCUCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGCGAGGGCCGCGUGGGCAUCA
UCCUGACCCUGGCCAUGAACAUCAUGAGCACCCUGCAGUGGGCCGUGAA
CAGCAGCAUCGACGUGGACAGCCUGAUGCGCAGCGUGAGCCGCGUGUUC
AAGUUCAUCGACAUGCCCACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCAGCAUCAGCCCCGGCCAGCGCGUGGGCCUGCUGGGCCGCAC
CGGCAGCGGCAAGAGCACCCUGCUGAGCGCCUUCCUGCGCCUGCUGAAC
ACCGAGGGCGAGAUCCAGAUCGACGGCGUGUCUUGGGACAGCAUCACCC
UGCAGCAGUGGCGCAAGGCCUUCGGCGUGAUCCCCCAGAAGGUGUUCAU
CUUCAGCGGCACCUUCCGCAAGAACCUGGACCCCUACGAGCAGUGGAGC
GACCAGGAGAUCUGGAAGGUGGCCGACGAGGUGGGCCUGCGCAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCCGCAGCGUG
CUGAGCAAGGCCAAGAUCCUGCUGCUGGACGAGCCCAGCGCCCACCUGG
ACCCCGUGACCUACCAGAUCAUCCGCCGCACCCUGAAGCAGGCCUUCGC
CGACUGCACCGUGAUCCUGUGCGAGCACCGCAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGCCAGGCCAUCAG
CCCCAGCGACCGCGUGAAGCUUUUCCCCCACCGCAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2093)
SEQ ID NO: 66
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CCCUGGAGAAGGCUAGCGUGGUGAGCAAGCUGUUCUUCAGCUGGACCAG
ACCAAUCCUGAGGAAGGGCUACAGACAGCGCCUGGAGCUGUCAGACAUC
UACCAGAUCCCUAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGUAUCGGACAGCUGGUGAGCCUGCUG
AGCAACAACCUGAACAAGUUCGACGAGGGACUGGCCCUGGCCCACUUCG
UGUGGAUCGCCCCUCUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCU
CCUGGAUCAUGCCCGGCACCAUCAAGGAGAACAUCAUCUUCGGUGUGAG
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCCGACCUGUACCUGCUGGACUCUCCCUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCAAUCAACAG
CAUCAGGAAGUUCUCCAUCGUGCAGAAGACCCCACUGCAGAUGAACGGC
AUCGAGGAGGACAGCGACGAGCCCCUGGAGAGAAGGCUGAGCCUGGUGC
CAGACUCUGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGUGGGAUCCUGAACAGAUUCUCCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACUCAAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2094)
SEQ ID NO: 67
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUGAGCAAGCUGUUCUUCAGCUGGACCAG
ACCAAUCCUGAGGAAGGGCUACAGACAGCGCCUGGAGCUGUCAGACAUC
UACCAGAUCCCCAGCGUGGACUCUGCCGACAACCUGUCUGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGUGUGUC
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCUGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGUCCUGGACCGAGACCAAGAAGCAGUCUUUCAAGCAGACCG
GAGAGUUCGGCGAGAAGAGGAAGAACAGCAUCCUGAACCCAAUCAACUC
UAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CUCCAGCAUCGACGUGGACAGCCUGAUGAGGAGCGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2095)
SEQ ID NO: 68
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CCCUGGAGAAGGCUAGCGUGGUGUCCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGCAUCGGACAGCUGGUGAGUCUGCUG
AGCAACAACCUGAACAAGUUCGACGAGGGACUGGCCCUGGCCCACUUCG
UGUGGAUCGCUCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAUCCACCGGAGC
CGGCAAGACCAGCCUGCUGAUGGUGAUCAUGGGAGAGCUGGAGCCCUCA
GAGGGCAAGAUCAAGCACAGUGGAAGAAUCUCAUUCUGCAGCCAGUUCU
CCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGUCUUUCAAGCAGACCG
GAGAGUUCGGCGAGAAGAGGAAGAACAGCAUCCUGAACCCAAUCAACAG
CAUCAGGAAGUUCAGCAUCGUGCAGAAGACCCCACUGCAGAUGAACGGC
AUCGAGGAGGACAGCGACGAGCCUCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCUCAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCUAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2096)
SEQ ID NO: 69
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CCCUGGAGAAGGCUAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCUAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGUAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCCGGCAAGAUCAGCGAGAGACUGGUGAUCACCUCAGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCUCA
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGUGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCUCUCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGUCCUGGACCGAGACCAAGAAGCAGAGCUUCAAGCAGACCG
GAGAGUUCGGCGAGAAGAGGAAGAACAGCAUCCUGAACCCAAUCAACUC
UAUCAGGAAGUUCAGCAUCGUGCAGAAGACCCCACUGCAGAUGAACGGC
AUCGAGGAGGACAGCGACGAGCCCCUGGAGAGAAGGCUGUCCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCUAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CAGCAGCAUCGACGUGGACAGCCUGAUGAGGAGCGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCUCAAUCAGCCCUGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2097)
SEQ ID NO: 70
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCCAGCGUGGUGAGCAAGCUGUUCUUCAGCUGGACCAG
ACCAAUCCUGCGCAAGGGCUACCGCCAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGCAGGUGCUUCUUCUGGAGAUUCAUGUUCUACGGAAUC
UUCCUGUACCUGGGCGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCAGCCAUCUUCGGCCUGCACCACAUCGGAAUGCAGA
UGCGCAUCGCCAUGUUCAGCCUGAUCUACAAGAAGACCCUGAAGCUGAG
CAGCCGCGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGCCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGCGCGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGCGCUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGCAAGAUCUUCACCACCAUCAGCUUCUGCAUCGUGCUGCGCA
UGGCCGUGACCCGCCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGACAGCUGCUGGCCGUGGCCGGAAGCACCGGAGC
CGGCAAGACCAGCCUGCUGAUGGUGAUCAUGGGCGAGCUGGAGCCCAGC
GAGGGCAAGAUCAAGCACAGCGGACGCAUCAGCUUCUGCAGCCAGUUCA
GCUGGAUCAUGCCCGGCACCAUCAAGGAGAACAUCAUCUUCGGCGUGAG
CUACGACGAGUACAGAUACCGCAGCGUGAUCAAGGCCUGCCAGCUGGAG
GAGGACAUCAGCAAGUUCGCAGAGAAGGACAACAUCGUGCUGGGCGAGG
GCGGCAUCACCCUGAGCGGCGGCCAGAGGGCCAGAAUCAGCCUGGCACG
CGCAGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGAGAAG
AAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GCGAGUUCGGCGAGAAGCGCAAGAACAGCAUCCUGAACCCCAUCAACAG
CAUCAGGAAGUUCAGCAUCGUGCAGAAGACCCCACUGCAGAUGAACGGC
AUCGAGGAGGACAGCGACGAGCCCCUGGAGCGCAGGCUGAGCCUGGUGC
CAGACAGCGAGCAGGGCGAGGCCAUCCUGCCCCGCAUCAGCGUGAUCAG
CACCGGCCCCACCCUGCAGGCCAGGCGCCGCCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGACGCCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCCGCAGCCC
CAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGCGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGCGCGUGGGCCUGCUGGGACGCAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCCCAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCCGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACCGCGUGAAGCUUUUCCCCCACCGCAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2098)
SEQ ID NO: 71
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGCGCAGCC
CCCUGGAGAAGGCCAGCGUGGUGAGCAAGCUGUUCUUCAGCUGGACCCG
CCCCAUCCUGCGCAAGGGCUACCGCCAGCGCCUGGAGCUGAGCGACAUC
UACCAGAUCCCCAGCGUGGACAGCGCCGACAACCUGAGCGAGAAGCUGG
AGCGCGAGUGGGACAGAGAGCUGGCCAGCAAGAAGAACCCCAAGCUGAU
CAACGCCCUGCGCAGGUGCUUCUUCUGGCGCUUCAUGUUCUACGGAAUC
UUCCUGUACCUGGGCGAGGUGACCAAGGCCGUGCAGCCCCUGCUGCUGG
GCCGCAUCAUCGCCAGCUACGACCCCGACAACAAGGAGGAGCGCAGCAU
CGCCAUCUACCUGGGCAUCGGCCUGUGCCUGCUGUUCAUCGUGCGCACC
CUGCUGCUGCACCCCGCCAUCUUCGGCCUGCACCACAUCGGAAUGCAGA
UGCGCAUCGCCAUGUUCAGCCUGAUCUACAAGAAGACCCUGAAGCUGAG
CAGCCGCGUGCUGGACAAGAUCAGCAUCGGCCAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGCCUGAUCUGGGA
GCUGCUGCAGGCCAGCGCCUUCUGCGGCCUGGGCUUCCUGAUCGUGCUG
GCCCUGUUCCAGGCCGGCCUGGGCCGCAUGAUGAUGAAGUACCGCGACC
AGCGCGCCGGCAAGAUCAGCGAGAGACUGGUGAUCACCAGCGAGAUGAU
CGAGAACAUCCAGAGCGUGAAGGCCUACUGCUGGGAGGAGGCCAUGGAG
AAGAUGAUCGAGAACCUGAGACAGACCGAGCUGAAGCUGACCCGGAAGG
CCGCCUACGUGCGCUACUUCAACAGCAGCGCCUUCUUCUUCAGCGGCUU
CUUCGUGGUGUUCCUGAGCGUGCUGCCCUACGCCCUGAUCAAGGGCAUC
AUCCUGCGCAAGAUCUUCACCACCAUCAGCUUCUGCAUCGUGCUGCGCA
UGGCCGUGACCCGCCAGUUCCCCUGGGCCGUGCAGACCUGGUACGACAG
CCUGGGCGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGAAGCACCGGAGC
CGGCAAGACCAGCCUGCUGAUGGUGAUCAUGGGCGAGCUGGAGCCCAGC
GAGGGCAAGAUCAAGCACAGCGGCCGCAUCAGCUUCUGCAGCCAGUUCA
GCUGGAUCAUGCCCGGCACCAUCAAGGAGAACAUCAUCUUCGGCGUGAG
CUACGACGAGUACCGCUACCGCAGCGUGAUCAAGGCCUGCCAGCUGGAG
GAGGACAUCAGCAAGUUCGCCGAGAAGGACAACAUCGUGCUGGGCGAGG
GCGGCAUCACCCUGAGCGGCGGCCAGCGCGCCCGCAUCAGCCUGGCCCG
CGCCGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGAGAAG
AAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GCGAGUUCGGCGAGAAGCGCAAGAACAGCAUCCUGAACCCCAUCAACAG
CAUCCGCAAGUUCAGCAUCGUGCAGAAGACCCCACUGCAGAUGAACGGC
AUCGAGGAGGACAGCGACGAGCCCCUGGAGCGCCGCCUGAGCCUGGUGC
CCGACAGCGAGCAGGGCGAGGCCAUCCUGCCCCGCAUCAGCGUGAUCAG
CACCGGCCCCACCCUGCAGGCCCGCCGCCGCCAGAGCGUGCUGAACCUG
AUGACCCACAGCGUGAACCAGGGCCAGAACAUCCACCGCAAGACCACCG
CCAGCACCCGCAAAGUGAGCCUGGCCCCACAGGCCAACCUGACCGAGCU
GGACAUCUACAGCAGACGCCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGCGCUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGCGUGGCCGACACCCUGCUGGCCAUGGGCUUCUUCCGCGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACCGCUUCAGCAAGGACAUCGCCAUCCUGGACG
ACCUGCUGCCCCUGACCAUCUUCGACUUCAUCCAGCUGCUGCUGAUCGU
GAUCGGCGCCAUCGCCGUGGUGGCCGUGCUGCAGCCCUACAUCUUCGUG
GCCACCGUGCCCGUGAUCGUGGCCUUCAUCAUGCUGAGAGCCUACUUCC
UGCAGACCAGCCAGCAGCUGAAGCAGCUGGAGAGCGAGGGCCGCAGCCC
CAUCUUCACCCACCUGGUGACCAGCCUGAAGGGCCUGUGGACCCUGAGG
GCCUUCGGCCGCCAGCCCUACUUCGAGACCCUGUUCCACAAGGCCCUGA
ACCUGCACACCGCCAACUGGUUCCUGUACCUGAGCACCCUGCGCUGGUU
CCAGAUGCGCAUCGAGAUGAUCUUCGUGAUCUUCUUCAUCGCCGUGACC
UUCAUCAGCAUCCUGACCACCGGCGAGGGCGAGGGACGCGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCCACCGAGGGCAAGCCCACCAAGAGCACCAAGC
CCUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGCGCGUGGGCCUGCUGGGCCGCAC
CGGCAGCGGCAAGAGCACCCUGCUGAGCGCCUUCCUGCGCCUGCUGAAC
ACCGAGGGCGAGAUCCAGAUCGACGGCGUGAGCUGGGACAGCAUCACCC
UGCAGCAGUGGCGCAAGGCCUUCGGCGUGAUCCCCCAGAAGGUGUUCAU
CUUCAGCGGCACCUUCCGCAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCCGCAGCGUG
CUGAGCAAGGCCAAGAUCCUGCUGCUGGACGAGCCCAGCGCCCACCUGG
ACCCCGUGACCUACCAGAUCAUCAGACGCACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGCCAGUACGACA
GCAUCCAGAAGCUGCUGAACGAGCGCAGCCUGUUCCGCCAGGCCAUCAG
CCCCAGCGACCGCGUGAAGCUUUUCCCCCACCGCAACAGCAGCAAGUGC
AAGAGCAAGCCCCAGAUCGCCGCCCUGAAGGAGGAGACCGAGGAGGAGG
UGCAGGACACCCGCCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2099)
SEQ ID NO: 72
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCAAUCAACAG
CAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2101)
SEQ ID NO: 73
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCAGCUUCUGCAGCCAGUUCU
CCUGGAUCAUGCCCGGCACCAUCAAGGAGAACAUCAUCUUCGGUGUGUC
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCCGACCUGUACCUGCUGGACUCUCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCACUGCAGAUGAACGGC
AUCGAGGAGGACUCUGACGAGCCCCUGGAGAGAAGGCUGUCCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2102)
SEQ ID NO: 74
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCAGCCUGCUGAUGGUGAUCAUGGGAGAGCUGGAGCCUAGC
GAGGGCAAGAUCAAGCACAGUGGAAGAAUCUCAUUCUGCUCUCAGUUCA
GCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GUGGCAUCACCCUGAGCGGAGGCCAGAGGGCCAGAAUCAGCCUGGCAAG
AGCAGUGUACAAGGACGCCGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCUGUGAGCUGGACCGAGACCAAGAAGCAGUCUUUCAAGCAGACCG
GAGAGUUCGGCGAGAAGAGGAAGAACUCUAUCCUGAACCCAAUCAACAG
CAUCAGGAAGUUCUCCAUCGUGCAGAAGACCCCACUGCAGAUGAACGGC
AUCGAGGAGGACAGCGACGAGCCUCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2103)
SEQ ID NO: 75
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CUCUGGAGAAGGCUAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCAGCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCAAUCAACAG
CAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2104)
SEQ ID NO: 76
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCUAGCGUGGUGUCCAAGCUGUUCUUCAGCUGGACCAG
ACCAAUCCUGAGGAAGGGCUACAGACAGCGCCUGGAGCUGAGCGACAUC
UACCAGAUCCCCUCUGUGGACAGCGCUGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCAAUCAACAG
CAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCAGCAUCCUGACCACCGGCGAGGGAGAGGGAAGAGUGGGCAUCA
UCCUGACCCUGGCCAUGAACAUCAUGAGCACCCUGCAGUGGGCCGUGAA
CAGCAGCAUCGACGUGGACAGCCUGAUGAGGAGCGUGAGCAGGGUGUUC
AAGUUCAUCGACAUGCCAACCGAGGGCAAGCCCACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCAGCUUCAGCAUCAGCCCCGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGAGCUGGGACAGCAUCACCC
UGCAGCAGUGGAGGAAGGCCUUCGGCGUGAUCCCACAGAAGGUGUUCAU
CUUCAGCGGAACCUUCAGAAAGAACCUGGACCCCUACGAGCAGUGGAGC
GACCAGGAGAUCUGGAAGGUGGCCGACGAGGUGGGCCUGAGAAGCGUGA
UCGAGCAGUUCCCCGGCAAGCUGGACUUCGUGCUGGUGGACGGGGGCUG
CGUGCUGAGCCACGGCCACAAGCAGCUGAUGUGCCUGGCCAGAAGCGUG
CUGAGCAAGGCCAAGAUCCUGCUGCUGGACGAGCCCAGCGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACA
GCAUCCAGAAGCUGCUGAACGAGAGGAGCCUGUUCCGGCAGGCCAUCAG
CCCCAGCGACAGGGUGAAGCUGUUCCCCCACCGGAACAGCAGCAAGUGC
AAGAGCAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2105)
SEQ ID NO: 77
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGCGCAGCC
CCCUGGAGAAGGCCAGCGUGGUGAGCAAGCUGUUCUUCAGCUGGACCCG
CCCCAUCCUGCGCAAGGGCUACCGCCAGCGCCUGGAGCUGAGCGACAUC
UACCAGAUCCCCAGCGUGGACAGCGCCGACAACCUGAGCGAGAAGCUGG
AGCGCGAGUGGGACCGCGAGCUGGCCAGCAAGAAGAACCCCAAGCUGAU
CAACGCCCUGCGCCGCUGCUUCUUCUGGAGAUUCAUGUUCUACGGAAUC
UUCCUAUACCUAGGGGAAGUCACCAAAGCAGUACAGCCACUCCUACUGG
GAAGAAUCAUAGCAAGCUACGACCCGGACAACAAGGAGGAACGCAGUAU
CGCGAUAUACCUAGGCAUAGGCCUAUGCCUACUCUUCAUAGUGAGGACA
CUGCUCCUACACCCAGCCAUAUUCGGCCUACAUCACAUAGGAAUGCAGA
UGAGAAUAGCAAUGUUCAGUCUAAUAUACAAGAAGACACUAAAGCUGUC
AAGCCGAGUACUAGACAAAAUAAGUAUAGGACAACUAGUAAGUCUCCUA
AGCAACAACCUGAACAAAUUCGACGAAGGACUAGCACUAGCACAUUUCG
UGUGGAUCGCACCACUACAAGUGGCACUCCUCAUGGGGCUAAUCUGGGA
GCUACUACAGGCGAGUGCCUUCUGCGGACUAGGUUUCCUGAUAGUCCUA
GCCCUAUUCCAGGCAGGGCUAGGGAGAAUGAUGAUGAAGUACAGAGACC
AGAGAGCAGGGAAGAUCAGUGAAAGACUAGUGAUAACCUCAGAAAUGAU
AGAAAACAUCCAAAGUGUAAAGGCAUACUGCUGGGAAGAAGCAAUGGAG
AAAAUGAUAGAAAACCUAAGACAAACAGAACUGAAACUGACACGGAAGG
CAGCCUACGUGAGAUACUUCAACAGCUCAGCCUUCUUCUUCUCAGGGUU
CUUCGUGGUGUUCCUGAGCGUGCUGCCCUACGCCCUGAUCAAGGGCAUC
AUCCUGCGCAAGAUCUUCACCACCAUCUCAUUCUGCAUAGUACUGCGCA
UGGCGGUCACACGGCAAUUCCCCUGGGCAGUACAAACAUGGUACGACAG
UCUAGGAGCAAUAAACAAAAUACAGGACUUCCUACAAAAGCAAGAAUAC
AAGACACUAGAAUACAACCUAACGACCACCGAGGUGGUGAUGGAGAACG
UGACCGCCUUCUGGGAGGAGGGCUUCGGCGAGCUGUUCGAGAAGGCCAA
GCAGAACAACAACAACCGCAAGACCAGCAACGGCGACGACAGCCUGUUC
UUCAGCAACUUCAGCCUGCUGGGCACCCCCGUGCUGAAGGACAUCAACU
UCAAGAUCGAGCGCGGCCAGCUGCUGGCCGUGGCCGGCAGCACCGGCGC
CGGCAAGACCAGCCUGCUGAUGGUGAUCAUGGGCGAGCUGGAGCCCAGC
GAGGGCAAGAUCAAGCACAGCGGCCGCAUCAGCUUCUGCAGCCAGUUCA
GCUGGAUCAUGCCCGGCACCAUCAAGGAGAACAUCAUCUUCGGCGUGAG
CUACGACGAGUACCGCUACCGCAGCGUGAUCAAGGCCUGCCAGCUGGAG
GAGGACAUCAGCAAGUUCGCCGAGAAGGACAACAUCGUGCUGGGCGAGG
GCGGCAUCACCCUGAGCGGCGGCCAGCGCGCCCGCAUCAGCCUGGCCCG
CGCCGUGUACAAGGACGCCGACCUGUACCUGCUGGACAGCCCCUUCGGC
UACCUGGACGUGCUGACCGAGAAGGAGAUCUUCGAGAGCUGCGUGUGCA
AGCUGAUGGCCAACAAGACCCGCAUCCUGGUGACCAGCAAGAUGGAGCA
CCUGAAGAAGGCCGACAAGAUCCUGAUCCUGCACGAGGGCAGCAGCUAC
UUCUACGGCACCUUCAGCGAGCUGCAGAACCUGCAGCCCGACUUCAGCA
GCAAGCUGAUGGGCUGCGACAGCUUCGACCAGUUCAGCGCCGAGCGCCG
CAACAGCAUCCUGACCGAGACCCUGCACCGCUUCAGCCUGGAGGGCGAC
GCCCCCGUGAGCUGGACCGAGACCAAGAAGCAGAGCUUCAAGCAGACCG
GCGAGUUCGGCGAGAAGCGCAAGAACAGCAUCCUGAACCCCAUCAACAG
CAUCCGCAAGUUCAGCAUCGUGCAGAAGACCCCACUGCAGAUGAACGGC
AUCGAGGAGGACAGCGACGAGCCCCUGGAGCGCCGCCUGAGCCUGGUGC
CCGACAGCGAGCAGGGCGAGGCCAUCCUGCCCCGCAUCAGCGUGAUCAG
CACCGGCCCCACCCUGCAGGCCCGCCGCCGCCAGAGCGUGCUGAACCUG
AUGACCCACAGCGUGAACCAGGGCCAGAACAUCCACCGCAAGACCACCG
CCAGCACCCGCAAGGUGAGCCUGGCCCCACAGGCCAACCUGACCGAGCU
GGACAUCUACAGCCGCCGCCUGAGCCAGGAGACCGGCCUGGAGAUCAGC
GAGGAGAUCAACGAGGAGGACCUGAAGGAGUGCUUCUUCGACGACAUGG
AGAGCAUCCCCGCCGUGACCACCUGGAACACCUACCUGCGCUACAUCAC
CGUGCACAAGAGCCUAAUAUUCGUGCUAAUAUGGUGCCUAGUAAUAUUC
CUGGCAGAGGUGGCAGCAAGUCUAGUAGUGCUGUGGCUCCUAGGAAACA
CACCACUACAAGACAAAGGGAACAGUACACAUAGUAGAAACAACAGCUA
CGCAGUGAUAAUCACCAGCACCAGUUCGUACUACGUGUUCUACAUAUAC
GUGGGAGUAGCCGACACACUACUAGCAAUGGGAUUCUUCAGAGGUCUAC
CACUGGUGCAUACACUAAUCACAGUGUCGAAAAUACUACACCACAAAAU
GCUACAUAGUGUACUACAAGCACCAAUGUCAACCCUCAACACGCUAAAA
GCAGGUGGGAUACUAAACAGAUUCAGCAAAGACAUAGCAAUACUAGACG
ACCUACUGCCACUAACCAUAUUCGACUUCAUCCAGCUACUACUAAUAGU
GAUAGGAGCAAUAGCAGUAGUCGCAGUACUACAACCCUACAUCUUCGUA
GCAACAGUGCCAGUGAUAGUGGCAUUCAUAAUGCUAAGAGCAUACUUCC
UCCAAACCUCACAGCAACUCAAACAACUGGAAAGUGAAGGCAGGAGUCC
AAUAUUCACACAUCUAGUAACAAGCCUAAAAGGACUAUGGACACUACGA
GCCUUCGGACGGCAGCCAUACUUCGAAACACUGUUCCACAAAGCACUGA
ACCUACAUACAGCCAACUGGUUCCUAUACCUGUCAACACUGCGCUGGUU
CCAAAUGAGAAUAGAAAUGAUAUUCGUCAUCUUCUUCAUAGCAGUAACC
UUCAUCAGCAUCCUGACCACCGGCGAGGGCGAGGGCCGCGUGGGCAUAA
UCCUGACACUAGCCAUGAACAUCAUGAGUACACUACAGUGGGCAGUAAA
CAGCAGCAUAGACGUGGACAGCCUAAUGCGAAGUGUGAGCCGAGUCUUC
AAGUUCAUAGACAUGCCAACAGAAGGUAAACCAACCAAGUCAACCAAAC
CAUACAAGAACGGCCAACUCUCGAAAGUAAUGAUAAUAGAGAACUCACA
CGUGAAGAAAGACGACAUCUGGCCCUCAGGGGGCCAGAUGACCGUGAAG
GACCUGACCGCCAAGUACACCGAGGGCGGCAACGCCAUCCUGGAGAACA
UCAGCUUCAGCAUCAGCCCCGGCCAGCGCGUGGGCCUGCUGGGCCGCAC
CGGCAGCGGCAAGAGCACCCUGCUGAGCGCCUUCCUGCGCCUGCUGAAC
ACCGAGGGCGAGAUCCAGAUCGACGGCGUGAGCUGGGACAGCAUCACCC
UGCAGCAGUGGCGCAAGGCCUUCGGCGUGAUCCCCCAGAAGGUGUUCAU
CUUCAGCGGCACCUUCCGCAAGAACCUGGACCCCUACGAGCAGUGGAGC
GACCAGGAGAUCUGGAAGGUGGCCGACGAGGUGGGCCUGCGCAGCGUGA
UCGAGCAGUUCCCCGGCAAGCUGGACUUCGUGCUGGUGGACGGCGGCUG
CGUGCUGAGCCACGGCCACAAGCAGCUGAUGUGCCUGGCCCGCAGCGUG
CUGAGCAAGGCCAAGAUCCUGCUGCUGGACGAGCCCAGCGCCCACCUGG
ACCCCGUGACCUACCAGAUCAUCCGCCGCACCCUGAAGCAGGCCUUCGC
CGACUGCACCGUGAUCCUGUGCGAGCACCGCAUCGAGGCCAUGCUGGAG
UGCCAGCAGUUCCUGGUGAUCGAGGAGAACAAGGUGCGCCAGUACGACA
GCAUCCAGAAGCUGCUGAACGAGCGCAGCCUGUUCCGCCAGGCCAUCAG
CCCCAGCGACCGCGUGAAGCUGUUCCCCCACCGCAACAGCAGCAAGUGC
AAGAGCAAGCCCCAGAUCGCCGCCCUGAAGGAGGAGACCGAGGAGGAGG
UGCAGGACACCCGCCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2106)
SEQ ID NO: 78
ACGCGAAAAAAUGCGUACAACAAACUUGCGUAAACCAAAAAAAUGCCAC
CAUGCAGAGGAGCCCCCUGGAGAAGGCUAGCGUGGUGAGCAAGCUGUUC
UUCAGCUGGACCAGACCAAUCCUGAGGAAGGGCUACAGACAGCGCCUGG
AGCUGAGCGACAUCUACCAGAUCCCCAGCGUGGACAGCGCCGACAACCU
GAGCGAGAAGCUGGAGAGAGAGUGGGACAGAGAGCUGGCCAGCAAGAAG
AACCCCAAGCUGAUCAACGCCCUGCGGAGGUGCUUCUUCUGGAGAUUCA
UGUUCUACGGAAUCUUCCUGUACCUGGGGGAGGUGACCAAGGCCGUGCA
GCCCCUGCUGCUGGGAAGAAUCAUCGCCAGCUACGACCCCGACAACAAG
GAGGAGCGCAGCAUCGCCAUCUACCUGGGCAUCGGCCUGUGCCUGCUGU
UCAUCGUGAGGACCCUGCUGCUGCACCCAGCCAUCUUCGGCCUGCACCA
CAUCGGAAUGCAGAUGAGAAUCGCCAUGUUCAGCCUGAUCUACAAGAAG
ACCCUGAAGCUGAGCAGCAGGGUGCUGGACAAGAUCAGCAUCGGACAGC
UGGUGAGCCUGCUGAGCAACAACCUGAACAAGUUCGACGAGGGACUGGC
CCUGGCCCACUUCGUGUGGAUCGCCCCACUGCAGGUGGCCCUGCUGAUG
GGGCUGAUCUGGGAGCUGCUGCAGGCCAGCGCCUUCUGCGGCCUGGGCU
UCCUGAUCGUGCUGGCCCUGUUCCAGGCCGGCCUGGGCAGAAUGAUGAU
GAAGUACAGAGACCAGAGAGCCGGCAAGAUCAGCGAGAGACUGGUGAUC
ACCAGCGAGAUGAUCGAGAACAUCCAGAGCGUGAAGGCAUACUGCUGGG
AGGAGGCCAUGGAGAAGAUGAUCGAGAACCUGAGACAGACCGAGCUGAA
GCUGACCCGGAAGGCCGCCUACGUGAGAUACUUCAACAGCAGCGCCUUC
UUCUUCAGCGGGUUCUUCGUGGUGUUCCUGAGCGUGCUGCCCUACGCCC
UGAUCAAGGGCAUCAUCCUGCGGAAGAUCUUCACCACCAUCAGCUUCUG
CAUCGUGCUGCGCAUGGCCGUGACCCGGCAGUUCCCCUGGGCCGUGCAG
ACCUGGUACGACAGCCUGGGAGCCAUCAACAAGAUCCAGGACUUCCUGC
AGAAGCAGGAGUACAAGACCCUGGAGUACAACCUGACCACCACCGAGGU
GGUGAUGGAGAACGUGACCGCCUUCUGGGAGGAGGGAUUCGGCGAGCUG
UUCGAGAAGGCCAAGCAGAACAACAACAACAGAAAGACCAGCAACGGCG
ACGACAGCCUGUUCUUCAGCAACUUCAGCCUGCUGGGCACCCCCGUGCU
GAAGGACAUCAACUUCAAGAUCGAGAGAGGACAGCUGCUGGCCGUGGCC
GGAAGCACCGGAGCCGGCAAGACCAGCCUGCUGAUGGUGAUCAUGGGAG
AGCUGGAGCCCAGCGAGGGCAAGAUCAAGCACAGCGGAAGAAUCAGCUU
CUGCAGCCAGUUCAGCUGGAUCAUGCCCGGCACCAUCAAGGAGAACAUC
AUCUUCGGCGUGAGCUACGACGAGUACAGAUACAGAAGCGUGAUCAAGG
CCUGCCAGCUGGAGGAGGACAUCAGCAAGUUCGCAGAGAAGGACAACAU
CGUGCUGGGAGAGGGCGGCAUCACCCUGAGCGGAGGCCAGAGGGCCAGA
AUCAGCCUGGCAAGAGCAGUGUACAAGGACGCCGACCUGUACCUGCUGG
ACAGCCCCUUCGGAUACCUGGACGUGCUGACCGAGAAGGAGAUCUUCGA
GAGCUGCGUGUGCAAGCUGAUGGCCAACAAGACCAGGAUCCUGGUGACC
AGCAAGAUGGAGCACCUGAAGAAGGCCGACAAGAUCCUGAUCCUGCACG
AGGGCAGCAGCUACUUCUACGGGACCUUCAGCGAGCUGCAGAACCUGCA
GCCAGACUUCAGCAGCAAGCUGAUGGGCUGCGACAGCUUCGACCAGUUC
AGCGCCGAGAGAAGAAACAGCAUCCUGACCGAGACCCUGCACAGGUUCA
GCCUGGAGGGCGACGCCCCCGUGAGCUGGACCGAGACCAAGAAGCAGAG
CUUCAAGCAGACCGGAGAGUUCGGCGAGAAGAGGAAGAACAGCAUCCUG
AACCCAAUCAACAGCAUCAGGAAGUUCAGCAUCGUGCAGAAGACCCCAC
UGCAGAUGAACGGCAUCGAGGAGGACAGCGACGAGCCCCUGGAGAGAAG
GCUGAGCCUGGUGCCAGACAGCGAGCAGGGCGAGGCCAUCCUGCCCCGC
AUCAGCGUGAUCAGCACCGGCCCCACCCUGCAGGCCAGGAGGAGGCAGA
GCGUGCUGAACCUGAUGACCCACAGCGUGAACCAGGGCCAGAACAUCCA
CAGGAAGACCACCGCCAGCACCAGGAAGGUGAGCCUGGCCCCACAGGCC
AACCUGACCGAGCUGGACAUCUACAGCAGAAGGCUGAGCCAGGAGACCG
GCCUGGAGAUCAGCGAGGAGAUCAACGAGGAGGACCUGAAGGAGUGCUU
CUUCGACGACAUGGAGAGCAUCCCAGCCGUGACCACCUGGAACACCUAC
CUGAGGUACAUCACCGUGCACAAGAGCCUGAUCUUCGUGCUGAUCUGGU
GCCUGGUGAUCUUCCUGGCCGAGGUGGCCGCCAGCCUGGUGGUGCUGUG
GCUGCUGGGCAACACCCCACUGCAGGACAAGGGCAACAGCACCCACAGC
AGAAACAACAGCUACGCCGUGAUCAUCACCAGCACCAGCAGCUACUACG
UGUUCUACAUCUACGUGGGAGUGGCCGACACCCUGCUGGCCAUGGGCUU
CUUCAGAGGCCUGCCACUGGUGCACACCCUGAUCACCGUGAGCAAGAUC
CUGCACCACAAGAUGCUGCACAGCGUGCUGCAGGCCCCCAUGAGCACCC
UGAACACCCUGAAGGCCGGCGGGAUCCUGAACAGAUUCAGCAAGGACAU
CGCCAUCCUGGACGACCUGCUGCCCCUGACCAUCUUCGACUUCAUCCAG
CUGCUGCUGAUCGUGAUCGGAGCCAUCGCCGUGGUGGCCGUGCUGCAGC
CCUACAUCUUCGUGGCCACCGUGCCAGUGAUCGUGGCCUUCAUCAUGCU
GAGAGCCUACUUCCUGCAGACCAGCCAGCAGCUGAAGCAGCUGGAGAGC
GAGGGCAGGAGCCCAAUCUUCACCCACCUGGUGACCAGCCUGAAGGGAC
UGUGGACCCUGAGGGCCUUCGGCCGGCAGCCCUACUUCGAGACCCUGUU
CCACAAGGCCCUGAACCUGCACACCGCCAACUGGUUCCUGUACCUGAGC
ACCCUGCGCUGGUUCCAGAUGAGAAUCGAGAUGAUCUUCGUGAUCUUCU
UCAUCGCCGUGACCUUCAUCAGCAUCCUGACCACCGGCGAGGGAGAGGG
AAGAGUGGGCAUCAUCCUGACCCUGGCCAUGAACAUCAUGAGCACCCUG
CAGUGGGCCGUGAACAGCAGCAUCGACGUGGACAGCCUGAUGAGGAGCG
UGAGCAGGGUGUUCAAGUUCAUCGACAUGCCAACCGAGGGCAAGCCCAC
CAAGAGCACCAAGCCAUACAAGAACGGCCAGCUGAGCAAGGUGAUGAUC
AUCGAGAACAGCCACGUGAAGAAGGACGACAUCUGGCCCAGCGGCGGCC
AGAUGACCGUGAAGGACCUGACCGCCAAGUACACCGAGGGCGGCAACGC
CAUCCUGGAGAACAUCAGCUUCAGCAUCAGCCCCGGCCAGAGGGUGGGC
CUGCUGGGAAGAACCGGCAGCGGCAAGAGCACCCUGCUGAGCGCCUUCC
UGAGACUGCUGAACACCGAGGGCGAGAUCCAGAUCGACGGCGUGAGCUG
GGACAGCAUCACCCUGCAGCAGUGGAGGAAGGCCUUCGGCGUGAUCCCA
CAGAAGGUGUUCAUCUUCAGCGGAACCUUCAGAAAGAACCUGGACCCCU
ACGAGCAGUGGAGCGACCAGGAGAUCUGGAAGGUGGCCGACGAGGUGGG
CCUGAGAAGCGUGAUCGAGCAGUUCCCCGGCAAGCUGGACUUCGUGCUG
GUGGACGGGGGCUGCGUGCUGAGCCACGGCCACAAGCAGCUGAUGUGCC
UGGCCAGAAGCGUGCUGAGCAAGGCCAAGAUCCUGCUGCUGGACGAGCC
CAGCGCCCACCUGGACCCAGUGACCUACCAGAUCAUCAGAAGAACCCUG
AAGCAGGCCUUCGCCGACUGCACCGUGAUCCUGUGCGAGCACAGGAUCG
AGGCCAUGCUGGAGUGCCAGCAGUUCCUGGUGAUCGAGGAGAACAAGGU
GCGGCAGUACGACAGCAUCCAGAAGCUGCUGAACGAGAGGAGCCUGUUC
CGGCAGGCCAUCAGCCCCAGCGACAGGGUGAAGCUGUUCCCCCACCGGA
ACAGCAGCAAGUGCAAGAGCAAGCCCCAGAUCGCCGCCCUGAAGGAGGA
GACCGAGGAGGAGGUGCAGGACACCAGGCUGUAGAUAAGUGAACUCGAG
CUAGUGACUGACUAGGAUCUGGUUACCACUAAACCAGCCUCAAGAACAC
CCGAAUGGAGUCUCUAAGCUACAUAAUACCAACUUACACUUACAAAAUG
UUGUCCCCCAAAAUGUAGCCAUUCGUAUCUGCUCCUAAUAAAAAGAAAG
UUUCUUCACAUUCUAG
(mARM2107)
SEQ ID NO: 79
AGCGCAGGGCGGUAACUCUGGGCGGGGCUGGGCUCCAGGGCUGGACAGC
ACAGUCCCUCUGAACUGCACAGAGACCUCGCAGGCCCCGAGAACUGUCG
CCCUUCCACGCCACCAUGCAGAGGAGCCCCCUGGAGAAGGCUAGCGUGG
UGAGCAAGCUGUUCUUCAGCUGGACCAGACCAAUCCUGAGGAAGGGCUA
CAGACAGCGCCUGGAGCUGAGCGACAUCUACCAGAUCCCCAGCGUGGAC
AGCGCCGACAACCUGAGCGAGAAGCUGGAGAGAGAGUGGGACAGAGAGC
UGGCCAGCAAGAAGAACCCCAAGCUGAUCAACGCCCUGCGGAGGUGCUU
CUUCUGGAGAUUCAUGUUCUACGGAAUCUUCCUGUACCUGGGGGAGGUG
ACCAAGGCCGUGCAGCCCCUGCUGCUGGGAAGAAUCAUCGCCAGCUACG
ACCCCGACAACAAGGAGGAGCGCAGCAUCGCCAUCUACCUGGGCAUCGG
CCUGUGCCUGCUGUUCAUCGUGAGGACCCUGCUGCUGCACCCAGCCAUC
UUCGGCCUGCACCACAUCGGAAUGCAGAUGAGAAUCGCCAUGUUCAGCC
UGAUCUACAAGAAGACCCUGAAGCUGAGCAGCAGGGUGCUGGACAAGAU
CAGCAUCGGACAGCUGGUGAGCCUGCUGAGCAACAACCUGAACAAGUUC
GACGAGGGACUGGCCCUGGCCCACUUCGUGUGGAUCGCCCCACUGCAGG
UGGCCCUGCUGAUGGGGCUGAUCUGGGAGCUGCUGCAGGCCAGCGCCUU
CUGCGGCCUGGGCUUCCUGAUCGUGCUGGCCCUGUUCCAGGCCGGCCUG
GGCAGAAUGAUGAUGAAGUACAGAGACCAGAGAGCCGGCAAGAUCAGCG
AGAGACUGGUGAUCACCAGCGAGAUGAUCGAGAACAUCCAGAGCGUGAA
GGCAUACUGCUGGGAGGAGGCCAUGGAGAAGAUGAUCGAGAACCUGAGA
CAGACCGAGCUGAAGCUGACCCGGAAGGCCGCCUACGUGAGAUACUUCA
ACAGCAGCGCCUUCUUCUUCAGCGGGUUCUUCGUGGUGUUCCUGAGCGU
GCUGCCCUACGCCCUGAUCAAGGGCAUCAUCCUGCGGAAGAUCUUCACC
ACCAUCAGCUUCUGCAUCGUGCUGCGCAUGGCCGUGACCCGGCAGUUCC
CCUGGGCCGUGCAGACCUGGUACGACAGCCUGGGAGCCAUCAACAAGAU
CCAGGACUUCCUGCAGAAGCAGGAGUACAAGACCCUGGAGUACAACCUG
ACCACCACCGAGGUGGUGAUGGAGAACGUGACCGCCUUCUGGGAGGAGG
GAUUCGGCGAGCUGUUCGAGAAGGCCAAGCAGAACAACAACAACAGAAA
GACCAGCAACGGCGACGACAGCCUGUUCUUCAGCAACUUCAGCCUGCUG
GGCACCCCCGUGCUGAAGGACAUCAACUUCAAGAUCGAGAGAGGACAGC
UGCUGGCCGUGGCCGGAAGCACCGGAGCCGGCAAGACCAGCCUGCUGAU
GGUGAUCAUGGGAGAGCUGGAGCCCAGCGAGGGCAAGAUCAAGCACAGC
GGAAGAAUCAGCUUCUGCAGCCAGUUCAGCUGGAUCAUGCCCGGCACCA
UCAAGGAGAACAUCAUCUUCGGCGUGAGCUACGACGAGUACAGAUACAG
AAGCGUGAUCAAGGCCUGCCAGCUGGAGGAGGACAUCAGCAAGUUCGCA
GAGAAGGACAACAUCGUGCUGGGAGAGGGCGGCAUCACCCUGAGCGGAG
GCCAGAGGGCCAGAAUCAGCCUGGCAAGAGCAGUGUACAAGGACGCCGA
CCUGUACCUGCUGGACAGCCCCUUCGGAUACCUGGACGUGCUGACCGAG
AAGGAGAUCUUCGAGAGCUGCGUGUGCAAGCUGAUGGCCAACAAGACCA
GGAUCCUGGUGACCAGCAAGAUGGAGCACCUGAAGAAGGCCGACAAGAU
CCUGAUCCUGCACGAGGGCAGCAGCUACUUCUACGGGACCUUCAGCGAG
CUGCAGAACCUGCAGCCAGACUUCAGCAGCAAGCUGAUGGGCUGCGACA
GCUUCGACCAGUUCAGCGCCGAGAGAAGAAACAGCAUCCUGACCGAGAC
CCUGCACAGGUUCAGCCUGGAGGGCGACGCCCCCGUGAGCUGGACCGAG
ACCAAGAAGCAGAGCUUCAAGCAGACCGGAGAGUUCGGCGAGAAGAGGA
AGAACAGCAUCCUGAACCCAAUCAACAGCAUCAGGAAGUUCAGCAUCGU
GCAGAAGACCCCACUGCAGAUGAACGGCAUCGAGGAGGACAGCGACGAG
CCCCUGGAGAGAAGGCUGAGCCUGGUGCCAGACAGCGAGCAGGGCGAGG
CCAUCCUGCCCCGCAUCAGCGUGAUCAGCACCGGCCCCACCCUGCAGGC
CAGGAGGAGGCAGAGCGUGCUGAACCUGAUGACCCACAGCGUGAACCAG
GGCCAGAACAUCCACAGGAAGACCACCGCCAGCACCAGGAAGGUGAGCC
UGGCCCCACAGGCCAACCUGACCGAGCUGGACAUCUACAGCAGAAGGCU
GAGCCAGGAGACCGGCCUGGAGAUCAGCGAGGAGAUCAACGAGGAGGAC
CUGAAGGAGUGCUUCUUCGACGACAUGGAGAGCAUCCCAGCCGUGACCA
CCUGGAACACCUACCUGAGGUACAUCACCGUGCACAAGAGCCUGAUCUU
CGUGCUGAUCUGGUGCCUGGUGAUCUUCCUGGCCGAGGUGGCCGCCAGC
CUGGUGGUGCUGUGGCUGCUGGGCAACACCCCACUGCAGGACAAGGGCA
ACAGCACCCACAGCAGAAACAACAGCUACGCCGUGAUCAUCACCAGCAC
CAGCAGCUACUACGUGUUCUACAUCUACGUGGGAGUGGCCGACACCCUG
CUGGCCAUGGGCUUCUUCAGAGGCCUGCCACUGGUGCACACCCUGAUCA
CCGUGAGCAAGAUCCUGCACCACAAGAUGCUGCACAGCGUGCUGCAGGC
CCCCAUGAGCACCCUGAACACCCUGAAGGCCGGCGGGAUCCUGAACAGA
UUCAGCAAGGACAUCGCCAUCCUGGACGACCUGCUGCCCCUGACCAUCU
UCGACUUCAUCCAGCUGCUGCUGAUCGUGAUCGGAGCCAUCGCCGUGGU
GGCCGUGCUGCAGCCCUACAUCUUCGUGGCCACCGUGCCAGUGAUCGUG
GCCUUCAUCAUGCUGAGAGCCUACUUCCUGCAGACCAGCCAGCAGCUGA
AGCAGCUGGAGAGCGAGGGCAGGAGCCCAAUCUUCACCCACCUGGUGAC
CAGCCUGAAGGGACUGUGGACCCUGAGGGCCUUCGGCCGGCAGCCCUAC
UUCGAGACCCUGUUCCACAAGGCCCUGAACCUGCACACCGCCAACUGGU
UCCUGUACCUGAGCACCCUGCGCUGGUUCCAGAUGAGAAUCGAGAUGAU
CUUCGUGAUCUUCUUCAUCGCCGUGACCUUCAUCAGCAUCCUGACCACC
GGCGAGGGAGAGGGAAGAGUGGGCAUCAUCCUGACCCUGGCCAUGAACA
UCAUGAGCACCCUGCAGUGGGCCGUGAACAGCAGCAUCGACGUGGACAG
CCUGAUGAGGAGCGUGAGCAGGGUGUUCAAGUUCAUCGACAUGCCAACC
GAGGGCAAGCCCACCAAGAGCACCAAGCCAUACAAGAACGGCCAGCUGA
GCAAGGUGAUGAUCAUCGAGAACAGCCACGUGAAGAAGGACGACAUCUG
GCCCAGCGGCGGCCAGAUGACCGUGAAGGACCUGACCGCCAAGUACACC
GAGGGCGGCAACGCCAUCCUGGAGAACAUCAGCUUCAGCAUCAGCCCCG
GCCAGAGGGUGGGCCUGCUGGGAAGAACCGGCAGCGGCAAGAGCACCCU
GCUGAGCGCCUUCCUGAGACUGCUGAACACCGAGGGCGAGAUCCAGAUC
GACGGCGUGAGCUGGGACAGCAUCACCCUGCAGCAGUGGAGGAAGGCCU
UCGGCGUGAUCCCACAGAAGGUGUUCAUCUUCAGCGGAACCUUCAGAAA
GAACCUGGACCCCUACGAGCAGUGGAGCGACCAGGAGAUCUGGAAGGUG
GCCGACGAGGUGGGCCUGAGAAGCGUGAUCGAGCAGUUCCCCGGCAAGC
UGGACUUCGUGCUGGUGGACGGGGGCUGCGUGCUGAGCCACGGCCACAA
GCAGCUGAUGUGCCUGGCCAGAAGCGUGCUGAGCAAGGCCAAGAUCCUG
CUGCUGGACGAGCCCAGCGCCCACCUGGACCCAGUGACCUACCAGAUCA
UCAGAAGAACCCUGAAGCAGGCCUUCGCCGACUGCACCGUGAUCCUGUG
CGAGCACAGGAUCGAGGCCAUGCUGGAGUGCCAGCAGUUCCUGGUGAUC
GAGGAGAACAAGGUGCGGCAGUACGACAGCAUCCAGAAGCUGCUGAACG
AGAGGAGCCUGUUCCGGCAGGCCAUCAGCCCCAGCGACAGGGUGAAGCU
GUUCCCCCACCGGAACAGCAGCAAGUGCAAGAGCAAGCCCCAGAUCGCC
GCCCUGAAGGAGGAGACCGAGGAGGAGGUGCAGGACACCAGGCUGUAGA
UAAGUGAACUCGAGAUGAAGAUCCAGCCGGCCUUGGGAGCCUGGAGGAG
CAAAGACUGGGGUCUUUUGCGAAAGGGAUUGCAGGUUCAGAAGGCAUCU
UACCAUGGCUGGGGAAUUGUCUGGUGGUGGGGGGCAGGGGACAGAGGCC
AUGAAGGAGCAAGUUUUGUAUUUGUGACCUCAGCUUUGGGAAUAAAGGA
UCUUUUGAAGGCCAAUCUAG
(mARM2108)
SEQ ID NO: 80
GCAUGGGGAGGGGCGGCCCUCAAACGGGUCAUUGCCAUUAAUAGAGACC
UCAAACACCGCCUGCUAAAAAUACCCGACUGGAGGAGCAUAAAAGCGCA
GCCGAGCCCAGCGCCCCGCACUUUUCUGAGCAGACGUCCAGAGCAGAGU
CAGCCAGCCACCAUGCAGAGGAGCCCCCUGGAGAAGGCUAGCGUGGUGA
GCAAGCUGUUCUUCAGCUGGACCAGACCAAUCCUGAGGAAGGGCUACAG
ACAGCGCCUGGAGCUGAGCGACAUCUACCAGAUCCCCAGCGUGGACAGC
GCCGACAACCUGAGCGAGAAGCUGGAGAGAGAGUGGGACAGAGAGCUGG
CCAGCAAGAAGAACCCCAAGCUGAUCAACGCCCUGCGGAGGUGCUUCUU
CUGGAGAUUCAUGUUCUACGGAAUCUUCCUGUACCUGGGGGAGGUGACC
AAGGCCGUGCAGCCCCUGCUGCUGGGAAGAAUCAUCGCCAGCUACGACC
CCGACAACAAGGAGGAGCGCAGCAUCGCCAUCUACCUGGGCAUCGGCCU
GUGCCUGCUGUUCAUCGUGAGGACCCUGCUGCUGCACCCAGCCAUCUUC
GGCCUGCACCACAUCGGAAUGCAGAUGAGAAUCGCCAUGUUCAGCCUGA
UCUACAAGAAGACCCUGAAGCUGAGCAGCAGGGUGCUGGACAAGAUCAG
CAUCGGACAGCUGGUGAGCCUGCUGAGCAACAACCUGAACAAGUUCGAC
GAGGGACUGGCCCUGGCCCACUUCGUGUGGAUCGCCCCACUGCAGGUGG
CCCUGCUGAUGGGGCUGAUCUGGGAGCUGCUGCAGGCCAGCGCCUUCUG
CGGCCUGGGCUUCCUGAUCGUGCUGGCCCUGUUCCAGGCCGGCCUGGGC
AGAAUGAUGAUGAAGUACAGAGACCAGAGAGCCGGCAAGAUCAGCGAGA
GACUGGUGAUCACCAGCGAGAUGAUCGAGAACAUCCAGAGCGUGAAGGC
AUACUGCUGGGAGGAGGCCAUGGAGAAGAUGAUCGAGAACCUGAGACAG
ACCGAGCUGAAGCUGACCCGGAAGGCCGCCUACGUGAGAUACUUCAACA
GCAGCGCCUUCUUCUUCAGCGGGUUCUUCGUGGUGUUCCUGAGCGUGCU
GCCCUACGCCCUGAUCAAGGGCAUCAUCCUGCGGAAGAUCUUCACCACC
AUCAGCUUCUGCAUCGUGCUGCGCAUGGCCGUGACCCGGCAGUUCCCCU
GGGCCGUGCAGACCUGGUACGACAGCCUGGGAGCCAUCAACAAGAUCCA
GGACUUCCUGCAGAAGCAGGAGUACAAGACCCUGGAGUACAACCUGACC
ACCACCGAGGUGGUGAUGGAGAACGUGACCGCCUUCUGGGAGGAGGGAU
UCGGCGAGCUGUUCGAGAAGGCCAAGCAGAACAACAACAACAGAAAGAC
CAGCAACGGCGACGACAGCCUGUUCUUCAGCAACUUCAGCCUGCUGGGC
ACCCCCGUGCUGAAGGACAUCAACUUCAAGAUCGAGAGAGGACAGCUGC
UGGCCGUGGCCGGAAGCACCGGAGCCGGCAAGACCAGCCUGCUGAUGGU
GAUCAUGGGAGAGCUGGAGCCCAGCGAGGGCAAGAUCAAGCACAGCGGA
AGAAUCAGCUUCUGCAGCCAGUUCAGCUGGAUCAUGCCCGGCACCAUCA
AGGAGAACAUCAUCUUCGGCGUGAGCUACGACGAGUACAGAUACAGAAG
CGUGAUCAAGGCCUGCCAGCUGGAGGAGGACAUCAGCAAGUUCGCAGAG
AAGGACAACAUCGUGCUGGGAGAGGGCGGCAUCACCCUGAGCGGAGGCC
AGAGGGCCAGAAUCAGCCUGGCAAGAGCAGUGUACAAGGACGCCGACCU
GUACCUGCUGGACAGCCCCUUCGGAUACCUGGACGUGCUGACCGAGAAG
GAGAUCUUCGAGAGCUGCGUGUGCAAGCUGAUGGCCAACAAGACCAGGA
UCCUGGUGACCAGCAAGAUGGAGCACCUGAAGAAGGCCGACAAGAUCCU
GAUCCUGCACGAGGGCAGCAGCUACUUCUACGGGACCUUCAGCGAGCUG
CAGAACCUGCAGCCAGACUUCAGCAGCAAGCUGAUGGGCUGCGACAGCU
UCGACCAGUUCAGCGCCGAGAGAAGAAACAGCAUCCUGACCGAGACCCU
GCACAGGUUCAGCCUGGAGGGCGACGCCCCCGUGAGCUGGACCGAGACC
AAGAAGCAGAGCUUCAAGCAGACCGGAGAGUUCGGCGAGAAGAGGAAGA
ACAGCAUCCUGAACCCAAUCAACAGCAUCAGGAAGUUCAGCAUCGUGCA
GAAGACCCCACUGCAGAUGAACGGCAUCGAGGAGGACAGCGACGAGCCC
CUGGAGAGAAGGCUGAGCCUGGUGCCAGACAGCGAGCAGGGCGAGGCCA
UCCUGCCCCGCAUCAGCGUGAUCAGCACCGGCCCCACCCUGCAGGCCAG
GAGGAGGCAGAGCGUGCUGAACCUGAUGACCCACAGCGUGAACCAGGGC
CAGAACAUCCACAGGAAGACCACCGCCAGCACCAGGAAGGUGAGCCUGG
CCCCACAGGCCAACCUGACCGAGCUGGACAUCUACAGCAGAAGGCUGAG
CCAGGAGACCGGCCUGGAGAUCAGCGAGGAGAUCAACGAGGAGGACCUG
AAGGAGUGCUUCUUCGACGACAUGGAGAGCAUCCCAGCCGUGACCACCU
GGAACACCUACCUGAGGUACAUCACCGUGCACAAGAGCCUGAUCUUCGU
GCUGAUCUGGUGCCUGGUGAUCUUCCUGGCCGAGGUGGCCGCCAGCCUG
GUGGUGCUGUGGCUGCUGGGCAACACCCCACUGCAGGACAAGGGCAACA
GCACCCACAGCAGAAACAACAGCUACGCCGUGAUCAUCACCAGCACCAG
CAGCUACUACGUGUUCUACAUCUACGUGGGAGUGGCCGACACCCUGCUG
GCCAUGGGCUUCUUCAGAGGCCUGCCACUGGUGCACACCCUGAUCACCG
UGAGCAAGAUCCUGCACCACAAGAUGCUGCACAGCGUGCUGCAGGCCCC
CAUGAGCACCCUGAACACCCUGAAGGCCGGCGGGAUCCUGAACAGAUUC
AGCAAGGACAUCGCCAUCCUGGACGACCUGCUGCCCCUGACCAUCUUCG
ACUUCAUCCAGCUGCUGCUGAUCGUGAUCGGAGCCAUCGCCGUGGUGGC
CGUGCUGCAGCCCUACAUCUUCGUGGCCACCGUGCCAGUGAUCGUGGCC
UUCAUCAUGCUGAGAGCCUACUUCCUGCAGACCAGCCAGCAGCUGAAGC
AGCUGGAGAGCGAGGGCAGGAGCCCAAUCUUCACCCACCUGGUGACCAG
CCUGAAGGGACUGUGGACCCUGAGGGCCUUCGGCCGGCAGCCCUACUUC
GAGACCCUGUUCCACAAGGCCCUGAACCUGCACACCGCCAACUGGUUCC
UGUACCUGAGCACCCUGCGCUGGUUCCAGAUGAGAAUCGAGAUGAUCUU
CGUGAUCUUCUUCAUCGCCGUGACCUUCAUCAGCAUCCUGACCACCGGC
GAGGGAGAGGGAAGAGUGGGCAUCAUCCUGACCCUGGCCAUGAACAUCA
UGAGCACCCUGCAGUGGGCCGUGAACAGCAGCAUCGACGUGGACAGCCU
GAUGAGGAGCGUGAGCAGGGUGUUCAAGUUCAUCGACAUGCCAACCGAG
GGCAAGCCCACCAAGAGCACCAAGCCAUACAAGAACGGCCAGCUGAGCA
AGGUGAUGAUCAUCGAGAACAGCCACGUGAAGAAGGACGACAUCUGGCC
CAGCGGCGGCCAGAUGACCGUGAAGGACCUGACCGCCAAGUACACCGAG
GGCGGCAACGCCAUCCUGGAGAACAUCAGCUUCAGCAUCAGCCCCGGCC
AGAGGGUGGGCCUGCUGGGAAGAACCGGCAGCGGCAAGAGCACCCUGCU
GAGCGCCUUCCUGAGACUGCUGAACACCGAGGGCGAGAUCCAGAUCGAC
GGCGUGAGCUGGGACAGCAUCACCCUGCAGCAGUGGAGGAAGGCCUUCG
GCGUGAUCCCACAGAAGGUGUUCAUCUUCAGCGGAACCUUCAGAAAGAA
CCUGGACCCCUACGAGCAGUGGAGCGACCAGGAGAUCUGGAAGGUGGCC
GACGAGGUGGGCCUGAGAAGCGUGAUCGAGCAGUUCCCCGGCAAGCUGG
ACUUCGUGCUGGUGGACGGGGGCUGCGUGCUGAGCCACGGCCACAAGCA
GCUGAUGUGCCUGGCCAGAAGCGUGCUGAGCAAGGCCAAGAUCCUGCUG
CUGGACGAGCCCAGCGCCCACCUGGACCCAGUGACCUACCAGAUCAUCA
GAAGAACCCUGAAGCAGGCCUUCGCCGACUGCACCGUGAUCCUGUGCGA
GCACAGGAUCGAGGCCAUGCUGGAGUGCCAGCAGUUCCUGGUGAUCGAG
GAGAACAAGGUGCGGCAGUACGACAGCAUCCAGAAGCUGCUGAACGAGA
GGAGCCUGUUCCGGCAGGCCAUCAGCCCCAGCGACAGGGUGAAGCUGUU
CCCCCACCGGAACAGCAGCAAGUGCAAGAGCAAGCCCCAGAUCGCCGCC
CUGAAGGAGGAGACCGAGGAGGAGGUGCAGGACACCAGGCUGUAGAUAA
GUGAACUCGAGAGCCUUAGCCCGGAUGCCCACCCCUGCUGCCGCCACUG
GCUGUGCCUCCCCCGCCACCUGUGUGUUCUUUUGAUACAUUUAUCUUCU
GUUUUUCUCAAAUAAAGUUCAAAGCAACCACCUGUCAUCUAG
(mARM2109)
SEQ ID NO: 81
AGCAAAAGCAGGUAGAUAUUGAAAGCCACCAUGCAGAGGAGCCCCCUGG
AGAAGGCUAGCGUGGUGAGCAAGCUGUUCUUCAGCUGGACCAGACCAAU
CCUGAGGAAGGGCUACAGACAGCGCCUGGAGCUGAGCGACAUCUACCAG
AUCCCCAGCGUGGACAGCGCCGACAACCUGAGCGAGAAGCUGGAGAGAG
AGUGGGACAGAGAGCUGGCCAGCAAGAAGAACCCCAAGCUGAUCAACGC
CCUGCGGAGGUGCUUCUUCUGGAGAUUCAUGUUCUACGGAAUCUUCCUG
UACCUGGGGGAGGUGACCAAGGCCGUGCAGCCCCUGCUGCUGGGAAGAA
UCAUCGCCAGCUACGACCCCGACAACAAGGAGGAGCGCAGCAUCGCCAU
CUACCUGGGCAUCGGCCUGUGCCUGCUGUUCAUCGUGAGGACCCUGCUG
CUGCACCCAGCCAUCUUCGGCCUGCACCACAUCGGAAUGCAGAUGAGAA
UCGCCAUGUUCAGCCUGAUCUACAAGAAGACCCUGAAGCUGAGCAGCAG
GGUGCUGGACAAGAUCAGCAUCGGACAGCUGGUGAGCCUGCUGAGCAAC
AACCUGAACAAGUUCGACGAGGGACUGGCCCUGGCCCACUUCGUGUGGA
UCGCCCCACUGCAGGUGGCCCUGCUGAUGGGGCUGAUCUGGGAGCUGCU
GCAGGCCAGCGCCUUCUGCGGCCUGGGCUUCCUGAUCGUGCUGGCCCUG
UUCCAGGCCGGCCUGGGCAGAAUGAUGAUGAAGUACAGAGACCAGAGAG
CCGGCAAGAUCAGCGAGAGACUGGUGAUCACCAGCGAGAUGAUCGAGAA
CAUCCAGAGCGUGAAGGCAUACUGCUGGGAGGAGGCCAUGGAGAAGAUG
AUCGAGAACCUGAGACAGACCGAGCUGAAGCUGACCCGGAAGGCCGCCU
ACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUG
CGGAAGAUCUUCACCACCAUCAGCUUCUGCAUCGUGCUGCGCAUGGCCG
UGACCCGGCAGUUCCCCUGGGCCGUGCAGACCUGGUACGACAGCCUGGG
AGCCAUCAACAAGAUCCAGGACUUCCUGCAGAAGCAGGAGUACAAGACC
CUGGAGUACAACCUGACCACCACCGAGGUGGUGAUGGAGAACGUGACCG
CCUUCUGGGAGGAGGGAUUCGGCGAGCUGUUCGAGAAGGCCAAGCAGAA
CAACAACAACAGAAAGACCAGCAACGGCGACGACAGCCUGUUCUUCAGC
AACUUCAGCCUGCUGGGCACCCCCGUGCUGAAGGACAUCAACUUCAAGA
UCGAGAGAGGACAGCUGCUGGCCGUGGCCGGAAGCACCGGAGCCGGCAA
GACCAGCCUGCUGAUGGUGAUCAUGGGAGAGCUGGAGCCCAGCGAGGGC
AAGAUCAAGCACAGCGGAAGAAUCAGCUUCUGCAGCCAGUUCAGCUGGA
UCAUGCCCGGCACCAUCAAGGAGAACAUCAUCUUCGGCGUGAGCUACGA
CGAGUACAGAUACAGAAGCGUGAUCAAGGCCUGCCAGCUGGAGGAGGAC
AUCAGCAAGUUCGCAGAGAAGGACAACAUCGUGCUGGGAGAGGGCGGCA
UCACCCUGAGCGGAGGCCAGAGGGCCAGAAUCAGCCUGGCAAGAGCAGU
GUACAAGGACGCCGACCUGUACCUGCUGGACAGCCCCUUCGGAUACCUG
GACGUGCUGACCGAGAAGGAGAUCUUCGAGAGCUGCGUGUGCAAGCUGA
UGGCCAACAAGACCAGGAUCCUGGUGACCAGCAAGAUGGAGCACCUGAA
GAAGGCCGACAAGAUCCUGAUCCUGCACGAGGGCAGCAGCUACUUCUAC
GGGACCUUCAGCGAGCUGCAGAACCUGCAGCCAGACUUCAGCAGCAAGC
UGAUGGGCUGCGACAGCUUCGACCAGUUCAGCGCCGAGAGAAGAAACAG
CAUCCUGACCGAGACCCUGCACAGGUUCAGCCUGGAGGGCGACGCCCCC
GUGAGCUGGACCGAGACCAAGAAGCAGAGCUUCAAGCAGACCGGAGAGU
UCGGCGAGAAGAGGAAGAACAGCAUCCUGAACCCAAUCAACAGCAUCAG
GAAGUUCAGCAUCGUGCAGAAGACCCCACUGCAGAUGAACGGCAUCGAG
GAGGACAGCGACGAGCCCCUGGAGAGAAGGCUGAGCCUGGUGCCAGACA
GCGAGCAGGGCGAGGCCAUCCUGCCCCGCAUCAGCGUGAUCAGCACCGG
CCCCACCCUGCAGGCCAGGAGGAGGCAGAGCGUGCUGAACCUGAUGACC
CACAGCGUGAACCAGGGCCAGAACAUCCACAGGAAGACCACCGCCAGCA
CCAGGAAGGUGAGCCUGGCCCCACAGGCCAACCUGACCGAGCUGGACAU
CUACAGCAGAAGGCUGAGCCAGGAGACCGGCCUGGAGAUCAGCGAGGAG
AUCAACGAGGAGGACCUGAAGGAGUGCUUCUUCGACGACAUGGAGAGCA
UCCCAGCCGUGACCACCUGGAACACCUACCUGAGGUACAUCACCGUGCA
CAAGAGCCUGAUCUUCGUGCUGAUCUGGUGCCUGGUGAUCUUCCUGGCC
GAGGUGGCCGCCAGCCUGGUGGUGCUGUGGCUGCUGGGCAACACCCCAC
UGCAGGACAAGGGCAACAGCACCCACAGCAGAAACAACAGCUACGCCGU
GAUCAUCACCAGCACCAGCAGCUACUACGUGUUCUACAUCUACGUGGGA
GUGGCCGACACCCUGCUGGCCAUGGGCUUCUUCAGAGGCCUGCCACUGG
UGCACACCCUGAUCACCGUGAGCAAGAUCCUGCACCACAAGAUGCUGCA
CAGCGUGCUGCAGGCCCCCAUGAGCACCCUGAACACCCUGAAGGCCGGC
GGGAUCCUGAACAGAUUCAGCAAGGACAUCGCCAUCCUGGACGACCUGC
UGCCCCUGACCAUCUUCGACUUCAUCCAGCUGCUGCUGAUCGUGAUCGG
AGCCAUCGCCGUGGUGGCCGUGCUGCAGCCCUACAUCUUCGUGGCCACC
GUGCCAGUGAUCGUGGCCUUCAUCAUGCUGAGAGCCUACUUCCUGCAGA
CCAGCCAGCAGCUGAAGCAGCUGGAGAGCGAGGGCAGGAGCCCAAUCUU
CACCCACCUGGUGACCAGCCUGAAGGGACUGUGGACCCUGAGGGCCUUC
GGCCGGCAGCCCUACUUCGAGACCCUGUUCCACAAGGCCCUGAACCUGC
ACACCGCCAACUGGUUCCUGUACCUGAGCACCCUGCGCUGGUUCCAGAU
GAGAAUCGAGAUGAUCUUCGUGAUCUUCUUCAUCGCCGUGACCUUCAUC
AGCAUCCUGACCACCGGCGAGGGAGAGGGAAGAGUGGGCAUCAUCCUGA
CCCUGGCCAUGAACAUCAUGAGCACCCUGCAGUGGGCCGUGAACAGCAG
CAUCGACGUGGACAGCCUGAUGAGGAGCGUGAGCAGGGUGUUCAAGUUC
AUCGACAUGCCAACCGAGGGCAAGCCCACCAAGAGCACCAAGCCAUACA
AGAACGGCCAGCUGAGCAAGGUGAUGAUCAUCGAGAACAGCCACGUGAA
GAAGGACGACAUCUGGCCCAGCGGCGGCCAGAUGACCGUGAAGGACCUG
ACCGCCAAGUACACCGAGGGCGGCAACGCCAUCCUGGAGAACAUCAGCU
UCAGCAUCAGCCCCGGCCAGAGGGUGGGCCUGCUGGGAAGAACCGGCAG
CGGCAAGAGCACCCUGCUGAGCGCCUUCCUGAGACUGCUGAACACCGAG
GGCGAGAUCCAGAUCGACGGCGUGAGCUGGGACAGCAUCACCCUGCAGC
AGUGGAGGAAGGCCUUCGGCGUGAUCCCACAGAAGGUGUUCAUCUUCAG
CGGAACCUUCAGAAAGAACCUGGACCCCUACGAGCAGUGGAGCGACCAG
GAGAUCUGGAAGGUGGCCGACGAGGUGGGCCUGAGAAGCGUGAUCGAGC
AGUUCCCCGGCAAGCUGGACUUCGUGCUGGUGGACGGGGGCUGCGUGCU
GAGCCACGGCCACAAGCAGCUGAUGUGCCUGGCCAGAAGCGUGCUGAGC
AAGGCCAAGAUCCUGCUGCUGGACGAGCCCAGCGCCCACCUGGACCCAG
UGACCUACCAGAUCAUCAGAAGAACCCUGAAGCAGGCCUUCGCCGACUG
CACCGUGAUCCUGUGCGAGCACAGGAUCGAGGCCAUGCUGGAGUGCCAG
CAGUUCCUGGUGAUCGAGGAGAACAAGGUGCGGCAGUACGACAGCAUCC
AGAAGCUGCUGAACGAGAGGAGCCUGUUCCGGCAGGCCAUCAGCCCCAG
CGACAGGGUGAAGCUGUUCCCCCACCGGAACAGCAGCAAGUGCAAGAGC
AAGCCCCAGAUCGCCGCCCUGAAGGAGGAGACCGAGGAGGAGGUGCAGG
ACACCAGGCUGUAGAUAAGUGAACUCGAGCUAGUGACUGACUAGGAUCU
GGUUACCACUAAACCAGCCUCAAGAACACCCGAAUGGAGUCUCUAAGCU
ACAUAAUACCAACUUACACUUACAAAAUGUUGUCCCCCAAAAUGUAGCC
AUUCGUAUCUGCUCCUAAUAAAAAGAAAGUUUCUUCACAUUCUAG
(mARM2110)
SEQ ID NO: 82
AUUAUUACAUCAAAACAAAAAGCCGCCACCAUGCCCAGGAGCCCCCUGG
AGAAGGCUAGCGUGGUGAGCAAGCUGUUCUUCAGCUGGACCAGACCAAU
CCUGAGGAAGGGCUACAGACAGCGCCUGGAGCUGAGCGACAUCUACCAG
AUCCCCAGCGUGGACAGCGCCGACAACCUGAGCGAGAAGCUGGAGAGAG
AGUGGGACAGAGAGCUGGCCAGCAAGAAGAACCCCAAGCUGAUCAACGC
CCUGCGGAGGUGCUUCUUCUGGAGAUUCAUGUUCUACGGAAUCUUCCUG
UACCUGGGGGAGGUGACCAAGGCCGUGCAGCCCCUGCUGCUGGGAAGAA
UCAUCGCCAGCUACGACCCCGACAACAAGGAGGAGCGCAGCAUCGCCAU
CUACCUGGGCAUCGGCCUGUGCCUGCUGUUCAUCGUGAGGACCCUGCUG
CUGCACCCAGCCAUCUUCGGCCUGCACCACAUCGGAAUGCAGAUGAGAA
UCGCCAUGUUCAGCCUGAUCUACAAGAAGACCCUGAAGCUGAGCAGCAG
GGUGCUGGACAAGAUCAGCAUCGGACAGCUGGUGAGCCUGCUGAGCAAC
AACCUGAACAAGUUCGACGAGGGACUGGCCCUGGCCCACUUCGUGUGGA
UCGCCCCACUGCAGGUGGCCCUGCUGAUGGGGCUGAUCUGGGAGCUGCU
GCAGGCCAGCGCCUUCUGCGGCCUGGGCUUCCUGAUCGUGCUGGCCCUG
UUCCAGGCCGGCCUGGGCAGAAUGAUGAUGAAGUACAGAGACCAGAGAG
CCGGCAAGAUCAGCGAGAGACUGGUGAUCACCAGCGAGAUGAUCGAGAA
CAUCCAGAGCGUGAAGGCAUACUGCUGGGAGGAGGCCAUGGAGAAGAUG
AUCGAGAACCUGAGACAGACCGAGCUGAAGCUGACCCGGAAGGCCGCCU
ACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUG
CGGAAGAUCUUCACCACCAUCAGCUUCUGCAUCGUGCUGCGCAUGGCCG
UGACCCGGCAGUUCCCCUGGGCCGUGCAGACCUGGUACGACAGCCUGGG
AGCCAUCAACAAGAUCCAGGACUUCCUGCAGAAGCAGGAGUACAAGACC
CUGGAGUACAACCUGACCACCACCGAGGUGGUGAUGGAGAACGUGACCG
CCUUCUGGGAGGAGGGAUUCGGCGAGCUGUUCGAGAAGGCCAAGCAGAA
CAACAACAACAGAAAGACCAGCAACGGCGACGACAGCCUGUUCUUCAGC
AACUUCAGCCUGCUGGGCACCCCCGUGCUGAAGGACAUCAACUUCAAGA
UCGAGAGAGGACAGCUGCUGGCCGUGGCCGGAAGCACCGGAGCCGGCAA
GACCAGCCUGCUGAUGGUGAUCAUGGGAGAGCUGGAGCCCAGCGAGGGC
AAGAUCAAGCACAGCGGAAGAAUCAGCUUCUGCAGCCAGUUCAGCUGGA
UCAUGCCCGGCACCAUCAAGGAGAACAUCAUCUUCGGCGUGAGCUACGA
CGAGUACAGAUACAGAAGCGUGAUCAAGGCCUGCCAGCUGGAGGAGGAC
AUCAGCAAGUUCGCAGAGAAGGACAACAUCGUGCUGGGAGAGGGCGGCA
UCACCCUGAGCGGAGGCCAGAGGGCCAGAAUCAGCCUGGCAAGAGCAGU
GUACAAGGACGCCGACCUGUACCUGCUGGACAGCCCCUUCGGAUACCUG
GACGUGCUGACCGAGAAGGAGAUCUUCGAGAGCUGCGUGUGCAAGCUGA
UGGCCAACAAGACCAGGAUCCUGGUGACCAGCAAGAUGGAGCACCUGAA
GAAGGCCGACAAGAUCCUGAUCCUGCACGAGGGCAGCAGCUACUUCUAC
GGGACCUUCAGCGAGCUGCAGAACCUGCAGCCAGACUUCAGCAGCAAGC
UGAUGGGCUGCGACAGCUUCGACCAGUUCAGCGCCGAGAGAAGAAACAG
CAUCCUGACCGAGACCCUGCACAGGUUCAGCCUGGAGGGCGACGCCCCC
GUGAGCUGGACCGAGACCAAGAAGCAGAGCUUCAAGCAGACCGGAGAGU
UCGGCGAGAAGAGGAAGAACAGCAUCCUGAACCCAAUCAACAGCAUCAG
GAAGUUCAGCAUCGUGCAGAAGACCCCACUGCAGAUGAACGGCAUCGAG
GAGGACAGCGACGAGCCCCUGGAGAGAAGGCUGAGCCUGGUGCCAGACA
GCGAGCAGGGCGAGGCCAUCCUGCCCCGCAUCAGCGUGAUCAGCACCGG
CCCCACCCUGCAGGCCAGGAGGAGGCAGAGCGUGCUGAACCUGAUGACC
CACAGCGUGAACCAGGGCCAGAACAUCCACAGGAAGACCACCGCCAGCA
CCAGGAAGGUGAGCCUGGCCCCACAGGCCAACCUGACCGAGCUGGACAU
CUACAGCAGAAGGCUGAGCCAGGAGACCGGCCUGGAGAUCAGCGAGGAG
AUCAACGAGGAGGACCUGAAGGAGUGCUUCUUCGACGACAUGGAGAGCA
UCCCAGCCGUGACCACCUGGAACACCUACCUGAGGUACAUCACCGUGCA
CAAGAGCCUGAUCUUCGUGCUGAUCUGGUGCCUGGUGAUCUUCCUGGCC
GAGGUGGCCGCCAGCCUGGUGGUGCUGUGGCUGCUGGGCAACACCCCAC
UGCAGGACAAGGGCAACAGCACCCACAGCAGAAACAACAGCUACGCCGU
GAUCAUCACCAGCACCAGCAGCUACUACGUGUUCUACAUCUACGUGGGA
GUGGCCGACACCCUGCUGGCCAUGGGCUUCUUCAGAGGCCUGCCACUGG
UGCACACCCUGAUCACCGUGAGCAAGAUCCUGCACCACAAGAUGCUGCA
CAGCGUGCUGCAGGCCCCCAUGAGCACCCUGAACACCCUGAAGGCCGGC
GGGAUCCUGAACAGAUUCAGCAAGGACAUCGCCAUCCUGGACGACCUGC
UGCCCCUGACCAUCUUCGACUUCAUCCAGCUGCUGCUGAUCGUGAUCGG
AGCCAUCGCCGUGGUGGCCGUGCUGCAGCCCUACAUCUUCGUGGCCACC
GUGCCAGUGAUCGUGGCCUUCAUCAUGCUGAGAGCCUACUUCCUGCAGA
CCAGCCAGCAGCUGAAGCAGCUGGAGAGCGAGGGCAGGAGCCCAAUCUU
CACCCACCUGGUGACCAGCCUGAAGGGACUGUGGACCCUGAGGGCCUUC
GGCCGGCAGCCCUACUUCGAGACCCUGUUCCACAAGGCCCUGAACCUGC
ACACCGCCAACUGGUUCCUGUACCUGAGCACCCUGCGCUGGUUCCAGAU
GAGAAUCGAGAUGAUCUUCGUGAUCUUCUUCAUCGCCGUGACCUUCAUC
AGCAUCCUGACCACCGGCGAGGGAGAGGGAAGAGUGGGCAUCAUCCUGA
CCCUGGCCAUGAACAUCAUGAGCACCCUGCAGUGGGCCGUGAACAGCAG
CAUCGACGUGGACAGCCUGAUGAGGAGCGUGAGCAGGGUGUUCAAGUUC
AUCGACAUGCCAACCGAGGGCAAGCCCACCAAGAGCACCAAGCCAUACA
AGAACGGCCAGCUGAGCAAGGUGAUGAUCAUCGAGAACAGCCACGUGAA
GAAGGACGACAUCUGGCCCAGCGGCGGCCAGAUGACCGUGAAGGACCUG
ACCGCCAAGUACACCGAGGGCGGCAACGCCAUCCUGGAGAACAUCAGCU
UCAGCAUCAGCCCCGGCCAGAGGGUGGGCCUGCUGGGAAGAACCGGCAG
CGGCAAGAGCACCCUGCUGAGCGCCUUCCUGAGACUGCUGAACACCGAG
GGCGAGAUCCAGAUCGACGGCGUGAGCUGGGACAGCAUCACCCUGCAGC
AGUGGAGGAAGGCCUUCGGCGUGAUCCCACAGAAGGUGUUCAUCUUCAG
CGGAACCUUCAGAAAGAACCUGGACCCCUACGAGCAGUGGAGCGACCAG
GAGAUCUGGAAGGUGGCCGACGAGGUGGGCCUGAGAAGCGUGAUCGAGC
AGUUCCCCGGCAAGCUGGACUUCGUGCUGGUGGACGGGGGCUGCGUGCU
GAGCCACGGCCACAAGCAGCUGAUGUGCCUGGCCAGAAGCGUGCUGAGC
AAGGCCAAGAUCCUGCUGCUGGACGAGCCCAGCGCCCACCUGGACCCAG
UGACCUACCAGAUCAUCAGAAGAACCCUGAAGCAGGCCUUCGCCGACUG
CACCGUGAUCCUGUGCGAGCACAGGAUCGAGGCCAUGCUGGAGUGCCAG
CAGUUCCUGGUGAUCGAGGAGAACAAGGUGCGGCAGUACGACAGCAUCC
AGAAGCUGCUGAACGAGAGGAGCCUGUUCCGGCAGGCCAUCAGCCCCAG
CGACAGGGUGAAGCUGUUCCCCCACCGGAACAGCAGCAAGUGCAAGAGC
AAGCCCCAGAUCGCCGCCCUGAAGGAGGAGACCGAGGAGGAGGUGCAGG
ACACCAGGCUGUAGAUAAGUGAACUCGAGGCUGGAGCCUCGGUAGCCGU
UCCUCCUGCCCGCUGGGCCUCCCAACGGGCCCUCCUCCCCUCCUUGCAC
CGGCCCUUCCUGGUCUUUGAAUAAAGUCUGAGUGGGCAUCUAG
(mARM2111)
SEQ ID NO: 83
AUUAUUACAUCAAAACAAAAAGCCGCCACCAUGCCCAGGAGCCCCCUGG
AGAAGGCUAGCGUGGUGAGCAAGCUGUUCUUCAGCUGGACCAGACCAAU
CCUGAGGAAGGGCUACAGACAGCGCCUGGAGCUGAGCGACAUCUACCAG
AUCCCCAGCGUGGACAGCGCCGACAACCUGAGCGAGAAGCUGGAGAGAG
AGUGGGACAGAGAGCUGGCCAGCAAGAAGAACCCCAAGCUGAUCAACGC
CCUGCGGAGGUGCUUCUUCUGGAGAUUCAUGUUCUACGGAAUCUUCCUG
UACCUGGGGGAGGUGACCAAGGCCGUGCAGCCCCUGCUGCUGGGAAGAA
UCAUCGCCAGCUACGACCCCGACAACAAGGAGGAGCGCAGCAUCGCCAU
CUACCUGGGCAUCGGCCUGUGCCUGCUGUUCAUCGUGAGGACCCUGCUG
CUGCACCCAGCCAUCUUCGGCCUGCACCACAUCGGAAUGCAGAUGAGAA
UCGCCAUGUUCAGCCUGAUCUACAAGAAGACCCUGAAGCUGAGCAGCAG
GGUGCUGGACAAGAUCAGCAUCGGACAGCUGGUGAGCCUGCUGAGCAAC
AACCUGAACAAGUUCGACGAGGGACUGGCCCUGGCCCACUUCGUGUGGA
UCGCCCCACUGCAGGUGGCCCUGCUGAUGGGGCUGAUCUGGGAGCUGCU
GCAGGCCAGCGCCUUCUGCGGCCUGGGCUUCCUGAUCGUGCUGGCCCUG
UUCCAGGCCGGCCUGGGCAGAAUGAUGAUGAAGUACAGAGACCAGAGAG
CCGGCAAGAUCAGCGAGAGACUGGUGAUCACCAGCGAGAUGAUCGAGAA
CAUCCAGAGCGUGAAGGCAUACUGCUGGGAGGAGGCCAUGGAGAAGAUG
AUCGAGAACCUGAGACAGACCGAGCUGAAGCUGACCCGGAAGGCCGCCU
ACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUG
CGGAAGAUCUUCACCACCAUCAGCUUCUGCAUCGUGCUGCGCAUGGCCG
UGACCCGGCAGUUCCCCUGGGCCGUGCAGACCUGGUACGACAGCCUGGG
AGCCAUCAACAAGAUCCAGGACUUCCUGCAGAAGCAGGAGUACAAGACC
CUGGAGUACAACCUGACCACCACCGAGGUGGUGAUGGAGAACGUGACCG
CCUUCUGGGAGGAGGGAUUCGGCGAGCUGUUCGAGAAGGCCAAGCAGAA
CAACAACAACAGAAAGACCAGCAACGGCGACGACAGCCUGUUCUUCAGC
AACUUCAGCCUGCUGGGCACCCCCGUGCUGAAGGACAUCAACUUCAAGA
UCGAGAGAGGACAGCUGCUGGCCGUGGCCGGAAGCACCGGAGCCGGCAA
GACCAGCCUGCUGAUGGUGAUCAUGGGAGAGCUGGAGCCCAGCGAGGGC
AAGAUCAAGCACAGCGGAAGAAUCAGCUUCUGCAGCCAGUUCAGCUGGA
UCAUGCCCGGCACCAUCAAGGAGAACAUCAUCUUCGGCGUGAGCUACGA
CGAGUACAGAUACAGAAGCGUGAUCAAGGCCUGCCAGCUGGAGGAGGAC
AUCAGCAAGUUCGCAGAGAAGGACAACAUCGUGCUGGGAGAGGGCGGCA
UCACCCUGAGCGGAGGCCAGAGGGCCAGAAUCAGCCUGGCAAGAGCAGU
GUACAAGGACGCCGACCUGUACCUGCUGGACAGCCCCUUCGGAUACCUG
GACGUGCUGACCGAGAAGGAGAUCUUCGAGAGCUGCGUGUGCAAGCUGA
UGGCCAACAAGACCAGGAUCCUGGUGACCAGCAAGAUGGAGCACCUGAA
GAAGGCCGACAAGAUCCUGAUCCUGCACGAGGGCAGCAGCUACUUCUAC
GGGACCUUCAGCGAGCUGCAGAACCUGCAGCCAGACUUCAGCAGCAAGC
UGAUGGGCUGCGACAGCUUCGACCAGUUCAGCGCCGAGAGAAGAAACAG
CAUCCUGACCGAGACCCUGCACAGGUUCAGCCUGGAGGGCGACGCCCCC
GUGAGCUGGACCGAGACCAAGAAGCAGAGCUUCAAGCAGACCGGAGAGU
UCGGCGAGAAGAGGAAGAACAGCAUCCUGAACCCAAUCAACAGCAUCAG
GAAGUUCAGCAUCGUGCAGAAGACCCCACUGCAGAUGAACGGCAUCGAG
GAGGACAGCGACGAGCCCCUGGAGAGAAGGCUGAGCCUGGUGCCAGACA
GCGAGCAGGGCGAGGCCAUCCUGCCCCGCAUCAGCGUGAUCAGCACCGG
CCCCACCCUGCAGGCCAGGAGGAGGCAGAGCGUGCUGAACCUGAUGACC
CACAGCGUGAACCAGGGCCAGAACAUCCACAGGAAGACCACCGCCAGCA
CCAGGAAGGUGAGCCUGGCCCCACAGGCCAACCUGACCGAGCUGGACAU
CUACAGCAGAAGGCUGAGCCAGGAGACCGGCCUGGAGAUCAGCGAGGAG
AUCAACGAGGAGGACCUGAAGGAGUGCUUCUUCGACGACAUGGAGAGCA
UCCCAGCCGUGACCACCUGGAACACCUACCUGAGGUACAUCACCGUGCA
CAAGAGCCUGAUCUUCGUGCUGAUCUGGUGCCUGGUGAUCUUCCUGGCC
GAGGUGGCCGCCAGCCUGGUGGUGCUGUGGCUGCUGGGCAACACCCCAC
UGCAGGACAAGGGCAACAGCACCCACAGCAGAAACAACAGCUACGCCGU
GAUCAUCACCAGCACCAGCAGCUACUACGUGUUCUACAUCUACGUGGGA
GUGGCCGACACCCUGCUGGCCAUGGGCUUCUUCAGAGGCCUGCCACUGG
UGCACACCCUGAUCACCGUGAGCAAGAUCCUGCACCACAAGAUGCUGCA
CAGCGUGCUGCAGGCCCCCAUGAGCACCCUGAACACCCUGAAGGCCGGC
GGGAUCCUGAACAGAUUCAGCAAGGACAUCGCCAUCCUGGACGACCUGC
UGCCCCUGACCAUCUUCGACUUCAUCCAGCUGCUGCUGAUCGUGAUCGG
AGCCAUCGCCGUGGUGGCCGUGCUGCAGCCCUACAUCUUCGUGGCCACC
GUGCCAGUGAUCGUGGCCUUCAUCAUGCUGAGAGCCUACUUCCUGCAGA
CCAGCCAGCAGCUGAAGCAGCUGGAGAGCGAGGGCAGGAGCCCAAUCUU
CACCCACCUGGUGACCAGCCUGAAGGGACUGUGGACCCUGAGGGCCUUC
GGCCGGCAGCCCUACUUCGAGACCCUGUUCCACAAGGCCCUGAACCUGC
ACACCGCCAACUGGUUCCUGUACCUGAGCACCCUGCGCUGGUUCCAGAU
GAGAAUCGAGAUGAUCUUCGUGAUCUUCUUCAUCGCCGUGACCUUCAUC
AGCAUCCUGACCACCGGCGAGGGAGAGGGAAGAGUGGGCAUCAUCCUGA
CCCUGGCCAUGAACAUCAUGAGCACCCUGCAGUGGGCCGUGAACAGCAG
CAUCGACGUGGACAGCCUGAUGAGGAGCGUGAGCAGGGUGUUCAAGUUC
AUCGACAUGCCAACCGAGGGCAAGCCCACCAAGAGCACCAAGCCAUACA
AGAACGGCCAGCUGAGCAAGGUGAUGAUCAUCGAGAACAGCCACGUGAA
GAAGGACGACAUCUGGCCCAGCGGCGGCCAGAUGACCGUGAAGGACCUG
ACCGCCAAGUACACCGAGGGCGGCAACGCCAUCCUGGAGAACAUCAGCU
UCAGCAUCAGCCCCGGCCAGAGGGUGGGCCUGCUGGGAAGAACCGGCAG
CGGCAAGAGCACCCUGCUGAGCGCCUUCCUGAGACUGCUGAACACCGAG
GGCGAGAUCCAGAUCGACGGCGUGAGCUGGGACAGCAUCACCCUGCAGC
AGUGGAGGAAGGCCUUCGGCGUGAUCCCACAGAGAGUGUUCAUCUUCAG
CGGAACCUUCAGAAAGAACCUGGACCCCUACGAGCAGUGGAGCGACCAG
GAGAUCUGGAAGGUGGCCGACGAGGUGGGCCUGAGAAGCGUGAUCGAGC
AGUUCCCCGGCAAGCUGGACUUCGUGCUGGUGGACGGGGGCUGCGUGCU
GAGCCACGGCCACAAGCAGCUGAUGUGCCUGGCCAGAAGCGUGCUGAGC
AAGGCCAAGAUCCUGCUGCUGGACGAGCCCAGCGCCCACCUGGACCCAG
UGACCUACCAGAUCAUCAGAAGAACCCUGAAGCAGGCCUUCGCCGACUG
CACCGUGAUCCUGUGCGAGCACAGGAUCGAGGCCAUGCUGGAGUGCCAG
CAGUUCCUGGUGAUCGAGGAGAACAAGGUGCGGCAGUACGACAGCAUCC
AGAAGCUGCUGAACGAGAGGAGCCUGUUCCGGCAGGCCAUCAGCCCCAG
CGACAGGGUGAAGCUGUUCCCCCACCGGAACAGCAGCAAGUGCAAGAGC
AAGCCCCAGAUCGCCGCCCUGAAGGAGGAGACCGAGGAGGAGGUGCAGG
ACACCAGGCUGUAGAUAAGUGAACUCGAGGCUGGAGCCUCGGUAGCCGU
UCCUCCUGCCCGCUGGGCCUCCCAACGGGCCCUCCUCCCCUCCUUGCAC
CGGCCCUUCCUGGUCUUUGAAUAAAGUCUGAGUGGGCAUCUAG
(mARM2268)
SEQ ID NO: 84
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCUACCCCUACGACGUGCCCGACUACGCCACCG
GCGAGGGAGAGGGAAGAGUGGGCAUCAUCCUGACCCUGGCCAUGAACAU
CAUGAGCACCCUGCAGUGGGCUGUGAACUCCAGCAUCGACGUGGACAGC
CUGAUGAGGUCUGUGAGCAGGGUGUUCAAGUUCAUCGACAUGCCAACCG
AGGGCAAGCCUACCAAGAGCACCAAGCCAUACAAGAACGGCCAGCUGAG
CAAGGUGAUGAUCAUCGAGAACAGCCACGUGAAGAAGGACGACAUCUGG
CCCAGCGGCGGCCAGAUGACCGUGAAGGACCUGACCGCCAAGUACACCG
AGGGCGGCAACGCCAUCCUGGAGAACAUCUCCUUCUCAAUCAGCCCUGG
CCAGAGGGUGGGCCUGCUGGGAAGAACCGGCUCAGGCAAGAGCACCCUG
CUGAGCGCCUUCCUGAGACUGCUGAACACCGAGGGCGAGAUCCAGAUCG
ACGGCGUGUCUUGGGACUCAAUCACCCUGCAGCAGUGGAGGAAGGCCUU
CGGCGUGAUCCCACAGAAGGUGUUCAUCUUCUCUGGAACCUUCAGAAAG
AACCUGGACCCCUACGAGCAGUGGAGCGACCAGGAGAUCUGGAAGGUGG
CCGACGAGGUGGGCCUGAGAUCUGUGAUCGAGCAGUUCCCUGGCAAGCU
GGACUUCGUGCUGGUGGACGGGGGCUGCGUGCUGAGCCACGGCCACAAG
CAGCUGAUGUGCCUGGCCAGAUCUGUGCUGAGCAAGGCCAAGAUCCUGC
UGCUGGACGAGCCCAGUGCCCACCUGGACCCAGUGACCUACCAGAUCAU
CAGAAGAACCCUGAAGCAGGCCUUCGCCGACUGCACCGUGAUCCUGUGC
GAGCACAGGAUCGAGGCCAUGCUGGAGUGCCAGCAGUUCCUGGUGAUCG
AGGAGAACAAGGUGCGGCAGUACGACUCCAUCCAGAAGCUGCUGAACGA
GAGGAGCCUGUUCCGGCAGGCCAUCAGCCCCUCCGACAGGGUGAAGCUG
UUCCCCCACCGGAACAGCAGCAAGUGCAAGUCUAAGCCCCAGAUCGCCG
CCCUGAAGGAGGAGACCGAGGAGGAGGUGCAGGACACCAGGCUGUAGAU
AAGUGAACUCGAGCUAGUGACUGACUAGGAUCUGGUUACCACUAAACCA
GCCUCAAGAACACCCGAAUGGAGUCUCUAAGCUACAUAAUACCAACUUA
CACUUACAAAAUGUUGUCCCCCAAAAUGUAGCCAUUCGUAUCUGCUCCU
AAUAAAAAGAAAGUUUCUUCACAUUCUAG
(mARM2269)
SEQ ID NO: 85
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGUACCCCUACGACGUGCCCGACUACGCCUAGAU
AAGUGAACUCGAGCUAGUGACUGACUAGGAUCUGGUUACCACUAAACCA
GCCUCAAGAACACCCGAAUGGAGUCUCUAAGCUACAUAAUACCAACUUA
CACUUACAAAAUGUUGUCCCCCAAAAUGUAGCCAUUCGUAUCUGCUCCU
AAUAAAAAGAAAGUUUCUUCACAUUCUAG
(mARM2381)
SEQ ID NO: 86
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGGACUACAAGGACGAUGACGAUAAGUAGAUAAG
UGAACUCGAGCUAGUGACUGACUAGGAUCUGGUUACCACUAAACCAGCC
UCAAGAACACCCGAAUGGAGUCUCUAAGCUACAUAAUACCAACUUACAC
UUACAAAAUGUUGUCCCCCAAAAUGUAGCCAUUCGUAUCUGCUCCUAAU
AAAAAGAAAGUUUCUUCACAUUCUAG
(mARM2382)
SEQ ID NO: 87
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGGGCGGCGGCAGCGGCGAGCAGAAACUGAUCAG
CGAAGAGGAUCUGAACGGCGGCGGCAGCGGCGAGCAGAAACUGAUCAGC
GAAGAGGAUCUGAACGGCGGCGGCAGCGGCGAGCAGAAACUGAUCAGCG
AAGAGGAUCUGAACUAGAUAAGUGAACUCGAGCUAGUGACUGACUAGGA
UCUGGUUACCACUAAACCAGCCUCAAGAACACCCGAAUGGAGUCUCUAA
GCUACAUAAUACCAACUUACACUUACAAAAUGUUGUCCCCCAAAAUGUA
GCCAUUCGUAUCUGCUCCUAAUAAAAAGAAAGUUUCUUCACAUUCUAG
(mARM2383)
SEQ ID NO: 88
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGGUGAGCGGCUGGCGGCUGUUCAAGAAGAUUAG
CUAGAUAAGUGAACUCGAGCUAGUGACUGACUAGGAUCUGGUUACCACU
AAACCAGCCUCAAGAACACCCGAAUGGAGUCUCUAAGCUACAUAAUACC
AACUUACACUUACAAAAUGUUGUCCCCCAAAAUGUAGCCAUUCGUAUCU
GCUCCUAAUAAAAAGAAAGUUUCUUCACAUUCUAG
(mARM2384)
SEQ ID NO: 89
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGGGCGGCGGCGGCAGCGGCGGCAGCAGCGUGAG
CAAGGGCGAGGAGCUGUUCACCGGCGUGGUGCCCAUCCUGGUGGAGCUG
GACGGCGACGUGAACGGCCACAAGUUCAGCGUGAGCGGCGAGGGCGAGG
GCGACGCCACCUACGGCAAGCUGACCCUGAAGUUCAUCUGCACCACCGG
CAAGCUGCCCGUGCCCUGGCCCACCCUGGUGACCACCCUGACCUACGGC
GUGCAGUGCUUCAGCAGGUACCCCGACCACAUGAAGCAGCACGACUUCU
UCAAGAGCGCCAUGCCCGAGGGCUACGUGCAGGAGAGGACCAUCUUCUU
CAAGGACGACGGCAACUACAAGACCAGGGCCGAGGUGAAGUUCGAGGGC
GACACCCUGGUGAACAGGAUCGAGCUGAAGGGCAUCGACUUCAAGGAGG
ACGGCAACAUCCUGGGCCACAAGCUGGAGUACAACUACAACAGCCACAA
CGUGUACAUCAUGGCCGACAAGCAGAAGAACGGCAUCAAGGUGAACUUC
AAGAUCAGGCACAACAUCGAGGACGGCAGCGUGCAGCUGGCCGACCACU
ACCAGCAGAACACCCCCAUCGGCGACGGCCCCGUGCUGCUGCCCGACAA
CCACUACCUGAGCACCCAGAGCGCCCUGAGCAAGGACCCCAACGAGAAG
AGGGACCACAUGGUGCUGCUGGAGUUCGUGACCGCCGCCGGCAUCACCC
UGGGCAUGGACGAGCUGUACAAGUAGAUAAGUGAACUCGAGCUAGUGAC
UGACUAGGAUCUGGUUACCACUAAACCAGCCUCAAGAACACCCGAAUGG
AGUCUCUAAGCUACAUAAUACCAACUUACACUUACAAAAUGUUGUCCCC
CAAAAUGUAGCCAUUCGUAUCUGCUCCUAAUAAAAAGAAAGUUUCUUCA
CAUUCUAG
(mARM2491)
SEQ ID NO: 90
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAAAAGGCCAGCGUUGUCUCCAAACUUUUUUUCAGCUGGACCAG
ACCAAUUUUGAGGAAAGGAUACAGACAGCGCCUGGAAUUGUCAGACAUA
UACCAAAUCCCUUCUGUUGAUUCUGCUGACAAUCUAUCUGAAAAAUUGG
AAAGAGAAUGGGAUAGAGAGCUGGCUUCAAAGAAAAAUCCUAAACUCAU
UAAUGCCCUUCGGCGAUGUUUUUUCUGGAGAUUUAUGUUCUAUGGAAUC
UUUUUAUAUUUAGGGGAAGUCACCAAAGCAGUACAGCCUCUCUUACUGG
GAAGAAUCAUAGCUUCCUAUGACCCGGAUAACAAGGAGGAACGCUCUAU
CGCGAUUUAUCUAGGCAUAGGCUUAUGCCUUCUCUUUAUUGUGAGGACA
CUGCUCCUACACCCAGCCAUUUUUGGCCUUCAUCACAUUGGAAUGCAGA
UGAGAAUAGCUAUGUUUAGUUUGAUUUAUAAGAAGACUUUAAAGCUGUC
AAGCCGUGUUCUAGAUAAAAUAAGUAUUGGACAACUUGUUAGUCUCCUU
UCCAACAACCUGAACAAAUUUGAUGAAGGACUUGCAUUGGCACAUUUCG
UGUGGAUCGCUCCUUUGCAAGUGGCACUCCUCAUGGGGCUAAUCUGGGA
GUUGUUACAGGCGUCUGCCUUCUGUGGACUUGGUUUCCUGAUAGUCCUU
GCCCUUUUUCAGGCUGGGCUAGGGAGAAUGAUGAUGAAGUACAGAGAUC
AGAGAGCUGGGAAGAUCAGUGAAAGACUCGUAAUUACCUCAGAAAUGAU
UGAGAACAUCCAAUCUGUUAAGGCAUACUGCUGGGAAGAAGCAAUGGAA
AAAAUGAUUGAAAACUUAAGACAAACAGAACUGAAACUGACUCGGAAGG
CAGCCUAUGUGAGAUACUUCAAUAGCUCAGCCUUCUUCUUCUCAGGGUU
CUUUGUGGUGUUUUUAUCUGUGCUUCCCUAUGCACUAAUCAAAGGAAUC
AUCCUCCGGAAAAUAUUCACCACCAUCUCAUUCUGCAUUGUUCUGCGCA
UGGCGGUCACUCGGCAAUUUCCCUGGGCUGUACAAACAUGGUAUGACUC
UCUUGGAGCAAUAAACAAAAUACAGGAUUUCUUACAAAAGCAAGAAUAU
AAGACAUUGGAAUAUAACUUAACGACUACAGAAGUAGUGAUGGAGAAUG
UAACAGCCUUCUGGGAGGAGGGAUUUGGGGAAUUAUUUGAGAAAGCAAA
ACAAAACAAUAACAAUAGAAAAACUUCUAAUGGUGAUGACAGCCUCUUC
UUCAGUAAUUUCUCACUUCUUGGUACUCCUGUCCUGAAAGAUAUUAAUU
UCAAGAUAGAAAGAGGACAGUUGUUGGCGGUUGCUGGAUCCACUGGAGC
AGGCAAGACUUCACUUCUAAUGGUGAUUAUGGGAGAACUGGAGCCUUCA
GAGGGUAAAAUUAAGCACAGUGGAAGAAUUUCAUUCUGUUCUCAGUUUU
CCUGGAUUAUGCCUGGCACCAUUAAAGAAAAUAUCAUCUUUGGUGUUUC
CUAUGAUGAAUAUAGAUACAGAAGCGUCAUCAAAGCAUGCCAACUAGAA
GAGGACAUCUCCAAGUUUGCAGAGAAAGACAAUAUAGUUCUUGGAGAAG
GUGGAAUCACACUGAGUGGAGGUCAACGAGCAAGAAUUUCUUUAGCAAG
AGCAGUAUACAAAGAUGCUGAUUUGUAUUUAUUAGACUCUCCUUUUGGA
UACCUAGAUGUUUUAACAGAAAAAGAAAUAUUUGAAAGCUGUGUCUGUA
AACUGAUGGCUAACAAAACUAGGAUUUUGGUCACUUCUAAAAUGGAACA
UUUAAAGAAAGCUGACAAAAUAUUAAUUUUGCAUGAAGGUAGCAGCUAU
UUUUAUGGGACAUUUUCAGAACUCCAAAAUCUACAGCCAGACUUUAGCU
CAAAACUCAUGGGAUGUGAUUCUUUCGACCAAUUUAGUGCAGAAAGAAG
AAAUUCAAUCCUAACUGAGACAUUACACCGUUUCUCAUUAGAAGGAGAU
GCUCCUGUCUCCUGGACAGAAACAAAAAAACAAUCUUUUAAACAGACUG
GAGAGUUUGGGGAAAAAAGGAAGAAUUCUAUUCUCAAUCCAAUCAACUC
UAUACGAAAAUUUUCCAUUGUGCAAAAGACUCCCUUACAAAUGAAUGGC
AUCGAAGAGGAUUCUGAUGAGCCUUUAGAGAGAAGGCUGUCCUUAGUAC
CAGAUUCUGAGCAGGGAGAGGCGAUACUGCCUCGCAUCAGCGUGAUCAG
CACUGGCCCCACGCUUCAGGCACGAAGGAGGCAGUCUGUCCUGAACCUG
AUGACACACUCAGUUAACCAAGGUCAGAACAUUCACCGAAAGACAACAG
CAUCCACACGAAAAGUGUCACUGGCCCCUCAGGCAAACUUGACUGAACU
GGAUAUAUAUUCAAGAAGGUUAUCUCAAGAAACUGGCUUGGAAAUAAGU
GAAGAAAUUAACGAAGAAGACUUAAAGGAGUGCUUUUUUGAUGAUAUGG
AGAGCAUACCAGCAGUGACUACAUGGAACACAUACCUUCGAUAUAUUAC
UGUCCACAAGAGCUUAAUUUUUGUGCUAAUUUGGUGCUUAGUAAUUUUU
CUGGCAGAGGUGGCUGCUUCUUUGGUUGUGCUGUGGCUCCUUGGAAACA
CUCCUCUUCAAGACAAAGGGAAUAGUACUCAUAGUAGAAAUAACAGCUA
UGCAGUGAUUAUCACCAGCACCAGUUCGUAUUAUGUGUUUUACAUUUAC
GUGGGAGUAGCCGACACUUUGCUUGCUAUGGGAUUCUUCAGAGGUCUAC
CACUGGUGCAUACUCUAAUCACAGUGUCGAAAAUUUUACACCACAAAAU
GUUACAUUCUGUUCUUCAAGCACCUAUGUCAACCCUCAACACGUUGAAA
GCAGGUGGGAUUCUUAAUAGAUUCUCCAAAGAUAUAGCAAUUUUGGAUG
ACCUUCUGCCUCUUACCAUAUUUGACUUCAUCCAGUUGUUAUUAAUUGU
GAUUGGAGCUAUAGCAGUUGUCGCAGUUUUACAACCCUACAUCUUUGUU
GCAACAGUGCCAGUGAUAGUGGCUUUUAUUAUGUUGAGAGCAUAUUUCC
UCCAAACCUCACAGCAACUCAAACAACUGGAAUCUGAAGGCAGGAGUCC
AAUUUUCACUCAUCUUGUUACAAGCUUAAAAGGACUAUGGACACUUCGU
GCCUUCGGACGGCAGCCUUACUUUGAAACUCUGUUCCACAAAGCUCUGA
AUUUACAUACUGCCAACUGGUUCUUGUACCUGUCAACACUGCGCUGGUU
CCAAAUGAGAAUAGAAAUGAUUUUUGUCAUCUUCUUCAUUGCUGUUACC
UUCAUUUCCAUUUUAACAACAGGAGAAGGAGAAGGAAGAGUUGGUAUUA
UCCUGACUUUAGCCAUGAAUAUCAUGAGUACAUUGCAGUGGGCUGUAAA
CUCCAGCAUAGAUGUGGAUAGCUUGAUGCGAUCUGUGAGCCGAGUCUUU
AAGUUCAUUGACAUGCCAACAGAAGGUAAACCUACCAAGUCAACCAAAC
CAUACAAGAAUGGCCAACUCUCGAAAGUUAUGAUUAUUGAGAAUUCACA
CGUGAAGAAAGAUGACAUCUGGCCCUCAGGGGGCCAAAUGACUGUCAAA
GAUCUCACAGCAAAAUACACAGAAGGUGGAAAUGCCAUAUUAGAGAACA
UUUCCUUCUCAAUAAGUCCUGGCCAGAGGGUGGGCCUCUUGGGAAGAAC
UGGAUCAGGGAAGAGUACUUUGUUAUCAGCUUUUUUGAGACUACUGAAC
ACUGAAGGAGAAAUCCAGAUCGAUGGUGUGUCUUGGGAUUCAAUAACUU
UGCAACAGUGGAGGAAAGCCUUUGGAGUGAUACCACAGAAAGUAUUUAU
UUUUUCUGGAACAUUUAGAAAAAACUUGGAUCCCUAUGAACAGUGGAGU
GAUCAAGAAAUAUGGAAAGUUGCAGAUGAGGUUGGGCUCAGAUCUGUGA
UAGAACAGUUUCCUGGGAAGCUUGACUUUGUCCUUGUGGAUGGGGGCUG
UGUCCUAAGCCAUGGCCACAAGCAGUUGAUGUGCUUGGCUAGAUCUGUU
CUCAGUAAGGCGAAGAUCUUGCUGCUUGAUGAACCCAGUGCUCAUUUGG
AUCCAGUAACAUACCAAAUAAUUAGAAGAACUCUAAAACAAGCAUUUGC
UGAUUGCACAGUAAUUCUCUGUGAACACAGGAUAGAAGCAAUGCUGGAA
UGCCAACAAUUUUUGGUCAUAGAAGAGAACAAAGUGCGGCAGUACGAUU
CCAUCCAGAAACUGCUGAACGAGAGGAGCCUCUUCCGGCAAGCCAUCAG
CCCCUCCGACAGGGUGAAGCUCUUUCCCCACCGGAACUCAAGCAAGUGC
AAGUCUAAGCCCCAGAUUGCUGCUCUGAAAGAGGAGACAGAAGAAGAGG
UGCAAGAUACAAGGCUUUAGCUCGAGCUAGUGACUGACUAGGAUCUGGU
UACCACUAAACCAGCCUCAAGAACACCCGAAUGGAGUCUCUAAGCUACA
UAAUACCAACUUACACUUACAAAAUGUUGUCCCCCAAAAUGUAGCCAUU
CGUAUCUGCUCCUAAUAAAAAGAAAGUUUCUUCACAUUCUAG
(mARM2492)
SEQ ID NO: 91
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGUCGC
CUCUGGAGAAGGCCAGCGUGGUGUCCAAGCUGUUCUUCAGCUGGACCAG
ACCAAUCCUGAGGAAGGGCUACAGACAGCGCCUGGAGCUGUCAGACAUC
UACCAGAUCCCUUCUGUGGACUCUGCUGACAACCUGUCUGAGAAGCUGG
AGAGAGAGUGGGACAGAGAGCUGGCCAGCAAGAAGAACCCUAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCUCUGCUGCUGG
GAAGAAUCAUCGCCUCCUACGACCCCGACAACAAGGAGGAGCGCUCUAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGUC
AAGCAGGGUGCUGGACAAGAUCAGUAUCGGACAGCUGGUGAGUCUGCUG
UCCAACAACCUGAACAAGUUCGACGAGGGACUGGCCCUGGCCCACUUCG
UGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUG
GCCCUGUUCCAGGCCGGCCUGGGCAGAAUGAUGAUGAAGUACAGAGACC
AGAGAGCUGGCAAGAUCAGCGAGAGACUGGUGAUCACCUCAGAGAUGAU
CGAGAACAUCCAGUCUGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCUCAGGGUU
CUUCGUGGUGUUCCUGUCUGUGCUGCCCUACGCCCUGAUCAAGGGCAUC
AUCCUGCGGAAGAUCUUCACCACCAUCUCAUUCUGCAUCGUGCUGCGCA
UGGCCGUGACCCGGCAGUUCCCCUGGGCCGUGCAGACCUGGUACGACUC
UCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCUCUAACGGCGACGACAGCCUGUUC
UUCAGCAACUUCAGCCUGCUGGGCACCCCUGUGCUGAAGGACAUCAACU
UCAAGAUCGAGAGAGGACAGCUGCUGGCCGUGGCCGGAUCCACCGGAGC
CGGCAAGACCUCACUGCUGAUGGUGAUCAUGGGAGAGCUGGAGCCUUCA
GAGGGCAAGAUCAAGCACAGUGGAAGAAUCUCAUUCUGCUCUCAGUUCU
CCUGGAUCAUGCCUGGCACCAUCAAGGAGAACAUCAUCUUCGGUGUGUC
CUACGACGAGUACAGAUACAGAAGCGUGAUCAAGGCCUGCCAGCUGGAG
GAGGACAUCUCCAAGUUCGCAGAGAAGGACAACAUCGUGCUGGGAGAGG
GUGGCAUCACCCUGAGCGGAGGCCAGAGGGCCAGAAUCUCUCUGGCAAG
AGCAGUGUACAAGGACGCUGACCUGUACCUGCUGGACUCUCCUUUCGGA
UACCUGGACGUGCUGACCGAGAAGGAGAUCUUCGAGAGCUGCGUGUGCA
AGCUGAUGGCCAACAAGACCAGGAUCCUGGUGACCUCUAAGAUGGAGCA
CCUGAAGAAGGCUGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACUCUUUCGACCAGUUCAGCGCCGAGAGAAG
AAACAGCAUCCUGACCGAGACCCUGCACAGGUUCAGCCUGGAGGGCGAC
GCCCCUGUGUCCUGGACCGAGACCAAGAAGCAGUCUUUCAAGCAGACCG
GAGAGUUCGGCGAGAAGAGGAAGAACUCUAUCCUGAACCCAAUCAACUC
UAUCAGGAAGUUCUCCAUCGUGCAGAAGACCCCCCUGCAGAUGAACGGC
AUCGAGGAGGACUCUGACGAGCCUCUGGAGAGAAGGCUGUCCCUGGUGC
CAGACUCUGAGCAGGGCGAGGCCAUCCUGCCUCGCAUCAGCGUGAUCAG
CACCGGCCCCACCCUGCAGGCCAGGAGGAGGCAGUCUGUGCUGAACCUG
AUGACCCACAGCGUGAACCAGGGCCAGAACAUCCACAGGAAGACCACCG
CCUCCACCAGGAAGGUGAGCCUGGCCCCUCAGGCCAACCUGACCGAGCU
GGACAUCUACAGCAGAAGGCUGUCUCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCUCUCUGGUGGUGCUGUGGCUGCUGGGCAACA
CCCCUCUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGUCUGC
CACUGGUGCACACCCUGAUCACCGUGAGCAAGAUCCUGCACCACAAGAU
GCUGCACUCUGUGCUGCAGGCCCCUAUGAGCACCCUGAACACCCUGAAG
GCCGGUGGGAUCCUGAACAGAUUCUCCAAGGACAUCGCCAUCCUGGACG
ACCUGCUGCCUCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCC
AAUCUUCACCCACCUGGUGACCAGCCUGAAGGGACUGUGGACCCUGAGG
GCCUUCGGCCGGCAGCCUUACUUCGAGACCCUGUUCCACAAGGCUCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCUCAGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG
(mARM2493)
SEQ ID NO: 92
UCAACACAACAUAUACAAAACAAACGAAUCUCAAGCAAUCAAGCAUUCU
ACUUCUAUUGCAGCAAUUUAAAUCAUUUCUUUUAAAGCAAAAGCAAUUU
UCUGAAAAUUUUCACCAUUUACGAACGAUAGCCACCAUGCAGAGGAGCC
CCCUGGAGAAGGCUAGCGUGGUGAGCAAGCUGUUCUUCAGCUGGACCAG
ACCAAUCCUGAGGAAGGGCUACAGACAGCGCCUGGAGCUGAGCGACAUC
UACCAGAUCCCCAGCGUGGACAGCGCCGACAACCUGAGCGAGAAGCUGG
AGAGAGAGUGGGACAGAGAGCUGGCCAGCAAGAAGAACCCCAAGCUGAU
CAACGCCCUGCGGAGGUGCUUCUUCUGGAGAUUCAUGUUCUACGGAAUC
UUCCUGUACCUGGGGGAGGUGACCAAGGCCGUGCAGCCCCUGCUGCUGG
GAAGAAUCAUCGCCAGCUACGACCCCGACAACAAGGAGGAGCGCAGCAU
CGCCAUCUACCUGGGCAUCGGCCUGUGCCUGCUGUUCAUCGUGAGGACC
CUGCUGCUGCACCCAGCCAUCUUCGGCCUGCACCACAUCGGAAUGCAGA
UGAGAAUCGCCAUGUUCAGCCUGAUCUACAAGAAGACCCUGAAGCUGAG
CAGCAGGGUGCUGGACAAGAUCAGCAUCGGACAGCUGGUGAGCCUGCUG
AGCAACAACCUGAACAAGUUCGACGAGGGACUGGCCCUGGCCCACUUCG
UGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUG
GCCCUGUUCCAGGCCGGCCUGGGCAGAAUGAUGAUGAAGUACAGAGACC
AGAGAGCCGGCAAGAUCAGCGAGAGACUGGUGAUCACCAGCGAGAUGAU
CGAGAACAUCCAGAGCGUGAAGGCAUACUGCUGGGAGGAGGCCAUGGAG
AAGAUGAUCGAGAACCUGAGACAGACCGAGCUGAAGCUGACCCGGAAGG
CCGCCUACGUGAGAUACUUCAACAGCAGCGCCUUCUUCUUCAGCGGGUU
CUUCGUGGUGUUCCUGAGCGUGCUGCCCUACGCCCUGAUCAAGGGCAUC
AUCCUGCGGAAGAUCUUCACCACCAUCAGCUUCUGCAUCGUGCUGCGCA
UGGCCGUGACCCGGCAGUUCCCCUGGGCCGUGCAGACCUGGUACGACAG
CCUGGGAGCCAUCAACAAGAUCCAGGACUUCCUGCAGAAGCAGGAGUAC
AAGACCCUGGAGUACAACCUGACCACCACCGAGGUGGUGAUGGAGAACG
UGACCGCCUUCUGGGAGGAGGGAUUCGGCGAGCUGUUCGAGAAGGCCAA
GCAGAACAACAACAACAGAAAGACCAGCAACGGCGACGACAGCCUGUUC
UUCAGCAACUUCAGCCUGCUGGGCACCCCCGUGCUGAAGGACAUCAACU
UCAAGAUCGAGAGAGGACAGCUGCUGGCCGUGGCCGGAAGCACCGGAGC
CGGCAAGACCAGCCUGCUGAUGGUGAUCAUGGGAGAGCUGGAGCCCAGC
GAGGGCAAGAUCAAGCACAGCGGAAGAAUCAGCUUCUGCAGCCAGUUCA
GCUGGAUCAUGCCCGGCACCAUCAAGGAGAACAUCAUCUUCGGCGUGAG
CUACGACGAGUACAGAUACAGAAGCGUGAUCAAGGCCUGCCAGCUGGAG
GAGGACAUCAGCAAGUUCGCAGAGAAGGACAACAUCGUGCUGGGAGAGG
GCGGCAUCACCCUGAGCGGAGGCCAGAGGGCCAGAAUCAGCCUGGCAAG
AGCAGUGUACAAGGACGCCGACCUGUACCUGCUGGACAGCCCCUUCGGA
UACCUGGACGUGCUGACCGAGAAGGAGAUCUUCGAGAGCUGCGUGUGCA
AGCUGAUGGCCAACAAGACCAGGAUCCUGGUGACCAGCAAGAUGGAGCA
CCUGAAGAAGGCCGACAAGAUCCUGAUCCUGCACGAGGGCAGCAGCUAC
UUCUACGGGACCUUCAGCGAGCUGCAGAACCUGCAGCCAGACUUCAGCA
GCAAGCUGAUGGGCUGCGACAGCUUCGACCAGUUCAGCGCCGAGAGAAG
AAACAGCAUCCUGACCGAGACCCUGCACAGGUUCAGCCUGGAGGGCGAC
GCCCCCGUGAGCUGGACCGAGACCAAGAAGCAGAGCUUCAAGCAGACCG
GAGAGUUCGGCGAGAAGAGGAAGAACAGCAUCCUGAACCCAAUCAACAG
CAUCAGGAAGUUCAGCAUCGUGCAGAAGACCCCACUGCAGAUGAACGGC
AUCGAGGAGGACAGCGACGAGCCCCUGGAGAGAAGGCUGAGCCUGGUGC
CAGACAGCGAGCAGGGCGAGGCCAUCCUGCCCCGCAUCAGCGUGAUCAG
CACCGGCCCCACCCUGCAGGCCAGGAGGAGGCAGAGCGUGCUGAACCUG
AUGACCCACAGCGUGAACCAGGGCCAGAACAUCCACAGGAAGACCACCG
CCAGCACCAGGAAGGUGAGCCUGGCCCCACAGGCCAACCUGACCGAGCU
GGACAUCUACAGCAGAAGGCUGAGCCAGGAGACCGGCCUGGAGAUCAGC
GAGGAGAUCAACGAGGAGGACCUGAAGGAGUGCUUCUUCGACGACAUGG
AGAGCAUCCCAGCCGUGACCACCUGGAACACCUACCUGAGGUACAUCAC
CGUGCACAAGAGCCUGAUCUUCGUGCUGAUCUGGUGCCUGGUGAUCUUC
CUGGCCGAGGUGGCCGCCAGCCUGGUGGUGCUGUGGCUGCUGGGCAACA
CCCCACUGCAGGACAAGGGCAACAGCACCCACAGCAGAAACAACAGCUA
CGCCGUGAUCAUCACCAGCACCAGCAGCUACUACGUGUUCUACAUCUAC
GUGGGAGUGGCCGACACCCUGCUGGCCAUGGGCUUCUUCAGAGGCCUGC
CACUGGUGCACACCCUGAUCACCGUGAGCAAGAUCCUGCACCACAAGAU
GCUGCACAGCGUGCUGCAGGCCCCCAUGAGCACCCUGAACACCCUGAAG
GCCGGCGGGAUCCUGAACAGAUUCAGCAAGGACAUCGCCAUCCUGGACG
ACCUGCUGCCCCUGACCAUCUUCGACUUCAUCCAGCUGCUGCUGAUCGU
GAUCGGAGCCAUCGCCGUGGUGGCCGUGCUGCAGCCCUACAUCUUCGUG
GCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCC
AAUCUUCACCCACCUGGUGACCAGCCUGAAGGGACUGUGGACCCUGAGG
GCCUUCGGCCGGCAGCCCUACUUCGAGACCCUGUUCCACAAGGCCCUGA
ACCUGCACACCGCCAACUGGUUCCUGUACCUGAGCACCCUGCGCUGGUU
CCAGAUGAGAAUCGAGAUGAUCUUCGUGAUCUUCUUCAUCGCCGUGACC
UUCAUCUCCAUCCUGACCACCGGCGAGGGAGAGGGAAGAGUGGGCAUCA
UCCUGACCCUGGCCAUGAACAUCAUGAGCACCCUGCAGUGGGCUGUGAA
CUCCAGCAUCGACGUGGACAGCCUGAUGAGGUCUGUGAGCAGGGUGUUC
AAGUUCAUCGACAUGCCAACCGAGGGCAAGCCUACCAAGAGCACCAAGC
CAUACAAGAACGGCCAGCUGAGCAAGGUGAUGAUCAUCGAGAACAGCCA
CGUGAAGAAGGACGACAUCUGGCCCAGCGGCGGCCAGAUGACCGUGAAG
GACCUGACCGCCAAGUACACCGAGGGCGGCAACGCCAUCCUGGAGAACA
UCUCCUUCUCAAUCAGCCCUGGCCAGAGGGUGGGCCUGCUGGGAAGAAC
CGGCAGCGGCAAGAGCACCCUGCUGAGCGCCUUCCUGAGACUGCUGAAC
ACCGAGGGCGAGAUCCAGAUCGACGGCGUGUCUUGGGACUCAAUCACCC
UGCAGCAGUGGAGGAAGGCCUUCGGCGUGAUCCCACAGAAGGUGUUCAU
CUUCUCUGGAACCUUCAGAAAGAACCUGGACCCCUACGAGCAGUGGAGC
GACCAGGAGAUCUGGAAGGUGGCCGACGAGGUGGGCCUGAGAUCUGUGA
UCGAGCAGUUCCCUGGCAAGCUGGACUUCGUGCUGGUGGACGGGGGCUG
CGUGCUGAGCCACGGCCACAAGCAGCUGAUGUGCCUGGCCAGAUCUGUG
CUGAGCAAGGCCAAGAUCCUGCUGCUGGACGAGCCCAGUGCCCACCUGG
ACCCAGUGACCUACCAGAUCAUCAGAAGAACCCUGAAGCAGGCCUUCGC
CGACUGCACCGUGAUCCUGUGCGAGCACAGGAUCGAGGCCAUGCUGGAG
UGCCAGCAGUUCCUGGUGAUCGAGGAGAACAAGGUGCGGCAGUACGACU
CCAUCCAGAAGCUGCUGAACGAGAGGAGCCUGUUCCGGCAGGCCAUCAG
CCCCUCCGACAGGGUGAAGCUGUUCCCCCACCGGAACAGCAGCAAGUGC
AAGUCUAAGCCCCAGAUCGCCGCCCUGAAGGAGGAGACCGAGGAGGAGG
UGCAGGACACCAGGCUGUAGAUAAGUGAACUCGAGCUAGUGACUGACUA
GGAUCUGGUUACCACUAAACCAGCCUCAAGAACACCCGAAUGGAGUCUC
UAAGCUACAUAAUACCAACUUACACUUACAAAAUGUUGUCCCCCAAAAU
GUAGCCAUUCGUAUCUGCUCCUAAUAAAAAGAAAGUUUCUUCACAUUCU
AG

### Peptide Sequences

(Natural hCFTR)
SEQ ID NO: 93
MQRSPLEKAS VVSKLFFSWT RPILRKGYRQ RLELSDIYQI PSVDSADNLS EKLEREWDRE
LASKKNPKLI NALRRCFFWR FMFYGIFLYL GEVTKAVQPL LLGRIIASYD PDNKEERSIA
IYLGIGLCLL FIVRTLLLHP AIFGLHHIGM QMRIAMFSLI YKKTLKLSSR VLDKISIGQL
VSLLSNNLNK FDEGLALAHF VWIAPLQVAL LMGLIWELLQ ASAFCGLGFL IVLALFQAGL
GRMMMKYRDQ RAGKISERLV ITSEMIENIQ SVKAYCWEEA MEKMIENLRQ TELKLTRKAA
YVRYFNSSAF FFSGFFVVFL SVLPYALIKG IILRKIFTTI SFCIVLRMAV TRQFPWAVQT
WYDSLGAINK IQDFLQKQEY KTLEYNLTTT EVVMENVTAF WEEGFGELFE KAKQNNNNRK
TSNGDDSLFF SNFSLLGTPV LKDINFKIER GQLLAVAGST GAGKTSLLMV IMGELEPSEG
KIKHSGRISF CSQFSWIMPG TIKENIIFGV SYDEYRYRSV IKACQLEEDI SKFAEKDNIV
LGEGGITLSG GQRARISLAR AVYKDADLYL LDSPFGYLDV LTEKEIFESC VCKLMANKTR
ILVTSKMEHL KKADKILILH EGSSYFYGTF SELQNLQPDF SSKLMGCDSF DQFSAERRNS
ILTETLHRFS LEGDAPVSWT ETKKQSFKQT GEFGEKRKNS ILNPINSIRK FSIVQKTPLQ
MNGIEEDSDE PLERRLSLVP DSEQGEAILP RISVISTGPT LQARRRQSVL NLMTHSVNQG
QNIHRKTTAS TRKVSLAPQA NLTELDIYSR RLSQETGLEI SEEINEEDLK ECFFDDMESI
PAVTTWNTYL RYITVHKSLI FVLIWCLVIF LAEVAASLVV LWLLGNTPLQ DKGNSTHSRN
NSYAVIITST SSYYVFYIYV GVADTLLAMG FFRGLPLVHT LITVSKILHH KMLHSVLQAP
MSTLNTLKAG GILNRFSKDI AILDDLLPLT IFDFIQLLLI VIGAIAVVAV LQPYIFVATV
PVIVAFIMLR AYFLQTSQQL KQLESEGRSP IFTHLVTSLK GLWTLRAFGR QPYFETLFHK
ALNLHTANWF LYLSTLRWFQ MRIEMIFVIF FIAVTFISIL TTGEGEGRVG IILTLAMNIM
STLQWAVNSS IDVDSLMRSV SRVFKFIDMP TEGKPTKSTK PYKNGQLSKV MIIENSHVKK
DDIWPSGGQM TVKDLTAKYT EGGNAILENI SFSISPGQRV GLLGRTGSGK STLLSAFLRL
LNTEGEIQID GVSWDSITLQ QWRKAFGVIP QKVFIFSGTF RKNLDPYEQW SDQEIWKVAD
EVGLRSVIEQ FPGKLDFVLV DGGCVLSHGH KQLMCLARSV LSKAKILLLD EPSAHLDPVT
YQIIRRTLKQ AFADCTVILC EHRIEAMLEC QQFLVIEENK VRQYDSIQKL LNERSLFRQA
ISPSDRVKLF PHRNSSKCKS KPQIAALKEE TEEEVQDTRL
(pARM764)
SEQ ID NO: 94
MQRSPLEKASVVSKLFFSWTRPILRKGYRQRLELSDIYQIPSVDSADNLSEKLEREWDRELAS
KKNPKLINALRRCFFWRFMFYGIFLYLGEVTKAVQPLLLGRIIASYDPDNKEERSIAIYLGIG
LCLLFIVRTLLLHPAIFGLHHIGMQMRIAMFSLIYKKTLKLSSRVLDKISIGQLVSLLSNNLN
KFDEGLALAHFVWIAPLQVALLMGLIWELLQASAFCGLGFLIVLALFQAGLGRMMMKYRDQRA
GKISERLVITSEMIENIQSVKAYCWEEAMEKMIENLRQTELKLTRKAAYVRYFNSSAFFFSGF
FVVFLSVLPYALIKGIILRKIFTTISFCIVLRMAVTRQFPWAVQTWYDSLGAINKIQDFLQKQ
EYKTLEYNLTTTEVVMENVTAFWEEGFGELFEKAKQNNNNRKTSNGDDSLFFSNFSLLGTPVL
KDINFKIERGQLLAVAGSTGAGKTSLLMVIMGELEPSEGKIKHSGRISFCSQFSWIMPGTIKE
NIIFGVSYDEYRYRSVIKACQLEEDISKFAEKDNIVLGEGGITLSGGQRARISLARAVYKDAD
LYLLDSPFGYLDVLTEKEIFESCVCKLMANKTRILVTSKMEHLKKADKILILHEGSSYFYGTF
SELQNLQPDFSSKLMGCDSFDQFSAERRNSILTETLHRFSLEGDAPVSWTETKKQSFKQTGEF
GEKRKNSILNPINSIRKFSIVQKTPLQMNGIEEDSDEPLERRLSLVPDSEQGEAILPRISVIS
TGPTLQARRRQSVLNLMTHSVNQGQNIHRKTTASTRKVSLAPQANLTELDIYSRRLSQETGLE
ISEEINEEDLKECFFDDMESIPAVTTWNTYLRYITVHKSLIFVLIWCLVIFLAEVAASLVVLW
LLGNTPLQDKGNSTHSRNNSYAVIITSTSSYYVFYIYVGVADTLLAMGFFRGLPLVHTLITVS
KILHHKMLHSVLQAPMSTLNTLKAGGILNRFSKDIAILDDLLPLTIFDFIQLLLIVIGAIAVV
AVLQPYIFVATVPVIVAFIMLRAYFLQTSQQLKQLESEGRSPIFTHLVTSLKGLWTLRAFGRQ
PYFETLFHKALNLHTANWFLYLSTLRWFQMRIEMIFVIFFIAVTFISILTTGEGEGRVGIILT
LAMNIMSTLQWAVNSSIDVDSLMRSVSRVFKFIDMPTEGKPTKSTKPYKNGQLSKVMIIENSH
VKKDDIWPSGGQMTVKDLTAKYTEGGNAILENISFSISPGQRVGLLGRTGSGKSTLLSAFLRL
LNTEGEIQIDGVSWDSITLQQWRKAFGVIPQKVFIFSGTFRKNLDPYEQWSDQEIWKVADEVG
LRSVIEQFPGKLDFVLVDGGCVLSHGHKQLMCLARSVLSKAKILLLDEPSAHLDPVTYQIIRR
TLKQAFADCTVILCEHRIEAMLECQQFLVIEENKVRQYDSIQKLLNERSLFRQAISPSDRVKL
FPHRNSSKCKSKPQIAALKEETEEEVQDTRL*
(pARM1880)
SEQ ID NO: 95
MGQRSPLEKASVVSKLFFSWTRPILRKGYRQRLELSDIYQIPSVDSADNLSEKLEREWDRELA
SKKNPKLINALRRCFFWRFMFYGIFLYLGEVTKAVQPLLLGRIIASYDPDNKEERSIAIYLGI
GLCLLFIVRTLLLHPAIFGLHHIGMQMRIAMFSLIYKKTLKLSSRVLDKISIGQLVSLLSNNL
NKFDEGLALAHFVWIAPLQVALLMGLIWELLQASAFCGLGFLIVLALFQAGLGRMMMKYRDQR
AGKISERLVITSEMIENIQSVKAYCWEEAMEKMIENLRQTELKLTRKAAYVRYFNSSAFFFSG
FFVVFLSVLPYALIKGIILRKIFTTISFCIVLRMAVTRQFPWAVQTWYDSLGAINKIQDFLQK
QEYKTLEYNLTTTEVVMENVTAFWEEGFGELFEKAKQNNNNRKTSNGDDSLFFSNFSLLGTPV
LKDINFKIERGQLLAVAGSTGAGKTSLLMVIMGELEPSEGKIKHSGRISFCSQFSWIMPGTIK
ENIIFGVSYDEYRYRSVIKACQLEEDISKFAEKDNIVLGEGGITLSGGQRARISLARAVYKDA
DLYLLDSPFGYLDVLTEKEIFESCVCKLMANKTRILVTSKMEHLKKADKILILHEGSSYFYGT
FSELQNLQPDFSSKLMGCDSFDQFSAERRNSILTETLHRFSLEGDAPVSWTETKKQSFKQTGE
FGEKRKNSILNPINSIRKFSIVQKTPLQMNGIEEDSDEPLERRLSLVPDSEQGEAILPRISVI
STGPTLQARRRQSVLNLMTHSVNQGQNIHRKTTASTRKVSLAPQANLTELDIYSRRLSQETGL
EISEEINEEDLKECFFDDMESIPAVTTWNTYLRYITVHKSLIFVLIWCLVIFLAEVAASLVVL
WLLGNTPLQDKGNSTHSRNNSYAVIITSTSSYYVFYIYVGVADTLLAMGFFRGLPLVHTLITV
SKILHHKMLHSVLQAPMSTLNTLKAGGILNRFSKDIAILDDLLPLTIFDFIQLLLIVIGAIAV
VAVLQPYIFVATVPVIVAFIMLRAYFLQTSQQLKQLESEGRSPIFTHLVTSLKGLWTLRAFGR
QPYFETLFHKALNLHTANWFLYLSTLRWFQMRIEMIFVIFFIAVTFISILTTGEGEGRVGIIL
TLAMNIMSTLQWAVNSSIDVDSLMRSVSRVFKFIDMPTEGKPTKSTKPYKNGQLSKVMIIENS
HVKKDDIWPSGGQMTVKDLTAKYTEGGNAILENISFSISPGQRVGLLGRTGSGKSTLLSAFLR
LLNTEGEIQIDGVSWDSITLQQWRKAFGVIPQKVFIFSGTFRKNLDPYEQWSDQEIWKVADEV
GLRSVIEQFPGKLDFVLVDGGCVLSHGHKQLMCLARSVLSKAKILLLDEPSAHLDPVTYQIIR
RTLKQAFADCTVILCEHRIEAMLECQQFLVIEENKVRQYDSIQKLLNERSLFRQAISPSDRVK
LFPHRNSSKCKSKPQIAALKEETEEEVQDTRL*
(pARM2110)
SEQ ID NO: 96
MPRSPLEKASVVSKLFFSWTRPILRKGYRQRLELSDIYQIPSVDSADNLSEKLEREWDRELAS
KKNPKLINALRRCFFWRFMFYGIFLYLGEVTKAVQPLLLGRIIASYDPDNKEERSIAIYLGIG
LCLLFIVRTLLLHPAIFGLHHIGMQMRIAMFSLIYKKTLKLSSRVLDKISIGQLVSLLSNNLN
KFDEGLALAHFVWIAPLQVALLMGLIWELLQASAFCGLGFLIVLALFQAGLGRMMMKYRDQRA
GKISERLVITSEMIENIQSVKAYCWEEAMEKMIENLRQTELKLTRKAAYVRYFNSSAFFFSGF
FVVFLSVLPYALIKGIILRKIFTTISFCIVLRMAVTRQFPWAVQTWYDSLGAINKIQDFLQKQ
EYKTLEYNLTTTEVVMENVTAFWEEGFGELFEKAKQNNNNRKTSNGDDSLFFSNFSLLGTPVL
KDINFKIERGQLLAVAGSTGAGKTSLLMVIMGELEPSEGKIKHSGRISFCSQFSWIMPGTIKE
NIIFGVSYDEYRYRSVIKACQLEEDISKFAEKDNIVLGEGGITLSGGQRARISLARAVYKDAD
LYLLDSPFGYLDVLTEKEIFESCVCKLMANKTRILVTSKMEHLKKADKILILHEGSSYFYGTF
SELQNLQPDFSSKLMGCDSFDQFSAERRNSILTETLHRFSLEGDAPVSWTETKKQSFKQTGEF
GEKRKNSILNPINSIRKFSIVQKTPLQMNGIEEDSDEPLERRLSLVPDSEQGEAILPRISVIS
TGPTLQARRRQSVLNLMTHSVNQGQNIHRKTTASTRKVSLAPQANLTELDIYSRRLSQETGLE
ISEEINEEDLKECFFDDMESIPAVTTWNTYLRYITVHKSLIFVLIWCLVIFLAEVAASLVVLW
LLGNTPLQDKGNSTHSRNNSYAVIITSTSSYYVFYIYVGVADTLLAMGFFRGLPLVHTLITVS
KILHHKMLHSVLQAPMSTLNTLKAGGILNRFSKDIAILDDLLPLTIFDFIQLLLIVIGAIAVV
AVLQPYIFVATVPVIVAFIMLRAYFLQTSQQLKQLESEGRSPIFTHLVTSLKGLWTLRAFGRQ
PYFETLFHKALNLHTANWFLYLSTLRWFQMRIEMIFVIFFIAVTFISILTTGEGEGRVGIILT
LAMNIMSTLQWAVNSSIDVDSLMRSVSRVFKFIDMPTEGKPTKSTKPYKNGQLSKVMIIENSH
VKKDDIWPSGGQMTVKDLTAKYTEGGNAILENISFSISPGQRVGLLGRTGSGKSTLLSAFLRL
LNTEGEIQIDGVSWDSITLQQWRKAFGVIPQKVFIFSGTFRKNLDPYEQWSDQEIWKVADEVG
LRSVIEQFPGKLDFVLVDGGCVLSHGHKQLMCLARSVLSKAKILLLDEPSAHLDPVTYQIIRR
TLKQAFADCTVILCEHRIEAMLECQQFLVIEENKVRQYDSIQKLLNERSLFRQAISPSDRVKL
FPHRNSSKCKSKPQIAALKEETEEEVQDTRL*
(pARM2111)
SEQ ID NO: 97
MPRSPLEKASVVSKLFFSWTRPILRKGYRQRLELSDIYQIPSVDSADNLSEKLEREWDRELAS
KKNPKLINALRRCFFWRFMFYGIFLYLGEVTKAVQPLLLGRIIASYDPDNKEERSIAIYLGIG
LCLLFIVRTLLLHPAIFGLHHIGMQMRIAMFSLIYKKTLKLSSRVLDKISIGQLVSLLSNNLN
KFDEGLALAHFVWIAPLQVALLMGLIWELLQASAFCGLGFLIVLALFQAGLGRMMMKYRDQRA
GKISERLVITSEMIENIQSVKAYCWEEAMEKMIENLRQTELKLTRKAAYVRYFNSSAFFFSGF
FVVFLSVLPYALIKGIILRKIFTTISFCIVLRMAVTRQFPWAVQTWYDSLGAINKIQDFLQKQ
EYKTLEYNLTTTEVVMENVTAFWEEGFGELFEKAKQNNNNRKTSNGDDSLFFSNFSLLGTPVL
KDINFKIERGQLLAVAGSTGAGKTSLLMVIMGELEPSEGKIKHSGRISFCSQFSWIMPGTIKE
NIIFGVSYDEYRYRSVIKACQLEEDISKFAEKDNIVLGEGGITLSGGQRARISLARAVYKDAD
LYLLDSPFGYLDVLTEKEIFESCVCKLMANKTRILVTSKMEHLKKADKILILHEGSSYFYGTF
SELQNLQPDFSSKLMGCDSFDQFSAERRNSILTETLHRFSLEGDAPVSWTETKKQSFKQTGEF
GEKRKNSILNPINSIRKFSIVQKTPLQMNGIEEDSDEPLERRLSLVPDSEQGEAILPRISVIS
TGPTLQARRRQSVLNLMTHSVNQGQNIHRKTTASTRKVSLAPQANLTELDIYSRRLSQETGLE
ISEEINEEDLKECFFDDMESIPAVTTWNTYLRYITVHKSLIFVLIWCLVIFLAEVAASLVVLW
LLGNTPLQDKGNSTHSRNNSYAVIITSTSSYYVFYIYVGVADTLLAMGFFRGLPLVHTLITVS
KILHHKMLHSVLQAPMSTLNTLKAGGILNRFSKDIAILDDLLPLTIFDFIQLLLIVIGAIAVV
AVLQPYIFVATVPVIVAFIMLRAYFLQTSQQLKQLESEGRSPIFTHLVTSLKGLWTLRAFGRQ
PYFETLFHKALNLHTANWFLYLSTLRWFQMRIEMIFVIFFIAVTFISILTTGEGEGRVGIILT
LAMNIMSTLQWAVNSSIDVDSLMRSVSRVFKFIDMPTEGKPTKSTKPYKNGQLSKVMIIENSH
VKKDDIWPSGGQMTVKDLTAKYTEGGNAILENISFSISPGQRVGLLGRTGSGKSTLLSAFLRL
LNTEGEIQIDGVSWDSITLQQWRKAFGVIPQRVFIFSGTFRKNLDPYEQWSDQEIWKVADEVG
LRSVIEQFPGKLDFVLVDGGCVLSHGHKQLMCLARSVLSKAKILLLDEPSAHLDPVTYQIIRR
TLKQAFADCTVILCEHRIEAMLECQQFLVIEENKVRQYDSIQKLLNERSLFRQAISPSDRVKL
FPHRNSSKCKSKPQIAALKEETEEEVQDTRL*
(pARM1835)
SEQ ID NO: 98
MQRSPLEKASVVSKLFFSWTRPILRKGYRQRLELSDIYQIPSVDSADNLSEKLEREWDRELAS
KKNPKLINALRRCFFWRFMFYGIFLYLGEVTKAVQPLLLGRIIASYDPDNKEERSIAIYLGIG
LCLLFIVRTLLLHPAIFGLHHIGMQMRIAMFSLIYKKTLKLSSRVLDKISIGQLVSLLSNNLN
KFDEGLALAHFVWIAPLQVALLMGLIWELLQASAFCGLGFLIVLALFQAGLGRMMMKYRDQRA
GKISERLVITSEMIENIQSVKAYCWEEAMEKMIENLRQTELKLTRKAAYVRYFNSSAFFFSGF
FVVFLSVLPYALIKGIILRKIFTTISFCIVLRMAVTRQFPWAVQTWYDSLGAINKIQDFLQKQ
EYKTLEYNLTTTEVVMENVTAFWEEGFGELFEKAKQNNNNRKTSNGDDSLFFSNFSLLGTPVL
KDINFKIERGQLLAVAGSTGAGKTSLLMVIMGELEPSEGKIKHSGRISFCSQFSWIMPGTIKE
NIIFGVSYDEYRYRSVIKACQLEEDISKFAEKDNIVLGEGGITLSGGQRARISLARAVYKDAD
LYLLDSPFGYLDVLTEKEIFESCVCKLMANKTRILVTSKMEHLKKADKILILHEGSSYFYGTF
SELQNLQPDFSSKLMGCDSFDQFSAERRNSILTETLHRFSLEGDAPVSWTETKKQSFKQTGEF
GEKRKNSILNPINSIRKFSIVQKTPLQMNGIEEDSDEPLERRLSLVPDSEQGEAILPRISVIS
TGPTLQARRRQSVLNLMTHSVNQGQNIHRKTTASTRKVSLAPQANLTELDIYSRRLSQETGLE
ISEEINEEDLKECFFDDMESIPAVTTWNTYLRYITVHKSLIFVLIWCLVIFLAEVAASLVVLW
LLGNTPLQDKGNSTHSRNNSYAVIITSTSSYYVFYIYVGVADTLLAMGFFRGLPLVHTLITVS
KILHHKMLHSVLQAPMSTLNTLKAGGILNRFSKDIAILDDLLPLTIFDFIQLLLIVIGAIAVV
AVLQPYIFVATVPVIVAFIMLRAYFLQTSQQLKQLESEGRSPIFTHLVTSLKGLWTLRAFGRQ
PYFETLFHKALNLHTANWFLYLSTLRWFQMRIEMIFVIFFIAVTFISILTTGEGEGRVGIILT
LAMNIMSTLQWAVNSSIDVDSLMRSVSRVFKFIDMPTEGKPTKSTKPYKNGQLSKVMIIENSH
VKKDDIWPSGGQMTVKDLTAKYTEGGNAILENISFSISPGQRVGLLGRTGSGKSTLLSAFLRL
LNTEGEIQIDGVSWDSITLQQWRKAFGVIPQKVFIFSGTFRKNLDPYEQWSDQEIWKVADEVG
LRSVIEQFPGKLDFVLVDGGCVLSHGHKQLMCLARSVLSKAKILLLDEPSAHLDPVTYQIIRR
TLKQAFADCTVILCEHRIEAMLECQQFLVIEENKVRQYDSIQKLLNERSLFRQAISPSDRVKL
FPHRNSSKCKSKPQIAALKEETEEEVQDTRL*
(pARM2492)
SEQ ID NO: 99
MQRSPLEKASVVSKLFFSWTRPILRKGYRQRLELSDIYQIPSVDSADNLSEKLEREWDRELAS
KKNPKLINALRRCFFWRFMFYGIFLYLGEVTKAVQPLLLGRIIASYDPDNKEERSIAIYLGIG
LCLLFIVRTLLLHPAIFGLHHIGMQMRIAMFSLIYKKTLKLSSRVLDKISIGQLVSLLSNNLN
KFDEGLALAHFVWIAPLQVALLMGLIWELLQASAFCGLGFLIVLALFQAGLGRMMMKYRDQRA
GKISERLVITSEMIENIQSVKAYCWEEAMEKMIENLRQTELKLTRKAAYVRYFNSSAFFFSGF
FVVFLSVLPYALIKGIILRKIFTTISFCIVLRMAVTRQFPWAVQTWYDSLGAINKIQDFLQKQ
EYKTLEYNLTTTEVVMENVTAFWEEGFGELFEKAKQNNNNRKTSNGDDSLFFSNFSLLGTPVL
KDINFKIERGQLLAVAGSTGAGKTSLLMVIMGELEPSEGKIKHSGRISFCSQFSWIMPGTIKE
NIIFGVSYDEYRYRSVIKACQLEEDISKFAEKDNIVLGEGGITLSGGQRARISLARAVYKDAD
LYLLDSPFGYLDVLTEKEIFESCVCKLMANKTRILVTSKMEHLKKADKILILHEGSSYFYGTF
SELQNLQPDFSSKLMGCDSFDQFSAERRNSILTETLHRFSLEGDAPVSWTETKKQSFKQTGEF
GEKRKNSILNPINSIRKFSIVQKTPLQMNGIEEDSDEPLERRLSLVPDSEQGEAILPRISVIS
TGPTLQARRRQSVLNLMTHSVNQGQNIHRKTTASTRKVSLAPQANLTELDIYSRRLSQETGLE
ISEEINEEDLKECFFDDMESIPAVTTWNTYLRYITVHKSLIFVLIWCLVIFLAEVAASLVVLW
LLGNTPLQDKGNSTHSRNNSYAVIITSTSSYYVFYIYVGVADTLLAMGFFRGLPLVHTLITVS
KILHHKMLHSVLQAPMSTLNTLKAGGILNRFSKDIAILDDLLPLTIFDFIQLLLIVIGAIAVV
AVLQPYIFVATVPVIVAFIMLRAYFLQTSQQLKQLESEGRSPIFTHLVTSLKGLWTLRAFGRQ
PYFETLFHKALNLHTANWFLYLSTLRWFQMRIEMIFVIFFIAVTFISILTTGEGEGRVGIILT
LAMNIMSTLQWAVNSSIDVDSLMRSVSRVFKFIDMPTEGKPTKSTKPYKNGQLSKVMIIENSH
VKKDDIWPSGGQMTVKDLTAKYTEGGNAILENISFSISPGQRVGLLGRTGSGKSTLLSAFLRL
LNTEGEIQIDGVSWDSITLQQWRKAFGVIPQKVFIFSGTFRKNLDPYEQWSDQEIWKVADEVG
LRSVIEQFPGKLDFVLVDGGCVLSHGHKQLMCLARSVLSKAKILLLDEPSAHLDPVTYQIIRR
TLKQAFADCTVILCEHRIEAMLECQQFLVIEENKVRQYDSIQKLLNERSLFRQAISPSDRVKL
FPHRNSSKCKSKPQIAALKEETEEEVQDTRL*

### mRNA ORFs (Selection of Leads)

(1831)
SEQ ID NO: 100
AUGCAGCGCAGCCCCCUCGAGAAGGCCAGCGUGGUGAGCAAGCUGUUCUU
CAGCUGGACCCGCCCCAUCCUGCGCAAGGGCUACCGCCAGCGCCUGGAGC
UGAGCGACAUCUACCAGAUCCCCAGCGUGGACAGCGCCGACAACCUGAGC
GAGAAGCUGGAGCGCGAGUGGGACCGCGAGCUGGCCAGCAAGAAGAACCC
CAAGCUGAUCAACGCCCUGCGCCGCUGCUUCUUCUGGCGCUUCAUGUUCU
ACGGCAUCUUCCUGUACCUGGGCGAGGUGACCAAGGCCGUGCAGCCCCUG
CUGCUGGGCCGCAUCAUCGCCAGCUACGACCCCGACAACAAGGAGGAGCG
CAGCAUCGCCAUCUACCUGGGCAUCGGCCUGUGCCUGCUGUUCAUCGUGC
GCACCCUGCUGCUGCACCCCGCCAUCUUCGGCCUGCACCACAUCGGCAUG
CAGAUGCGCAUCGCCAUGUUCAGCCUGAUCUACAAGAAGACCCUGAAGCU
GAGCAGCCGCGUGCUGGACAAGAUCAGCAUCGGCCAGCUGGUGAGCCUGC
UGAGCAACAACCUGAACAAGUUCGACGAGGGCCUGGCCCUGGCCCACUUC
GUGUGGAUCGCCCCCCUGCAGGUGGCCCUGCUGAUGGGCCUGAUCUGGGA
GCUGCUGCAGGCCAGCGCCUUCUGCGGCCUGGGCUUCCUGAUCGUGCUGG
CCCUGUUCCAGGCCGGCCUGGGCCGCAUGAUGAUGAAGUACCGCGACCAG
CGCGCCGGCAAGAUCAGCGAGCGCCUGGUGAUCACCAGCGAGAUGAUCGA
GAACAUCCAGAGCGUGAAGGCCUACUGCUGGGAGGAGGCCAUGGAGAAGA
UGAUCGAGAACCUGCGCCAGACCGAGCUGAAGCUGACCCGCAAGGCCGCC
UACGUGCGCUACUUCAACAGCAGCGCCUUCUUCUUCAGCGGCUUCUUCGU
GGUGUUCCUGAGCGUGCUGCCCUACGCCCUGAUCAAGGGCAUCAUCCUGC
GCAAGAUCUUCACCACCAUCAGCUUCUGCAUCGUGCUGCGCAUGGCCGUG
ACCCGCCAGUUCCCCUGGGCCGUGCAGACCUGGUACGACAGCCUGGGCGC
CAUCAACAAGAUCCAGGACUUCCUGCAGAAGCAGGAGUACAAGACCCUGG
AGUACAACCUGACCACCACCGAGGUGGUGAUGGAGAACGUGACCGCCUUC
UGGGAGGAGGGCUUCGGCGAGCUGUUCGAGAAGGCCAAGCAGAACAACAA
CAACCGCAAGACCAGCAACGGCGACGACAGCCUGUUCUUCAGCAACUUCA
GCCUGCUGGGCACCCCCGUGCUGAAGGACAUCAACUUCAAGAUCGAGCGC
GGCCAGCUGCUGGCCGUGGCCGGCAGCACCGGCGCCGGCAAGACCAGCCU
GCUGAUGGUGAUCAUGGGCGAGCUGGAGCCCAGCGAGGGCAAGAUCAAGC
ACAGCGGCCGCAUCAGCUUCUGCAGCCAGUUCAGCUGGAUCAUGCCCGGC
ACCAUCAAGGAGAACAUCAUCUUCGGCGUGAGCUACGACGAGUACCGCUA
CCGCAGCGUGAUCAAGGCCUGCCAGCUGGAGGAGGACAUCAGCAAGUUCG
CCGAGAAGGACAACAUCGUGCUGGGCGAGGGCGGCAUCACCCUGAGCGGC
GGCCAGCGCGCCCGCAUCAGCCUGGCCCGCGCCGUGUACAAGGACGCCGA
CCUGUACCUGCUGGACAGCCCCUUCGGCUACCUGGACGUGCUGACCGAGA
AGGAGAUCUUCGAGAGCUGCGUGUGCAAGCUGAUGGCCAACAAGACCCGC
AUCCUGGUGACCAGCAAGAUGGAGCACCUGAAGAAGGCCGACAAGAUCCU
GAUCCUGCACGAGGGCAGCAGCUACUUCUACGGCACCUUCAGCGAGCUGC
AGAACCUGCAGCCCGACUUCAGCAGCAAGCUGAUGGGCUGCGACAGCUUC
GACCAGUUCAGCGCCGAGCGCCGCAACAGCAUCCUGACCGAGACCCUGCA
CCGCUUCAGCCUGGAGGGCGACGCCCCCGUGAGCUGGACCGAGACCAAGA
AGCAGAGCUUCAAGCAGACCGGCGAGUUCGGCGAGAAGCGCAAGAACAGC
AUCCUGAACCCCAUCAACAGCAUCCGCAAGUUCAGCAUCGUGCAGAAGAC
CCCCCUGCAGAUGAACGGCAUCGAGGAGGACAGCGACGAGCCCCUGGAGC
GCCGCCUGAGCCUGGUGCCCGACAGCGAGCAGGGCGAGGCCAUCCUGCCC
CGCAUCAGCGUGAUCAGCACCGGCCCCACCCUGCAGGCCCGCCGCCGCCA
GAGCGUGCUGAACCUGAUGACCCACAGCGUGAACCAGGGCCAGAACAUCC
ACCGCAAGACCACCGCCAGCACCCGCAAAGUGAGCCUGGCCCCCCAGGCC
AACCUGACCGAGCUGGACAUCUACAGCCGCCGCCUGAGCCAGGAGACCGG
CCUGGAGAUCAGCGAGGAGAUCAACGAGGAGGACCUGAAGGAGUGCUUCU
UCGACGACAUGGAGAGCAUCCCCGCCGUGACCACCUGGAACACCUACCUG
CGCUACAUCACCGUGCACAAGAGCCUGAUCUUCGUGCUGAUCUGGUGCCU
GGUGAUCUUCCUGGCCGAGGUGGCCGCCAGCCUGGUGGUGCUGUGGCUGC
UGGGCAACACCCCCCUGCAGGACAAGGGCAACAGCACCCACAGCCGCAAC
AACAGCUACGCCGUGAUCAUCACCAGCACCAGCAGCUACUACGUGUUCUA
CAUCUACGUGGGCGUGGCCGACACCCUGCUGGCCAUGGGCUUCUUCCGCG
GCCUGCCCCUGGUGCACACCCUGAUCACCGUGAGCAAGAUCCUGCACCAC
AAGAUGCUGCACAGCGUGCUGCAGGCCCCCAUGAGCACCCUGAACACCCU
GAAGGCCGGCGGCAUCCUGAACCGCUUCAGCAAGGACAUCGCCAUCCUGG
ACGACCUGCUGCCCCUGACCAUCUUCGACUUCAUCCAGCUGCUGCUGAUC
GUGAUCGGCGCCAUCGCCGUGGUGGCCGUGCUGCAGCCCUACAUCUUCGU
GGCCACCGUGCCCGUGAUCGUGGCCUUCAUCAUGCUGCGCGCCUACUUCC
UGCAGACCAGCCAGCAGCUGAAGCAGCUGGAGAGCGAGGGCCGCAGCCCC
AUCUUCACCCACCUGGUGACCAGCCUGAAGGGCCUGUGGACCCUGCGCGC
CUUCGGCCGCCAGCCCUACUUCGAGACCCUGUUCCACAAGGCCCUGAACC
UGCACACCGCCAACUGGUUCCUGUACCUGAGCACCCUGCGCUGGUUCCAG
AUGCGCAUCGAGAUGAUCUUCGUGAUCUUCUUCAUCGCCGUGACCUUCAU
CAGCAUCCUGACCACCGGCGAGGGCGAGGGCCGCGUGGGCAUCAUCCUGA
CCCUGGCCAUGAACAUCAUGAGCACCCUGCAGUGGGCCGUGAACAGCAGC
AUCGACGUGGACAGCCUGAUGCGCAGCGUGAGCCGCGUGUUCAAGUUCAU
CGACAUGCCCACCGAGGGCAAGCCCACCAAGAGCACCAAGCCCUACAAGA
ACGGCCAGCUGAGCAAGGUGAUGAUCAUCGAGAACAGCCACGUGAAGAAG
GACGACAUCUGGCCCAGCGGCGGCCAGAUGACCGUGAAGGACCUGACCGC
CAAGUACACCGAGGGCGGCAACGCCAUCCUGGAGAACAUCAGCUUCAGCA
UCAGCCCCGGCCAGCGCGUGGGCCUGCUGGGCCGCACCGGCAGCGGCAAG
AGCACCCUGCUGAGCGCCUUCCUGCGCCUGCUGAACACCGAGGGCGAGAU
CCAGAUCGACGGCGUGAGCUGGGACAGCAUCACCCUGCAGCAGUGGCGCA
AGGCCUUCGGCGUGAUCCCCCAGAAGGUGUUCAUCUUCAGCGGCACCUUC
CGCAAGAACCUGGACCCCUACGAGCAGUGGAGCGACCAGGAGAUCUGGAA
GGUGGCCGACGAGGUGGGCCUGCGCAGCGUGAUCGAGCAGUUCCCCGGCA
AGCUGGACUUCGUGCUGGUGGACGGCGGCUGCGUGCUGAGCCACGGCCAC
AAGCAGCUGAUGUGCCUGGCCCGCAGCGUGCUGAGCAAGGCCAAGAUCCU
GCUGCUGGACGAGCCCAGCGCCCACCUGGACCCCGUGACCUACCAGAUCA
UCCGCCGCACCCUGAAGCAGGCCUUCGCCGACUGCACCGUGAUCCUGUGC
GAGCACCGCAUCGAGGCCAUGCUGGAGUGCCAGCAGUUCCUGGUGAUCGA
GGAGAACAAGGUGCGCCAGUACGACAGCAUCCAGAAGCUGCUGAACGAGC
GCAGCCUGUUCCGCCAGGCCAUCAGCCCCAGCGACCGCGUGAAGCUUUUC
CCCCACCGCAACAGCAGCAAGUGCAAGAGCAAGCCCCAGAUCGCCGCCCU
GAAGGAGGAGACCGAGGAGGAGGUGCAGGACACCCGCCUGUAG
(1835)
SEQ ID NO: 101
AUGCAGAGGUCGCCUCUGGAGAAGGCCAGCGUGGUGUCCAAGCUGUUCUU
CAGCUGGACCAGACCAAUCCUGAGGAAGGGCUACAGACAGCGCCUGGAGC
UGUCAGACAUCUACCAGAUCCCUUCUGUGGACUCUGCUGACAACCUGUCU
GAGAAGCUGGAGAGAGAGUGGGACAGAGAGCUGGCCAGCAAGAAGAACCC
UAAGCUGAUCAACGCCCUGCGGAGGUGCUUCUUCUGGAGAUUCAUGUUCU
ACGGAAUCUUCCUGUACCUGGGGGAGGUGACCAAGGCCGUGCAGCCUCUG
CUGCUGGGAAGAAUCAUCGCCUCCUACGACCCCGACAACAAGGAGGAGCG
CUCUAUCGCCAUCUACCUGGGCAUCGGCCUGUGCCUGCUGUUCAUCGUGA
GGACCCUGCUGCUGCACCCAGCCAUCUUCGGCCUGCACCACAUCGGAAUG
CAGAUGAGAAUCGCCAUGUUCAGCCUGAUCUACAAGAAGACCCUGAAGCU
GUCAAGCAGGGUGCUGGACAAGAUCAGUAUCGGACAGCUGGUGAGUCUGC
UGUCCAACAACCUGAACAAGUUCGACGAGGGACUGGCCCUGGCCCACUUC
GUGUGGAUCGCUCCUCUGCAGGUGGCCCUGCUGAUGGGGCUGAUCUGGGA
GCUGCUGCAGGCCUCUGCCUUCUGCGGCCUGGGCUUCCUGAUCGUGCUGG
CCCUGUUCCAGGCCGGCCUGGGCAGAAUGAUGAUGAAGUACAGAGACCAG
AGAGCUGGCAAGAUCAGCGAGAGACUGGUGAUCACCUCAGAGAUGAUCGA
GAACAUCCAGUCUGUGAAGGCAUACUGCUGGGAGGAGGCCAUGGAGAAGA
UGAUCGAGAACCUGAGACAGACCGAGCUGAAGCUGACCCGGAAGGCCGCC
UACGUGAGAUACUUCAACAGCAGCGCCUUCUUCUUCUCAGGGUUCUUCGU
GGUGUUCCUGUCUGUGCUGCCCUACGCCCUGAUCAAGGGCAUCAUCCUGC
GGAAGAUCUUCACCACCAUCUCAUUCUGCAUCGUGCUGCGCAUGGCCGUG
ACCCGGCAGUUCCCCUGGGCCGUGCAGACCUGGUACGACUCUCUGGGAGC
CAUCAACAAGAUCCAGGACUUCCUGCAGAAGCAGGAGUACAAGACCCUGG
AGUACAACCUGACCACCACCGAGGUGGUGAUGGAGAACGUGACCGCCUUC
UGGGAGGAGGGAUUCGGCGAGCUGUUCGAGAAGGCCAAGCAGAACAACAA
CAACAGAAAGACCUCUAACGGCGACGACAGCCUGUUCUUCAGCAACUUCA
GCCUGCUGGGCACCCCUGUGCUGAAGGACAUCAACUUCAAGAUCGAGAGA
GGACAGCUGCUGGCCGUGGCCGGAUCCACCGGAGCCGGCAAGACCUCACU
GCUGAUGGUGAUCAUGGGAGAGCUGGAGCCUUCAGAGGGCAAGAUCAAGC
ACAGUGGAAGAAUCUCAUUCUGCUCUCAGUUCUCCUGGAUCAUGCCUGGC
ACCAUCAAGGAGAACAUCAUCUUCGGUGUGUCCUACGACGAGUACAGAUA
CAGAAGCGUGAUCAAGGCCUGCCAGCUGGAGGAGGACAUCUCCAAGUUCG
CAGAGAAGGACAACAUCGUGCUGGGAGAGGGUGGCAUCACCCUGAGCGGA
GGCCAGAGGGCCAGAAUCUCUCUGGCAAGAGCAGUGUACAAGGACGCUGA
CCUGUACCUGCUGGACUCUCCUUUCGGAUACCUGGACGUGCUGACCGAGA
AGGAGAUCUUCGAGAGCUGCGUGUGCAAGCUGAUGGCCAACAAGACCAGG
AUCCUGGUGACCUCUAAGAUGGAGCACCUGAAGAAGGCUGACAAGAUCCU
GAUCCUGCACGAGGGCAGCAGCUACUUCUACGGGACCUUCAGCGAGCUGC
AGAACCUGCAGCCAGACUUCAGCAGCAAGCUGAUGGGCUGCGACUCUUUC
GACCAGUUCAGCGCCGAGAGAAGAAACAGCAUCCUGACCGAGACCCUGCA
CAGGUUCAGCCUGGAGGGCGACGCCCCUGUGUCCUGGACCGAGACCAAGA
AGCAGUCUUUCAAGCAGACCGGAGAGUUCGGCGAGAAGAGGAAGAACUCU
AUCCUGAACCCAAUCAACUCUAUCAGGAAGUUCUCCAUCGUGCAGAAGAC
CCCCCUGCAGAUGAACGGCAUCGAGGAGGACUCUGACGAGCCUCUGGAGA
GAAGGCUGUCCCUGGUGCCAGACUCUGAGCAGGGCGAGGCCAUCCUGCCU
CGCAUCAGCGUGAUCAGCACCGGCCCCACCCUGCAGGCCAGGAGGAGGCA
GUCUGUGCUGAACCUGAUGACCCACAGCGUGAACCAGGGCCAGAACAUCC
ACAGGAAGACCACCGCCUCCACCAGGAAGGUGAGCCUGGCCCCUCAGGCC
AACCUGACCGAGCUGGACAUCUACAGCAGAAGGCUGUCUCAGGAGACCGG
CCUGGAGAUCAGCGAGGAGAUCAACGAGGAGGACCUGAAGGAGUGCUUCU
UCGACGACAUGGAGAGCAUCCCAGCCGUGACCACCUGGAACACCUACCUG
AGGUACAUCACCGUGCACAAGAGCCUGAUCUUCGUGCUGAUCUGGUGCCU
GGUGAUCUUCCUGGCCGAGGUGGCCGCCUCUCUGGUGGUGCUGUGGCUGC
UGGGCAACACCCCUCUGCAGGACAAGGGCAACAGCACCCACAGCAGAAAC
AACAGCUACGCCGUGAUCAUCACCAGCACCAGCAGCUACUACGUGUUCUA
CAUCUACGUGGGAGUGGCCGACACCCUGCUGGCCAUGGGCUUCUUCAGAG
GUCUGCCACUGGUGCACACCCUGAUCACCGUGAGCAAGAUCCUGCACCAC
AAGAUGCUGCACUCUGUGCUGCAGGCCCCUAUGAGCACCCUGAACACCCU
GAAGGCCGGUGGGAUCCUGAACAGAUUCUCCAAGGACAUCGCCAUCCUGG
ACGACCUGCUGCCUCUGACCAUCUUCGACUUCAUCCAGCUGCUGCUGAUC
GUGAUCGGAGCCAUCGCCGUGGUGGCCGUGCUGCAGCCCUACAUCUUCGU
GGCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCCA
AUCUUCACCCACCUGGUGACCAGCCUGAAGGGACUGUGGACCCUGAGGGC
CUUCGGCCGGCAGCCUUACUUCGAGACCCUGUUCCACAAGGCUCUGAACC
UGCACACCGCCAACUGGUUCCUGUACCUGAGCACCCUGCGCUGGUUCCAG
AUGAGAAUCGAGAUGAUCUUCGUGAUCUUCUUCAUCGCCGUGACCUUCAU
CUCCAUCCUGACCACCGGCGAGGGAGAGGGAAGAGUGGGCAUCAUCCUGA
CCCUGGCCAUGAACAUCAUGAGCACCCUGCAGUGGGCUGUGAACUCCAGC
AUCGACGUGGACAGCCUGAUGAGGUCUGUGAGCAGGGUGUUCAAGUUCAU
CGACAUGCCAACCGAGGGCAAGCCUACCAAGAGCACCAAGCCAUACAAGA
ACGGCCAGCUGAGCAAGGUGAUGAUCAUCGAGAACAGCCACGUGAAGAAG
GACGACAUCUGGCCCAGCGGCGGCCAGAUGACCGUGAAGGACCUGACCGC
CAAGUACACCGAGGGCGGCAACGCCAUCCUGGAGAACAUCUCCUUCUCAA
UCAGCCCUGGCCAGAGGGUGGGCCUGCUGGGAAGAACCGGCUCAGGCAAG
AGCACCCUGCUGAGCGCCUUCCUGAGACUGCUGAACACCGAGGGCGAGAU
CCAGAUCGACGGCGUGUCUUGGGACUCAAUCACCCUGCAGCAGUGGAGGA
AGGCCUUCGGCGUGAUCCCACAGAAGGUGUUCAUCUUCUCUGGAACCUUC
AGAAAGAACCUGGACCCCUACGAGCAGUGGAGCGACCAGGAGAUCUGGAA
GGUGGCCGACGAGGUGGGCCUGAGAUCUGUGAUCGAGCAGUUCCCUGGCA
AGCUGGACUUCGUGCUGGUGGACGGGGGCUGCGUGCUGAGCCACGGCCAC
AAGCAGCUGAUGUGCCUGGCCAGAUCUGUGCUGAGCAAGGCCAAGAUCCU
GCUGCUGGACGAGCCCAGUGCCCACCUGGACCCAGUGACCUACCAGAUCA
UCAGAAGAACCCUGAAGCAGGCCUUCGCCGACUGCACCGUGAUCCUGUGC
GAGCACAGGAUCGAGGCCAUGCUGGAGUGCCAGCAGUUCCUGGUGAUCGA
GGAGAACAAGGUGCGGCAGUACGACUCCAUCCAGAAGCUGCUGAACGAGA
GGAGCCUGUUCCGGCAGGCCAUCAGCCCCUCCGACAGGGUGAAGCUGUUC
CCCCACCGGAACAGCAGCAAGUGCAAGUCUAAGCCCCAGAUCGCCGCCCU
GAAGGAGGAGACCGAGGAGGAGGUGCAGGACACCAGGCUGUAG
(2093)
SEQ ID NO: 102
AUGCAGAGGUCGCCCCUGGAGAAGGCUAGCGUGGUGAGCAAGCUGUUCUU
CAGCUGGACCAGACCAAUCCUGAGGAAGGGCUACAGACAGCGCCUGGAGC
UGUCAGACAUCUACCAGAUCCCUAGCGUGGACAGCGCCGACAACCUGAGC
GAGAAGCUGGAGAGAGAGUGGGACAGAGAGCUGGCCAGCAAGAAGAACCC
CAAGCUGAUCAACGCCCUGCGGAGGUGCUUCUUCUGGAGAUUCAUGUUCU
ACGGAAUCUUCCUGUACCUGGGGGAGGUGACCAAGGCCGUGCAGCCCCUG
CUGCUGGGAAGAAUCAUCGCCAGCUACGACCCCGACAACAAGGAGGAGCG
CUCUAUCGCCAUCUACCUGGGCAUCGGCCUGUGCCUGCUGUUCAUCGUGA
GGACCCUGCUGCUGCACCCAGCCAUCUUCGGCCUGCACCACAUCGGAAUG
CAGAUGAGAAUCGCCAUGUUCAGCCUGAUCUACAAGAAGACCCUGAAGCU
GAGCAGCAGGGUGCUGGACAAGAUCAGUAUCGGACAGCUGGUGAGCCUGC
UGAGCAACAACCUGAACAAGUUCGACGAGGGACUGGCCCUGGCCCACUUC
GUGUGGAUCGCCCCUCUGCAGGUGGCCCUGCUGAUGGGGCUGAUCUGGGA
GCUGCUGCAGGCCAGCGCCUUCUGCGGCCUGGGCUUCCUGAUCGUGCUGG
CCCUGUUCCAGGCCGGCCUGGGCAGAAUGAUGAUGAAGUACAGAGACCAG
AGAGCUGGCAAGAUCAGCGAGAGACUGGUGAUCACCAGCGAGAUGAUCGA
GAACAUCCAGAGCGUGAAGGCAUACUGCUGGGAGGAGGCCAUGGAGAAGA
UGAUCGAGAACCUGAGACAGACCGAGCUGAAGCUGACCCGGAAGGCCGCC
UACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUGC
GGAAGAUCUUCACCACCAUCAGCUUCUGCAUCGUGCUGCGCAUGGCCGUG
ACCCGGCAGUUCCCCUGGGCCGUGCAGACCUGGUACGACUCUCUGGGAGC
CAUCAACAAGAUCCAGGACUUCCUGCAGAAGCAGGAGUACAAGACCCUGG
AGUACAACCUGACCACCACCGAGGUGGUGAUGGAGAACGUGACCGCCUUC
UGGGAGGAGGGAUUCGGCGAGCUGUUCGAGAAGGCCAAGCAGAACAACAA
CAACAGAAAGACCAGCAACGGCGACGACAGCCUGUUCUUCAGCAACUUCA
GCCUGCUGGGCACCCCCGUGCUGAAGGACAUCAACUUCAAGAUCGAGAGA
GGACAGCUGCUGGCCGUGGCCGGAAGCACCGGAGCCGGCAAGACCAGCCU
GCUGAUGGUGAUCAUGGGAGAGCUGGAGCCCAGCGAGGGCAAGAUCAAGC
ACAGCGGAAGAAUCAGCUUCUGCAGCCAGUUCUCCUGGAUCAUGCCCGGC
ACCAUCAAGGAGAACAUCAUCUUCGGUGUGAGCUACGACGAGUACAGAUA
CAGAAGCGUGAUCAAGGCCUGCCAGCUGGAGGAGGACAUCUCCAAGUUCG
CAGAGAAGGACAACAUCGUGCUGGGAGAGGGUGGCAUCACCCUGAGCGGA
GGCCAGAGGGCCAGAAUCUCUCUGGCAAGAGCAGUGUACAAGGACGCCGA
CCUGUACCUGCUGGACUCUCCCUUCGGAUACCUGGACGUGCUGACCGAGA
AGGAGAUCUUCGAGAGCUGCGUGUGCAAGCUGAUGGCCAACAAGACCAGG
AUCCUGGUGACCUCUAAGAUGGAGCACCUGAAGAAGGCCGACAAGAUCCU
GAUCCUGCACGAGGGCAGCAGCUACUUCUACGGGACCUUCAGCGAGCUGC
AGAACCUGCAGCCAGACUUCAGCAGCAAGCUGAUGGGCUGCGACAGCUUC
GACCAGUUCAGCGCCGAGAGAAGAAACAGCAUCCUGACCGAGACCCUGCA
CAGGUUCAGCCUGGAGGGCGACGCCCCCGUGAGCUGGACCGAGACCAAGA
AGCAGAGCUUCAAGCAGACCGGAGAGUUCGGCGAGAAGAGGAAGAACAGC
AUCCUGAACCCAAUCAACAGCAUCAGGAAGUUCUCCAUCGUGCAGAAGAC
CCCACUGCAGAUGAACGGCAUCGAGGAGGACAGCGACGAGCCCCUGGAGA
GAAGGCUGAGCCUGGUGCCAGACUCUGAGCAGGGCGAGGCCAUCCUGCCC
CGCAUCAGCGUGAUCAGCACCGGCCCCACCCUGCAGGCCAGGAGGAGGCA
GAGCGUGCUGAACCUGAUGACCCACAGCGUGAACCAGGGCCAGAACAUCC
ACAGGAAGACCACCGCCAGCACCAGGAAGGUGAGCCUGGCCCCACAGGCC
AACCUGACCGAGCUGGACAUCUACAGCAGAAGGCUGAGCCAGGAGACCGG
CCUGGAGAUCAGCGAGGAGAUCAACGAGGAGGACCUGAAGGAGUGCUUCU
UCGACGACAUGGAGAGCAUCCCAGCCGUGACCACCUGGAACACCUACCUG
AGGUACAUCACCGUGCACAAGAGCCUGAUCUUCGUGCUGAUCUGGUGCCU
GGUGAUCUUCCUGGCCGAGGUGGCCGCCAGCCUGGUGGUGCUGUGGCUGC
UGGGCAACACCCCACUGCAGGACAAGGGCAACAGCACCCACAGCAGAAAC
AACAGCUACGCCGUGAUCAUCACCAGCACCAGCAGCUACUACGUGUUCUA
CAUCUACGUGGGAGUGGCCGACACCCUGCUGGCCAUGGGCUUCUUCAGAG
GCCUGCCACUGGUGCACACCCUGAUCACCGUGAGCAAGAUCCUGCACCAC
AAGAUGCUGCACAGCGUGCUGCAGGCCCCCAUGAGCACCCUGAACACCCU
GAAGGCCGGUGGGAUCCUGAACAGAUUCUCCAAGGACAUCGCCAUCCUGG
ACGACCUGCUGCCCCUGACCAUCUUCGACUUCAUCCAGCUGCUGCUGAUC
GUGAUCGGAGCCAUCGCCGUGGUGGCCGUGCUGCAGCCCUACAUCUUCGU
GGCCACCGUGCCAGUGAUCGUGGCCUUCAUCAUGCUGAGAGCCUACUUCC
UGCAGACCAGCCAGCAGCUGAAGCAGCUGGAGUCUGAGGGCAGGAGUCCA
AUCUUCACCCACCUGGUGACCAGCCUGAAGGGACUGUGGACCCUGAGGGC
CUUCGGCCGGCAGCCCUACUUCGAGACCCUGUUCCACAAGGCCCUGAACC
UGCACACCGCCAACUGGUUCCUGUACCUGAGCACCCUGCGCUGGUUCCAG
AUGAGAAUCGAGAUGAUCUUCGUGAUCUUCUUCAUCGCCGUGACCUUCAU
CAGCAUCCUGACCACCGGCGAGGGAGAGGGAAGAGUGGGCAUCAUCCUGA
CCCUGGCCAUGAACAUCAUGAGCACCCUGCAGUGGGCCGUGAACAGCAGC
AUCGACGUGGACAGCCUGAUGAGGAGCGUGAGCAGGGUGUUCAAGUUCAU
CGACAUGCCAACCGAGGGCAAGCCCACCAAGAGCACCAAGCCAUACAAGA
ACGGCCAGCUGAGCAAGGUGAUGAUCAUCGAGAACAGCCACGUGAAGAAG
GACGACAUCUGGCCCAGCGGCGGCCAGAUGACCGUGAAGGACCUGACCGC
CAAGUACACCGAGGGCGGCAACGCCAUCCUGGAGAACAUCAGCUUCAGCA
UCAGCCCCGGCCAGAGGGUGGGCCUGCUGGGAAGAACCGGCAGCGGCAAG
AGCACCCUGCUGAGCGCCUUCCUGAGACUGCUGAACACCGAGGGCGAGAU
CCAGAUCGACGGCGUGAGCUGGGACUCAAUCACCCUGCAGCAGUGGAGGA
AGGCCUUCGGCGUGAUCCCACAGAAGGUGUUCAUCUUCAGCGGAACCUUC
AGAAAGAACCUGGACCCCUACGAGCAGUGGAGCGACCAGGAGAUCUGGAA
GGUGGCCGACGAGGUGGGCCUGAGAAGCGUGAUCGAGCAGUUCCCCGGCA
AGCUGGACUUCGUGCUGGUGGACGGGGGCUGCGUGCUGAGCCACGGCCAC
AAGCAGCUGAUGUGCCUGGCCAGAUCUGUGCUGAGCAAGGCCAAGAUCCU
GCUGCUGGACGAGCCCAGCGCCCACCUGGACCCAGUGACCUACCAGAUCA
UCAGAAGAACCCUGAAGCAGGCCUUCGCCGACUGCACCGUGAUCCUGUGC
GAGCACAGGAUCGAGGCCAUGCUGGAGUGCCAGCAGUUCCUGGUGAUCGA
GGAGAACAAGGUGCGGCAGUACGACAGCAUCCAGAAGCUGCUGAACGAGA
GGAGCCUGUUCCGGCAGGCCAUCAGCCCCAGCGACAGGGUGAAGCUGUUC
CCCCACCGGAACAGCAGCAAGUGCAAGAGCAAGCCCCAGAUCGCCGCCCU
GAAGGAGGAGACCGAGGAGGAGGUGCAGGACACCAGGCUGUAG
(2095)
SEQ ID NO: 103
AUGCAGAGGUCGCCCCUGGAGAAGGCUAGCGUGGUGUCCAAGCUGUUCUU
CAGCUGGACCAGACCAAUCCUGAGGAAGGGCUACAGACAGCGCCUGGAGC
UGAGCGACAUCUACCAGAUCCCCAGCGUGGACAGCGCCGACAACCUGAGC
GAGAAGCUGGAGAGAGAGUGGGACAGAGAGCUGGCCAGCAAGAAGAACCC
CAAGCUGAUCAACGCCCUGCGGAGGUGCUUCUUCUGGAGAUUCAUGUUCU
ACGGAAUCUUCCUGUACCUGGGGGAGGUGACCAAGGCCGUGCAGCCUCUG
CUGCUGGGAAGAAUCAUCGCCAGCUACGACCCCGACAACAAGGAGGAGCG
CAGCAUCGCCAUCUACCUGGGCAUCGGCCUGUGCCUGCUGUUCAUCGUGA
GGACCCUGCUGCUGCACCCAGCCAUCUUCGGCCUGCACCACAUCGGAAUG
CAGAUGAGAAUCGCCAUGUUCAGCCUGAUCUACAAGAAGACCCUGAAGCU
GUCAAGCAGGGUGCUGGACAAGAUCAGCAUCGGACAGCUGGUGAGUCUGC
UGAGCAACAACCUGAACAAGUUCGACGAGGGACUGGCCCUGGCCCACUUC
GUGUGGAUCGCUCCACUGCAGGUGGCCCUGCUGAUGGGGCUGAUCUGGGA
GCUGCUGCAGGCCAGCGCCUUCUGCGGCCUGGGCUUCCUGAUCGUGCUGG
CCCUGUUCCAGGCCGGCCUGGGCAGAAUGAUGAUGAAGUACAGAGACCAG
AGAGCUGGCAAGAUCAGCGAGAGACUGGUGAUCACCUCAGAGAUGAUCGA
GAACAUCCAGUCUGUGAAGGCAUACUGCUGGGAGGAGGCCAUGGAGAAGA
UGAUCGAGAACCUGAGACAGACCGAGCUGAAGCUGACCCGGAAGGCCGCC
UACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUGC
GGAAGAUCUUCACCACCAUCAGCUUCUGCAUCGUGCUGCGCAUGGCCGUG
ACCCGGCAGUUCCCCUGGGCCGUGCAGACCUGGUACGACUCUCUGGGAGC
CAUCAACAAGAUCCAGGACUUCCUGCAGAAGCAGGAGUACAAGACCCUGG
AGUACAACCUGACCACCACCGAGGUGGUGAUGGAGAACGUGACCGCCUUC
UGGGAGGAGGGAUUCGGCGAGCUGUUCGAGAAGGCCAAGCAGAACAACAA
CAACAGAAAGACCUCUAACGGCGACGACAGCCUGUUCUUCAGCAACUUCA
GCCUGCUGGGCACCCCCGUGCUGAAGGACAUCAACUUCAAGAUCGAGAGA
GGACAGCUGCUGGCCGUGGCCGGAUCCACCGGAGCCGGCAAGACCAGCCU
GCUGAUGGUGAUCAUGGGAGAGCUGGAGCCCUCAGAGGGCAAGAUCAAGC
ACAGUGGAAGAAUCUCAUUCUGCAGCCAGUUCUCCUGGAUCAUGCCCGGC
ACCAUCAAGGAGAACAUCAUCUUCGGCGUGAGCUACGACGAGUACAGAUA
CAGAAGCGUGAUCAAGGCCUGCCAGCUGGAGGAGGACAUCAGCAAGUUCG
CAGAGAAGGACAACAUCGUGCUGGGAGAGGGCGGCAUCACCCUGAGCGGA
GGCCAGAGGGCCAGAAUCAGCCUGGCAAGAGCAGUGUACAAGGACGCCGA
CCUGUACCUGCUGGACAGCCCCUUCGGAUACCUGGACGUGCUGACCGAGA
AGGAGAUCUUCGAGAGCUGCGUGUGCAAGCUGAUGGCCAACAAGACCAGG
AUCCUGGUGACCAGCAAGAUGGAGCACCUGAAGAAGGCCGACAAGAUCCU
GAUCCUGCACGAGGGCAGCAGCUACUUCUACGGGACCUUCAGCGAGCUGC
AGAACCUGCAGCCAGACUUCAGCAGCAAGCUGAUGGGCUGCGACAGCUUC
GACCAGUUCAGCGCCGAGAGAAGAAACAGCAUCCUGACCGAGACCCUGCA
CAGGUUCAGCCUGGAGGGCGACGCCCCCGUGAGCUGGACCGAGACCAAGA
AGCAGUCUUUCAAGCAGACCGGAGAGUUCGGCGAGAAGAGGAAGAACAGC
AUCCUGAACCCAAUCAACAGCAUCAGGAAGUUCAGCAUCGUGCAGAAGAC
CCCACUGCAGAUGAACGGCAUCGAGGAGGACAGCGACGAGCCUCUGGAGA
GAAGGCUGAGCCUGGUGCCAGACAGCGAGCAGGGCGAGGCCAUCCUGCCC
CGCAUCAGCGUGAUCAGCACCGGCCCCACCCUGCAGGCCAGGAGGAGGCA
GAGCGUGCUGAACCUGAUGACCCACAGCGUGAACCAGGGCCAGAACAUCC
ACAGGAAGACCACCGCCAGCACCAGGAAGGUGAGCCUGGCCCCUCAGGCC
AACCUGACCGAGCUGGACAUCUACAGCAGAAGGCUGAGCCAGGAGACCGG
CCUGGAGAUCAGCGAGGAGAUCAACGAGGAGGACCUGAAGGAGUGCUUCU
UCGACGACAUGGAGAGCAUCCCAGCCGUGACCACCUGGAACACCUACCUG
AGGUACAUCACCGUGCACAAGAGCCUGAUCUUCGUGCUGAUCUGGUGCCU
GGUGAUCUUCCUGGCCGAGGUGGCCGCCAGCCUGGUGGUGCUGUGGCUGC
UGGGCAACACCCCACUGCAGGACAAGGGCAACAGCACCCACAGCAGAAAC
AACAGCUACGCCGUGAUCAUCACCAGCACCAGCAGCUACUACGUGUUCUA
CAUCUACGUGGGAGUGGCCGACACCCUGCUGGCCAUGGGCUUCUUCAGAG
GCCUGCCACUGGUGCACACCCUGAUCACCGUGAGCAAGAUCCUGCACCAC
AAGAUGCUGCACAGCGUGCUGCAGGCCCCUAUGAGCACCCUGAACACCCU
GAAGGCCGGCGGGAUCCUGAACAGAUUCAGCAAGGACAUCGCCAUCCUGG
ACGACCUGCUGCCCCUGACCAUCUUCGACUUCAUCCAGCUGCUGCUGAUC
GUGAUCGGAGCCAUCGCCGUGGUGGCCGUGCUGCAGCCCUACAUCUUCGU
GGCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGUCCA
AUCUUCACCCACCUGGUGACCAGCCUGAAGGGACUGUGGACCCUGAGGGC
CUUCGGCCGGCAGCCUUACUUCGAGACCCUGUUCCACAAGGCCCUGAACC
UGCACACCGCCAACUGGUUCCUGUACCUGAGCACCCUGCGCUGGUUCCAG
AUGAGAAUCGAGAUGAUCUUCGUGAUCUUCUUCAUCGCCGUGACCUUCAU
CUCCAUCCUGACCACCGGCGAGGGAGAGGGAAGAGUGGGCAUCAUCCUGA
CCCUGGCCAUGAACAUCAUGAGCACCCUGCAGUGGGCCGUGAACAGCAGC
AUCGACGUGGACAGCCUGAUGAGGAGCGUGAGCAGGGUGUUCAAGUUCAU
CGACAUGCCAACCGAGGGCAAGCCCACCAAGAGCACCAAGCCAUACAAGA
ACGGCCAGCUGAGCAAGGUGAUGAUCAUCGAGAACAGCCACGUGAAGAAG
GACGACAUCUGGCCCAGCGGCGGCCAGAUGACCGUGAAGGACCUGACCGC
CAAGUACACCGAGGGCGGCAACGCCAUCCUGGAGAACAUCAGCUUCAGCA
UCAGCCCCGGCCAGAGGGUGGGCCUGCUGGGAAGAACCGGCUCAGGCAAG
AGCACCCUGCUGAGCGCCUUCCUGAGACUGCUGAACACCGAGGGCGAGAU
CCAGAUCGACGGCGUGAGCUGGGACAGCAUCACCCUGCAGCAGUGGAGGA
AGGCCUUCGGCGUGAUCCCACAGAAGGUGUUCAUCUUCAGCGGAACCUUC
AGAAAGAACCUGGACCCCUACGAGCAGUGGAGCGACCAGGAGAUCUGGAA
GGUGGCCGACGAGGUGGGCCUGAGAAGCGUGAUCGAGCAGUUCCCCGGCA
AGCUGGACUUCGUGCUGGUGGACGGGGGCUGCGUGCUGAGCCACGGCCAC
AAGCAGCUGAUGUGCCUGGCCAGAAGCGUGCUGAGCAAGGCCAAGAUCCU
GCUGCUGGACGAGCCCAGCGCCCACCUGGACCCAGUGACCUACCAGAUCA
UCAGAAGAACCCUGAAGCAGGCCUUCGCCGACUGCACCGUGAUCCUGUGC
GAGCACAGGAUCGAGGCCAUGCUGGAGUGCCAGCAGUUCCUGGUGAUCGA
GGAGAACAAGGUGCGGCAGUACGACUCCAUCCAGAAGCUGCUGAACGAGA
GGAGCCUGUUCCGGCAGGCCAUCAGCCCCUCCGACAGGGUGAAGCUGUUC
CCCCACCGGAACAGCAGCAAGUGCAAGAGCAAGCCCCAGAUCGCCGCCCU
GAAGGAGGAGACCGAGGAGGAGGUGCAGGACACCAGGCUGUAG
(2096)
SEQ ID NO: 104
AUGCAGAGGUCGCCCCUGGAGAAGGCUAGCGUGGUGAGCAAGCUGUUCUU
CAGCUGGACCAGACCAAUCCUGAGGAAGGGCUACAGACAGCGCCUGGAGC
UGAGCGACAUCUACCAGAUCCCUAGCGUGGACAGCGCCGACAACCUGAGC
GAGAAGCUGGAGAGAGAGUGGGACAGAGAGCUGGCCAGCAAGAAGAACCC
CAAGCUGAUCAACGCCCUGCGGAGGUGCUUCUUCUGGAGAUUCAUGUUCU
ACGGAAUCUUCCUGUACCUGGGGGAGGUGACCAAGGCCGUGCAGCCUCUG
CUGCUGGGAAGAAUCAUCGCCAGCUACGACCCCGACAACAAGGAGGAGCG
CAGCAUCGCCAUCUACCUGGGCAUCGGCCUGUGCCUGCUGUUCAUCGUGA
GGACCCUGCUGCUGCACCCAGCCAUCUUCGGCCUGCACCACAUCGGAAUG
CAGAUGAGAAUCGCCAUGUUCAGCCUGAUCUACAAGAAGACCCUGAAGCU
GAGCAGCAGGGUGCUGGACAAGAUCAGUAUCGGACAGCUGGUGAGCCUGC
UGAGCAACAACCUGAACAAGUUCGACGAGGGACUGGCCCUGGCCCACUUC
GUGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUGG
CCCUGUUCCAGGCCGGCCUGGGCAGAAUGAUGAUGAAGUACAGAGACCAG
AGAGCCGGCAAGAUCAGCGAGAGACUGGUGAUCACCUCAGAGAUGAUCGA
GAACAUCCAGAGCGUGAAGGCAUACUGCUGGGAGGAGGCCAUGGAGAAGA
UGAUCGAGAACCUGAGACAGACCGAGCUGAAGCUGACCCGGAAGGCCGCC
UACGUGAGAUACUUCAACAGCAGCGCCUUCUUCUUCAGCGGGUUCUUCGU
GGUGUUCCUGUCUGUGCUGCCCUACGCCCUGAUCAAGGGCAUCAUCCUGC
GGAAGAUCUUCACCACCAUCUCAUUCUGCAUCGUGCUGCGCAUGGCCGUG
ACCCGGCAGUUCCCCUGGGCCGUGCAGACCUGGUACGACAGCCUGGGAGC
CAUCAACAAGAUCCAGGACUUCCUGCAGAAGCAGGAGUACAAGACCCUGG
AGUACAACCUGACCACCACCGAGGUGGUGAUGGAGAACGUGACCGCCUUC
UGGGAGGAGGGAUUCGGCGAGCUGUUCGAGAAGGCCAAGCAGAACAACAA
CAACAGAAAGACCUCUAACGGCGACGACAGCCUGUUCUUCAGCAACUUCA
GCCUGCUGGGCACCCCUGUGCUGAAGGACAUCAACUUCAAGAUCGAGAGA
GGACAGCUGCUGGCCGUGGCCGGAAGCACCGGAGCCGGCAAGACCAGCCU
GCUGAUGGUGAUCAUGGGAGAGCUGGAGCCCUCAGAGGGCAAGAUCAAGC
ACAGCGGAAGAAUCAGCUUCUGCAGCCAGUUCAGCUGGAUCAUGCCCGGC
ACCAUCAAGGAGAACAUCAUCUUCGGUGUGAGCUACGACGAGUACAGAUA
CAGAAGCGUGAUCAAGGCCUGCCAGCUGGAGGAGGACAUCAGCAAGUUCG
CAGAGAAGGACAACAUCGUGCUGGGAGAGGGCGGCAUCACCCUGAGCGGA
GGCCAGAGGGCCAGAAUCUCUCUGGCAAGAGCAGUGUACAAGGACGCCGA
CCUGUACCUGCUGGACAGCCCCUUCGGAUACCUGGACGUGCUGACCGAGA
AGGAGAUCUUCGAGAGCUGCGUGUGCAAGCUGAUGGCCAACAAGACCAGG
AUCCUGGUGACCAGCAAGAUGGAGCACCUGAAGAAGGCCGACAAGAUCCU
GAUCCUGCACGAGGGCAGCAGCUACUUCUACGGGACCUUCAGCGAGCUGC
AGAACCUGCAGCCAGACUUCAGCAGCAAGCUGAUGGGCUGCGACAGCUUC
GACCAGUUCAGCGCCGAGAGAAGAAACAGCAUCCUGACCGAGACCCUGCA
CAGGUUCAGCCUGGAGGGCGACGCCCCCGUGUCCUGGACCGAGACCAAGA
AGCAGAGCUUCAAGCAGACCGGAGAGUUCGGCGAGAAGAGGAAGAACAGC
AUCCUGAACCCAAUCAACUCUAUCAGGAAGUUCAGCAUCGUGCAGAAGAC
CCCACUGCAGAUGAACGGCAUCGAGGAGGACAGCGACGAGCCCCUGGAGA
GAAGGCUGUCCCUGGUGCCAGACAGCGAGCAGGGCGAGGCCAUCCUGCCC
CGCAUCAGCGUGAUCAGCACCGGCCCCACCCUGCAGGCCAGGAGGAGGCA
GAGCGUGCUGAACCUGAUGACCCACAGCGUGAACCAGGGCCAGAACAUCC
ACAGGAAGACCACCGCCAGCACCAGGAAGGUGAGCCUGGCCCCACAGGCC
AACCUGACCGAGCUGGACAUCUACAGCAGAAGGCUGAGCCAGGAGACCGG
CCUGGAGAUCAGCGAGGAGAUCAACGAGGAGGACCUGAAGGAGUGCUUCU
UCGACGACAUGGAGAGCAUCCCAGCCGUGACCACCUGGAACACCUACCUG
AGGUACAUCACCGUGCACAAGAGCCUGAUCUUCGUGCUGAUCUGGUGCCU
GGUGAUCUUCCUGGCCGAGGUGGCCGCCUCUCUGGUGGUGCUGUGGCUGC
UGGGCAACACCCCACUGCAGGACAAGGGCAACAGCACCCACAGCAGAAAC
AACAGCUACGCCGUGAUCAUCACCAGCACCAGCAGCUACUACGUGUUCUA
CAUCUACGUGGGAGUGGCCGACACCCUGCUGGCCAUGGGCUUCUUCAGAG
GCCUGCCACUGGUGCACACCCUGAUCACCGUGAGCAAGAUCCUGCACCAC
AAGAUGCUGCACAGCGUGCUGCAGGCCCCUAUGAGCACCCUGAACACCCU
GAAGGCCGGCGGGAUCCUGAACAGAUUCAGCAAGGACAUCGCCAUCCUGG
ACGACCUGCUGCCCCUGACCAUCUUCGACUUCAUCCAGCUGCUGCUGAUC
GUGAUCGGAGCCAUCGCCGUGGUGGCCGUGCUGCAGCCCUACAUCUUCGU
GGCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCCA
AUCUUCACCCACCUGGUGACCAGCCUGAAGGGACUGUGGACCCUGAGGGC
CUUCGGCCGGCAGCCCUACUUCGAGACCCUGUUCCACAAGGCUCUGAACC
UGCACACCGCCAACUGGUUCCUGUACCUGAGCACCCUGCGCUGGUUCCAG
AUGAGAAUCGAGAUGAUCUUCGUGAUCUUCUUCAUCGCCGUGACCUUCAU
CUCCAUCCUGACCACCGGCGAGGGAGAGGGAAGAGUGGGCAUCAUCCUGA
CCCUGGCCAUGAACAUCAUGAGCACCCUGCAGUGGGCUGUGAACAGCAGC
AUCGACGUGGACAGCCUGAUGAGGAGCGUGAGCAGGGUGUUCAAGUUCAU
CGACAUGCCAACCGAGGGCAAGCCUACCAAGAGCACCAAGCCAUACAAGA
ACGGCCAGCUGAGCAAGGUGAUGAUCAUCGAGAACAGCCACGUGAAGAAG
GACGACAUCUGGCCCAGCGGCGGCCAGAUGACCGUGAAGGACCUGACCGC
CAAGUACACCGAGGGCGGCAACGCCAUCCUGGAGAACAUCAGCUUCUCAA
UCAGCCCUGGCCAGAGGGUGGGCCUGCUGGGAAGAACCGGCAGCGGCAAG
AGCACCCUGCUGAGCGCCUUCCUGAGACUGCUGAACACCGAGGGCGAGAU
CCAGAUCGACGGCGUGAGCUGGGACAGCAUCACCCUGCAGCAGUGGAGGA
AGGCCUUCGGCGUGAUCCCACAGAAGGUGUUCAUCUUCAGCGGAACCUUC
AGAAAGAACCUGGACCCCUACGAGCAGUGGAGCGACCAGGAGAUCUGGAA
GGUGGCCGACGAGGUGGGCCUGAGAAGCGUGAUCGAGCAGUUCCCUGGCA
AGCUGGACUUCGUGCUGGUGGACGGGGGCUGCGUGCUGAGCCACGGCCAC
AAGCAGCUGAUGUGCCUGGCCAGAAGCGUGCUGAGCAAGGCCAAGAUCCU
GCUGCUGGACGAGCCCAGCGCCCACCUGGACCCAGUGACCUACCAGAUCA
UCAGAAGAACCCUGAAGCAGGCCUUCGCCGACUGCACCGUGAUCCUGUGC
GAGCACAGGAUCGAGGCCAUGCUGGAGUGCCAGCAGUUCCUGGUGAUCGA
GGAGAACAAGGUGCGGCAGUACGACAGCAUCCAGAAGCUGCUGAACGAGA
GGAGCCUGUUCCGGCAGGCCAUCAGCCCCAGCGACAGGGUGAAGCUGUUC
CCCCACCGGAACAGCAGCAAGUGCAAGUCUAAGCCCCAGAUCGCCGCCCU
GAAGGAGGAGACCGAGGAGGAGGUGCAGGACACCAGGCUGUAG
(2099)
SEQ ID NO: 105
AUGCAGAGGAGCCCCCUGGAGAAGGCUAGCGUGGUGAGCAAGCUGUUCUU
CAGCUGGACCAGACCAAUCCUGAGGAAGGGCUACAGACAGCGCCUGGAGC
UGAGCGACAUCUACCAGAUCCCCAGCGUGGACAGCGCCGACAACCUGAGC
GAGAAGCUGGAGAGAGAGUGGGACAGAGAGCUGGCCAGCAAGAAGAACCC
CAAGCUGAUCAACGCCCUGCGGAGGUGCUUCUUCUGGAGAUUCAUGUUCU
ACGGAAUCUUCCUGUACCUGGGGGAGGUGACCAAGGCCGUGCAGCCCCUG
CUGCUGGGAAGAAUCAUCGCCAGCUACGACCCCGACAACAAGGAGGAGCG
CAGCAUCGCCAUCUACCUGGGCAUCGGCCUGUGCCUGCUGUUCAUCGUGA
GGACCCUGCUGCUGCACCCAGCCAUCUUCGGCCUGCACCACAUCGGAAUG
CAGAUGAGAAUCGCCAUGUUCAGCCUGAUCUACAAGAAGACCCUGAAGCU
GAGCAGCAGGGUGCUGGACAAGAUCAGCAUCGGACAGCUGGUGAGCCUGC
UGAGCAACAACCUGAACAAGUUCGACGAGGGACUGGCCCUGGCCCACUUC
GUGUGGAUCGCCCCACUGCAGGUGGCCCUGCUGAUGGGGCUGAUCUGGGA
GCUGCUGCAGGCCAGCGCCUUCUGCGGCCUGGGCUUCCUGAUCGUGCUGG
CCCUGUUCCAGGCCGGCCUGGGCAGAAUGAUGAUGAAGUACAGAGACCAG
AGAGCCGGCAAGAUCAGCGAGAGACUGGUGAUCACCAGCGAGAUGAUCGA
GAACAUCCAGAGCGUGAAGGCAUACUGCUGGGAGGAGGCCAUGGAGAAGA
UGAUCGAGAACCUGAGACAGACCGAGCUGAAGCUGACCCGGAAGGCCGCC
UACGUGAGAUACUUCAACAGCAGCGCCUUCUUCUUCAGCGGGUUCUUCGU
GGUGUUCCUGAGCGUGCUGCCCUACGCCCUGAUCAAGGGCAUCAUCCUGC
GGAAGAUCUUCACCACCAUCAGCUUCUGCAUCGUGCUGCGCAUGGCCGUG
ACCCGGCAGUUCCCCUGGGCCGUGCAGACCUGGUACGACAGCCUGGGAGC
CAUCAACAAGAUCCAGGACUUCCUGCAGAAGCAGGAGUACAAGACCCUGG
AGUACAACCUGACCACCACCGAGGUGGUGAUGGAGAACGUGACCGCCUUC
UGGGAGGAGGGAUUCGGCGAGCUGUUCGAGAAGGCCAAGCAGAACAACAA
CAACAGAAAGACCAGCAACGGCGACGACAGCCUGUUCUUCAGCAACUUCA
GCCUGCUGGGCACCCCCGUGCUGAAGGACAUCAACUUCAAGAUCGAGAGA
GGACAGCUGCUGGCCGUGGCCGGAAGCACCGGAGCCGGCAAGACCAGCCU
GCUGAUGGUGAUCAUGGGAGAGCUGGAGCCCAGCGAGGGCAAGAUCAAGC
ACAGCGGAAGAAUCAGCUUCUGCAGCCAGUUCAGCUGGAUCAUGCCCGGC
ACCAUCAAGGAGAACAUCAUCUUCGGCGUGAGCUACGACGAGUACAGAUA
CAGAAGCGUGAUCAAGGCCUGCCAGCUGGAGGAGGACAUCAGCAAGUUCG
CAGAGAAGGACAACAUCGUGCUGGGAGAGGGCGGCAUCACCCUGAGCGGA
GGCCAGAGGGCCAGAAUCAGCCUGGCAAGAGCAGUGUACAAGGACGCCGA
CCUGUACCUGCUGGACAGCCCCUUCGGAUACCUGGACGUGCUGACCGAGA
AGGAGAUCUUCGAGAGCUGCGUGUGCAAGCUGAUGGCCAACAAGACCAGG
AUCCUGGUGACCAGCAAGAUGGAGCACCUGAAGAAGGCCGACAAGAUCCU
GAUCCUGCACGAGGGCAGCAGCUACUUCUACGGGACCUUCAGCGAGCUGC
AGAACCUGCAGCCAGACUUCAGCAGCAAGCUGAUGGGCUGCGACAGCUUC
GACCAGUUCAGCGCCGAGAGAAGAAACAGCAUCCUGACCGAGACCCUGCA
CAGGUUCAGCCUGGAGGGCGACGCCCCCGUGAGCUGGACCGAGACCAAGA
AGCAGAGCUUCAAGCAGACCGGAGAGUUCGGCGAGAAGAGGAAGAACAGC
AUCCUGAACCCAAUCAACAGCAUCAGGAAGUUCAGCAUCGUGCAGAAGAC
CCCACUGCAGAUGAACGGCAUCGAGGAGGACAGCGACGAGCCCCUGGAGA
GAAGGCUGAGCCUGGUGCCAGACAGCGAGCAGGGCGAGGCCAUCCUGCCC
CGCAUCAGCGUGAUCAGCACCGGCCCCACCCUGCAGGCCAGGAGGAGGCA
GAGCGUGCUGAACCUGAUGACCCACAGCGUGAACCAGGGCCAGAACAUCC
ACAGGAAGACCACCGCCAGCACCAGGAAGGUGAGCCUGGCCCCACAGGCC
AACCUGACCGAGCUGGACAUCUACAGCAGAAGGCUGAGCCAGGAGACCGG
CCUGGAGAUCAGCGAGGAGAUCAACGAGGAGGACCUGAAGGAGUGCUUCU
UCGACGACAUGGAGAGCAUCCCAGCCGUGACCACCUGGAACACCUACCUG
AGGUACAUCACCGUGCACAAGAGCCUGAUCUUCGUGCUGAUCUGGUGCCU
GGUGAUCUUCCUGGCCGAGGUGGCCGCCAGCCUGGUGGUGCUGUGGCUGC
UGGGCAACACCCCACUGCAGGACAAGGGCAACAGCACCCACAGCAGAAAC
AACAGCUACGCCGUGAUCAUCACCAGCACCAGCAGCUACUACGUGUUCUA
CAUCUACGUGGGAGUGGCCGACACCCUGCUGGCCAUGGGCUUCUUCAGAG
GCCUGCCACUGGUGCACACCCUGAUCACCGUGAGCAAGAUCCUGCACCAC
AAGAUGCUGCACAGCGUGCUGCAGGCCCCCAUGAGCACCCUGAACACCCU
GAAGGCCGGCGGGAUCCUGAACAGAUUCAGCAAGGACAUCGCCAUCCUGG
ACGACCUGCUGCCCCUGACCAUCUUCGACUUCAUCCAGCUGCUGCUGAUC
GUGAUCGGAGCCAUCGCCGUGGUGGCCGUGCUGCAGCCCUACAUCUUCGU
GGCCACCGUGCCAGUGAUCGUGGCCUUCAUCAUGCUGAGAGCCUACUUCC
UGCAGACCAGCCAGCAGCUGAAGCAGCUGGAGAGCGAGGGCAGGAGCCCA
AUCUUCACCCACCUGGUGACCAGCCUGAAGGGACUGUGGACCCUGAGGGC
CUUCGGCCGGCAGCCCUACUUCGAGACCCUGUUCCACAAGGCCCUGAACC
UGCACACCGCCAACUGGUUCCUGUACCUGAGCACCCUGCGCUGGUUCCAG
AUGAGAAUCGAGAUGAUCUUCGUGAUCUUCUUCAUCGCCGUGACCUUCAU
CUCCAUCCUGACCACCGGCGAGGGAGAGGGAAGAGUGGGCAUCAUCCUGA
CCCUGGCCAUGAACAUCAUGAGCACCCUGCAGUGGGCUGUGAACUCCAGC
AUCGACGUGGACAGCCUGAUGAGGUCUGUGAGCAGGGUGUUCAAGUUCAU
CGACAUGCCAACCGAGGGCAAGCCUACCAAGAGCACCAAGCCAUACAAGA
ACGGCCAGCUGAGCAAGGUGAUGAUCAUCGAGAACAGCCACGUGAAGAAG
GACGACAUCUGGCCCAGCGGCGGCCAGAUGACCGUGAAGGACCUGACCGC
CAAGUACACCGAGGGCGGCAACGCCAUCCUGGAGAACAUCUCCUUCUCAA
UCAGCCCUGGCCAGAGGGUGGGCCUGCUGGGAAGAACCGGCAGCGGCAAG
AGCACCCUGCUGAGCGCCUUCCUGAGACUGCUGAACACCGAGGGCGAGAU
CCAGAUCGACGGCGUGUCUUGGGACUCAAUCACCCUGCAGCAGUGGAGGA
AGGCCUUCGGCGUGAUCCCACAGAAGGUGUUCAUCUUCUCUGGAACCUUC
AGAAAGAACCUGGACCCCUACGAGCAGUGGAGCGACCAGGAGAUCUGGAA
GGUGGCCGACGAGGUGGGCCUGAGAUCUGUGAUCGAGCAGUUCCCUGGCA
AGCUGGACUUCGUGCUGGUGGACGGGGGCUGCGUGCUGAGCCACGGCCAC
AAGCAGCUGAUGUGCCUGGCCAGAUCUGUGCUGAGCAAGGCCAAGAUCCU
GCUGCUGGACGAGCCCAGUGCCCACCUGGACCCAGUGACCUACCAGAUCA
UCAGAAGAACCCUGAAGCAGGCCUUCGCCGACUGCACCGUGAUCCUGUGC
GAGCACAGGAUCGAGGCCAUGCUGGAGUGCCAGCAGUUCCUGGUGAUCGA
GGAGAACAAGGUGCGGCAGUACGACUCCAUCCAGAAGCUGCUGAACGAGA
GGAGCCUGUUCCGGCAGGCCAUCAGCCCCUCCGACAGGGUGAAGCUGUUC
CCCCACCGGAACAGCAGCAAGUGCAAGUCUAAGCCCCAGAUCGCCGCCCU
GAAGGAGGAGACCGAGGAGGAGGUGCAGGACACCAGGCUGUAG

### UTRs mRNAs

TABLE 39
(5′UTR sequences)

| Name | Sequence | SEQ ID NO: |
|---|---|---|
| TEV | UCAACACAACAUAUACAAAACAAACG | SEQ ID NO: 106 |
| | AAUCUCAAGCAAUCAAGCAUUCUACU | |
| | UCUAUUGCAGCAAUUUAAAUCAUUUC | |
| | UUUUAAAGCAAAAGCAAUUUUCUGAA | |
| | AAUUUUCACCAUUUACGAACGAUAG | |
| AT1G58420 | AUUAUUACAUCAAAACAAAAAGCCGC | SEQ ID NO: 107 |
| | CA | |
| HUMAN IL-6 | AAUAUUAGAGUCUCAACCCCCAAUAA | SEQ ID NO: 108 |
| | AUAUAGGACUGGAGAUGUCUGAGGCU | |
| | CAUUCUGCCCUCGAGCCCACCGGGAAC | |
| | GAAAGAGAAGCUCUAUCUCCCCUCCA | |
| | GGAGCCCAGCU | |
| MOUSE ALBUMIN | UGCACACAGAUCACCUUUCCUAUCAA | SEQ ID NO: 109 |
| | CCCCACUAGCCUCUGGCAAA | |
| MOUSE BETA-GLOBIN | ACALrUUGCUUCUGACACAACUGUGUU | SEQ ID NO: 110 |
| | CACUAGCAACCUCAAACAGACACC | |
| HUMAN CFTR | AAUUGGAAGCAAAUGACAUCACAGCA | SEQ ID NO: 111 |
| | GGUCAGAGAAAAAGGGUUGAGCGGCA | |
| | GGCACCCAGAGUAGUAGGUCUUUGGC | |
| | AUUAGGAGCUUGAGCCCAGACGGCCC | |
| | UAGCAGGGACCCCAGCGCCCGAGAGA | |
| | CC | |
| RESPIRATORY | ACGCGAAAAAAUGCGUACAACAAACU | SEQ ID NO: 112 |
| SYNCYTIAL VIRUS | UGCGUAAACCAAAAAAAU | |
| (RSV) | | |
| HUMAN SURFACTANT- | GACUUGGAGGCAGAGACCCAAGCAGC | SEQ ID NO: 113 |
| A1 | UGGAGGCUCUGUGUGUGGCCUGGAGA | |
| | CCCCACAACCUCCAGCCGGAGGCCUGA | |
| | AGC | |
| HUMAN SURFACTANT | AACUUGGAGGCAGAGACCCAAGCAGC | SEQ ID NO: 114 |
| PROTEIN A2 | UGGAGGCUCUGUGUGUGGGUCGCUGA | |
| | UUUCUUGGAGCCUGAAAAGAAGGAGC | |
| | AGCGACUGGACCCAGAGCC | |
| HUMAN NAPSIN A | GGGAAAGAAAAUGAGGCCCCAGGACA | SEQ ID NO: 115 |
| ASPARTIC PEPTIDASE | CCUGGGUUCACACCCAGGUCCCCAGCG | |
| (NAPSA) | | |
| HUMAN | AGCGCAGGGCGGUAACUCUGGGCGGG | SEQ ID NO: 116 |
| CARBOXYLESTERASE 1 | GCUGGGCUCCAGGGCUGGACAGCACA | |
| (CES1) | GUCCCUCUGAACUGCACAGAGACCUC | |
| | GCAGGCCCCGAGAACUGUCGCCCUUCC | |
| | ACG | |
| HUMAN CEACAM6 | GACCCUGGGAAAUGCUUCUAUCCCUG | SEQ ID NO: 117 |
| | AGAGGAGGCUCAGCACAGAAGGAGGA | |
| | AGGACAGCAGGGCCAACAGUCACAGC | |
| | AGCCCUGACCAGAGCAUUCCUGGAGC | |
| | UCAAGCUCCUCUACAAAGAGGUGGAC | |
| | AGAGAAGACAGCAGAGACC | |
| HUMAN GROWTH | AGGAUCCCAAGGCCCAACUCCCCGAAC | SEQ ID NO: 118 |
| HORMONE | CACUCAGGGUCCUGUGGACAGCUCAC | |
| | CUAGCUGCA | |
| INFLUENZA A/HA | AGCAAAAGCAGGGGAUAAUUCUAUUA | SEQ ID NO: 119 |
| | ACC | |
| INFLUENZA A/NS1 | AGCAAAAGCAGGGUGACAAAGACAUA | SEQ ID NO: 120 |
| INFLUENZA A/M1 | AGCAAAAGCAGGUAGAUAUUGAAAG | SEQ ID NO: 121 |
| INFLUENZA A/NP | AGCAAAAGCAGGGUUAAUAAUCACUC | SEQ ID NO: 122 |
| | ACCGAGUGACAUCAAAAUC | |
| HHV | CAGAUCGCCUGGAGACGCCAUCCACGC | SEQ ID NO: 123 |
| | UGUUUUGACCUCCAUAGAAGACACCG | |
| | GGACCGAUCCAGCCUCCGCGGCCGGGA | |
| | ACGGUGCAUUGGAACGCGGAUUCCCC | |
| | GUCAGAUCGCCUGGAGACGCCAUCCA | |
| | CGCUGUUUUGACCUCCAUAGAAGACA | |
| | CCGGGACCGAUCCAGCCUCCGCGGCCG | |
| | GGAACGGUGCAUUGGAACGCGGAUUC | |
| | CCCGUGCCAAGAGUGACUCACCGUCCU | |
| | UGACACG | |
| HUMAN HSP27 (HSPB1) | GCAUGGGGAGGGGCGGCCCUCAAACG | SEQ ID NO: 124 |
| | GGUCAUUGCCAUUAAUAGAGACCUCA | |
| | AACACCGCCUGCUAAAAAUACCCGAC | |
| | UGGAGGAGCAUAAAAGCGCAGCCGAG | |
| | CCCAGCGCCCCGCACUUUUCUGAGCAG | |
| | ACGUCCAGAGCAGAGUCAGCCAGC | |
| HUMAN HSP70 | CCUUCUGGAAGGUUCUAAGAUAGGGU | SEQ ID NO: 125 |
| | AUAAGAGGCAGGGUGGCGGGCGGAAA | |
| | CCGGUCUCAUUGAACUCGCCUGCAGC | |
| | UCUUGGGUUUUUUGUGGCUUCCUUCG | |
| | UUAUUGGAGCCAGGCCUACACCCCAG | |
| | CAACC | |

TABLE 40
(3′UTR sequences)

| Name | Sequence | SEQ ID NO: |
|---|---|---|
| XBG | CUAGUGACUGACUAGGAUCUGGUUACCACUAA | SEQ ID NO: 126 |
| | ACCAGCCUCAAGAACACCCGAAUGGAGUCUCU | |
| | AAGCUACAUAAUACCAACUUACACUUACAAAA | |
| | UGUUGUCCCCCAAAAUGUAGCCAUUCGUAUCU | |
| | GCUCCUAAUAAAAAGAAAGUUUCUUCACAU | |
| MOUSE | ACACAUCACAACCACAACCUUCUCAGGCUACCC | SEQ ID NO: 127 |
| ALBUMIN | UGAGAAAAAAAGACAUGAAGACUCAGGACUCA | |
| | UCUUUUCUGUUGGUGUAAAAUCAACACCCUAA | |
| | GGAACACAAAUUUCUUUAAACAUUUGACUUCU | |
| | UGUCUCUGUGCUGCAAUUAAUAAAAAAUGGAA | |
| | AGAAUCUAC | |
| ALANINE | GCACCCCAGCUGGGGCCAGGCUGGGUCGCCCU | SEQ ID NO: 128 |
| AMINOTRANSF | GGACUGUGUGCUCAGGAGCCCUGGGAGGCUCU | |
| ERASE 1 | GGAGCCCACUGUACUUGCUCUUGAUGCCUGGC | |
| | GGGGUGGGGUGGGGGGGGUGCUGGGCCCCUGC | |
| | CUCUCUGCAGGUCCCUAAUAAAGCUGUGUGGC | |
| | AGUCUGACUCC | |
| HUMAN | UGGCAUCCCUGUGACCCCUCCCCAGUGCCUCUC | SEQ ID NO: 129 |
| GROWTH | CUGGCCCUGGAAGUUGCCACUCCAGUGCCCAC | |
| FACTOR | CAGCCUUGUCCUAAUAAAAUUAAGUUGCAUCA | |
| | UUUUGUCUG | |
| HUMAN | ACGCCGAAGCCUGCAGCCAUGCGACCCCACGCC | SEQ ID NO: 130 |
| APOLIPOPROT | ACCCCGUGCCUCCUGCCUCCGCGCAGCCUGCAG | |
| EINE | CGGGAGACCCUGUCCCCGCCCCAGCCGUCCUCC | |
| | UGGGGUGGACCCUAGUUUAAUAAAGAUUCACC | |
| | AAGUUUCACGCA | |
| MALAT | GAUUCGUCAGUAGGGUUGUAAAGGUUUUUCUU | SEQ ID NO: 131 |
| | UUCCUGAGAAAACAACCUUUUGUUUUCUCAGG | |
| | UUUUGCUUUUUGGCCUUUCCCUAGCUUUAAAA | |
| | AAAAAAAAGCAAAAGACGCUGGUGGCUGGCAC | |
| | UCCUGGUUUCCAGGACGGGGUUCAAGUCCCUG | |
| HUMAN CFTR | AGAGCAGCAUAAAUGUUGACAUGGGACAUUUG | SEQ ID NO: 132 |
| | CUCAUGGAAUUGGAGCUCGUGGGACAGUCACC | |
| | UCAUGGAAUUGGAGCUCGUGGAACAGUUACCU | |
| | CUGCCUCAGAAAACAAGGAUGAAUUAAGUUUU | |
| | UUUUUAAAAAAGAAACAUUUGGUAAGGGGAA | |
| | UUGAGGACACUGAUAUGGGUCUUGAUAAAUGG | |
| | CUUCCUGGCAAUAGUCAAAUUGUGUGAAAGGU | |
| | ACUUCAAAUCCUUGAAGAUUUACCACUUGUGU | |
| | UUUGCAAGCCAGAUUUUCCUGAAAACCCUUGC | |
| | CAUGUGCUAGUAAUUGGAAAGGCAGCUCUAAA | |
| | UGUCAAUCAGCCUAGUUGAUCAGCUUAUUGUC | |
| | UAGUGAAACUCGUUAAUUUGUAGUGUUGGAGA | |
| | AGAACUGAAAUCAUACUUCUUAGGGUUAUGAU | |
| | UAAGUAAUGAUAACUGGAAACUUCAGCGGUUU | |
| | AUAUAAGCUUGUAUUCCUUUUUCUCUCCUCUC | |
| | CCCAUGAUGUUUAGAAACACAACUAUAUUGUU | |
| | UGCUAAGCAUUCCAACUAUCUCAUUUCCAAGC | |
| | AAGUAUUAGAAUACCACAGGAACCACAAGACU | |
| | GCACAUCAAAAUAUGCCCCAUUCAACAUCUAG | |
| | UGAGCAGUCAGGAAAGAGAACUUCCAGAUCCU | |
| | GGAAAUCAGGGUUAGUAUUGUCCAGGUCUACC | |
| | AAAAAUCUCAAUAUUUCAGAUAAUCACAAUAC | |
| | AUCCCUUACCUGGGAAAGGGCUGUUAUAAUCU | |
| | UUCACAGGGGACAGGAUGGUUCCCUUGAUGAA | |
| | GAAGUUGAUAUGCCUUUUCCCAACUCCAGAAA | |
| | GUGACAAGCUCACAGACCUUUGAACUAGAGUU | |
| | UAGCUGGAAAAGUAUGUUAGUGCAAAUUGUCA | |
| | CAGGACAGCCCUUCUUUCCACAGAAGCUCCAG | |
| | GUAGAGGGUGUGUAAGUAGAUAGGCCAUGGGC | |
| | ACUGUGGGUAGACACACAUGAAGUCCAAGCAU | |
| | UUAGAUGUAUAGGUUGAUGGUGGUAUGUUUU | |
| | CAGGCUAGAUGUAUGUACUUCAUGCUGUCUAC | |
| | ACUAAGAGAGAAUGAGAGACACACUGAAGAAG | |
| | CACCAAUCAUGAAUUAGUUUUAUAUGCUUCUG | |
| | UUUUAUAAUUUUGUGAAGCAAAAUUUUUUCUC | |
| | UAGGAAAUAUUUAUUUUAAUAAUGUUUCAAAC | |
| | AUAUAUAACAAUGCUGUAUUUUAAAAGAAUGA | |
| | UUAUGAAUUACAUUUGUAUAAAAUAAUUUUU | |
| | AUAUUUGAAAUAUUGACUUUUUAUGGCACUAG | |
| | UAUUUCUAUGAAAUAUUAUGUUAAAACUGGGA | |
| | CAGGGGAGAACCUAGGGUGAUAUUAACCAGGG | |
| | GCCAUGAAUCACCUUUUGGUCUGGAGGGAAGC | |
| | CUUGGGGCUGAUGCAGUUGUUGCCCACAGCUG | |
| | UAUGAUUCCCAGCCAGCACAGCCUCUUAGAUG | |
| | CAGUUCUGAAGAAGAUGGUACCACCAGUCUGA | |
| | CUGUUUCCAUCAAGGGUACACUGCCUUCUCAA | |
| | CUCCAAACUGACUCUUAAGAAGACUGCAUUAU | |
| | AUUUAUUACUGUAAGAAAAUAUCACUUGUCAA | |
| | UAAAAUCCAUACAUUUGUGUGAA | |
| RESPIRATORY | UGAAUAAAAAUCUUAUAUUAAAAAUUCCCAUA | SEQ ID NO: 133 |
| SYNCYTIAL | GCUACACACUAACACUGUAUUCAAUUAUAGUU | |
| VIRUS (RSV) | AUUUAAAAUUAAAAAUUAUAUAAUUUUUUAA | |
| | UAACUUUUAGUGAACUAAUCCUAAAAUUAUCA | |
| | UUUUGAUCUAGGAGGAAUAAAUUUAAAUCCAA | |
| | AUCUAAUUGGUUUAUAUGUAUAUUAACUAAAC | |
| | UACGAGAUAUUAGUUUUUGACACUUUUUUUCU | |
| | CGU | |
| HUMAN | GAGGCAUUUAGGCCAUGGGACAGGGAGGACGC | SEQ ID NO: 134 |
| SURFACTANT- | UCUCUGGCCUUCGGCCUCCAUCCUGAGGCUCC | |
| A1 | ACUUGGUCUGUGAGAUGCUAGAACUCCCUUUC | |
| | AACAGAAUUCACUUGUGGCUAUUGGGACUGGA | |
| | GGCACCCUUAGCCACUUCAUUCCUCUGAUGGG | |
| | CCCUGACUCUUCCCCAUAAUCACUGACCAGCCU | |
| | UGACACUCCCCUUGCAAACUCUCCCAGCACUGC | |
| | ACCCCAGGCAGCCACUCUUAGCCUUGGCCUUC | |
| | GACAUGAGAUGGAGCCCUCCUUAUUCCCCAUC | |
| | UGGUCCAGUUCCUUCACUUACAGAUGGCAGCA | |
| | GUGAGGUCUUGGGGUAGAAGGACCCUCCAAAG | |
| | UCACACAAAGUGCCUGCCUCCUGGUCCCCUCA | |
| | GCUCUCUCUCUGCAACCCAGUGCCAUCAGGAU | |
| | GAGCAAUCCUGGCCAAGCAUAAUGACAGAGAG | |
| | AGGCAGACUUCGGGGAAGCCCUGACUGUGCAG | |
| | AGCUAAGGACACAGUGGAGAUUCUCUGGCACU | |
| | CUGAGGUCUCUGUGGCAGGCCUGGUCAGGCUC | |
| | UCCAUGAGGUUAGAAGGCCAGGUAGUGUUCCA | |
| | GCAGGGUGGUGGCCAAGCCAACCCCAUGAUUG | |
| | AUGUGUACGAUUCACUCCUUUGAGUCUUUGAA | |
| | UGGCAACUCAGCCCCCUGACCUGAAGACAGCC | |
| | AGCCUAGGCCUCUAGGGUGACCUAGAGCCGCC | |
| | UUCAGAUGUGACCCGAGUAACUUUCAACUGAU | |
| | GAACAAAUCUGCACCCUACUUCAGAUUUCAGU | |
| | GGGCAUUCACACCACCCCCCACACCACUGGCUC | |
| | UGCUUUCUCCUUUCAUUAAUCCAUUCACCCAG | |
| | AUAUUUCAUUAAAAUUAUCACGUGCCAGGUCU | |
| | UAGGAUAUGUCGUGGGGUGGGCAAGGUAAUCA | |
| | GUGACAGUUGAAGAUUUUUUUUUCCCAGAGCU | |
| | UAUGUCUUCAUCUGUGAAAUGGGAAUAAGAUA | |
| | CUUGUUGCUGUCACAGUUAUUACCAUCCCCCC | |
| | AGCUACCAAAAUUACUACCAGAACUGUUACUA | |
| | UACACAGAGGCUAUUGACUGAGCACCUAUCAU | |
| | UUGCCAAGAACCUUGACAAGCACUUCUAAUAC | |
| | AGCAUAUUAUGUACUAUUCAAUCUUUACACAA | |
| | UGUCACGGGACCAGUAUUGUUUCCUCAUUUUU | |
| | UAUAAGGACACUGAAGCUUGGAGGAGUUAAAU | |
| | GUUUUGAGUAUUAUUCCAGAGAGCAAGUGGCA | |
| | GAGGCUGGAUCCAAACCCAUCUUCCUGGACCU | |
| | GAAGCUUAUGCUUCCAGCCACCCCACUCCUGA | |
| | GCUGAAUAAAGAUGAUUUAAGCUUAAUAAAUC | |
| | GUGAAUGUGUUCACA | |
| HUMAN | GAGGCAUUUAGGCCAUGGGACAGGGAGGAUCC | SEQ ID NO: 135 |
| SURFACTANT | UGUCUGGCCUUCAGUUUCCAUCCCCAGGAUCC | |
| PROTEIN A2 | ACUUGGUCUGUGAGAUGCUAGAACUCCCUUUC | |
| | AACAGAAUUCACUUGUGGCUAUUAGAGCUGGA | |
| | GGCACCCUUAGCCACUUCAUUCCCCUGAUGGG | |
| | CCCUGACUCUUCCCCAUAAUCACUGACCAGCCU | |
| | UGACACUCCCCUUGCAAACCAUCCCAGCACUGC | |
| | ACCCCAGGCAGCCACUCCUAGCCUUGGCCUUU | |
| | GGCAUGAGAUGGAGGCCUCCUUAUUCCCCAUC | |
| | UGGUCCAGUUCCUUCACUUACAGAUGGCAGCA | |
| | GUGAGGCCUUGGGGUAGAAGGAUCCUCCAAAG | |
| | UCACACAGAGUGCCUGCCUCCUGGUCCCCUCA | |
| | GCUCUGCCUCUGCAGCCCACUGCCUGCCCAGUG | |
| | CCAUCAGGAUGAGCAGUACCGGCCAAGCAUAA | |
| | UGACAGAGAGAGGCAGAUUUCAGGGAAGCCCU | |
| | GACUGUGUGGAGCUAAGGACACAGUGGAGAUU | |
| | CUCUGGCACUCUGAGGUCUCUGUGGCAGGCCU | |
| | GGUCAGGCUCUCCAGGUGGUCAGAGGGCCCAG | |
| | UGGUGCCCCAGCACGGUGGUGCCCAAGCCAAC | |
| | CCUGUGACUGACAUGUACGAUUCACUCCUUUG | |
| | AGUCUUUGGAUGCCAACUCAGCCCCCUGACCU | |
| | GGAGGCAGCCGGCCAAGGCCUCUAGGGAAGAG | |
| | CCCCCCACUGCAGACAUGACCCGAGUAACUUU | |
| | CUGCUGAUGAACAAAUCUGCACCCCACUUCAG | |
| | ACCUCGGUGGGCAUUCACACCACCCCCCAUGCC | |
| | ACCGGCUCCACUUUCCCCUUUUAUUAAUACAU | |
| | UCACCCAGAUAAUCAUUAAAAUUAACAUGUGC | |
| | CAGGUCUUAGGAUGUGUCUUGGGGUGGGCACA | |
| | GUACCCGGUGACUCUUGGGGAUAUUUAUUUAU | |
| | UUUCCCUGAGCCUAUAUCUUCAUCUGUGAAAU | |
| | GGGGAUAAAAAUACUUGUUGCUGUCACAAUUA | |
| | UUACCAUCUCUCCAGCUAGCAAAAUUACUACC | |
| | AGAGCCGUUACUACACACAAAGGCUAUUGACC | |
| | GAGCACAUACCAUGUGCCACACACCUUGACAA | |
| | AAUCUUUUAAUACAGUUUAUUAUGUACUAUUC | |
| | AAUCUUUACACAAUGUCACGGGACCAGUAUUG | |
| | UUUACCCAAUUUUUUAUAAGGACACUGAAGCU | |
| | UAGAGGAGUGAAAUGUUUUGAGUGUUAUUUC | |
| | AGAGAGCAAAUGGCAAAGACUGGAUCCAAACC | |
| | CAUCUUCCUGGACCUGAAGUUCAUGCUCCCAG | |
| | CCACCCCACCCCUGAGCUGAAUAAAGAUGAUU | |
| | UAAGCAUAAUAAAUCGUUAGUGUGUUCACAUG | |
| | AGUUUCCAUA | |
| HUMAN | CGCCCAAGUGAAGCGCAUGCGCAGCGGGUGGU | SEQ ID NO: 136 |
| NAPSINA | CGCGGAGGUCCUGCUACCCAGUAAAAAUCCAC | |
| ASPARTIC | UAUUUCCAUUGA | |
| PEPTIDASE | | |
| (NAPSA) | | |
| HUMAN | AUGAAGAUCCAGCCGGCCUUGGGAGCCUGGAG | SEQ ID NO: 137 |
| CARBOXYLEST | GAGCAAAGACUGGGGUCUUUUGCGAAAGGGAU | |
| ERASE 1 (CES1) | UGCAGGUUCAGAAGGCAUCUUACCAUGGCUGG | |
| | GGAAUUGUCUGGUGGUGGGGGGCAGGGGACAG | |
| | AGGCCAUGAAGGAGCAAGUUUUGUAUUUGUGA | |
| | CCUCAGCUUUGGGAAUAAAGGAUCUUUUGAAG | |
| | GCCAA | |
| HUMAN | CAGCCCUGGUGUAUUUUCGAUAUUUCAGGAAG | SEQ ID NO: 138 |
| CEACAM6 | ACUGGCAGAUUGGACCAGACCCUGAAUUCUUC | |
| | UAGCUCCUCCAAUCCCAUUUUAUCCCAUGGAA | |
| | CCACUAAAAACAAGGUCUGCUCUGCUCCUGAA | |
| | GCCCUAUAUGCUGGAGAUGGACAACUCAAUGA | |
| | AAAUUUAAAGGGAAAACCCUCAGGCCUGAGGU | |
| | GUGUGCCACUCAGAGACUUCACCUAACUAGAG | |
| | ACAGGCAAACUGCAAACCAUGGUGAGAAAUUG | |
| | ACGACUUCACACUAUGGACAGCUUUUCCCAAG | |
| | AUGUCAAAACAAGACUCCUCAUCAUGAUAAGG | |
| | CUCUUACCCCCUUUUAAUUUGUCCUUGCUUAU | |
| | GCCUGCCUCUUUCGCUUGGCAGGAUGAUGCUG | |
| | UCAUUAGUAUUUCACAAGAAGUAGCUUCAGAG | |
| | GGUAACUUAACAGAGUAUCAGAUCUAUCUUGU | |
| | CAAUCCCAACGUUUUACAUAAAAUAAGAGAUC | |
| | CUUUAGUGCACCCAGUGACUGACAUUAGCAGC | |
| | AUCUUUAACACAGCCGUGUGUUCAAAUGUACA | |
| | GUGGUCCUUUUCAGAGUUGGACUUCUAGACUC | |
| | ACCUGUUCUCACUCCCUGUUUUAAUUCAACCC | |
| | AGCCAUGCAAUGCCAAAUAAUAGAAUUGCUCC | |
| | CUACCAGCUGAACAGGGAGGAGUCUGUGCAGU | |
| | UUCUGACACUUGUUGUUGAACAUGGCUAAAUA | |
| | CAAUGGGUAUCGCUGAGACUAAGUUGUAGAAA | |
| | UUAACAAAUGUGCUGCUUGGUUAAAAUGGCUA | |
| | CACUCAUCUGACUCAUUCUUUAUUCUAUUUUA | |
| | GUUGGUUUGUAUCUUGCCUAAGGUGCGUAGUC | |
| | CAACUCUUGGUAUUACCCUCCUAAUAGUCAUA | |
| | CUAGUAGUCAUACUCCCUGGUGUAGUGUAUUC | |
| | UCUAAAAGCUUUAAAUGUCUGCAUGCAGCCAG | |
| | CCAUCAAAUAGUGAAUGGUCUCUCUUUGGCUG | |
| | GAAUUACAAAACUCAGAGAAAUGUGUCAUCAG | |
| | GAGAACAUCAUAACCCAUGAAGGAUAAAAGCC | |
| | CCAAAUGGUGGUAACUGAUAAUAGCACUAAUG | |
| | CUUUAAGAUUUGGUCACACUCUCACCUAGGUG | |
| | AGCGCAUUGAGCCAGUGGUGCUAAAUGCUACA | |
| | UACUCCAACUGAAAUGUUAAGGAAGAAGAUAG | |
| | AUCCAAUUAAAAAAAAUUAAAACCAAUUUAAA | |
| | AAAAAAAAGAACACAGGAGAUUCCAGUCUACU | |
| | UGAGUUAGCAUAAUACAGAAGUCCCCUCUACU | |
| | UUAACUUUUACAAAAAAGUAACCUGAACUAAU | |
| | CUGAUGUUAACCAAUGUAUUUAUUUCUGUGGU | |
| | UCUGUUUCCUUGUUCCAAUUUGACAAAACCCA | |
| | CUGUUCUUGUAUUGUAUUGCCCAGGGGGAGCU | |
| | AUCACUGUACUUGUAGAGUGGUGCUGCUUUAA | |
| | UUCAUAAAUCACAAAUAAAAGCCAAUUAGCUC | |
| | UAUAACU | |
| HUMAN | CUGCCCGGGUGGCAUCCCUGUGACCCCUCCCCA | SEQ ID NO: 139 |
| GROWTH | GUGCCUCUCCUGGCCCUGGAAGUUGCCACUCC | |
| HORMONE | AGUGCCCACCAGCCUUGUCCUAAUAAAAUUAA | |
| | GUUGCAUCA | |
| INFLUENZA | GUGCAUUAAUUAAAAACACCCUUGUUUCUACU | SEQ ID NO: 140 |
| A/HA | | |
| INFLUENZA | AGAGAUAAGAUGGCUGAUUGAAGAAGUGAGAC | SEQ ID NO: 141 |
| A/NS1 | ACAGACUAAAAACAACUGAAAAUAGCUUUGAA | |
| | CAAAUAACAUUCAUGCAAGCAUUACAACUGCU | |
| | GUUUGAAGUGGAACAGGAGAUAAGAACUUUCU | |
| | CAUUUCAGCUUAUUUAAUGAUAAAAAACACCC | |
| | UUGUUUCUACU | |
| INFLUENZA | CCCGCUUGUUGUUGCCGCGAGUAUCAUUGGGA | SEQ ID NO: 142 |
| A/M1 | UCUUGCACUUGAUAUUGUGGAUUCUUGAUCGU | |
| | CUUUUUUUCAAAUGCGUCUAUCGACUCUUCAA | |
| | ACACGGCCUUAAAAGAGGCCCUUCUACGGAAG | |
| | GAGUACCUGAGUCUAUGAGGGAAGAAUAUCGA | |
| | AAGGAACAGCAGAAUGCUGUGGAUGCUGACGA | |
| | CAGUCAUUUUGUCAGCAUAGAGUUGGAGUAAA | |
| | AAACUACCUUGUUUCUACU | |
| INFLUENZA | GGAAAAAAUACCCUUGUUUCUACU | SEQ ID NO: 143 |
| A/NP | | |
| HUMAN HSP27 | AGCCUUAGCCCGGAUGCCCACCCCUGCUGCCGC | SEQ ID NO: 144 |
| (HSPB1) | CACUGGCUGUGCCUCCCCCGCCACCUGUGUGU | |
| | UCUUUUGAUACAUUUAUCUUCUGUUUUUCUCA | |
| | AAUAAAGUUCAAAGCAACCACCUGUCA | |
| HUMAN HSP70 | GCCAACCAAGUGUAGAUGUAGCAUUGUUCCAC | SEQ ID NO: 145 |
| | ACAUUUAAAACAUUUGAAGGACCUAAAUUCGU | |
| | AGCAAAUUCUGUGGCAGUUUUAAAAAGUUAAG | |
| | CUGCUAUAGUAAGUUACUGGGCAUUCUCAAUA | |
| | CUUGAAUAUGGAACAUAUGCACAGGGGAAGGA | |
| | AAUAACAUUGCACUUUAUAAACACUGUAUUGU | |
| | AAGUGGAAAAUGCAAUGUCUUAAAUAAAACUA | |
| | UUUAAAAUUGGCACCAUA | |

(Comparator Sequence
from U.S. Pat No. 9,181,321)
SEQ ID NO:146
AUGCAGCGGUCCCCGCUCGAAAAGGCCAGUGUCGUGUCCAA
ACUCUUCUUCUCAUGGACUCGGCCUAUCCUUAGAAAGGGG
UAUCGGCAGAGGCUUGAGUUGUCUGACAUCUACCAGAUCC
CCUCGGUAGAUUCGGCGGAUAACCUCUCGGAGAAGCUCGA
ACGGGAAUGGGACCGCGAACUCGCGUCUAAGAAAAACCCG
AAGCUCAUCAACGCACUGAGAAGGUGCUUCUUCUGGCGGU
UCAUGUUCUACGGUAUCUUCUUGUAUCUCGGGGAGGUCAC
AAAAGCAGUCCAACCCCUGUUGUUGGGUCGCAUUAUCGCC
UCGUACGACCCCGAUAACAAAGAAGAACGGAGCAUCGCGA
UCUACCUCGGGAUCGGACUGUGUUUGCUUUUCAUCGUCAG
AACACUUUUGUUGCAUCCAGCAAUCUUCGGCCUCCAUCAC
AUCGGUAUGCAGAUGCGAAUCGCUAUGUUUAGCUUGAUCU
ACAAAAAGACACUGAAACUCUCGUCGCGGGUGUUGGAUAA
GAUUUCCAUCGGUCAGUUGGUGUCCCUGCUUAGUAAUAAC
CUCAACAAAUUCGAUGAGGGACUGGCGCUGGCACAUUUCG
UGUGGAUUGCCCCGUUGCAAGUCGCCCUUUUGAUGGGCCU
UAUUUGGGAGCUGUUGCAGGCAUCUGCCUUUUGUGGCCUG
GGAUUUCUGAUUGUGUUGGCAUUGUUUCAGGCUGGGCUUG
GGCGGAUGAUGAUGAAGUAUCGCGACCAGAGAGCGGGUAA
AAUCUCGGAAAGACUCGUCAUCACUUCGGAAAUGAUCGAA
AACAUCCAGUCGGUCAAAGCCUAUUGCUGGGAAGAAGCUA
UGGAGAAGAUGAUUGAAAACCUCCGCCAAACUGAGCUGAA
ACUGACCCGCAAGGCGGCGUAUGUCCGGUAUUUCAAUUCG
UCAGCGUUCUUCUUUUCCGGGUUCUUCGUUGUCUUUCUCU
CGGUUUUGCCUUAUGCCUUGAUUAAGGGGAUUAUCCUCCG
CAAGAUUUUCACCACGAUUUCGUUCUGCAUUGUAUUGCGC
AUGGCAGUGACACGGCAAUUUCCGUGGGCCGUGCAGACAU
GGUAUGACUCGCUUGGAGCGAUCAACAAAAUCCAAGACUU
CUUGCAAAAGCAAGAGUACAAGACCCUGGAGUACAAUCUU
ACUACUACGGAGGUAGUAAUGGAGAAUGUGACGGCUUUUU
GGGAAGAGGGUUUUGGAGAACUGUUUGAGAAAGCAAAGCA
GAAUAACAACAACCGCAAGACCUCAAAUGGGGACGAUUCC
CUGUUUUUCUCGAACUUCUCCCUGCUCGGAACACCCGUGU
UGAAGGACAUCAAUUUCAAGAUUGAGAGGGGACAGCUUCU
CGCGGUAGCGGGAAGCACUGGUGCGGGAAAAACUAGCCUC
UUGAUGGUGAUUAUGGGGGAGCUUGAGCCCAGCGAGGGGA
AGAUUAAACACUCCGGGCGUAUCUCAUUCUGUAGCCAGUU
UUCAUGGAUCAUGCCCGGAACCAUUAAAGAGAACAUCAUU
UUCGGAGUAUCCUAUGAUGAGUACCGAUACAGAUCGGUCA
UUAAGGCGUGCCAGUUGGAAGAGGACAUUUCUAAGUUCGC
CGAGAAGGAUAACAUCGUCUUGGGAGAAGGGGGUAUUACA
UUGUCGGGAGGGCAGCGAGCGCGGAUCAGCCUCGCGAGAG
CGGUAUACAAAGAUGCAGAUUUGUAUCUGCUUGAUUCACC
GUUUGGAUACCUCGACGUAUUGACAGAAAAAGAAAUCUUC
GAGUCGUGCGUGUGUAAACUUAUGGCUAAUAAGACGAGAA
UCCUGGUGACAUCAAAAAUGGAACACCUUAAGAAGGCGGA
CAAGAUCCUGAUCCUCCACGAAGGAUCGUCCUACUUUUAC
GGCACUUUCUCAGAGUUGCAAAACUUGCAGCCGGACUUCU
CAAGCAAACUCAUGGGGUGUGACUCAUUCGACCAGUUCAG
CGCGGAACGGCGGAACUCGAUCUUGACGGAAACGCUGCAC
CGAUUCUCGCUUGAGGGUGAUGCCCCGGUAUCGUGGACCG
AGACAAAGAAGCAGUCGUUUAAGCAGACAGGAGAAUUUGG
UGAGAAAAGAAAGAACAGUAUCUUGAAUCCUAUUAACUCA
AUUCGCAAGUUCUCAAUCGUCCAGAAAACUCCACUGCAGA
UGAAUGGAAUUGAAGAGGAUUCGGACGAACCCCUGGAGCG
CAGGCUUAGCCUCGUGCCGGAUUCAGAGCAAGGGGAGGCC
AUUCUUCCCCGGAUUUCGGUGAUUUCAACCGGACCUACAC
UUCAGGCGAGGCGAAGGCAAUCCGUGCUCAACCUCAUGAC
GCAUUCGGUAAACCAGGGGCAAAACAUUCACCGCAAAACG
ACGGCCUCAACGAGAAAAGUGUCACUUGCACCCCAGGCGA
AUUUGACUGAACUCGACAUCUACAGCCGUAGGCUUUCGCA
AGAAACCGGACUUGAGAUCAGCGAAGAAAUCAAUGAAGAA
GAUUUGAAAGAGUGUUUCUUUGAUGACAUGGAAUCAAUCC
CAGCGGUGACAACGUGGAACACAUACUUGCGUUACAUCAC
GGUGCACAAGUCCUUGAUUUUCGUCCUCAUCUGGUGUCUC
GUGAUCUUUCUCGCUGAGGUCGCAGCGUCACUUGUGGUCC
UCUGGCUGCUUGGUAAUACGCCCUUGCAAGACAAAGGCAA
UUCUACACACUCAAGAAACAAUUCCUAUGCCGUGAUUAUC
ACUUCUACAAGCUCGUAUUACGUGUUUUACAUCUACGUAG
GAGUGGCCGACACUCUGCUCGCGAUGGGUUUCUUCCGAGG
ACUCCCACUCGUUCACACGCUUAUCACUGUCUCCAAGAUU
CUCCACCAUAAGAUGCUUCAUAGCGUACUGCAGGCUCCCA
UGUCCACCUUGAAUACGCUCAAGGCGGGAGGUAUUUUGAA
UCGCUUCUCAAAAGAUAUUGCAAUUUUGGAUGACCUUCUG
CCCCUGACGAUCUUCGACUUCAUCCAGUUGUUGCUGAUCG
UGAUUGGGGCUAUUGCAGUAGUCGCUGUCCUCCAGCCUUA
CAUUUUUGUCGCGACCGUUCCGGUGAUCGUGGCGUUUAUC
AUGCUGCGGGCCUAUUUCUUGCAGACGUCACAGCAGCUUA
AGCAACUGGAGUCUGAAGGGAGGUCGCCUAUCUUUACGCA
UCUUGUGACCAGUUUGAAGGGAUUGUGGACGUUGCGCGCC
UUUGGCAGGCAGCCCUACUUUGAAACACUGUUCCACAAAG
CGCUGAAUCUCCAUACGGCAAAUUGGUUUUUGUAUUUGAG
UACCCUCCGAUGGUUUCAGAUGCGCAUUGAGAUGAUUUUU
GUGAUCUUCUUUAUCGCGGUGACUUUUAUCUCCAUCUUGA
CCACGGGAGAGGGCGAGGGACGGGUCGGUAUUAUCCUGAC
ACUCGCCAUGAACAUUAUGAGCACUUUGCAGUGGGCAGUG
AACAGCUCGAUUGAUGUGGAUAGCCUGAUGAGGUCCGUUU
CGAGGGUCUUUAAGUUCAUCGACAUGCCGACGGAGGGAAA
GCCCACAAAAAGUACGAAACCCUAUAAGAAUGGGCAAUUG
AGUAAGGUAAUGAUCAUCGAGAACAGUCACGUGAAGAAGG
AUGACAUCUGGCCUAGCGGGGGUCAGAUGACCGUGAAGGA
CCUGACGGCAAAAUACACCGAGGGAGGGAACGCAAUCCUU
GAAAACAUCUCGUUCAGCAUUAGCCCCGGUCAGCGUGUGG
GGUUGCUCGGGAGGACCGGGUCAGGAAAAUCGACGUUGCU
GUCGGCCUUCUUGAGACUUCUGAAUACAGAGGGUGAGAUC
CAGAUCGACGGCGUUUCGUGGGAUAGCAUCACCUUGCAGC
AGUGGCGGAAAGCGUUUGGAGUAAUCCCCCAAAAGGUCUU
UAUCUUUAGCGGAACCUUCCGAAAGAAUCUCGAUCCUUAU
GAACAGUGGUCAGAUCAAGAGAUUUGGAAAGUCGCGGACG
AGGUUGGCCUUCGGAGUGUAAUCGAGCAGUUUCCGGGAAA
ACUCGACUUUGUCCUUGUAGAUGGGGGAUGCGUCCUGUCG
CAUGGGCACAAGCAGCUCAUGUGCCUGGCGCGAUCCGUCC
UCUCUAAAGCGAAAAUUCUUCUCUUGGAUGAACCUUCGGC
CCAUCUGGACCCGGUAACGUAUCAGAUCAUCAGAAGGACA
CUUAAGCAGGCGUUUGCCGACUGCACGGUGAUUCUCUGUG
AGCAUCGUAUCGAGGCCAUGCUCGAAUGCCAGCAAUUUCU
UGUCAUCGAAGAGAAUAAGGUCCGCCAGUACGACUCCAUC
CAGAAGCUGCUUAAUGAGAGAUCAUUGUUCCGGCAGGCGA
UUUCACCAUCCGAUAGGGUGAAACUUUUUCCACACAGAAA
UUCGUCGAAGUGCAAGUCCAAACCGCAGAUCGCGGCCUUG
AAAGAAGAGACUGAAGAAGAAGUUCAAGACACGCGUCUUU
AA

1. A composition comprising
a. a lipid formulation comprising
i. about 20 mol % to about 30 mol % of an ionizable cationic lipid having the structure of ATX-012: 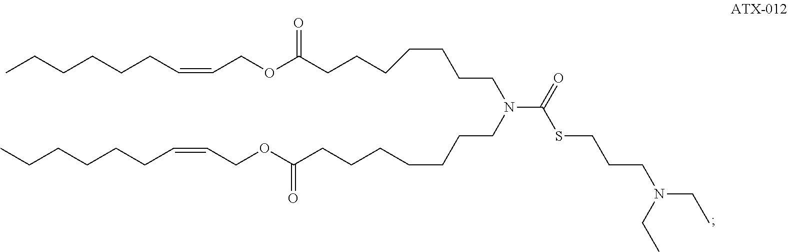 [SMILES: CCCCCC/C=C\COC(=O)CCCCCCCN(CCCCCCCC(=O)OC/C=C\CCCCCC)C(=O)SCCCN(CC)CC]
ii. about 20 mol % to about 30 mol % 1,2-Dioleoyl-3-Trimethylammonium-Propane (DOTAP);
iii. about 7 mol % to about 13 mol % of a helper lipid;
iv. about 33 mol % to about 44 mol % cholesterol; and
v. about 0.5 mol % to about 3.0 mol % of a PEG-lipid conjugate; and
b. a messenger RNA (mRNA) encoding a peptide having cystic fibrosis transmembrane conductance regulator (CFTR) activity;
wherein the lipid formulation encapsulates the mRNA.

2. The composition of claim 1, wherein the lipid formulation is selected from the group consisting of a lipoplex, a liposome, a lipid nanoparticle, a polymer-based carrier, an exosome, a lamellar body, a micelle and an emulsion.

3. The composition of claim 1, wherein the lipid formulation is a liposome selected from the group consisting of a cationic liposome, a nanoliposome, a proteoliposome, a unilamellar liposome, a multilamellar liposome, a ceramide-containing nanoliposome and a multivesicular liposome.

4. The composition of claim 2, wherein the lipid formulation is a lipid nanoparticle.

5. The composition of claim 4, wherein the lipid nanoparticle has a size of less than about 200 nm.

6. (canceled)

7. (canceled)

8. The composition of claim 4, wherein the lipid nanoparticle has a size of about 55 nm to about 90 nm.

9. The composition of claim 1, wherein the helper lipid is a phospholipid.

10. The composition of claim 9, wherein the helper lipid is selected from the group consisting of dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC) and phosphatidylcholine (PC).

11. (canceled)

12. The composition of claim 1, wherein the PEG-lipid conjugate is PEG-DMG.

13. The composition of claim 12, wherein the PEG-DMG is PEG2000-DMG.

14. The composition of claim 1, wherein the composition has a total lipid:mRNA weight ratio of about 5:1 to about 25:1.

15. (canceled)

16. (canceled)

17. (canceled)

18. The composition of claim 1, wherein the lipid formulation comprises about 22 mol % to about 28 mol % of the ionizable cationic lipid.

19. (canceled)

20. (canceled)

21. The composition of claim 1, wherein the lipid formulation comprises about 22 mol % to about 28 mol % DOTAP.

22. (canceled)

23. (canceled)

24. The composition of claim 1, wherein the lipid formulation comprises about 8 mol % to about 12 mol % of the helper lipid.

25. (canceled)

26. The composition of claim 1, wherein the lipid formulation comprises about 35 mol % to about 41 mol % cholesterol.

27. (canceled)

28. The composition of claim 1, wherein the lipid formulation comprises about 0.75 mol % to about 2.5 mol % of the PEG-lipid conjugate.

29. (canceled)

30. (canceled)

31. The composition of claim 1, wherein the peptide having CFTR activity has a sequence at least about 85% identical to a sequence of SEQ ID NO: 99.

32. (canceled)

33. (canceled)

34. (canceled)

35. (canceled)

36. The composition of claim 31, wherein the peptide having CFTR activity has a sequence of SEQ ID NO: 99.

37. The composition of claim 1, wherein the mRNA has a sequence selected from the group consisting of SEQ ID NOs: 49, 53, 66, 68, 69 and 72.

38. (canceled)

39. (canceled)

40. (canceled)

41. (canceled)

42. (canceled)

43. (canceled)

44. The composition of claim 1, wherein the mRNA comprises a 3′ poly-A tail consisting of about 50 to about 120 adenosine monomers.

45. The composition of claim 1, wherein the mRNA comprises a 5′ cap.

46. The composition of claim 45, wherein the 5′ cap is m⁷GpppAmpG having the structure of Formula (Cap V): 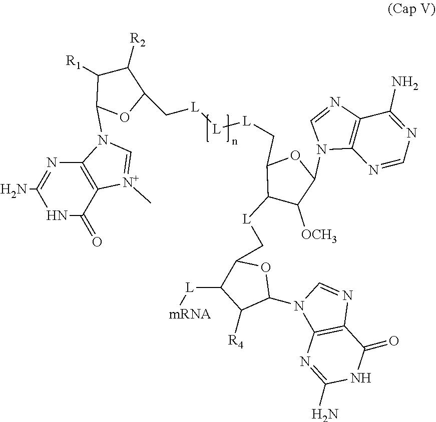
wherein R¹, R², and R⁴ are each OH, n is 1, each L is a phosphate linked by diester bonds, and mRNA is the mRNA of the composition.

47. The composition of claim 1, wherein the mRNA comprises one or more chemically-modified nucleotides each independently selected from the group consisting of 5-hydroxycytidine, 5-methylcytidine, 5-hydroxymethylcytidine, 5-carboxycytidine, 5-formylcytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-hydroxymethyluridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N¹-hydroxypseudouridine, N¹-methylpseudouridine, 2′-O-methyl-N¹-methylpseudouridine, N¹-ethylpseudouridine, N¹-hydroxymethylpseudouridine, arauridine, N⁶-methyladenosine, 2-aminoadenosine, 3-methyladenosine, 7-deazaadenosine, 8-oxoadenosine, inosine, thienoguanosine, 7-deazaguanosine, 8-oxoguanosine and 6-O-methylguanosine.

48. (canceled)

49. The composition of claim 1, wherein the composition comprises a HEPES or TRIS buffer at a pH of about 7.0 to about 8.5.

50. (canceled)

51. The composition of claim 49, wherein the HEPES or TRIS buffer is at a concentration of about 20 mM to about 80 mM.

52. (canceled)

53. (canceled)

54. (canceled)

55. (canceled)

56. (canceled)

57. (canceled)

58. The composition of claim 49, wherein the composition further comprises about 10 mM to about 100 mM of NaCl.

59. (canceled)

60. (canceled)

61. (canceled)

62. (canceled)

63. (canceled)

64. The composition of claim 1, wherein the composition further comprises one or more cryoprotectants.

65. The composition of claim 64, wherein the one or more cryoprotectants are selected from the group consisting of sucrose, glycerol, and a combination of sucrose and glycerol.

66. The composition of claim 65, wherein the composition comprises a combination of sucrose at a concentration of about 5% w/v to about 18% w/v and glycerol at a concentration of about 1% w/v to about 9% w/v.

67. (canceled)

68. (canceled)

69. (canceled)

70. (canceled)

71. The composition of claim 1, wherein:
the helper lipid is distearoylphosphatidylcholine (DSPC);
the PEG-lipid conjugate is PEG2000-DMG; and
the mRNA comprises SEQ ID NO: 53.

72. The composition of claim 71, wherein the lipid formulation is a lipid nanoparticle.

73. The composition of claim 72, wherein the lipid nanoparticle has a size of less than about 100 nm.

74. (canceled)

75. (canceled)

76. (canceled)

77. (canceled)

78. (canceled)

79. (canceled)

80. (canceled)

81. (canceled)

82. A method for ameliorating, preventing, delaying onset, or treating a disease or disorder associated with reduced activity of Cystic Fibrosis Transmembrane Conductance Regulator (CFTR) in a subject in need thereof, the method comprising administering to the subject a composition of claim 1.

83. (canceled)

84. (canceled)

85. (canceled)

86. (canceled)

87. (canceled)

88. (canceled)

89. (canceled)

90. A method of expressing a CFTR protein in a cell comprising contacting the cell with a composition of claim 1.

91. A kit for expressing a human CFTR in vivo, the kit comprising a composition of claim 1 and a device for administering the dose.

92. (canceled)

93. (canceled)
